SEQUENCE LISTING (1251 sequences)

SEQ ID NO: 1 - RNA, 17 nt, synthetic construct
  source 1..17: mol_type = other RNA
ctgggttggggcaaaga

SEQ ID NO: 2 - RNA, 18 nt, synthetic construct
  source 1..18: mol_type = other RNA
cctgggttggggcaaaga

SEQ ID NO: 3 - RNA, 19 nt, synthetic construct
  source 1..19: mol_type = other RNA
gcctgggttggggcaaaga

SEQ ID NO: 4 - RNA, 20 nt, synthetic construct
  source 1..20: mol_type = other RNA
agcctgggttggggcaaaga

SEQ ID NO: 5 - RNA, 21 nt, synthetic construct
  source 1..21: mol_type = other RNA
cagcctgggttggggcaaaga

SEQ ID NO: 6 - RNA, 22 nt, synthetic construct
  source 1..22: mol_type = other RNA
ccagcctgggttggggcaaaga

SEQ ID NO: 7-11 - skipped (5 entries)

SEQ ID NO: 12 - RNA, 10 nt, synthetic construct
  source 1..10: mol_type = other RNA
ttgccccaac

SEQ ID NO: 13 - RNA, 11 nt, synthetic construct
  source 1..11: mol_type = other RNA
ttgccccaacc

SEQ ID NO: 14 - RNA, 12 nt, synthetic construct
  source 1..12: mol_type = other RNA
ttgccccaaccc

SEQ ID NO: 15 - RNA, 13 nt, synthetic construct
  source 1..13: mol_type = other RNA
ttgccccaaccca

SEQ ID NO: 16 - RNA, 14 nt, synthetic construct
  source 1..14: mol_type = other RNA
ttgccccaacccag

SEQ ID NO: 17 - RNA, 15 nt, synthetic construct
  source 1..15: mol_type = other RNA
ttgccccaacccagg

SEQ ID NO: 18 - RNA, 16 nt, synthetic construct
  source 1..16: mol_type = other RNA
ttgccccaacccaggc

SEQ ID NO: 19 - RNA, 17 nt, synthetic construct
  source 1..17: mol_type = other RNA
ttgccccaacccaggct

SEQ ID NO: 20 - RNA, 18 nt, synthetic construct
  source 1..18: mol_type = other RNA
ttgccccaacccaggctg

SEQ ID NO: 21 - RNA, 19 nt, synthetic construct
  source 1..19: mol_type = other RNA
ttgccccaacccaggctgg

SEQ ID NO: 22 - RNA, 27 nt, synthetic construct
  source 1..27: mol_type = other RNA
tacgcccttcgagcatgtaggtcacgg

SEQ ID NO: 23 - RNA, 29 nt, synthetic construct
  source 1..29: mol_type = other RNA
acgacggagaccgccgtcgtcgacaagcc

SEQ ID NO: 24 - RNA, 30 nt, synthetic construct
  source 1..30: mol_type = other RNA
gacgacggagaccgccgtcgtcgacaagcc

SEQ ID NO: 25 - RNA, 30 nt, synthetic construct
  source 1..30: mol_type = other RNA
gcatacgcccttcgagcatgtaggtcacgg

SEQ ID NO: 26 - RNA, 33 nt, synthetic construct
  source 1..33: mol_type = other RNA
ggcgcatacgcccttcgagcatgtaggtcacgg

SEQ ID NO: 27 - RNA, 38 nt, synthetic construct
  source 1..38: mol_type = other RNA
atgatcctgacgacggagaccgccgtcgtcgacaagcc

SEQ ID NO: 28 - RNA, 39 nt, synthetic construct
  source 1..39: mol_type = other RNA
cttcgtggcgcatacgcccttcgagcatgtaggtcacgg

SEQ ID NO: 29 - RNA, 132 nt, synthetic construct
  source 1..132: mol_type = other RNA
agcctgggttggggcaaagagttttagagctagaaatagcaagttaaaataaggctagtccgttatcaacttgaaaaagtggcaccgagtcggtgcacgacggagaccgccgtcgtcgacaagccttgcccc

SEQ ID NO: 30 - RNA, 136 nt, synthetic construct
  source 1..136: mol_type = other RNA
agcctgggttggggcaaagagttttagagctagaaatagcaagttaaaataaggctagtccgttatcaacttgaaaaagtggcaccgagtcggtgcacgacggagaccgccgtcgtcgacaagccttgccccaacc

SEQ ID NO: 31 - RNA, 136 nt, synthetic construct
  source 1..136: mol_type = other RNA
agcctgggttggggcaaagagttttagagctagaaatagcaagttaaaataaggctagtccgttatcaacttgaaaaagtggcaccgagtcggtgctacgcccttcgagcatgtaggtcacggttgccccaaccca

SEQ ID NO: 32 - RNA, 137 nt, synthetic construct
  source 1..137: mol_type = other RNA
agcctgggttggggcaaagagttttagagctagaaatagcaagttaaaataaggctagtccgttatcaacttgaaaaagtggcaccgagtcggtgcgacgacggagaccgccgtcgtcgacaagccttgccccaacc

SEQ ID NO: 33 - RNA, 139 nt, synthetic construct
  source 1..139: mol_type = other RNA
agcctgggttggggcaaagagttttagagctagaaatagcaagttaaaataaggctagtccgttatcaacttgaaaaagtggcaccgagtcggtgcgcatacgcccttcgagcatgtaggtcacggttgccccaaccca

SEQ ID NO: 34 - RNA, 139 nt, synthetic construct
  source 1..139: mol_type = other RNA
agcctgggttggggcaaagagttttagagctagaaatagcaagttaaaataaggctagtccgttatcaacttgaaaaagtggcaccgagtcggtgcgacgacggagaccgccgtcgtcgacaagccttgccccaaccca

SEQ ID NO: 35 - RNA, 140 nt, synthetic construct
  source 1..140: mol_type = other RNA
agcctgggttggggcaaagagttttagagctagaaatagcaagttaaaataaggctagtccgttatcaacttgaaaaagtggcaccgagtcggtgctacgcccttcgagcatgtaggtcacggttgccccaacccatttt

SEQ ID NO: 36 - RNA, 141 nt, synthetic construct
  source 1..141: mol_type = other RNA
agcctgggttggggcaaagagttttagagctagaaatagcaagttaaaataaggctagtccgttatcaacttgaaaaagtggcaccgagtcggtgcgacgacggagaccgccgtcgtcgacaagccttgccccaacctttt

SEQ ID NO: 37 - RNA, 141 nt, synthetic construct
  source 1..141: mol_type = other RNA
agcctgggttggggcaaagagttttagagctagaaatagcaagttaaaataaggctagtccgttatcaacttgaaaaagtggcaccgagtcggtgcatgatcctgacgacggagaccgccgtcgtcgacaagccttgcccc

SEQ ID NO: 38 - RNA, 141 nt, synthetic construct
  source 1..141: mol_type = other RNA
agcctgggttggggcaaagagttttagagctagaaatagcaagttaaaataaggctagtccgttatcaacttgaaaaagtggcaccgagtcggtgcgacgacggagaccgccgtcgtcgacaagccttgccccaacccagg

SEQ ID NO: 39 - RNA, 142 nt, synthetic construct
  source 1..142: mol_type = other RNA
agcctgggttggggcaaagagttttagagctagaaatagcaagttaaaataaggctagtccgttatcaacttgaaaaagtggcaccgagtcggtgcggcgcatacgcccttcgagcatgtaggtcacggttgccccaaccca

SEQ ID NO: 40 - RNA, 143 nt, synthetic construct
  source 1..143: mol_type = other RNA
agcctgggttggggcaaagagttttagagctagaaatagcaagttaaaataaggctagtccgttatcaacttgaaaaagtggcaccgagtcggtgcgcatacgcccttcgagcatgtaggtcacggttgccccaacccatttt

SEQ ID NO: 41 - RNA, 143 nt, synthetic construct
  source 1..143: mol_type = other RNA
agcctgggttggggcaaagagttttagagctagaaatagcaagttaaaataaggctagtccgttatcaacttgaaaaagtggcaccgagtcggtgcgacgacggagaccgccgtcgtcgacaagccttgccccaacccatttt

SEQ ID NO: 42 - RNA, 143 nt, synthetic construct
  source 1..143: mol_type = other RNA
agcctgggttggggcaaagagttttagagctagaaatagcaagttaaaataaggctagtccgttatcaacttgaaaaagtggcaccgagtcggtgcgacgacggagaccgccgtcgtcgacaagccttgccccaacccaggct

SEQ ID NO: 43 - RNA, 143 nt, synthetic construct
  source 1..143: mol_type = other RNA
agcctgggttggggcaaagagttttagagctagaaatagcaagttaaaataaggctagtccgttatcaacttgaaaaagtggcaccgagtcggtgcatgatcctgacgacggagaccgccgtcgtcgacaagccttgccccaa

SEQ ID NO: 44 - RNA, 145 nt, synthetic construct
  source 1..145: mol_type = other RNA
agcctgggttggggcaaagagttttagagctagaaatagcaagttaaaataaggctagtccgttatcaacttgaaaaagtggcaccgagtcggtgcatgatcctgacgacggagaccgccgtcgtcgacaagccttgccccaacc

SEQ ID NO: 45 - RNA, 145 nt, synthetic construct
  source 1..145: mol_type = other RNA
agcctgggttggggcaaagagttttagagctagaaatagcaagttaaaataaggctagtccgttatcaacttgaaaaagtggcaccgagtcggtgcgacgacggagaccgccgtcgtcgacaagccttgccccaacccaggtttt

SEQ ID NO: 46 - RNA, 146 nt, synthetic construct
  source 1..146: mol_type = other RNA
agcctgggttggggcaaagagttttagagctagaaatagcaagttaaaataaggctagtccgttatcaacttgaaaaagtggcaccgagtcggtgcggcgcatacgcccttcgagcatgtaggtcacggttgccccaacccatttt

SEQ ID NO: 47 - RNA, 147 nt, synthetic construct
  source 1..147: mol_type = other RNA
agcctgggttggggcaaagagttttagagctagaaatagcaagttaaaataaggctagtccgttatcaacttgaaaaagtggcaccgagtcggtgcgacgacggagaccgccgtcgtcgacaagccttgccccaacccaggcttttt

SEQ ID NO: 48 - RNA, 147 nt, synthetic construct
  source 1..147: mol_type = other RNA
agcctgggttggggcaaagagttttagagctagaaatagcaagttaaaataaggctagtccgttatcaacttgaaaaagtggcaccgagtcggtgcatgatcctgacgacggagaccgccgtcgtcgacaagccttgccccaatttt

SEQ ID NO: 49 - RNA, 147 nt, synthetic construct
  source 1..147: mol_type = other RNA
agcctgggttggggcaaagagttttagagctagaaatagcaagttaaaataaggctagtccgttatcaacttgaaaaagtggcaccgagtcggtgcatgatcctgacgacggagaccgccgtcgtcgacaagccttgccccaaccca

SEQ ID NO: 50 - RNA, 148 nt, synthetic construct
  source 1..148: mol_type = other RNA
agcctgggttggggcaaagagttttagagctagaaatagcaagttaaaataaggctagtccgttatcaacttgaaaaagtggcaccgagtcggtgccttcgtggcgcatacgcccttcgagcatgtaggtcacggttgccccaaccca

SEQ ID NO: 51 - RNA, 149 nt, synthetic construct
  source 1..149: mol_type = other RNA
agcctgggttggggcaaagagttttagagctagaaatagcaagttaaaataaggctagtccgttatcaacttgaaaaagtggcaccgagtcggtgcatgatcctgacgacggagaccgccgtcgtcgacaagccttgccccaacctttt

SEQ ID NO: 52 - RNA, 149 nt, synthetic construct
  source 1..149: mol_type = other RNA
agcctgggttggggcaaagagttttagagctagaaatagcaagttaaaataaggctagtccgttatcaacttgaaaaagtggcaccgagtcggtgcatgatcctgacgacggagaccgccgtcgtcgacaagccttgccccaacccagg

SEQ ID NO: 53 - RNA, 151 nt, synthetic construct
  source 1..151: mol_type = other RNA
agcctgggttggggcaaagagttttagagctagaaatagcaagttaaaataaggctagtccgttatcaacttgaaaaagtggcaccgagtcggtgcatgatcctgacgacggagaccgccgtcgtcgacaagccttgccccaacccatttt

SEQ ID NO: 54 - RNA, 151 nt, synthetic construct
  source 1..151: mol_type = other RNA
agcctgggttggggcaaagagttttagagctagaaatagcaagttaaaataaggctagtccgttatcaacttgaaaaagtggcaccgagtcggtgcatgatcctgacgacggagaccgccgtcgtcgacaagccttgccccaacccaggct

SEQ ID NO: 55 - RNA, 152 nt, synthetic construct
  source 1..152: mol_type = other RNA
agcctgggttggggcaaagagttttagagctagaaatagcaagttaaaataaggctagtccgttatcaacttgaaaaagtggcaccgagtcggtgccttcgtggcgcatacgcccttcgagcatgtaggtcacggttgccccaacccatttt

SEQ ID NO: 56 - RNA, 153 nt, synthetic construct
  source 1..153: mol_type = other RNA
agcctgggttggggcaaagagttttagagctagaaatagcaagttaaaataaggctagtccgttatcaacttgaaaaagtggcaccgagtcggtgcatgatcctgacgacggagaccgccgtcgtcgacaagccttgccccaacccaggtttt

SEQ ID NO: 57 - RNA, 155 nt, synthetic construct
  source 1..155: mol_type = other RNA
agcctgggttggggcaaagagttttagagctagaaatagcaagttaaaataaggctagtccgttatcaacttgaaaaagtggcaccgagtcggtgcatgatcctgacgacggagaccgccgtcgtcgacaagccttgccccaacccaggcttttt

SEQ ID NO: 58 - RNA, 17 nt, synthetic construct
  source 1..17: mol_type = other RNA
cctgggttggggcaaag

SEQ ID NO: 59 - RNA, 18 nt, synthetic construct
  source 1..18: mol_type = other RNA
gcctgggttggggcaaag

SEQ ID NO: 60 - RNA, 19 nt, synthetic construct
  source 1..19: mol_type = other RNA
agcctgggttggggcaaag

SEQ ID NO: 61 - RNA, 20 nt, synthetic construct
  source 1..20: mol_type = other RNA
cagcctgggttggggcaaag

SEQ ID NO: 62 - RNA, 21 nt, synthetic construct
  source 1..21: mol_type = other RNA
ccagcctgggttggggcaaag

SEQ ID NO: 63 - RNA, 22 nt, synthetic construct
  source 1..22: mol_type = other RNA
tccagcctgggttggggcaaag

SEQ ID NO: 64-68 - skipped (5 entries)

SEQ ID NO: 69 - RNA, 10 nt, synthetic construct
  source 1..10: mol_type = other RNA
tgccccaacc

SEQ ID NO: 70 - RNA, 11 nt, synthetic construct
  source 1..11: mol_type = other RNA
tgccccaaccc

SEQ ID NO: 71 - RNA, 12 nt, synthetic construct
  source 1..12: mol_type = other RNA
tgccccaaccca

SEQ ID NO: 72 - RNA, 13 nt, synthetic construct
  source 1..13: mol_type = other RNA
tgccccaacccag

SEQ ID NO: 73 - RNA, 14 nt, synthetic construct
  source 1..14: mol_type = other RNA
tgccccaacccagg

SEQ ID NO: 74 - RNA, 15 nt, synthetic construct
  source 1..15: mol_type = other RNA
tgccccaacccaggc

SEQ ID NO: 75 - RNA, 16 nt, synthetic construct
  source 1..16: mol_type = other RNA
tgccccaacccaggct

SEQ ID NO: 76 - RNA, 17 nt, synthetic construct
  source 1..17: mol_type = other RNA
tgccccaacccaggctg

SEQ ID NO: 77 - RNA, 18 nt, synthetic construct
  source 1..18: mol_type = other RNA
tgccccaacccaggctgg

SEQ ID NO: 78 - RNA, 19 nt, synthetic construct
  source 1..19: mol_type = other RNA
tgccccaacccaggctgga

SEQ ID NO: 79 - RNA, 142 nt, synthetic construct
  source 1..142: mol_type = other RNA
cagcctgggttggggcaaaggttttagagctagaaatagcaagttaaaataaggctagtccgttatcaacttgaaaaagtggcaccgagtcggtgcatgatcctgacgacggagaccgccgtcgtcgacaagcctgccccaa

SEQ ID NO: 80 - RNA, 144 nt, synthetic construct
  source 1..144: mol_type = other RNA
cagcctgggttggggcaaaggttttagagctagaaatagcaagttaaaataaggctagtccgttatcaacttgaaaaagtggcaccgagtcggtgcatgatcctgacgacggagaccgccgtcgtcgacaagcctgccccaacc

SEQ ID NO: 81 - RNA, 146 nt, synthetic construct
  source 1..146: mol_type = other RNA
cagcctgggttggggcaaaggttttagagctagaaatagcaagttaaaataaggctagtccgttatcaacttgaaaaagtggcaccgagtcggtgcatgatcctgacgacggagaccgccgtcgtcgacaagcctgccccaatttt

SEQ ID NO: 82 - RNA, 146 nt, synthetic construct
  source 1..146: mol_type = other RNA
cagcctgggttggggcaaaggttttagagctagaaatagcaagttaaaataaggctagtccgttatcaacttgaaaaagtggcaccgagtcggtgcatgatcctgacgacggagaccgccgtcgtcgacaagcctgccccaaccca

SEQ ID NO: 83 - RNA, 148 nt, synthetic construct
  source 1..148: mol_type = other RNA
cagcctgggttggggcaaaggttttagagctagaaatagcaagttaaaataaggctagtccgttatcaacttgaaaaagtggcaccgagtcggtgcatgatcctgacgacggagaccgccgtcgtcgacaagcctgccccaacctttt

SEQ ID NO: 84 - RNA, 150 nt, synthetic construct
  source 1..150: mol_type = other RNA
cagcctgggttggggcaaaggttttagagctagaaatagcaagttaaaataaggctagtccgttatcaacttgaaaaagtggcaccgagtcggtgcatgatcctgacgacggagaccgccgtcgtcgacaagcctgccccaacccatttt

SEQ ID NO: 85 - RNA, 17 nt, synthetic construct
  source 1..17: mol_type = other RNA
gactccagcctgggttg

SEQ ID NO: 86 - RNA, 18 nt, synthetic construct
  source 1..18: mol_type = other RNA
ggactccagcctgggttg

SEQ ID NO: 87 - RNA, 19 nt, synthetic construct
  source 1..19: mol_type = other RNA
tggactccagcctgggttg

SEQ ID NO: 88 - RNA, 20 nt, synthetic construct
  source 1..20: mol_type = other RNA
ctggactccagcctgggttg

SEQ ID NO: 89 - RNA, 21 nt, synthetic construct
  source 1..21: mol_type = other RNA
tctggactccagcctgggttg

SEQ ID NO: 90 - RNA, 22 nt, synthetic construct
  source 1..22: mol_type = other RNA
atctggactccagcctgggttg

SEQ ID NO: 91-95 - skipped (5 entries)

SEQ ID NO: 96 - RNA, 10 nt, synthetic construct
  source 1..10: mol_type = other RNA
cccaggctgg

SEQ ID NO: 97 - RNA, 11 nt, synthetic construct
  source 1..11: mol_type = other RNA
cccaggctgga

SEQ ID NO: 98 - RNA, 12 nt, synthetic construct
  source 1..12: mol_type = other RNA
cccaggctggag

SEQ ID NO: 99 - RNA, 13 nt, synthetic construct
  source 1..13: mol_type = other RNA
cccaggctggagt

SEQ ID NO: 100 - RNA, 14 nt, synthetic construct
  source 1..14: mol_type = other RNA
cccaggctggagtc

SEQ ID NO: 101 - RNA, 15 nt, synthetic construct
  source 1..15: mol_type = other RNA
cccaggctggagtcc

SEQ ID NO: 102 - RNA, 16 nt, synthetic construct
  source 1..16: mol_type = other RNA
cccaggctggagtcca

SEQ ID NO: 103 - RNA, 17 nt, synthetic construct
  source 1..17: mol_type = other RNA
cccaggctggagtccag

SEQ ID NO: 104 - RNA, 18 nt, synthetic construct
  source 1..18: mol_type = other RNA
cccaggctggagtccaga

SEQ ID NO: 105 - RNA, 19 nt, synthetic construct
  source 1..19: mol_type = other RNA
cccaggctggagtccagat

SEQ ID NO: 106 - RNA, 30 nt, synthetic construct
  source 1..30: mol_type = other RNA
gacgacggcggtctccgtcgtcaggatcat

SEQ ID NO: 107 - RNA, 38 nt, synthetic construct
  source 1..38: mol_type = other RNA
ggcttgtcgacgacggcggtctccgtcgtcaggatcat

SEQ ID NO: 108 - RNA, 131 nt, synthetic construct
  source 1..131: mol_type = other RNA
ctggactccagcctgggttggttttagagctagaaatagcaagttaaaataaggctagtccgttatcaacttgaaaaagtggcaccgagtcggtgcacgacggagaccgccgtcgtcgacaagcccccagg

SEQ ID NO: 109 - RNA, 135 nt, synthetic construct
  source 1..135: mol_type = other RNA
ctggactccagcctgggttggttttagagctagaaatagcaagttaaaataaggctagtccgttatcaacttgaaaaagtggcaccgagtcggtgcacgacggagaccgccgtcgtcgacaagcccccaggctgg

SEQ ID NO: 110 - RNA, 136 nt, synthetic construct
  source 1..136: mol_type = other RNA
ctggactccagcctgggttggttttagagctagaaatagcaagttaaaataaggctagtccgttatcaacttgaaaaagtggcaccgagtcggtgctacgcccttcgagcatgtaggtcacggcccaggctggagt

SEQ ID NO: 111 - RNA, 137 nt, synthetic construct
  source 1..137: mol_type = other RNA
ctggactccagcctgggttggttttagagctagaaatagcaagttaaaataaggctagtccgttatcaacttgaaaaagtggcaccgagtcggtgcgacgacggagaccgccgtcgtcgacaagcccccaggctgga

SEQ ID NO: 112 - RNA, 137 nt, synthetic construct
  source 1..137: mol_type = other RNA
ctggactccagcctgggttggttttagagctagaaatagcaagttaaaataaggctagtccgttatcaacttgaaaaagtggcaccgagtcggtgcgacgacggcggtctccgtcgtcaggatcatcccaggctgga

SEQ ID NO: 113 - RNA, 139 nt, synthetic construct
  source 1..139: mol_type = other RNA
ctggactccagcctgggttggttttagagctagaaatagcaagttaaaataaggctagtccgttatcaacttgaaaaagtggcaccgagtcggtgcgcatacgcccttcgagcatgtaggtcacggcccaggctggagt

SEQ ID NO: 114 - RNA, 139 nt, synthetic construct
  source 1..139: mol_type = other RNA
ctggactccagcctgggttggttttagagctagaaatagcaagttaaaataaggctagtccgttatcaacttgaaaaagtggcaccgagtcggtgcgacgacggagaccgccgtcgtcgacaagcccccaggctggagt

SEQ ID NO: 115 - RNA, 139 nt, synthetic construct
  source 1..139: mol_type = other RNA
ctggactccagcctgggttggttttagagctagaaatagcaagttaaaataaggctagtccgttatcaacttgaaaaagtggcaccgagtcggtgcgacgacggcggtctccgtcgtcaggatcatcccaggctggagt

SEQ ID NO: 116 - RNA, 140 nt, synthetic construct
  source 1..140: mol_type = other RNA
ctggactccagcctgggttggttttagagctagaaatagcaagttaaaataaggctagtccgttatcaacttgaaaaagtggcaccgagtcggtgcatgatcctgacgacggagaccgccgtcgtcgacaagcccccagg

SEQ ID NO: 117 - RNA, 141 nt, synthetic construct
  source 1..141: mol_type = other RNA
gctggactccagcctgggttggttttagagctagaaatagcaagttaaaataaggctagtccgttatcaacttgaaaaagtggcaccgagtcggtgctacgcccttcgagcatgtaggtcacggcccaggctggagttttt

SEQ ID NO: 118 - RNA, 141 nt, synthetic construct
  source 1..141: mol_type = other RNA
ctggactccagcctgggttggttttagagctagaaatagcaagttaaaataaggctagtccgttatcaacttgaaaaagtggcaccgagtcggtgcgacgacggagaccgccgtcgtcgacaagcccccaggctggatttt

SEQ ID NO: 119 - RNA, 141 nt, synthetic construct
  source 1..141: mol_type = other RNA
ctggactccagcctgggttggttttagagctagaaatagcaagttaaaataaggctagtccgttatcaacttgaaaaagtggcaccgagtcggtgcgacgacggcggtctccgtcgtcaggatcatcccaggctggatttt

SEQ ID NO: 120 - RNA, 141 nt, synthetic construct
  source 1..141: mol_type = other RNA
ctggactccagcctgggttggttttagagctagaaatagcaagttaaaataaggctagtccgttatcaacttgaaaaagtggcaccgagtcggtgcgacgacggagaccgccgtcgtcgacaagcccccaggctggagtcc

SEQ ID NO: 121 - RNA, 142 nt, synthetic construct
  source 1..142: mol_type = other RNA
ctggactccagcctgggttggttttagagctagaaatagcaagttaaaataaggctagtccgttatcaacttgaaaaagtggcaccgagtcggtgcggcgcatacgcccttcgagcatgtaggtcacggcccaggctggagt

SEQ ID NO: 122 - RNA, 142 nt, synthetic construct
  source 1..142: mol_type = other RNA
ctggactccagcctgggttggttttagagctagaaatagcaagttaaaataaggctagtccgttatcaacttgaaaaagtggcaccgagtcggtgcatgatcctgacgacggagaccgccgtcgtcgacaagcccccaggct

SEQ ID NO: 123 - RNA, 143 nt, synthetic construct
  source 1..143: mol_type = other RNA
ctggactccagcctgggttggttttagagctagaaatagcaagttaaaataaggctagtccgttatcaacttgaaaaagtggcaccgagtcggtgcgacgacggagaccgccgtcgtcgacaagcccccaggctggagttttt

SEQ ID NO: 124 - RNA, 143 nt, synthetic construct
  source 1..143: mol_type = other RNA
ctggactccagcctgggttggttttagagctagaaatagcaagttaaaataaggctagtccgttatcaacttgaaaaagtggcaccgagtcggtgcgacgacggcggtctccgtcgtcaggatcatcccaggctggagttttt

SEQ ID NO: 125 - RNA, 143 nt, synthetic construct
  source 1..143: mol_type = other RNA
ctggactccagcctgggttggttttagagctagaaatagcaagttaaaataaggctagtccgttatcaacttgaaaaagtggcaccgagtcggtgcgacgacggagaccgccgtcgtcgacaagcccccaggctggagtccag

SEQ ID NO: 126 - RNA, 144 nt, synthetic construct
  source 1..144: mol_type = other RNA
ctggactccagcctgggttggttttagagctagaaatagcaagttaaaataaggctagtccgttatcaacttgaaaaagtggcaccgagtcggtgcatgatcctgacgacggagaccgccgtcgtcgacaagcccccaggctgg

SEQ ID NO: 127 - RNA, 144 nt, synthetic construct
  source 1..144: mol_type = other RNA
gctggactccagcctgggttggttttagagctagaaatagcaagttaaaataaggctagtccgttatcaacttgaaaaagtggcaccgagtcggtgcgcatacgcccttcgagcatgtaggtcacggcccaggctggagttttt

SEQ ID NO: 128 - RNA, 145 nt, synthetic construct
  source 1..145: mol_type = other RNA
ctggactccagcctgggttggttttagagctagaaatagcaagttaaaataaggctagtccgttatcaacttgaaaaagtggcaccgagtcggtgcgacgacggagaccgccgtcgtcgacaagcccccaggctggagtcctttt

SEQ ID NO: 129 - RNA, 145 nt, synthetic construct
  source 1..145: mol_type = other RNA
ctggactccagcctgggttggttttagagctagaaatagcaagttaaaataaggctagtccgttatcaacttgaaaaagtggcaccgagtcggtgcatgatcctgacgacggagaccgccgtcgtcgacaagcccccaggctgga

SEQ ID NO: 130 - RNA, 145 nt, synthetic construct
  source 1..145: mol_type = other RNA
ctggactccagcctgggttggttttagagctagaaatagcaagttaaaataaggctagtccgttatcaacttgaaaaagtggcaccgagtcggtgcggcttgtcgacgacggcggtctccgtcgtcaggatcatcccaggctgga

SEQ ID NO: 131 - RNA, 146 nt, synthetic construct
  source 1..146: mol_type = other RNA
ctggactccagcctgggttggttttagagctagaaatagcaagttaaaataaggctagtccgttatcaacttgaaaaagtggcaccgagtcggtgcatgatcctgacgacggagaccgccgtcgtcgacaagcccccaggcttttt

SEQ ID NO: 132 - RNA, 147 nt, synthetic construct
  source 1..147: mol_type = other RNA
gctggactccagcctgggttggttttagagctagaaatagcaagttaaaataaggctagtccgttatcaacttgaaaaagtggcaccgagtcggtgcggcgcatacgcccttcgagcatgtaggtcacggcccaggctggagttttt

SEQ ID NO: 133 - RNA, 147 nt, synthetic construct
  source 1..147: mol_type = other RNA
ctggactccagcctgggttggttttagagctagaaatagcaagttaaaataaggctagtccgttatcaacttgaaaaagtggcaccgagtcggtgcgacgacggagaccgccgtcgtcgacaagcccccaggctggagtccagtttt

SEQ ID NO: 134 - RNA, 147 nt, synthetic construct
  source 1..147: mol_type = other RNA
ctggactccagcctgggttggttttagagctagaaatagcaagttaaaataaggctagtccgttatcaacttgaaaaagtggcaccgagtcggtgcatgatcctgacgacggagaccgccgtcgtcgacaagcccccaggctggagt

SEQ ID NO: 135 - RNA, 147 nt, synthetic construct
  source 1..147: mol_type = other RNA
ctggactccagcctgggttggttttagagctagaaatagcaagttaaaataaggctagtccgttatcaacttgaaaaagtggcaccgagtcggtgcggcttgtcgacgacggcggtctccgtcgtcaggatcatcccaggctggagt

SEQ ID NO: 136 - RNA, 148 nt, synthetic construct
  source 1..148: mol_type = other RNA
ctggactccagcctgggttggttttagagctagaaatagcaagttaaaataaggctagtccgttatcaacttgaaaaagtggcaccgagtcggtgcatgatcctgacgacggagaccgccgtcgtcgacaagcccccaggctggtttt

SEQ ID NO: 137 - RNA, 148 nt, synthetic construct
  source 1..148: mol_type = other RNA
ctggactccagcctgggttggttttagagctagaaatagcaagttaaaataaggctagtccgttatcaacttgaaaaagtggcaccgagtcggtgccttcgtggcgcatacgcccttcgagcatgtaggtcacggcccaggctggagt

SEQ ID NO: 138 - RNA, 149 nt, synthetic construct
  source 1..149: mol_type = other RNA
ctggactccagcctgggttggttttagagctagaaatagcaagttaaaataaggctagtccgttatcaacttgaaaaagtggcaccgagtcggtgcatgatcctgacgacggagaccgccgtcgtcgacaagcccccaggctggatttt

SEQ ID NO: 139 - RNA, 149 nt, synthetic construct
  source 1..149: mol_type = other RNA
ctggactccagcctgggttggttttagagctagaaatagcaagttaaaataaggctagtccgttatcaacttgaaaaagtggcaccgagtcggtgcggcttgtcgacgacggcggtctccgtcgtcaggatcatcccaggctggatttt

SEQ ID NO: 140 - RNA, 149 nt, synthetic construct
  source 1..149: mol_type = other RNA
ctggactccagcctgggttggttttagagctagaaatagcaagttaaaataaggctagtccgttatcaacttgaaaaagtggcaccgagtcggtgcatgatcctgacgacggagaccgccgtcgtcgacaagcccccaggctggagtcc

SEQ ID NO: 141 - RNA, 151 nt, synthetic construct
  source 1..151: mol_type = other RNA
ctggactccagcctgggttggttttagagctagaaatagcaagttaaaataaggctagtccgttatcaacttgaaaaagtggcaccgagtcggtgcatgatcctgacgacggagaccgccgtcgtcgacaagcccccaggctggagttttt

SEQ ID NO: 142 - RNA, 151 nt, synthetic construct
  source 1..151: mol_type = other RNA
ctggactccagcctgggttggttttagagctagaaatagcaagttaaaataaggctagtccgttatcaacttgaaaaagtggcaccgagtcggtgcggcttgtcgacgacggcggtctccgtcgtcaggatcatcccaggctggagttttt

SEQ ID NO: 143 - RNA, 151 nt, synthetic construct
  source 1..151: mol_type = other RNA
ctggactccagcctgggttggttttagagctagaaatagcaagttaaaataaggctagtccgttatcaacttgaaaaagtggcaccgagtcggtgcatgatcctgacgacggagaccgccgtcgtcgacaagcccccaggctggagtccag

SEQ ID NO: 144 - RNA, 153 nt, synthetic construct
  source 1..153: mol_type = other RNA
gctggactccagcctgggttggttttagagctagaaatagcaagttaaaataaggctagtccgttatcaacttgaaaaagtggcaccgagtcggtgccttcgtggcgcatacgcccttcgagcatgtaggtcacggcccaggctggagttttt

SEQ ID NO: 145 - RNA, 153 nt, synthetic construct
  source 1..153: mol_type = other RNA
ctggactccagcctgggttggttttagagctagaaatagcaagttaaaataaggctagtccgttatcaacttgaaaaagtggcaccgagtcggtgcatgatcctgacgacggagaccgccgtcgtcgacaagcccccaggctggagtcctttt

SEQ ID NO: 146 - RNA, 155 nt, synthetic construct
  source 1..155: mol_type = other RNA
ctggactccagcctgggttggttttagagctagaaatagcaagttaaaataaggctagtccgttatcaacttgaaaaagtggcaccgagtcggtgcatgatcctgacgacggagaccgccgtcgtcgacaagcccccaggctggagtccagtttt

SEQ ID NO: 147 - RNA, 17 nt, synthetic construct
  source 1..17: mol_type = other RNA
ggactccagcctgggtt

SEQ ID NO: 148 - RNA, 18 nt, synthetic construct
  source 1..18: mol_type = other RNA
tggactccagcctgggtt

SEQ ID NO: 149 - RNA, 19 nt, synthetic construct
  source 1..19: mol_type = other RNA
ctggactccagcctgggtt

SEQ ID NO: 150 - RNA, 20 nt, synthetic construct
  source 1..20: mol_type = other RNA
tctggactccagcctgggtt

SEQ ID NO: 151 - RNA, 21 nt, synthetic construct
  source 1..21: mol_type = other RNA
atctggactccagcctgggtt

SEQ ID NO: 152 - RNA, 22 nt, synthetic construct
  source 1..22: mol_type = other RNA
catctggactccagcctgggtt

SEQ ID NO: 153-157 - skipped (5 entries)

SEQ ID NO: 158 - RNA, 10 nt, synthetic construct
  source 1..10: mol_type = other RNA
ccaggctgga

SEQ ID NO: 159 - RNA, 11 nt, synthetic construct
  source 1..11: mol_type = other RNA
ccaggctggag

SEQ ID NO: 160 - RNA, 12 nt, synthetic construct
  source 1..12: mol_type = other RNA
ccaggctggagt

SEQ ID NO: 161 - RNA, 13 nt, synthetic construct
  source 1..13: mol_type = other RNA
ccaggctggagtg

SEQ ID NO: 162 - RNA, 14 nt, synthetic construct
  source 1..14: mol_type = other RNA
ccaggctggagtgc

SEQ ID NO: 163 - RNA, 15 nt, synthetic construct
  source 1..15: mol_type = other RNA
ccaggctggagtgca

SEQ ID NO: 164 - RNA, 16 nt, synthetic construct
  source 1..16: mol_type = other RNA
ccaggctggagtgcaa

SEQ ID NO: 165 - RNA, 17 nt, synthetic construct
  source 1..17: mol_type = other RNA
ccaggctggagtgcaat

SEQ ID NO: 166 - RNA, 18 nt, synthetic construct
  source 1..18: mol_type = other RNA
ccaggctggagtgcaatg

SEQ ID NO: 167 - RNA, 19 nt, synthetic construct
  source 1..19: mol_type = other RNA
ccaggctggagtgcaatgg

SEQ ID NO: 168 - RNA, 142 nt, synthetic construct
  source 1..142: mol_type = other RNA
tctggactccagcctgggttgttttagagctagaaatagcaagttaaaataaggctagtccgttatcaacttgaaaaagtggcaccgagtcggtgcatgatcctgacgacggagaccgccgtcgtcgacaagccccaggctg

SEQ ID NO: 169 - RNA, 144 nt, synthetic construct
  source 1..144: mol_type = other RNA
tctggactccagcctgggttgttttagagctagaaatagcaagttaaaataaggctagtccgttatcaacttgaaaaagtggcaccgagtcggtgcatgatcctgacgacggagaccgccgtcgtcgacaagccccaggctgga

SEQ ID NO: 170 - RNA, 146 nt, synthetic construct
  source 1..146: mol_type = other RNA
tctggactccagcctgggttgttttagagctagaaatagcaagttaaaataaggctagtccgttatcaacttgaaaaagtggcaccgagtcggtgcatgatcctgacgacggagaccgccgtcgtcgacaagccccaggctgtttt

SEQ ID NO: 171 - RNA, 146 nt, synthetic construct
  source 1..146: mol_type = other RNA
tctggactccagcctgggttgttttagagctagaaatagcaagttaaaataaggctagtccgttatcaacttgaaaaagtggcaccgagtcggtgcatgatcctgacgacggagaccgccgtcgtcgacaagccccaggctggagt

SEQ ID NO: 172 - RNA, 148 nt, synthetic construct
  source 1..148: mol_type = other RNA
tctggactccagcctgggttgttttagagctagaaatagcaagttaaaataaggctagtccgttatcaacttgaaaaagtggcaccgagtcggtgcatgatcctgacgacggagaccgccgtcgtcgacaagccccaggctggatttt

SEQ ID NO: 173 - RNA, 150 nt, synthetic construct
  source 1..150: mol_type = other RNA
tctggactccagcctgggttgttttagagctagaaatagcaagttaaaataaggctagtccgttatcaacttgaaaaagtggcaccgagtcggtgcatgatcctgacgacggagaccgccgtcgtcgacaagccccaggctggagttttt

SEQ ID NO: 174 - RNA, 17 nt, synthetic construct
  source 1..17: mol_type = other RNA
gtctctcagctggtaca

SEQ ID NO: 175 - RNA, 18 nt, synthetic construct
  source 1..18: mol_type = other RNA
agtctctcagctggtaca

SEQ ID NO: 176 - RNA, 19 nt, synthetic construct
  source 1..19: mol_type = other RNA
gagtctctcagctggtaca

SEQ ID NO: 177 - RNA, 20 nt, synthetic construct
  source 1..20: mol_type = other RNA
agagtctctcagctggtaca

SEQ ID NO: 178 - RNA, 21 nt, synthetic construct
  source 1..21: mol_type = other RNA
tagagtctctcagctggtaca

SEQ ID NO: 179 - RNA, 22 nt, synthetic construct
  source 1..22: mol_type = other RNA
ttagagtctctcagctggtaca

SEQ ID NO: 180-184 - skipped (5 entries)

SEQ ID NO: 185 - RNA, 10 nt, synthetic construct
  source 1..10: mol_type = other RNA
accagctgag

SEQ ID NO: 186 - RNA, 11 nt, synthetic construct
  source 1..11: mol_type = other RNA
accagctgaga

SEQ ID NO: 187 - RNA, 12 nt, synthetic construct
  source 1..12: mol_type = other RNA
accagctgagag

SEQ ID NO: 188 - RNA, 13 nt, synthetic construct
  source 1..13: mol_type = other RNA
accagctgagaga

SEQ ID NO: 189 - RNA, 14 nt, synthetic construct
  source 1..14: mol_type = other RNA
accagctgagagac

SEQ ID NO: 190 - RNA, 15 nt, synthetic construct
  source 1..15: mol_type = other RNA
accagctgagagact

SEQ ID NO: 191 - RNA, 16 nt, synthetic construct
  source 1..16: mol_type = other RNA
accagctgagagactc

SEQ ID NO: 192 - RNA, 17 nt, synthetic construct
  source 1..17: mol_type = other RNA
accagctgagagactct

SEQ ID NO: 193 - RNA, 18 nt, synthetic construct
  source 1..18: mol_type = other RNA
accagctgagagactcta

SEQ ID NO: 194 - RNA, 19 nt, synthetic construct
  source 1..19: mol_type = other RNA
accagctgagagactctaa

SEQ ID NO: 195 - RNA, 27 nt, synthetic construct
  source 1..27: mol_type = other RNA
cctacatgctcgaagggcgtatgcgcc

SEQ ID NO: 196 - RNA, 29 nt, synthetic construct
  source 1..29: mol_type = other RNA
acgacggcggtctccgtcgtcaggatcat

SEQ ID NO: 197 - RNA, 30 nt, synthetic construct
  source 1..30: mol_type = other RNA
acatgctcgaagggcgtatgcgccacgaag

SEQ ID NO: 198 - RNA, 33 nt, synthetic construct
  source 1..33: mol_type = other RNA
ccgtgacctacatgctcgaagggcgtatgcgcc

SEQ ID NO: 199 - RNA, 39 nt, synthetic construct
  source 1..39: mol_type = other RNA
ccgtgacctacatgctcgaagggcgtatgcgccacgaag

SEQ ID NO: 200 - RNA, 131 nt, synthetic construct
  source 1..131: mol_type = other RNA
agagtctctcagctggtacagttttagagctagaaatagcaagttaaaataaggctagtccgttatcaacttgaaaaagtggcaccgagtcggtgcacggcggtctccgtcgtcaggatcataccagctga

SEQ ID NO: 201 - RNA, 132 nt, synthetic construct
  source 1..132: mol_type = other RNA
agagtctctcagctggtacagttttagagctagaaatagcaagttaaaataaggctagtccgttatcaacttgaaaaagtggcaccgagtcggtgccctacatgctcgaagggcgtatgcgccaccagctga

SEQ ID NO: 202 - RNA, 134 nt, synthetic construct
  source 1..134: mol_type = other RNA
agagtctctcagctggtacagttttagagctagaaatagcaagttaaaataaggctagtccgttatcaacttgaaaaagtggcaccgagtcggtgcacgacggcggtctccgtcgtcaggatcataccagctga

SEQ ID NO: 203 - RNA, 135 nt, synthetic construct
  source 1..135: mol_type = other RNA
agagtctctcagctggtacagttttagagctagaaatagcaagttaaaataaggctagtccgttatcaacttgaaaaagtggcaccgagtcggtgcacggcggtctccgtcgtcaggatcataccagctgatttt

SEQ ID NO: 204 - RNA, 135 nt, synthetic construct
  source 1..135: mol_type = other RNA
agagtctctcagctggtacagttttagagctagaaatagcaagttaaaataaggctagtccgttatcaacttgaaaaagtggcaccgagtcggtgcacatgctcgaagggcgtatgcgccacgaagaccagctga

SEQ ID NO: 205 - RNA, 136 nt, synthetic construct
  source 1..136: mol_type = other RNA
agagtctctcagctggtacagttttagagctagaaatagcaagttaaaataaggctagtccgttatcaacttgaaaaagtggcaccgagtcggtgccctacatgctcgaagggcgtatgcgccaccagctgatttt

SEQ ID NO: 206 - RNA, 137 nt, synthetic construct
  source 1..137: mol_type = other RNA
agagtctctcagctggtacagttttagagctagaaatagcaagttaaaataaggctagtccgttatcaacttgaaaaagtggcaccgagtcggtgcgacgacggcggtctccgtcgtcaggatcataccagctgaga

SEQ ID NO: 207 - RNA, 138 nt, synthetic construct
  source 1..138: mol_type = other RNA
agagtctctcagctggtacagttttagagctagaaatagcaagttaaaataaggctagtccgttatcaacttgaaaaagtggcaccgagtcggtgcacgacggcggtctccgtcgtcaggatcataccagctgagaga

SEQ ID NO: 208 - RNA, 138 nt, synthetic construct
  source 1..138: mol_type = other RNA
agagtctctcagctggtacagttttagagctagaaatagcaagttaaaataaggctagtccgttatcaacttgaaaaagtggcaccgagtcggtgcccgtgacctacatgctcgaagggcgtatgcgccaccagctga

SEQ ID NO: 209 - RNA, 139 nt, synthetic construct
  source 1..139: mol_type = other RNA
agagtctctcagctggtacagttttagagctagaaatagcaagttaaaataaggctagtccgttatcaacttgaaaaagtggcaccgagtcggtgcacatgctcgaagggcgtatgcgccacgaagaccagctgatttt

SEQ ID NO: 210 - RNA, 139 nt, synthetic construct
  source 1..139: mol_type = other RNA
agagtctctcagctggtacagttttagagctagaaatagcaagttaaaataaggctagtccgttatcaacttgaaaaagtggcaccgagtcggtgcgacgacggcggtctccgtcgtcaggatcataccagctgagaga

SEQ ID NO: 211 - RNA, 139 nt, synthetic construct
  source 1..139: mol_type = other RNA
agagtctctcagctggtacagttttagagctagaaatagcaagttaaaataaggctagtccgttatcaacttgaaaaagtggcaccgagtcggtgcgacgacggagaccgccgtcgtcgacaagccaccagctgagaga

SEQ ID NO: 212 - RNA, 141 nt, synthetic construct
  source 1..141: mol_type = other RNA
agagtctctcagctggtacagttttagagctagaaatagcaagttaaaataaggctagtccgttatcaacttgaaaaagtggcaccgagtcggtgcgacgacggcggtctccgtcgtcaggatcataccagctgagatttt

SEQ ID NO: 213 - RNA, 141 nt, synthetic construct
  source 1..141: mol_type = other RNA
agagtctctcagctggtacagttttagagctagaaatagcaagttaaaataaggctagtccgttatcaacttgaaaaagtggcaccgagtcggtgcgacgacggcggtctccgtcgtcaggatcataccagctgagagact

SEQ ID NO: 214 - RNA, 142 nt, synthetic construct
  source 1..142: mol_type = other RNA
agagtctctcagctggtacagttttagagctagaaatagcaagttaaaataaggctagtccgttatcaacttgaaaaagtggcaccgagtcggtgcccgtgacctacatgctcgaagggcgtatgcgccaccagctgatttt

SEQ ID NO: 215 - RNA, 143 nt, synthetic construct
  source 1..143: mol_type = other RNA
agagtctctcagctggtacagttttagagctagaaatagcaagttaaaataaggctagtccgttatcaacttgaaaaagtggcaccgagtcggtgcgacgacggcggtctccgtcgtcaggatcataccagctgagagatttt

SEQ ID NO: 216 - RNA, 143 nt, synthetic construct
  source 1..143: mol_type = other RNA
agagtctctcagctggtacagttttagagctagaaatagcaagttaaaataaggctagtccgttatcaacttgaaaaagtggcaccgagtcggtgcgacgacggagaccgccgtcgtcgacaagccaccagctgagagatttt

SEQ ID NO: 217 - RNA, 143 nt, synthetic construct
  source 1..143: mol_type = other RNA
agagtctctcagctggtacagttttagagctagaaatagcaagttaaaataaggctagtccgttatcaacttgaaaaagtggcaccgagtcggtgcggcttgtcgacgacggcggtctccgtcgtcaggatcataccagctga

SEQ ID NO: 218 - RNA, 144 nt, synthetic construct
  source 1..144: mol_type = other RNA
agagtctctcagctggtacagttttagagctagaaatagcaagttaaaataaggctagtccgttatcaacttgaaaaagtggcaccgagtcggtgcccgtgacctacatgctcgaagggcgtatgcgccacgaagaccagctga

SEQ ID NO: 219 - RNA, 145 nt, synthetic construct
  source 1..145: mol_type = other RNA
agagtctctcagctggtacagttttagagctagaaatagcaagttaaaataaggctagtccgttatcaacttgaaaaagtggcaccgagtcggtgcgacgacggcggtctccgtcgtcaggatcataccagctgagagacttttt

SEQ ID NO: 220 - RNA, 145 nt, synthetic construct
  source 1..145: mol_type = other RNA
agagtctctcagctggtacagttttagagctagaaatagcaagttaaaataaggctagtccgttatcaacttgaaaaagtggcaccgagtcggtgcggcttgtcgacgacggcggtctccgtcgtcaggatcataccagctgaga

SEQ ID NO: 221 - RNA, 147 nt, synthetic construct
  source 1..147: mol_type = other RNA
agagtctctcagctggtacagttttagagctagaaatagcaagttaaaataaggctagtccgttatcaacttgaaaaagtggcaccgagtcggtgcggcttgtcgacgacggcggtctccgtcgtcaggatcataccagctgagaga

SEQ ID NO: 222 - RNA, 147 nt, synthetic construct
  source 1..147: mol_type = other RNA
agagtctctcagctggtacagttttagagctagaaatagcaagttaaaataaggctagtccgttatcaacttgaaaaagtggcaccgagtcggtgcggcttgtcgacgacggcggtctccgtcgtcaggatcataccagctgatttt

SEQ ID NO: 223 - RNA, 148 nt, synthetic construct
  source 1..148: mol_type = other RNA
agagtctctcagctggtacagttttagagctagaaatagcaagttaaaataaggctagtccgttatcaacttgaaaaagtggcaccgagtcggtgcccgtgacctacatgctcgaagggcgtatgcgccacgaagaccagctgatttt

SEQ ID NO: 224 - RNA, 149 nt, synthetic construct
  source 1..149: mol_type = other RNA
agagtctctcagctggtacagttttagagctagaaatagcaagttaaaataaggctagtccgttatcaacttgaaaaagtggcaccgagtcggtgcggcttgtcgacgacggcggtctccgtcgtcaggatcataccagctgagatttt

SEQ ID NO: 225 - RNA, 149 nt, synthetic construct
  source 1..149: mol_type = other RNA
agagtctctcagctggtacagttttagagctagaaatagcaagttaaaataaggctagtccgttatcaacttgaaaaagtggcaccgagtcggtgcggcttgtcgacgacggcggtctccgtcgtcaggatcataccagctgagagact

SEQ ID NO: 226 - RNA, 151 nt, synthetic construct
  source 1..151: mol_type = other RNA
agagtctctcagctggtacagttttagagctagaaatagcaagttaaaataaggctagtccgttatcaacttgaaaaagtggcaccgagtcggtgcggcttgtcgacgacggcggtctccgtcgtcaggatcataccagctgagagatttt

SEQ ID NO: 227 - RNA, 151 nt, synthetic construct
  source 1..151: mol_type = other RNA
agagtctctcagctggtacagttttagagctagaaatagcaagttaaaataaggctagtccgttatcaacttgaaaaagtggcaccgagtcggtgcatgatcctgacgacggagaccgccgtcgtcgacaagccaccagctgagagatttt

SEQ ID NO: 228 - RNA, 153 nt, synthetic construct
  source 1..153: mol_type = other RNA
agagtctctcagctggtacagttttagagctagaaatagcaagttaaaataaggctagtccgttatcaacttgaaaaagtggcaccgagtcggtgcggcttgtcgacgacggcggtctccgtcgtcaggatcataccagctgagagacttttt

SEQ ID NO: 229 - RNA, 155 nt, synthetic construct
  source 1..155: mol_type = other RNA
agagtctctcagctggtacagttttagagctagaaatagcaagttaaaataaggctagtccgttatcaacttgaaaaagtggcaccgagtcggtgcatgatcctgacgacggagaccgccgtcgtcgacaagccaccagctgagagatttttttt

SEQ ID NO: 230 - RNA, 17 nt, synthetic construct
  source 1..17: mol_type = other RNA
tggtacacggcagggtc

SEQ ID NO: 231 - RNA, 18 nt, synthetic construct
  source 1..18: mol_type = other RNA
ctggtacacggcagggtc

SEQ ID NO: 232 - RNA, 19 nt, synthetic construct
  source 1..19: mol_type = other RNA
gctggtacacggcagggtc

SEQ ID NO: 233 - RNA, 20 nt, synthetic construct
  source 1..20: mol_type = other RNA
agctggtacacggcagggtc

SEQ ID NO: 234 - RNA, 21 nt, synthetic construct
  source 1..21: mol_type = other RNA
cagctggtacacggcagggtc

SEQ ID NO: 235 - RNA, 22 nt, synthetic construct
  source 1..22: mol_type = other RNA
tcagctggtacacggcagggtc

SEQ ID NO: 236-240 - skipped (5 entries)

SEQ ID NO: 241 - RNA, 10 nt, synthetic construct
  source 1..10: mol_type = other RNA
cctgccgtgt

SEQ ID NO: 242 - RNA, 11 nt, synthetic construct
  source 1..11: mol_type = other RNA
cctgccgtgta

SEQ ID NO: 243 - RNA, 12 nt, synthetic construct
  source 1..12: mol_type = other RNA
cctgccgtgtac

SEQ ID NO: 244 - RNA, 13 nt, synthetic construct
  source 1..13: mol_type = other RNA
cctgccgtgtacc

SEQ ID NO: 245 - RNA, 14 nt, synthetic construct
  source 1..14: mol_type = other RNA
cctgccgtgtacca

SEQ ID NO: 246 - RNA, 15 nt, synthetic construct
  source 1..15: mol_type = other RNA
cctgccgtgtaccag

SEQ ID NO: 247 - RNA, 16 nt, synthetic construct
  source 1..16: mol_type = other RNA
cctgccgtgtaccagc

SEQ ID NO: 248 - RNA, 17 nt, synthetic construct
  source 1..17: mol_type = other RNA
cctgccgtgtaccagct

SEQ ID NO: 249 - RNA, 18 nt, synthetic construct
  source 1..18: mol_type = other RNA
cctgccgtgtaccagctg

SEQ ID NO: 250 - RNA, 19 nt, synthetic construct
  source 1..19: mol_type = other RNA
cctgccgtgtaccagctga

SEQ ID NO: 251 - RNA, 142 nt, synthetic construct
  source 1..142: mol_type = other RNA
agctggtacacggcagggtcgttttagagctagaaatagcaagttaaaataaggctagtccgttatcaacttgaaaaagtggcaccgagtcggtgcggcttgtcgacgacggcggtctccgtcgtcaggatcatcctgccgt

SEQ ID NO: 252 - RNA, 144 nt, synthetic construct
  source 1..144: mol_type = other RNA
agctggtacacggcagggtcgttttagagctagaaatagcaagttaaaataaggctagtccgttatcaacttgaaaaagtggcaccgagtcggtgcggcttgtcgacgacggcggtctccgtcgtcaggatcatcctgccgtgt

SEQ ID NO: 253 - RNA, 146 nt, synthetic construct
  source 1..146: mol_type = other RNA
agctggtacacggcagggtcgttttagagctagaaatagcaagttaaaataaggctagtccgttatcaacttgaaaaagtggcaccgagtcggtgcggcttgtcgacgacggcggtctccgtcgtcaggatcatcctgccgttttt

SEQ ID NO: 254 - RNA, 146 nt, synthetic construct
  source 1..146: mol_type = other RNA
agctggtacacggcagggtcgttttagagctagaaatagcaagttaaaataaggctagtccgttatcaacttgaaaaagtggcaccgagtcggtgcggcttgtcgacgacggcggtctccgtcgtcaggatcatcctgccgtgtac

SEQ ID NO: 255 - RNA, 148 nt, synthetic construct
  source 1..148: mol_type = other RNA
agctggtacacggcagggtcgttttagagctagaaatagcaagttaaaataaggctagtccgttatcaacttgaaaaagtggcaccgagtcggtgcggcttgtcgacgacggcggtctccgtcgtcaggatcatcctgccgtgttttt

SEQ ID NO: 256 - RNA, 150 nt, synthetic construct
  source 1..150: mol_type = other RNA
agctggtacacggcagggtcgttttagagctagaaatagcaagttaaaataaggctagtccgttatcaacttgaaaaagtggcaccgagtcggtgcggcttgtcgacgacggcggtctccgtcgtcaggatcatcctgccgtgtactttt

SEQ ID NO: 257 - RNA, 17 nt, synthetic construct
  source 1..17: mol_type = other RNA
tgtgggacaagaggatc

SEQ ID NO: 258 - RNA, 18 nt, synthetic construct
  source 1..18: mol_type = other RNA
ctgtgggacaagaggatc

SEQ ID NO: 259 - RNA, 19 nt, synthetic construct
  source 1..19: mol_type = other RNA
tctgtgggacaagaggatc

SEQ ID NO: 260 - RNA, 20 nt, synthetic construct
  source 1..20: mol_type = other RNA
atctgtgggacaagaggatc

SEQ ID NO: 261 - RNA, 21 nt, synthetic construct
  source 1..21: mol_type = other RNA
tatctgtgggacaagaggatc

SEQ ID NO: 262 - RNA, 22 nt, synthetic construct
  source 1..22: mol_type = other RNA
atatctgtgggacaagaggatc

SEQ ID NO: 263-267 - skipped (5 entries)

SEQ ID NO: 268 - RNA, 10 nt, synthetic construct
  source 1..10: mol_type = other RNA
cctcttgtcc

SEQ ID NO: 269 - RNA, 11 nt, synthetic construct
  source 1..11: mol_type = other RNA
cctcttgtccc

SEQ ID NO: 270 - RNA, 12 nt, synthetic construct
  source 1..12: mol_type = other RNA
cctcttgtccca

SEQ ID NO: 271 - RNA, 13 nt, synthetic construct
  source 1..13: mol_type = other RNA
cctcttgtcccac

SEQ ID NO: 272 - RNA, 14 nt, synthetic construct
  source 1..14: mol_type = other RNA
cctcttgtcccaca

SEQ ID NO: 273 - RNA, 15 nt, synthetic construct
  source 1..15: mol_type = other RNA
cctcttgtcccacag

SEQ ID NO: 274 - RNA, 16 nt, synthetic construct
  source 1..16: mol_type = other RNA
cctcttgtcccacaga

SEQ ID NO: 275 - RNA, 17 nt, synthetic construct
  source 1..17: mol_type = other RNA
cctcttgtcccacagat

SEQ ID NO: 276 - RNA, 18 nt, synthetic construct
  source 1..18: mol_type = other RNA
cctcttgtcccacagata

SEQ ID NO: 277 - RNA, 19 nt, synthetic construct
  source 1..19: mol_type = other RNA
cctcttgtcccacagatat

SEQ ID NO: 278 - RNA, 143 nt, synthetic construct
  source 1..143: mol_type = other RNA
atctgtgggacaagaggatcgttttagagctagaaatagcaagttaaaataaggctagtccgttatcaacttgaaaaagtggcaccgagtcggtgcggcttgtcgacgacggcggtctccgtcgtcaggatcatcctcttgtc

SEQ ID NO: 279 - RNA, 145 nt, synthetic construct
  source 1..145: mol_type = other RNA
atctgtgggacaagaggatcgttttagagctagaaatagcaagttaaaataaggctagtccgttatcaacttgaaaaagtggcaccgagtcggtgcggcttgtcgacgacggcggtctccgtcgtcaggatcatcctcttgtccc

SEQ ID NO: 280 - RNA, 147 nt, synthetic construct
  source 1..147: mol_type = other RNA
atctgtgggacaagaggatcgttttagagctagaaatagcaagttaaaataaggctagtccgttatcaacttgaaaaagtggcaccgagtcggtgcggcttgtcgacgacggcggtctccgtcgtcaggatcatcctcttgtctttt

SEQ ID NO: 281 - RNA, 147 nt, synthetic construct
  source 1..147: mol_type = other RNA
atctgtgggacaagaggatcgttttagagctagaaatagcaagttaaaataaggctagtccgttatcaacttgaaaaagtggcaccgagtcggtgcggcttgtcgacgacggcggtctccgtcgtcaggatcatcctcttgtcccac

SEQ ID NO: 282 - RNA, 149 nt, synthetic construct
  source 1..149: mol_type = other RNA
atctgtgggacaagaggatcgttttagagctagaaatagcaagttaaaataaggctagtccgttatcaacttgaaaaagtggcaccgagtcggtgcggcttgtcgacgacggcggtctccgtcgtcaggatcatcctcttgtccctttt

SEQ ID NO: 283 - RNA, 151 nt, synthetic construct
  source 1..151: mol_type = other RNA
atctgtgggacaagaggatcgttttagagctagaaatagcaagttaaaataaggctagtccgttatcaacttgaaaaagtggcaccgagtcggtgcggcttgtcgacgacggcggtctccgtcgtcaggatcatcctcttgtcccactttt

SEQ ID NO: 284 - RNA, 17 nt, synthetic construct
  source 1..17: mol_type = other RNA
tcagctggtacacggca

SEQ ID NO: 285 - RNA, 18 nt, synthetic construct
  source 1..18: mol_type = other RNA
ctcagctggtacacggca

SEQ ID NO: 286 - RNA, 19 nt, synthetic construct
  source 1..19: mol_type = other RNA
tctcagctggtacacggca

SEQ ID NO: 287 - RNA, 20 nt, synthetic construct
  source 1..20: mol_type = other RNA
ctctcagctggtacacggca

SEQ ID NO: 288 - RNA, 21 nt, synthetic construct
  source 1..21: mol_type = other RNA
tctctcagctggtacacggca

SEQ ID NO: 289 - RNA, 22 nt, synthetic construct
  source 1..22: mol_type = other RNA
gtctctcagctggtacacggca

SEQ ID NO: 290-294 - skipped (5 entries)

SEQ ID NO: 295 - RNA, 10 nt, synthetic construct
  source 1..10: mol_type = other RNA
cgtgtaccag

SEQ ID NO: 296 - RNA, 11 nt, synthetic construct
  source 1..11: mol_type = other RNA
cgtgtaccagc

SEQ ID NO: 297 - RNA, 12 nt, synthetic construct
  source 1..12: mol_type = other RNA
cgtgtaccagct

SEQ ID NO: 298 - RNA, 13 nt, synthetic construct
  source 1..13: mol_type = other RNA
cgtgtaccagctg

SEQ ID NO: 299 - RNA, 14 nt, synthetic construct
  source 1..14: mol_type = other RNA
cgtgtaccagctga

SEQ ID NO: 300 - RNA, 15 nt, synthetic construct
  source 1..15: mol_type = other RNA
cgtgtaccagctgag

SEQ ID NO: 301 - RNA, 16 nt, synthetic construct
  source 1..16: mol_type = other RNA
cgtgtaccagctgaga

SEQ ID NO: 302 - RNA, 17 nt, synthetic construct
  source 1..17: mol_type = other RNA
cgtgtaccagctgagag

SEQ ID NO: 303 - RNA, 18 nt, synthetic construct
  source 1..18: mol_type = other RNA
cgtgtaccagctgagaga

SEQ ID NO: 304 - RNA, 19 nt, synthetic construct
  source 1..19: mol_type = other RNA
cgtgtaccagctgagagac

SEQ ID NO: 305 - RNA, 142 nt, synthetic construct
  source 1..142: mol_type = other RNA
ctctcagctggtacacggcagttttagagctagaaatagcaagttaaaataaggctagtccgttatcaacttgaaaaagtggcaccgagtcggtgcggcttgtcgacgacggcggtctccgtcgtcaggatcatcgtgtacc

SEQ ID NO: 306 - RNA, 144 nt, synthetic construct
  source 1..144: mol_type = other RNA
ctctcagctggtacacggcagttttagagctagaaatagcaagttaaaataaggctagtccgttatcaacttgaaaaagtggcaccgagtcggtgcggcttgtcgacgacggcggtctccgtcgtcaggatcatcgtgtaccag

SEQ ID NO: 307 - RNA, 146 nt, synthetic construct
  source 1..146: mol_type = other RNA
ctctcagctggtacacggcagttttagagctagaaatagcaagttaaaataaggctagtccgttatcaacttgaaaaagtggcaccgagtcggtgcggcttgtcgacgacggcggtctccgtcgtcaggatcatcgtgtacctttt

SEQ ID NO: 308 - RNA, 146 nt, synthetic construct
  source 1..146: mol_type = other RNA
ctctcagctggtacacggcagttttagagctagaaatagcaagttaaaataaggctagtccgttatcaacttgaaaaagtggcaccgagtcggtgcggcttgtcgacgacggcggtctccgtcgtcaggatcatcgtgtaccagct

SEQ ID NO: 309 - RNA, 148 nt, synthetic construct
  source 1..148: mol_type = other RNA
ctctcagctggtacacggcagttttagagctagaaatagcaagttaaaataaggctagtccgttatcaacttgaaaaagtggcaccgagtcggtgcggcttgtcgacgacggcggtctccgtcgtcaggatcatcgtgtaccagtttt

SEQ ID NO: 310 - RNA, 150 nt, synthetic construct
  source 1..150: mol_type = other RNA
ctctcagctggtacacggcagttttagagctagaaatagcaagttaaaataaggctagtccgttatcaacttgaaaaagtggcaccgagtcggtgcggcttgtcgacgacggcggtctccgtcgtcaggatcatcgtgtaccagcttttt

SEQ ID NO: 311 - RNA, 17 nt, synthetic construct
  source 1..17: mol_type = other RNA
gatatctgtgggacaag

SEQ ID NO: 312 - RNA, 18 nt, synthetic construct
  source 1..18: mol_type = other RNA
ggatatctgtgggacaag

SEQ ID NO: 313 - RNA, 19 nt, synthetic construct
  source 1..19: mol_type = other RNA
tggatatctgtgggacaag

SEQ ID NO: 314 - RNA, 20 nt, synthetic construct
  source 1..20: mol_type = other RNA
ctggatatctgtgggacaag

SEQ ID NO: 315 - RNA, 21 nt, synthetic construct
  source 1..21: mol_type = other RNA
tctggatatctgtgggacaag

SEQ ID NO: 316 - RNA, 22 nt, synthetic construct
  source 1..22: mol_type = other RNA
ttctggatatctgtgggacaag

SEQ ID NO: 317-321 - skipped (5 entries)

SEQ ID NO: 322 - RNA, 10 nt, synthetic construct
  source 1..10: mol_type = other RNA
gtcccacaga

SEQ ID NO: 323 - RNA, 11 nt, synthetic construct
  source 1..11: mol_type = other RNA
gtcccacagat

SEQ ID NO: 324 - RNA, 12 nt, synthetic construct
  source 1..12: mol_type = other RNA
gtcccacagata

SEQ ID NO: 325 - RNA, 13 nt, synthetic construct
  source 1..13: mol_type = other RNA
gtcccacagatat

SEQ ID NO: 326 - RNA, 14 nt, synthetic construct
  source 1..14: mol_type = other RNA
gtcccacagatatc

SEQ ID NO: 327 - RNA, 15 nt, synthetic construct
  source 1..15: mol_type = other RNA
gtcccacagatatcc

SEQ ID NO: 328 - RNA, 16 nt, synthetic construct
  source 1..16: mol_type = other RNA
gtcccacagatatcca

SEQ ID NO: 329 - RNA, 17 nt, synthetic construct
  source 1..17: mol_type = other RNA
gtcccacagatatccag

SEQ ID NO: 330 - RNA, 18 nt, synthetic construct
  source 1..18: mol_type = other RNA
gtcccacagatatccaga

SEQ ID NO: 331 - RNA, 19 nt, synthetic construct
  source 1..19: mol_type = other RNA
gtcccacagatatccagaa

SEQ ID NO: 332 - RNA, 143 nt, synthetic construct
  source 1..143: mol_type = other RNA
ctggatatctgtgggacaaggttttagagctagaaatagcaagttaaaataaggctagtccgttatcaacttgaaaaagtggcaccgagtcggtgcggcttgtcgacgacggcggtctccgtcgtcaggatcatgtcccacag

SEQ ID NO: 333 - RNA, 145 nt, synthetic construct
  source 1..145: mol_type = other RNA
ctggatatctgtgggacaaggttttagagctagaaatagcaagttaaaataaggctagtccgttatcaacttgaaaaagtggcaccgagtcggtgcggcttgtcgacgacggcggtctccgtcgtcaggatcatgtcccacagat

SEQ ID NO: 334 - RNA, 147 nt, synthetic construct
  source 1..147: mol_type = other RNA
ctggatatctgtgggacaaggttttagagctagaaatagcaagttaaaataaggctagtccgttatcaacttgaaaaagtggcaccgagtcggtgcggcttgtcgacgacggcggtctccgtcgtcaggatcatgtcccacagtttt

SEQ ID NO: 335 - RNA, 147 nt, synthetic construct
  source 1..147: mol_type = other RNA
ctggatatctgtgggacaaggttttagagctagaaatagcaagttaaaataaggctagtccgttatcaacttgaaaaagtggcaccgagtcggtgcggcttgtcgacgacggcggtctccgtcgtcaggatcatgtcccacagatat

SEQ ID NO: 336 - RNA, 149 nt, synthetic construct
  source 1..149: mol_type = other RNA
ctggatatctgtgggacaaggttttagagctagaaatagcaagttaaaataaggctagtccgttatcaacttgaaaaagtggcaccgagtcggtgcggcttgtcgacgacggcggtctccgtcgtcaggatcatgtcccacagattttt

SEQ ID NO: 337 - RNA, 151 nt, synthetic construct
  source 1..151: mol_type = other RNA
ctggatatctgtgggacaaggttttagagctagaaatagcaagttaaaataaggctagtccgttatcaacttgaaaaagtggcaccgagtcggtgcggcttgtcgacgacggcggtctccgtcgtcaggatcatgtcccacagatattttt

SEQ ID NO: 338 - RNA, 17 nt, synthetic construct
  source 1..17: mol_type = other RNA
aatcaaaatcggtgaat

SEQ ID NO: 339 - RNA, 18 nt, synthetic construct
  source 1..18: mol_type = other RNA
gaatcaaaatcggtgaat

SEQ ID NO: 340 - RNA, 19 nt, synthetic construct
  source 1..19: mol_type = other RNA
agaatcaaaatcggtgaat

SEQ ID NO: 341 - RNA, 20 nt, synthetic construct
  source 1..20: mol_type = other RNA
gagaatcaaaatcggtgaat

SEQ ID NO: 342 - RNA, 21 nt, synthetic construct
  source 1..21: mol_type = other RNA
tgagaatcaaaatcggtgaat

SEQ ID NO: 343 - RNA, 22 nt, synthetic construct
  source 1..22: mol_type = other RNA
ttgagaatcaaaatcggtgaat

SEQ ID NO: 344-348 - skipped (5 entries)

SEQ ID NO: 349 - RNA, 10 nt, synthetic construct
  source 1..10: mol_type = other RNA
caccgatttt

SEQ ID NO: 350 - RNA, 11 nt, synthetic construct
  source 1..11: mol_type = other RNA
caccgattttg

SEQ ID NO: 351 - RNA, 12 nt, synthetic construct
  source 1..12: mol_type = other RNA
caccgattttga

SEQ ID NO: 352 - RNA, 13 nt, synthetic construct
  source 1..13: mol_type = other RNA
caccgattttgat

SEQ ID NO: 353 - RNA, 14 nt, synthetic construct
  source 1..14: mol_type = other RNA
caccgattttgatt

SEQ ID NO: 354 - RNA, 15 nt, synthetic construct
  source 1..15: mol_type = other RNA
caccgattttgattc

SEQ ID NO: 355 - RNA, 16 nt, synthetic construct
  source 1..16: mol_type = other RNA
caccgattttgattct

SEQ ID NO: 356 - RNA, 17 nt, synthetic construct
  source 1..17: mol_type = other RNA
caccgattttgattctc

SEQ ID NO: 357 - RNA, 18 nt, synthetic construct
  source 1..18: mol_type = other RNA
caccgattttgattctca

SEQ ID NO: 358 - RNA, 19 nt, synthetic construct
  source 1..19: mol_type = other RNA
caccgattttgattctcaa

SEQ ID NO: 359 - RNA, 145 nt, synthetic construct
  source 1..145: mol_type = other RNA
gagaatcaaaatcggtgaatgttttagagctagaaatagcaagttaaaataaggctagtccgttatcaacttgaaaaagtggcaccgagtcggtgcggcttgtcgacgacggcggtctccgtcgtcaggatcatcaccgattttg

SEQ ID NO: 360 - RNA, 148 nt, synthetic construct
  source 1..148: mol_type = other RNA
gagaatcaaaatcggtgaatgttttagagctagaaatagcaagttaaaataaggctagtccgttatcaacttgaaaaagtggcaccgagtcggtgcggcttgtcgacgacggcggtctccgtcgtcaggatcatcaccgattttgatt

SEQ ID NO: 361 - RNA, 149 nt, synthetic construct
  source 1..149: mol_type = other RNA
gagaatcaaaatcggtgaatgttttagagctagaaatagcaagttaaaataaggctagtccgttatcaacttgaaaaagtggcaccgagtcggtgcggcttgtcgacgacggcggtctccgtcgtcaggatcatcaccgattttgtttt

SEQ ID NO: 362 - RNA, 150 nt, synthetic construct
  source 1..150: mol_type = other RNA
gagaatcaaaatcggtgaatgttttagagctagaaatagcaagttaaaataaggctagtccgttatcaacttgaaaaagtggcaccgagtcggtgcggcttgtcgacgacggcggtctccgtcgtcaggatcatcaccgattttgattct

SEQ ID NO: 363 - RNA, 152 nt, synthetic construct
  source 1..152: mol_type = other RNA
gagaatcaaaatcggtgaatgttttagagctagaaatagcaagttaaaataaggctagtccgttatcaacttgaaaaagtggcaccgagtcggtgcggcttgtcgacgacggcggtctccgtcgtcaggatcatcaccgattttgatttttt

SEQ ID NO: 364 - RNA, 154 nt, synthetic construct
  source 1..154: mol_type = other RNA
gagaatcaaaatcggtgaatgttttagagctagaaatagcaagttaaaataaggctagtccgttatcaacttgaaaaagtggcaccgagtcggtgcggcttgtcgacgacggcggtctccgtcgtcaggatcatcaccgattttgattcttttt

SEQ ID NO: 365 - RNA, 17 nt, synthetic construct
  source 1..17: mol_type = other RNA
ggtacacggcagggtca

SEQ ID NO: 366 - RNA, 18 nt, synthetic construct
  source 1..18: mol_type = other RNA
tggtacacggcagggtca

SEQ ID NO: 367 - RNA, 19 nt, synthetic construct
  source 1..19: mol_type = other RNA
ctggtacacggcagggtca

SEQ ID NO: 368 - RNA, 20 nt, synthetic construct
  source 1..20: mol_type = other RNA
gctggtacacggcagggtca

SEQ ID NO: 369 - RNA, 21 nt, synthetic construct
  source 1..21: mol_type = other RNA
agctggtacacggcagggtca

SEQ ID NO: 370 - RNA, 22 nt, synthetic construct
  source 1..22: mol_type = other RNA
cagctggtacacggcagggtca

SEQ ID NO: 371-375 - skipped (5 entries)

SEQ ID NO: 376 - RNA, 10 nt, synthetic construct
  source 1..10: mol_type = other RNA
ccctgccgtg

SEQ ID NO: 377 - RNA, 11 nt, synthetic construct
  source 1..11: mol_type = other RNA
ccctgccgtgt

SEQ ID NO: 378 - RNA, 12 nt, synthetic construct
  source 1..12: mol_type = other RNA
ccctgccgtgta

SEQ ID NO: 379 - RNA, 13 nt, synthetic construct
  source 1..13: mol_type = other RNA
ccctgccgtgtac

SEQ ID NO: 380 - RNA, 14 nt, synthetic construct
  source 1..14: mol_type = other RNA
ccctgccgtgtacc

SEQ ID NO: 381 - RNA, 15 nt, synthetic construct
  source 1..15: mol_type = other RNA
ccctgccgtgtacca

SEQ ID NO: 382 - RNA, 16 nt, synthetic construct
  source 1..16: mol_type = other RNA
ccctgccgtgtaccag

SEQ ID NO: 383 - RNA, 17 nt, synthetic construct
  source 1..17: mol_type = other RNA
ccctgccgtgtaccagc

SEQ ID NO: 384 - RNA, 18 nt, synthetic construct
  source 1..18: mol_type = other RNA
ccctgccgtgtaccagct

SEQ ID NO: 385 - RNA, 19 nt, synthetic construct
  source 1..19: mol_type = other RNA
ccctgccgtgtaccagctg

SEQ ID NO: 386 - RNA, 131 nt, synthetic construct
  source 1..131: mol_type = other RNA
gctggtacacggcagggtcagttttagagctagaaatagcaagttaaaataaggctagtccgttatcaacttgaaaaagtggcaccgagtcggtgcacgacggcggtctccgtcgtcaggatcatccctgc

SEQ ID NO: 387 - RNA, 133 nt, synthetic construct
  source 1..133: mol_type = other RNA
gctggtacacggcagggtcagttttagagctagaaatagcaagttaaaataaggctagtccgttatcaacttgaaaaagtggcaccgagtcggtgcgacgacggcggtctccgtcgtcaggatcatccctgcc

SEQ ID NO: 388 - RNA, 135 nt, synthetic construct
  source 1..135: mol_type = other RNA
gctggtacacggcagggtcagttttagagctagaaatagcaagttaaaataaggctagtccgttatcaacttgaaaaagtggcaccgagtcggtgcacgacggcggtctccgtcgtcaggatcatccctgccgtg

SEQ ID NO: 389 - RNA, 135 nt, synthetic construct
  source 1..135: mol_type = other RNA
gctggtacacggcagggtcagttttagagctagaaatagcaagttaaaataaggctagtccgttatcaacttgaaaaagtggcaccgagtcggtgcgacgacggcggtctccgtcgtcaggatcatccctgccgt

SEQ ID NO: 390 - RNA, 137 nt, synthetic construct
  source 1..137: mol_type = other RNA
gctggtacacggcagggtcagttttagagctagaaatagcaagttaaaataaggctagtccgttatcaacttgaaaaagtggcaccgagtcggtgcgacgacggcggtctccgtcgtcaggatcatccctgcctttt

SEQ ID NO: 391 - RNA, 137 nt, synthetic construct
  source 1..137: mol_type = other RNA
gctggtacacggcagggtcagttttagagctagaaatagcaagttaaaataaggctagtccgttatcaacttgaaaaagtggcaccgagtcggtgcgacgacggcggtctccgtcgtcaggatcatccctgccgtgt

SEQ ID NO: 392 - RNA, 139 nt, synthetic construct
  source 1..139: mol_type = other RNA
gctggtacacggcagggtcagttttagagctagaaatagcaagttaaaataaggctagtccgttatcaacttgaaaaagtggcaccgagtcggtgcgacgacggcggtctccgtcgtcaggatcatccctgccgttttt

SEQ ID NO: 393 - RNA, 139 nt, synthetic construct
  source 1..139: mol_type = other RNA
gctggtacacggcagggtcagttttagagctagaaatagcaagttaaaataaggctagtccgttatcaacttgaaaaagtggcaccgagtcggtgcgacgacggcggtctccgtcgtcaggatcatccctgccgtgtac

SEQ ID NO: 394 - RNA, 140 nt, synthetic construct
  source 1..140: mol_type = other RNA
gctggtacacggcagggtcagttttagagctagaaatagcaagttaaaataaggctagtccgttatcaacttgaaaaagtggcaccgagtcggtgcggcttgtcgacgacggcggtctccgtcgtcaggatcatccctgc

SEQ ID NO: 395 - RNA, 141 nt, synthetic construct
  source 1..141: mol_type = other RNA
gctggtacacggcagggtcagttttagagctagaaatagcaagttaaaataaggctagtccgttatcaacttgaaaaagtggcaccgagtcggtgcgacgacggcggtctccgtcgtcaggatcatccctgccgtgttttt

SEQ ID NO: 396 - RNA, 141 nt, synthetic construct
  source 1..141: mol_type = other RNA
gctggtacacggcagggtcagttttagagctagaaatagcaagttaaaataaggctagtccgttatcaacttgaaaaagtggcaccgagtcggtgcggcttgtcgacgacggcggtctccgtcgtcaggatcatccctgcc

SEQ ID NO: 397 - RNA, 142 nt, synthetic construct
  source 1..142: mol_type = other RNA
gctggtacacggcagggtcagttttagagctagaaatagcaagttaaaataaggctagtccgttatcaacttgaaaaagtggcaccgagtcggtgcggcttgtcgacgacggcggtctccgtcgtcaggatcatccctgccg

SEQ ID NO: 398 - RNA, 143 nt, synthetic construct
  source 1..143: mol_type = other RNA
gctggtacacggcagggtcagttttagagctagaaatagcaagttaaaataaggctagtccgttatcaacttgaaaaagtggcaccgagtcggtgcgacgacggcggtctccgtcgtcaggatcatccctgccgtgtactttt

SEQ ID NO: 399 - RNA, 143 nt, synthetic construct
  source 1..143: mol_type = other RNA
gctggtacacggcagggtcagttttagagctagaaatagcaagttaaaataaggctagtccgttatcaacttgaaaaagtggcaccgagtcggtgcggcttgtcgacgacggcggtctccgtcgtcaggatcatccctgccgt

SEQ ID NO: 400 - RNA, 144 nt, synthetic construct
  source 1..144: mol_type = other RNA
gctggtacacggcagggtcagttttagagctagaaatagcaagttaaaataaggctagtccgttatcaacttgaaaaagtggcaccgagtcggtgcggcttgtcgacgacggcggtctccgtcgtcaggatcatccctgccgtg

SEQ ID NO: 401 - RNA, 145 nt, synthetic construct
  source 1..145: mol_type = other RNA
gctggtacacggcagggtcagttttagagctagaaatagcaagttaaaataaggctagtccgttatcaacttgaaaaagtggcaccgagtcggtgcggcttgtcgacgacggcggtctccgtcgtcaggatcatccctgcctttt

SEQ ID NO: 402 - RNA, 145 nt, synthetic construct
  source 1..145: mol_type = other RNA
gctggtacacggcagggtcagttttagagctagaaatagcaagttaaaataaggctagtccgttatcaacttgaaaaagtggcaccgagtcggtgcggcttgtcgacgacggcggtctccgtcgtcaggatcatccctgccgtgt

SEQ ID NO: 403 - RNA, 146 nt, synthetic construct
  source 1..146: mol_type = other RNA
gctggtacacggcagggtcagttttagagctagaaatagcaagttaaaataaggctagtccgttatcaacttgaaaaagtggcaccgagtcggtgcggcttgtcgacgacggcggtctccgtcgtcaggatcatccctgccgtttt

SEQ ID NO: 404 - RNA, 146 nt, synthetic construct
  source 1..146: mol_type = other RNA
gctggtacacggcagggtcagttttagagctagaaatagcaagttaaaataaggctagtccgttatcaacttgaaaaagtggcaccgagtcggtgcggcttgtcgacgacggcggtctccgtcgtcaggatcatccctgccgtgta

SEQ ID NO: 405 - RNA, 147 nt, synthetic construct
  source 1..147: mol_type = other RNA
gctggtacacggcagggtcagttttagagctagaaatagcaagttaaaataaggctagtccgttatcaacttgaaaaagtggcaccgagtcggtgcggcttgtcgacgacggcggtctccgtcgtcaggatcatccctgccgttttt

SEQ ID NO: 406 - RNA, 147 nt, synthetic construct
  source 1..147: mol_type = other RNA
gctggtacacggcagggtcagttttagagctagaaatagcaagttaaaataaggctagtccgttatcaacttgaaaaagtggcaccgagtcggtgcggcttgtcgacgacggcggtctccgtcgtcaggatcatccctgccgtgtac

SEQ ID NO: 407 - RNA, 148 nt, synthetic construct
  source 1..148: mol_type = other RNA
gctggtacacggcagggtcagttttagagctagaaatagcaagttaaaataaggctagtccgttatcaacttgaaaaagtggcaccgagtcggtgcggcttgtcgacgacggcggtctccgtcgtcaggatcatccctgccgtgtttt

SEQ ID NO: 408 - RNA, 149 nt, synthetic construct
  source 1..149: mol_type = other RNA
gctggtacacggcagggtcagttttagagctagaaatagcaagttaaaataaggctagtccgttatcaacttgaaaaagtggcaccgagtcggtgcggcttgtcgacgacggcggtctccgtcgtcaggatcatccctgccgtgttttt

SEQ ID NO: 409 - RNA, 150 nt, synthetic construct
  source 1..150: mol_type = other RNA
gctggtacacggcagggtcagttttagagctagaaatagcaagttaaaataaggctagtccgttatcaacttgaaaaagtggcaccgagtcggtgcggcttgtcgacgacggcggtctccgtcgtcaggatcatccctgccgtgtatttt

SEQ ID NO: 410 - RNA, 151 nt, synthetic construct
  source 1..151: mol_type = other RNA
gctggtacacggcagggtcagttttagagctagaaatagcaagttaaaataaggctagtccgttatcaacttgaaaaagtggcaccgagtcggtgcggcttgtcgacgacggcggtctccgtcgtcaggatcatccctgccgtgtactttt

SEQ ID NO: 411 - RNA, 17 nt, synthetic construct
  source 1..17: mol_type = other RNA
acaagaggatcagggtt

SEQ ID NO: 412 - RNA, 18 nt, synthetic construct
  source 1..18: mol_type = other RNA
gacaagaggatcagggtt

SEQ ID NO: 413 - RNA, 19 nt, synthetic construct
  source 1..19: mol_type = other RNA
ggacaagaggatcagggtt

SEQ ID NO: 414 - RNA, 20 nt, synthetic construct
  source 1..20: mol_type = other RNA
gggacaagaggatcagggtt

SEQ ID NO: 415 - RNA, 21 nt, synthetic construct
  source 1..21: mol_type = other RNA
tgggacaagaggatcagggtt

SEQ ID NO: 416 - RNA, 22 nt, synthetic construct
  source 1..22: mol_type = other RNA
gtgggacaagaggatcagggtt

SEQ ID NO: 417-421 - skipped (5 entries)

SEQ ID NO: 422 - RNA, 10 nt, synthetic construct
  source 1..10: mol_type = other RNA
cctgatcctc

SEQ ID NO: 423 - RNA, 11 nt, synthetic construct
  source 1..11: mol_type = other RNA
cctgatcctct

SEQ ID NO: 424 - RNA, 12 nt, synthetic construct
  source 1..12: mol_type = other RNA
cctgatcctctt

SEQ ID NO: 425 - RNA, 13 nt, synthetic construct
  source 1..13: mol_type = other RNA
cctgatcctcttg

SEQ ID NO: 426 - RNA, 14 nt, synthetic construct
  source 1..14: mol_type = other RNA
cctgatcctcttgt

SEQ ID NO: 427 - RNA, 15 nt, synthetic construct
  source 1..15: mol_type = other RNA
cctgatcctcttgtc

SEQ ID NO: 428 - RNA, 16 nt, synthetic construct
  source 1..16: mol_type = other RNA
cctgatcctcttgtcc

SEQ ID NO: 429 - RNA, 17 nt, synthetic construct
  source 1..17: mol_type = other RNA
cctgatcctcttgtccc

SEQ ID NO: 430 - RNA, 18 nt, synthetic construct
  source 1..18: mol_type = other RNA
cctgatcctcttgtccca

SEQ ID NO: 431 - RNA, 19 nt, synthetic construct
  source 1..19: mol_type = other RNA
cctgatcctcttgtcccac

SEQ ID NO: 432 - RNA, 143 nt, synthetic construct
  source 1..143: mol_type = other RNA
gggacaagaggatcagggttgttttagagctagaaatagcaagttaaaataaggctagtccgttatcaacttgaaaaagtggcaccgagtcggtgcggcttgtcgacgacggcggtctccgtcgtcaggatcatcctgatcct

SEQ ID NO: 433 - RNA, 146 nt, synthetic construct
  source 1..146: mol_type = other RNA
gggacaagaggatcagggttgttttagagctagaaatagcaagttaaaataaggctagtccgttatcaacttgaaaaagtggcaccgagtcggtgcggcttgtcgacgacggcggtctccgtcgtcaggatcatcctgatcctctt

SEQ ID NO: 434 - RNA, 147 nt, synthetic construct
  source 1..147: mol_type = other RNA
gggacaagaggatcagggttgttttagagctagaaatagcaagttaaaataaggctagtccgttatcaacttgaaaaagtggcaccgagtcggtgcggcttgtcgacgacggcggtctccgtcgtcaggatcatcctgatccttttt

SEQ ID NO: 435 - RNA, 148 nt, synthetic construct
  source 1..148: mol_type = other RNA
gggacaagaggatcagggttgttttagagctagaaatagcaagttaaaataaggctagtccgttatcaacttgaaaaagtggcaccgagtcggtgcggcttgtcgacgacggcggtctccgtcgtcaggatcatcctgatcctcttgt

SEQ ID NO: 436 - RNA, 150 nt, synthetic construct
  source 1..150: mol_type = other RNA
gggacaagaggatcagggttgttttagagctagaaatagcaagttaaaataaggctagtccgttatcaacttgaaaaagtggcaccgagtcggtgcggcttgtcgacgacggcggtctccgtcgtcaggatcatcctgatcctctttttt

SEQ ID NO: 437 - RNA, 152 nt, synthetic construct
  source 1..152: mol_type = other RNA
gggacaagaggatcagggttgttttagagctagaaatagcaagttaaaataaggctagtccgttatcaacttgaaaaagtggcaccgagtcggtgcggcttgtcgacgacggcggtctccgtcgtcaggatcatcctgatcctcttgttttt

SEQ ID NO: 438 - RNA, 17 nt, synthetic construct
  source 1..17: mol_type = other RNA
agggttctggatatctg

SEQ ID NO: 439 - RNA, 18 nt, synthetic construct
  source 1..18: mol_type = other RNA
cagggttctggatatctg

SEQ ID NO: 440 - RNA, 19 nt, synthetic construct
  source 1..19: mol_type = other RNA
tcagggttctggatatctg

SEQ ID NO: 441 - RNA, 20 nt, synthetic construct
  source 1..20: mol_type = other RNA
gtcagggttctggatatctg

SEQ ID NO: 442 - RNA, 21 nt, synthetic construct
  source 1..21: mol_type = other RNA
ggtcagggttctggatatctg

SEQ ID NO: 443 - RNA, 22 nt, synthetic construct
  source 1..22: mol_type = other RNA
gggtcagggttctggatatctg

SEQ ID NO: 444-448 - skipped (5 entries)

SEQ ID NO: 449 - RNA, 10 nt, synthetic construct
  source 1..10: mol_type = other RNA
atatccagaa

SEQ ID NO: 450 - RNA, 11 nt, synthetic construct
  source 1..11: mol_type = other RNA
atatccagaac

SEQ ID NO: 451 - RNA, 12 nt, synthetic construct
  source 1..12: mol_type = other RNA
atatccagaacc

SEQ ID NO: 452 - RNA, 13 nt, synthetic construct
  source 1..13: mol_type = other RNA
atatccagaaccc

SEQ ID NO: 453 - RNA, 14 nt, synthetic construct
  source 1..14: mol_type = other RNA
atatccagaaccct

SEQ ID NO: 454 - RNA, 15 nt, synthetic construct
  source 1..15: mol_type = other RNA
atatccagaaccctg

SEQ ID NO: 455 - RNA, 16 nt, synthetic construct
  source 1..16: mol_type = other RNA
atatccagaaccctga

SEQ ID NO: 456 - RNA, 17 nt, synthetic construct
  source 1..17: mol_type = other RNA
atatccagaaccctgac

SEQ ID NO: 457 - RNA, 18 nt, synthetic construct
  source 1..18: mol_type = other RNA
atatccagaaccctgacc

SEQ ID NO: 458 - RNA, 19 nt, synthetic construct
  source 1..19: mol_type = other RNA
atatccagaaccctgaccc

SEQ ID NO: 459 - RNA, 144 nt, synthetic construct
  source 1..144: mol_type = other RNA
gtcagggttctggatatctggttttagagctagaaatagcaagttaaaataaggctagtccgttatcaacttgaaaaagtggcaccgagtcggtgcggcttgtcgacgacggcggtctccgtcgtcaggatcatatatccagaa

SEQ ID NO: 460 - RNA, 146 nt, synthetic construct
  source 1..146: mol_type = other RNA
gtcagggttctggatatctggttttagagctagaaatagcaagttaaaataaggctagtccgttatcaacttgaaaaagtggcaccgagtcggtgcggcttgtcgacgacggcggtctccgtcgtcaggatcatatatccagaacc

SEQ ID NO: 461 - RNA, 148 nt, synthetic construct
  source 1..148: mol_type = other RNA
gtcagggttctggatatctggttttagagctagaaatagcaagttaaaataaggctagtccgttatcaacttgaaaaagtggcaccgagtcggtgcggcttgtcgacgacggcggtctccgtcgtcaggatcatatatccagaatttt

SEQ ID NO: 462 - RNA, 148 nt, synthetic construct
  source 1..148: mol_type = other RNA
gtcagggttctggatatctggttttagagctagaaatagcaagttaaaataaggctagtccgttatcaacttgaaaaagtggcaccgagtcggtgcggcttgtcgacgacggcggtctccgtcgtcaggatcatatatccagaaccct

SEQ ID NO: 463 - RNA, 150 nt, synthetic construct
  source 1..150: mol_type = other RNA
gtcagggttctggatatctggttttagagctagaaatagcaagttaaaataaggctagtccgttatcaacttgaaaaagtggcaccgagtcggtgcggcttgtcgacgacggcggtctccgtcgtcaggatcatatatccagaacctttt

SEQ ID NO: 464 - RNA, 152 nt, synthetic construct
  source 1..152: mol_type = other RNA
gtcagggttctggatatctggttttagagctagaaatagcaagttaaaataaggctagtccgttatcaacttgaaaaagtggcaccgagtcggtgcggcttgtcgacgacggcggtctccgtcgtcaggatcatatatccagaacccttttt

SEQ ID NO: 465 - RNA, 17 nt, synthetic construct
  source 1..17: mol_type = other RNA
gcagacagacttgtcac

SEQ ID NO: 466 - RNA, 18 nt, synthetic construct
  source 1..18: mol_type = other RNA
ggcagacagacttgtcac

SEQ ID NO: 467 - RNA, 19 nt, synthetic construct
  source 1..19: mol_type = other RNA
aggcagacagacttgtcac

SEQ ID NO: 468 - RNA, 20 nt, synthetic construct
  source 1..20: mol_type = other RNA
taggcagacagacttgtcac

SEQ ID NO: 469 - RNA, 21 nt, synthetic construct
  source 1..21: mol_type = other RNA
ataggcagacagacttgtcac

SEQ ID NO: 470 - RNA, 22 nt, synthetic construct
  source 1..22: mol_type = other RNA
aataggcagacagacttgtcac

SEQ ID NO: 471-475 - skipped (5 entries)

SEQ ID NO: 476 - RNA, 10 nt, synthetic construct
  source 1..10: mol_type = other RNA
acaagtctgt

SEQ ID NO: 477 - RNA, 11 nt, synthetic construct
  source 1..11: mol_type = other RNA
acaagtctgtc

SEQ ID NO: 478 - RNA, 12 nt, synthetic construct
  source 1..12: mol_type = other RNA
acaagtctgtct

SEQ ID NO: 479 - RNA, 13 nt, synthetic construct
  source 1..13: mol_type = other RNA
acaagtctgtctg

SEQ ID NO: 480 - RNA, 14 nt, synthetic construct
  source 1..14: mol_type = other RNA
acaagtctgtctgc

SEQ ID NO: 481 - RNA, 15 nt, synthetic construct
  source 1..15: mol_type = other RNA
acaagtctgtctgcc

SEQ ID NO: 482 - RNA, 16 nt, synthetic construct
  source 1..16: mol_type = other RNA
acaagtctgtctgcct

SEQ ID NO: 483 - RNA, 17 nt, synthetic construct
  source 1..17: mol_type = other RNA
acaagtctgtctgccta

SEQ ID NO: 484 - RNA, 18 nt, synthetic construct
  source 1..18: mol_type = other RNA
acaagtctgtctgcctat

SEQ ID NO: 485 - RNA, 19 nt, synthetic construct
  source 1..19: mol_type = other RNA
acaagtctgtctgcctatt

SEQ ID NO: 486 - RNA, 144 nt, synthetic construct
  source 1..144: mol_type = other RNA
taggcagacagacttgtcacgttttagagctagaaatagcaagttaaaataaggctagtccgttatcaacttgaaaaagtggcaccgagtcggtgcggcttgtcgacgacggcggtctccgtcgtcaggatcatacaagtctgt

SEQ ID NO: 487 - RNA, 146 nt, synthetic construct
  source 1..146: mol_type = other RNA
taggcagacagacttgtcacgttttagagctagaaatagcaagttaaaataaggctagtccgttatcaacttgaaaaagtggcaccgagtcggtgcggcttgtcgacgacggcggtctccgtcgtcaggatcatacaagtctgtct

SEQ ID NO: 488 - RNA, 148 nt, synthetic construct
  source 1..148: mol_type = other RNA
taggcagacagacttgtcacgttttagagctagaaatagcaagttaaaataaggctagtccgttatcaacttgaaaaagtggcaccgagtcggtgcggcttgtcgacgacggcggtctccgtcgtcaggatcatacaagtctgttttt

SEQ ID NO: 489 - RNA, 148 nt, synthetic construct
  source 1..148: mol_type = other RNA
taggcagacagacttgtcacgttttagagctagaaatagcaagttaaaataaggctagtccgttatcaacttgaaaaagtggcaccgagtcggtgcggcttgtcgacgacggcggtctccgtcgtcaggatcatacaagtctgtctgc

SEQ ID NO: 490 - RNA, 150 nt, synthetic construct
  source 1..150: mol_type = other RNA
taggcagacagacttgtcacgttttagagctagaaatagcaagttaaaataaggctagtccgttatcaacttgaaaaagtggcaccgagtcggtgcggcttgtcgacgacggcggtctccgtcgtcaggatcatacaagtctgtcttttt

SEQ ID NO: 491 - RNA, 152 nt, synthetic construct
  source 1..152: mol_type = other RNA
taggcagacagacttgtcacgttttagagctagaaatagcaagttaaaataaggctagtccgttatcaacttgaaaaagtggcaccgagtcggtgcggcttgtcgacgacggcggtctccgtcgtcaggatcatacaagtctgtctgctttt

SEQ ID NO: 492 - RNA, 17 nt, synthetic construct
  source 1..17: mol_type = other RNA
gggttctggatatctgt

SEQ ID NO: 493 - RNA, 18 nt, synthetic construct
  source 1..18: mol_type = other RNA
agggttctggatatctgt

SEQ ID NO: 494 - RNA, 19 nt, synthetic construct
  source 1..19: mol_type = other RNA
cagggttctggatatctgt

SEQ ID NO: 495 - RNA, 20 nt, synthetic construct
  source 1..20: mol_type = other RNA
tcagggttctggatatctgt

SEQ ID NO: 496 - RNA, 21 nt, synthetic construct
  source 1..21: mol_type = other RNA
gtcagggttctggatatctgt

SEQ ID NO: 497 - RNA, 22 nt, synthetic construct
  source 1..22: mol_type = other RNA
ggtcagggttctggatatctgt

SEQ ID NO: 498-502 - skipped (5 entries)

SEQ ID NO: 503 - RNA, 10 nt, synthetic construct
  source 1..10: mol_type = other RNA
gatatccaga

SEQ ID NO: 504 - RNA, 11 nt, synthetic construct
  source 1..11: mol_type = other RNA
gatatccagaa

SEQ ID NO: 505 - RNA, 12 nt, synthetic construct
  source 1..12: mol_type = other RNA
gatatccagaac

SEQ ID NO: 506 - RNA, 13 nt, synthetic construct
  source 1..13: mol_type = other RNA
gatatccagaacc

SEQ ID NO: 507 - RNA, 14 nt, synthetic construct
  source 1..14: mol_type = other RNA
gatatccagaaccc

SEQ ID NO: 508 - RNA, 15 nt, synthetic construct
  source 1..15: mol_type = other RNA
gatatccagaaccct

SEQ ID NO: 509 - RNA, 16 nt, synthetic construct
  source 1..16: mol_type = other RNA
gatatccagaaccctg

SEQ ID NO: 510 - RNA, 17 nt, synthetic construct
  source 1..17: mol_type = other RNA
gatatccagaaccctga

SEQ ID NO: 511 - RNA, 18 nt, synthetic construct
  source 1..18: mol_type = other RNA
gatatccagaaccctgac

SEQ ID NO: 512 - RNA, 19 nt, synthetic construct
  source 1..19: mol_type = other RNA
gatatccagaaccctgacc

SEQ ID NO: 513 - RNA, 144 nt, synthetic construct
  source 1..144: mol_type = other RNA
tcagggttctggatatctgtgttttagagctagaaatagcaagttaaaataaggctagtccgttatcaacttgaaaaagtggcaccgagtcggtgcggcttgtcgacgacggcggtctccgtcgtcaggatcatgatatccaga

SEQ ID NO: 514 - RNA, 146 nt, synthetic construct
  source 1..146: mol_type = other RNA
tcagggttctggatatctgtgttttagagctagaaatagcaagttaaaataaggctagtccgttatcaacttgaaaaagtggcaccgagtcggtgcggcttgtcgacgacggcggtctccgtcgtcaggatcatgatatccagaac

SEQ ID NO: 515 - RNA, 148 nt, synthetic construct
  source 1..148: mol_type = other RNA
tcagggttctggatatctgtgttttagagctagaaatagcaagttaaaataaggctagtccgttatcaacttgaaaaagtggcaccgagtcggtgcggcttgtcgacgacggcggtctccgtcgtcaggatcatgatatccagatttt

SEQ ID NO: 516 - RNA, 149 nt, synthetic construct
  source 1..149: mol_type = other RNA
tcagggttctggatatctgtgttttagagctagaaatagcaagttaaaataaggctagtccgttatcaacttgaaaaagtggcaccgagtcggtgcggcttgtcgacgacggcggtctccgtcgtcaggatcatgatatccagaaccct

SEQ ID NO: 517 - RNA, 150 nt, synthetic construct
  source 1..150: mol_type = other RNA
tcagggttctggatatctgtgttttagagctagaaatagcaagttaaaataaggctagtccgttatcaacttgaaaaagtggcaccgagtcggtgcggcttgtcgacgacggcggtctccgtcgtcaggatcatgatatccagaactttt

SEQ ID NO: 518 - RNA, 153 nt, synthetic construct
  source 1..153: mol_type = other RNA
tcagggttctggatatctgtgttttagagctagaaatagcaagttaaaataaggctagtccgttatcaacttgaaaaagtggcaccgagtcggtgcggcttgtcgacgacggcggtctccgtcgtcaggatcatgatatccagaacccttttt

SEQ ID NO: 519 - RNA, 17 nt, synthetic construct
  source 1..17: mol_type = other RNA
ctcagctggtacacggc

SEQ ID NO: 520 - RNA, 18 nt, synthetic construct
  source 1..18: mol_type = other RNA
tctcagctggtacacggc

SEQ ID NO: 521 - RNA, 19 nt, synthetic construct
  source 1..19: mol_type = other RNA
ctctcagctggtacacggc

SEQ ID NO: 522 - RNA, 20 nt, synthetic construct
  source 1..20: mol_type = other RNA
tctctcagctggtacacggc

SEQ ID NO: 523 - RNA, 21 nt, synthetic construct
  source 1..21: mol_type = other RNA
gtctctcagctggtacacggc

SEQ ID NO: 524 - RNA, 22 nt, synthetic construct
  source 1..22: mol_type = other RNA
agtctctcagctggtacacggc

SEQ ID NO: 525-529 - skipped (5 entries)

SEQ ID NO: 530 - RNA, 10 nt, synthetic construct
  source 1..10: mol_type = other RNA
gtgtaccagc

SEQ ID NO: 531 - RNA, 11 nt, synthetic construct
  source 1..11: mol_type = other RNA
gtgtaccagct

SEQ ID NO: 532 - RNA, 12 nt, synthetic construct
  source 1..12: mol_type = other RNA
gtgtaccagctg

SEQ ID NO: 533 - RNA, 13 nt, synthetic construct
  source 1..13: mol_type = other RNA
gtgtaccagctga

SEQ ID NO: 534 - RNA, 14 nt, synthetic construct
  source 1..14: mol_type = other RNA
gtgtaccagctgag

SEQ ID NO: 535 - RNA, 15 nt, synthetic construct
  source 1..15: mol_type = other RNA
gtgtaccagctgaga

SEQ ID NO: 536 - RNA, 16 nt, synthetic construct
  source 1..16: mol_type = other RNA
gtgtaccagctgagag

SEQ ID NO: 537 - RNA, 17 nt, synthetic construct
  source 1..17: mol_type = other RNA
gtgtaccagctgagaga

SEQ ID NO: 538 - RNA, 18 nt, synthetic construct
  source 1..18: mol_type = other RNA
gtgtaccagctgagagac

SEQ ID NO: 539 - RNA, 19 nt, synthetic construct
  source 1..19: mol_type = other RNA
gtgtaccagctgagagact

SEQ ID NO: 540 - RNA, 137 nt, synthetic construct
  source 1..137: mol_type = other RNA
tctctcagctggtacacggcgttttagagctagaaatagcaagttaaaataaggctagtccgttatcaacttgaaaaagtggcaccgagtcggtgcgacgacggcggtctccgtcgtcaggatcatgtgtaccagct

SEQ ID NO: 541 - RNA, 139 nt, synthetic construct
  source 1..139: mol_type = other RNA
tctctcagctggtacacggcgttttagagctagaaatagcaagttaaaataaggctagtccgttatcaacttgaaaaagtggcaccgagtcggtgcgacgacggcggtctccgtcgtcaggatcatgtgtaccagctga

SEQ ID NO: 542 - RNA, 139 nt, synthetic construct
  source 1..139: mol_type = other RNA
tctctcagctggtacacggcgttttagagctagaaatagcaagttaaaataaggctagtccgttatcaacttgaaaaagtggcaccgagtcggtgcgacgacggagaccgccgtcgtcgacaagccgtgtaccagctga

SEQ ID NO: 543 - RNA, 141 nt, synthetic construct
  source 1..141: mol_type = other RNA
tctctcagctggtacacggcgttttagagctagaaatagcaagttaaaataaggctagtccgttatcaacttgaaaaagtggcaccgagtcggtgcgacgacggcggtctccgtcgtcaggatcatgtgtaccagcttttt

SEQ ID NO: 544 - RNA, 141 nt, synthetic construct
  source 1..141: mol_type = other RNA
tctctcagctggtacacggcgttttagagctagaaatagcaagttaaaataaggctagtccgttatcaacttgaaaaagtggcaccgagtcggtgcgacgacggcggtctccgtcgtcaggatcatgtgtaccagctgaga

SEQ ID NO: 545 - RNA, 143 nt, synthetic construct
  source 1..143: mol_type = other RNA
tctctcagctggtacacggcgttttagagctagaaatagcaagttaaaataaggctagtccgttatcaacttgaaaaagtggcaccgagtcggtgcgacgacggcggtctccgtcgtcaggatcatgtgtaccagctgatttt

SEQ ID NO: 546 - RNA, 143 nt, synthetic construct
  source 1..143: mol_type = other RNA
tctctcagctggtacacggcgttttagagctagaaatagcaagttaaaataaggctagtccgttatcaacttgaaaaagtggcaccgagtcggtgcgacgacggagaccgccgtcgtcgacaagccgtgtaccagctgatttt

SEQ ID NO: 547 - RNA, 143 nt, synthetic construct
  source 1..143: mol_type = other RNA
tctctcagctggtacacggcgttttagagctagaaatagcaagttaaaataaggctagtccgttatcaacttgaaaaagtggcaccgagtcggtgcggcttgtcgacgacggcggtctccgtcgtcaggatcatgtgtaccag

SEQ ID NO: 548 - RNA, 143 nt, synthetic construct
  source 1..143: mol_type = other RNA
tctctcagctggtacacggcgttttagagctagaaatagcaagttaaaataaggctagtccgttatcaacttgaaaaagtggcaccgagtcggtgcgacgacggcggtctccgtcgtcaggatcatgtgtaccagctgagaga

SEQ ID NO: 549 - RNA, 145 nt, synthetic construct
  source 1..145: mol_type = other RNA
tctctcagctggtacacggcgttttagagctagaaatagcaagttaaaataaggctagtccgttatcaacttgaaaaagtggcaccgagtcggtgcgacgacggcggtctccgtcgtcaggatcatgtgtaccagctgagatttt

SEQ ID NO: 550 - RNA, 145 nt, synthetic construct
  source 1..145: mol_type = other RNA
tctctcagctggtacacggcgttttagagctagaaatagcaagttaaaataaggctagtccgttatcaacttgaaaaagtggcaccgagtcggtgcggcttgtcgacgacggcggtctccgtcgtcaggatcatgtgtaccagct

SEQ ID NO: 551 - RNA, 147 nt, synthetic construct
  source 1..147: mol_type = other RNA
tctctcagctggtacacggcgttttagagctagaaatagcaagttaaaataaggctagtccgttatcaacttgaaaaagtggcaccgagtcggtgcggcttgtcgacgacggcggtctccgtcgtcaggatcatgtgtaccagtttt

SEQ ID NO: 552 - RNA, 147 nt, synthetic construct
  source 1..147: mol_type = other RNA
tctctcagctggtacacggcgttttagagctagaaatagcaagttaaaataaggctagtccgttatcaacttgaaaaagtggcaccgagtcggtgcgacgacggcggtctccgtcgtcaggatcatgtgtaccagctgagagatttt

SEQ ID NO: 553 - RNA, 147 nt, synthetic construct
  source 1..147: mol_type = other RNA
tctctcagctggtacacggcgttttagagctagaaatagcaagttaaaataaggctagtccgttatcaacttgaaaaagtggcaccgagtcggtgcgacgacggcggtctccgtcgtcaggatcatgtgtaccagctgatttttttt

SEQ ID NO: 554 - RNA, 147 nt, synthetic construct
  source 1..147: mol_type = other RNA
tctctcagctggtacacggcgttttagagctagaaatagcaagttaaaataaggctagtccgttatcaacttgaaaaagtggcaccgagtcggtgcggcttgtcgacgacggcggtctccgtcgtcaggatcatgtgtaccagctga

SEQ ID NO: 555 - RNA, 147 nt, synthetic construct
  source 1..147: mol_type = other RNA
tctctcagctggtacacggcgttttagagctagaaatagcaagttaaaataaggctagtccgttatcaacttgaaaaagtggcaccgagtcggtgcatgatcctgacgacggagaccgccgtcgtcgacaagccgtgtaccagctga

SEQ ID NO: 556 - RNA, 149 nt, synthetic construct
  source 1..149: mol_type = other RNA
tctctcagctggtacacggcgttttagagctagaaatagcaagttaaaataaggctagtccgttatcaacttgaaaaagtggcaccgagtcggtgcggcttgtcgacgacggcggtctccgtcgtcaggatcatgtgtaccagcttttt

SEQ ID NO: 557 - RNA, 149 nt, synthetic construct
  source 1..149: mol_type = other RNA
tctctcagctggtacacggcgttttagagctagaaatagcaagttaaaataaggctagtccgttatcaacttgaaaaagtggcaccgagtcggtgcggcttgtcgacgacggcggtctccgtcgtcaggatcatgtgtaccagctgaga

SEQ ID NO: 558 - RNA, 151 nt, synthetic construct
  source 1..151: mol_type = other RNA
tctctcagctggtacacggcgttttagagctagaaatagcaagttaaaataaggctagtccgttatcaacttgaaaaagtggcaccgagtcggtgcggcttgtcgacgacggcggtctccgtcgtcaggatcatgtgtaccagctgatttt

SEQ ID NO: 559 - RNA, 151 nt, synthetic construct
  source 1..151: mol_type = other RNA
tctctcagctggtacacggcgttttagagctagaaatagcaagttaaaataaggctagtccgttatcaacttgaaaaagtggcaccgagtcggtgcatgatcctgacgacggagaccgccgtcgtcgacaagccgtgtaccagctgatttt

SEQ ID NO: 560 - RNA, 151 nt, synthetic construct
  source 1..151: mol_type = other RNA
tctctcagctggtacacggcgttttagagctagaaatagcaagttaaaataaggctagtccgttatcaacttgaaaaagtggcaccgagtcggtgcggcttgtcgacgacggcggtctccgtcgtcaggatcatgtgtaccagctgagaga

SEQ ID NO: 561 - RNA, 153 nt, synthetic construct
  source 1..153: mol_type = other RNA
tctctcagctggtacacggcgttttagagctagaaatagcaagttaaaataaggctagtccgttatcaacttgaaaaagtggcaccgagtcggtgcggcttgtcgacgacggcggtctccgtcgtcaggatcatgtgtaccagctgagatttt

SEQ ID NO: 562 - RNA, 155 nt, synthetic construct
  source 1..155: mol_type = other RNA
tctctcagctggtacacggcgttttagagctagaaatagcaagttaaaataaggctagtccgttatcaacttgaaaaagtggcaccgagtcggtgcggcttgtcgacgacggcggtctccgtcgtcaggatcatgtgtaccagctgagagatttt

SEQ ID NO: 563 - RNA, 17 nt, synthetic construct
  source 1..17: mol_type = other RNA
gtgggacaagaggatca

SEQ ID NO: 564 - RNA, 18 nt, synthetic construct
  source 1..18: mol_type = other RNA
tgtgggacaagaggatca

SEQ ID NO: 565 - RNA, 19 nt, synthetic construct
  source 1..19: mol_type = other RNA
ctgtgggacaagaggatca

SEQ ID NO: 566 - RNA, 20 nt, synthetic construct
  source 1..20: mol_type = other RNA
tctgtgggacaagaggatca

SEQ ID NO: 567 - RNA, 21 nt, synthetic construct
  source 1..21: mol_type = other RNA
atctgtgggacaagaggatca

SEQ ID NO: 568 - RNA, 22 nt, synthetic construct
  source 1..22: mol_type = other RNA
tatctgtgggacaagaggatca

SEQ ID NO: 569-573 - skipped (5 entries)

SEQ ID NO: 574 - RNA, 10 nt, synthetic construct
  source 1..10: mol_type = other RNA
tcctcttgtc

SEQ ID NO: 575 - RNA, 11 nt, synthetic construct
  source 1..11: mol_type = other RNA
tcctcttgtcc

SEQ ID NO: 576 - RNA, 12 nt, synthetic construct
  source 1..12: mol_type = other RNA
tcctcttgtccc

SEQ ID NO: 577 - RNA, 13 nt, synthetic construct
  source 1..13: mol_type = other RNA
tcctcttgtccca

SEQ ID NO: 578 - RNA, 14 nt, synthetic construct
  source 1..14: mol_type = other RNA
tcctcttgtcccac

SEQ ID NO: 579 - RNA, 15 nt, synthetic construct
  source 1..15: mol_type = other RNA
tcctcttgtcccaca

SEQ ID NO: 580 - RNA, 16 nt, synthetic construct
  source 1..16: mol_type = other RNA
tcctcttgtcccacag

SEQ ID NO: 581 - RNA, 17 nt, synthetic construct
  source 1..17: mol_type = other RNA
tcctcttgtcccacaga

SEQ ID NO: 582 - RNA, 18 nt, synthetic construct
  source 1..18: mol_type = other RNA
tcctcttgtcccacagat

SEQ ID NO: 583 - RNA, 19 nt, synthetic construct
  source 1..19: mol_type = other RNA
tcctcttgtcccacagata

SEQ ID NO: 584 - RNA, 143 nt, synthetic construct
  source 1..143: mol_type = other RNA
tctgtgggacaagaggatcagttttagagctagaaatagcaagttaaaataaggctagtccgttatcaacttgaaaaagtggcaccgagtcggtgcggcttgtcgacgacggcggtctccgtcgtcaggatcattcctcttgt

SEQ ID NO: 585 - RNA, 145 nt, synthetic construct
  source 1..145: mol_type = other RNA
tctgtgggacaagaggatcagttttagagctagaaatagcaagttaaaataaggctagtccgttatcaacttgaaaaagtggcaccgagtcggtgcggcttgtcgacgacggcggtctccgtcgtcaggatcattcctcttgtcc

SEQ ID NO: 586 - RNA, 147 nt, synthetic construct
  source 1..147: mol_type = other RNA
tctgtgggacaagaggatcagttttagagctagaaatagcaagttaaaataaggctagtccgttatcaacttgaaaaagtggcaccgagtcggtgcggcttgtcgacgacggcggtctccgtcgtcaggatcattcctcttgttttt

SEQ ID NO: 587 - RNA, 147 nt, synthetic construct
  source 1..147: mol_type = other RNA
tctgtgggacaagaggatcagttttagagctagaaatagcaagttaaaataaggctagtccgttatcaacttgaaaaagtggcaccgagtcggtgcggcttgtcgacgacggcggtctccgtcgtcaggatcattcctcttgtccca

SEQ ID NO: 588 - RNA, 149 nt, synthetic construct
  source 1..149: mol_type = other RNA
tctgtgggacaagaggatcagttttagagctagaaatagcaagttaaaataaggctagtccgttatcaacttgaaaaagtggcaccgagtcggtgcggcttgtcgacgacggcggtctccgtcgtcaggatcattcctcttgtcctttt

SEQ ID NO: 589 - RNA, 151 nt, synthetic construct
  source 1..151: mol_type = other RNA
tctgtgggacaagaggatcagttttagagctagaaatagcaagttaaaataaggctagtccgttatcaacttgaaaaagtggcaccgagtcggtgcggcttgtcgacgacggcggtctccgtcgtcaggatcattcctcttgtcccatttt

SEQ ID NO: 590 - RNA, 76 nt, synthetic construct
  source 1..76: mol_type = other RNA
gttttagagctagaaatagcaagttaaaataaggctagtccgttatcaacttgaaaaagtggcaccgagtcggtgc

SEQ ID NO: 591 - RNA, 147 nt, synthetic construct
  source 1..147: mol_type = other RNA
agagtctctcagctggtacagttttagagctagaaatagcaagttaaaataaggctagtccgttatcaacttgaaaaagtggcaccgagtcggtgcatgatcctgacgacggagaccgccgtcgtcgacaagccaccagctgagaga

SEQ ID NO: 592 - RNA, 140 nt, synthetic construct
  source 1..140: mol_type = other RNA
gctggactccagcctgggttggttttagagctagaaatagcaagttaaaataaggctagtccgttatcaacttgaaaaagtggcaccgagtcggtgcgcatacgcccttcgagcatgtaggtcacggcccaggctggagt

SEQ ID NO: 593 - RNA, 149 nt, synthetic construct
  source 1..149: mol_type = other RNA
gctggactccagcctgggttggttttagagctagaaatagcaagttaaaataaggctagtccgttatcaacttgaaaaagtggcaccgagtcggtgccttcgtggcgcatacgcccttcgagcatgtaggtcacggcccaggctggagt

SEQ ID NO: 594 - RNA, 137 nt, synthetic construct
  source 1..137: mol_type = other RNA
gctggactccagcctgggttggttttagagctagaaatagcaagttaaaataaggctagtccgttatcaacttgaaaaagtggcaccgagtcggtgctacgcccttcgagcatgtaggtcacggcccaggctggagt

SEQ ID NO: 595 - RNA, 139 nt, synthetic construct
  source 1..139: mol_type = other RNA
agagtctctcagctggtacagttttagagctagaaatagcaagttaaaataaggctagtccgttatcaacttgaaaaagtggcaccgagtcggtgcacatgctcgaagggcgtatgcgccacgaagaccagctgagaga

SEQ ID NO: 596 - RNA, 143 nt, synthetic construct
  source 1..143: mol_type = other RNA
agagtctctcagctggtacagttttagagctagaaatagcaagttaaaataaggctagtccgttatcaacttgaaaaagtggcaccgagtcggtgcacatgctcgaagggcgtatgcgccacgaagaccagctgagagatttt

SEQ ID NO: 597 - RNA, 148 nt, synthetic construct
  source 1..148: mol_type = other RNA
agagtctctcagctggtacagttttagagctagaaatagcaagttaaaataaggctagtccgttatcaacttgaaaaagtggcaccgagtcggtgcccgtgacctacatgctcgaagggcgtatgcgccacgaagaccagctgagaga

SEQ ID NO: 598 - RNA, 152 nt, synthetic construct
  source 1..152: mol_type = other RNA
agagtctctcagctggtacagttttagagctagaaatagcaagttaaaataaggctagtccgttatcaacttgaaaaagtggcaccgagtcggtgcccgtgacctacatgctcgaagggcgtatgcgccacgaagaccagctgagagatttt

SEQ ID NO: 599 - RNA, 136 nt, synthetic construct
  source 1..136: mol_type = other RNA
agagtctctcagctggtacagttttagagctagaaatagcaagttaaaataaggctagtccgttatcaacttgaaaaagtggcaccgagtcggtgccctacatgctcgaagggcgtatgcgccaccagctgagaga

SEQ ID NO: 600 - RNA, 140 nt, synthetic construct
  source 1..140: mol_type = other RNA
agagtctctcagctggtacagttttagagctagaaatagcaagttaaaataaggctagtccgttatcaacttgaaaaagtggcaccgagtcggtgccctacatgctcgaagggcgtatgcgccaccagctgagagatttt

SEQ ID NO: 601 - RNA, 142 nt, synthetic construct
  source 1..142: mol_type = other RNA
agagtctctcagctggtacagttttagagctagaaatagcaagttaaaataaggctagtccgttatcaacttgaaaaagtggcaccgagtcggtgcccgtgacctacatgctcgaagggcgtatgcgccaccagctgagaga

SEQ ID NO: 602 - RNA, 146 nt, synthetic construct
  source 1..146: mol_type = other RNA
agagtctctcagctggtacagttttagagctagaaatagcaagttaaaataaggctagtccgttatcaacttgaaaaagtggcaccgagtcggtgcccgtgacctacatgctcgaagggcgtatgcgccaccagctgagagatttt

SEQ ID NO: 603 - skipped

SEQ ID NO: 604 - RNA, 76 nt, synthetic construct
  source 1..76: mol_type = other RNA
gtttaagagctagaaatagcaagtttaaataaggctagtccgttatcagcgtgaaaacgcggcaccgagtcggtgc

SEQ ID NO: 605 - RNA, 86 nt, synthetic construct
  source 1..86: mol_type = other RNA
gtttaagagctatgctggaaacagcatagcaagtttaaataaggctagtccgttatcaacttgaaaaagtggcaccgagtcggtgc

SEQ ID NO: 606 - RNA, 76 nt, synthetic construct
  source 1..76: mol_type = other RNA
gtttgagagctagaaatagcaagtttaaataaggctagtccgttatcaacttgaaaaagtgggaccgagtcggtcc

SEQ ID NO: 607 - RNA, 76 nt, synthetic construct
  source 1..76: mol_type = other RNA
gtttaagagctagaaatagcaagtttaaataaggctagtccgttatcaacttgaaaaagtggcaccgagtcggtgc

SEQ ID NO: 608 - RNA, 88 nt, synthetic construct
  source 1..88: mol_type = other RNA
gttttagagctagctcatgaaaatgagctagcaagttaaaataaggctagtccgttatcaacttgaaaaagtgggaccgagtcggtcc

SEQ ID NO: 609 - RNA, 76 nt, synthetic construct
  source 1..76: mol_type = other RNA
gttttagagctatacgtagcaagttaaaataaggctagtccgttatcaactttacgaagtggcaccgagtcggtgc

SEQ ID NO: 610 - RNA, 76 nt, synthetic construct
  source 1..76: mol_type = other RNA
gttttagagctatacgtagcaagttaaaataaggctagtccgttatcaactttacgaagtgggaccgagtcggtcc

SEQ ID NO: 611 - RNA, 86 nt, synthetic construct
  source 1..86: mol_type = other RNA
gttttagagctatgctggaaacagcatagcaagttaaaataaggctagtccgttatcaacttgaaaaagtggcaccgagtcggtgc

SEQ ID NO: 612 - RNA, 55 nt, synthetic construct
  source 1..55: mol_type = other RNA
gggtcaggagccccccccctgaacccaggataaccctcaaagtcggggggcaacc

SEQ ID NO: 613 - RNA, 60 nt, synthetic construct
  source 1..60: mol_type = other RNA
gtcagggtcaggagccccccccctgaacccaggataaccctcaaagtcggggggcaaccc

SEQ ID NO: 614 - RNA, 56 nt, synthetic construct
  source 1..56: mol_type = other RNA
gggtcaggagccccccccctgaacccaggaaaaccctcaaagtcggggggcaaccc

SEQ ID NO: 615 - RNA, 56 nt, synthetic construct
  source 1..56: mol_type = other RNA
gggtcaggagccccccccctgcacccaggaaaaccctcaaagtcggggggcaaccc

SEQ ID NO: 616 - RNA, 56 nt, synthetic construct
  source 1..56: mol_type = other RNA
gggtcaggagccccccccctgcacccaggataaccctcaaagtcggggggcaaccc

SEQ ID NO: 617 - RNA, 60 nt, synthetic construct
  source 1..60: mol_type = other RNA
gtcagggtcaggagccccccccctgaacccaggaaaaccctcaaagtcggggggcaaccc

SEQ ID NO: 618 - RNA, 60 nt, synthetic construct
  source 1..60: mol_type = other RNA
gtcagggtcaggagccccccccctgcacccaggaaaaccctcaaagtcggggggcaaccc

SEQ ID NO: 619 - RNA, 51 nt, synthetic construct
  source 1..51: mol_type = other RNA
gggtcaggagccccccccctgaacccaggataaccctcaaagtcggggggc

SEQ ID NO: 620 - RNA, 13 nt, synthetic construct
  source 1..13: mol_type = other RNA
tggtggtggtggt

SEQ ID NO: 621 - RNA, 20 nt, synthetic construct
  source 1..20: mol_type = other RNA
gggacagggcagggacaggg

SEQ ID NO: 622 - RNA, 21 nt, synthetic construct
  source 1..21: mol_type = other RNA
gggtccgggtctgggtctggg

SEQ ID NO: 623 - RNA, 19 nt, synthetic construct
  source 1..19: mol_type = other RNA
gggcagggtctgggctggg

SEQ ID NO: 624 - RNA, 19 nt, synthetic construct
  source 1..19: mol_type = other RNA
gggctgggatgggaaaggg

SEQ ID NO: 625 - RNA, 19 nt, synthetic construct
  source 1..19: mol_type = other RNA
gggctctgggtgggccggg

SEQ ID NO: 626 - RNA, 18 nt, synthetic construct
  source 1..18: mol_type = other RNA
gggctgggctgggcaggg

SEQ ID NO: 627 - RNA, 17 nt, synthetic construct
  source 1..17: mol_type = other RNA
gggtgggctgggaaggg

SEQ ID NO: 628 - RNA, 17 nt, synthetic construct
  source 1..17: mol_type = other RNA
gggagggagggctaggg

SEQ ID NO: 629 - RNA, 17 nt, synthetic construct
  source 1..17: mol_type = other RNA
gggcagggctgggaggg

SEQ ID NO: 630 - RNA, 16 nt, synthetic construct
  source 1..16: mol_type = other RNA
gggtgggagggctggg

SEQ ID NO: 631 - RNA, 43 nt, synthetic construct
  source 1..43: mol_type = other RNA
gcgtaacctccatccgagttgcaagagagggaaacgcagtctc

SEQ ID NO: 632 - RNA, 160 nt, synthetic construct
  source 1..160: mol_type = other RNA
ggaattgcgggaaaggggtcaacagccgttcagtaccaagtctcaggggaaactttgagatggccttgcaaagggtatggtaataagctgacggacatggtcctaaccacgcagccaagtcctaagtcaacagatcttctgttgatatggatgcagttca

SEQ ID NO: 633 - RNA, 42 nt, synthetic construct
  source 1..42: mol_type = other RNA
ttgacgcggttctatctagttacgcgttaaaccaactagaaa

SEQ ID NO: 634 - RNA, 42 nt, synthetic construct
  source 1..42: mol_type = other RNA
ttgacgcggttctatctacttacgcgttaaaccaactagaaa

SEQ ID NO: 635 - RNA, 44 nt, synthetic construct
  source 1..44: mol_type = other RNA
cgcgagtctaggggataacgcgttaaacttcctagaaggcggtt

SEQ ID NO: 636 - RNA, 44 nt, synthetic construct
  source 1..44: mol_type = other RNA
cgcggatctagattgtaacgcgttaaaccatctagaaggcggtt

SEQ ID NO: 637 - RNA, 44 nt, synthetic construct
  source 1..44: mol_type = other RNA
cgcgtcgctaccgcccggcgcgttaaacacactagaaggcggtt

SEQ ID NO: 638 - RNA, 37 nt, synthetic construct
  source 1..37: mol_type = other RNA
cgcggttctatctagttacgcgttaaaccaactagaa

SEQ ID NO: 639 - RNA, 68 nt, synthetic construct
  source 1..68: mol_type = other RNA
ggccggcatggtcccagcctcctcgctggcgccggctgggcaacatgcttcggcatggcgaatgggac

SEQ ID NO: 640 - RNA, 18 nt, synthetic construct
  source 1..18: mol_type = other RNA
ggtgggagacgtcccacc

SEQ ID NO: 641 - RNA, 16 nt, synthetic construct
  source 1..16: mol_type = other RNA
tgggagacgtcccacc

SEQ ID NO: 642 - RNA, 24 nt, synthetic construct
  source 1..24: mol_type = other RNA
gagcagcatggcgtcgctgctcac

SEQ ID NO: 643 - RNA, 21 nt, synthetic construct
  source 1..21: mol_type = other RNA
ccatcagttgacaccctgagg

SEQ ID NO: 644 - RNA, 28 nt, synthetic construct
  source 1..28: mol_type = other RNA
gcagacctaagtggtgacatatggtctg

SEQ ID NO: 645 - RNA, 24 nt, synthetic construct
  source 1..24: mol_type = other RNA
catgcgattagaaataatcgcatg

SEQ ID NO: 646 - RNA, 118 nt, synthetic construct
  source 1..118: mol_type = other RNA
tctgccatcaaagctgcgaccgtgctcagtctggtgggagacgtcccaccggccggcatggtcccagcctcctcgctggcgccggctgggcaacatgcttcggcatggcgaatgggac

SEQ ID NO: 647-1000 - skipped (354 entries)

SEQ ID NO: 1001 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 1002 - protein, 677 aa, Moloney murine leukemia virus
TLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 1003 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 1004 - protein, 503 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDNSRLIN

SEQ ID NO: 1005 - protein, 1368 aa, Streptococcus pyogenes [sequence omitted]

SEQ ID NO: 1006 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 1007 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 1008 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 1009 - protein, 1054 aa, Staphylococcus lugdunensis
MNQKFILGLDIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGKGSPYGWEGDPKAWYETLMGHCTYFPDELRSVKYAYSADLFNALNDLNNLVIQRDGLSKLEYHEKYHIIENVFKQKKKPTLKQIANEINVNPEDIKGYRITKSGKPQFTEFKLYHDLKSVLFDQSILENEDVLDQIAEILTIYQDKDSIKSKLTELDILLNEEDKENIAQLTGYTGTHRLSLKCIRLVLEEQWYSSRNQMEIFTHLNIKPKKINLTAANKIPKAMIDEFILSPVVKRTFGQAINLINKIIEKYGVPEDIIIELARENNSKDKQKFINEMQKKNENTRKRINEIIGKYGNQNAKRLVEKIRLHDEQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIESKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 1010 - protein, 1054 aa, synthetic construct
MNQKFILGLDIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGKGSPYGWEGDPKAWYETLMGHCTYFPDELRSVKYAYSADLFNALNDLNNLVIQRDGLSKLEYHEKYHIIENVFKQKKKPTLKQIANEINVNPEDIKGYRITKSGKPQFTEFKLYHDLKSVLFDQSILENEDVLDQIAEILTIYQDKDSIKSKLTELDILLNEEDKENIAQLTGYTGTHRLSLKCIRLVLEEQWYSSRNQMEIFTHLNIKPKKINLTAANKIPKAMIDEFILSPVVKRTFGQAINLINKIIEKYGVPEDIIIELARENNSKDKQKFINEMQKKNENTRKRINEIIGKYGNQNAKRLVEKIRLHDEQEGKCLYSLESIPLEDLLNNPNHYEVDHIIPRSVSFDNSYHNKVLVKQSEASKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIESKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 1011 - protein, 1053 aa, synthetic construct
NQKFILGLDIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGKGSPYGWEGDPKAWYETLMGHCTYFPDELRSVKYAYSADLFNALNDLNNLVIQRDGLSKLEYHEKYHIIENVFKQKKKPTLKQIANEINVNPEDIKGYRITKSGKPQFTEFKLYHDLKSVLFDQSILENEDVLDQIAEILTIYQDKDSIKSKLTELDILLNEEDKENIAQLTGYTGTHRLSLKCIRLVLEEQWYSSRNQMEIFTHLNIKPKKINLTAANKIPKAMIDEFILSPVVKRTFGQAINLINKIIEKYGVPEDIIIELARENNSKDKQKFINEMQKKNENTRKRINEIIGKYGNQNAKRLVEKIRLHDEQEGKCLYSLESIPLEDLLNNPNHYEVDHIIPRSVSFDNSYHNKVLVKQSEASKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIESKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 1012 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 1013 - protein, 1053 aa, synthetic construct
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 1014 - protein, 1052 aa, synthetic construct
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 1015 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKVYRSTKEVLDATLIHQSITGLYETRIDLSQLGGD

SEQ ID NO: 1016 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKVYRSTKEVLDATLIHQSITGLYETRIDLSQLGGD

SEQ ID NO: 1017 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKVYRSTKEVLDATLIHQSITGLYETRIDLSQLGGD

SEQ ID NO: 1018 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 1019 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 1020 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 1021 - protein, 1368 aa, synthetic construct
MDKKYSIGLDIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGD

SEQ ID NO: 1022 - protein, 1368 aa, synthetic construct
MDKKYSIGLDIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGD

SEQ ID NO: 1023 - protein, 1367 aa, synthetic construct
DKKYSIGLDIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGD

SEQ ID NO: 1024 - protein, 1057 aa, synthetic construct
MNQKFILGLDIGITSVGYGLIDYETKNIIDAGVRLFPEANVENNEGRRSKRGSRRLKRRRIHRLERVKLLLTEYDLINKEQIPTSNNPYQIRVKGLSEILSKDELAIALLHLAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 1025 - protein, 1057 aa, synthetic construct
MNQKFILGLDIGITSVGYGLIDYETKNIIDAGVRLFPEANVENNEGRRSKRGSRRLKRRRIHRLERVKLLLTEYDLINKEQIPTSNNPYQIRVKGLSEILSKDELAIALLHLAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSEASKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 1026 - protein, 1056 aa, synthetic construct
NQKFILGLDIGITSVGYGLIDYETKNIIDAGVRLFPEANVENNEGRRSKRGSRRLKRRRIHRLERVKLLLTEYDLINKEQIPTSNNPYQIRVKGLSEILSKDELAIALLHLAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSEASKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 1027 - protein, 1057 aa, synthetic construct
MNQKFILGLDIGITSVGYGLIDYETKNIIDAGVRLFPEANVENNEGRRSKRGSRRLKRRRIHRLERVKLLLTEYDLINKEQIPTSNNPYQIRVKGLSEILSKDELAIALLHLAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTSYLKAYFSANNMDVKVKTINGSFTNHLRKVWRFDKYRNHGYKHHAEDALIIANADFLFKENKKLQNTNKILEKPTIENNTKKVTVEKEEDYNNVFETPKLVEDIKQYRDYKFSHRVDKKPNRQLINDTLYSTRMKDEHDYIVQTITDIYGKDNTNLKKQFNKNPEKFLMYQNDPKTFEKLSIIMKQYSDEKNPLAKYYEETGEYLTKYSKKNNGPIVKKIKLLGNKVGNHLDVTNKYENSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 1028 - protein, 1057 aa, synthetic construct
MNQKFILGLDIGITSVGYGLIDYETKNIIDAGVRLFPEANVENNEGRRSKRGSRRLKRRRIHRLERVKLLLTEYDLINKEQIPTSNNPYQIRVKGLSEILSKDELAIALLHLAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSEASKKSNLTPYQYFNSGKSKLSYNQFKQHILNLSKSQDRISKKKKEYLLEERDINKFEVQKEFINRNLVDTRYATRELTSYLKAYFSANNMDVKVKTINGSFTNHLRKVWRFDKYRNHGYKHHAEDALIIANADFLFKENKKLQNTNKILEKPTIENNTKKVTVEKEEDYNNVFETPKLVEDIKQYRDYKFSHRVDKKPNRQLINDTLYSTRMKDEHDYIVQTITDIYGKDNTNLKKQFNKNPEKFLMYQNDPKTFEKLSIIMKQYSDEKNPLAKYYEETGEYLTKYSKKNNGPIVKKIKLLGNKVGNHLDVTNKYENSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 1029 - protein, 1056 aa, synthetic construct
NQKFILGLDIGITSVGYGLIDYETKNIIDAGVRLFPEANVENNEGRRSKRGSRRLKRRRIHRLERVKLLLTEYDLINKEQIPTSNNPYQIRVKGLSEILSKDELAIALLHLAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSEASKKSNLTPYQYFNSGKSKLSYNQFKQHILNLSKSQDRISKKKKEYLLEERDINKFEVQKEFINRNLVDTRYATRELTSYLKAYFSANNMDVKVKTINGSFTNHLRKVWRFDKYRNHGYKHHAEDALIIANADFLFKENKKLQNTNKILEKPTIENNTKKVTVEKEEDYNNVFETPKLVEDIKQYRDYKFSHRVDKKPNRQLINDTLYSTRMKDEHDYIVQTITDIYGKDNTNLKKQFNKNPEKFLMYQNDPKTFEKLSIIMKQYSDEKNPLAKYYEETGEYLTKYSKKNNGPIVKKIKLLGNKVGNHLDVTNKYENSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 1030 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 1031 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 1032 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 1033 - protein, 2117 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 1034 - protein, 19 aa, synthetic construct
MKRTADGSEFESPKKKRKV

SEQ ID NO: 1035 - skipped

SEQ ID NO: 1036 - protein, 33 aa, synthetic construct
SGGSSGGSSGSETPGTSESATPESSGGSSGGSS

SEQ ID NO: 1037 - skipped

SEQ ID NO: 1038 - protein, 21 aa, synthetic construct
SGGSKRTADGSEFEPKKKRKV

SEQ ID NO: 1039 - protein, 2131 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFESPKKKRKVGSGPAAKRVKLD

SEQ ID NO: 1040-1041 - skipped (2 entries)

SEQ ID NO: 1042 - protein, 34 aa, synthetic construct
SGGSSGGSKRTADGSEFESPKKKRKVSGGSSGGS

SEQ ID NO: 1043 - skipped

SEQ ID NO: 1044 - protein, 22 aa, synthetic construct
SGGSKRTADGSEFESPKKKRKV

SEQ ID NO: 1045 - protein, 12 aa, synthetic construct
GSGPAAKRVKLD

SEQ ID NO: 1046 - DNA, 43 bp, synthetic construct
  source 1..43: mol_type = other DNA
ggcttgtcgacgacggcggtctcagtggtgtacggtacaaacc

SEQ ID NO: 1047 - DNA, 43 bp, synthetic construct
  source 1..43: mol_type = other DNA
ggtttgtctggtcaaccaccgcggtctccgtcgtcaggatcat

SEQ ID NO: 1048-1059 - skipped (12 entries)

SEQ ID NO: 1060 - RNA, 20 nt, synthetic construct
  source 1..20: mol_type = other RNA
actcacgctggatagcctcc

SEQ ID NO: 1061 - RNA, 19 nt, synthetic construct
  source 1..19: mol_type = other RNA
ctcacgctggatagcctcc

SEQ ID NO: 1062 - RNA, 18 nt, synthetic construct
  source 1..18: mol_type = other RNA
tcacgctggatagcctcc

SEQ ID NO: 1063 - RNA, 17 nt, synthetic construct
  source 1..17: mol_type = other RNA
cacgctggatagcctcc

SEQ ID NO: 1064-1068 - skipped (5 entries)

SEQ ID NO: 1069 - RNA, 10 nt, synthetic construct
  source 1..10: mol_type = other RNA
ggctatccag

SEQ ID NO: 1070 - RNA, 11 nt, synthetic construct
  source 1..11: mol_type = other RNA
ggctatccagc

SEQ ID NO: 1071 - RNA, 12 nt, synthetic construct
  source 1..12: mol_type = other RNA
ggctatccagcg

SEQ ID NO: 1072 - RNA, 13 nt, synthetic construct
  source 1..13: mol_type = other RNA
ggctatccagcgt

SEQ ID NO: 1073 - RNA, 14 nt, synthetic construct
  source 1..14: mol_type = other RNA
ggctatccagcgtg

SEQ ID NO: 1074 - RNA, 15 nt, synthetic construct
  source 1..15: mol_type = other RNA
ggctatccagcgtga

SEQ ID NO: 1075 - RNA, 16 nt, synthetic construct
  source 1..16: mol_type = other RNA
ggctatccagcgtgag

SEQ ID NO: 1076 - RNA, 17 nt, synthetic construct
  source 1..17: mol_type = other RNA
ggctatccagcgtgagt

SEQ ID NO: 1077 - RNA, 16 nt, synthetic construct
  source 1..16: mol_type = other RNA
ctggcctgtaataaga

SEQ ID NO: 1078 - RNA, 20 nt, synthetic construct
  source 1..20: mol_type = other RNA
ctttctggcctgtaataaga

SEQ ID NO: 1079 - RNA, 24 nt, synthetic construct
  source 1..24: mol_type = other RNA
ctctctttctggcctgtaataaga

SEQ ID NO: 1080 - RNA, 16 nt, synthetic construct
  source 1..16: mol_type = other RNA
tctctttctggctgga

SEQ ID NO: 1081 - RNA, 10 nt, synthetic construct
  source 1..10: mol_type = other RNA
tctggctgga

SEQ ID NO: 1082 - RNA, 12 nt, synthetic construct
  source 1..12: mol_type = other RNA
tttctggctgga

SEQ ID NO: 1083 - RNA, 14 nt, synthetic construct
  source 1..14: mol_type = other RNA
tctttctggctgga

SEQ ID NO: 1084 - RNA, 18 nt, synthetic construct
  source 1..18: mol_type = other RNA
tctctctttctggctgga

SEQ ID NO: 1085 - RNA, 20 nt, synthetic construct
  source 1..20: mol_type = other RNA
actctctctttctggctgga

SEQ ID NO: 1086 - RNA, 11 nt, synthetic construct
  source 1..11: mol_type = other RNA
ctgggcctgga

SEQ ID NO: 1087 - RNA, 13 nt, synthetic construct
  source 1..13: mol_type = other RNA
ttctgggcctgga

SEQ ID NO: 1088 - RNA, 15 nt, synthetic construct
  source 1..15: mol_type = other RNA
ctttctgggcctgga

SEQ ID NO: 1089 - RNA, 17 nt, synthetic construct
  source 1..17: mol_type = other RNA
ctctttctgggcctgga

SEQ ID NO: 1090 - RNA, 128 nt, synthetic construct
  source 1..128: mol_type = other RNA
actcacgctggatagcctccgttttagagctagaaatagcaagttaaaataaggctagtccgttatcaacttgaaaaagtggcaccgagtcggtgctctctttctggctggaggctatccagcgtttt

SEQ ID NO: 1091 - RNA, 122 nt, synthetic construct
  source 1..122: mol_type = other RNA
actcacgctggatagcctccgttttagagctagaaatagcaagttaaaataaggctagtccgttatcaacttgaaaaagtggcaccgagtcggtgctctggctggaggctatccagcgtttt

SEQ ID NO: 1092 - RNA, 124 nt, synthetic construct
  source 1..124: mol_type = other RNA
actcacgctggatagcctccgttttagagctagaaatagcaagttaaaataaggctagtccgttatcaacttgaaaaagtggcaccgagtcggtgctttctggctggaggctatccagcgtttt

SEQ ID NO: 1093 - RNA, 126 nt, synthetic construct
  source 1..126: mol_type = other RNA
actcacgctggatagcctccgttttagagctagaaatagcaagttaaaataaggctagtccgttatcaacttgaaaaagtggcaccgagtcggtgctctttctggctggaggctatccagcgtttt

SEQ ID NO: 1094 - RNA, 130 nt, synthetic construct
  source 1..130: mol_type = other RNA
actcacgctggatagcctccgttttagagctagaaatagcaagttaaaataaggctagtccgttatcaacttgaaaaagtggcaccgagtcggtgctctctctttctggctggaggctatccagcgtttt

SEQ ID NO: 1095 - RNA, 132 nt, synthetic construct
  source 1..132: mol_type = other RNA
actcacgctggatagcctccgttttagagctagaaatagcaagttaaaataaggctagtccgttatcaacttgaaaaagtggcaccgagtcggtgcactctctctttctggctggaggctatccagcgtttt

SEQ ID NO: 1096 - RNA, 127 nt, synthetic construct
  source 1..127: mol_type = other RNA
actcacgctggatagcctccgttttagagctagaaatagcaagttaaaataaggctagtccgttatcaacttgaaaaagtggcaccgagtcggtgcctttctgggcctggaggctatccagcgtttt

SEQ ID NO: 1097 - RNA, 119 nt, synthetic construct
  source 1..119: mol_type = other RNA
actcacgctggatagcctccgttttagagctagaaatagcaagttaaaataaggctagtccgttatcaacttgaaaaagtggcaccgagtcggtgcctgggcctggaggctatcctttt

SEQ ID NO: 1098 - RNA, 121 nt, synthetic construct
  source 1..121: mol_type = other RNA
actcacgctggatagcctccgttttagagctagaaatagcaagttaaaataaggctagtccgttatcaacttgaaaaagtggcaccgagtcggtgcttctgggcctggaggctatcctttt

SEQ ID NO: 1099 - RNA, 123 nt, synthetic construct
  source 1..123: mol_type = other RNA
actcacgctggatagcctccgttttagagctagaaatagcaagttaaaataaggctagtccgttatcaacttgaaaaagtggcaccgagtcggtgcctttctgggcctggaggctatcctttt

SEQ ID NO: 1100 - RNA, 125 nt, synthetic construct
  source 1..125: mol_type = other RNA
actcacgctggatagcctccgttttagagctagaaatagcaagttaaaataaggctagtccgttatcaacttgaaaaagtggcaccgagtcggtgcctctttctgggcctggaggctatcctttt

SEQ ID NO: 1101 - RNA, 121 nt, synthetic construct
  source 1..121: mol_type = other RNA
actcacgctggatagcctccgttttagagctagaaatagcaagttaaaataaggctagtccgttatcaacttgaaaaagtggcaccgagtcggtgcctgggcctggaggctatccagtttt

SEQ ID NO: 1102 - RNA, 123 nt, synthetic construct
  source 1..123: mol_type = other RNA
actcacgctggatagcctccgttttagagctagaaatagcaagttaaaataaggctagtccgttatcaacttgaaaaagtggcaccgagtcggtgcttctgggcctggaggctatccagtttt

SEQ ID NO: 1103 - RNA, 125 nt, synthetic construct
  source 1..125: mol_type = other RNA
actcacgctggatagcctccgttttagagctagaaatagcaagttaaaataaggctagtccgttatcaacttgaaaaagtggcaccgagtcggtgcctttctgggcctggaggctatccagtttt

SEQ ID NO: 1104 - RNA, 127 nt, synthetic construct
  source 1..127: mol_type = other RNA
actcacgctggatagcctccgttttagagctagaaatagcaagttaaaataaggctagtccgttatcaacttgaaaaagtggcaccgagtcggtgcctctttctgggcctggaggctatccagtttt

SEQ ID NO: 1105 - RNA, 123 nt, synthetic construct
  source 1..123: mol_type = other RNA
actcacgctggatagcctccgttttagagctagaaatagcaagttaaaataaggctagtccgttatcaacttgaaaaagtggcaccgagtcggtgcctgggcctggaggctatccagcgtttt

SEQ ID NO: 1106 - RNA, 125 nt, synthetic construct
  source 1..125: mol_type = other RNA
actcacgctggatagcctccgttttagagctagaaatagcaagttaaaataaggctagtccgttatcaacttgaaaaagtggcaccgagtcggtgcttctgggcctggaggctatccagcgtttt

SEQ ID NO: 1107 - RNA, 129 nt, synthetic construct
  source 1..129: mol_type = other RNA
actcacgctggatagcctccgttttagagctagaaatagcaagttaaaataaggctagtccgttatcaacttgaaaaagtggcaccgagtcggtgcctctttctgggcctggaggctatccagcgtttt

SEQ ID NO: 1108 - RNA, 125 nt, synthetic construct
  source 1..125: mol_type = other RNA
actcacgctggatagcctccgttttagagctagaaatagcaagttaaaataaggctagtccgttatcaacttgaaaaagtggcaccgagtcggtgcctgggcctggaggctatccagcgtgtttt

SEQ ID NO: 1109 - RNA, 127 nt, synthetic construct
  source 1..127: mol_type = other RNA
actcacgctggatagcctccgttttagagctagaaatagcaagttaaaataaggctagtccgttatcaacttgaaaaagtggcaccgagtcggtgcttctgggcctggaggctatccagcgtgtttt

SEQ ID NO: 1110 - RNA, 129 nt, synthetic construct
  source 1..129: mol_type = other RNA
actcacgctggatagcctccgttttagagctagaaatagcaagttaaaataaggctagtccgttatcaacttgaaaaagtggcaccgagtcggtgcctttctgggcctggaggctatccagcgtgtttt

SEQ ID NO: 1111 - RNA, 131 nt, synthetic construct
  source 1..131: mol_type = other RNA
actcacgctggatagcctccgttttagagctagaaatagcaagttaaaataaggctagtccgttatcaacttgaaaaagtggcaccgagtcggtgcctctttctgggcctggaggctatccagcgtgtttt

SEQ ID NO: 1112 - RNA, 121 nt, synthetic construct
  source 1..121: mol_type = other RNA
actcacgctggatagcctccgtttaagagctagaaatagcaagtttaaataaggctagtccgttatcagcgtgaaaacgcggcaccgagtcggtgcctggcctgtaataagaggctatcca

SEQ ID NO: 1113 - RNA, 125 nt, synthetic construct
  source 1..125: mol_type = other RNA
actcacgctggatagcctccgtttaagagctagaaatagcaagtttaaataaggctagtccgttatcagcgtgaaaacgcggcaccgagtcggtgcctttctggcctgtaataagaggctatcca

SEQ ID NO: 1114 - RNA, 129 nt, synthetic construct
  source 1..129: mol_type = other RNA
actcacgctggatagcctccgtttaagagctagaaatagcaagtttaaataaggctagtccgttatcagcgtgaaaacgcggcaccgagtcggtgcctctctttctggcctgtaataagaggctatcca

SEQ ID NO: 1115 - RNA, 124 nt, synthetic construct
  source 1..124: mol_type = other RNA
actcacgctggatagcctccgtttaagagctagaaatagcaagtttaaataaggctagtccgttatcagcgtgaaaacgcggcaccgagtcggtgcctggcctgtaataagaggctatccagcg

SEQ ID NO: 1116 - RNA, 128 nt, synthetic construct
  source 1..128: mol_type = other RNA
actcacgctggatagcctccgtttaagagctagaaatagcaagtttaaataaggctagtccgttatcagcgtgaaaacgcggcaccgagtcggtgcctttctggcctgtaataagaggctatccagcg

SEQ ID NO: 1117 - RNA, 132 nt, synthetic construct
  source 1..132: mol_type = other RNA
actcacgctggatagcctccgtttaagagctagaaatagcaagtttaaataaggctagtccgttatcagcgtgaaaacgcggcaccgagtcggtgcctctctttctggcctgtaataagaggctatccagcg

SEQ ID NO: 1118 - RNA, 127 nt, synthetic construct
  source 1..127: mol_type = other RNA
actcacgctggatagcctccgtttaagagctagaaatagcaagtttaaataaggctagtccgttatcagcgtgaaaacgcggcaccgagtcggtgcctggcctgtaataagaggctatccagcgtga

SEQ ID NO: 1119 - RNA, 131 nt, synthetic construct
  source 1..131: mol_type = other RNA
actcacgctggatagcctccgtttaagagctagaaatagcaagtttaaataaggctagtccgttatcagcgtgaaaacgcggcaccgagtcggtgcctttctggcctgtaataagaggctatccagcgtga

SEQ ID NO: 1120 - RNA, 135 nt, synthetic construct
  source 1..135: mol_type = other RNA
actcacgctggatagcctccgtttaagagctagaaatagcaagtttaaataaggctagtccgttatcagcgtgaaaacgcggcaccgagtcggtgcctctctttctggcctgtaataagaggctatccagcgtga

SEQ ID NO: 1121 - RNA, 20 nt, synthetic construct
  source 1..20: mol_type = other RNA
ggccgagatgtctcgctccg

SEQ ID NO: 1122 - RNA, 20 nt, synthetic construct
  source 1..20: mol_type = other RNA
tcctgaagctgacagcattc

SEQ ID NO: 1123 - RNA, 20 nt, synthetic construct
  source 1..20: mol_type = other RNA
ttcctgaagctgacagcatt

SEQ ID NO: 1124 - RNA, 20 nt, synthetic construct
  source 1..20: mol_type = other RNA
tactctctctttctggctgg

SEQ ID NO: 1125 - RNA, 20 nt, synthetic construct
  source 1..20: mol_type = other RNA
ctctctctttctgggcctgg

SEQ ID NO: 1126 - RNA, 19 nt, synthetic construct
  source 1..19: mol_type = other RNA
ctactctctctttctgggc

SEQ ID NO: 1127 - RNA, 147 nt, synthetic construct
  source 1..147: mol_type = other RNA
agagtctctcagctggtacagttttagagctagaaatagcaagttaaaataaggctagtccgttatcaacttgaaaaagtggcaccgagtcggtgcatgatcctgacgacggagaccgccgtcgtcgacaagccaccagctgatttt

SEQ ID NO: 1128 - RNA, 145 nt, synthetic construct
  source 1..145: mol_type = other RNA
gctggtacacggcagggtcagttttagagctagaaatagcaagttaaaataaggctagtccgttatcaacttgaaaaagtggcaccgagtcggtgcatgatcctgacgacggagaccgccgtcgtcgacaagccccctgcctttt

SEQ ID NO: 1129 - RNA, 95 nt, synthetic construct
  source 1..95: mol_type = other RNA
ctactctctctttctgggcgttttagagctagaaatagcaagttaaaataaggctagtccgttatcaacttgaaaaagtggcaccgagtcggtgc

SEQ ID NO: 1130 - RNA, 99 nt, synthetic construct
  source 1..99: mol_type = other RNA
ctactctctctttctgggcgttttagagctagaaatagcaagttaaaataaggctagtccgttatcaacttgaaaaagtggcaccgagtcggtgctttt

SEQ ID NO: 1131 - protein, 500 aa, synthetic construct [sequence omitted]

SEQ ID NO: 1132 - RNA, 139 nt, synthetic construct
  source 1..139: mol_type = other RNA
ctggactccagcctgggttggttttagagctagaaatagcaagttaaaataaggctagtccgttatcaacttgaaaaagtggcaccgagtcggtgcacgacggagaccgccgtcgtcgacaagcccccaggctggtttt

SEQ ID NO: 1133-1134 - skipped (2 entries)

SEQ ID NO: 1135 - RNA, 141 nt, synthetic construct
  source 1..141: mol_type = other RNA
tctctcagctggtacacggcgttttagagctagaaatagcaagttaaaataaggctagtccgttatcaacttgaaaaagtggcaccgagtcggtgcgacgacggagaccgccgtcgtcgacaagccgtgtaccagcttttt

SEQ ID NO: 1136 - RNA, 141 nt, synthetic construct
  source 1..141: mol_type = other RNA
agagtctctcagctggtacagttttagagctagaaatagcaagttaaaataaggctagtccgttatcaacttgaaaaagtggcaccgagtcggtgcgacgacggagaccgccgtcgtcgacaagccaccagctgagatttt

SEQ ID NO: 1137 - RNA, 137 nt, synthetic construct
  source 1..137: mol_type = other RNA
gctggtacacggcagggtcagttttagagctagaaatagcaagttaaaataaggctagtccgttatcaacttgaaaaagtggcaccgagtcggtgcgacgacggagaccgccgtcgtcgacaagccccctgcctttt

SEQ ID NO: 1138 - RNA, 139 nt, synthetic construct
  source 1..139: mol_type = other RNA
gctggtacacggcagggtcagttttagagctagaaatagcaagttaaaataaggctagtccgttatcaacttgaaaaagtggcaccgagtcggtgcgacgacggagaccgccgtcgtcgacaagccccctgccgttttt

SEQ ID NO: 1139 - RNA, 142 nt, synthetic construct
  source 1..142: mol_type = other RNA
agagtctctcagctggtacagttttagagctagaaatagcaagttaaaataaggctagtccgttatcaacttgaaaaagtggcaccgagtcggtgcacgacggcggtctccgtcgtcaggatcataccagctgagagatttt

SEQ ID NO: 1140 - RNA, 137 nt, synthetic construct
  source 1..137: mol_type = other RNA
gctggtacacggcagggtcagttttagagctagaaatagcaagttaaaataaggctagtccgttatcaacttgaaaaagtggcaccgagtcggtgcacgacggcggtctccgtcgtcaggatcatccctgccgtttt

SEQ ID NO: 1141 - RNA, 140 nt, synthetic construct
  source 1..140: mol_type = other RNA
agagtctctcagctggtacagttttagagctagaaatagcaagttaaaataaggctagtccgttatcaacttgaaaaagtggcaccgagtcggtgcacgacggcggtctccgtcgtcaggatcataccagctgagatttt

SEQ ID NO: 1142 - RNA, 142 nt, synthetic construct
  source 1..142: mol_type = other RNA
tctctcagctggtacacggcgttttagagctagaaatagcaagttaaaataaggctagtccgttatcaacttgaaaaagtggcaccgagtcggtgcacgacggcggtctccgtcgtcaggatcatgtgtaccagctgatttt

SEQ ID NO: 1143-1152 - skipped (10 entries)

SEQ ID NO: 1153 - protein, 30 aa, synthetic construct
MDSLLMNRRKFLYQFKNVRWAKGRRETYLC

SEQ ID NO: 1154 - protein, 18 aa, synthetic construct
KRTADGSEFESPKKKRKV

SEQ ID NO: 1155 - protein, 17 aa, synthetic construct
KRTADGSEFEPKKKRKV

SEQ ID NO: 1156 - protein, 19 aa, synthetic construct
NLSKRPAAIKKAGQAKKKK

SEQ ID NO: 1157 - protein, 11 aa, synthetic construct
RQRRNELKRSF

SEQ ID NO: 1158 - protein, 38 aa, synthetic construct
NQSSNFGPMKGGNFGGRSSGPYGGGGQYFAKPRNQGGY

SEQ ID NO: 1159 - protein, 7 aa, synthetic construct
PKKKRKV

SEQ ID NO: 1160 - protein, 16 aa, synthetic construct
  VARIANT 3..12: note = Any amino acid
KRXXXXXXXXXXKKKL

SEQ ID NO: 1161 - protein, 16 aa, synthetic construct
KRPAATKKAGQAKKKK

SEQ ID NO: 1162 - protein, 20 aa, synthetic construct
AVKRPAATKKAGQAKKKKLD

SEQ ID NO: 1163 - protein, 25 aa, synthetic construct
MSRRRKANPTKLSENAKKLAKEVEN

SEQ ID NO: 1164 - protein, 9 aa, synthetic construct
PAAKRVKLD

SEQ ID NO: 1165 - protein, 9 aa, synthetic construct
KLKIKRPVK

SEQ ID NO: 1166 - protein, 8 aa, synthetic construct
VSRKRPRP

SEQ ID NO: 1167 - protein, 10 aa, synthetic construct
EGAPPAKRAR

SEQ ID NO: 1168 - protein, 12 aa, synthetic construct
PPQPKKKPLDGE

SEQ ID NO: 1169 - RNA, 33 nt, synthetic construct
  source 1..33: mol_type = other RNA
gccaacatgaggatcacccatgtctgcagggcc

SEQ ID NO: 1170 - protein, 131 aa, synthetic construct
GSASNFTQFVLVDNGGTGDVTVAPSNFANGVAEWISSNSRSQAYKVTCSVRQSSAQNRKYTIKVEVPKVATQTVGGEELPVAGWRSYLNMELTIPIFATNSDCELIVKAMQGLLKDGNPIPSAIAANSGIY

SEQ ID NO: 1171 - protein, 150 aa, synthetic construct
  VARIANT 6..150: note = GGGGS repeats may be deleted
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 1172 - protein, 30 aa, synthetic construct
  VARIANT 2..30: note = Residues may be deleted
GGGGGGGGGGGGGGGGGGGGGGGGGGGGGG

SEQ ID NO: 1173 - protein, 150 aa, synthetic construct
  VARIANT 6..150: note = EAAAK repeats may be deleted
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 1174 - protein, 90 aa, synthetic construct
  VARIANT 4..90: note = GGS repeats may be deleted
GGSGGSGGSGGSGGSGGSGGSGGSGGSGGSGGSGGSGGSGGSGGSGGSGGSGGSGGSGGSGGSGGSGGSGGSGGSGGSGGSGGSGGSGGS

SEQ ID NO: 1175 - protein, 120 aa, synthetic construct
  VARIANT 5..120: note = SGGS repeats may be deleted
SGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGS

SEQ ID NO: 1176 - protein, 60 aa, synthetic construct
  VARIANT 3..60: note = XP repeats may be deleted and wherein X is any amino acid
XPXPXPXPXPXPXPXPXPXPXPXPXPXPXPXPXPXPXPXPXPXPXPXPXPXPXPXPXPXP

SEQ ID NO: 1177 - protein, 21 aa, synthetic construct
  VARIANT 4..21: note = 4 or 6 GGS repeats may be deleted
GGSGGSGGSGGSGGSGGSGGS

SEQ ID NO: 1178 - protein, 16 aa, synthetic construct
SGSETPGTSESATPES

SEQ ID NO: 1179 - protein, 32 aa, synthetic construct
SGGSSGGSSGSETPGTSESATPESSGGSSGGS

SEQ ID NO: 1180 - protein, 10 aa, synthetic construct
SGGSGGSGGS

SEQ ID NO: 1181 - protein, 4 aa, synthetic construct
SGGS

SEQ ID NO: 1182 - protein, 60 aa, synthetic construct
SGGSSGGSSGSETPGTSESATPESAGSYPYDVPDYAGSAAPAAKKKKLDGSGSGGSSGGS

SEQ ID NO: 1183 - protein, 6 aa, synthetic construct
GGSGGS

SEQ ID NO: 1184 - protein, 9 aa, synthetic construct
GGSGGSGGS

SEQ ID NO: 1185 - protein, 140 aa, Homo sapiens
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 1186 - DNA, 1508 bp, Homo sapiens
  source 1..1508: mol_type = unassigned DNA
ttttgaaacccttcaaaggcagagacttgtccagcctaacctgcctgctgctcctagctcctgaggctcagggcccttggcttctgtccgctctgctcagggccctccagcgtggccactgctcagccatgctcctgctgctcgtcccagtgctcgaggtgatttttaccctgggaggaaccagagcccagtcggtgacccagcttggcagccacgtctctgtctctgaaggagccctggttctgctgaggtgcaactactcatcgtctgttccaccatatctcttctggtatgtgcaataccccaaccaaggactccagcttctcctgaagtacacatcagcggccaccctggttaaaggcatcaacggttttgaggctgaatttaagaagagtgaaacctccttccacctgacgaaaccctcagcccatatgagcgacgcggctgagtacttctgtgctgtgagtgatctcgaaccgaacagcagtgcttccaagataatctttggatcagggaccagactcagcatccggccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctgagatctgcaagattgtaagacagcctgtgctccctcgctccttcctctgcattgcccctcttctccctctccaaacagagggaactctcctacccccaaggaggtgaaagctgctaccacctctgtgcccccccggtaatgccaccaactggatcctacccgaatttatgattaagattgctgaagagctgccaaacactgctgccaccccctctgttcccttattgctgcttgtcactgcctgacattcacggcagaggcaaggctgctgcagcctcccctggctgtgcacattccctcctgctccccagagactgcctccgccatcccacagatgatggatcttcagtgggttctcttgggctctaggtcctggagaatgttgtgaggggtttatttttttttaatagtgttcataaagaaatacatagtattcttcttctcaagacgtggggggaaattatctcattatcgaggccctgctatgctgtgtgtctgggcgtgttgtatgtcctgctgccgatgccttcattaaaatgatttggaa

SEQ ID NO: 1187 - DNA, 38 bp, synthetic construct
  source 1..38: mol_type = other DNA
ggcttgtcgacgacggcggtctccgtcgtcaggatcat

SEQ ID NO: 1188 - DNA, 48 bp, synthetic construct
  source 1..48: mol_type = other DNA
ggtttgtctggtcaaccaccgcggtctcagtggtgtacggtacaaacc

SEQ ID NO: 1189 - DNA, 38 bp, synthetic construct
  source 1..38: mol_type = other DNA
ggcttgtcgacgacggcggactccgtcgtcaggatcat

SEQ ID NO: 1190 - DNA, 48 bp, synthetic construct
  source 1..48: mol_type = other DNA
ggtttgtctggtcaaccaccgcggactcagtggtgtacggtacaaacc

SEQ ID NO: 1191 - DNA, 104 bp, synthetic construct
  source 1..104: mol_type = other DNA
ccggtttcccttcgcacccgcaccgcggcttcgagaccgtgacctacatgctcgaagggcgtatgcgccacgaagaccacctcggcaatcgcggcctgctcaag

SEQ ID NO: 1192 - DNA, 48 bp, synthetic construct; identical to SEQ ID NO: 1190
  source 1..48: mol_type = other DNA

SEQ ID NO: 1193 - DNA, 33 bp, synthetic construct
  source 1..33: mol_type = other DNA
ccgtgacctacatgctcgaagggcgtatgcgcc

SEQ ID NO: 1194 - DNA, 104 bp, synthetic construct
  source 1..104: mol_type = other DNA
gtaacgctcttcgagaaagcagattctcatatccatcttgagtcttctttctcgcaagacaacacgaaatagacacagtctcttccctagctgtacactgagcc

SEQ ID NO: 1195 - DNA, 112 bp, synthetic construct
  source 1..112: mol_type = other DNA
ttccgacgcagtttccgacgagtacgaggacgaggacagacgtgcctaccggcaaggtcaagtggttcaacagcgagaagggcttcggctttctctcccgcgacgacggcgg

SEQ ID NO: 1196 - DNA, 112 bp, synthetic construct
  source 1..112: mol_type = other DNA
tagtgacgtctgtccgcgcagtgatcgagggagtgtgtgctttgccgactggcaaggtcaagccggtctgctaggcacagagagccggtacagtcctccccatgcaacccaa

SEQ ID NO: 1197 - DNA, 115 bp, synthetic construct
  source 1..115: mol_type = other DNA
gtgacccgttccgccgtcgagaacgccacctccgtggcgcgcatggtgctcaccaccgagagcgccgtggtcgacaagccggccgaggaagagcccgcgaacggccaccacggcc

SEQ ID NO: 1198 - DNA, 115 bp, synthetic construct
  source 1..115: mol_type = other DNA
tgattacgatcagtgccctgggaggcgattccggcatggcttatatccaacaccaccgagagcgctgttgtcgagcgtgtaagccaggacgaggacgagcacgcccacgggcacg

SEQ ID NO: 1199 - DNA, 113 bp, synthetic construct
  source 1..113: mol_type = other DNA
tttgcatttaaaatcggagcatcatttttcaacagaaacgactatgagcgcaatgacccttgatttacctcgccgctttccgtggccaacgctgctgtccgtggctatccacg

SEQ ID NO: 1200 - DNA, 113 bp, synthetic construct
  source 1..113: mol_type = other DNA
agaaccttgaaaaactatggcttatgctacctcgccgaatagctcaaatacaatgacccttgaatataggctattcgggttttgaaggtatctggtttttattcacatcacaa

SEQ ID NO: 1201 - DNA, 117 bp, synthetic construct
  source 1..117: mol_type = other DNA
cgaaatggttggcgttgaggtcaatgattaatgtgtatagggttaacatttaaatcagtacaatcgtagacgctctacactattttctgtgtataaaaatatcgagaataaacgctt

SEQ ID NO: 1202 - DNA, 117 bp, synthetic construct
  source 1..117: mol_type = other DNA
gctggtcgaggagttaatgaagatctccaacatgttcggctacggtgaggtaaatcagtacaatcgtcactcgaccctaatgtctgcctataaaaacatcaaagatgaacatttccg

SEQ ID NO: 1203 - DNA, 38 bp, synthetic construct
  source 1..38: mol_type = other DNA
gatatggggaagtgaatcagtacaaccgccacagtacc

SEQ ID NO: 1204 - DNA, 57 bp, synthetic construct
  source 1..57: mol_type = other DNA
ttcgggtgctgggttgttgtctctggacagtgatccatgggaaactactcagcacca

SEQ ID NO: 1205 - DNA, 50 bp, synthetic construct
  source 1..50: mol_type = other DNA
attgttgttgtttttccagatccagttggtcctgtaaatataagcaatcc

SEQ ID NO: 1206 - DNA, 50 bp, synthetic construct
  source 1..50: mol_type = other DNA
ggttgtatttgtagaacttgaccagttgttttagtaacataaatacaact

SEQ ID NO: 1207 - DNA, 50 bp, synthetic construct
  source 1..50: mol_type = other DNA
cattatatgtttttacaatccgggccgccatactgtaagaacatataatg

SEQ ID NO: 1208 - DNA, 38 bp, synthetic construct; identical to SEQ ID NO: 1203
  source 1..38: mol_type = other DNA

SEQ ID NO: 1209 - DNA, 50 bp, synthetic construct
  source 1..50: mol_type = other DNA
cgttatagggtattgcagtaccgaccgccatactgtaataccctataacg

SEQ ID NO: 1210 - DNA, 50 bp, synthetic construct
  source 1..50: mol_type = other DNA
gatgcactgagctcaccgtccggaccgccatactgacttatgatataaga

SEQ ID NO: 1211 - skipped

SEQ ID NO: 1212 - protein, 555 aa, synthetic construct
MQPNLRYLACLRLSADSDGSTSIEWQRGVIRHHVSSPHLSGVLVGEAEDTDVSGSLSPFKRPKLGKWLTAKADEFDVIIAAKMDRLTRRSMHFNELLEWAQQNGKFIVCVEEGFDLSTPQGKMMARMTAVFAEAEWDTIQARILNGVQTRLENRSWLVGAPPTGYRIKTVEGGKRKILEIDQDFYPYVEEIFRRIREGQSTHRIARDFNGRSVLTWGDHLRKLKGEEPKGTQWQATIINKFIRSSWVPGLYTYKGEAVLDDQGDPVILPETPLATMDEWTDLVDRIKPAPKPEGATGGSRNSAKSLLSGVAHCGECGAPFTSLMDSGYKRKDGTKVPGHRRYRCSNKFKGGDCKNGSYVRADVLDSWVDQAIRDSIGQEDMYERAGKGPSQARELQETKARLAKLEADYESGKYDGEGQDESYWRMNKNLSAKVAHLAKQEAERANPTFKATGKKYGEVWEAKDQEDRRDFLRTYGVKVFVWGEGADKKDRGYAMNLGDIKTMAEELFPNRDRARFKLVHTHNAPEGYLSKIGIAVGLLKYGHPLEVKLRSPENS

SEQ ID NO: 1213 - protein, 605 aa, synthetic construct
MPESPPRALIVIRLSKVTDATTSPERQLAECRAICEKRGYEVVGVAEDLDVSGAVDPFDRKKRPHLARWLHGEHLDDNGEPVPFEVIVVYRVDRLTRSVRHLQKLVAWADDHKKLVVSATEAHFDTSLPFSAVLIALIGTVAEMELAGISERNASAARHNIQAGRYRGSTPPWGYVPSNDTGEWRLVRDKEQADVINEVARRVIEGEPLQKIAHDLTRRGILTPKDNFAKQRGREIKGREWSVTQLKRSLLSEAMLGHAVSGGAAVRNDDGSPVVRSEPILSREIYDRVAAELSSRAKRGEPNKRTSSLLLGVISCGNPCLHKQQGHECPEGCSGTCDEPVYKFNGGSHSQFPRYRCRTMTRAYKCGNRTIRADQADAQVERTILALLGSSERLERVWDAGEDHSAELADINDELVDLTSQLGVGAFRAGTPQRAKLDARIASLAERQAQLSSEAVVPAGWKLLPTGELFGDWWSRQDLTARNVWLRSMGVRARFKRDDKTLYIDLGNLNDLISGLKPGGTAQRVRGGLQAMERNGIQGMVFSDADSEVMPAPAAGYMWIQPVEGVWVYTSEALLAAAAERQALRREKIEDEFAYGPGDFDEDWD

SEQ ID NO: 1214 - protein, 523 aa, synthetic construct
MRQVITYLRFSSKPQERGDSIRRQKGLFERWLKDNPDAKVVDEFSDEGASAYHGHHLKGDFGRMLQNIQDGKYLSGQTVLLVESETRLNRQKARNTENLVDLITGKGVDVICLESGKIYTSTNIDDLDTSIQLKIAAHIAHQQSKEKSIKVSAAWEHRAQLALEGKQQLTKNVPGWIDPDTRKLNEHSSTVVTIFDLLLSGESLHNIARYLQANNIKSFSRREKANGFSVHSVRTILRSESTVGTLAASKRNDRPAIPNYYEPAIDVATFNKAQEILSKNRVGRAPASDNPITINLFKGIIRCQCGASVHPTGVKATYQGVYRCNNVPDGRCNVPTIKRKPFDKWMLDNIVGFLERDDGNNTDKRKAEIEYQISLVTSKLKKATTLLLELDDVTELKEQVKELNIQRSNLQSELDELNQRETLSDKPLHHLSEIDLTTKAGRVEAQLILSKFVQSIELQREMIIITLRNGTVIGKSRDLSPVLSQDLMKQVVSSPSPTDIDMFSVITSDEEFRKSGKQVTKRS

SEQ ID NO: 1215 - protein, 525 aa, synthetic construct
MPSAFSYVRFSSGKQAKGSSEHRQRAMLGQWLEQHPSFTLSDLRFEDLGRSGFSGEHLDHGLGQLLAAIDSGAIKSGDVILVEAVDRIGRLEPLEMLPLFSRIVKAGVSVITLEDGHVYDRSSVNETSLFLLVAKIQQAHEYSNRLSRRINASYTARREKAKAGLGIKRETPVWLTTDGQLVPHVAPHIAQAFQDYADGLGERRICRKLRESGLEEFSKTNATTVRRWLKNRTAIGYWNDIPDVYPHVVDPALFYQVQQRLDAPKVDRAKPSAHYLTGLVKCAVCGRNYNYKQRKHTDPAMLCTSRARLAGEGCSNSKTYPVIVLDQVRKLTSLPFLQHAMESASSQADPSSQRLAVIDGEIGELSRKISEATKALLVLGFTPEIQESLEQLKTAREALEEERATLLLPQAEKLTTAQLEAFSNGLLDDEPMKLNHVLQTAGYSMVVHPDGSIDVDGKRFVYEGASRKEKVYKLRLIGEDKQWSLPILTPQMATYKSLFMAAVRLPGDPSEEELRRFEEAKHSER

SEQ ID NO: 1216 - protein, 515 aa, synthetic construct
MSRPTGLTIDIYLRKSRKDLEEEKKASESGETYDTLERHRRTLFAVAKKERHNIANIYEEVVSGESVSERPQIQAMLRNLETSHIEGVLVMDLDRLGRGDMLDQGLLDRAFRYSGAKILTPTEVYDPESETWELVFGIKSLVAREELKAITRRMQRGRVASAGEGKSISKVPPYGYLRDENLRLYPDPETAWVVKKMFEMMRDGHGRIAVAQELDKLGIKPPNDKRRSWAPSSITAIIKNEVYLGTIIWGQVKYSKRNGKYKKTKLPRSKWTIKENAHEPIVSRELFEAANRAHTGRWRPSTNATKTLSNPLAGVLKCDVCGFTMLYQPRPNRPNDFIRCTQPTCQAVQKGATLALVEQHILDGLKQFAQELELQTEVPELDNDKDIAVKKSLVGNKQEEIAQLETQKSKLHDLLERGIYDVDTFLERQQNLNNRINGLQDDIRNIESEIKKEEVRNSSVLNLLPQLQTVISEYENADTESKNRLLKSVLEKATYLRKKEWTKRDQFIIQLYPKI

SEQ ID NO: 1217 - protein, 528 aa, synthetic construct
MYPYDVPDYAGSYRPESLDVCIYLRKSRKDVEEERRAIEEGSSYNALERHRKRLFAIAKAENHNIIDIFEEVASGESIQERPQMQQLLRKLEGNEIDGVLVIDLDRLGRGDMLDAGMIDRAFRYSSTKIITPTDVYDPDDESWELVFGIKSLISRQELKSITKRLQNGRIDSVKEGKHIGKKPPYGYLKDENLRLYPDPEKAWIVKKIFELMCDGKGRQMIAAELDRLGIDPPVTKRGAWDSSTITSIIKNEVYTGVIVWGKFKHKKRNGKYTRHKNPQEKWIMYENAHEPIISKELFDAANEAHSSRHKPAVITSKKLTNPLAGILKCKLCGYTMLIQTRKDRPHNYLRCNNPACKGKQKQSVFNLVEEKLLYSLQQIVDEYQAQKVEEVEIDDSKLISFKEKAIISKEKELKELQAQKGNLHDLLEQGIYTVEIFLERQKNLVERITSIENDIEVLQKEIETEQIKEHNKTEFIPALKTVIESYHKTTNIELKNQLLKTILSTVTYYRHPDWKTNEFEIQVYFKIS

SEQ ID NO: 1218 - protein, 486 aa, synthetic construct
MYPYDVPDYAGSAVGIYIRVSTQEQASEGHSIESQKKKLASYCEIQGWDDYRFYIEEGISGKNTNRPKLKLLMEHIEKGKINILLVYRLDRLTRSVIDLHKLLNFLQEHGCAFKSATETYDTTTANGRMSMGIVSLLAQWETENMSERIKLNLEHKVLVEGERVGAIPYGFDLSDDEKLVKNEKSAILLDMVERVENGWSVNRIVNYLNLTNNDRNWSPNGVLRLLRNPALYGATRWNDKIAENTHEGIISKERFNRLQQILADRSIHHRRDVKGTYIFQGVLRCPVCDQTLSVNRFIKKRKDGTEYCGVLYRCQPCIKQNKYNLAIGEARFLKALNEYMSTVEFQTVEDEVIPKKSEREMLESQLQQIARKREKYQKAWASDLMSDDEFEKLMVETRETYDECKQKLESCEDPIKIDETYLKEIVYMFHQTFNDLESEKQKEFISKFIRTIRYTVKEQQPIRPDKSKTGKGKQKVIITEVEFYQS

SEQ ID NO: 1219 - protein, 482 aa, synthetic construct
MNEKNLEIGAAYIRVSTDDQTELSPDAQLRVILEAAKKDGIIIPQEFVFMEDRGRSGRRADNRPEFQRMISTARQNPSPFRYLYLWKFSRFARNQEESAFYKGILRKKCGVTIKSVSEPIMEGMFGRLVEMIIEWSDEFYSVNLSGEVLRGMTQKALEHGYQLTPCLGYDAVGHGRPYVINEEQYQIVEFIHRSFFDGKDMTWIAREANRRGYHTRRGNPFDTRAVRIILTNSFYVGLVKWNDVTFQGTHECRESVTSVFSANQERLNRIHRPRGRRQASSCKHWLSGLLKCSICGASLGYNQTKDLTKRGHAFQCWKYTKGIHPGSCSVSSLKAEAAVLESLQMILETGEVEYTYEQREKHLDDNKLTLIQKSLERLDTKELRIREAYESGIDTLDEFKTNKARLQRERDQLMEELEELHSQEEPEDVPGKEILIERIQNVYDLLQSPDVDNDDKGNAVRSIIKKIVYIKESKTFCFYYYV

SEQ ID NO: 1220 - protein, 493 aa, synthetic construct
MKEKVSERKTGAIYIRVSTDKQEELSPDAQLRLLLDYAKKDSIDVPKEYIFQDNGISGRKANKRPAFQNMIALAKSKEHPIDTIIVWKFSRFARNQEESIVYKSLLKKNNVDVVSVSEPLIDGPFGSLIERIIEWMDEYYSIRLSGEVMRGMTQNAMRGHYQSDAPIGYTSPGDKKPPVINPDTVQIPLMIKDMFLSGSTQLQIARKLNDSGYRTKRGNLWDARGVRYVLENPFYIGKSRWNYTERGRRLKPADEVIYADGNWEALWDEDTFKEIQKRLALNMRKSKSRDISAAKHWLSGLLICSSCGGTLAFGGAHNMRGFQCWKYSKGFCSESHYISTGPIEKMVLEYLEAVMHSPALSYTVISSSSVDASSKLSDLERQLQKIDAKEKRIKAAYLNEIDTLEEYKANKTALEEERRTVEKEIEELTLSDVKYSKEDLDKKMKQNISDLLRVLRDESADYIQKGNMMRNVVDHIVFNRKNTSLDVFLKLVV

SEQ ID NO: 1221 - RNA, 20 nt, synthetic construct
  source 1..20: mol_type = other RNA
aagtgtgatcacttgggtgg

SEQ ID NO: 1222 - RNA, 11 nt, synthetic construct
  source 1..11: mol_type = other RNA
cccaagtgatc

SEQ ID NO: 1223 - RNA, 20 nt, synthetic construct
  source 1..20: mol_type = other RNA
gtatggaaaatgagagctgc

SEQ ID NO: 1224 - skipped

SEQ ID NO: 1225 - RNA, 20 nt, synthetic construct
  source 1..20: mol_type = other RNA
gacgtcacggcgctgcccca

SEQ ID NO: 1226 - skipped

SEQ ID NO: 1227 - RNA, 20 nt, synthetic construct
  source 1..20: mol_type = other RNA
gcgactcctggaagtggcca

SEQ ID NO: 1228 - RNA, 11 nt, synthetic construct
  source 1..11: mol_type = other RNA
ccacttccagg

SEQ ID NO: 1229 - RNA, 20 nt, synthetic construct
  source 1..20: mol_type = other RNA
gatgtctgcaggccagatga

SEQ ID NO: 1230 - RNA, 22 nt, synthetic construct
  source 1..22: mol_type = other RNA
aatgtgccatctggagcactca

SEQ ID NO: 1231 - RNA, 13 nt, synthetic construct
  source 1..13: mol_type = other RNA
tctggcctgcaga

SEQ ID NO: 1232 - RNA, 21 nt, synthetic construct
  source 1..21: mol_type = other RNA
gatgtacagagagcccagggc

SEQ ID NO: 1233 - DNA, 57 bp, synthetic construct
  source 1..57: mol_type = other DNA
acactctttccctacacgacgctcttccgatctnnnnatgagaccgtgacttgccag

SEQ ID NO: 1234 - DNA, 49 bp, synthetic construct
  source 1..49: mol_type = other DNA
tggagttcagacgtgtgctcttccgatcttgctgttgttgaaggcgttt

SEQ ID NO: 1235 - DNA, 58 bp, synthetic construct
  source 1..58: mol_type = other DNA
acactctttccctacacgacgctcttccgatctnnnntgggcacgcgtttaatataag

SEQ ID NO: 1236 - DNA, 49 bp, synthetic construct
  source 1..49: mol_type = other DNA
tggagttcagacgtgtgctcttccgatctagatccagccctggactagc

SEQ ID NO: 1237 - DNA, 18 bp, synthetic construct
  source 1..18: mol_type = other DNA
cttgccagccccacagag

SEQ ID NO: 1238 - DNA, 20 bp, synthetic construct
  source 1..20: mol_type = other DNA
ctgtgggacaagaggatcag

SEQ ID NO: 1239 - DNA, 19 bp, synthetic construct
  source 1..19: mol_type = other DNA
atgatcctgacgacggaga

SEQ ID NO: 1240 - DNA, 20 bp, synthetic construct
  source 1..20: mol_type = other DNA
tgtaccgtacaccactgaga

SEQ ID NO: 1241 - DNA, 27 bp, synthetic construct
  source 1..27: mol_type = other DNA
ttgtccatcactggcatctggactcca

SEQ ID NO: 1242 - DNA, 21 bp, synthetic construct
  source 1..21: mol_type = other DNA
ctctttatggcccttgtcact

SEQ ID NO: 1243 - DNA, 20 bp, synthetic construct
  source 1..20: mol_type = other DNA
ccatcacacatagaccctgc

SEQ ID NO: 1244 - DNA, 27 bp, synthetic construct
  source 1..27: mol_type = other DNA
cattggactctagaatgaagccaggca

SEQ ID NO: 1245 - DNA, 20 bp, synthetic construct
  source 1..20: mol_type = other DNA
cccgatattcctcaggtact

SEQ ID NO: 1246 - DNA, 21 bp, synthetic construct
  source 1..21: mol_type = other DNA
ggatgaaacccagacacatag

SEQ ID NO: 1247 - DNA, 24 bp, synthetic construct
  source 1..24: mol_type = other DNA
cgtcatccagcagagaatggaaag

SEQ ID NO: 1248 - protein, 16 aa, synthetic construct
RQIKIWFQNRRMKWKK

SEQ ID NO: 1249 - protein, 9 aa, synthetic construct
RRRRRRRRR

SEQ ID NO: 1250 - protein, 8 aa, synthetic construct
RRRRRRRR

SEQ ID NO: 1251 - RNA, 133 nt, synthetic construct
  source 1..133: mol_type = other RNA
gctggtacacggcagggtcagttttagagctagaaatagcaagttaaaataaggctagtccgttatcaacttgaaaaagtggcaccgagtcggtgcacgacggcggtctccgtcgtcaggatcatccctgccg

(19) United States
(12) Patent Application Publication
(10) Pub. No.: US 2026/0317757 A1
(43) Pub. Date: Oct. 15, 2026

(54) GENOME EDITING COMPOSITIONS AND METHODS OF USE

(71) Applicant: Prime Medicine, Inc., Cambridge, MA (US)

(72) Inventors: Christopher J. Podracky, Cambridge, MA (US); Emily Pomeroy, Cambridge, MA (US); Noah B. Bloch, Cambridge, MA (US); Andrew V. Anzalone, Cambridge, MA (US)

(21) Appl. No.: 19/145,260

(22) PCT Filed: Jan. 5, 2024
(86) PCT No.: PCT/US2024/010561

§ 371 Date: Jul. 2, 2025

Related U.S. Application Data
(60) Provisional application No. 63/478,654, filed on Jan. 5, 2023.
(60) Provisional application No. 63/603,479, filed on Nov. 28, 2023.

(51) Int. Cl. C12N 15/11; C07K 16/28; C12N 5/0783; C12N 9/12; C12N 9/22; C12N 15/88; C12N 15/90

(52) U.S. Cl. CPC C12N 15/11; C07K 16/2803; C12N 5/0636; C12N 9/1276; C12N 9/226; C12N 15/88; C12N 15/907; C12Y 207/07049; C12N 2310/20; C12N 2510/00

(57) ABSTRACT

Provided herein are compositions comprising prime editing systems, and methods of using prime editing systems to edit TRAC gene. Provided herein are compositions comprising edited cells, and methods to generate the edited cells. Also provided are methods of using the edited cells.

### CROSS REFERENCE

This application is a § 371 national-stage application based on PCT/US2024/010561, filed Jan. 5, 2024, which claims the benefit of U.S. Provisional Application No. 63/478,654, filed Jan. 5, 2023, and U.S. Provisional Application No. 63/603,479, filed Nov. 28, 2023, each of which is incorporated herein by reference in its entirety.

### REFERENCE TO A SEQUENCE LISTING XML

This application contains a Sequence Listing which has been submitted electronically in XML format. The Sequence Listing XML is incorporated herein by reference. Said XML file, created on Jun. 2, 2026, is named PMB-01701_SL.xml and is 864,702 bytes in size.

### BACKGROUND

Adoptive T cell therapy is an emerging cancer treatment modality. Adoptive T cell therapy can involve the ex vivo manipulation of T cells to express T-cell receptors (TCRs) or chimeric antigen receptors (CARs) engineered to recognize a tumor specific antigen. T cell therapy can involve autologous (i.e. patient-derived) T cells, which can avoid issues of immunogenicity and intolerance upon reintroduction of the ex vivo manipulated cells. However, manufacturing challenges (e.g., time, expense, poor quality/quantity) for autologous T cell therapies make allogeneic (i.e., donor-derived) T cell immunotherapies an attractive alternative. An obstacle to allogeneic cell therapies is that the endogenous TCR present on the infused allogeneic T cells may recognize non-tumor antigens in the recipient, leading to graft-versus-host disease (GvHD). The TCR contains a TCRα chain, encoded by a single T Cell Receptor Alpha Constant (TRAC) gene, complexed with a TCRβ chain, encoded by two T Cell Receptor Beta Constant (TRBC) genes. The TRAC gene is located in the human genome at 14q11.2. TRAC mRNA is approximately 1.5 kb. Disruption of the endogenous TCR can be achieved by eliminating expression of the TCRα chain because the TCRαβ dimer is necessary for full function of TCR. Therefore, the alloreactive potential of donor T cells to elicit GvHD is expected to be reduced or eliminated by genetically modifying the TRAC gene to reduce or eliminate its expression.

Engineered cellular receptors such as CARs can be used to redirect T cells to mediate tumor rejection. CARs can be transduced into T cells to generate CAR-T cells using retroviral, lentiviral, or other integrating vectors. However, because viral-mediated transduction results in random integration in the genome, application of such CAR-T cells may be limited by risks such as variegated expression, transcriptional silencing, or even oncogenic transformation.

Recent advances in genome editing enables target specific engineering in human genomes. For example, programmable nucleases such as CRISPR-Cas9 make double-strand DNA breaks (DSBs) that can disrupt genes by inducing mixtures of insertions and deletions (indels) at specific target sites. DSBs, however, are associated with undesired outcomes, including complex mixtures of products and translocations. There is a need in the art for compositions and methods for targeted delivery of transgenes and precise disruption of the TRAC gene without introducing DSBs.

### SUMMARY

In one aspect, provided herein is a prime editing composition or system comprising (A) a first prime editing guide RNA (PEgRNA) or one or more polynucleotides encoding the first PEgRNA and (B) a second PEgRNA or one or more polynucleotides encoding the second PEgRNA, wherein the first PEgRNA comprises: (i) a first spacer that is complementary to a first search target sequence on a first strand of a TRAC gene, (ii) a first gRNA core capable of binding to a Cas9 protein; and (iii) a first extension arm comprising a first editing template and a first primer binding site (PBS), wherein the first spacer comprises at its 3′ end nucleotides 4-20 of a sequence selected from the group consisting of SEQ ID NOs: 4, 61, 88, and 150 and wherein the first PBS comprises at its 5′ end a sequence that is the reverse complement of nucleotides 13-17 of the selected sequence; wherein the second PEgRNA comprises: (i) a second spacer that is complementary to a second search target sequence on a second strand of the TRAC gene complementary to the first strand, (ii) a second gRNA core capable of binding to a Cas9 protein; and (iii) a second extension arm comprising a second editing template and a second PBS, wherein the second spacer comprises at its 3′ end nucleotides 4-20 of a sequence selected from the group consisting of SEQ ID NOs: 177, 233, 260, 287, 314, 341, 368, 414, 441, 468, 495, 522, and 566, and wherein the second PBS comprises at its 5′ end a sequence that is the reverse complement of nucleotides 13-17 of the selected sequence; and wherein (a) the first editing template comprises a region of complementarity to the second editing template; (b) the first editing template comprises nucleotides 8-17 of the selected sequence for the second spacer, and the second editing template comprises nucleotides 8-17 of the selected sequence for the first spacer; or (c) the first editing template comprises nucleotides 8-17 of the selected sequence for the second spacer and a region of complementarity to the second editing template, and the second editing template comprises nucleotides 8-17 of the selected sequence for the first spacer and a region of complementarity to the first editing template.

In some embodiments, the selected sequence for the first spacer is SEQ ID NO: 4 or 88.

In some embodiments, the selected sequence for the first spacer is SEQ ID NO: 88.

In some embodiments, the selected sequence for the second spacer is SEQ ID NO: 177, 368, or 522.

In some embodiments, the selected sequence for the second spacer is SEQ ID NO: 177.

In some embodiments, the first spacer and/or the second spacer is from 16 to 22 nucleotides in length.

In some embodiments, the first spacer and/or the second spacer is 20 nucleotides in length and comprises the selected sequence.

In some embodiments, the first PBS is 8-17 nucleotides in length and comprises at its 5′ end a sequence that is the reverse complement of nucleotides 10-17, 9-17, 8-17, 7-17, 6-17, 5-17, 4-17, 3-17, 2-17, or 1-17 of the selected sequence for the first spacer.

In some embodiments, the first PBS is 8-13 nucleotides in length.

In some embodiments, the first PBS is 10, 11, or 12 nucleotides in length.

In some embodiments, the second PBS is 7-17 nucleotides in length and comprises at its 5′ end a sequence that is the reverse complement of nucleotides 11-17, 10-17, 9-17, 8-17, 7-17, 6-17, 5-17, 4-17, 3-17, 2-17, or 1-17 of the selected sequence for the second spacer.

In some embodiments, the second PBS is 8-13 nucleotides in length.

In some embodiments, the second PBS is 11, 12, or 13 nucleotides in length.

In some embodiments, the first gRNA core and the second gRNA core comprise the same sequence.

In some embodiments, the first gRNA core, the second gRNA core, or both comprise SEQ ID NO: 590.

In some embodiments, the first spacer, the first gRNA core, the first editing template, and the first PBS form a contiguous sequence in a single molecule.

In some embodiments, the first PEgRNA comprises from 5′ to 3′ the first spacer, the first gRNA core, the first editing template, and the first PBS.

In some embodiments, the second spacer, the second gRNA core, the second editing template, and the second PBS form a contiguous sequence in a single molecule.

In some embodiments, the second pegRNA comprises from 5′ to 3′ the second spacer, the second gRNA core, the second editing template, and the second PBS.

In some embodiments, the first editing template comprises a region of complementarity to the second editing template.

In some embodiments, the first editing template and the second editing template each encodes all or a fragment of a recombinase recognition sequence (RSS) or the reverse complement thereof, wherein the first editing template encodes at least a 5′ portion of the RSS or the reverse complement thereof, wherein the second editing template encodes at least a 3′ portion of the RSS or the reverse complement thereof, and wherein at least 10 nucleotides at the 5′ ends of the first and the second editing templates have perfect reverse complementarity to each other.

In some embodiments, at least 15, 20, 25, or 30 nucleotides at the 5′ ends of the first and the second editing templates have perfect reverse complementarity to each other, optionally wherein at least 20, 21, 22, 23, 24, 25, 26, or 27 nucleotides at the 5′ ends of the first and the second editing templates have prefect reverse complementarity to each other.

In some embodiments, the first editing template encodes the RSS.

In some embodiments, the second editing template encodes the RSS.

In some embodiments, the RSS is an attB sequence recognized by a Bxb1 recombinase.

In some embodiments, the RSS is an attP sequence recognized by a Bxb1 recombinase.

In some embodiments, the RSS is an attB sequence recognized by a Pa01 recombinase.

In some embodiments, the RSS is an attP sequence recognized by a Pa01 recombinase.

In some embodiments, the first editing template comprises an RTT #1 from Table 6 and the second editing template comprises an RTT #2 from the same RTT Pair in Table 6, or wherein the first editing template comprises an RTT #2 from Table 6 and the second editing template comprises an RTT #1 from the same RTT Pair in Table 6.

In some embodiments, the first editing template comprises SEQ ID NO: 23 and the second editing template comprises SEQ ID NO: 196.

In some embodiments, the first editing template comprises SEQ ID NO: 24 and the second editing template comprises SEQ ID NO: 106.

In some embodiments, the first editing template comprises SEQ ID NO: 27 and the second editing template comprises SEQ ID NO: 107.

In some embodiments, first editing template comprises SEQ ID NO: 106 and the second editing template comprises SEQ ID NO: 24.

In some embodiments, the first editing template comprises SEQ ID NO: 107 and the second editing template comprises SEQ ID NO: 27.

In some embodiments, the first editing template comprises SEQ ID NO: 25 and the second editing template comprises SEQ ID NO: 197.

In some embodiments, the first editing template comprises SEQ ID NO: 28 and the second editing template comprises SEQ ID NO: 199.

In some embodiments, the first editing template comprises SEQ ID NO: 22 and the second editing template comprises SEQ ID NO: 195.

In some embodiments, the first editing template comprises SEQ ID NO: 26 and the second editing template comprises SEQ ID NO: 198.

In some embodiments, the first editing template comprises a 5′ fragment of an RTT listed in Table 6 and wherein the second editing template comprises a full length or 5′ fragment of the corresponding RTT pair and wherein at least 10 nucleotides at the 5′ ends of the first and the second editing templates have perfect reverse complementarity to each other.

In some embodiments, the second editing template comprises a 5′ fragment of an RTT listed in Table 6 and wherein the first editing template comprises a full length or 5′ fragment of the corresponding RTT pair and wherein at least 10 nucleotides at the 5′ ends of the first and the second editing templates have perfect reverse complementarity to each other.

In some embodiments, at least 15, 20, 25, or 30 nucleotides at the 5′ ends of the first and the second editing templates have perfect reverse complementarity to each other, optionally wherein at least 20, 21, 22, 23, 24, 25, 26, or 27 nucleotides at the 5′ ends of the first and the second editing templates have prefect reverse complementarity to each other.

In some embodiments, the length of the region of complementarity of the first editing template is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% of the length of the first editing template, optionally wherein the length of the region of complementarity of the first editing template is at least 52%, at least 53%, or at least 55% of the length of the first editing template.

In some embodiments, the length of the region of complementarity of the second editing template is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% of the length of the second editing template, optionally wherein the length of the region of complementarity of the second editing template is at least 52%, at least 53%, or at least 55% of the length of the second editing template.

In some embodiments, (a) the first spacer comprises SEQ ID NO: 4, and the first PBS comprises SEQ ID NO: 13, or the first spacer comprises SEQ ID NO: 88, and the first PBS comprises SEQ ID NO: 96, or the first spacer comprises SEQ ID NO: 88, and the first PBS comprises SEQ ID NO: 157, or the first spacer comprises SEQ ID NO: 88, and the first PBS comprises SEQ ID NO: 99, or the first spacer comprises SEQ ID NO: 88, and the first PBS comprises SEQ ID NO: 97; and (b) the second spacer comprises SEQ ID NO: 177, and the second PBS comprises SEQ ID NO: 188, or the second spacer comprises SEQ ID NO: 368, and the second PBS comprises SEQ ID NO: 376.

In some embodiments, (a) the first spacer SEQ ID NO: 4, and the first PBS has the sequence according to SEQ ID NO: 14 or SEQ ID NO: 15; or the first spacer comprises SEQ ID NO: 88, and the first PBS has the sequence according to SEQ ID NO: 96 or SEQ ID NO: 98; and (b) the second spacer comprises SEQ ID NO: 177, and the second PBS has the sequence according to SEQ ID NO: 186 or SEQ ID NO: 188, the second spacer comprises SEQ ID NO: 368, and the second PBS has the sequence according to SEQ ID NO: 373 or SEQ ID NO: 374; or the second spacer comprises SEQ ID NO: 522, and the second PBS has the sequence according to SEQ ID NO: 531 or 533.

In some embodiments, the first spacer comprises SEQ ID NO: 88, and the first PBS has the sequence according to SEQ ID NO: 96, and wherein the second spacer comprises SEQ ID NO: 177, and the second PBS has the sequence according to SEQ ID NO: 186.

In some embodiments, the first editing template comprises SEQ ID NO: 27 and the second editing template comprises SEQ ID NO: 107.

In some embodiments, the first editing template comprises SEQ ID NO: 107 and the second editing template comprises SEQ ID NO: 27.

In some embodiments, the first PEgRNA comprises a sequence selected from the group consisting of SEQ ID NOs: 36, 118, 123, and 1132-1134; and wherein the second PEgRNA comprises a sequence selected from the group consisting of SEQ ID NOs: 1135-1142.

In some embodiments, the first PEgRNA comprises SEQ ID NO: 118 and the second PEgRNA comprises SEQ ID NO: 1140.

In some embodiments, the first PERNA comprises SEQ ID NO: 136 and the second PEgRNA comprises SEQ ID NO: 224.

In some embodiments, the first PEgRNA comprises SEQ ID NO: 51 and the second PERNA In some embodiments, the first PEgRNA comprises SEQ ID NO: 126 and the second PERNA comprises SEQ ID NO: 220.

In some embodiments, the first PEgRNA comprises SEQ ID NO: 44 and the second PEgRNA comprises SEQ ID NO: 550.

In some embodiments, the first PEgRNA comprises SEQ ID NO: 111 and the second PEgRNA comprises SEQ ID NO: 1251.

In some embodiments, the first PEgRNA comprises a sequence selected from the group consisting of SEQ ID NOs: 30, 31, 32, 33, 34, 36, 38, 39, 43, 44, 49, 50, 51, 52, 53, 79, 80, 82, 109, 111, 112, 114, 115, 118, 120, 122, 123, 125, 126, 129, 130, 134, 135, 136, 140, 141, 143, 168, 169, 171, 592, 593, 594, and 1132; and wherein the second PEgRNA comprises a sequence selected from the group consisting of SEQ ID NOs: 203, 207, 210, 211, 212, 215, 217, 219, 220, 221, 222, 224, 226, 228, 251, 252, 254, 278, 279, 281, 305, 306, 308, 332, 333, 335, 359, 360, 362, 388, 390, 392, 395, 398, 397, 400, 401, 403, 404, 405, 408, 410, 432, 433, 435, 459, 460, 462, 486, 487, 489, 513, 514, 516, 541, 542, 543, 545, 546, 547, 549, 550, 552, 554, 555, 556, 558, 561, 562, 584, 585, 587, 591, 595, 597, 599, 601, 1127, 1128, 1135, 1136, 1137, 1138, 1139, 1140, and 1141.

In some embodiments, the first PEgRNA comprises a sequence selected from the group consisting of SEQ ID NOs: 30, 44, 109, and 126; and wherein the second PERNA comprises a sequence selected from the group consisting of SEQ ID NOs: 207, 221, 388, and 400.

In some embodiments, the first PEgRNA comprises a sequence selected from the group consisting of SEQ ID NOs: 136, 141, 51, and 53; and wherein the second PEgRNA comprises a sequence selected from the group consisting of SEQ ID NOs: 226, 403, 558, 556, and 224.

In some embodiments, the first PEgRNA comprises SEQ ID NO: 136, and wherein the second PEgRNA comprises SEQ ID NO: 224.

In some embodiments, the first PEgRNA and/or the second PEgRNA further comprises a 3′ motif, optionally wherein the 3′ motif is connected to the 3′ end of the first PBS or the second PBS via a linker.

In some embodiments, the first PEgRNA and/or the second PEgRNA further comprises 5′mN*mN*mN* and 3′ mN*mN*mN*N modifications, where m indicates that the nucleotide contains a 2′-O-Me modification and a * indicates the presence of a phosphorothioate bond.

In some embodiments, the prime editing composition or system of any one of the embodiments herein further comprises a prime editor or one or more polynucleotides encoding the prime editor, wherein the prime editor comprises (a) a Cas9 nickase having a nuclease inactivating mutation in a HNH domain and (b) a reverse transcriptase.

In some embodiments, the Cas9 nickase comprises an amino acid sequence having at least 80%, 85%, 90%, 95%, 96%, 97%, 98%, 99%, or 100% identity to SEQ ID NO: 1007.

In some embodiments, the reverse transcriptase comprises an amino acid sequence having at least 80%, 85%, 90%, 95%, 96%, 97%, 98%, 99%, or 100% identity to SEQ ID NO: 1003.

In some embodiments, the prime editor is a fusion protein.

In some embodiments, the fusion protein comprises SEQ ID NO: 1033.

In some embodiments, the one or more polynucleotides encoding the prime editor comprise (a) a first sequence encoding an N-terminal portion of the Cas9 nickase and an intein-N and (b) a second sequence encoding an intein-C, a C-terminal portion of the Cas9 nickase, and the reverse transcriptase.

In some embodiments, the prime editing composition or system of any one of the embodiments herein further comprises a recombinase that recognizes the one or more recombinase recognition sequences (RSSs) or one or more polynucleotides encoding the recombinase.

In some embodiments, the recombinase is Bxb1 or Pa01.

In some embodiments, the recombinase is a Bxb1 comprising an amino acid sequence having at least 80%, 85%, 90%, 95%, 96%, 97%, 98%, 99%, or 100% identity to SEQ ID NO: 1131.

In some embodiments, the sequence identities are determined by Needleman-Wunsch alignment of two protein sequences with Gap Costs set to Existence: 11 Extension: 1 where percent identity is calculated by dividing the number of identities by the length of the alignment.

In some embodiments, the recombinase is fused or linked to the prime editor.

The prime editing composition or system of any one of the embodiments herein further comprises a DNA polynucleotide that comprises (a) a donor sequence and (b) a second RSS recognized by the recombinase.

In some embodiments, (i) the RSS comprises a Bxb1 attB sequence provided in Table 5, and the second RSS comprises a corresponding attP sequence provided in Table 5, or (ii) the RSS comprises a Bxb1 attP sequence provided in Table 5, and the second RSS comprises a corresponding attB sequence provided in Table 5.

In some embodiments, the RSS sequence comprises SEQ ID NO: 1187, and the second RSS comprises SEQ ID NO: 1188.

In some embodiments, the donor sequence comprises an open reading frame that encodes a polypeptide.

In some embodiments, the donor sequence encodes a chimeric antigen receptor (CAR).

In some embodiments, the donor sequence encodes a CD19 CAR.

In some embodiments, the donor sequence comprises a splice acceptor sequence.

In some embodiments, the prime editing composition or system of any one of the embodiments herein comprises one or more vectors that comprises the one or more polynucleotides encoding the first PEgRNA, the one or more polynucleotides encoding the second PEgRNA, and the one or more polynucleotides encoding the prime editor.

In some embodiments, the prime editing composition or system of any one of the embodiments herein comprises one or more vectors that comprises the one or more polynucleotides encoding the first PEgRNA, the one or more polynucleotides encoding the second PEgRNA, the one or more polynucleotides encoding the prime editor, the one or more polynucleotides encoding the recombinase, and the donor sequence.

In some embodiments, the one or more vectors are AAV vectors.

In some embodiments, the one or more polynucleotides encoding the prime editor and/or the one or more polynucleotides encoding the recombinase are mRNA.

In some embodiments, the prime editing composition or system of any one of the embodiments herein further comprises a third PERNA or a nucleic acid encoding the third PERNA, wherein the third PEgRNA comprises: (i) a third spacer that is complementary to a search target sequence on a first strand of a β2-microglobulin (B2M) gene; (ii) a third gRNA core capable of binding to a Cas9 protein; and (iii) a third extension arm comprising: (a) a third editing template that comprises a region of complementarity to an editing target sequence on a second strand of the B2M gene, and (b) a third primer binding site (PBS) that comprises a sequence that is a reverse complement of a portion of the third spacer, wherein the first and second strand of the B2M gene are complementary to each other, and wherein the third editing template encodes one or more nucleotide changes compared to the editing target sequence.

In some embodiments, the third spacer comprises at its 3′ end SEQ ID NO: 1063.

In some embodiments, the third PBS comprises at its 5′ end a sequence that is a reverse complement of nucleotides 10-14 of SEQ ID NO: 1063.

In some embodiments, the editing template encodes one or more in-frame stop codons or the complement thereof in the B2M gene.

In some embodiments, the one or more in-frame stop codons comprise a nonsense mutation in the B2M gene.

In some embodiments, the one or more in-frame stop codons comprise an insertion in the B2M gene.

In some embodiments, the editing template encodes an insertion of two in-frame stop codons in the B2M gene.

In some embodiments, the insertion is TAATAA or TTATTA.

In some embodiments, the editing template encodes a frameshift mutation in the B2M gene.

In some embodiments, the frameshift mutation is an insertion.

In some embodiments, the insertion is c.50insG or the complement thereof.

In some embodiments, the frameshift mutation is a deletion.

In some embodiments, the deletion is c.51delC or the complement thereof.

In some embodiments, the prime editing composition or system further comprises a third PEgRNA or a nucleic acid encoding the third PEgRNA, wherein the third PEgRNA comprises: a. a third spacer comprising at its 3′ end SEQ ID NO: 1063; b. a third gRNA core capable of binding to a Cas9 protein; and c. a third extension arm comprising: i. a third editing template comprising at its 3′ end: (A) nucleotides 13-24 of SEQ ID NO: 1079, (B) nucleotides 12-20 of SEQ ID NO: 1085, or (C) nucleotides 7-17 of SEQ ID NO: 1089, and ii. a third primer binding site (PBS) comprising at its 5′ end a sequence that is a reverse complement of nucleotides 10-14 of SEQ ID NO: 1063.

In some embodiments, the third spacer is from 17-22 nucleotides in length.

In some embodiments, the third spacer comprises at its 3′ end any one of SEQ ID NOs: 1060-1063.

In some embodiments, the third editing template comprises at its 3′ end nucleotides 13-24 of SEQ ID NO: 1077.

In some embodiments, the third editing template comprises at its 3′ end SEQ ID NO: 1077.

In some embodiments, the third editing template comprises at its 3′ end any one of SEQ ID NOs: 1078 or 1079.

In some embodiments, the third editing template comprises at its 3′ end nucleotides 12-20 of SEQ ID NO: 1085.

In some embodiments, the third editing template comprises at its 3′ end SEQ ID NO: 1081.

In some embodiments, the third editing template comprises at its 3′ end any one of SEQ ID Nos: 1080, 1082-1085.

In some embodiments, the third editing template comprises at its 3′ end nucleotides 7-17 of SEQ ID NO: 1089.

In some embodiments, the third editing template comprises at its 3′ end any one of SEQ ID NOs: 1087-1089.

In some embodiments, the third editing template has a length of 24 nucleotides or less.

In some embodiments, the third editing template has a length of 20 nucleotides or less.

In some embodiments, the third editing template has a length of 10 to 20 nucleotides.

In some embodiments, the third editing template has a length of 12 to 20 nucleotides.

In some embodiments, third editing template has a length of 11 to 17 nucleotides.

In some embodiments, the third editing template is 16 to 24 nucleotides in length.

In some embodiments, the third PBS comprises at its 3′ end a sequence set forth in any one of sequence numbers 1064-1076.

In some embodiments, the third PBS has a length of 17 nucleotides or less.

In some embodiments, the third PBS is 8 to 15 nucleotides in length.

In some embodiments, the third PBS is 8 to 14 nucleotides in length.

In some embodiments, the third PBS is 12 nucleotides in length.

In some embodiments, the third spacer, the third gRNA core, the third editing template, and the third PBS form a contiguous sequence in a single molecule.

In some embodiments, the single molecule comprises from 5′ to 3′, the third spacer, the third gRNA core, the third editing template and the third PBS.

In some embodiments, the third PEgRNA comprises a sequence selected from any one SEQ ID NOs: 1090-1120.

In some embodiments, the prime editing composition or system of any one of the embodiments herein further comprises a nick guide RNA (ngRNA), or a nucleic acid encoding the ngRNA, wherein the ngRNA comprises: (i) a ngRNA spacer that is complementary to a ngRNA target sequence on the second strand of the B2M gene; and (ii) an ngRNA core capable of binding a Cas9 protein.

In some embodiments, the ngRNA spacer comprises at its 3′ end a sequence corresponding to nucleotides 4-20, 3-20, 2-20, or 1-20 of any one of SEQ ID NOs: 1121-1126.

In some embodiments, ngRNA spacer comprises at its 3′ end any one of SEQ ID NOs: 1121-1126.

In some embodiments, the ngRNA spacer comprises at its 3′ end SEQ ID NO: 1126.

In some embodiments, the one or more nucleotide encoded by the editing template is c.51delC or the complement thereof, and wherein the ngRNA spacer comprises at its 3′ end sequence corresponding to nucleotides 4-20, 3-20, 2-20, or 1-20 of SEQ ID NO: 1124.

In some embodiments, the one or more nucleotide encoded by the editing template is c.50insG or the complement thereof, and wherein the ngRNA spacer comprises at its 3′ end sequence corresponding to nucleotides 4-20, 3-20, 2-20, or 1-20 of SEQ ID NO: 1125 or 1126.

In some embodiments, the ngRNA comprises SEQ ID NO: 1129.

In one aspect, provided herein is an LNP comprising the prime editing composition or system of any one of the embodiments herein.

In one aspect, provided herein is a pharmaceutical composition comprising the prime editing composition or system of any one of the embodiments herein or the LNP of any one of the embodiments herein and a pharmaceutically acceptable excipient.

In one aspect, provided herein is a method of editing a TRAC gene, the method comprising contacting the TRAC gene with (a) the prime editing composition or system of any one of the embodiments herein and a prime editor comprising a Cas9 nickase having a nuclease inactivation mutation in a HNH domain and a reverse transcriptase or (b) the prime editing composition or system of any one of the embodiments herein.

In some embodiments, the method further comprises contacting the TRAC gene with a recombinase or one or more polynucleotides encoding the recombinase and a DNA polynucleotide comprising (a) a donor sequence and (b) one or more recombinase recognition sequences recognized by the recombinase.

In one aspect, provided herein is a method of inserting a donor sequence into a TRAC gene, the method comprises contacting the TRAC gene with the prime editing composition or system of any one of the embodiments herein or the LNP of any one of the embodiments herein.

In one aspect, provided herein is a method of making a modified cell, the method comprising contacting a cell with the prime editing composition or system of any one of the embodiments herein or the LNP of any one of the embodiments herein.

In some embodiments, the TRAC gene is in a cell.

In some embodiments, the cell is a mammalian cell.

In some embodiments, the cell is a human cell.

In some embodiments, the cell is an immune cell, optionally wherein, the cell is a T cell.

In some embodiments, the cell is in a subject.

In some embodiments, the cell is from a subject.

In some embodiments, the subject is a human.

In some embodiments, the method of any one of the embodiments herein further comprises editing a B2M gene.

In some embodiments, the editing comprises contacting the B2M gene with the prime editing composition or system of any one of the embodiments herein and a prime editor comprising a Cas9 nickase having a nuclease inactivation mutation in a HNH domain and a reverse transcriptase.

In one aspect, provided herein is a cell generated by the method of any one of the embodiments herein.

In one aspect, provided herein is an edited TRAC gene that comprises GGCTTGTCGACGACGGCGGTCTCAGTGGTGTACGGTACAAACC (SEQ ID NO: 1046) and/or GGTTTGTCTGGTCAACCACCGCGGTCTCCGTCGTCAGGATCAT (SEQ ID NO: 1047) relative to a wildtype TRAC gene.

In some embodiments, the edited TRAC gene comprises an insert sequence comprising, from 5′ to 3′, GGCTTGTCGACGACGGCGGTCTCAGTGGTGTACGGTACAAACC (SEQ ID NO: 1046), a donor sequence, and GGTTTGTCTGGTCAACCACCGCGGTCTCCGTCGTCAGGATCAT (SEQ ID NO: 1047).

In some embodiments, the edited TRAC gene comprises an insert sequence comprising, from 5′ to 3′, GGTTTGTCTGGTCAACCACCGCGGTCTCCGTCGTCAGGATCAT (SEQ ID NO: 1047), a donor sequence, and GGCTTGTCGACGACGGCGGTCTCAGTGGTGTACGGTACAAACC (SEQ ID NO: 1046).

In some embodiments, the donor sequence encodes a chimeric antigen receptor (CAR), optionally wherein the donor encodes a CD19 CAR.

In some embodiments, the insert sequence is between a first chromosome location and a second chromosome location, wherein the first chromosome location is selected from the group consisting of human chromosome 14 positions 22547458, 22547457, 22547449, and 22547448, and wherein the second chromosome location is selected from the group consisting of human chromosome 14 positions 22547533, 22547523, 22547491, 22547528, 22547497, 22547579, 22547522, 22547485, 22547506, 22547560, 22547505, 22547529, and 22547490.

In some embodiments, the insert sequence is between human chromosome 14 positions 22547458 and 22547533.

In some embodiments, the insert sequence is between human chromosome 14 positions 22547458 and 22547522.

In some embodiments, the insert sequence is between human chromosome 14 positions 22547458 and 22547529.

In some embodiments, the insert sequence is between human chromosome 14 positions 22547449 and 22547533.

In some embodiments, the insert sequence is between human chromosome 14 positions 22547449 and 22547522.

In some embodiments, the insert sequence is between human chromosome 14 positions 22547449 and 22547529.

In some embodiments, the T cell further comprises premature stop codon relative to a wildtype B2M gene.

In some embodiments, the T cell further comprises a B2M gene comprising a c.51delC edit relative to a wildtype B2M gene.

In some embodiments, the T cell further comprises a B2M gene comprising a c.50insG edit relative to a wildtype B2M gene.

In some embodiments, the T cell further comprises a B2M gene comprising a c.54insTAATAA edit relative to a wildtype B2M gene.

In some embodiments, the human chromosome locations and coding sequence locations are as set forth in Genome Reference Consortium Human Build 38 (GrCh38).

In one aspect, provided herein is a prime editing composition or system comprising (a) a prime editing guide RNA (PEgRNA) or one or more polynucleotides encoding the PEgRNA, wherein the PEgRNA comprises: (i) a spacer that is complementary to a search target sequence on a first strand of a target gene, (ii) a gRNA core capable of binding to a Cas9 nickase, and (iii) an extension arm comprising a primer binding site (PBS) that comprises a region of complementarity to a second strand of the target gene and an editing template encoding a recombinase recognition sequence (RSS) recognized by a Pa01 recombinase; (b) a prime editor comprising the Cas9 nickase capable of nicking the second strand of the target gene at a nick site and a reverse transcriptase, or one or more polynucleotides encoding the prime editor; and (c) a Pa01 recombinase or one or more polynucleotides encoding the Pa01 recombinase.

In some embodiments, the PBS comprises a region of complementarity to a region upstream of the nick site.

In some embodiments, the editing template comprises a region of complementarity to a region downstream of the nick site.

In some embodiments, the Cas9 nickase comprises a nuclease inactivation mutation in a HNH domain.

In one aspect, provided herein is a prime editing composition or system comprising (A) a first prime editing guide RNA (PEgRNA) or one or more polynucleotides encoding the first PEgRNA, (B) a second PEgRNA or one or more polynucleotides encoding the second PEgRNA, (C) a prime editor comprising a Cas9 nickase having a nuclease inactivation mutation in a HNH domain and a reverse transcriptase, or one or more polynucleotides encoding the prime editor, and (D) a Pa01 recombinase or one or more polynucleotides encoding the Pa01 recombinase, wherein the first PEgRNA comprises: (i) a first spacer that is complementary to a first search target sequence on a first strand of a target gene, (ii) a first gRNA core capable of binding to the Cas9 nickase; and (iii) a first extension arm comprising a first editing template and a first primer binding site (PBS), wherein the second PEgRNA comprises: (i) a second spacer that is complementary to a second search target sequence on a second strand of the target gene complementary to the first strand, (ii) a second gRNA core capable of binding to the Cas9 nickase; and (iii) a second extension arm comprising a second editing template and a second PBS, wherein the first editing template comprises a region of complementarity to the second editing template, wherein the first editing template and the second editing template each encodes all or a fragment of a recombinase recognition sequence (RSS) or the reverse complement thereof, wherein the first editing template encodes at least a 5′ portion of the RSS or the reverse complement thereof, wherein the second editing template encodes at least a 3′ portion of the RSS or the reverse complement thereof, wherein at least 10 nucleotides at the 5′ end of the first and the second editing templates have perfect reverse complementarity to each other, and wherein the RSS is recognized by the Pa01 recombinase.

In some embodiments, at least 15, 20, 25, or 30 nucleotides at the 5′ end of the first and second editing templates have perfect reverse complementarity to each other.

In some embodiments, the first editing template encodes the RSS.

In some embodiments, the second editing template encodes the RSS.

In some embodiments, the first editing template comprises a 5′ fragment of an RTT listed in RTT pair 2 or 3 of Table 6 and wherein the second editing template comprises a full length or 5′ fragment of the corresponding RTT pair and wherein at least 10 nucleotides at the 5′ end of the first and second editing templates have perfect reverse complementarity to each other.

In some embodiments, the second editing template comprises a 5′ fragment of an RTT listed in RTT pair 2 or 3 of Table 6 and wherein the first editing template comprises a full length or 5′ fragment of the corresponding RTT pair and wherein at least 10 nucleotides at the 5′ end of the first and second editing templates have perfect reverse complementarity to each other.

In some embodiments, the first PBS comprises a region of complementarity to a region upstream of a nick site in the second strand, and wherein the second PBS comprises a region of complementarity to a region upstream of a nick site in the first strand.

In some embodiments, the prime editing composition or system of any one of the embodiments herein further comprises a DNA polynucleotide that comprises (i) a second RSS recognized by the Pa01 recombinase, and (ii) a donor sequence.

In some embodiments, the target gene is TRAC.

In one aspect, provided herein is a method for integrating a donor sequence in a target gene, the method comprising contacting the target gene with the prime editing composition or system of any one of the embodiments herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a schematic of a prime editing guide RNA (PEgRNA) binding to a double-stranded target DNA sequence.

FIG. 2 depicts a PEgRNA architectural overview in an exemplary schematic of PEgRNA designed for a prime editor.

FIG. 3 is a schematic showing the spacer and gRNA core part of an exemplary guide RNA, in two separate molecules. The rest of the PEgRNA structure is not shown.

FIG. 4A depicts an exemplary schematic of a dual prime editing system for editing both strands of a double-stranded target DNA. Same color/shading indicates complementarity or identity between sequences.

FIG. 4B depicts an exemplary schematic of dual prime editing with a replacement duplex (RD) comprising an overlap duplex (OD). Same color/shading indicates complementarity or identity between sequences.

FIG. 4C depicts an exemplary schematic of dual prime editing. Same color/shading indicates complementarity or identity between sequences.

FIG. 4D depicts an exemplary schematic of dual prime editing. Same color/shading indicates complementarity or identity between sequences.

FIG. 4E depicts an exemplary schematic of dual prime editing. Same color/shading indicates complementarity or identity between sequences.

FIG. 4F depicts an exemplary schematic of dual prime editing. Same color/shading indicates complementarity or identity between sequences.

FIG. 4G depicts an exemplary schematic of dual prime editing. Same color/shading indicates complementarity or identity between sequences.

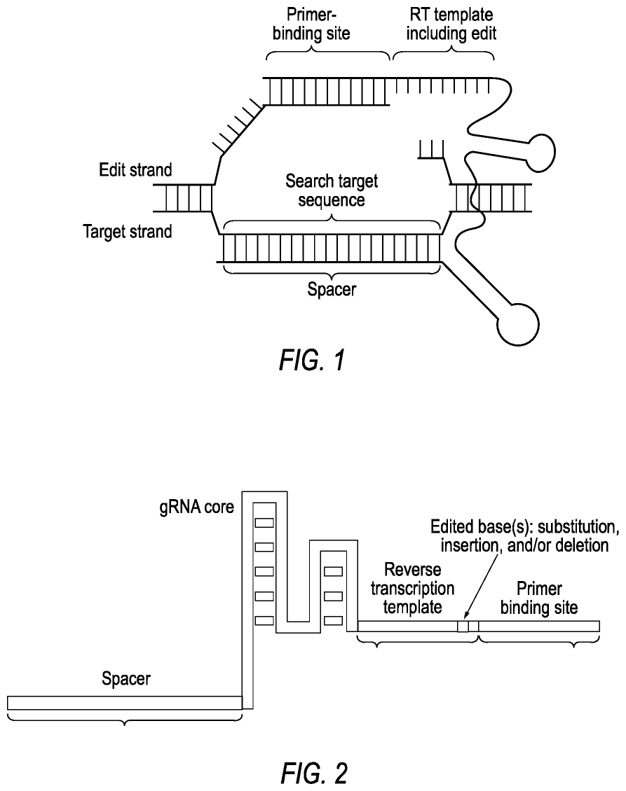

### DETAILED DESCRIPTION OF THE INVENTION

Provided herein, in some embodiments, are systems, compositions and methods to edit the target gene T-cell receptor a constant (TRAC) with dual prime editing. Compositions provided herein can comprise prime editors (PEs) that may use engineered guide polynucleotides, e.g., prime editing guide RNAs (PEgRNAs), that can direct PEs to specific DNA targets and can encode DNA edits on the target gene TRAC that serve a variety of functions, including disruption of the target gene (e.g., by introducing one or mutation in the target gene), introduction of exogenous sequences in to the TRAC gene (e.g., one or more recombinase recognition sequences), and integration of DNA donors in the gene (e.g., expression cassettes, ORFs). Also provided are compositions that comprise edited cells generated by the methods disclosed herein.

The following description and examples illustrate embodiments of the present disclosure in detail. It is to be understood that this disclosure is not limited to the particular embodiments described herein and as such can vary. Those of skill in the art will recognize that there are numerous variations and modifications of this disclosure, which are encompassed within its scope. Although various features of the present disclosure can be described in the context of a single embodiment, the features can also be provided separately or in any suitable combination. Conversely, although the present disclosure can be described herein in the context of separate embodiments for clarity, the present disclosure can also be implemented in a single embodiment.

### Definitions

Unless defined otherwise, all technical and scientific terms used herein have the same meaning as is commonly understood by one of ordinary skill in the art.

The terminology used herein is for the purpose of describing particular embodiments only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used herein, they mean “comprising”.

Unless otherwise specified, the words “comprising”, “comprise”, “comprises”, “having”, “have”, “has”, “including”, “includes”, “include”, “containing”, “contains” and “contain” are inclusive or open-ended and do not exclude additional, unrecited elements or method steps.

Reference to “some embodiments”, “an embodiment”, “one embodiment”, or “other embodiments” means that a particular feature or characteristic described in connection with the embodiments is included in at least one or more embodiments, but not necessarily all embodiments, of the present disclosure.

The term “about” or “approximately” in relation to a numerical means, a range of values that fall within 10% greater than or less than the value. For example, about x means x±(10%*x).

The term “between” means the range of numbers including the first and the last number in a range.

As used herein, a “cell” generally refers to a biological cell. A cell can be the basic structural, functional and/or biological unit of a living organism. A cell can originate from any organism having one or more cells. Sometimes a cell may not originate from a natural organism (e.g., a cell can be synthetically made, sometimes termed an artificial cell).

In some embodiments, the cell is a human cell. A cell may be of or derived from different tissues, organs, and/or cell types. In some embodiments, the cell is a primary cell. As used herein, the term “primary cell” means a cell isolated from an organism, e.g., a mammal, which is grown in tissue culture (i.e., in vitro) for the first time before subdivision and transfer to a subculture. In some non-limiting examples, mammalian cells, including primary cells and stem cells can be modified through introduction of one or more polynucleotides, polypeptides, and/or prime editing compositions (e.g., through transfection, transduction, electroporation and the like) and further passaged.

Such modified cells include a T-cell, e.g., a primary T cell, e.g., an inflammatory T cell, a T helper cell, a cytotoxic T-cell, a CD4+ T-cell, a CD8+ T cell, a memory T cell, a regulatory T cell, a natural killer T cell, a mucosal associated invariant T cell, a γδ T cell, an alpha beta T cell, a naive T cell, or an effector T cell), formed elements of the blood (e.g., lymphocytes, bone marrow cells), precursors or progenitors thereof, differentiated or de-differentiated cell thereof, and stem cells. In some embodiments, a cell is a naive T cell (e.g., a naive CD8+ T cell). In some embodiments, the cell is a transformed T cell. In some embodiments, the cell is an immune cell (e.g., a primary immune cell) or a progenitor or a precursor thereof). In some embodiments, the cell is a T-cell, or a progenitor or a precursor thereof. In some embodiments, the cell is a human T cell, or a progenitor or a precursor thereof. In some embodiments, the cell is a T helper cell (e.g., Th1 cell, Th2 cell, Th9 cell, Th17 cell, Th22 cell, and Tfh (follicular helper) cell). In some embodiments, the cell is a cytotoxic T cell. In some embodiments, the cell is a CD8+ T cell. In some embodiments, the cell is a CD4+ T cell. In some embodiments, the cell is a memory T cell (e.g., (e.g., central memory T cell (T_{CM}), stem memory T cell (TSCM), effector memory T cell, Tissue resident memory T cell). In some embodiments, the cell is an effector memory T cell (e.g., TEM cells and TEMRA (CD45RA⁺) cells). In some embodiments, the cell is a regulatory T cell. In some embodiments, the cell is a natural killer T cell. In some embodiments, the cell is a Mucosal associated invariant T cell. In some embodiments, the cell is a γδ T cell. In some embodiments, the cell is an effector T cell. In some embodiments, the cell is a thymocyte. In some embodiments, the cell is a lymphoid cell. In some embodiments, the cell is a common lymphoid progenitor cells. In some embodiments, the cell is an early thymic progenitor cell. In some embodiments, the cell is a CD3+ cell. In some embodiments, the cell is a tumor infiltrating lymphocyte. In some embodiments, the cell is a myeloid cell. In some embodiments, the cell is a plasma cell. In some embodiments, the cell is an activated T cell.

In some embodiments, the cell is a stem cell (e.g., adult stem cell, embryonic stem cell, non-embryonic stem cell), cord blood stem cell, progenitor cell, bone marrow stem cell, induced pluripotent stem cell, totipotent stem cell, a CD34+ cell, or hematopoietic stem cell). In some embodiments, the cell is a pluripotent cell (e.g., a pluripotent stem cell). In some embodiments, the cell (e.g., a stem cell) is an embryonic stem cell, tissue-specific stem cell, mesenchymal stem cell, or an induced pluripotent stem cell. In some embodiments, the cell is an induced pluripotent stem cell (iPSC). In some embodiments, the cell is a hematopoietic stem cell. In some embodiments, the cell is a hematopoietic stem and progenitor cell. In some embodiments, the cell is a multipotent progenitor cell. In some embodiments, the cell is a T-cell progenitor. In some embodiments, the cell is a T-cell precursor. In some embodiments, the cell is an embryonic stem cell (ESC). In some embodiments, the cell is a human stem cell. In some embodiments, the cell is a human pluripotent stem cell. In some embodiments, the cell is a non-embryonic stem cell. In some embodiments, the cell is an induced human pluripotent stem cell. In some embodiments, the cell is a human stem cell. In some embodiments, the cell is a human embryonic stem cell. In some embodiments, the cell is a human T-cell progenitor. In some embodiments, the cell is a human T-cell precursor.

In some embodiments, the cell is a mammalian cell.

In some embodiments, a cell is not isolated from an organism but forms part of a tissue or organ of an organism, e.g., a mammal.

In some embodiments, the cell is a differentiated cell. In some embodiments, the cell is differentiated from an induced pluripotent stem cell. In some embodiments, the cell is a T-cell e.g., a primary T cell, e.g., an inflammatory T cell, a T helper cell, a cytotoxic T-cell, a CD4+ T-cell, a CD8+ T cell, a memory T cell, a regulatory T cell, a natural killer T cell, a mucosal associated invariant T cell, a γδ T cell, an alpha beta T cell, a naive T cell, or an effector T cell differentiated from an iPSC, ESC, a T-cell precursor, or a T-cell progenitor.

In some embodiments, the cell is a differentiated human cell. In some embodiments, the cell is differentiated from an induced human pluripotent stem cell. In some embodiments, the cell edited by prime editing can be differentiated into, or give rise to recovery of a population of cells, e.g., a T-cell e.g., a primary T cell, e.g., an inflammatory T cell, a T helper cell, a cytotoxic T-cell, a CD4+ T-cell, a CD8+ T cell, a memory T cell, a regulatory T cell, a natural killer T cell, a mucosal associated invariant T cell, a γδ T cell, an alpha beta T cell, a naive T cell, or an effector T cell. In some embodiments, the cell is in a subject, e.g., a human subject. In some embodiments, the cell is obtained from a subject prior to editing. For example, in some embodiments, the cell is obtained from a patient having a cancer, a microbial infection, a graft vs host disease, or an autoimmune disorder. Prior to editing by the methods and compositions disclosed herein the cell can be obtained from a subject through a variety of non-limiting methods. T cells can be obtained from a number of non-limiting sources, for example, peripheral blood mononuclear cells, bone marrow, lymph node tissue, cord blood, thymus tissue, tissue from a site of infection, ascites, pleural effusion, spleen tissue, and tumors. Methods of collecting blood cells, isolating and enriching T cells, and expanding them ex vivo may be by methods known in the art. In some embodiments, the cell can be obtained from a cell bank, a blood bank, cell culture, or any number of T cell lines available, and known to those skilled in the art. Cells may also be obtained from a tissue biopsy, surgery, blood, plasma, serum, or other biological fluid. In some embodiments, the cell can be obtained prior to editing from one or more healthy donors, from a patient having a cancer, a microbial infection, a graft versus host infection, or an autoimmune disorder.

For example, a cell can be obtained (i.e., isolated or purified) prior to editing from a whole blood sample by lysing red blood cells or a fractionated blood sample, and removing peripheral mononuclear blood cells by centrifugation. The cell can be further isolated or purified using a selective purification method that isolates the cell based on cell-specific markers such as CD25, CD3, CD4, CD8, CD28, CD45RA, or CD45RO (e.g., by flow cytometry). In one embodiment, CD4+ is used as a marker to select T cells. In one embodiment, CD8+ is used as a marker to select T cells. In one embodiment, CD4+ and CD8+ are used as a marker to select regulatory T cells.

In some embodiments, the edited cell produced using the methods and compositions disclosed herein are cultured, proliferated, expanded, differentiated, and or de-differentiated in vitro.

In some embodiments, the cell comprises a prime editor or a prime editing composition. In some embodiments, the cell comprises a dual prime editing composition or system comprising a prime editor and at least two PERNAs that are different from each other. In some embodiments, the cell is from a human subject. In some embodiments, the cell is from a human subject, and comprises a prime editor or a prime editing composition for editing a TRAC gene. In some embodiments, the cell is from a human subject and the TRAC gene has been edited by prime editing. In some embodiments, the cell comprises a prime-edited TRAC gene, and is administered to a subject (e.g., a human subject). In some embodiments, the cell is in a human subject, and comprises a prime editor or a prime editing composition for editing a TRAC gene. In some embodiments, the cell is from the human subject and the TRAC gene has been edited or corrected by prime editing. In some embodiments, the human subject is a healthy donor, or has a disease, disorder, or a condition, e.g., a cancer, a microbial infection, an autoimmune disorder, a T cell malignancy, or a graft versus host disorder. In some embodiments, the human subject is in need of, or is undergoing, or will be undergoing an immune cell immunotherapy (e.g., a T cell therapy such as a CAR-T cell therapy).

The term “substantially” as used herein may refer to a value approaching 100% of a given value. In some embodiments, the term may refer to an amount that may be at least about 70%, 80%, 90%, 91%, 92%, 93%, 94%, 95%, 96%, 97%, 98%, 99%, 99.9%, or 99.99% of a total amount. In some embodiments, the term may refer to an amount that may be about 100% of a total amount.

The terms “protein” and “polypeptide” can be used interchangeably to refer to a polymer of two or more amino acids joined by covalent bonds (e.g., an amide bond) that can adopt a three-dimensional conformation. In some embodiments, a protein or polypeptide comprises at least 10 amino acids, 15 amino acids, 20 amino acids, 30 amino acids or 50 amino acids joined by covalent bonds (e.g., amide bonds). In some embodiments, a protein comprises at least two amide bonds. In some embodiments, a protein comprises multiple amide bonds. In some embodiments, a protein comprises an enzyme, enzyme precursor proteins, regulatory protein, structural protein, receptor, nucleic acid binding protein, a biomarker, a member of a specific binding pair (e.g., a ligand or aptamer), or an antibody. In some embodiments, a protein may be a full-length protein (e.g., a fully processed protein having certain biological function). In some embodiments, a protein may be a variant or a fragment of a full-length protein. For example, in some embodiments, a Cas9 protein domain comprises an H840A amino acid substitution compared to a naturally occurring S. pyogenes Cas9 protein. A variant of a protein or enzyme, for example a variant reverse transcriptase, comprises a polypeptide having an amino acid sequence that is about 60% identical, about 70% identical, about 80% identical, about 90% identical, about 95% identical, about 96% identical, about 97% identical, about 98% identical, about 99% identical, about 99.5% identical, or about 99.9% identical to the amino acid sequence of a reference protein.

In some embodiments, a protein comprises one or more protein domains or subdomains. As used herein, the term “polypeptide domain”, “protein domain”, or “domain” when used in the context of a protein or polypeptide, refers to a polypeptide chain that has one or more biological functions, e.g., a catalytic function, a protein-protein binding function, or a protein-DNA function. In some embodiments, a protein comprises multiple protein domains. In some embodiments, a protein comprises multiple protein domains that are naturally occurring. In some embodiments, a protein comprises multiple protein domains from different naturally occurring proteins. For example, in some embodiments, a prime editor may be a fusion protein comprising a Cas9 protein domain of S. pyogenes and a reverse transcriptase protein domain of a retrovirus (e.g., a Moloney murine leukemia virus) or a variant of the retrovirus. A protein that comprises amino acid sequences from different origins or naturally occurring proteins may be referred to as a fusion, or chimeric protein.

In some embodiments, a protein comprises a functional variant or functional fragment of a full-length wild-type protein. A “functional fragment” or “functional portion”, as used herein, refers to any portion of a reference protein (e.g., a wild-type protein) that encompasses less than the entire amino acid sequence of the reference protein while retaining one or more of the functions, e.g., catalytic or binding functions. For example, a functional fragment of a reverse transcriptase may encompass less than the entire amino acid sequence of a wild-type reverse transcriptase, but retains the ability under at least one set of conditions to catalyze the polymerization of a polynucleotide. When the reference protein is a fusion of multiple functional domains, a functional fragment thereof may retain one or more of the functions of at least one of the functional domains. For example, a functional fragment of a Cas9 may encompass less than the entire amino acid sequence of a wild-type Cas9, but retains its DNA binding ability and lacks its nuclease activity partially or completely.

A “functional variant” or “functional mutant”, as used herein, refers to any variant or mutant of a reference protein (e.g., a wild-type protein) that encompasses one or more alterations to the amino acid sequence of the reference protein while retaining one or more of the functions, e.g., catalytic or binding functions. In some embodiments, the one or more alterations to the amino acid sequence comprises amino acid substitutions, insertions or deletions, or any combination thereof. In some embodiments, the one or more alterations to the amino acid sequence comprises amino acid substitutions. For example, a functional variant of a reverse transcriptase may comprise one or more amino acid substitutions compared to the amino acid sequence of a wild-type reverse transcriptase, but retains the ability under at least one set of conditions to catalyze the polymerization of a polynucleotide. When the reference protein is a fusion of multiple functional domains, a functional variant thereof may retain one or more of the functions of at least one of the functional domains. For example, in some embodiments, a functional variant of a Cas9 may comprise one or more amino acid substitutions in a nuclease domain, e.g., an H840A amino acid substitution, compared to the amino acid sequence of a wild-type Cas9, but retains the DNA binding ability and lacks the nuclease activity partially or completely.

The term “function” and its grammatical equivalents as used herein may refer to a capability of operating, having, or serving an intended purpose. Functional may comprise any percent from baseline to 100% of an intended purpose. For example, functional may comprise or comprise about 5%, 10%, 15%, 20%, 25%, 30%, 35%, 40%, 45%, 50%, 55%, 60%, 65%, 70%, 75%, 80%, 85%, 90%, 95%, or up to about 100% of an intended purpose. In some embodiments, the term functional may mean over or over about 100% of normal function, for example, 125%, 150%, 175%, 200%, 250%, 300%, 400%, 500%, 600%, 700% or up to about 1000% of an intended purpose.

In some embodiments, a protein or polypeptide includes naturally occurring amino acids (e.g., one of the twenty amino acids commonly found in peptides synthesized in nature, and known by the one letter abbreviations A, R, N, C, D, Q, E, G, H, I, L, K, M, F, P, S, T, W, Y and V). In some embodiments, a protein or polypeptides includes non-naturally occurring amino acids (e.g., amino acids which is not one of the twenty amino acids commonly found in peptides synthesized in nature, including synthetic amino acids, amino acid analogs, and amino acid mimetics). In some embodiments, a protein or polypeptide is modified.

In some embodiments, a protein comprises an isolated polypeptide. The term “isolated” means free or removed to varying degrees from components which normally accompany it as found in the natural state or environment. For example, a polypeptide naturally present in a living animal is not isolated, and the same polypeptide partially or completely separated from the coexisting materials of its natural state is isolated.

In some embodiments, a protein is present within a cell, a tissue, an organ, or a virus particle. In some embodiments, a protein is present within a cell or a part of a cell (e.g., a bacteria cell, a plant cell, or an animal cell). In some embodiments, the cell is in a tissue, in a subject, or in a cell culture. In some embodiments, the cell is a microorganism (e.g., a bacterium, fungus, protozoan, or virus). In some embodiments, a protein is present in a mixture of analytes (e.g., a lysate). In some embodiments, the protein is present in a lysate from a plurality of cells or from a lysate of a single cell.

The terms “homologous,” “homology,” or “percent homology” as used herein refer to the degree of sequence identity between an amino acid and a corresponding reference amino acid sequence or a polynucleotide sequence and a corresponding reference polynucleotide sequence. “Homology” can refer to polymeric sequences, e.g., polypeptide or DNA sequences that are similar. Homology can mean, for example, nucleic acid sequences with at least about: 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity. In other embodiments, a “homologous sequence” of nucleic acid sequences may exhibit 93%, 95% or 98% sequence identity to the reference nucleic acid sequence. For example, a “region of homology to a genomic region” can be a region of DNA that has a similar sequence to a given genomic region in the genome. A region of homology can be of any length that is sufficient to promote binding of a spacer, or a primer binding site to the complementary sequence of a genomic region. For example, the region of homology can comprise at least 5, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100 or more bases in length such that the region of homology has sufficient homology to undergo binding with the corresponding genomic region.

When a percentage of sequence homology or identity is specified, in the context of two nucleic acid sequences or two polypeptide sequences, the percentage of homology or identity generally refers to the alignment of two or more sequences across a portion of their length when compared and aligned for maximum correspondence. When a position in the compared sequence can be occupied by the same base or amino acid, then the molecules can be homologous at that position. Unless stated otherwise, sequence homology or identity is assessed over the specified length of the nucleic acid, polypeptide or portion thereof. In some embodiments, the homology or identity is assessed over a functional portion or specified portion of the length.

Alignment of sequences for assessment of sequence homology can be conducted by algorithms known in the art, such as the Basic Local Alignment Search Tool (BLAST) algorithm, which is described in Altschul et al, J. Mol. Biol. 215:403-410, 1990. A publicly available, internet interface, for performing BLAST analyses is accessible through the National Center for Biotechnology Information. Additional known algorithms include those published in: Smith & Waterman, “Comparison of Biosequences”, Adv. Appl. Math. 2:482, 1981; Needleman & Wunsch, “A general method applicable to the search for similarities in the amino acid sequence of two proteins” J. Mol. Biol. 48:443, 1970; Pearson & Lipman “Improved tools for biological sequence comparison”, Proc. Natl. Acad. Sci. USA 85:2444, 1988; or by automated implementation of these or similar algorithms. Global alignment programs may also be used to align similar sequences of roughly equal size. Examples of global alignment programs include NEEDLE (available at www.ebi.ac.uk/Tools/psa/emboss_needle/) which is part of the EMBOSS package (Rice P et al., Trends Genet., 2000; 16:276-277), and the GGSEARCH program https://fasta.bioch.virginia.edu/fasta_www2/, which is part of the FASTA package (Pearson W and Lipman D, 1988, Proc. Natl. Acad. Sci. USA, 85:2444-2448). Both of these programs are based on the Needleman-Wunsch algorithm which is used to find the optimum alignment (including gaps) of two sequences along their entire length. A detailed discussion of sequence analysis can also be found in Unit 19.3 of Ausubel et al (“Current Protocols in Molecular Biology” John Wiley & Sons Inc, 1994-1998, Chapter 15, 1998). In some embodiments, alignment between a query sequence and a reference sequence is performed with Needleman-Wunsch alignment with Gap Costs set to Existence: 11 Extension: 1 where percent identity is calculated by dividing the number of identities by the length of the alignment, as further described in Altschul et al. (“Gapped BLAST and PSI-BLAST: a new generation of protein database search programs”, Nucleic Acids Res. 25:3389-3402, 1997) and Altschul et al, (“Protein database searches using compositionally adjusted substitution matrices”, FEBS J. 272:5101-5109, 2005).

A skilled person understands that amino acid (or nucleotide) positions may be determined in homologous sequences based on alignment, for example, “H840” in a reference Cas9 sequence may correspond to H839, or another position in a Cas9 homolog.

The term “polynucleotide” or “nucleic acid molecule” can be any polymeric form of nucleotides, including DNA, RNA, a hybridization thereof, or RNA-DNA chimeric molecules. In some embodiments, a polynucleotide comprises cDNA, genomic DNA, mRNA, tRNA, rRNA, or microRNA. In some embodiments, a polynucleotide is double-stranded, e.g., a double-stranded DNA in a gene. In some embodiments, a polynucleotide is single-stranded or substantially single-stranded, e.g., single-stranded DNA or an mRNA. In some embodiments, a polynucleotide is a cell-free nucleic acid molecule. In some embodiments, a polynucleotide circulates in blood. In some embodiments, a polynucleotide is a cellular nucleic acid molecule. In some embodiments, a polynucleotide is a cellular nucleic acid molecule in a cell circulating in blood.

Polynucleotides can have any three-dimensional structure. The following are nonlimiting examples of polynucleotides: a gene or gene fragment (for example, a probe, primer, EST or SAGE tag), an exon, an intron, intergenic DNA (including, without limitation, heterochromatic DNA), messenger RNA (mRNA), transfer RNA (tRNA), ribosomal RNA (rRNA), a ribozyme, cDNA, a recombinant polynucleotide, a branched polynucleotide, a plasmid, a vector, isolated DNA, isolated RNA, sgRNA, guide RNA, a nucleic acid probe, a primer, an snRNA, a long non-coding RNA, a snoRNA, a siRNA, a miRNA, a tRNA-derived small RNA (tsRNA), an antisense RNA, an shRNA, or a small rDNA-derived RNA (srRNA).

In some embodiments, a polynucleotide comprises deoxyribonucleotides, ribonucleotides or analogs thereof. In some embodiments, a polynucleotide comprises modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component.

In some embodiments, a polynucleotide is composed of a specific sequence of four nucleotide bases: adenine (A); cytosine (C); guanine (G); thymine (T); and uracil (U) for thymine when the polynucleotide is RNA. In some embodiments, the polynucleotide may comprise one or more other nucleotide bases, such as inosine (I), which is read by the translation machinery as guanine (G).

In some embodiments, a polynucleotide may be modified. As used herein, the terms “modified” or “modification” refers to chemical modification with respect to the A, C, G, T and U nucleotides. In some embodiments, modifications may be on the nucleoside base and/or sugar portion of the nucleosides that comprise the polynucleotide. In some embodiments, the modification may be on the internucleoside linkage (e.g., phosphate backbone). In some embodiments, multiple modifications are included in the modified nucleic acid molecule. In some embodiments, a single modification is included in the modified nucleic acid molecule.

The term “complement”, “complementary”, or “complementarity” as used herein, refers to the ability of two polynucleotide molecules to base pair with each other. Complementary polynucleotides may base pair via hydrogen bonding, which may be Watson Crick, Hoogsteen or reversed Hoogsteen hydrogen bonding. For example, an adenine on one polynucleotide molecule will base pair to a thymine or an uracil on a second polynucleotide molecule and a cytosine on one polynucleotide molecule will base pair to guanine on a second polynucleotide molecule. Two polynucleotide molecules are complementary to each other when a first polynucleotide molecule comprising a first nucleotide sequence can base pair with a second polynucleotide molecule comprising a second nucleotide sequence. For instance, the two DNA molecules 5′-ATGC-3′ and 5′-GCAT-3′ are complementary, and the complement of the DNA molecule 5′-ATGC-3′ is 5′-GCAT-3′. A percentage of complementarity indicates the percentage of nucleotides in a polynucleotide molecule which can base pair with a second polynucleotide molecule (e.g., 5, 6, 7, 8, 9, 10 out of 10 being 50%, 60%, 70%, 80%, 90%, and 100% complementary, respectively). “Perfectly complementary” means that all the contiguous nucleotides of a polynucleotide molecule will base pair with the same number of contiguous nucleotides in a second polynucleotide molecule. “Substantially complementary” as used herein refers to a degree of complementarity that can be 70%, 75%, 80%, 85%, 90%, 95%, 97%, 98%, or 99% over all or a portion of two polynucleotide molecules. In some embodiments, the portion of complementarity may be a region of 10, 15, 20, 25, 30, 35, 40, 45, 50, or more nucleotides. “Substantial complementary” can also refer to a 100% complementarity over a portion or a region of two polynucleotide molecules. In some embodiments, the portion or the region of complementarity between the two polynucleotide molecules is at least 60%, 65%, 70%, 75%, 80%, 85%, 90%, 95%, 97%, 98%, or 99% of the length of at least one of the two polynucleotide molecules or a functional or defined portion thereof.

As used herein, “expression” refers to the process by which polynucleotides are transcribed into mRNA and/or the process by which polynucleotides, e.g., the transcribed mRNA, are translated into peptides, polypeptides, or proteins. If the polynucleotide is derived from genomic DNA, expression may include splicing of the mRNA in a eukaryotic cell. In some embodiments, expression of a polynucleotide, e.g., a gene or a DNA encoding a protein, is determined by the amount of the protein encoded by the gene after transcription and translation of the gene. In some embodiments, expression of a polynucleotide, e.g., a gene or a DNA encoding a protein, is determined by the amount of a functional form of the protein encoded by the gene after transcription and translation of the gene. In some embodiments, expression of a gene is determined by the amount of the mRNA, or transcript that is encoded by the gene after transcription of the gene. In some embodiments, expression of a polynucleotide, e.g., an mRNA, is determined by the amount of the protein encoded by the mRNA after translation of the mRNA. In some embodiments, expression of a polynucleotide, e.g., an mRNA or coding RNA, is determined by the amount of a functional form of the protein encoded by the polypeptide after translation of the polynucleotide.

The term “sequencing” as used herein, may comprise capillary sequencing, bisulfite-free sequencing, bisulfite sequencing, TET-assisted bisulfite (TAB) sequencing, ACE-sequencing, high-throughput sequencing, Maxam-Gilbert sequencing, massively parallel signature sequencing, Polony sequencing, 454 pyrosequencing, Sanger sequencing, Illumina sequencing, SOLID sequencing, Ion Torrent semiconductor sequencing, DNA nanoball sequencing, Heliscope single molecule sequencing, single molecule real time (SMRT) sequencing, nanopore sequencing, shot gun sequencing, RNA sequencing, or any combination thereof.

The terms “equivalent” or “biological equivalent” are used interchangeably when referring to a particular molecule, or biological or cellular material, and means a molecule having minimal homology to another molecule while still maintaining a desired structure or functionality.

The term “encode” as it is applied to polynucleotides refers to a polynucleotide which is said to “encode” another polynucleotide, a polypeptide, or an amino acid if, in its native state or when manipulated by methods well known to those skilled in the art, it can be used as a polynucleotide synthesis template, e.g., transcribed into an RNA, reverse transcribed into a DNA or cDNA, and/or translated to produce an amino acid, or a polypeptide or fragment thereof. In some embodiments, a polynucleotide comprising three contiguous nucleotides form a codon that encodes a specific amino acid. In some embodiments, a polynucleotide comprises one or more codons that encode a polypeptide. In some embodiments, a polynucleotide comprising one or more codons comprises a mutation in a codon compared to a wild-type reference polynucleotide. In some embodiments, the mutation in the codon encodes an amino acid substitution in a polypeptide encoded by the polynucleotide as compared to a wild-type reference polypeptide.

The term “mutation” as used herein refers to a change and/or alteration in an amino acid sequence of a protein or nucleic acid sequence of a polynucleotide. Such changes and/or alterations may comprise the substitution, insertion, deletion and/or truncation of one or more amino acids, in the case of an amino acid sequence, and/or nucleotides, in the case of nucleic acid sequence, compared to a reference amino acid or a reference nucleic acid sequence. In some embodiments, the reference sequence is a wild-type sequence. In some embodiments, a mutation in a nucleic acid sequence of a polynucleotide encodes a mutation in the amino acid sequence of a polypeptide. In some embodiments, the mutation in the amino acid sequence of the polypeptide or the mutation in the nucleic acid sequence of the polynucleotide is a mutation associated with a disease state.

The term “subject” and its grammatical equivalents as used herein may refer to a human or a non-human. A subject may be a mammal. A human subject may be male or female. A human subject may be of any age. A subject may be a human embryo. A human subject may be a newborn, an infant, a child, an adolescent, or an adult. A human subject may be in need of treatment for a genetic disease or disorder. A human subject may be in need of a cell therapy., e.g., an immune cell immunotherapy such as a T cell therapy). A human subject may be in need of a CAR-T cell therapy.

The terms “treatment” or “treating” and their grammatical equivalents may refer to the medical management of a subject with an intent to cure, ameliorate, or ameliorate a symptom of, a disease, condition, or disorder. Treatment may include active treatment, that is, treatment directed specifically toward the improvement of a disease, condition, or disorder. Treatment may include causal treatment, that is, treatment directed toward removal of the cause of the associated disease, condition, or disorder. In addition, this treatment may include palliative treatment, that is, treatment designed for the relief of symptoms rather than the curing of the disease, condition, or disorder. Treatment may include supportive treatment, that is, treatment employed to supplement another specific therapy directed toward the improvement of the disease, condition, or disorder. In some embodiments, a condition may be pathological. In some embodiments, a treatment may not completely cure or prevent a disease, condition, or disorder. In some embodiments, a treatment ameliorates, but does not completely cure or prevent a disease, condition, or disorder. In some embodiments, a subject may be treated for 12 hours, 24 hours, 2 days, 3 days, 4 days, 5 days, 6 days, 7 days, 2 weeks, 3 weeks, 4 weeks, 2 months, 3 months, 4 months, 5 months, 6 months, 1 year, 2 years, 3 years, 4 years, 5 years, 6 years, indefinitely, or life of the subject.

The term “ameliorate” and its grammatical equivalents means to decrease, suppress, attenuate, diminish, arrest, or stabilize the development or progression of a disease.

The terms “prevent” or “preventing” means delaying, forestalling, or avoiding the onset or development of a disease, condition, or disorder for a period of time. Prevent also means reducing risk of developing a disease, disorder, or condition. Prevention includes minimizing or partially or completely inhibiting the development of a disease, condition, or disorder. In some embodiments, a composition, e.g., a pharmaceutical composition, prevents a disorder by delaying the onset of the disorder for 12 hours, 24 hours, 2 days, 3 days, 4 days, 5 days, 6 days, 7 days, 2 weeks, 3 weeks, 4 weeks, 2 months, 3 months, 4 months, 5 months, 6 months, 1 year, 2 years, 3 years, 4 years, 5 years, 6 years, indefinitely, or life of a subject.

The term “effective amount” or “therapeutically effective amount” refers to a quantity of a composition, for example a prime editing composition comprising a construct, that can be sufficient to result in a desired activity upon introduction into a subject as disclosed herein. An effective amount of the prime editing compositions can be provided to the target gene or cell, whether the cell is in vitro, ex vivo or in vivo.

An effective amount can be the amount to induce, for example, at least about a 2-fold change (increase or decrease) or more in the amount of target nucleic acid modulation observed relative to a negative control. An effective amount or dose can induce, for example, about 2-fold increase, about 3-fold increase, about 4-fold increase, about 5-fold increase, about 6-fold increase, about 7-fold increase, about 8-fold increase, about 9-fold increase, about 10-fold increase, about 25-fold increase, about 50-fold increase, about 100-fold increase, about 200-fold increase, about 500-fold increase, about 700-fold increase, about 1000-fold increase, about 5000-fold increase, or about 10,000-fold increase in target gene modulation.

The amount of target gene modulation may be measured by any suitable method known in the art. In some embodiments, the “effective amount” or “therapeutically effective amount” is the amount of a composition that is required to ameliorate the symptoms of a disease relative to an untreated patient. In some embodiments, an effective amount is the amount of a composition sufficient to introduce an alteration in a gene of interest (e.g., a TRAC gene) in a cell (e.g., a cell in vitro, ex vivo or in vivo).

As it relates to the edited cells produced by the methods and composition comprising the edited cells disclosed herein; a “therapeutically effective amount” refers to a quantity of a composition comprising the edited cells that can be sufficient to result in a desired activity upon introduction into a subject (e.g., a human subject).

The term “construct” refers to a polynucleotide or a portion of a polynucleotide, comprising one or more nucleic acid sequences encoding one or more transcriptional products and/or proteins. A construct may be a recombinant nucleic acid molecule or a part thereof. In some embodiments, the one or more nucleic acid sequences of a construct are operably linked to one or more regulatory sequences, for example, transcriptional initiation regulatory sequences. In some embodiments, a construct is a vector, a plasmid, or a portion thereof. In some embodiments, a construct a construct comprises DNA. In some embodiments, a construct comprises RNA. In some embodiments, a construct is double-stranded. In some embodiments, a construct is single-stranded. In some embodiments, a construct comprises an expression cassette. An expression cassette means a polynucleotide comprising a nucleic acid sequence that encodes one or more transcriptional products and is operably linked to at least one transcriptional regulatory sequence, e.g., a promoter.

The term “exogenous” when used in reference to a biomolecule, e.g., a polynucleotide sequence or a polypeptide sequence refers to a biomolecule that is not native to a specific biological context, e.g., a gene, a particular chromosome, a particular cell or chromosomal site of the cell, tissue, or organism, or, if from the same source, is modified from its original form or is present in a non-native location, e.g., a chromosome location.

The term “endogenous” when used in reference to a biomolecule, e.g., a polynucleotide sequence or a polypeptide sequence refers to a biomolecule that is native to or naturally occurring in a specific biological context, e.g., a gene, a particular chromosome, a particular cell or chromosomal site of the cell, tissue, or organism. For example, an endogenous sequence may be a wild-type sequence or may comprise one or more mutations compared to a wild-type sequence. In some embodiments, an endogenous sequence is mutated compared to a wild-type sequence and may cause or be associated with a disease or disorder in a subject. As used herein, in some embodiments, a wild-type sequence, with respect to a specific gene and a specific disease, is a gene sequence found in healthy individuals, wherein the wild-type sequence does not include a mutation causative of the specific disease.

The term “recombinase,” as used herein, refers to a site-specific enzyme that mediates the recombination of DNA between recombinase recognition sequences, which results in the excision, integration, inversion, or exchange (e.g., translocation) of DNA fragments between the recombinase recognition sequences. Recombinases can be classified into two distinct families: serine recombinases (e.g., resolvases and invertases) and tyrosine recombinases (e.g., integrases). Examples of serine recombinases include, without limitation, Si74, No67, Kp03, Pa01, Nm60, BceINTa, BcytINTd, SscINTd, SacINTd, Hin, Gin, Tn3, 13-six, CinH, ParA, y6, Bxbl, OC31, TP901, TG1, pBT1, R4, pRV1, pFC1, MR11, A118, U153, and gp29. Examples of tyrosine recombinases include, without limitation, Cre, FLP, R, Lambda, HK101, HK022, and pSAM2. Recombinases have numerous applications, including the creation of gene knockouts/knock-ins and gene therapy applications, as described in international publication no. WO2020191248A1, which is hereby incorporated by reference in its entirety. The recombinases provided herein are not meant to be exclusive examples of recombinases that can be used in embodiments of the invention. The methods and compositions of the invention can be expanded by mining databases for new orthogonal recombinases or designing synthetic recombinases with defined DNA specificities.

In some embodiments, the catalytic domains of a recombinase are fused to a programmable DNA binding domain of a prime editor, such as a RNA-programmable nuclease (e.g., dCas9, Cas9 nickase, or a fragment thereof), such that the recombinase domain does not comprise a nucleic acid binding domain or is unable to bind to a target nucleic acid (e.g., the recombinase domain is engineered such that it does not have specific DNA binding activity). For example, serine recombinases of the resolvase-invertase group, e.g., Tn3 and 76 resolvases and the Hin and Gin invertases, have modular structures with autonomous catalytic and DNA-binding domains. The catalytic domains of these recombinases are thus amenable to being combined with, e.g., fused to or connected to a prime editor or a component thereof, as described herein, e.g., following the isolation of ‘activated’ recombinase mutants which do not require any accessory factors (e.g., DNA binding activities).

Additionally, many other natural serine recombinases having an N-terminal catalytic domain and a C-terminal DNA binding domain are known (e.g., phiC31 integrase, TnpX transposase, IS607 transposase), and their catalytic domains can be co-opted to engineer programmable site-specific recombinases as described herein. Similarly, the core catalytic domains of tyrosine recombinases (e.g., Cre, integrase) are known, and can be similarly co-opted to engineer programmable site-specific recombinases as described herein.

Other examples of recombinases that are useful in the methods and compositions described herein are known to those of skill in the art, and any new recombinase that is discovered or generated is expected to be able to be used in the different embodiments of the invention.

The term “recombinase recognition sequence”, or equivalently as “RRS” or “recombinase target sequence” or “recombinase site,” as used herein, refers to a nucleotide sequence target recognized by a recombinase and which undergoes strand exchange with another DNA molecule having a the RRS that results in excision, integration, inversion, or exchange of DNA fragments between the recombinase recognition sequences. In various embodiments, a prime editing composition may install one or more recombinase sites in a target sequence, or in more than one target sequence. When more than one recombinase site is installed by prime editing, the recombinase sites can be installed at adjacent target sites or non-adjacent target sites (e.g., separate chromosomes). In various embodiments, single installed recombinase sites can be used as “landing sites” for a recombinase-mediated reaction between the genomic recombinase site and a second recombinase site within an exogenously supplied nucleic acid molecule, e.g., a plasmid or a DNA vector. This enables the targeted integration of a desired nucleic acid molecule.

The term “recombine,” or “recombination,” in the context of a nucleic acid modification (e.g., a genomic modification), is used to refer to the process by which two or more nucleic acid molecules, or two or more regions of a single nucleic acid molecule, are modified by the action of a recombinase protein (e.g., an inventive recombinase fusion protein provided herein). Recombination can result in, inter alia, the insertion, inversion, excision, or translocation of nucleic acids, e.g., in or between one or more nucleic acid molecules.

### Prime Editing and Dual Prime Editing

The term “prime editing” refers to programmable editing of a target DNA using a prime editor complexed with a PERNA to incorporate an intended nucleotide edit (also referred to herein as a nucleotide change) into the target DNA through target-primed DNA synthesis. In prime editing, a target DNA may comprise a double-stranded DNA molecule having two complementary strands. When viewed in the context of each specific PEgRNA, the two complementary strands of a double-stranded target DNA may comprise a first strand that may be referred to as a “target strand” or a “non-edit strand”, and a second strand that may be referred to as a “non-target strand,” or an “edit strand.” In some embodiments, in a prime editing guide RNA (PEgRNA), a spacer sequence is complementary or substantially complementary to a specific sequence on the target strand, which may be referred to as a “search target sequence”. In some embodiments, the spacer sequence anneals with the target strand at the search target sequence. The target strand may also be referred to as the “non-Protospacer Adjacent Motif (non-PAM strand).” In some embodiments, the non-target strand may also be referred to as the “PAM strand”. In some embodiments, the PAM strand comprises a protospacer sequence and optionally a protospacer adjacent motif (PAM) sequence. In prime editing using a Cas-protein-based prime editor, a PAM sequence refers to a short DNA sequence immediately adjacent to the protospacer sequence on the PAM strand of the target gene. A PAM sequence may be specifically recognized by a programmable DNA binding protein, e.g., a Cas nickase or a Cas nuclease. In some embodiments, a specific PAM is characteristic of a specific programmable DNA binding protein, e.g., a Cas nickase or a Cas nuclease. A protospacer sequence refers to a specific sequence in the PAM strand of the target gene that is complementary to the search target sequence. In a PEgRNA, a spacer sequence may have a substantially identical sequence as the protospacer sequence on the edit strand of a target gene, except that the spacer sequence may comprise uracil (U) and the protospacer sequence may comprise thymine (T).

In some embodiments, the double stranded target DNA comprises a nick site on the PAM strand (or non-target strand). As used herein, a “nick site” refers to a specific position in between two nucleotides or two base pairs of the double stranded target DNA. In some embodiments, the position of a nick site is a specific position relative to the position of a specific PAM sequence. In some embodiments, the nick site is the particular position where a nick will occur when the double stranded target DNA is contacted with a nickase, for example, a Cas nickase, that recognizes a specific PAM sequence. In some embodiments, the nick site is upstream of a specific PAM sequence on the PAM strand of the double stranded target DNA. In some embodiments, the nick site is downstream of a specific PAM sequence on the PAM strand of the double stranded target DNA. In some embodiments, the nick site is upstream of a PAM sequence recognized by a Cas9 nickase, wherein the Cas9 nickase comprises a nuclease active RuvC domain and a nuclease inactive HNH domain. In some embodiments, the nick site is 3 nucleotides upstream of the PAM sequence, and the PAM sequence is recognized by a Streptococcus pyogenes Cas9 nickase, a P. lavamentivorans Cas9 nickase, a C. diphtheriae Cas9 nickase, a N. cinerea Cas9, a S. aureus Cas9, or a N. lari Cas9 nickase. In some embodiments, the nick site is 3 nucleotides upstream of the PAM sequence, and the PAM sequence is recognized by a Cas9 nickase, wherein the Cas9 nickase that comprises a nuclease active RuvC domain and a nuclease inactive HNH domain. In some embodiments, the nick site is 2 nucleotides upstream of the PAM sequence, and the PAM sequence is recognized by a S. thermophilus Cas9 nickase that comprises a nuclease active RuvC domain and a nuclease inactive HNH domain.

A “primer binding site” (also referred to as PBS or primer binding site sequence) is a single-stranded portion of the PEgRNA that comprises a region of complementarity to the PAM strand (i.e. the non-target strand or the edit strand). The PBS is complementary or substantially complementary to a sequence on the PAM strand of the double stranded target DNA that is immediately upstream of the nick site. In some embodiments, in the process of prime editing, the PERNA complexes with and directs a prime editor to bind the search target sequence on the target strand of the double stranded target DNA, and generates a nick at the nick site on the non-target strand of the double stranded target DNA. In some embodiments, the PBS is complementary to or substantially complementary to, and can anneal to, a free 3′ end on the non-target strand of the double stranded target DNA at the nick site. In some embodiments, the PBS annealed to the free 3′ end on the non-target strand can initiate target-primed DNA synthesis.

An “editing template” of a PEgRNA is a single-stranded portion of the PERNA that is 5′ of the PBS and which encodes a single strand of DNA. The editing template may comprise a region of complementarity to the PAM strand (i.e., the non-target strand or the edit strand), and comprises one or more intended nucleotide edits compared to the endogenous sequence of the double stranded target DNA. In some embodiments, the editing template and the PBS are immediately adjacent to each other. Accordingly, in some embodiments, a PEgRNA in prime editing comprises a single-stranded portion that comprises the PBS and the editing template immediately adjacent to each other. In some embodiments, the single stranded portion of the PEgRNA comprising both the PBS and the editing template is complementary or substantially complementary to an endogenous sequence on the PAM strand (i.e., the non-target strand or the edit strand) of the double stranded target DNA except for one or more non-complementary nucleotides at the intended nucleotide edit position(s). As used herein, regardless of relative 5′-3′ positioning in other context, the relative positions as between the PBS and the editing template, and the relative positions as among elements of a PEgRNA, are determined by the 5′ to 3′ order of the PERNA as a single molecule regardless of the position of sequences in the double stranded target DNA that may have complementarity or identity to elements of the PERNA. In some embodiments, the editing template is complementary or substantially complementary to a sequence on the PAM strand that is immediately downstream of the nick site, except for one or more non-complementary nucleotides at the intended nucleotide edit positions. The endogenous, e.g., genomic, sequence that is complementary or substantially complementary to the editing template, except for the one or more non-complementary nucleotides at the position corresponding to the intended nucleotide edit, may be referred to as an “editing target sequence”. In some embodiments, the editing template has identity or substantial identity to a sequence on the target strand that is complementary to, or having the same position in the genome as, the editing target sequence, except for one or more one or more insertions, deletions, or substitutions at the intended nucleotide edit positions. In some embodiments, the editing template encodes a single stranded DNA, wherein the single stranded DNA has identity or substantial identity to the editing target sequence except for one or more insertions, deletions, or substitutions at the positions of the one or more intended nucleotide edits. In some embodiments, the editing template may encode the wild-type or non-disease associated gene sequence (or its complement if the edit strand is the antisense strand of a gene). In some embodiments, the editing template may encode the wild-type or non-disease associated protein, but contain one or more synonymous mutations relative to the wild-type or non-disease associated protein coding region. Such synonymous mutations may include, for example, mutations that decrease the ability of a PEgRNA to rebind to the same target sequence once the desired edit is installed in the genome (e.g., synonymous mutations that silence the endogenous PAM sequence or that edit the endogenous protospacer).

In some embodiments, a PEgRNA complexes with and directs a prime editor to bind to the search target sequence of the target gene. In some embodiments, the bound prime editor generates a nick on the edit strand (PAM strand) of the target gene at the nick site. In some embodiments, a primer binding site (PBS) of the PEgRNA anneals with a free 3′ end formed at the nick site, and the prime editor initiates DNA synthesis from the nick site, using the free 3′ end as a primer. Subsequently, a single-stranded DNA encoded by the editing template of the PEgRNA is synthesized. In some embodiments, the newly synthesized single-stranded DNA comprises one or more intended nucleotide edits compared to the endogenous target gene sequence. Accordingly, in some embodiments, the editing template of a PEgRNA is complementary to a sequence in the edit strand except for one or more mismatches at the intended nucleotide edit positions in the editing template. The endogenous, e.g., genomic, sequence that is partially complementary to the editing template may be referred to as an “editing target sequence”. Accordingly, in some embodiments, the newly synthesized single stranded DNA has identity or substantial identity to a sequence in the editing target sequence, except for one or more insertions, deletions, or substitutions at the intended nucleotide edit positions. In some embodiments, the editing template comprises at least 4 contiguous nucleotides of complementarity with the edit strand wherein the at least 4 nucleotides contiguous are located upstream of the 5′ most edit in the editing template.

In some embodiments, prime editing may comprise programmable editing of a target DNA using one or more prime editors each complexed with a PEgRNA (“dual prime editing”). Dual prime editing refers to programmable editing of a double-stranded target DNA using two or more PEgRNAs, each of which is complexed with a prime editor for incorporating one or more intended nucleotide edits into the double-stranded target DNA. In some embodiments, dual prime editing incorporates one or more intended nucleotide edits into a double-stranded target DNA through excision of an endogenous DNA segment and/or replacement of the endogenous DNA segment with newly synthesized DNA via target-primed DNA synthesis. In some embodiments, dual prime editing may be used to edit a target DNA that is or is part of a target gene. In some embodiments, the target gene is a disease-associated gene. In some embodiments, the target gene is a monogenic disease-associated gene. In some embodiments, the target gene is a polygenic disease-associated gene. In some embodiments, the target gene is mutated compared to a wild-type sequence of the same gene and may cause or be associated with a disease or disorder in a subject. In some embodiments, the mutated target gene causes a disease or a disorder in a human subject.

In some embodiments, dual prime editing involves using two different PEgRNAs each complexed with a prime editor, wherein each of the two PEgRNAs comprises a spacer complementary or substantially complementary to a separate search target sequence. In some embodiments, each of the two PEgRNAs anneals with a separate search target sequence through its spacer. Accordingly, references to a “PAM strand”, a “non-PAM strand”, a “target strand’, a “non-target strand”, an “edit strand” or a “non-edit strand” are relative in the context of a specific PEgRNA, e.g., one of the two PEgRNAs in dual prime editing.

In some embodiments, dual prime editing involves two PEgRNAs, different from one another, each complexed with a prime editor. In some embodiments, each of the two PEgRNAs comprises a region of complementarity to a distinct search target sequence of the target DNA, wherein the two distinct search target sequences are on the two complementary strands of the target DNA. The terms “region”, “portion”, and “segment” are used interchangeably to refer to a proportion of a molecule, for example, a polynucleotide or a polypeptide. For example, a region of a polynucleotide may be 5%, 10%, 15%, 20%, 25%, 30%, 35%, 40%, 45%, 50%, 55%, 60%, 65%, 70%, 75%, 80%, 85%, 90%, 95% or 100% of the polynucleotide. In some embodiments, the two PEgRNAs each can direct a prime editor to initiate the prime editing process on the two complementary strands of the target DNA.

In some embodiments, dual prime editing involves two PEgRNAs each complexed with a prime editor. In some embodiments, a first PEgRNA comprises a first spacer complementary to a first search target sequence on a first strand of a double-stranded target DNA, e.g., a double-stranded target gene. In the context of the first PERNA, the first strand of the double-stranded target DNA may be referred to as a first target strand, and the complementary strand referred to as the first PAM strand.

In some embodiments, a second PERNA comprises a second spacer complementary to a second search target sequence on a second strand of the double-stranded target DNA. In some embodiments, the first strand and the second strand of the double-stranded target DNA, e.g., a double-stranded target gene, are complementary to each other. Accordingly, in some embodiments, the second PEgRNA and the first PERNA bind opposite strands of the double-stranded target DNA. In the context of the second PEgRNA, the second strand of the double-stranded target DNA may be referred to as a second target strand, and the complementary strand referred to as the second PAM strand. In some embodiments, the first target strand is the same strand as the second PAM strand of the double-stranded target DNA. In some embodiments, the second target strand is the same strand as the first PAM strand of the double-stranded target DNA.

In some embodiments, the first PEgRNA anneals with the first target strand of the double-stranded target DNA, through the first spacer of the first PEgRNA. In some embodiments, the first PEgRNA complexes with and directs a first prime editor to bind the double-stranded target DNA at the position corresponding to the first search target sequence. In some embodiments, the second PEgRNA anneals with the second search target sequence on the second target strand of the double-stranded target DNA, through a second spacer of the second PERNA. In some embodiments, the second PEgRNA complexes with and directs a second prime editor to bind the double-stranded target DNA at the position corresponding to the second search target sequence. In some embodiments, the first prime editor and the second prime editor are the same. In some embodiments, the first prime editor and the second prime editor are different.

In some embodiments, the first search target sequence recognized by the spacer of the first PEgRNA and the second search target sequence recognized by the spacer of the second PEgRNA have a region of complementarity to each other. In some embodiments, the region of complementarity is 2 to 20 nucleotides in length. In some embodiments, the region of complementarity is 5 to 15 nucleotides in length.

In some embodiments, the first search target sequence recognized by the spacer of the first PEgRNA and the second search target sequence recognized by the spacer of the second PEgRNA do not have a region of complementarity to each other. In some embodiments, the positions of the first and second search target sequences relative to each other may be determined by their positions in the double-stranded target DNA prior to editing. In some embodiments, the positions of the first and second search target sequences relative to each other may be determined by their positions in a reference double-stranded target DNA.

In some embodiments, the first search target sequence is upstream of the second search target sequence. In some embodiments, the first search target sequence is downstream of the second search target sequence. In some embodiments, the 5′ end of the first search target sequence is 2, 3, 4, 5, 6, 7, 8, 9, 10, 11, 12, 13, 14, 15, 16, 17, 18, 19, 20, 21,,22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 base pairs upstream of the 5′ end of the second search target sequence. In some embodiments, the 5′ end of the first search target sequence is 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 base pairs upstream of the 5′ end of the second search target sequence. In some embodiments, the 5′ end of the first search target sequence is 500, 600, 700, 800, 900, 1000, 1100, 1200, 1300, 1400, 1500, 1600, 1700, 1800, 1900, 2000 or more base pairs upstream of the 5′ end of the second search target sequence.

In some embodiments, the 3′ end of the first search target sequence is 2, 3, 4, 5, 6, 7, 8, 9, 10, 11, 12, 13, 14, 15, 16, 17, 18, 19, 20, 21,,22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 base pairs downstream of the 3′ end of the second search target sequence. In some embodiments, the 3′ end of the first search target sequence is 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 base pairs downstream of the 3′ end of the second search target sequence. In some embodiments, the 3′ end of the first search target sequence is 500, 600, 700, 800, 900, 1000, 1100, 1200, 1300, 1400, 1500, 1600, 1700, 1800, 1900, 2000 or more base pairs downstream of the 3′ end of the second search target sequence.

In some embodiments, the bound first prime editor generates a first nick on the first PAM strand of the double-stranded target DNA. In some embodiments, a first PEgRNA comprises a first primer binding site (PBS), also referred to herein as “primer binding site sequence”, that is complementary to the sequence of the first PAM strand of the double-stranded target DNA that is immediately upstream of the first nick site, and can anneal with the sequence of the first strand at a free 3′ end formed at the first nick site. In some embodiments, a first PEgRNA comprises a first primer binding site (PBS) that anneals to a free 3′ end formed at the first nick site and the first prime editor initiates DNA synthesis from the nick site, using the free 3′ end as a primer. In some embodiments, the first prime editor generates a first newly synthesized single-stranded DNA encoded by a first editing template of the first PEgRNA.

In some embodiments, the bound second prime editor generates a second nick on the second PAM strand of the double-stranded target DNA. In some embodiments, the double-stranded target DNA, e.g., a target gene, comprises a double-stranded DNA sequence between the first nick generated by the first prime editor on the second target strand (also referred to as the first PAM strand) and the second nick generated by the second prime editor on the first target strand (also referred to as the second PAM strand), which may be referred to as an inter-nick duplex (IND). In some embodiments, the two strands of an IND are completely complementary to each other. In some embodiments, the two strands of an IND are partially complementary to each other. In some embodiments, the IND is subsequently excised from the double-stranded target DNA, e.g., the target gene.

In some embodiments, the IND is 1, 2, 3, 4, 5, 6, 7, 8, 9, 10, 11, 12, 13, 14, 15, 16, 17, 18, 19, 20 or more base pairs in length. In some embodiments, the IND is up to 5, up to 10, up to 15, up to 20, up to 25, up to 30, up to 40, or up to 50 base pairs in length. In some embodiments, the IND is 1-3000, 1-2500, 1-2000, 1-1500, 1-1000, 1-900, 1-800, 1-700, 1-600, 1-500, 1-400, 1-300, 1-200, 1-100, 1-50, 1-40, 1-30, 1-25, 1-20, 1-15, 1-10, or 1-5 base pairs in length. In some embodiments, the IND is 500-3000, 500-2500, 500-2000, 500-1500, 500-1000, 500-900, 500-800, 500-700, or 500-600 base pairs in length. In some embodiments, the IND is 30-300, 30-250, 30-200, 30-150, 30-100, 30-75, 30-50, 50-200, 50-150, 50-100, 50-75, 75-100, 75-150, 75-200, 75-250, 75-300 base pairs in length. In some embodiments, the IND is 1-3, 1-6, 1-9, 1-12, 1-15, 1-18, 1-21, 1-24, 1-27, 1-30, 1-36, 1-45, 1-60, 1-72, 1-90, 3-6, 3-9, 3-12, 3-15, 3-18, 3-21, 3-24, 3-27, 3-30, 3-36, 3-45, 3-60, 3-72, 3-90, 6-9, 6-12, 6-15, 6-18, 6-21, 6-24, 6-27, 6-30, 6-36, 6-45, 6-60, 6-72, 6-90, 9-12, 9-15, 9-18, 9-21, 9-24, 9-27, 9-30, 9-36, 9-45, 9-60, 9-72, 9-90, 12-15, 12-18, 12-21, 12-24, 12-27, 12-30, 12-36, 12-45, 12-60, 12-72, 12-90, 15-18, 15-21, 15-24, 15-27, 15-30, 15-36, 15-45, 15-60, 15-72, 15-90, 18-21, 18-24, 18-27, 18-30, 18-36, 18-45, 18-60, 18-72, 18-90, 21-24, 21-27, 21-30, 21-36, 21-45, 21-60, 21-72, 21-90, 24-27, 24-30, 24-36, 24-45, 24-60, 24-72, 24-90, 27-30, 27-36, 27-45, 27-60, 27-72, 27-90, 30-36, 30-45, 30-60, 30-72, 30-90, 45-60, 45-72, 60-72, 60-90, or 72-90 base pairs in length. In some embodiments, the IND is 1-3000, 1-2500, 1-2000, 1-1500, 1-1000, 1-900, 1-800, 1-700, 1-600, 1-500, 1-400, 1-300, 1-200, 1-100, 1-50, 1-40, 1-30, 1-25, 1-20, 1-15, 1-10, 1-5, 500-3000, 500-2500, 500-2000, 500-1500, 500-1000, 500-900, 500-800, 500-700, 500-600, 30-300, 30-250, 30-200, 30-150, 30-100, 30-75, 30-50, 50-200, 50-150, 50-100, 50-75, 75-100, 75-150, 75-200, 75-250, or 75-300 base pairs in length. In some embodiments, the IND is 1-3, 1-6, 1-9, 1-12, 1-15, 1-18, 1-21, 1-24, 1-27, 1-30, 1-36, 1-45, 1-60, 1-72, 1-90, 3-6, 3-9, 3-12, 3-15, 3-18, 3-21, 3-24, 3-27, 3-30, 3-36, 3-45, 3-60, 3-72, 3-90, 6-9, 6-12, 6-15, 6-18, 6-21, 6-24, 6-27, 6-30, 6-36, 6-45, 6-60, 6-72, 6-90, 9-12, 9-15, 9-18, 9-21, 9-24, 9-27, 9-30, 9-36, 9-45, 9-60, 9-72, 9-90, 12-15, 12-18, 12-21, 12-24, 12-27, 12-30, 12-36, 12-45, 12-60, 12-72, 12-90, 15-18, 15-21, 15-24, 15-27, 15-30, 15-36, 15-45, 15-60, 15-72, 15-90, 18-21, 18-24, 18-27, 18-30, 18-36, 18-45, 18-60, 18-72, 18-90, 21-24, 21-27, 21-30, 21-36, 21-45, 21-60, 21-72, 21-90, 24-27, 24-30, 24-36, 24-45, 24-60, 24-72, 24-90, 27-30, 27-36, 27-45, 27-60, 27-72, 27-90, 30-36, 30-45, 30-60, 30-72, 30-90, 45-60, 45-72, 60-72, 60-90, or 72-90 base pairs in length.

In some embodiments, the double-stranded target DNA is a double-stranded target gene or a part of a double-stranded target gene, and the IND comprises a part of a coding sequence of the target gene. In some embodiments, the IND comprises a part of a non-coding sequence of the target gene. In some embodiments, the IND comprises a part of an exon. In some embodiments, the IND comprises an entire exon. In some embodiments, the IND comprises a part of an intron. In some embodiments, the IND comprises an entire intron. In some embodiments, the IND comprises a 3′ UTR sequence of the target gene. In some embodiments, the IND comprises a 5′ UTR sequence of the target gene. In some embodiments, the IND comprises a whole or a part of an ORF of the target gene. In some embodiments, the IND comprises both coding and non-coding sequences of the target gene. In some embodiments, the IND comprises both intron and exon sequences of the target gene. For example, in some embodiments, the IND comprises the sequence of an exon flanked by an intronic sequence at the 5′ end, the 3′ end, or both ends. In some embodiments, the IND comprises one or more exons and intervening introns. In some embodiments, the IND comprises two or more exons and intervening introns. In some embodiments, the IND comprises all of the coding regions of a target gene, regulatory sequences of a target gene, or the entire target gene comprising its exons, introns and regulatory sequences. In some embodiments, the double-stranded DNA comprises a gene or a part of a gene, and the IND comprises one or more mutations compared to a wild-type reference sequence of the same gene. In some embodiments, the one or more mutations are associated with a disease.

In some embodiments, a first PEgRNA comprises a first primer binding site (PBS) that is complementary to a free 3′ end of the second strand of the double-stranded target DNA formed at the first nick site. In some embodiments, the first PBS anneals with the free 3′ end formed at the first nick site, and the first prime editor initiates DNA synthesis from the first nick site, using the free 3′ end at the first nick site as a primer. In some embodiments, the first prime editor synthesizes a first new single-stranded DNA encoded by the first editing template of the first PEgRNA. In some embodiments, the second PEgRNA comprises a second PBS that is complementary to a free 3′ end of the first strand of the double-stranded target DNA formed at the second nick site. In some embodiments, the second PBS anneals with the free 3′ end formed at the second nick site, and the second prime editor initiates DNA synthesis from the nick site, using the free 3′ end at the second nick site as a primer. In some embodiments, the second prime editor synthesizes a second newly synthesized single-stranded DNA encoded by a second editing template of the second PERNA.

In some embodiments, through DNA repair, the sequence of the first newly synthesized single-stranded DNA encoded by the first editing template and/or the sequence of the second newly synthesized single-stranded DNA encoded by the second editing template is incorporated into the double-stranded target DNA, e.g., a target gene, thereby incorporating one or more intended nucleotide edits in the double-stranded target DNA, e.g., the target gene.

As used herein, a “nucleotide edit” or an “intended nucleotide edit” refers to a specified edit of a double-stranded target DNA. A nucleotide edit or intended nucleotide edit refers to a (i) deletion of one or more contiguous nucleotides at one specific position, (ii) insertion of one or more contiguous nucleotides at one specific position, (iii) substitution of one or more contiguous nucleotides, or (iv) a combination of contiguous nucleotide substitutions, insertions and/or deletions of two or more contiguous nucleotides at one specific position, or other alterations at one specific position to be incorporated into the sequence of the double-stranded target DNA. An intended nucleotide edit may refer to the edit on an editing template (e.g., a first editing template or a second editing template) as compared to the sequence of the double-stranded target gene, or may refer to the edit encoded by an editing template in the newly synthesized single-stranded DNA that is incorporated in the double-stranded target DNA, e.g., the TRAC gene, as compared to endogenous sequence of the double-stranded target DNA, e.g., the TRAC gene. In some embodiments, an intended nucleotide edit may also refer to the edit that results from incorporation of the newly synthesized DNA encoded by an editing template, or incorporation of the two newly synthesized single-stranded DNA encoded by each of the first PERNA and the second PEgRNA in dual prime editing.

In some embodiments, the sequence of the first newly synthesized single-stranded DNA and/or the sequence of the second newly synthesized single-stranded DNA are incorporated into the double-stranded target DNA, e.g., the target gene. In some embodiments, the first and/or the second newly synthesized single-stranded DNAs comprises one or more intended nucleotide edits compared to the endogenous sequence of the double-stranded target DNA, e.g., the target gene, which are incorporated in the double-stranded target DNA, e.g., the target gene. In some embodiments, the sequence of the first newly synthesized single-stranded DNA encoded by the first editing template is incorporated in the double-stranded target DNA, e.g., the target gene, thereby incorporating one or more intended nucleotide edits in the double-stranded target DNA, e.g., the target gene. In some embodiments, the sequence of the second newly synthesized single-stranded DNA encoded by the second editing template is incorporated in the double-stranded target DNA, e.g., the target gene, thereby incorporating one or more intended nucleotide edits in the double-stranded target DNA, e.g., the target gene. In some embodiments, the sequence of the first newly synthesized single-stranded DNA encoded by the first editing template and the sequence of the second newly synthesized single-stranded DNA encoded by the second editing template are incorporated in the double-stranded target DNA, e.g., the target gene, thereby incorporating one or more intended nucleotide edits in the double-stranded target DNA, e.g., the target gene.

In some embodiments, the intended nucleotide edit comprises an insertion, deletion, nucleotide substitution, inversion, or any combination thereof compared to the endogenous sequence of the double-stranded target DNA, e.g., the target gene. In some embodiments, the intended nucleotide edit comprises 1, 2, 3, 4, 5, 6, 7, 8, 9, 10, 11, 12, 13, 14, 15, 16, 17, 18, 19, 20 or more nucleotide substitutions compared to the endogenous sequence of the double-stranded target DNA, e.g., the target gene. In some embodiments, the intended nucleotide edit comprises up to 5, up to 10, up to 15, up to 20, up to 25, up to 30, up to 40, or up to 50 nucleotide substitutions compared to the endogenous sequence of the double-stranded target DNA, e.g., the target gene. In some embodiments, the intended nucleotide edit comprises 1-50, 1-40, 1-30, 1-25, 1-20, 1-15, 1-10, or 1-5 nucleotide substitutions compared to the endogenous sequence of the double-stranded target DNA, e.g., the target gene. In some embodiments, the intended nucleotide edit comprises 3-50, 3-40, 3-30, 3-25, 3-20, 3-15, 3-10, or 3-5 nucleotide substitutions compared to the endogenous sequence of the double-stranded target DNA, e.g., the target gene. In some embodiments, the intended nucleotide edit comprises 5-50, 5-40, 5-30, 5-25, 5-20, 5-15, or 5-10 nucleotide substitutions compared to the endogenous sequence of the double-stranded target DNA, e.g., the target gene.

In some embodiments, the intended nucleotide edit comprises 1, 2, 3, 4, 5, 6, 7, 8, 9, 10, 11, 12, 13, 14, 15, 16, 17, 18, 19, 20 or more nucleotide insertions compared to the endogenous sequence of the double-stranded target DNA, e.g., the target gene. In some embodiments, the intended nucleotide edit comprises up to 5, up to 10, up to 15, up to 20, up to 25, up to 30, up to 40, or up to 50 nucleotide insertions compared to the endogenous sequence of the double-stranded target DNA, e.g., the target gene. In some embodiments, the intended nucleotide edit comprises single nucleotide insertions at 1, 2, 3, 4, 5, 6, 7, 8, 9, 10, 11, 12, 13, 14, 15, 16, 17, 18, 19, 20 or more sites in the double-stranded target DNA compared to the endogenous sequence of the double-stranded target DNA, e.g., the target gene. In some embodiments, the intended nucleotide edit comprises nucleotide insertions of greater than one nucleotide at each site in the double-stranded target DNA compared to the endogenous sequence of the double-stranded target DNA, e.g., the target gene. As used herein, “site” refers to a specific position in the sequence of a target DNA, e.g., a target gene. In some embodiments, the specific position in the sequence of the double-stranded target DNA, e.g., a target gene, can be referred to by specific positions in a reference sequence, e.g., a wild-type gene sequence. In some embodiments, a nucleotide insertion at position x refers to insertion of one or more nucleotides between position x and position x+1 as set forth by numbering in a reference sequence. In some embodiments, a nucleotide deletion at position x refers to deletion of the specific nucleotide at position x as set forth by numbering in a reference sequence. In some embodiments, a nucleotide deletion of positions x to x+n refers to deletion of the specific nucleotides starting at nucleotide x to nucleotide x+n, including nucleotide x and nucleotide x+n, as set forth by numbering in a reference sequence. In some embodiments, a nucleotide inversion of positions x to x+n refers to inversion of the specific nucleotides starting at nucleotide x to nucleotide x+n, including nucleotide x and nucleotide x+n, as set forth by numbering in a reference sequence.

In some embodiments, the intended nucleotide edit comprises nucleotide insertions of greater than one nucleotide at 1, 2, 3, 4, 5, 6, 7, 8, 9, 10, 11, 12, 13, 14, 15, 16, 17, 18, 19, 20 or more sites in the double-stranded target DNA compared to the endogenous sequence of the double-stranded target DNA, e.g., the target gene. In some embodiments, the intended nucleotide edit comprises 1-3000, 1-2500, 1-2000, 1-1500, 1-1000, 1-900, 1-800, 1-700, 1-600, 1-500, 1-400, 1-300, 1-200, 1-100, 1-50, 1-40, 1-30, 1-25, 1-20, 1-15, 1-10, or 1-5 nucleotide insertions compared to the endogenous sequence of the double-stranded target DNA, e.g., the target gene. In some embodiments, the intended nucleotide edit comprises 500-3000, 500-2500, 500-2000, 500-1500, 500-1000, 500-900, 500-800, 500-700, or 500-600 nucleotide insertions compared to the endogenous sequence of the double-stranded target DNA, e.g., the target gene. In some embodiments, the intended nucleotide edit comprises 30-300, 30-250, 30-200, 30-150, 30-100, 30-75, 30-50, 50-200, 50-150, 50-100, 50-75, 75-100, 75-150, 75-200, 75-250, or 75-300 nucleotide insertions compared to the endogenous sequence of the double-stranded target DNA, e.g., the target gene. In some embodiments, the intended nucleotide edit comprises nucleotide insertions of 1-3000, 1-2500, 1-2000, 1-1500, 1-1000, 1-900, 1-800, 1-700, 1-600, 1-500, 1-400, 1-300, 1-200, 1-100, 1-50, 1-40, 1-30, 1-25, 1-20, 1-15, 1-10, 1-5, 500-3000, 500-2500, 500-2000, 500-1500, 500-1000, 500-900, 500-800, 500-700, 500-600, 30-300, 30-250, 30-200, 30-150, 30-100, 30-75, 30-50, 50-200, 50-150, 50-100, 50-75, 75-100, 75-150, 75-200, 75-250, or 75-300 nucleotides at each site in the double-stranded target DNA compared to the endogenous sequence of the double-stranded target DNA, e.g., the target gene.

In some embodiments, the intended nucleotide edit comprises 1, 2, 3, 4, 5, 6, 7, 8, 9, 10, 11, 12, 13, 14, 15, 16, 17, 18, 19, 20 or more nucleotide deletions compared to the endogenous sequence of the double-stranded target DNA, e.g., the target gene. In some embodiments, the intended nucleotide edit comprises up to 5, up to 10, up to 15, up to 20, up to 25, up to 30, up to 40, or up to 50 nucleotide deletions compared to the endogenous sequence of the double-stranded target DNA, e.g., the target gene. In some embodiments, the intended nucleotide edit comprises single nucleotide deletions at 1, 2, 3, 4, 5, 6, 7, 8, 9, 10, 11, 12, 13, 14, 15, 16, 17, 18, 19, 20 or more sites in the double-stranded target DNA compared to the endogenous sequence of the double-stranded target DNA, e.g., the target gene. In some embodiments, the intended nucleotide edit comprises nucleotide deletions of greater than one nucleotide at each site in the double-stranded target DNA compared to the endogenous sequence of the double-stranded target DNA, e.g., the target gene. In some embodiments, the intended nucleotide edit comprises nucleotide deletions of greater than one nucleotide at 1, 2, 3, 4, 5, 6, 7, 8, 9, 10, 11, 12, 13, 14, 15, 16, 17, 18, 19, 20 or more sites in the double-stranded target DNA compared to the endogenous sequence of the double-stranded target DNA, e.g., the target gene. In some embodiments, the intended nucleotide edit comprises 1-3000, 1-2500, 1-2000, 1-1500, 1-1000, 1-900, 1-800, 1-700, 1-600, 1-500, 1-400, 1-300, 1-200, 1-100, 1-50, 1-40, 1-30, 1-25, 1-20, 1-15, 1-10, or 1-5 nucleotide deletions compared to the endogenous sequence of the double-stranded target DNA, e.g., the target gene. In some embodiments, the intended nucleotide edit comprises 500-3000, 500-2500, 500-2000, 500-1500, 500-1000, 500-900, 500-800, 500-700, or 500-600 nucleotide deletions compared to the endogenous sequence of the double-stranded target DNA, e.g., the target gene. In some embodiments, the intended nucleotide edit comprises 30-300, 30-250, 30-200, 30-150, 30-100, 30-75, 30-50, 50-200, 50-150, 50-100, 50-75, 75-100, 75-150, 75-200, 75-250, 75-300 nucleotide deletions compared to the endogenous sequence of the double-stranded target DNA, e.g., the target gene.

In some embodiments, the intended nucleotide edit comprises 1-3, 1-6, 1-9, 1-12, 1-15, 1-18, 1-21, 1-24, 1-27, 1-30, 1-36, 1-45, 1-60, 1-72, 1-90, 3-6, 3-9, 3-12, 3-15, 3-18, 3-21, 3-24, 3-27, 3-30, 3-36, 3-45, 3-60, 3-72, 3-90, 6-9, 6-12, 6-15, 6-18, 6-21, 6-24, 6-27, 6-30, 6-36, 6-45, 6-60, 6-72, 6-90, 9-12, 9-15, 9-18, 9-21, 9-24, 9-27, 9-30, 9-36, 9-45, 9-60, 9-72, 9-90, 12-15, 12-18, 12-21, 12-24, 12-27, 12-30, 12-36, 12-45, 12-60, 12-72, 12-90, 15-18, 15-21, 15-24, 15-27, 15-30, 15-36, 15-45, 15-60, 15-72, 15-90, 18-21, 18-24, 18-27, 18-30, 18-36, 18-45, 18-60, 18-72, 18-90, 21-24, 21-27, 21-30, 21-36, 21-45, 21-60, 21-72, 21-90, 24-27, 24-30, 24-36, 24-45, 24-60, 24-72, 24-90, 27-30, 27-36, 27-45, 27-60, 27-72, 27-90, 30-36, 30-45, 30-60, 30-72, 30-90, 45-60, 45-72, 60-72, 60-90, or 72-90 nucleotide deletions compared to the endogenous sequence of the double-stranded target DNA, e.g., the target gene. In some embodiments, the intended nucleotide edit comprises nucleotide deletions of 1-3000, 1-2500, 1-2000, 1-1500, 1-1000, 1-900, 1-800, 1-700, 1-600, 1-500, 1-400, 1-300, 1-200, 1-100, 1-50, 1-40, 1-30, 1-25, 1-20, 1-15, 1-10, 1-5, 500-3000, 500-2500, 500-2000, 500-1500, 500-1000, 500-900, 500-800, 500-700, 500-600, 30-300, 30-250, 30-200, 30-150, 30-100, 30-75, 30-50, 50-200, 50-150, 50-100, 50-75, 75-100, 75-150, 75-200, 75-250, or 75-300 nucleotides at each site in the double-stranded target DNA compared to the endogenous sequence of the double-stranded target DNA, e.g., the target gene. In some embodiments, the intended nucleotide edit comprises nucleotide deletions of 1-3, 1-6, 1-9, 1-12, 1-15, 1-18, 1-21, 1-24, 1-27, 1-30, 1-36, 1-45, 1-60, 1-72, 1-90, 3-6, 3-9, 3-12, 3-15, 3-18, 3-21, 3-24, 3-27, 3-30, 3-36, 3-45, 3-60, 3-72, 3-90, 6-9, 6-12, 6-15, 6-18, 6-21, 6-24, 6-27, 6-30, 6-36, 6-45, 6-60, 6-72, 6-90, 9-12, 9-15, 9-18, 9-21, 9-24, 9-27, 9-30, 9-36, 9-45, 9-60, 9-72, 9-90, 12-15, 12-18, 12-21, 12-24, 12-27, 12-30, 12-36, 12-45, 12-60, 12-72, 12-90, 15-18, 15-21, 15-24, 15-27, 15-30, 15-36, 15-45, 15-60, 15-72, 15-90, 18-21, 18-24, 18-27, 18-30, 18-36, 18-45, 18-60, 18-72, 18-90, 21-24, 21-27, 21-30, 21-36, 21-45, 21-60, 21-72, 21-90, 24-27, 24-30, 24-36, 24-45, 24-60, 24-72, 24-90, 27-30, 27-36, 27-45, 27-60, 27-72, 27-90, 30-36, 30-45, 30-60, 30-72, 30-90, 45-60, 45-72, 60-72, 60-90, or 72-90 nucleotides at each site in the double-stranded target DNA compared to the endogenous sequence of the double-stranded target DNA, e.g., the target gene.

In some embodiments, the intended nucleotide edits, e.g., nucleotide substitutions, insertions, or deletions, are in consecutive or contiguous nucleotides in the double-stranded target DNA sequence compared to the endogenous sequence of the double-stranded target DNA, e.g., the target gene. In some embodiments, the intended nucleotide edits, e.g., nucleotide substitutions, insertions, or deletions are in non-consecutive or non-contiguous nucleotides in the double-stranded target DNA sequence compared to the endogenous sequence of the double-stranded target DNA, e.g., the target gene.

In some embodiments, the intended nucleotide edit comprises an inversion as compared to the endogenous sequence of the double-stranded target DNA, e.g., the target gene. In some embodiments, a segment of 2, 3, 4, 5, 6, 7, 8, 9, 10, 20, 30, 40, 50, 60, 70, 80, 90, 100, 110, 120, 130, 140, 150, 200, 250, 300 or more nucleotides of the endogenous sequence of the double-stranded target DNA is inverted. In some embodiments, a segment of 1-50, 1-40, 1-30, 1-25, 1-20, 1-15, 1-10, or 1-5 nucleotides of the endogenous sequence of the double-stranded target DNA is inverted. In some embodiments, a segment of 3-50, 3-40, 3-30, 3-25, 3-20, 3-15, 3-10, 3-5, 5-50, 5-40, 5-30, 5-25, 5-20, 5-15, or 5-10 nucleotides of the endogenous sequence of the double-stranded target DNA is inverted.

In some embodiments, the intended nucleotide edit comprises more than one nucleotide edit in the double-stranded target DNA sequence compared to the endogenous sequence of the double-stranded target DNA, e.g., the target gene. In some embodiments, the intended nucleotide edit comprises a combination of one or more of nucleotide substitutions, one or more of nucleotide insertions, one or more of nucleotide deletions and one or more of nucleotide inversions compared to the endogenous sequence of the double-stranded target DNA, e.g., the target gene. In some embodiments, the intended nucleotide edit comprises one or more nucleotide substitutions and one or more nucleotide insertions. In some embodiments, the intended nucleotide edit comprises one or more nucleotide substitutions and one or more nucleotide deletions. In some embodiments, the intended nucleotide edit comprises one or more nucleotide substitutions and one or more nucleotide inversions. In some embodiments, the intended nucleotide edit comprises one or more nucleotide insertions and one or more nucleotide deletions. In some embodiments, the intended nucleotide edit comprises one or more nucleotide insertions and one or more nucleotide inversions. In some embodiments, the intended nucleotide edit comprises one or more nucleotide deletions and one or more nucleotide inversions. In some embodiments, the intended nucleotide edit comprises one or more nucleotide substitutions, one or more nucleotide insertions and one or more nucleotide deletions. In some embodiments, the intended nucleotide edit comprises one or more nucleotide substitutions, one or more nucleotide insertions and one or more nucleotide inversions. In some embodiments, the intended nucleotide edit comprises one or more nucleotide substitutions, one or more nucleotide deletions and one or more nucleotide inversions. In some embodiments, the intended nucleotide edit comprises one or more nucleotide insertions, one or more nucleotide deletions and one or more nucleotide inversions. In some embodiments, the intended nucleotide edit comprises one or more nucleotide substitutions, one or more nucleotide insertions, one or more nucleotide deletions and one or more nucleotide inversions.

In some embodiments, the first newly synthesized single-stranded DNA and the second newly synthesized single-stranded DNA have a region of complementarity to each other. In some embodiments, the first newly synthesized single-stranded DNA has a region of complementarity to an endogenous sequence of the double-stranded target DNA, e.g., the target gene, adjacent to or near a nick site. In some embodiments, the first newly synthesized single-stranded DNA has a region of complementarity to an endogenous sequence of the double-stranded target DNA, e.g., the target gene, on the first strand adjacent to the second nick site. In some embodiments, the first newly synthesized single-stranded DNA has a region of complementarity to an endogenous sequence of the double-stranded target DNA, e.g., the target gene, on the first strand adjacent to and downstream of the second nick site. In some embodiments, the first newly synthesized single-stranded DNA has a region of identity to an endogenous sequence of the double-stranded target on the second strand adjacent to and downstream of the first nick site. In some embodiments, the first newly synthesized single-stranded DNA has a region of identity to an endogenous sequence of the double-stranded target on the second strand adjacent to and upstream of the second nick site.

In some embodiments, the second newly synthesized single-stranded DNA has a region of complementarity to an endogenous sequence of the double-stranded target DNA, e.g., the target gene, adjacent to or near a nick site. In some embodiments, the second newly synthesized single-stranded DNA has a region of complementarity to an endogenous sequence of the double-stranded target DNA, e.g., the target gene, on the second strand adjacent to the first nick site. In some embodiments, the second newly synthesized single-stranded DNA has a region of complementarity to an endogenous sequence of the double-stranded target DNA, e.g., the target gene, on the second strand adjacent to and downstream of the first nick site. In some embodiments, the second newly synthesized single-stranded DNA has a region of identity to an endogenous sequence of the double-stranded target on the first strand adjacent to and upstream of the second nick site.

As used herein, reference for positioning in a chromosome or a double-stranded polynucleotide, e.g., a double-stranded target DNA, includes the position on either strand of the two strands, unless otherwise specified. For example, a position of a first nick site may be used refer to the first nick site on the first edit strand and/or the corresponding position on the second edit strand.

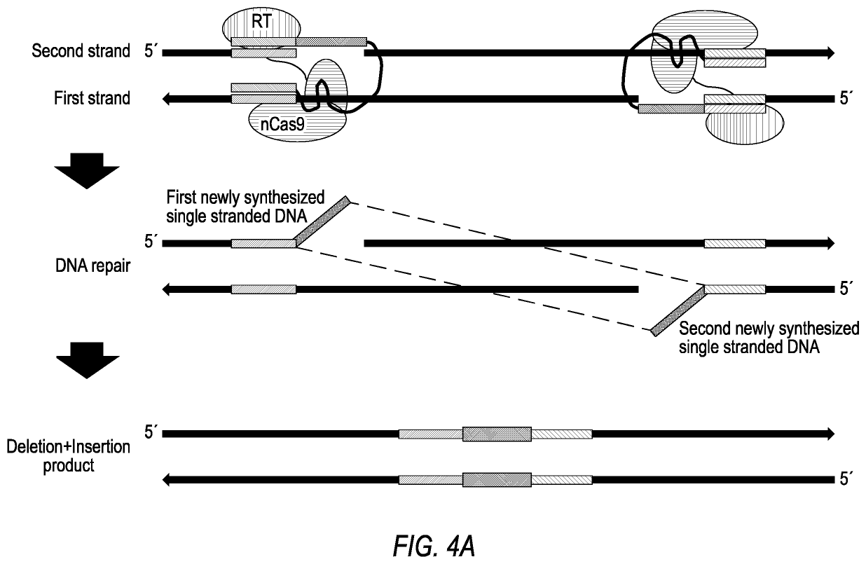

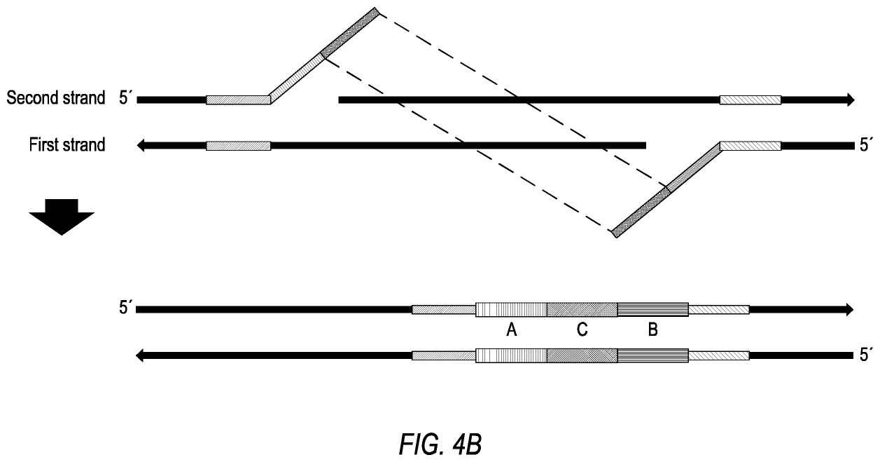

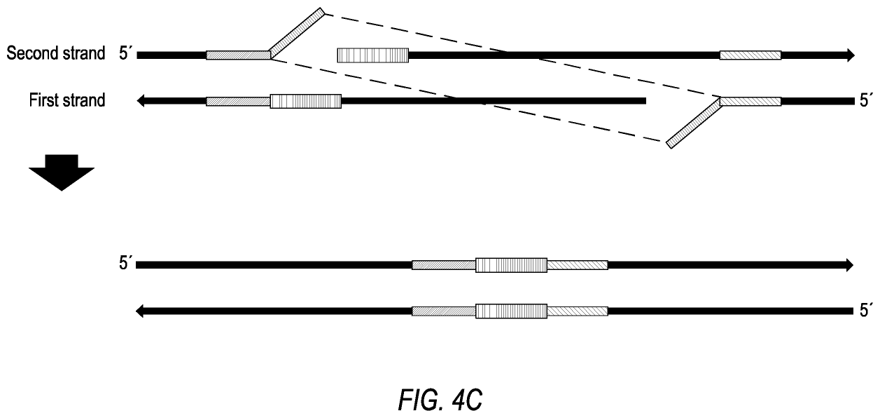

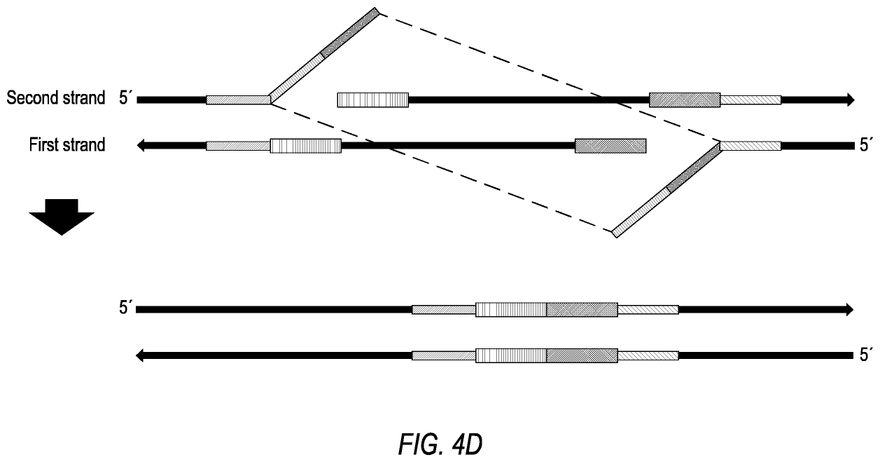

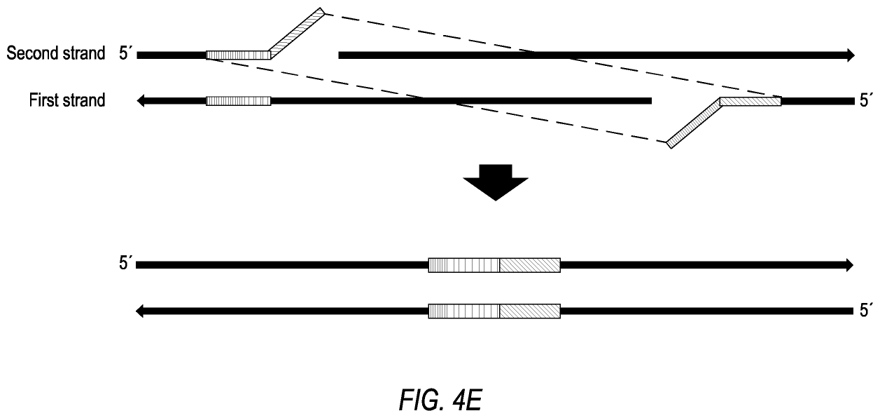

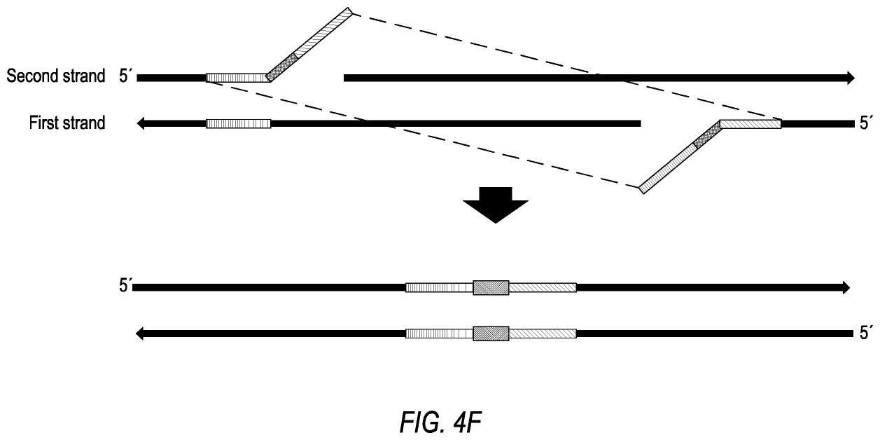

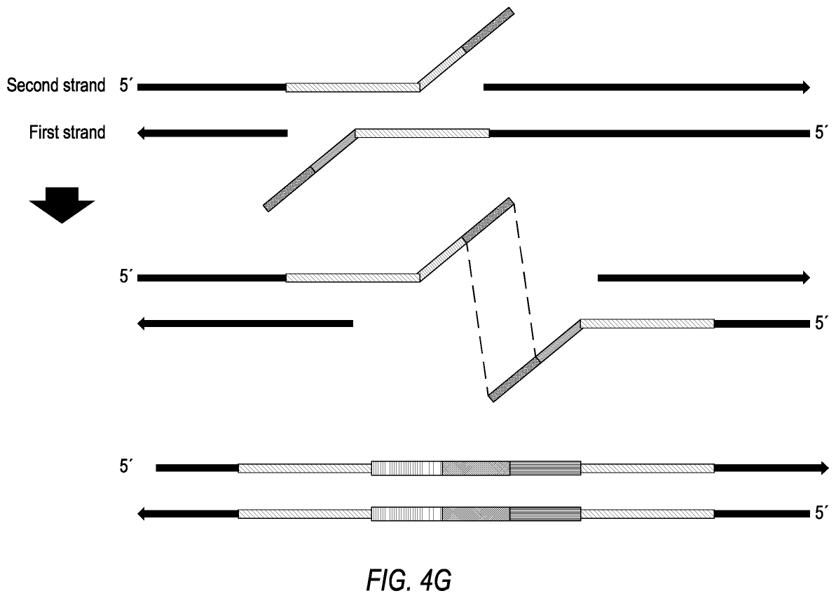

By “upstream” and “downstream” it is intended to define relative positions of at least two regions or sequences in a nucleic acid molecule oriented in a 5′-to-3′ direction. For example, a first sequence is upstream of a second sequence in a DNA molecule where the first sequence is positioned 5′ to the second sequence. Accordingly, the second sequence is downstream, that is, 3′, of the first sequence. In the context of dual prime editing of a double-stranded target DNA, reference to upstream or downstream positioning, unless otherwise specified, the 5′ to 3′ direction is based on a reference strand that is the protein encoding strand (also referred to as the sense strand) of the double-stranded target DNA, e.g., the HTT gene, regardless of whether the sequence is in a translated region. In some embodiments, the reference strand is the first edit strand (i.e. the second strand as shown in FIG. 4A). In embodiments wherein two sequences are on different strands of a double-stranded polynucleotide, e.g., the sense strand and the antisense strand of a double-stranded target DNA, the sequence defined as the upstream (or the 5′) sequence and the sequence defined as the downstream (or the 3′) sequence are based on the position of the sequence on the sense strand (e.g., the first edit strand as exemplified in FIG. 4A) compared to the position of the complementary sequence of the sequence on the antisense strand.

In some embodiments, each of the first newly synthesized single-stranded DNA and the second newly synthesized single-stranded DNA has a region of complementarity to an endogenous sequence of the double-stranded target DNA, e.g., the target gene, adjacent to or near a nick site. In some embodiments, each of the first newly synthesized single-stranded DNA and the second newly synthesized single-stranded DNA has a region of identity to an endogenous sequence of the double-stranded target DNA, e.g., the target gene, adjacent to or near a nick site. In some embodiments, the first newly synthesized single-stranded DNA has a region of complementarity to an endogenous sequence of the double-stranded target DNA, e.g., the target gene, on the first strand adjacent to and downstream of the second nick site, and the second newly synthesized single-stranded DNA has a region of complementarity to an endogenous sequence of the double-stranded target DNA, e.g., the target gene, on the second strand adjacent to and upstream of the first nick site. In some embodiments, the first newly synthesized single-stranded DNA has a region of identity to an endogenous sequence of the double-stranded target DNA on the second strand adjacent to and downstream of the first nick site and/or a region of identity to an endogenous sequence of the double-stranded target DNA on the second strand adjacent to and upstream of the second nick site, and the second newly synthesized single-stranded DNA has a region of identity to an endogenous sequence of the double-stranded target DNA on the first strand adjacent to and downstream of the first nick site and/or a region of identity to an endogenous sequence of the double-stranded target DNA on the second strand adjacent to and upstream of the second nick site.

In some embodiments, the first newly synthesized single-stranded DNA encoded by the first editing template and the second newly synthesized single-stranded DNA encoded by the second editing template have a region of complementarity to each other. The complementary region between the first newly synthesized single-stranded DNA and the second newly synthesized single-stranded DNA may be referred to as an overlap duplex (OD).

In some embodiments, the first newly synthesized single-stranded DNA encoded by the first editing template and the second newly synthesized single-stranded DNA encoded by the second editing template are complementary or substantially complementary to each other. In some embodiments, the OD is incorporated in the double-stranded target DNA, e.g., the target gene, thereby incorporating one or more intended nucleotide edits encoded by the first editing template and the second editing template into the double-stranded target DNA, e.g., the target gene. In some embodiments, the OD replaces all or a portion of the IND, thereby incorporating one or more intended nucleotide edits in the double-stranded target DNA, e.g., the target gene. In some embodiments, the IND is excised or degraded, and the OD is incorporated at the place of the IND excision, followed by ligation of the nicks on both strands of the double-stranded target DNA, e.g., the target gene, thereby incorporating the one or more intended nucleotide edits in the double-stranded target DNA. In some embodiments, the sequence of the OD comprises partial identity compared to the sequence of the IND. In some embodiments, the sequence of the OD comprises no identity compared to the sequence of the IND. In some embodiments, the sequence of the OD comprises a sequence exogenous to the double-stranded target DNA. In some embodiments, incorporation of the OD does not alter the reading frame of the double-stranded target DNA.

In some embodiments, the first editing template and the second editing template comprise a region of complementarity or substantial complementarity to each other, and do not have complementarity to either strand of the double-stranded target DNA, e.g., the target gene. Accordingly, in some embodiments, the first newly synthesized single-stranded DNA encoded by the first editing template and the second newly synthesized single-stranded DNA encoded by the second editing template can anneal to each other to form an OD that does not have nucleotide sequence identity with the endogenous sequence of double-stranded target DNA, e.g., the target gene. In some embodiments, the sequence of the OD comprises a sequence exogenous to the double-stranded target DNA, e.g., the target gene. In some embodiments, the sequence of the OD consists of a sequence exogenous to the double-stranded target DNA, e.g., the target gene. In some embodiments, the IND is excised, and the OD is incorporated at the place of the IND excision, followed by ligation of the nicks on both strands of the target DNA, thereby incorporating the sequence of the OD in the double-stranded target DNA.

In some embodiments, the OD comprises 1, 2, 3, 4, 5, 6, 7, 8, 9, 10, 11, 12, 13, 14, 15, 16, 17, 18, 19, 20, 21, 22, 23, 24, 25, 30, 35, 40, 45, 50 or more contiguous complementary or substantially complementary base pairs. In some embodiments, the OD comprises about 5 to 10, 5 to 15, 5 to 20, 5 to 25, 5 to 30, 5 to 35, 5 to 40, 5 to 45, 5 to 50, 5 to 55, 5 to 60, 5 to 65, 5 to 70, 5 to 75, 5 to 80, 5 to 85, 5 to 90, 5 to 95, 5 to 100, 5 to 110, 5 to 120, 5 to 130, 5 to 140, 5 to 150, 15 to 20, 15 to 25, 15 to 30, 15 to 35, 15 to 40, 15 to 45, 15 to 50, 15 to 55, 15 to 60, 15 to 65, 15 to 70, 15 to 75, 15 to 80, 15 to 85, 15 to 90, 15 to 95, 15 to 100, 15 to 110, 15 to 120, 15 to 130, 15 to 140, 15 to 150, 25 to 30, 25 to 35, 25 to 40, 25 to 45, 25 to 50, 25 to 55, 25 to 60, 25 to 65, 25 to 70, 25 to 75, 25 to 80, 25 to 85, 25 to 90, 25 to 95, 25 to 100, 25 to 110, 25 to 120, 25 to 130, 25 to 140, 25 to 150, 35 to 40, 35 to 45, 35 to 50, 35 to 55, 35 to 60, 35 to 65, 35 to 70, 35 to 75, 35 to 80, 35 to 85, 35 to 90, 35 to 95, 35 to 100, 35 to 110, 35 to 120, 35 to 130, 35 to 140, 35 to 150, 45 to 50, 45 to 55, 45 to 60, 45 to 65, 45 to 70, 45 to 75, 45 to 80, 45 to 85, 45 to 90, 45 to 95, 45 to 100, 45 to 110, 45 to 120, 45 to 130, 45 to 140, o45 to 150, 55 to 60, 55 to 65, 55 to 70, 55 to 75, 55 to 80, 55 to 85, 55 to 90, 55 to 95, 55 to 100, 55 to 110, 55 to 120, 55 to 130, 55 to 140, 55 to 150, 65 to 70, 65 to 75, 65 to 80, 65 to 85, 65 to 90, 65 to 95, 65 to 100, 65 to 110, 65 to 120, 65 to 130, 65 to 140, 65 to 150, 75 to 80, 75 to 85, 75 to 90, 75 to 95, 75 to 100, 75 to 110, 75 to 120, 75 to 130, 75 to 140, 75 to 150, 85 to 90, 85 to 95, 85 to 100, 85 to 110, 85 to 120, 85 to 130, 85 to 140, 85 to 150, 95 to 100, 95 to 110, 95 to 120, 95 to 130, 95 to 140, 95 to 150, 105 to 110, 105 to 120, 105 to 130, 105 to 140, 105 to 150, 115 to 120, 115 to 130, 115 to 140, 115 to 150, 125 to 130, 125 to 140, 125 to 150, 135 to 140, 135 to 150, or 145 to 150 contiguous complementary or substantially complementary base pairs. In some embodiments, the OD comprise 10, 11, 12, 13, 14, 15, 16, 17, 18, 19, 20, 21, 22, 23, 24 or 25 contiguous complementary or substantially complementary base pairs. In some embodiments, the OD comprise 30, 35, 40, 50, 60, 70, 80, 90, or 100 contiguous complementary or substantially complementary base pairs. In some embodiments, the OD comprise no greater than 20, 25, 30, 35, 40, 50, 60, 70, 80, 90, or 100 contiguous complementary or substantially complementary base pairs. In some embodiments, the OD comprises a sufficient number of contiguous complementary base pairs to form a sufficiently stable duplex for replacement of the IND. In some embodiments, the OD comprises at least 10 contiguous complementary or substantially complementary base pairs. In some embodiments, the OD comprises at least 15 contiguous complementary or substantially complementary base pairs. In some embodiments, the OD comprises about 20 contiguous complementary or substantially complementary base pairs.

In some embodiments, the OD replaces the IND of a target DNA, wherein the double-stranded target DNA is an entire target gene or is part of a target gene. In some embodiments, the OD replaces part of an exon or an entire exon, part of an intron or an entire intron, one or more exons and intervening introns, all of the coding regions of a target gene, regulatory sequences of a target gene, or the entire target gene comprising its exons, introns and regulatory sequences. In some embodiments, the OD comprises a region of identity to an endogenous sequence of the double-stranded target DNA. In some embodiments, the OD does not have sequence identity to an endogenous sequence of the double-stranded target DNA. In some embodiments, the OD is exogenous to the double-stranded target DNA, e.g., the target gene.

In some embodiments, the OD has a biological function or encodes a polypeptide having a biological function, or a portion thereof. In some embodiments, the OD comprises an expression cassette. In some embodiments, the OD comprises a nucleotide sequence that encodes an expression tag, for example, an affinity tag, a His tag, a V5 tag, or a FLAG tag. In some embodiments, the OD comprises a nucleotide sequence that encodes a His tag. In some embodiments, the OD comprises a nucleotide sequence that encodes a FLAG tag. In some embodiments, the OD comprises a nucleotide sequence that encodes an attB or an attP sequence. In some embodiments, the OD comprises a nucleotide sequence that encodes a reporter protein, for example, a green fluorescence protein, a blue fluorescence protein, a cyan fluorescence protein, a yellow fluorescence protein, an auto fluorescent protein, or a luciferase. In some embodiments, the OD comprises a recognition site of an enzyme, for example, a recombinase recognition sequence. In some embodiments, the OD comprises nucleotide sequence that encodes a selectable marker, for example, an antibiotic resistance marker. In some embodiments, the OD comprises a regulatory sequence, for example, a promoter, an enhancer, or an insulator. In some embodiments, the OD comprises a trackable sequence, for example, a barcode. In some embodiments, replacement of the IND by the OD decreases or abolishes the expression or the function of the target gene (e.g., TRAC gene). In some embodiments, replacement of the IND by the OD results in disruption of the target DNA (e.g., a TRAC gene) and insertion of one or more recombinase recognition sequences encoded by the OD. In some embodiments, the target gene is a disease-associated gene. In some embodiments, the target gene is a monogenic disease-associated gene. In some embodiments, the target gene is a polygenic disease-associated gene. In some embodiments, the target gene is a disease-associated gene containing one or more disease-causing mutations, wherein replacement of the IND by the OD corrects the mutations, thereby restoring or partially restoring the function of the target gene. In some embodiments, the disease-associated gene containing one or more disease-causing mutations is in a human subject in need of treatment. In some embodiments, the target gene is a mutated gene causing a disease or disorder in a human subject, wherein replacement of the IND by the OD corrects the mutated gene, thereby restoring or partially restoring the function of the target gene. In some embodiments, the target gene is a disease-associated gene containing one or more disease-causing mutations, wherein replacement of the IND by the OD modifies the target gene to restore or partially restore the function of the target gene. In some embodiments, the disease-associated gene containing one or more disease-causing mutations is in a human subject in need of treatment. In some embodiments, the target gene is a mutated gene causing a disease or disorder in a human subject, wherein replacement of the IND by the OD modifies the mutated gene to restore or partially restore the function of the target gene. In some embodiments, the target gene is a wildtype gene, e.g., a wildtype TRAC. In some embodiments, replacement of the IND by the OD modifies the target gene to decrease expression or function of the target gene, a mRNA or a protein encoded by the target gene. In some embodiments, the target gene is a TRAC gene. In some embodiments, replacement of the IND by the OD modifies the TRAC gene to decrease a function of the TRAC gene, TRAC mRNA and/or TRAC protein encoded by the TRAC gene. In some embodiments, replacement of the IND by the OD results in disruption of the target gene (e.g., a TRAC gene) and insertion of one or more exogenous sequences, e.g., one or more recombinase recognition sequences into the gene.

In some embodiments, the first newly synthesized single-stranded DNA encoded by the first editing template and the second newly synthesized single-stranded DNA encoded by the second editing template comprises a region of complementarity with each other, and can anneal with each other to form an OD. In some embodiments, the first newly synthesized single-stranded DNA encoded by the first editing template further comprises a region that does not have complementarity with the second newly synthesized single-stranded DNA encoded by the second editing template (see exemplary schematic in FIG. 4B). In some embodiments, the second newly synthesized single-stranded DNA encoded by the second editing template further comprises a region that does not have complementarity with the first newly synthesized single-stranded DNA encoded by the first editing template. Accordingly, in some embodiments, the first newly synthesized single-stranded DNA encoded by the first editing template and the second newly synthesized single-stranded DNA encoded by the second editing template can anneal to each other through the partially complementary sequences to form an OD that is linked to a 5′ overhang and/or a 3′overhang. In some embodiments, the IND is removed, the OD, along with the 5′ overhang and/or the 3′ overhang, is incorporated at the place of the IND excision in the double-stranded target DNA, e.g., the target gene. Through DNA repair, the gaps corresponding to the positions of the 5′ overhang and/or the 3′ overhangs are filled and ligated, thereby incorporating the one or more intended nucleotide edits in the double-stranded target DNA, e.g., the target gene.

Accordingly, in some embodiments, the IND is replaced by the sequence of (A+C), (B+C), or (A+B+C), wherein A is the region, and its complementary strand, of the first newly synthesized single-stranded DNA that is not complementary to the second newly synthesized single-stranded DNA, wherein B is the region, and its complementary strand, of the second newly synthesized single-stranded DNA that is not complementary to the first newly synthesized single-stranded DNA, and wherein C is the OD. The double-stranded sequence of (A+C), (B+C), or (A+B+C) that replaces the IND may be referred to as the “replacement duplex (RD)”.

Accordingly, in some embodiments, the RD comprises the OD. In some embodiments, as exemplified in FIG. 4A, the first editing template and the second editing template are substantially complementary to each other. Accordingly, in some embodiments, the OD comprises the entirety or substantially the entirety of the first newly synthesized single-stranded DNA encoded by the first editing template and the second newly synthesized single-stranded DNA encoded by the second editing template. In some embodiments, the RD consists of the OD. In some embodiments, as exemplified in FIG. 4B, the RD comprises the OD, the non-complementary region of the first newly synthesized DNA compared to the second newly synthesized DNA and complement thereof, and/or the non-complementary region of the second newly synthesized DNA compared to the first newly synthesized DNA and complement thereof.

In some embodiments, the RD comprises 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 60, 70, 80, 90, 100, 110, 120, 130, 140, 150, 160, 170, 180, 190, 200 or more base pairs. In some embodiments, the RD comprises about 5 to 10, 5 to 15, 5 to 20, 5 to 25, 5 to 30, 5 to 35, 5 to 40, 5 to 45, 5 to 50, 5 to 55, 5 to 60, 5 to 65, 5 to 70, 5 to 75, 5 to 80, 5 to 85, 5 to 90, 5 to 95, 5 to 100, 5 to 110, 5 to 120, 5 to 130, 5 to 140, 5 to 150, 5 to 175, 5 to 200, 5 to 225, 5 to 250, 5 to 275, 5 to 300, 5 to 325, 5 to 350, 5 to 375, 5 to 400, 5 to 425, 5 to 450, 5 to 475, 5 to 500, 10 to 15, 10 to 20, 10 to 25, 10 to 30, 10 to 35, 10 to 40, 10 to 45, 10 to 50, 10 to 55, 10 to 60, 10 to 65, 10 to 70, 10 to 75, 10 to 80, 10 to 85, 10 to 90, 10 to 95, 10 to 100, 10 to 110, 10 to 120, 10 to 130, 10 to 140, 10 to 150, 10 to 175, 10 to 200, 10 to 225, 10 to 250, 10 to 275, 10 to 300, 10 to 325, 10 to 350, 10 to 375, 10 to 400, 10 to 425, 10 to 450, 10 to 475, 10 to 500, 15 to 20, 15 to 25, 15 to 30, 15 to 35, 15 to 40, 15 to 45, 15 to 50, 15 to 55, 15 to 60, 15 to 65, 15 to 70, 15 to 75, 15 to 80, 15 to 85, 15 to 90, 15 to 95, 15 to 100, 15 to 110, 15 to 120, 15 to 130, 15 to 140, 15 to 150, 15 to 175, 15 to 200, 15 to 225, 15 to 250, 15 to 275, 15 to 300, 15 to 325, 15 to 350, 15 to 375, 15 to 400, 15 to 425, 15 to 450, 15 to 475, 15 to 500, 20 to 25, 20 to 30, 20 to 35, 20 to 40, 20 to 45, 20 to 50, 20 to 55, 20 to 60, 20 to 65, 20 to 70, 20 to 75, 20 to 80, 20 to 85, 20 to 90, 20 to 95, 20 to 100, 20 to 110, 20 to 120, 20 to 130, 20 to 140, 20 to 150, 20 to 175, 20 to 200, 20 to 225, 20 to 250, 20 to 275, 20 to 300, 20 to 325, 20 to 350, 20 to 375, 20 to 400, 20 to 425, 20 to 450, 20 to 475, 20 to 500, 30 to 35, 30 to 40, 30 to 45, 30 to 50, 30 to 55, 30 to 60, 30 to 65, 30 to 70, 30 to 75, 30 to 80, 30 to 85, 30 to 90, 30 to 95, 30 to 100, 30 to 110, 30 to 120, 30 to 130, 30 to 140, 30 to 150, 30 to 175, 30 to 200, 30 to 225, 30 to 250, 30 to 275, 30 to 300, 30 to 325, 30 to 350, 30 to 375, 30 to 400, 30 to 425, 30 to 450, 30 to 475, 30 to 500, 40 to 45, 40 to 50, 40 to 55, 40 to 60, 40 to 65, 40 to 70, 40 to 75, 40 to 80, 40 to 85, 40 to 90, 40 to 95, 40 to 100, 40 to 110, 40 to 120, 40 to 130, 40 to 140, 40 to 150, 40 to 175, 40 to 200, 40 to 225, 40 to 250, 40 to 275, 40 to 300, 40 to 325, 40 to 350, 40 to 375, 40 to 400, 40 to 425, 40 to 450, 40 to 475, 40 to 500, 50 to 55, 50 to 60, 50 to 65, 50 to 70, 50 to 75, 50 to 80, 50 to 85, 50 to 90, 50 to 95, 50 to 100, 50 to 110, 50 to 120, 50 to 130, 50 to 140, 50 to 150, 50 to 175, 50 to 200, 50 to 225, 50 to 250, 50 to 275, 50 to 300, 50 to 325, 50 to 350, 50 to 375, 50 to 400, 50 to 425, 50 to 450, 50 to 475, 50 to 500, 75 to 80, 75 to 85, 75 to 90, 75 to 95, 75 to 100, 75 to 110, 75 to 120, 75 to 130, 75 to 140, 75 to 150, 75 to 175, 75 to 200, 75 to 225, 75 to 250, 75 to 275, 75 to 300, 75 to 325, 75 to 350, 75 to 375, 75 to 400, 75 to 425, 75 to 450, 75 to 475, 75 to 500, 100 to 110, 100 to 120, 100 to 130, 100 to 140, 100 to 150, 100 to 175, 100 to 200, 100 to 225, 100 to 250, 100 to 275, 100 to 300, 100 to 325, 100 to 350, 100 to 375, 100 to 400, 100 to 425, 100 to 450, 100 to 475, 100 to 500, 125 to 150, 125 to 175, 125 to 200, 125 to 225, 125 to 250, 125 to 275, 125 to 300, 125 to 325, 125 to 350, 125 to 375, 125 to 400, 125 to 425, 125 to 450, 125 to 475, 125 to 500, 150 to 175, 150 to 200, 150 to 225, 150 to 250, 150 to 275, 150 to 300, 150 to 325, 150 to 350, 150 to 375, 150 to 400, 150 to 425, 150 to 450, 150 to 475, 150 to 500, 175 to 200, 175 to 225, 175 to 250, 175 to 275, 175 to 300, 175 to 325, 175 to 350, 175 to 375, 175 to 400, 175 to 425, 175 to 450, 175 to 475, 175 to 500, 200 to 250, 200 to 275, 200 to 300, 200 to 325, 200 to 350, 200 to 375, 200 to 400, 200 to 425, 200 to 450, 200 to 475, 200 to 500, 225 to 250, 225 to 275, 225 to 300, 225 to 325, 225 to 350, 225 to 375, 225 to 400, 225 to 425, 225 to 450, 225 to 475, 225 to 500, 250 to 275, 250 to 300, 275 to 300, 275 to 325, 275 to 350, 275 to 375, 275 to 400, 275 to 425, 275 to 450, 275 to 475, 275 to 500, 300 to 325, 300 to 350, 300 to 375, 300 to 400, 300 to 425, 300 to 450, 300 to 475, 300 to 500, 325 to 350, 325 to 375, 325 to 400, 325 to 425, 325 to 450, 325 to 475, 325 to 500, 350 to 375, 350 to 400, 350 to 425, 350 to 450, 350 to 475, 350 to 500, 375 to 400, 375 to 425, 375 to 450, 375 to 475, 375 to 500, 400 to 425, 400 to 450, 400 to 475, 400 to 500, 425 to 450, 425 to 475, 425 to 500, 450 to 475, 450 to 500, or 475 to 500 base pairs. In some embodiments, the RD comprise 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 base pairs. In some embodiments, the RD comprise at least 30, 35, 40, 50, 60, 70, 80, 90, 100, 110, 120, 130, 140, 150, 160, 170, 180, 190, 200, 210, 220,230, 240, 250, 260, 270, 280, 290, 300, 310, 320, 330, 340, 350, 360, 370, 380, 390, 400, 410, 420, 430, 440, 450, 460, 470, 480, 490, or 500 base pairs. In some embodiments, the RD comprise no greater than 20, 25, 30, 35, 40, 50, 60, 70, 80, 90, 100, 110, 120, 130, 140, 150, 160, 170, 180, 190, 200, 210, 220,230, 240, 250, 260, 270, 280, 290, 300, 310, 320, 330, 340, 350, 360, 370, 380, 390, 400, 410, 420, 430, 440, 450, 460, 470, 480, 490, or 500 base pairs.

In some embodiments, the RD replaces the IND of a target DNA, wherein the IND is an entire target gene or is part of a target gene. In some embodiments, the RD replaces part of an exon or an entire exon, part of an intron or an entire intron, one or more exons and intervening introns, all of the coding regions of a target gene, regulatory sequences of a target gene, or the entire target gene comprising its exons, introns and regulatory sequences, thereby incorporating the one or more intended nucleotide edits compared to the endogenous sequence of the double-stranded target DNA, e.g., the TRAC gene. In some embodiments, the RD comprises a region of identity to an endogenous sequence of the double-stranded target DNA. In some embodiments, the RD does not have sequence identity to an endogenous sequence of the double-stranded target DNA. In some embodiments, the RD is exogenous to the double-stranded target DNA, e.g., the target gene. Accordingly, in some embodiments, the intended nucleotide edit(s) comprises replacement of an endogenous sequence of the double-stranded target DNA, e.g., the TRAC gene, in its entirety, by the sequence of the RD.

In some embodiments, the RD or the OD may comprise a recombinase recognition sequences (RSSs), e.g., a RSS recognized by a Bxbl recombinase, a Cre recombinase, a Pa01 recombinase, a Si74 recoimbinase, a No67 recombinase, a Kp03 recombinase, a Nm60 recombinase, a BceINTa recombinase, a NcytINTd recombinase, a SscINTd recombinase, a SacINTd recombinase, or a recombinase recognition site corresponding to any recombinase disclosed herein. In some embodiments, the RD or the OD may comprise one, two, or more recombinase recognition sites corresponding to a recombinase.

Replacement of the IND by the RD or the OD comprising one or more recombinase sequences with dual prime editing may result in insertion of the one or more recombinase sequences into the target gene, e.g., a TRAC gene. Depending on the number and orientation of the RSSs, they can be used as landing sites for a recombinase-mediated reaction between the RSSs. For example, a single RSS inserted into a target gene, e.g., a TRAC gene, can be used for integration of an exogenous DNA donor sequence via recombination between the inserted RSS and a second RSS within the exogenous supplied DNA donor If two recombinase sites are inserted in adjacent regions of DNA, depending on the orientation of the recombinase sites, these can be used for recombinase-mediated excision or inversion of the intervening sequence, or for recombinase-mediated cassette exchange with exogenous DNA for cargo integration.

In some embodiments, the RD has a biological function or encodes a polypeptide having a biological function. In some embodiments, the RD comprises an expression cassette. In some embodiments, the RD comprises a nucleotide sequence that encodes an expression tag, for example, an affinity tag, a His tag, a V5 tag, or a FLAG tag. In some embodiments, the RD comprises a nucleotide sequence that encodes a His tag. In some embodiments, the RD comprises a nucleotide sequence that encodes a FLAG tag. In some embodiments, the RD comprises a nucleotide sequence that encodes an attB or an attP sequence. In some embodiments, the RD comprises a nucleotide sequence that encodes a reporter protein, for example, a green fluorescence protein, a blue fluorescence protein, a cyan fluorescence protein, a yellow fluorescence protein, an auto fluorescent protein, or a luciferase. In some embodiments, the RD comprises a recognition site of an enzyme, for example, a recombinase recognition sequence. In some embodiments, the RD comprises a nucleotide sequence that encodes a selectable marker, for example, an antibiotic resistance marker. In some embodiments, the RD comprises a regulatory sequence, for example, a promoter, an enhancer, or an insulator. In some embodiments, the RD comprises a trackable sequence, for example, a barcode. In some embodiments, replacement of the IND by the RD restores or partially restores the function of the target gene. In some embodiments, replacement of the IND by the RD decreases or abolishes the function or expression of the target gene. In some embodiments, the target gene is a TRAC gene. In some embodiments, replacement of the IND by the RD decreases the function of the TRAC gene, TRAC mRNA and/or TRAC protein. In some embodiments, the target gene is a disease-associated gene. In some embodiments, the target gene is a monogenic disease-associated gene. In some embodiments, the target gene is a polygenic disease-associated gene. In some embodiments, the target gene is a disease-associated gene containing one or more disease-causing mutations, wherein replacement of the IND by the RD corrects the mutations, thereby restoring or partially restoring the function of the target gene. In some embodiments, the disease-associated gene containing one or more disease-causing mutations is in a human subject in need of treatment. In some embodiments, the target gene is a mutated gene causing a disease or disorder in a human subject, wherein replacement of the IND by the RD corrects the mutated gene, thereby restoring or partially restoring the function of the target gene. In some embodiments, the target gene is a disease-associated gene containing one or more disease-causing mutations, wherein replacement of the IND by the RD modifies the target gene to restore or partially restore the function of the target gene. In some embodiments, the disease-associated gene containing one or more disease-causing mutations is in a human subject in need of treatment. In some embodiments, the target gene is a mutated gene causing a disease or disorder in a human subject, wherein replacement of the IND by the RD modifies the mutated gene to restore or partially restore the function of the target gene.

In some embodiments, the first editing template and the second editing template are partially complementary to each other. As used herein, the first editing template is partially complementary to the second editing template when the first and the second editing templates have complementary or substantially complementary region(s) over part of the length of both editing templates. The partially complementary region(s) in the first editing template and the second editing template can be in any position within the first editing template and the second editing template. Accordingly, in some embodiments, the first newly synthesized single-stranded DNA encoded by the first editing template and the second newly synthesized single-stranded DNA encoded by the second editing template are partially complementary to each other, at any position within the first newly synthesized single-stranded DNA encoded by the first editing template and the second newly synthesized single-stranded DNA encoded by the second editing template. In some embodiments, the first newly synthesized single-stranded DNA comprises a region of complementarity to the second newly synthesized single-stranded DNA, at or near the 3′ end of the first newly synthesized single-stranded DNA. In some embodiments, the first newly synthesized single-stranded DNA comprises a region of complementarity to the second newly synthesized single-stranded DNA, at or near the 5′ end of the first newly synthesized single-stranded DNA. In some embodiments, the first newly synthesized single-stranded DNA comprises a region of complementarity to the second newly synthesized single-stranded DNA, in the middle of the first newly synthesized single-stranded DNA.

In some embodiments, the second newly synthesized single-stranded DNA comprises a region of complementarity to the first newly synthesized single-stranded DNA, at or near the 3′ end of the second newly synthesized single-stranded DNA. In some embodiments, the second newly synthesized single-stranded DNA comprises a region of complementarity to the first newly synthesized single-stranded DNA, at or near the 5′ end of the second newly synthesized single-stranded DNA. In some embodiments, the second newly synthesized single-stranded DNA comprises a region of complementarity to the first newly synthesized single-stranded DNA, in the middle of the second newly synthesized single-stranded DNA.

In some embodiments, the first newly synthesized single-stranded DNA and the second newly synthesized single-stranded DNA each comprises a region of complementarity to each other at the 3′ end of each of the first newly synthesized single-stranded DNA and the second newly synthesized single-stranded DNA.

In some embodiments, the first editing template and the second editing template are of the same length. In some embodiments, the first editing template and the second editing template are of different lengths.

In some embodiments, the first editing template comprises a region that has complementarity or substantial complementarity to the second editing template (the OD encoding region), and further comprises a region that does not have complementarity to the second editing template. In some embodiments, the first editing template comprises a region that has complementarity or substantial complementarity to the second editing template (the OD encoding region), wherein the region is flanked by one or more regions that do not have complementarity to the second editing template. In some embodiments, the entirety of the first editing template has complementarity or substantial complementarity to a region of the second editing template, wherein the second editing template comprises a region that does not have complementarity to the first editing template.

In some embodiments, the first editing template comprises a region that does not have complementarity to the second editing template, wherein the region is about 5 to 10, 5 to 15, 5 to 20, 5 to 25, 5 to 30, 5 to 35, 5 to 40, 5 to 45, 5 to 50, 5 to 55, 5 to 60, 5 to 65, 5 to 70, 5 to 75, 5 to 80, 5 to 85, 5 to 90, 5 to 95, 5 to 100, 5 to 110, 5 to 120, 5 to 130, 5 to 140, 5 to 150, 5 to 175, 5 to 200, 5 to 225, 5 to 250, 5 to 275, 5 to 300, 5 to 325, 5 to 350, 5 to 375, 5 to 400, 5 to 425, 5 to 450, 5 to 475, 5 to 500, 10 to 15, 10 to 20, 10 to 25, 10 to 30, 10 to 35, 10 to 40, 10 to 45, 10 to 50, 10 to 55, 10 to 60, 10 to 65, 10 to 70, 10 to 75, 10 to 80, 10 to 85, 10 to 90, 10 to 95, 10 to 100, 10 to 110, 10 to 120, 10 to 130, 10 to 140, 10 to 150, 10 to 175, 10 to 200, 10 to 225, 10 to 250, 10 to 275, 10 to 300, 10 to 325, 10 to 350, 10 to 375, 10 to 400, 10 to 425, 10 to 450, 10 to 475, 10 to 500, 15 to 20, 15 to 25, 15 to 30, 15 to 35, 15 to 40, 15 to 45, 15 to 50, 15 to 55, 15 to 60, 15 to 65, 15 to 70, 15 to 75, 15 to 80, 15 to 85, 15 to 90, 15 to 95, 15 to 100, 15 to 110, 15 to 120, 15 to 130, 15 to 140, 15 to 150, 15 to 175, 15 to 200, 15 to 225, 15 to 250, 15 to 275, 15 to 300, 15 to 325, 15 to 350, 15 to 375, 15 to 400, 15 to 425, 15 to 450, 15 to 475, 15 to 500, 20 to 25, 20 to 30, 20 to 35, 20 to 40, 20 to 45, 20 to 50, 20 to 55, 20 to 60, 20 to 65, 20 to 70, 20 to 75, 20 to 80, 20 to 85, 20 to 90, 20 to 95, 20 to 100, 20 to 110, 20 to 120, 20 to 130, 20 to 140, 20 to 150, 20 to 175, 20 to 200, 20 to 225, 20 to 250, 20 to 275, 20 to 300, 20 to 325, 20 to 350, 20 to 375, 20 to 400, 20 to 425, 20 to 450, 20 to 475, 20 to 500, 30 to 35, 30 to 40, 30 to 45, 30 to 50, 30 to 55, 30 to 60, 30 to 65, 30 to 70, 30 to 75, 30 to 80, 30 to 85, 30 to 90, 30 to 95, 30 to 100, 30 to 110, 30 to 120, 30 to 130, 30 to 140, 30 to 150, 30 to 175, 30 to 200, 30 to 225, 30 to 250, 30 to 275, 30 to 300, 30 to 325, 30 to 350, 30 to 375, 30 to 400, 30 to 425, 30 to 450, 30 to 475, 30 to 500, 40 to 45, 40 to 50, 40 to 55, 40 to 60, 40 to 65, 40 to 70, 40 to 75, 40 to 80, 40 to 85, 40 to 90, 40 to 95, 40 to 100, 40 to 110, 40 to 120, 40 to 130, 40 to 140, 40 to 150, 40 to 175, 40 to 200, 40 to 225, 40 to 250, 40 to 275, 40 to 300, 40 to 325, 40 to 350, 40 to 375, 40 to 400, 40 to 425, 40 to 450, 40 to 475, 40 to 500, 50 to 55, 50 to 60, 50 to 65, 50 to 70, 50 to 75, 50 to 80, 50 to 85, 50 to 90, 50 to 95, 50 to 100, 50 to 110, 50 to 120, 50 to 130, 50 to 140, 50 to 150, 50 to 175, 50 to 200, 50 to 225, 50 to 250, 50 to 275, 50 to 300, 50 to 325, 50 to 350, 50 to 375, 50 to 400, 50 to 425, 50 to 450, 50 to 475, 50 to 500, 75 to 80, 75 to 85, 75 to 90, 75 to 95, 75 to 100, 75 to 110, 75 to 120, 75 to 130, 75 to 140, 75 to 150, 75 to 175, 75 to 200, 75 to 225, 75 to 250, 75 to 275, 75 to 300, 75 to 325, 75 to 350, 75 to 375, 75 to 400, 75 to 425, 75 to 450, 75 to 475, 75 to 500, 100 to 110, 100 to 120, 100 to 130, 100 to 140, 100 to 150, 100 to 175, 100 to 200, 100 to 225, 100 to 250, 100 to 275, 100 to 300, 100 to 325, 100 to 350, 100 to 375, 100 to 400, 100 to 425, 100 to 450, 100 to 475, 100 to 500, 125 to 150, 125 to 175, 125 to 200, 125 to 225, 125 to 250, 125 to 275, 125 to 300, 125 to 325, 125 to 350, 125 to 375, 125 to 400, 125 to 425, 125 to 450, 125 to 475, 125 to 500, 150 to 175, 150 to 200, 150 to 225, 150 to 250, 150 to 275, 150 to 300, 150 to 325, 150 to 350, 150 to 375, 150 to 400, 150 to 425, 150 to 450, 150 to 475, 150 to 500, 175 to 200, 175 to 225, 175 to 250, 175 to 275, 175 to 300, 175 to 325, 175 to 350, 175 to 375, 175 to 400, 175 to 425, 175 to 450, 175 to 475, 175 to 500, 200 to 250, 200 to 275, 200 to 300, 200 to 325, 200 to 350, 200 to 375, 200 to 400, 200 to 425, 200 to 450, 200 to 475, 200 to 500, 225 to 250, 225 to 275, 225 to 300, 225 to 325, 225 to 350, 225 to 375, 225 to 400, 225 to 425, 225 to 450, 225 to 475, 225 to 500, 250 to 275, 250 to 300, 275 to 300, 275 to 325, 275 to 350, 275 to 375, 275 to 400, 275 to 425, 275 to 450, 275 to 475, 275 to 500, 300 to 325, 300 to 350, 300 to 375, 300 to 400, 300 to 425, 300 to 450, 300 to 475, 300 to 500, 325 to 350, 325 to 375, 325 to 400, 325 to 425, 325 to 450, 325 to 475, 325 to 500, 350 to 375, 350 to 400, 350 to 425, 350 to 450, 350 to 475, 350 to 500, 375 to 400, 375 to 425, 375 to 450, 375 to 475, 375 to 500, 400 to 425, 400 to 450, 400 to 475, 400 to 500, 425 to 450, 425 to 475, 425 to 500, 450 to 475, 450 to 500, or 475 to 500 nucleotides in length. In some embodiments, the first editing template comprises a region that does not have complementarity to the second editing template, wherein the region is about 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 60, 70, 80, 90, 100, 110, 120, 130, 140, 150, 160, 170, 180, 190, 200, 210, 220,230, 240, 250, 260, 270, 280, 290, 300, 310, 320, 330, 340, 350, 360, 370, 380, 390, 400, 410, 420, 430, 440, 450, 460, 470, 480, 490, or 500 or more nucleotides in length.

In some embodiments, the second editing template comprises a region that has complementarity or substantial complementarity to the first editing template, and further comprises a region that does not have complementarity to the first editing template. In some embodiments, the second editing template comprises a region that has complementarity or substantial complementarity to the first editing template, and is flanked by one or more regions that do not have complementarity to the first editing template. The region(s) in the first editing template and the second editing template may have same or different lengths. In some embodiments, the entirety of the second editing template has complementarity or substantial complementarity to a region of the first editing template, wherein the first editing template comprises a region that does not have complementarity to the second editing template.

In some embodiments, the second editing template comprises a region that does not have complementarity to the first editing template, wherein the region is about 5 to 10, 5 to 15, 5 to 20, 5 to 25, 5 to 30, 5 to 35, 5 to 40, 5 to 45, 5 to 50, 5 to 55, 5 to 60, 5 to 65, 5 to 70, 5 to 75, 5 to 80, 5 to 85, 5 to 90, 5 to 95, 5 to 100, 5 to 110, 5 to 120, 5 to 130, 5 to 140, 5 to 150, 5 to 175, 5 to 200, 5 to 225, 5 to 250, 5 to 275, 5 to 300, 5 to 325, 5 to 350, 5 to 375, 5 to 400, 5 to 425, 5 to 450, 5 to 475, 5 to 500, 10 to 15, 10 to 20, 10 to 25, 10 to 30, 10 to 35, 10 to 40, 10 to 45, 10 to 50, 10 to 55, 10 to 60, 10 to 65, 10 to 70, 10 to 75, 10 to 80, 10 to 85, 10 to 90, 10 to 95, 10 to 100, 10 to 110, 10 to 120, 10 to 130, 10 to 140, 10 to 150, 10 to 175, 10 to 200, 10 to 225, 10 to 250, 10 to 275, 10 to 300, 10 to 325, 10 to 350, 10 to 375, 10 to 400, 10 to 425, 10 to 450, 10 to 475, 10 to 500, 15 to 20, 15 to 25, 15 to 30, 15 to 35, 15 to 40, 15 to 45, 15 to 50, 15 to 55, 15 to 60, 15 to 65, 15 to 70, 15 to 75, 15 to 80, 15 to 85, 15 to 90, 15 to 95, 15 to 100, 15 to 110, 15 to 120, 15 to 130, 15 to 140, 15 to 150, 15 to 175, 15 to 200, 15 to 225, 15 to 250, 15 to 275, 15 to 300, 15 to 325, 15 to 350, 15 to 375, 15 to 400, 15 to 425, 15 to 450, 15 to 475, 15 to 500, 20 to 25, 20 to 30, 20 to 35, 20 to 40, 20 to 45, 20 to 50, 20 to 55, 20 to 60, 20 to 65, 20 to 70, 20 to 75, 20 to 80, 20 to 85, 20 to 90, 20 to 95, 20 to 100, 20 to 110, 20 to 120, 20 to 130, 20 to 140, 20 to 150, 20 to 175, 20 to 200, 20 to 225, 20 to 250, 20 to 275, 20 to 300, 20 to 325, 20 to 350, 20 to 375, 20 to 400, 20 to 425, 20 to 450, 20 to 475, 20 to 500, 30 to 35, 30 to 40, 30 to 45, 30 to 50, 30 to 55, 30 to 60, 30 to 65, 30 to 70, 30 to 75, 30 to 80, 30 to 85, 30 to 90, 30 to 95, 30 to 100, 30 to 110, 30 to 120, 30 to 130, 30 to 140, 30 to 150, 30 to 175, 30 to 200, 30 to 225, 30 to 250, 30 to 275, 30 to 300, 30 to 325, 30 to 350, 30 to 375, 30 to 400, 30 to 425, 30 to 450, 30 to 475, 30 to 500, 40 to 45, 40 to 50, 40 to 55, 40 to 60, 40 to 65, 40 to 70, 40 to 75, 40 to 80, 40 to 85, 40 to 90, 40 to 95, 40 to 100, 40 to 110, 40 to 120, 40 to 130, 40 to 140, 40 to 150, 40 to 175, 40 to 200, 40 to 225, 40 to 250, 40 to 275, 40 to 300, 40 to 325, 40 to 350, 40 to 375, 40 to 400, 40 to 425, 40 to 450, 40 to 475, 40 to 500, 50 to 55, 50 to 60, 50 to 65, 50 to 70, 50 to 75, 50 to 80, 50 to 85, 50 to 90, 50 to 95, 50 to 100, 50 to 110, 50 to 120, 50 to 130, 50 to 140, 50 to 150, 50 to 175, 50 to 200, 50 to 225, 50 to 250, 50 to 275, 50 to 300, 50 to 325, 50 to 350, 50 to 375, 50 to 400, 50 to 425, 50 to 450, 50 to 475, 50 to 500, 75 to 80, 75 to 85, 75 to 90, 75 to 95, 75 to 100, 75 to 110, 75 to 120, 75 to 130, 75 to 140, 75 to 150, 75 to 175, 75 to 200, 75 to 225, 75 to 250, 75 to 275, 75 to 300, 75 to 325, 75 to 350, 75 to 375, 75 to 400, 75 to 425, 75 to 450, 75 to 475, 75 to 500, 100 to 110, 100 to 120, 100 to 130, 100 to 140, 100 to 150, 100 to 175, 100 to 200, 100 to 225, 100 to 250, 100 to 275, 100 to 300, 100 to 325, 100 to 350, 100 to 375, 100 to 400, 100 to 425, 100 to 450, 100 to 475, 100 to 500, 125 to 150, 125 to 175, 125 to 200, 125 to 225, 125 to 250, 125 to 275, 125 to 300, 125 to 325, 125 to 350, 125 to 375, 125 to 400, 125 to 425, 125 to 450, 125 to 475, 125 to 500, 150 to 175, 150 to 200, 150 to 225, 150 to 250, 150 to 275, 150 to 300, 150 to 325, 150 to 350, 150 to 375, 150 to 400, 150 to 425, 150 to 450, 150 to 475, 150 to 500, 175 to 200, 175 to 225, 175 to 250, 175 to 275, 175 to 300, 175 to 325, 175 to 350, 175 to 375, 175 to 400, 175 to 425, 175 to 450, 175 to 475, 175 to 500, 200 to 250, 200 to 275, 200 to 300, 200 to 325, 200 to 350, 200 to 375, 200 to 400, 200 to 425, 200 to 450, 200 to 475, 200 to 500, 225 to 250, 225 to 275, 225 to 300, 225 to 325, 225 to 350, 225 to 375, 225 to 400, 225 to 425, 225 to 450, 225 to 475, 225 to 500, 250 to 275, 250 to 300, 275 to 300, 275 to 325, 275 to 350, 275 to 375, 275 to 400, 275 to 425, 275 to 450, 275 to 475, 275 to 500, 300 to 325, 300 to 350, 300 to 375, 300 to 400, 300 to 425, 300 to 450, 300 to 475, 300 to 500, 325 to 350, 325 to 375, 325 to 400, 325 to 425, 325 to 450, 325 to 475, 325 to 500, 350 to 375, 350 to 400, 350 to 425, 350 to 450, 350 to 475, 350 to 500, 375 to 400, 375 to 425, 375 to 450, 375 to 475, 375 to 500, 400 to 425, 400 to 450, 400 to 475, 400 to 500, 425 to 450, 425 to 475, 425 to 500, 450 to 475, 450 to 500, or 475 to 500 nucleotides in length. In some embodiments, the second editing template comprises a region that does not have complementarity to the first editing template, wherein the region is about 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 60, 70, 80, 90, 100, 110, 120, 130, 140, 150, 160, 170, 180, 190, 200, 210, 220,230, 240, 250, 260, 270, 280, 290, 300, 310, 320, 330, 340, 350, 360, 370, 380, 390, 400, 410, 420, 430, 440, 450, 460, 470, 480, 490, or 500 or more nucleotides in length.

In some embodiments, the RD comprises a region (or a subset) of the sequence of the IND. In some embodiments, the RD consists of a region of the sequence of the IND. In some embodiments, the RD comprises one or more intended nucleotide edits compared to the IND. In some embodiments, the RD comprises a region(s) that has substantial sequence identity to the sequence of the IND, wherein the region(s) comprises one or more nucleotide edits compared to the sequence of the IND. For example, the RD may comprise a region that has substantial sequence identity to the sequence of the IND, wherein the region comprises one or more nucleotide substitutions, insertions, or deletions. In some embodiments, the RD comprises a region of the sequence of the IND, and further comprises a region that does not have sequence identity or complementary to the IND. In some embodiments, the RD comprises a region that has substantial identity to the sequence of the IND comprising one or more nucleotide edits, and further comprises a region that does not have sequence identity or complementary to the IND. In some embodiments, the region that does not have sequence identity or complementary to the IND has a biological function or encodes a polypeptide or a portion thereof having a biological function. In some embodiments, the RD comprises one or more intended nucleotide edits compared to the IND and encodes a polypeptide or a portion thereof.

In some embodiments, the OD comprises a region (or a subset) of the sequence of the IND. In some embodiments, the OD consists of a region of the sequence of the IND. In some embodiments, the OD comprises one or more intended nucleotide edits compared to the IND. In some embodiments, the OD comprises a region(s) that has substantial sequence identity to the sequence of the IND, wherein the region(s) comprise one or more nucleotide edits compared to the sequence of the IND. For example, the OD may comprise a region that has substantial sequence identity to the sequence of the IND, wherein the region comprises one or more nucleotide substitutions, insertions, or deletions. In some embodiments, the OD comprises a region of the sequence of the IND, and further comprises a region that does not have sequence identity or complementary to the IND. In some embodiments, the OD comprises a region that has substantial identity to the sequence of the IND comprising one or more nucleotide edits, and further comprises a region that does not have sequence identity or complementarity to the IND. In some embodiments, the region that does not have sequence identity or complementarity to the IND has a biological function or encodes a polypeptide or a portion thereof having a biological function. In some embodiments, the OD comprises one or more intended nucleotide edits compared to the IND and encodes a polypeptide or a portion thereof.

In some embodiments, the first editing template comprises a region of identity to a sequence adjacent to the second nick site on the second PAM strand of the double-stranded target DNA, wherein the sequence is outside the IND. In some embodiments, the second editing template comprises a region of identity to a sequence adjacent to the first nick site on the first PAM strand of the double-stranded target DNA, wherein the sequence is outside the IND. Accordingly, in some embodiments, the first newly synthesized single-stranded DNA encoded by the first editing template comprises a region of complementarity to a sequence adjacent to the second nick site on the second PAM strand of the double-stranded target DNA, wherein the sequence is outside the IND. In some embodiments, the second newly synthesized single-stranded DNA encoded by the second editing template comprises a region of complementarity to a sequence adjacent to the first nick site on the first PAM strand of the double-stranded target DNA, wherein the sequence is outside the IND.

In some embodiments, the first newly synthesized single-stranded DNA comprises a region of complementarity to a sequence immediately adjacent to the second nick site on the second PAM strand of the double-stranded target DNA, wherein the sequence is outside the IND. In some embodiments, the second newly synthesized single-stranded DNA encoded by the second editing template comprises a region of complementarity to a sequence immediately adjacent to the first nick site on the first PAM strand of the double-stranded target DNA, wherein the sequence is outside the IND (see, e.g., FIG. 4F).

In some embodiments, the first newly synthesized single-stranded DNA comprises a region of complementarity to a sequence adjacent to the second nick site on the second PAM strand of the double-stranded target DNA, wherein the sequence is outside the IND, and is 1, 2, 3, 4, 5, 6, 7, 8, 9, or 10 nucleotides apart from the second nick site. In some embodiments, the second newly synthesized single-stranded DNA encoded by the second editing template comprises a region of complementarity to a sequence adjacent to the first nick site on the first PAM strand of the double-stranded target DNA, wherein the sequence is outside the IND, and is 1, 2, 3, 4, 5, 6, 7, 8, 9, or 10 nucleotides apart from the first nick site.

In some embodiments, the first editing template and the second editing template each comprises a region of complementarity or substantial complementarity to each other. In some embodiments, the first editing template comprises a sequence that is exogenous to the double-stranded target DNA. In some embodiments, the second editing template comprise a sequence that is exogenous to the double-stranded target DNA. In some embodiments, the sequence in the first editing template that is exogenous to the double-stranded target DNA comprises a region of complementarity or substantial complementarity to the sequence in the second editing template that is exogenous to the double-stranded target DNA. In some embodiments, the sequence in the first editing template that is exogenous to the double-stranded target DNA further comprises a region that is not complementary to the sequence in the second editing template that is exogenous to the double-stranded target DNA. In some embodiments, the sequence in the second editing template that is exogenous to the double-stranded target DNA comprises a region of complementarity or substantial complementarity to the sequence in the first editing template that is exogenous to the double-stranded target DNA. In some embodiments, the sequence in the second editing template that is exogenous to the double-stranded target DNA further comprises a region that is not complementary to the sequence in the first editing template that is exogenous to the double-stranded target DNA. In some embodiments, the first editing template comprises a sequence exogenous to the double-stranded target DNA, wherein the sequence exogenous to the double-stranded target DNA comprises a polynucleotide sequence that encodes an expression tag, for example, an affinity tag, a His tag, a V5 tag, or a FLAG tag. In some embodiments, the second editing template comprises a sequence exogenous to the double-stranded target DNA, wherein the sequence exogenous to the double-stranded target DNA comprises a polynucleotide sequence that encodes an expression tag, for example, an affinity tag, a His tag, a V5 tag, or a FLAG tag. In some embodiments, the first editing template and/or the second editing template comprises a sequence exogenous to the double-stranded target DNA, wherein the exogenous sequence comprises a recombinase recognition sequence (RSS), e.g., an attB sequence.

Accordingly, in some embodiments, the first newly synthesized single-stranded DNA and the second newly synthesized single-stranded DNA each comprises a region of complementarity or substantial complementarity to each other. In some embodiments, the first newly synthesized single-stranded DNA comprise a sequence that is exogenous to the double-stranded target DNA. In some embodiments, the second newly synthesized single-stranded DNA comprise a sequence that is exogenous to the double-stranded target DNA. In some embodiments, the sequence in the first newly synthesized single-stranded DNA that is exogenous to the double-stranded target DNA comprises a region of complementarity or substantial complementarity to the sequence in the second newly synthesized single-stranded DNA that is exogenous to the double-stranded target DNA. In some embodiments, the sequence in the first newly synthesized single-stranded DNA that is exogenous to the double-stranded target DNA further comprises a region that is not complementary to the sequence in the second newly synthesized single-stranded DNA that is exogenous to the double-stranded target DNA. In some embodiments, the sequence in the second newly synthesized single-stranded DNA that is exogenous to the double-stranded target DNA comprises a region of complementarity or substantial complementarity to the sequence in the first newly synthesized single-stranded DNA that is exogenous to the double-stranded target DNA. In some embodiments, the sequence in the second newly synthesized single-stranded DNA that is exogenous to the double-stranded target DNA further comprises a region that is not complementary to the sequence in the first newly synthesized single-stranded DNA that is exogenous to the double-stranded target DNA.

Accordingly, in some embodiments, the first newly synthesized single-stranded DNA and the second newly synthesized single-stranded DNA form an OD that comprises a sequence that is exogenous to the double-stranded target DNA, e.g., the TRAC gene. In some embodiments, the first newly synthesized single-stranded DNA and the second newly synthesized single-stranded DNA form an RD that comprises a sequence that is exogenous to the double-stranded target DNA, e.g., the TRAC gene. Through prime editing, in some embodiments, the IND is excised and is replaced by the RD. In some embodiments, the IND is excised and is replaced by the RD. Accordingly, in some embodiments, the IND in the target gene, e.g. the TRAC gene, is deleted and replaced by the exogenous sequence, e.g., one or more recombinase recognition sequences.

In some embodiments, the first editing template comprises a sequence that has complementarity or substantial complementarity to an endogenous sequence of the double-stranded target DNA, e.g., the TRAC gene. In some embodiments, the second editing template comprises a sequence that has complementarity or substantial complementarity to an endogenous sequence of the double-stranded target DNA, e.g., the TRAC gene.

In some embodiments, the sequence of the first editing template that has complementarity or substantial complementarity to an endogenous sequence of the double-stranded target DNA comprises a region of complementarity or substantial complementarity to the sequence of the second editing template that has complementarity or substantial complementarity to an endogenous sequence of the double-stranded target DNA. In some embodiments, the sequence of the first editing template that complementarity or substantial complementarity to an endogenous sequence of the double-stranded target DNA further comprises a region that is not complementary to the sequence of the second editing template that has complementarity or substantial complementarity to an endogenous sequence of the double-stranded target DNA. In some embodiments, the sequence of the second editing template that has complementarity or substantial complementarity to an endogenous sequence of the double-stranded target DNA comprises a region of complementarity or substantial complementarity to the sequence of the first editing template that has complementarity or substantial complementarity to an endogenous sequence of the double-stranded target DNA. In some embodiments, the sequence of the second editing template that has complementarity or substantial complementarity to an endogenous sequence of the double-stranded target DNA further comprises a region that is not complementary to the sequence of the first editing template that has identity or substantial identity to an endogenous sequence of the double-stranded target DNA.

Accordingly, in some embodiments, the first newly synthesized single-stranded DNA comprises a sequence that has identity or substantial identity to an endogenous sequence of the double-stranded target DNA, e.g., the TRAC gene. In some embodiments, the second newly synthesized single-stranded DNA comprises a sequence that has identity or substantial identity to an endogenous sequence of the double-stranded target DNA, e.g., the TRAC gene. In some embodiments, the first newly synthesized single-stranded DNA and/or the second newly synthesized single-stranded DNA comprises a sequence that has identity or substantial identity to an endogenous sequence of the double-stranded target DNA. In some embodiments, the first newly synthesized single-stranded DNA comprises a sequence that has identity or substantial identity to an endogenous sequence on the second strand of the double-stranded target DNA, e.g., the TRAC gene. In some embodiments, the second newly synthesized single-stranded DNA comprises a sequence that has identity or substantial identity to an endogenous sequence on the first strand of the double-stranded target DNA, e.g., the TRAC gene.

In some embodiments, the sequence of the first newly synthesized single-stranded DNA that has identity or substantial identity to an endogenous sequence of the double-stranded target DNA comprises a region of complementarity or substantial complementarity to the sequence of the second newly synthesized single-stranded DNA that has identity or substantial identity to an endogenous sequence of the double-stranded target DNA. In some embodiments, the sequence of the first newly synthesized single-stranded DNA that has identity or substantial identity to an endogenous sequence of the double-stranded target DNA further comprises a region that is not complementary to the sequence of the second newly synthesized single-stranded DNA that has identity or substantial identity to an endogenous sequence of the double-stranded target DNA. In some embodiments, the sequence of the second newly synthesized single-stranded DNA that has identity or substantial identity to an endogenous sequence of the double-stranded target DNA comprises a region of complementarity or substantial complementarity to the sequence of the first newly synthesized single-stranded DNA that has identity or substantial identity to an endogenous sequence of the double-stranded target DNA. In some embodiments, the sequence of the second newly synthesized single-stranded DNA that has identity or substantial identity to an endogenous sequence of the double-stranded target DNA further comprises a region that is not complementary to the sequence of the first newly synthesized single-stranded DNA that has identity or substantial identity to an endogenous sequence of the double-stranded target DNA.

Accordingly, in some embodiments, the first newly synthesized single-stranded DNA and the second newly synthesized single-stranded DNA form an OD that comprises an endogenous sequence of the double-stranded target DNA, e.g. the TRAC gene. In some embodiments, the first newly synthesized single-stranded DNA and the second newly synthesized single-stranded DNA form an RD that comprises an endogenous sequence of the double-stranded target DNA, e.g. the TRAC gene. In some embodiments, the RD or the OD comprises an endogenous sequence of the double-stranded target DNA, e.g., the TRAC gene. In some embodiments, the RD or the OD comprises a sequence that is exogenous compared to the double-stranded target DNA, e.g., the TRAC gene.

In some embodiments, the first editing template and/or the second editing template is partially complementary, substantially complementary, or identical to the sequence of the IND. In some embodiments, for example, the first editing template comprises a region that is complementary or identical to a region of a sequence of the IND. In some embodiments, the first editing template comprises a region of complementarity to the sequence on the first PAM strand of the IND. In some embodiments, the first editing template further comprises a region of complementarity to the second editing template. In some embodiments, the first editing template is partially complementary, substantially complementary or identical to a sequence of the IND, and is also substantially complementary to the second editing template. In some embodiments, the second editing template comprises a region that is complementary or identical to a region of a sequence of the IND. In some embodiments, the second editing template comprises a region of complementarity to the sequence on the second PAM strand of the IND. In some embodiments, the second editing template further comprises a region of complementarity to the first editing template. In some embodiments, the second editing template is partially complementary, substantially complementary or identical to a sequence of the IND, and is also substantially complementary to the first editing template. In some embodiments, the first editing template and the second editing template each comprises a region of complementarity to a sequence of the IND.

The partially complementary region(s) in the first editing template and the second editing template can be in any position within the first editing template and the second editing template. Accordingly, in some embodiments, the first newly synthesized single-stranded DNA encoded by the first editing template and the second newly synthesized single-stranded DNA encoded by the second editing template are partially complementary to each other, at any position within the first newly synthesized single-stranded DNA encoded by the first editing template and the second newly synthesized single-stranded DNA encoded by the second editing template. In some embodiments, the first newly synthesized single-stranded DNA comprises a region of complementarity to the first strand of the IND, at or near the 3′ end of the first newly synthesized single-stranded DNA. In some embodiments, the first newly synthesized single-stranded DNA comprises a region of complementarity to the first strand of the IND, at or near the 5′ end of the first newly synthesized single-stranded DNA. In some embodiments, the first newly synthesized single-stranded DNA comprises a region of complementarity to the first strand of the IND, in the middle of the first newly synthesized single-stranded DNA. In some embodiments, the second newly synthesized single-stranded DNA comprises a region of complementarity to the second strand of the IND, at or near the 3′ end of the second newly synthesized single-stranded DNA. In some embodiments, the second newly synthesized single-stranded DNA comprises a region of complementarity to the second strand of the IND, at or near the 5′ end of the second newly synthesized single-stranded DNA. In some embodiments, the second newly synthesized single-stranded DNA comprises a region of complementarity to the second strand of the IND, in the middle of the second newly synthesized single-stranded DNA. In some embodiments, the first newly synthesized single-stranded DNA and the second newly synthesized single-stranded DNA each comprises a region of complementarity to each other at the 3′ end.

Accordingly, as exemplified in FIG. 4C-FIG. 4D, in some embodiments, the first newly synthesized single-stranded DNA encoded by the first editing template comprises a region that is identical to a region of the sequence on the first PAM strand of the IND. In some embodiments, the second newly synthesized single-stranded DNA encoded by the second editing template comprises a region that is identical to a region of the sequence on the second PAM strand of the IND. In some embodiments, the first newly synthesized single-stranded DNA comprises two or more sub regions, each of which is identical to a sub region of the sequence on the first PAM strand of the IND (as exemplified in FIG. 4D). The sub regions on the first newly synthesized single-stranded DNA and/or the first PAM strand of the IND may or may not be consecutive. For example, the first newly synthesized single-stranded DNA may comprise 2 sub regions each identical to a sub region of the sequence on the first PAM strand of the IND, wherein the two sub regions of the sequence on the first PAM strand of the IND are separated by a region that does not have identity or substantial identity to the first newly synthesized single-stranded DNA. In some embodiments, the second newly synthesized single-stranded DNA comprises two or more sub regions, each of which is identical to a sub region of the sequence on the second PAM strand of the IND (as exemplified in FIG. 4D). The sub regions on the second newly synthesized single-stranded DNA and/or the second PAM strand of the IND may or may not be consecutive. For example, the second newly synthesized single-stranded DNA may comprise 2 sub regions each identical to a sub region of the sequence on the second PAM strand of the IND, wherein the two sub regions of the sequence on the second PAM strand of the IND are separated by a region that does not have identity or substantial identity to the second newly synthesized single-stranded DNA.

In some embodiments, the region of the sequence on the first PAM strand of the IND and the region of the sequence on the second PAM strand of the IND are complementary to each other. In some embodiments, the first newly synthesized single-stranded DNA encoded by the first editing template and the second newly synthesized single-stranded DNA encoded by the second editing template are at least partially complementary to each other and can anneal to each other to form an OD. In some embodiments, the first newly synthesized single-stranded DNA encoded by the first editing template and the second newly synthesized single-stranded DNA encoded by the second editing template are substantially complementary or complementary to each other and can anneal to each other to form an OD. In some embodiments, the IND is excised, and the OD is incorporated in the double-stranded target DNA at the place of the IND excision. As a result, the portion in the IND that is not complementary or identical to the first editing template or the second editing template is deleted from the double-stranded target DNA. In some embodiments, the deletion is at the 3′ end of the IND. In some embodiments, the deletion is at the 5′ end of the IND. In some embodiments, the deletion is in the middle of the IND.

In some embodiments, the first editing template of the first PEgRNA is at least partially complementary, substantially complementary, at least partially identical, or identical to a sequence of the double-stranded target DNA outside the IND. “Outside the IND” refers to sequences or positions of the double-stranded target DNA that are not in between the two nick sites generated by the first prime editor and the second prime editor. In some embodiments, the first editing template of the first PEgRNA comprises a region of identity to a sequence outside the IND on the second PAM strand (or the first strand) of the double-stranded target DNA. In some embodiments, the first editing template of the first PEgRNA comprises a region of identity to a sequence on the first strand of the double-stranded target DNA adjacent to the second nick site generated by the second prime editor complexed with the second PEgRNA, wherein the sequence is outside the IND. In some embodiments, the first editing template of the first PEgRNA comprises a region of identity to a sequence on the first strand of the double-stranded target DNA immediately adjacent to the second nick site generated by the second prime editor complexed with the second PEgRNA, wherein the sequence is outside the IND.

Accordingly, as exemplified in FIG. 4E, in some embodiments, the first newly synthesized single-stranded DNA encoded by the first editing template comprises a region of complementarity to a sequence on the first strand of the double-stranded target DNA adjacent or immediately adjacent to the second nick site generated by the second prime editor complexed with the second PERNA. In some embodiments, the first newly synthesized single-stranded DNA encoded by the first editing template comprises a region of complementarity to a sequence on the first strand of the double-stranded target DNA immediately adjacent to the second nick site generated by the second prime editor complexed with the second PERNA, wherein the sequence is outside the IND. In some embodiments, the first newly synthesized single-stranded DNA anneals with the sequence on the first strand of the double-stranded target DNA adjacent or immediately adjacent to the second nick site generated by the second prime editor. In some embodiments, through DNA repair, the IND is excised and deleted from the double-stranded target DNA, e.g., the target gene.

In some embodiments, the second editing template of the second PEgRNA is at least partially complementary, substantially complementary, at least partially identical, or identical to a sequence of the double-stranded target DNA outside the IND. In some embodiments, the second editing template of the second PERNA comprises a region of identity to a sequence outside the IND on the first PAM strand (or the second strand) of the double-stranded target DNA. In some embodiments, the second editing template of the second PEgRNA comprises a region of identity to a sequence on the second strand of the double-stranded target DNA adjacent to the first nick site generated by the first prime editor complexed with the first PERNA, wherein the sequence is outside the IND. In some embodiments, the second editing template of the second PEgRNA comprises a region of identity to a sequence on the second strand of the double-stranded target DNA immediately adjacent to the first nick site generated by the first prime editor complexed with the first PERNA, wherein the sequence is outside the IND.

Accordingly, as exemplified in FIG. 4E, in some embodiments, the second newly synthesized single-stranded DNA encoded by the second editing template comprises a region of complementarity to a sequence on the second strand of the double-stranded target DNA adjacent or immediately adjacent to the first nick site generated by the first prime editor complexed with the first PEgRNA. In some embodiments, the second newly synthesized single-stranded DNA encoded by the second editing template comprises a region of complementarity to a sequence on the second strand of the double-stranded target DNA adjacent or immediately adjacent to the first nick site generated by the first prime editor complexed with the first PEgRNA, wherein the sequence is outside the IND. In some embodiments, the second newly synthesized single-stranded DNA anneals with the sequence on the second strand of the double-stranded target DNA adjacent or immediately adjacent to the first nick site generated by the first prime editor. In some embodiments, through DNA repair, the IND is excised and deleted from the double-stranded target DNA, e.g., the target gene.

In some embodiments, the first editing template of the first PEgRNA comprises a region at least partially identical to a sequence on the first strand of the double-stranded target DNA immediately adjacent to the second nick site generated by the second prime editor complexed with the second PEgRNA, wherein the sequence is outside the IND. In some embodiments, the second editing template of the second PEgRNA comprises a region at least partially identical to a sequence on the second strand of the double-stranded target DNA immediately adjacent to the first nick site generated by the first prime editor complexed with the first PEgRNA, wherein the sequence is outside the IND. In some embodiments, the first editing template and the second editing template further comprise a region of complementarity or substantial complementarity to each other.

Accordingly, as exemplified in FIG. 4F, in some embodiments, the first newly synthesized single-stranded DNA encoded by the first editing template comprises a region of complementarity to a sequence on the first strand of the double-stranded target DNA, wherein the sequence immediately adjacent to the second nick site generated by the second prime editor complexed with the second PERNA and is outside the IND. In some embodiments, the second newly synthesized single-stranded DNA encoded by the second editing template comprises a region of complementarity or substantial complementarity to a sequence on the second strand of the double-stranded target DNA, wherein the sequence is immediately adjacent to the first nick site generated by the first prime editor complexed with the first PEgRNA and is outside the IND. In some embodiments, the first newly synthesized single-stranded DNA encoded by the first editing template and the second newly synthesized single-stranded DNA encoded by the second editing template further comprise a region of complementarity or substantial complementarity to each other, and can anneal to each other to form an OD.

In some embodiments, the first newly synthesized single-stranded DNA encoded by the first editing template further comprises a region that is not complementary to the second newly synthesized single-stranded DNA encoded by the second editing template and does not have complementarity or identity to the double-stranded target DNA. In some embodiments, the second newly synthesized single-stranded DNA encoded by the second editing template further comprises a region that is not complementary to the first newly synthesized single-stranded DNA encoded by the first editing template and does not have complementarity or identity to the double-stranded target DNA, e.g., the target gene.

Accordingly, in some embodiments, the RD comprises (i) the OD, (ii) the region of the first newly synthesized single-stranded DNA that is not complementary to the second newly synthesized single-stranded DNA and does not have complementarity or identity to the double-stranded target DNA, and a complementary sequence thereof, and (iii) the region of the second newly synthesized single-stranded DNA that is not complementary to the first newly synthesized single-stranded DNA and does not have complementarity or identity to the double-stranded target DNA, and a complementary sequence thereof. In some embodiments, through DNA repair, the IND is excised from the double-stranded target DNA, e.g., the TRAC gene, and the RD is incorporated into the double-stranded target DNA.

In some embodiments, the IND is excised and deleted from the target gene, and the RD is incorporated at the place of excision of the IND. In some embodiments, the IND is excised and deleted from the target gene, and the OD is incorporated at the place of excision of the IND. In some embodiments, the RD comprises a region of identity to an endogenous sequence of the double-stranded target DNA. In some embodiments, the OD comprises a region of identity to an endogenous sequence of the double-stranded target DNA. In some embodiments, the RD does not have sequence identity to an endogenous sequence of the double-stranded target DNA. In some embodiments, the RD is exogenous to the double-stranded target DNA, e.g., the target gene. In some embodiments, the RD has a biological function or encodes a polypeptide having a biological function. In some embodiments, the OD does not have sequence identity to an endogenous sequence of the double-stranded target DNA. In some embodiments, the OD is exogenous to the double-stranded target DNA, e.g., the target gene. In some embodiments, the OD has a biological function or encodes a polypeptide having a biological function.

In some embodiments, the first editing template of the first PEgRNA comprises a region at least partially identical to a sequence of the double-stranded target DNA that is outside the IND and is not immediately adjacent to (also referred to as “distal to”) the second nick site on the second PAM strand of the double-stranded target DNA. In some embodiments, the first editing template of the first PEgRNA comprises a region of identity to a sequence of double-stranded target DNA on the second PAM strand that is outside the IND and is at least 1, 2, 3, 4, 5, 6, 7, 8, 9, 10, 11, 12, 13, 14, 15, 16, 17, 18, 19, 20 or more nucleotides downstream of the second nick site.

In some embodiments, the second editing template of the second PEgRNA comprises a region at least partially identical to a sequence of the double-stranded target DNA that is outside the IND and is not immediately adjacent to the first nick site on the first PAM strand of the double-stranded target DNA. In some embodiments, the second editing template of the second PEgRNA comprises a region of identity to a sequence of the double-stranded target DNA on the first PAM strand that is outside the IND and is at least 1, 2, 3, 4, 5, 6, 7, 8, 9, 10, 11, 12, 13, 14, 15, 16, 17, 18, 19, 20 or more nucleotides downstream of the first nick site. In some embodiments, the second editing template of the second PEgRNA comprises a region of identity to a sequence of the double-stranded target DNA that is outside the IND and is at least 1, 2, 3, 4, 5, 6, 7, 8, 9, 10, 11, 12, 13, 14, 15, 16, 17, 18, 19, 20 or more nucleotides upstream of the first nick site.

Accordingly, in some embodiments, the first newly synthesized single-stranded DNA encoded by the first editing template comprises a region of complementarity to a sequence of the double-stranded target DNA that is outside the IND and is not immediately adjacent (i.e., distal) to the second nick site on the second PAM strand. In some embodiments, the first newly synthesized DNA encoded by the first editing template can anneal with the sequence that is outside the IND and is not immediately adjacent to the second nick site on the second PAM strand of the double-stranded target DNA. In some embodiments, the first newly synthesized DNA encoded by the first editing template comprises a region of complementarity to, and can anneal with a sequence of the double-stranded target DNA that is outside the IND and is at least 1, 2, 3, 4, 5, 6, 7, 8, 9, 10, 11, 12, 13, 14, 15, 16, 17, 18, 19, 20 or more nucleotides downstream of the second nick site.

In some embodiments, the second newly synthesized single-stranded DNA encoded by the second editing template comprises a region of complementarity to a sequence of the first PAM strand of the double-stranded target DNA that is outside the IND and is not immediately adjacent to (also referred to as “distal to”) the first nick site on the first PAM strand. In some embodiments, the second newly synthesized DNA encoded by the second editing template can anneal with the sequence that is outside the IND and is not immediately adjacent to the first nick site on the first PAM strand of the double-stranded target DNA. In some embodiments, the second newly synthesized DNA encoded by the second editing template comprises a region of complementarity to, and can anneal with a sequence of the double-stranded target DNA that is outside the IND and is at least 1, 2, 3, 4, 5, 6, 7, 8, 9, 10, 11, 12, 13, 14, 15, 16, 17, 18, 19, 20 or more nucleotides upstream of the first nick site.

In some embodiments, through DNA repair, the IND is excised and deleted from the double-stranded target DNA, e.g., the target gene. In some embodiments, the endogenous sequence of the double-stranded target DNA between the 3′ end of the sequence that is outside the IND and is distal to the second nick site on the second PAM strand and the 3′ end of the sequence of the double-stranded target DNA that is outside the IND and is distal to the first nick site on the first PAM strand of the double-stranded target DNA is excised and deleted from the double-stranded target DNA.

In some embodiments, as exemplified in FIG. 4G, the first search target sequence is downstream of the second search target sequence. In some embodiments, the first editing template comprises a region that has complementarity or substantial complementarity to the second editing template, and optionally further comprises a region that does not have a complementarity to the second editing template. In some embodiments, the second editing template comprises a region that has complementarity or substantial complementarity to the first editing template, and optionally further comprises a region that does not have complementarity to the first editing template. Accordingly, in some embodiments, the first newly synthesized single-stranded DNA comprises a region of complementarity to the second newly synthesized single-stranded DNA, and optionally further comprises a region that does not have a complementarity to the second newly synthesized single-stranded DNA, wherein the first newly synthesized single-stranded DNA is downstream of the second newly synthesized single-stranded DNA. In some embodiments, the second newly synthesized single-stranded DNA comprises a region of complementarity to the first newly synthesized single-stranded DNA, and optionally further comprises a region that does not have a complementarity to the first newly synthesized single-stranded DNA, wherein the first newly synthesized single-stranded DNA is downstream of the second newly synthesized single-stranded DNA. Through DNA repair, the sequence of the OD or the RD is incorporated in the double-stranded target DNA, and the IND sequence is duplicated in the double-stranded target DNA.

### Prime Editor

The term “prime editor (PE)” refers to the polypeptide or polypeptide components involved in prime editing. In various embodiments, a prime editor includes a polypeptide domain having DNA binding activity and a polypeptide domain having DNA polymerase activity. In some embodiments, the polypeptide domain having DNA binding activity is a polypeptide domain having programmable DNA binding activity. In some embodiments, the prime editor further comprises a polypeptide domain having nuclease activity. In some embodiments, the polypeptide domain having DNA binding activity comprises a nuclease domain or nuclease activity. In some embodiments, the polypeptide domain having nuclease activity comprises a nickase, or a fully active nuclease. As used herein, the term “nickase” refers to a nuclease capable of cleaving only one strand of a double-stranded DNA target. In some embodiments, the prime editor comprises a polypeptide domain that is an inactive nuclease. In some embodiments, the polypeptide domain having programmable DNA binding activity comprises a nucleic acid guided DNA binding domain, for example, a CRISPR-Cas protein, for example, a Cas9 nickase, a Cpf1 nickase, or another CRISPR-Cas nuclease. In some embodiments, the polypeptide domain having DNA polymerase activity comprises a template-dependent DNA polymerase, for example, a DNA-dependent DNA polymerase or an RNA-dependent DNA polymerase. In some embodiments, the DNA polymerase is a reverse transcriptase. In some embodiments, the prime editor comprises additional polypeptides or polypeptide domains involved in prime editing, for example, a polypeptide domain having 5′ endonuclease activity, e.g., a 5′ endogenous DNA flap endonucleases (e.g., FEN1), for helping to drive the prime editing process towards the edited product formation. In some embodiments, the prime editor further comprises an RNA-protein recruitment polypeptide, for example, a MS2 coat protein.

A prime editor may be engineered. In some embodiments, the polypeptide components of a prime editor do not naturally occur in the same organism or cellular environment. In some embodiments, the polypeptide components of a prime editor may be of different origins or from different organisms. In some embodiments, a prime editor comprises a DNA binding domain and a DNA polymerase domain that are derived from different species. In some embodiments, a prime editor comprises a Cas polypeptide (DNA binding domain) and a reverse transcriptase polypeptide (DNA polymerase) that are derived from different species. For example, a prime editor may comprise a S. pyogenes Cas9 polypeptide and a Moloney murine leukemia virus (M-MLV) reverse transcriptase polypeptide.

In some embodiments, polypeptide domains of a prime editor may be fused or linked by a peptide linker to form a fusion protein. In other embodiments, a prime editor comprises one or more polypeptide domains provided in trans as separate proteins, which are capable of being associated to each other through non-peptide linkages or through aptamers or recruitment sequences. For example, a prime editor may comprise a DNA binding domain and a reverse transcriptase domain associated with each other by an RNA-protein recruitment aptamer, e.g., a MS2 aptamer, which may be linked to a PEgRNA. Prime editor polypeptide components may be encoded by one or more polynucleotides in whole or in part. In some embodiments, a single polynucleotide, construct, or vector encodes the prime editor fusion protein. In some embodiments, multiple polynucleotides, constructs, or vectors each encode a polypeptide domain or portion of a domain of a prime editor, or a portion of a prime editor fusion protein. For example, a prime editor fusion protein may comprise an N-terminal portion fused to an intein-N and a C-terminal portion fused to an intein-C, each of which is individually encoded by an AAV vector.

The term “prime editor complex” is used interchangeably with the term “prime editing complex” and refers to a complex comprising one or more prime editor components (e.g., a polypeptide domain having DNA binding activity and a polypeptide domain having DNA polymerase activity) complexed with a PEgRNA.

### Prime Editor Nucleotide Polymerase Domain

In some embodiments, a prime editor comprises a nucleotide polymerase domain, e.g., a DNA polymerase domain. The DNA polymerase domain may be a wild-type DNA polymerase domain, a full-length DNA polymerase protein domain, or may be a functional mutant, a functional variant, or a functional fragment thereof. In some embodiments, the polymerase domain is a template dependent polymerase domain. For example, the DNA polymerase may rely on a template polynucleotide strand, e.g., the editing template sequence, for new strand DNA synthesis. In some embodiments, the prime editor comprises a DNA-dependent DNA polymerase. For example, a prime editor having a DNA-dependent DNA polymerase can synthesize a new single-stranded DNA using a PEgRNA editing template that comprises a DNA sequence as a template. In such cases, the PEgRNA is a chimeric or hybrid PEgRNA, and comprises an extension arm comprising a DNA strand. As used herein, an “extension arm” is a polynucleotide portion of a PERNA that comprises an editing template and a primer binding site sequence (PBS). In some embodiments, an extension arm further comprises additional components, for example, a 3′ modifier. The chimeric or hybrid PEgRNA may comprise an RNA portion (including the spacer and the gRNA core) and a DNA portion (the extension arm comprising the editing template that includes a strand of DNA).

The DNA polymerases can be wild-type polymerases from eukaryotic, prokaryotic, archaeal, or viral organisms, and/or the polymerases may be modified by genetic engineering, mutagenesis, or directed evolution-based processes. The polymerases can be a T7 DNA polymerase, T5 DNA polymerase, T4 DNA polymerase, Klenow fragment DNA polymerase, DNA polymerase III and the like. The polymerases can be thermostable, and can include Taq, Tne, Tma, Pfu, Tfl, Tth, Stoffel fragment, VENT® and DEEPVENT® DNA polymerases, KOD, Tgo, JDF3, and mutants, variants and derivatives thereof.

In some embodiments, the DNA polymerase is a bacteriophage polymerase, for example, a T4, T7, or phi29 DNA polymerase. In some embodiments, the DNA polymerase is an archaeal polymerase, for example, pol I type archaeal polymerase or a pol II type archaeal polymerase. In some embodiments, the DNA polymerase comprises a thermostable archaeal DNA polymerase. In some embodiments, the DNA polymerase comprises a eubacterial DNA polymerase, for example, Pol I, Pol II, or Pol III polymerase. In some embodiments, the DNA polymerase is a Pol I family DNA polymerase. In some embodiments, the DNA polymerase is an E. coli Pol I DNA polymerase. In some embodiments, the DNA polymerase is a Pol II family DNA polymerase. In some embodiments, the DNA polymerase is a Pyrococcus furiosus (Pfu) Pol II DNA polymerase. In some embodiments, the DNA polymerase is a Pol IV family DNA polymerase. In some embodiments, the DNA polymerase is an E. coli Pol IV DNA polymerase.

In some embodiments, the DNA polymerase comprises a eukaryotic DNA polymerase. In some embodiments, the DNA polymerase is a Pol-beta DNA polymerase, a Pol-lambda DNA polymerase, a Pol-sigma DNA polymerase, or a Pol-mu DNA polymerase. In some embodiments, the DNA polymerase is a Pol-alpha DNA polymerase. In some embodiments, the DNA polymerase is a POLA1 DNA polymerase. In some embodiments, the DNA polymerase is a POLA2 DNA polymerase. In some embodiments, the DNA polymerase is a Pol-delta DNA polymerase. In some embodiments, the DNA polymerase is a POLD1 DNA polymerase. In some embodiments, the DNA polymerase is a POLD2 DNA polymerase. In some embodiments, the DNA polymerase is a human POLD1 DNA polymerase. In some embodiments, the DNA polymerase is a human POLD2 DNA polymerase. In some embodiments, the DNA polymerase is a POLD3 DNA polymerase. In some embodiments, the DNA polymerase is a POLD4 DNA polymerase. In some embodiments, the DNA polymerase is a Pol-epsilon DNA polymerase. In some embodiments, the DNA polymerase is a POLE1 DNA polymerase. In some embodiments, the DNA polymerase is a POLE2 DNA polymerase. In some embodiments, the DNA polymerase is a POLE3 DNA polymerase. In some embodiments, the DNA polymerase is a Pol-eta (POLH) DNA polymerase. In some embodiments, the DNA polymerase is a Pol-iota (POLI) DNA polymerase. In some embodiments, the DNA polymerase is a Pol-kappa (POLK) DNA polymerase. In some embodiments, the DNA polymerase is a Rev1 DNA polymerase. In some embodiments, the DNA polymerase is a human Rev1 DNA polymerase. In some embodiments, the DNA polymerase is a viral DNA-dependent DNA polymerase. In some embodiments, the DNA polymerase is a B family DNA polymerases. In some embodiments, the DNA polymerase is a herpes simplex virus (HSV) UL30 DNA polymerase. In some embodiments, the DNA polymerase is a cytomegalovirus (CMV) UL54 DNA polymerase.

In some embodiments, the DNA polymerase is an archaeal polymerase. In some embodiments, the DNA polymerase is a Family B/pol I type DNA polymerase. For example, in some embodiments, the DNA polymerase is a homolog of Pfu from Pyrococcus furiosus. In some embodiments, the DNA polymerase is a pol II type DNA polymerase. For example, in some embodiments, the DNA polymerase is a homolog of P. furiosus DP1/DP2 2-subunit polymerase. In some embodiments, the DNA polymerase lacks 5′ to 3′ nuclease activity. Suitable DNA polymerases (pol I or pol II) can be derived from archaea with optimal growth temperatures that are similar to the desired assay temperatures.

In some embodiments, the DNA polymerase comprises a thermostable archaeal DNA polymerase. In some embodiments, the thermostable DNA polymerase is isolated or derived from Pyrococcus species (furiosus, species GB-D, woesii, abysii, horikoshii), Thermococcus species (kodakaraensis KOD1, litoralis, species 9 degrees North-7, species JDF-3, gorgonarius), Pyrodictium occultum, and Archaeoglobus fulgidus.

Polymerases may also be from eubacterial species. In some embodiments, the DNA polymerase is a Pol I family DNA polymerase. In some embodiments, the DNA polymerase is an E. coli Pol I DNA polymerase. In some embodiments, the DNA polymerase is a Pol II family DNA polymerase. In some embodiments, the DNA polymerase is a Pyrococcus furiosus (Pfu) Pol II DNA polymerase. In some embodiments, the DNA polymerase is a Pol III family DNA polymerase. In some embodiments, the DNA polymerase is a Pol IV family DNA polymerase. In some embodiments, the DNA polymerase is an E. coli Pol IV DNA polymerase. In some embodiments, the Pol I DNA polymerase is a DNA polymerase functional variant that lacks or has reduced 5′ to 3′ exonuclease activity.

Suitable thermostable pol I DNA polymerases can be isolated from a variety of thermophilic eubacteria, including Thermus species and Thermotoga maritima such as Thermus aquaticus (Taq), Thermus thermophilus (Tth) and Thermotoga maritima (Tma UITma).

In some embodiments, a prime editor comprises an RNA-dependent DNA polymerase domain, for example, a reverse transcriptase (RT). A RT or an RT domain may be a wild-type RT domain, a full-length RT domain, or may be a functional mutant, a functional variant, or a functional fragment thereof. An RT or an RT domain of a prime editor may comprise a wild-type RT, or may be engineered or evolved to contain specific amino acid substitutions, truncations, or variants. An engineered RT may comprise sequences or amino acid changes different from a naturally occurring RT. In some embodiments, the engineered RT may have improved reverse transcription activity over a naturally occurring RT or RT domain. In some embodiments, the engineered RT may have improved features over a naturally occurring RT, for example, improved thermostability, reverse transcription efficiency, or target fidelity. In some embodiments, a prime editor comprising the engineered RT has improved prime editing efficiency over a prime editor having a reference naturally occurring RT.

In some embodiments, a prime editor comprises a virus RT, for example, a retrovirus RT. Non-limiting examples of virus RT include Moloney murine leukemia virus (M-MLV, MMLVRT, M-MLV RT, or MLVRT); human T-cell leukemia virus type 1 (HTLV-1) RT; bovine leukemia virus (BLV) RT; Rous Sarcoma Virus (RSV) RT; human immunodeficiency virus (HIV) RT, M-MFV RT, Avian Sarcoma-Leukosis Virus (ASLV) RT, Rous Sarcoma Virus (RSV) RT, Avian Myeloblastosis Virus (AMV) RT, Avian Erythroblastosis Virus (AEV) Helper Virus MCAV RT, Avian Myelocytomatosis Virus MC29 Helper Virus MCAV RT, Avian Reticuloendotheliosis Virus (REV-T) Helper Virus REV-A RT, Avian Sarcoma Virus UR2 Helper Virus (UR2AV) RT, Avian Sarcoma Virus Y73 Helper Virus YAV RT, Rous Associated Virus (RAV) RT, and Myeloblastosis Associated Virus (MAV) RT, all of which may be suitably used in the methods and composition described herein.

In some embodiments, the prime editor comprises a wild type M-MLV RT, a functional mutant, a functional variant, or a functional fragment thereof. Table 1A provides sequences of illustrative M-MLV RTs suitable for use with compositions and methods of the disclosure.

In some embodiments, a prime editor comprises a wild-type M-MLV RT as set forth in SEQ ID NO: 1002. In some embodiments, a prime editor comprises a variant M-MLV RT as set forth in SEQ ID NO: 1001 In some embodiments, a prime editor comprises a variant M-MLV RT as set forth in SEQ ID NO: 1003.

TABLE 1A
Illustrative M-MLV RT Sequences

| SEQ | | |
|---|---|---|
| ID | Sequence | |
| NO: | description | Amino acid sequence |
| 1002 | Wild-type | TLNIEDEHRLHETSKEPDVSLGSTWLSDFPQAWAETGGMGLAVRQA |
| | M-MLV RT | PLIIPLKATSTPVSIKQYPMSQEARLGIKPHIQRLLDQGILVPCQSPWNT |
| | | PLLPVKKPGTNDYRPVQDLREVNKRVEDIHPTVPNPYNLLSGLPPSH |
| | | QWYTVLDLKDAFFCLRLHPTSQPLFAFEWRDPEMGISGQLTWTRLPQ |
| | | GFKNSPTLFDEALHRDLADFRIQHPDLILLQYVDDLLLAATSELDCQQ |
| | | GTRALLQTLGNLGYRASAKKAQICQKQVKYLGYLLKEGQRWLTEA |
| | | RKETVMGQPTPKTPRQLREFLGTAGFCRLWIPGFAEMAAPLYPLTKT |
| | | GTLFNWGPDQQKAYQEIKQALLTAPALGLPDLTKPFELFVDEKQGY |
| | | AKGVLTQKLGPWRRPVAYLSKKLDPVAAGWPPCLRMVAAIAVLTK |
| | | DAGKLTMGQPLVILAPHAVEALVKQPPDRWLSNARMTHYQALLLD |
| | | TDRVQFGPVVALNPATLLPLPEEGLQHNCLDILAEAHGTRPDLTDQP |
| | | LPDADHTWYTDGSSLLQEGQRKAGAAVTTETEVIWAKALPAGTSAQ |
| | | RAELIALTQALKMAEGKKLNVYTDSRYAFATAHIHGEIYRRRGLLTS |
| | | EGKEIKNKDEILALLKALFLPKRLSIIHCPGHQKGHSAEARGNRMAD |
| | | QAARKAAITETPDTSTLLIENSSP |
| 1001 | Variant | TLNIEDEYRLHETSKEPDVSLGSTWLSDFPQAWAETGGMGLAVRQA |
| | M-MLV RT | PLIIPLKATSTPVSIKQYPMSQEARLGIKPHIQRLLDQGILVPCQSPWNT |
| | | PLLPVKKPGTNDYRPVQDLREVNKRVEDIHPTVPNPYNLLSGLPPSH |
| | | QWYTVLDLKDAFFCLRLHPTSQPLFAFEWRDPEMGISGQLTWTRLPQ |
| | | GFKNSPTLFDEALHRDLADFRIQHPDLILLQYVDDLLLAATSELDCQQ |
| | | GTRALLQTLGNLGYRASAKKAQICQKQVKYLGYLLKEGQRWLTEA |
| | | RKETVMGQPTPKTPRQLREFLGTAGFCRLWIPGFAEMAAPLYPLTKT |
| | | GTLFNWGPDQQKAYQEIKQALLTAPALGLPDLTKPFELFVDEKQGY |
| | | AKGVLTQKLGPWRRPVAYLSKKLDPVAAGWPPCLRMVAAIAVLTK |
| | | DAGKLTMGQPLVILAPHAVEALVKQPPDRWLSNARMTHYQALLLD |
| | | TDRVQFGPVVALNPATLLPLPEEGLQHNCLDILAEAHGTRPDLTDQP |
| | | LPDADHTWYTDGSSLLQEGQRKAGAAVTTETEVIWAKALPAGTSAQ |
| | | RAELIALTQALKMAEGKKLNVYTDSRYAFATAHIHGEIYRRRGLLTS |
| | | EGKEIKNKDEILALLKALFLPKRLSIIHCPGHQKGHSAEARGNRMAD |
| | | QAARKAAITETPDTSTLLIENSSP |
| 1003 | Variant | TLNIEDEYRLHETSKEPDVSLGSTWLSDFPQAWAETGGMGLAVRQA |
| | M-MLV RT | PLIIPLKATSTPVSIKQYPMSQEARLGIKPHIQRLLDQGILVPCQSPWNT |
| | (D200N, | PLLPVKKPGTNDYRPVQDLREVNKRVEDIHPTVPNPYNLLSGLPPSH |
| | T330P, | QWYTVLDLKDAFFCLRLHPTSQPLFAFEWRDPEMGISGQLTWTRLPQ |
| | L603W, | GFKNSPTLFNEALHRDLADFRIQHPDLILLQYVDDLLLAATSELDCQQ |
| | T306K, | GTRALLQTLGNLGYRASAKKAQICQKQVKYLGYLLKEGQRWLTEA |
| | W313F | RKETVMGQPTPKTPRQLREFLGKAGFCRLFIPGFAEMAAPLYPLTKP |
| | Pentamutant) | GTLFNWGPDQQKAYQEIKQALLTAPALGLPDLTKPFELFVDEKQGY |
| | | AKGVLTQKLGPWRRPVAYLSKKLDPVAAGWPPCLRMVAAIAVLTK |
| | | DAGKLTMGQPLVILAPHAVEALVKQPPDRWLSNARMTHYQALLLD |
| | | TDRVQFGPVVALNPATLLPLPEEGLQHNCLDILAEAHGTRPDLTDQP |
| | | LPDADHTWYTDGSSLLQEGQRKAGAAVTTETEVIWAKALPAGTSAQ |
| | | RAELIALTQALKMAEGKKLNVYTDSRYAFATAHIHGEIYRRRGWLTS |
| | | EGKEIKNKDEILALLKALFLPKRLSIIHCPGHQKGHSAEARGNRMAD |
| | | QAARKAAITETPDTSTLLIENSSP |
| 1004 | Variant | TLNIEDEYRLHETSKEPDVSLGSTWLSDFPQAWAETGGMGLAVRQA |
| | M-MLV RT | PLIIPLKATSTPVSIKQYPMSQEARLGIKPHIQRLLDQGILVPCQSPWNT |
| | (D200N, | PLLPVKKPGTNDYRPVQDLREVNKRVEDIHPTVPNPYNLLSGLPPSH |
| | T306K, | QWYTVLDLKDAFFCLRLHPTSQPLFAFEWRDPEMGISGQLTWTRLPQ |
| | W313F, | GFKNSPTLFNEALHRDLADFRIQHPDLILLQYVDDLLLAATSELDCQQ |
| | T330P, and | GTRALLQTLGNLGYRASAKKAQICQKQVKYLGYLLKEGQRWLTEA |
| | C-terminal | RKETVMGQPTPKTPRQLREFLGKAGFCRLFIPGFAEMAAPLYPLTKP |
| | truncation | GTLFNWGPDQQKAYQEIKQALLTAPALGLPDLTKPFELFVDEKQGY |
| | between | AKGVLTQKLGPWRRPVAYLSKKLDPVAAGWPPCLRMVAAIAVLTK |
| | D497/I498) | DAGKLTMGQPLVILAPHAVEALVKQPPDRWLSNARMTHYQALLLD |
| | | TDRVQFGPVVALNPATLLPLPEEGLQHNCLDNSRLIN |

In some embodiments, the prime editor comprises a M-MLV RT comprising one or more of amino acid substitutions P51X, S67X, E69X, L139X, T197X, D200X, H204X, F209X, E302X, T306X, F309X, W313X, T330X, L345X, L435X, N454X, D524X, E562X, D583X, H594X, L603X, E607X, or D653X as compared to a reference M-MLV RT, where X is any amino acid other than the original amino acid in the reference M-MLV RT. In some embodiments, the prime editor comprises a M-MLV RT comprising one or more of amino acid substitutions P51L, S67K, E69K, L139P, T197A, D200N, H204R, F209N, E302K, E302R, T306K, F309N, W313F, T330P, L345G, L435G, N454K, D524G, E562Q, D583N, H594Q, L603W, E607K, or D653N as compared to a reference M-MLV RT. In some embodiments, the reference M-MLV RT is a variant M-MLV RT as set forth in SEQ ID NO: 1001. In some embodiments, the M-MLV RT is a WT M-MLV RT as set forth in SEQ ID NO: 1002. In some embodiments, the prime editor comprises a M-MLV RT comprising one or more amino acid substitutions D200N, T330P, L603W, T306K, or W313F as compared to a reference M-MLV RT. In some embodiments, a prime editor comprises a M-MLV RT comprising amino acid substitutions D200N, T330P, L603W, T306K, and W313F as compared to a reference M-MLV RT. In some embodiments, the reference M-MLV RT is a variant M-MLV RT as set forth in SEQ ID NO: 1001. In some embodiments, the reference M-MLV RT is a WT M-MLV RT as set forth in SEQ ID NO: 1002.

In some embodiments, an RT variant may be a functional fragment of a reference RT that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or up to 100, or up to 200, or up to 300, or up to 400, or up to 500 or more amino acid changes compared to a reference RT,. In some embodiments, the RT variant comprises a fragment of a reference RT, such that the fragment is about 70% identical, about 80% identical, about 90% identical, about 95% identical, about 96% identical, about 97% identical, about 98% identical, about 99% identical, about 99.5% identical, or about 99.9% identical to the corresponding fragment of the reference RT. In some embodiments, the fragment is 30%, 35%, 40%, 45%, 50%, 55%, 60%, 65%, 70%, 75%, 80%, 85%, 90%, 95%, 96%, 97%, 98%, 99%, or 99.5% identical of the amino acid length of a corresponding reference RT (M-MLV reverse transcriptase). A reference RT can be any one of the RTs shown in Table 1A.

In some embodiments, the RT functional fragment is at least 100 amino acids in length. In some embodiments, the fragment is at least 100, 150, 200, 250, 300, 350, 400, 450, 500, 550, or up to 600 or more amino acids in length.

In still other embodiments, the functional RT variant is truncated at the N-terminus or the C-terminus, or both, by a certain number of amino acids which results in a truncated variant which still retains sufficient DNA polymerase function. In some embodiments, the functional RT variant, e.g., a functional MMLV RT variant, is truncated at the C-terminus to abolish or reduce RNAse H activity and still retain DNA polymerase activity. In some embodiments, the function RT variant has a truncation of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, 40, 50, 60, 70, 80, 90, 100, 110, 120, 130, 140, 150, 160, 170, 180, 190, 200, 210, 220, 230, 240, or 250 amino acids at the N-terminal end compared to a reference RT, e.g., a wild-type RT. In other embodiments, the RT truncated variant has a truncation of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, 40, 50, 60, 70, 80, 90, 100, 110, 120, 130, 140, 150, 160, 170, 180, 190, 200, 210, 220, 230, 240, or 250 amino acids at the C-terminal end compared to a reference RT, e.g., a wild-type RT. In some embodiments, the reference RT is a wild-type M-MLV RT. In still other embodiments, the RT truncated variant has a truncation at the N-terminal and the C-terminal end compared to a reference RT, e.g., a wild-type RT. In some embodiments, the N-terminal truncation and the C-terminal truncation are of the same length. In some embodiments, the N-terminal truncation and the C-terminal truncation are of different lengths.

For example, the prime editors disclosed herein may include a functional variant of a wild-type M-MLV reverse transcriptase. In some embodiments, the prime editor comprises a functional variant of a wild-type M-MLV RT, wherein the functional variant of M-MLV RT is truncated after amino acid position 502 compared to a reference M-MLV RT. In some embodiments, the functional variant of M-MLV RT further comprises a D200X, T306X, W313X, and/or T330X amino acid substitution compared to a reference M-MLV RT, wherein X is any amino acid other than the original amino acid in the reference M-MLV RT. In some embodiments, the functional variant of M-MLV RT further comprises a D200N, T306K, W313F, and/or T330P amino acid substitution compared to a reference M-MLV RT, wherein X is any amino acid other than the original amino acid in the reference M-MLV RT. In some embodiments, the reference M-MLV RT is a variant M-MLV RT as set forth in SEQ ID NO: 1001. In some embodiments, the M-MLV RT is a WT M-MLV RT as set forth in SEQ ID NO: 1002. A DNA sequence encoding a prime editor comprising this truncated RT is 522 bp smaller than a prime editor comprising a full-length M-MLV RT, and therefore makes its potentially useful for applications where delivery of the DNA sequence is challenging due to its size (e.g., adeno-associated virus and lentivirus delivery). In some embodiments, a prime editor comprises a M-MLV RT that comprises an amino acid sequence that is at least 70%, at least 75%, at least 80%, at least 85%, at least 90%, at least 95%, at least 96%, at least 97%, at least 98%, at least 99%, at least 99.5%, or at least 99.9% identical to an amino acid sequence set forth in in Table 1A. In some embodiments, the prime editor comprises a M-MLV RT that comprises an amino acid sequence that is selected from the group consisting of: amino acid sequences provided in Table 1A or a variant or fragment thereof. In some embodiments, the prime editor comprises a variant M-MLV RT that comprises an amino acid sequence set forth in SEQ ID NO: 1003. In some embodiments, the prime editor comprises a variant M-MLV RT that comprises an amino acid sequence set forth in SEQ ID NO: 1004.

In some embodiments, a prime editing composition or a prime editing system disclosed herein comprises a polynucleotide (e.g., a DNA, a RNA, e.g., a mRNA) that encodes a M-MLV RT. In some embodiments, the polynucleotide encodes a M-MLV RT that comprises an amino acid sequence that is at least 70%, at least 75%, at least 80%, at least 85%, at least 90%, at least 95%, at least 96%, at least 97%, at least 98%, at least 99%, at least 99.5%, or at least 99.9% identical to an amino acid sequence set forth in Table 1A. In some embodiments, the polynucleotide encodes a M-MLV RT that comprises an amino acid sequence that is at least 70%, at least 75%, at least 80%, at least 85%, at least 90%, at least 95%, at least 96%, at least 97%, at least 98%, at least 99%, at least 99.5%, or at least 99.9% identical to an amino acid sequence set forth in SEQ ID NO: 1001, 1002, 1003 or 1004. In some embodiments, the polynucleotide encodes a M-MLV RT that comprises an amino acid sequence that is selected from the group consisting of: the amino acid sequences provided in Table 1A. In some embodiments, the polynucleotide encodes a variant M-MLV RT that comprises an amino acid sequence that is set forth in SEQ ID NO: 1003. In some embodiments, the polynucleotide encodes a variant M-MLV RT that comprises an amino acid sequence that is set forth in SEQ ID NO: 1004.

In some embodiments, a prime editor comprises a eukaryotic RT, for example, a yeast, drosophila, rodent, or primate RT. In some embodiments, the prime editor comprises a Group II intron RT, for example, a. Geobacillus stearothermophilus Group II Intron (GsI-IIC) RT or a Eubacterium rectale group II intron (Eu.re.12) RT. In some embodiments, the prime editor comprises a retron RT.

### Programmable DNA Binding Domain

In some embodiments, the DNA-binding domain of a prime editor is a programmable DNA binding domain. A programmable DNA binding domain refers to a protein domain that is designed to bind a specific nucleic acid sequence, e.g., a target DNA or a target RNA. In some embodiments, the DNA-binding domain is a polynucleotide programmable DNA-binding domain that can associate with a guide polynucleotide (e.g., a PEgRNA) that guides the DNA-binding domain to a specific DNA sequence, e.g., a search target sequence in a target gene. In some embodiments, the DNA-binding domain comprises a Clustered Regularly Interspaced Short Palindromic Repeats (CRISPR) Associated (Cas) protein. A Cas protein may comprise any Cas protein described herein or a functional fragment or functional variant thereof. In some embodiments, a DNA-binding domain may also comprise a zinc-finger protein domain. In other cases, a DNA-binding domain comprises a transcription activator-like effector domain (TALE). In some embodiments, the DNA-binding domain comprises a DNA nuclease. For example, the DNA-binding domain of a prime editor may comprise an RNA-guided DNA endonuclease, e.g., a Cas protein. In some embodiments, the DNA-binding domain comprises a zinc finger nuclease (ZFN) or a transcription activator like effector domain nuclease (TALEN), where one or more zinc finger motifs or TALE motifs are associated with one or more nucleases, e.g., a Fok I nuclease domain.

In some embodiments, the DNA-binding domain comprises a nuclease activity. In some embodiments, the DNA-binding domain of a prime editor comprises an endonuclease domain having single-strand DNA cleavage activity. For example, the endonuclease domain may comprise a FokI nuclease domain. In some embodiments, the DNA-binding domain of a prime editor comprises a nuclease having full nuclease activity. In some embodiments, the DNA-binding domain of a prime editor comprises a nuclease having modified or reduced nuclease activity as compared to a wild-type endonuclease domain. For example, the endonuclease domain may comprise one or more amino acid substitutions as compared to a wild-type endonuclease domain. In some embodiments, the DNA-binding domain of a prime editor has a nickase activity. In some embodiments, the DNA-binding domain of a prime editor comprises a Cas protein domain that is a nickase. In some embodiments, compared to a wild-type Cas protein, the Cas nickase comprises one or more amino acid substitutions in a nuclease domain that reduces or abolishes its double-strand nuclease activity but retains DNA binding activity. In some embodiments, the Cas nickase comprises an amino acid substitution in a HNH domain. In some embodiments, the Cas nickase comprises an amino acid substitution in a RuvC domain.

In some embodiments, the DNA-binding domain comprises a CRISPR associated protein (Cas protein) domain. A Cas protein may be a Class 1 or a Class 2 Cas protein. A Cas protein can be a type I, type II, type III, type IV, type V Cas protein, or a type VI Cas protein. Non-limiting examples of Cas proteins include Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9 (e.g., Csn1 or Csx12), Cas10, CaslOd, Cas12a/Cpf1, Cas12b/C2c1, Cas12c/C2c3, Cas12d/CasY, Cas12e/CasX, Cas12g, Cas12h, Cas12i, Csy1, Csy2, Csy3, Csy4, Cse1, Cse2, Cse3, Cse4, Cse5e, Csc1, Csc2, Csa5, Csnl, Csn2, Csml, Csm2, Csm3, Csm4, Csm5, Csm6, Cmr1, Cmr3, Cmr4, Cmr5, Cmr6, Csbl, Csb2, Csb3, Csx17, Csx14, Csx10, Csx16, CsaX, Csx3, Csxl, Csx1S, Csx11, Csf1, Csf2, CsO, Csf4, Csdl, Csd2, Cstl, Cst2, Cshl, Csh2, Csal, Csa2, Csa3, Csa4, Csa5, Type II Cas effector proteins, Type V Cas effector proteins, Type VI Cas effector proteins, CARF, DinG, Cpf1, Cas12b/C2c1, Cas12c/C2c3, Cas12b/C2c1, Cas12c/C2c3, SpCas9 (K855A), eSpCas9 (1.1), SpCas9-HF1, hyper accurate Cas9 variant (HypaCas9), Cas Φ, Cas12b2, Cas12c (C2c3), C2c4, C2c8, C2c5, C2c10, C2c9, Cas14a, Cas14b, Cas14c, Cas14d, Cas14e, Cas14f, Cas14g, Cas14h, Cas14u, Cns2, Cas @, and homologues, modified or engineered variants, mutants, and/or functional fragments thereof. A Cas protein can be a chimeric Cas protein that is fused to other proteins or polypeptides. A Cas protein can be a chimera of various Cas proteins, for example, comprising domains of Cas proteins from different organisms.

A Cas protein, e.g., Cas9, can be from any suitable organism. In some aspects, the organism is Streptococcus pyogenes (S. pyogenes). In some aspects, the organism is Staphylococcus aureus (S. aureus). In some aspects, the organism is Streptococcus thermophilus (S. thermophilus). In some embodiments, the organism is Staphylococcus lugdunensis.

Non-limiting examples of suitable organism include Streptococcus pyogenes, Streptococcus thermophilus, Streptococcus sp., Staphylococcus aureus, Nocardiopsis dassonvillei, Streptomyces pristinae spiralis, Streptomyces viridochromo genes, Streptomyces viridochromogenes, Streptosporangium roseum, Streptosporangium roseum, AlicyclobacHlus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Pseudomonas aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Leptotrichia shahii, and Francisella novicida. In some embodiments, the organism is Streptococcus pyogenes (S. pyogenes). In some embodiments, the organism is Staphylococcus aureus (S. aureus). In some embodiments, the organism is Streptococcus thermophilus (S. thermophilus). In some embodiments, the organism is Staphylococcus lugdunensis (S. lugdunensis).

In some embodiments, a Cas protein can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractor salsuginis, Sphaerochaeta globus, Fibrobacter succinogenes subsp. Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinella succinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida.

in some embodiments, a Cas protein, e.g., Cas9, can be a wild-type or a modified form of a Cas protein. In some embodiments, a Cas protein, e.g., Cas9, can be a nuclease active variant, nuclease inactive variant, a nickase, or a functional variant or functional fragment of a wild-type Cas protein. In some embodiments, a Cas protein, e.g., Cas9, can comprise an amino acid change such as a deletion, insertion, substitution, fusion, chimera, or any combination thereof relative to a corresponding wild-type version of the Cas protein. In some embodiments, a Cas protein can be a polypeptide with at least about 50%, 60%, 70%, 80%, 90%, 91%, 92%, 93%, 94%, 95%, 96%, 97%, 98%, 99%, or 100% sequence identity or sequence similarity to a wild-type exemplary Cas protein.

A Cas protein, e.g., Cas9, may comprise one or more domains. Non-limiting examples of Cas domains include, guide nucleic acid recognition and/or binding domain, nuclease domains (e.g., DNase or RNase domains, RuvC, HNH), DNA binding domain, RNA binding domain, helicase domains, protein-protein interaction domains, and dimerization domains. In various embodiments, a Cas protein comprises a guide nucleic acid recognition and/or binding domain that can interact with a guide nucleic acid, and one or more nuclease domains that comprise catalytic activity for nucleic acid cleavage.

In some embodiments, a Cas protein, e.g., Cas9, comprises one or more nuclease domains. A Cas protein can comprise an amino acid sequence having at least about 50%, 60%, 70%, 80%, 90%, 91%, 92%, 93%, 94%, 95%, 96%, 97%, 98%, 99%, or 100% sequence identity to a nuclease domain (e.g., RuvC domain, HNH domain) of a wild-type Cas protein. In some embodiments, a Cas protein comprises a single nuclease domain. For example, a Cpf1 may comprise a RuvC domain but lacks HNH domain. In some embodiments, a Cas protein comprises two nuclease domains, e.g., a Cas9 protein can comprise an HNH nuclease domain and a RuvC nuclease domain.

In some embodiments, a prime editor comprises a Cas protein, e.g., Cas9, wherein all nuclease domains of the Cas protein are active. In some embodiments, a prime editor comprises a Cas protein having one or more inactive nuclease domains. One or a plurality of the nuclease domains (e.g., RuvC, HNH) of a Cas protein can be deleted or mutated so that they are no longer functional or comprise reduced nuclease activity. In some embodiments, a Cas protein, e.g., Cas9, comprising mutations in a nuclease domain has reduced (e.g., nickase) or abolished nuclease activity while maintaining its ability to target a nucleic acid locus at a search target sequence when complexed with a guide nucleic acid, e.g., a PERNA.

In some embodiments, a prime editor comprises a Cas nickase that can bind to the target gene in a sequence-specific manner and generate a single-strand break at a protospacer within double-stranded DNA in the target gene, but not a double-strand break. For example, the Cas nickase can cleave the edit strand (i.e., the PAM strand) or the non-edit strand of the target gene, but may not cleave both. In some embodiments, a prime editor comprises a Cas nickase comprising two nuclease domains (e.g., Cas9), with one of the two nuclease domains modified to lack catalytic activity or deleted. In some embodiments, the Cas nickase of a prime editor comprises a nuclease inactive RuvC domain and a nuclease active HNH domain. In some embodiments, the Cas nickase of a prime editor comprises a nuclease inactive HNH domain and a nuclease active RuvC domain. In some embodiments, a prime editor comprises a Cas9 nickase having an amino acid substitution in the RuvC domain, e.g., an amino acid substitution that reduces or abolishes nuclease activity of the RuvC domain. In some embodiments, the Cas9 nickase comprises a D10X amino acid substitution compared to a wild-type S. pyogenes Cas9, wherein X is any amino acid other than D. In some embodiments, a prime editor comprises a Cas9 nickase having an amino acid substitution in the HNH domain, e.g., an amino acid substitution that reduces or abolishes nuclease activity of the HNH domain. In some embodiments, the Cas9 nickase comprises a H840X amino acid substitution compared to a wild-type S. pyogenes Cas9, wherein X is any amino acid other than H.

In some embodiments, a prime editor comprises a Cas protein that can bind to the target gene in a sequence-specific manner but lacks or has abolished nuclease activity and may not cleave either strand of a double-stranded DNA in a target gene. Abolished activity or lacking activity can refer to an enzymatic activity less than 1%, less than 2%, less than 3%, less than 4%, less than 5%, less than 6%, less than 7%, less than 8%, less than 9%, or less than 10% activity compared to a wild-type exemplary activity (e.g., wild-type Cas9 nuclease activity). In some embodiments, a Cas protein of a prime editor completely lacks nuclease activity. A nuclease, e.g., Cas9, that lacks nuclease activity may be referred to as nuclease inactive or “nuclease dead” (abbreviated by “d”). A nuclease dead Cas protein (e.g., dCas, dCas9) can bind to a target polynucleotide but may not cleave the target polynucleotide. In some embodiments, a dead Cas protein is a dead Cas9 protein. In some embodiments, a prime editor comprises a nuclease dead Cas protein wherein all of the nuclease domains (e.g., both RuvC and HNH nuclease domains in a Cas9 protein; RuvC nuclease domain in a Cpf1 protein) are mutated to lack catalytic activity, or are deleted.

A Cas protein can be modified. A Cas protein, e.g., Cas9, can be modified to increase or decrease nucleic acid binding affinity, nucleic acid binding specificity, and/or enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of the Cas protein.

A Cas protein can be a fusion protein. For example, a Cas protein can be fused to a cleavage domain, an epigenetic modification domain, a transcriptional regulation domain, or a polymerase domain. A Cas protein can also be fused to a heterologous polypeptide providing increased or decreased stability. The fused domain or heterologous polypeptide can be located at the N-terminus, the C-terminus, or internally within the Cas protein.

In some embodiments, the Cas protein of a prime editor is a Class 2 Cas protein. In some embodiments, the Cas protein is a type II Cas protein. In some embodiments, the Cas protein is a Cas9 protein, a modified version of a Cas9 protein, a Cas9 protein homolog, mutant, variant, or a functional fragment thereof. As used herein, a Cas9, Cas9 protein, Cas9 polypeptide or a Cas9 nuclease refers to an RNA guided nuclease comprising one or more Cas9 nuclease domains and a Cas9 gRNA binding domain having the ability to bind a guide polynucleotide, e.g., a PERNA. A Cas9 protein may refer to a wild-type Cas9 protein from any organism or a homolog, ortholog, or paralog from any organisms; any functional mutants or functional variants thereof; or any functional fragments or domains thereof. In some embodiments, a prime editor comprises a full-length Cas9 protein. In some embodiments, the Cas9 protein can generally comprises at least about 50%, 60%, 70%, 80%, 90%, 100% sequence identity to a wild-type reference Cas9 protein (e.g., Cas9 from S. pyogenes). In some embodiments, the Cas9 comprises an amino acid change such as a deletion, insertion, substitution, fusion, chimera, or any combination thereof as compared to a wild-type reference Cas9 protein.

In some embodiments, a Cas9 protein may comprise a Cas9 protein from Streptococcus pyogenes (Sp), Staphylococcus aureus (Sa), Streptococcus canis (Sc), Streptococcus thermophilus (St), Staphylococcus lugdunensis (Slu), Neisseria meningitidis (Nm), Campylobacter jejuni (Cj), Francisella novicida (Fn), or Treponema denticola (Td), or any Cas9 homolog or ortholog from an organism known in the art. In some embodiments, a Cas9 polypeptide is a SpCas9 polypeptide e.g., comprising an amino acid sequence as set forth in NCBI Accession No. WP_038431314 or a fragment or variant thereof. In some embodiments, a Cas9 polypeptide is a SaCas9 polypeptide, e.g., comprising an amino acid sequence as set forth in Uniprot Accession No. J7RUA5 or a fragment or variant thereof. In some embodiments, a Cas9 polypeptide is a ScCas9 polypeptide, e.g., comprising an amino acid sequence as set forth in Uniprot Accession No. A0A3P5YA78 or a fragment or variant thereof. In some embodiments, a Cas9 polypeptide is a StCas9 polypeptide, e.g., comprising an amino acid sequence as set forth in NCBI Accession No. WP_007896501.1 or a fragment or variant thereof. In some embodiments, a Cas9 polypeptide is a SluCas9 polypeptide, e.g., comprising an amino acid sequence as set forth in any of NCBI Accession No. WP_230580236.1 or WP_250638315.1 or WP_242234150.1, WP_241435384.1, WP_002460848.1, KAK58371.1, or a fragment or variant thereof. In some embodiments, a Cas9 polypeptide is a NmCas9 polypeptide, e.g., comprising an amino acid sequence as set forth in any of NCBI Accession No. WP_002238326.1 or WP_061704949.1 or a fragment or variant thereof. In some embodiments, a Cas9 polypeptide is a CjCas9 polypeptide, e.g., comprising an amino acid sequence as set forth in any of NCBI Accession No. WP_100612036.1, WP_116882154.1, WP_116560509.1, WP_116484194.1, WP_116479303.1, WP_115794652.1, WP_100624872.1, or a fragment or variant thereof. In some embodiments, a Cas9 polypeptide is a FnCas9 polypeptide, e.g., comprising the amino acid sequence as set forth in Uniprot Accession No. A0Q5Y3 or a fragment or variant thereof. In some embodiments, a Cas9 polypeptide is a TdCas9 polypeptide, e.g., comprising the amino acid sequence as set forth in NCBI Accession No. WP_147625065.1 or a fragment or variant thereof. In some embodiments, a Cas9 polypeptide is a chimera comprising domains from two or more of the organisms described herein or those known in the art. In some embodiments, a Cas9 polypeptide is a Cas9 polypeptide from Streptococcus macacae, e.g., comprising the amino acid sequence as set forth in NCBI Accession No. WP_003079701.1 or a fragment or variant thereof. In some embodiments, a Cas9 polypeptide is a Cas9 polypeptide generated by replacing a PAM interaction domain of a SpCas9 with that of a Streptococcus macacae Cas9 (Spy-mac Cas9). Exemplary Cas9 and Cas9 nickase variants are provided in Table 1B.

In some embodiments, a prime editor comprises a DNA binding domain that comprises an amino acid sequence that is at least 50%, at least 60%, at least 70%, at least 75%, at least 80%, 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of the sequences set forth in Table 1B. In some embodiments, the DNA binding domain comprises an amino acid sequence that has no more than 1, 2, 3, 4, 5, 6, 7, 8, 9, 10, 11, 12, 13, 14, 15, 16, 17, 18, 19, or 20 differences e.g., mutations e.g., deletions, substitutions and/or insertions compared to any one of the amino acid sequences set forth in Table 1B.

In some embodiments, a prime editor comprises a Cas9 protein that comprises an amino acid sequence that is at least 50%, at least 60%, at least 70%, at least 75%, at least 80%, 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of the sequences set forth in Table 1B. In some embodiments, a prime editor comprises a Cas9 protein is a Cas9 nickase that comprises an amino acid sequence that is at least 50%, at least 60%, at least 70%, at least 75%, at least 80%, 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of the nickase sequences set forth in Table 1B. In some embodiments, a Cas9 protein comprises an amino acid sequence that is selected from the group consisting of the sequences set forth in Table 1B. In some embodiments, a prime editor comprises a Cas9 protein that comprises an amino acid sequence that lacks a N-terminus methionine relative to an amino acid sequence set forth Table 1B. In some embodiments, the prime editing compositions or prime editing systems disclosed herein comprises a polynucleotide (e.g., a DNA, or an RNA, e.g., an mRNA) that encodes a Cas9 protein that comprises an amino acid sequence that is at least 50%, at least 60%, at least 70%, at least 75%, at least 80%, 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of the sequences set forth in Table 1B.

In some embodiments, a Cas9 protein comprises a Cas9 protein from Streptococcus pyogenes (Sp), e.g., as according to NC_002737.2:854751-858857 or the protein encoded by UniProt Q99ZW2, e.g., as according to SEQ ID NO: 1005. In some embodiments, a prime editor comprises a Cas9 protein (e.g., a SpCas9) as according to any one of the sequences set forth in SEQ ID NOs: 1005-1008 or a variant thereof. In some embodiments, the Cas9 protein is a SpCas9. In some embodiments, a SpCas9 can be a wild type SpCas9, a SpCas9 variant, or a nickase SpCas9. In some embodiments, the SpCas9 lacks the N-terminus methionine relative to a corresponding SpCas9 (e.g., a wild type SpCas9, a SpCas9 variant or a nickase SpCas9). In some embodiments, a prime editor comprises a Cas9 protein having an amino acid sequence as according to SEQ ID NO: 1005, not including the N-terminus methionine. In some embodiments, a wild type SpCas9 comprises an amino acid sequence set forth in SEQ ID NO: 1005. In some embodiments, a prime editor comprises a Cas9 protein comprising one or more mutations (e.g., amino acid substitutions, insertions and/or deletions) relative to a corresponding wild type Cas9 protein (e.g., a wild type SpCas9). In some embodiments, the Cas9 protein comprising one or more mutations relative to a wild type Cas9 (e.g., a wild type SpCas9) protein comprises an amino acid sequence set forth in SEQ ID NOs: 1006, 1007, or 1008. Exemplary Streptococcus pyogenes Cas9 (SpCas9) amino acid sequence useful in the prime editors disclosed herein are provided in Table 1B.

In some embodiments, a prime editor comprises a Cas9 protein (e.g., a SluCas9) as according to any one of the SEQ ID NOs: 1009-1011 or a variant thereof. In some embodiments, a prime editor comprises a Cas9 protein from Staphylococcus lugdunensis (SluCas9) e.g., as according to any one of the SEQ ID NOs: 1009-1011 or a variant thereof. In some embodiments, the Cas9 protein is a SluCas9. In some embodiments, a SluCas9 can be a wild type SluCas9, a SluCas9 variant, or a nickase SluCas9. In some embodiments, the SluCas9 lacks the N-terminus methionine relative to a corresponding SluCas9 (e.g., a wild type SluCas9, a SluCas9 variant or a nickase SluCas9). In some embodiments, a prime editor comprises a Cas9 protein, having an amino acid sequence as according to SEQ ID NO: 1009, not including the N-terminus methionine. In some embodiments, a wild type SluCas9 comprises an amino acid sequence set forth in SEQ ID NO: 1009. In some embodiments, a prime editor comprises a Cas9 protein comprising one or more mutations (e.g., amino acid substitutions, insertions and/or deletions) relative to a corresponding wild type Cas9 protein (e.g., a wild type SluCas9). In some embodiments, the Cas9 protein comprising one or mutations relative to a wild type Cas9 protein comprises an amino acid sequence set forth in SEQ ID NO: 1010 or SEQ ID NO: 1011. Exemplary Staphylococcus lugdunensis Cas9 (SluCas9) amino acid sequence useful in the prime editors disclosed herein are provided in Table 1B.

In some embodiments, a prime editor comprises a Cas9 protein from Staphylococcus aureus (SaCas9) e.g., as according to any of the SEQ ID NOs: 1012-1014, or a variant thereof. In some embodiments, a prime editor comprises a Cas9 protein from Staphylococcus aureus (SaCas9) e.g., as according to any one of the SEQ ID NOs: 1012-1014, or a variant thereof. In some embodiments, the Cas9 protein is a SaCas9. In some embodiments, a SaCas9 can be a wild type SaCas9, a SaCas9 variant, or a nickase SaCas9. In some embodiments, the SaCas9 lacks the N-terminus methionine relative to a corresponding SaCas9 (e.g., a wild type SaCas9, a SaCas9 variant or a nickase SaCas9). In some embodiments, a prime editor comprises a Cas9 protein, having an amino acid sequence as according to SEQ ID NO: 1012, not including the N-terminus methionine. In some embodiments, a wild type SaCas9 comprises an amino acid sequence set forth in SEQ ID NO: 1012. In some embodiments, a prime editor comprises a Cas9 protein comprising one or more mutations (e.g., amino acid substitutions, insertions and/or deletions relative to a corresponding wild type Cas9 protein (e.g., a wild type SaCas9). In some embodiments, the Cas9 protein comprising one or more mutations relative to a wild type Cas9 protein comprises an amino acid sequence set forth in SEQ ID NO: 1013 or SEQ ID NO: 1014. Exemplary Staphylococcus aureus Cas9 (SaCas9) amino acid sequence useful in the prime editors disclosed herein are provided Table 1B.

In some embodiments, a prime editor comprises a Cas9 protein as according to any one of the sequences set forth in SEQ ID NOs: 1015-1023, 1030-1032 or a variant thereof. In some embodiments, the Cas9 protein is a Cas9 variant, for example, a SpCas9 variant (e.g., SpCas9-NG, SpCas9-NGA, SpRY, or SpG). In some embodiments, the Cas9 protein lacks the N-terminus methionine relative to a corresponding Cas9 protein (e.g., a Cas9 variant set forth in any one of SEQ ID NOs: 1015, 1016, 1018, 1019, 1021, 1022, 1030, or 1031). In some embodiments, a prime editor comprises a Cas9 protein (e.g., a Cas9 variant), having an amino acid sequence as according to any one of SEQ ID NOs: 1015, 1018, 1021, or 1030 not including the N-terminus methionine. In some embodiments, a prime editor comprises a Cas9 protein comprising one or more mutations (e.g., amino acid substitutions, insertions and/or deletions) relative to a corresponding Cas9 protein (e.g., a Cas9 protein set forth in any one of SEQ ID NOs: 1015, 1018, 1021, or 1030). In some embodiments, the Cas9 protein comprising one or mutations relative to a corresponding Cas9 protein comprises an amino acid sequence set forth in any one of SEQ ID NOs: 1016, 1017, 1019, 1020, 1022, 1023, 1031, or 1032.

In some embodiments, a Cas9 protein is a chimeric Cas9, e.g., modified Cas9, e.g., synthetic RNA-guided nucleases (sRGNs), e.g., modified by DNA family shuffling, e.g., sRGN3.1, sRGN3.3. In some embodiments, the DNA family shuffling comprises, fragmentation and reassembly of parental Cas9 genes, e.g., one or more of Cas9s from Staphylococcus hyicus (Shy), Staphylococcus lugdunensis (Slu), Staphylococcus microti (Smi), and Staphylococcus pasteuri (Spa). In some embodiments, a modified sluCas9 shows increased editing efficiency and/or specificity relative to a sluCas9 that is not modified. In some embodiments, a modified Cas9, e.g., a sRGN shows at least 10%, at least 20%, at least 30%, at least 40%, at least 50%, at least 60%, at least 70%, at least 80%, at least 90%, at least 100%, at least 150%, at least 200%, at least 300%, at least 400%, at least 500%, at least 600%, at least 700%, at least 800%, at least 900%, or at least 1000% increase in editing efficiency compared to a Cas9 that is not modified. In some embodiments, a Cas9, e.g., a sRGN shows at least 10%, at least 20%, at least 30%, at least 40%, at least 50%, at least 60%, at least 70%, at least 80%, at least 90%, at least 100%, at least 150%, at least 200%, at least 300%, at least 400%, at least 500%, at least 600%, at least 700%, at least 800%, at least 900%, or at least 1000% increase in specificity compared to a Cas9 that is not modified. In some embodiments, a Cas9, e.g., a sRGN shows at least 10%, at least 20%, at least 30%, at least 40%, at least 50%, at least 60%, at least 70%, at least 80%, at least 90%, at least 100%, at least 150%, at least 200%, at least 300%, at least 400%, at least 500%, at least 600%, at least 700%, at least 800%, at least 900%, or at least 1000% increase in cleavage activity compared to a Cas9 that is not modified. In some embodiments, a Cas9, e.g., a sRGN shows ability to cleave a 5′-NNGG-3′ PAM-containing target. In some embodiments, a prime editor comprises a Cas9 protein (e.g., a chimeric Cas9), e.g., as according any one of the sequences set forth in SEQ ID NOs: 1024-1029, or a variant thereof. Exemplary amino acid sequences of Cas9 protein (e.g., sRGN) useful in the prime editors disclosed herein are provided below in SEQ ID NOs: 1024-1029. In some embodiments, a prime editor comprises a Cas9 protein, that lacks a N-terminus methionine relative to SEQ ID NO: 1024 or SEQ ID NO: 1027. In some embodiments, a prime editor comprises a Cas9 protein comprising one or more mutations (e.g., amino acid substitutions, insertions and/or deletions) relative to a corresponding Cas9 protein (e.g., a Cas9 protein set forth in SEQ ID NO: 1024 or SEQ ID NO: 1027). In some embodiments, the Cas9 protein comprising one or mutations relative to a corresponding Cas9 protein comprises an amino acid sequence set forth in any one of SEQ ID NOs: 1025, 1026, 1028, or 1029.

In some embodiments, a Cas9 protein comprises a variant Cas9 protein containing one or more amino acid substitutions. In some embodiments, a wild-type Cas9 protein comprises a RuvC domain and an HNH domain. In some embodiments, a prime editor comprises a nuclease active Cas9 protein that may cleave both strands of a double-stranded target DNA sequence. In some embodiments, the nuclease active Cas9 protein comprises a functional RuvC domain and a functional HNH domain. In some embodiments, a prime editor comprises a Cas9 nickase that can bind to a guide polynucleotide and recognize a target DNA, but can cleave only one strand of a double-stranded target DNA. In some embodiments, the Cas9 nickase comprises only one functional RuvC domain or one functional HNH domain. In some embodiments, a prime editor comprises a Cas9 that has a non-functional HNH domain and a functional RuvC domain. In some embodiments, the prime editor can cleave the edit strand (i.e., the PAM strand), but not the non-edit strand of a double-stranded target DNA sequence. In some embodiments, a prime editor comprises a Cas9 having a non-functional RuvC domain that can cleave the target strand (i.e., the non-PAM strand), but not the edit strand of a double-stranded target DNA sequence. In some embodiments, a prime editor comprises a Cas9 that has neither a functional RuvC domain nor a functional HNH domain, which may not cleave any strand of a double-stranded target DNA sequence.

In some embodiments, a prime editor comprises a Cas9 having a mutation in the RuvC domain that reduces or abolishes the nuclease activity of the RuvC domain. In some embodiments, the Cas9 comprise a mutation at amino acid D10 as compared to a wild-type SpCas9 as set forth in SEQ ID NO: 1005, or a corresponding mutation thereof. In some embodiments, the Cas9 comprise a D10A mutation as compared to a wild-type SpCas9 as set forth in SEQ ID NO: 1005, or a corresponding mutation thereof. In some embodiments, the Cas9 polypeptide comprise a mutation at amino acid D10, G12, and/or G17 as compared to a wild-type SpCas9 as set forth in SEQ ID NO: 1005, or a corresponding mutation thereof. In some embodiments, the Cas9 polypeptide comprise a D10A mutation, a G12A mutation, and/or a G17A mutation as compared to a wild-type SpCas9 as set forth in SEQ ID NO: 1005, or a corresponding mutation thereof.

In some embodiments, a prime editor comprises a Cas9 polypeptide having a mutation in the HNH domain that reduces or abolishes the nuclease activity of the HNH domain. In some embodiments, the Cas9 polypeptide comprise a mutation at amino acid H840 as compared to a wild-type SpCas9 as set forth in SEQ ID NO: 1005, or a corresponding mutation thereof. In some embodiments, the Cas9 polypeptide comprise a H840A mutation as compared to a wild-type SpCas9 as set forth in SEQ ID NO: 1005, or a corresponding mutation thereof. In some embodiments, the Cas9 polypeptide comprises a mutation at amino acid E762, D839, H840, N854, N856, N863, H982, H983, A984, D986, and/or a A987 as compared to a wild-type SpCas9 as set forth in SEQ ID NO: 1005, or a corresponding mutation thereof. In some embodiments, the Cas9 polypeptide comprise a E762A, D839A, H840A, N854A, N856A, N863A, H982A, H983A, A984A, and/or a D986A mutation as compared to a wild-type SpCas9 as set forth in SEQ ID NO: 1005, or a corresponding mutation thereof. In some embodiments, the Cas9 polypeptide comprises a mutation at amino acid residue R221, N394, and/or H840 as compared to a wild type SpCas9 (e.g., SEQ ID NO: 1005). In some embodiments, the Cas9 polypeptide comprises a R221K, N394L, and/or H840A mutation as compared to a wild type SpCas9 as set forth in SEQ ID NO: 1005, or a corresponding mutation thereof. In some embodiments, the Cas9 polypeptide comprises a mutation at amino acid residue R220, N393, and/or H839 as compared to a wild type SpCas9 (e.g., SEQ ID NO: 1005) lacking a N-terminal methionine, or a corresponding mutation thereof. In some embodiments, the Cas9 polypeptide comprises a R220K, N393K, and/or H839A mutation as compared to a wild type SpCas9 (as set forth in SEQ ID NO: 1005) lacking a N-terminal methionine, or a corresponding mutation thereof.

In some embodiments, a prime editor comprises a Cas9 having one or more amino acid substitutions in both the HNH domain and the RuvC domain that reduce or abolish the nuclease activity of both the HNH domain and the RuvC domain. In some embodiments, the prime editor comprises a nuclease inactive Cas9, or a nuclease dead Cas9 (dCas9). In some embodiments, the dCas9 comprises a H840X substitution and a D10X mutation compared to a wild-type SpCas9 as set forth in SEQ ID NO: 1005 or corresponding mutations thereof, wherein X is any amino acid other than H for the H840X substitution and any amino acid other than D for the D10X substitution. In some embodiments, the dead Cas9 comprises a H840A and a D10A mutation as compared to a wild-type SpCas9 as set forth in SEQ ID NO: 1005, or corresponding mutations thereof.

In some embodiments, the N-terminal methionine is removed from a Cas9 nickase, or from any Cas9 variant, ortholog, or equivalent disclosed or contemplated herein. For example, methionine-minus (Met (−)) Cas9 nickases include any one of the sequences set forth in SEQ ID NOs: 1007, 1008, 1011, 1014, 1017, 1020, 1023, 1026, 1029, 1032, or a variant thereof having an amino acid sequence that has at least 80%, at least 85%, at least 90%, at least 95%, or at least 99% sequence identity thereto.

Besides dead Cas9 and Cas9 nickase variants, the Cas9 proteins used herein may also include other Cas9 variants having at least about 70% identity, at least about 80% identity, at least about 90% identity, at least about 95% identity, at least about 96% identity, at least about 97% identity, at least about 98% identity, at least about 99% identity, at least about 99.5% identity, or at least about 99.9% sequence identity to any reference Cas9 protein, including any wild-type Cas9, or mutant Cas9 (e.g., a dead Cas9 or Cas9 nickase), or fragment Cas9, or circular permutant Cas9, or other variant of Cas9 disclosed herein or known in the art. In some embodiments, a Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to a reference Cas9, e.g., a wild-type Cas9. In some embodiments, the Cas9 variant comprises a fragment of a reference Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of the reference Cas9, e.g., a wild-type Cas9. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild-type Cas9.

In some embodiments, a Cas9 fragment is a functional fragment that retains one or more Cas9 activities. In some embodiments, the Cas9 fragment is at least 100 amino acids in length. In some embodiments, the fragment is at least 100, 150, 200, 250, 300, 350, 400, 450, 500, 550, 600, 650, 700, 750, 800, 850, 900, 950, 1000, 1050, 1100, 1150, 1200, 1250, or at least 1300 amino acids in length.

Exemplary wild-type Cas proteins and corresponding nickases are provided in Table 1B below.

In some embodiments, a prime editor comprises a Cas protein, e.g., Cas9, containing modifications that allow altered PAM recognition. In prime editing using a Cas-protein-based prime editor, a “protospacer adjacent motif” (PAM), PAM sequence, or PAM-like motif, may be used to refer to a short DNA sequence immediately following the protospacer on the PAM strand of the target gene. In some embodiments, the PAM is recognized by the Cas nuclease in the prime editor during prime editing. In certain embodiments, the PAM is required for target binding of the Cas protein. The specific PAM sequence required for Cas protein recognition may depend on the specific type of the Cas protein. A PAM can be 1, 2, 3, 4, 5, 6, 7, 8, 9, 10 or more nucleotides in length. In some embodiments, a PAM is between 2-6 nucleotides in length. In some embodiments, the PAM can be a 5′ PAM (i.e., located upstream of the 5′ end of the protospacer). In other embodiments, the PAM can be a 3′ PAM (i.e., located downstream of the 5′ end of the protospacer). In some embodiments, the Cas protein of a prime editor recognizes a canonical PAM, for example, a SpCas9 recognizes 5′-NGG-3′ PAM. In some embodiments, the Cas protein of a prime editor has altered or non-canonical PAM specificities. Exemplary PAM sequences and corresponding Cas variants are described in Table 1C below. It should be appreciated that for each of the variants provided, the Cas protein comprises one or more of the amino acid substitutions as indicated compared to a wild-type Cas protein sequence, for example, the Cas9 as set forth in SEQ ID NO: 1005. The PAM motifs as shown in Table 1C below are in the order of 5′ to 3′. In some embodiments, the Cas proteins of the disclosure can also be used to direct transcriptional control of target sequences, for example silencing transcription by sequence-specific binding to target sequences. In some embodiments, a Cas protein described herein may have one or mutations in a PAM recognition motif. In some embodiments, a Cas protein described herein may have altered PAM specificity.

The nucleotides listed in Table 1C are represented by the base codes as provided in the Handbook on Industrial Property Information and Documentation, World Intellectual Property Organization (WIPO) Standard ST.26, Version 1.4. For example, an “R” in Table 1C represents the nucleotide A or G, a “W” in Table 1C represents A or T, a “V” refers to any one of nucleotides A, G, or C, and an “N” refers to any one of nucleotides A, G, C, or T.

TABLE 1B
Exemplary Cas protein sequences

| SEQ ID | Sequence | |
|---|---|---|
| NO: | description | Amino acid sequence |
| 1005 | wild type | MDKKYSIGLDIGTNSVGWAVITDEYKVPSKKFKVLGNTDRHSIKKNLIG |
| | Streptococcus | ALLFDSGETAEATRLKRTARRRYTRRKNRICYLQEIFSNEMAKVDDSFFH |
| | Pyogenes | RLEESFLVEEDKKHERHPIFGNIVDEVAYHEKYPTIYHLRKKLVDSTDKA |
| | Cas9 | DLRLIYLALAHMIKFRGHFLIEGDLNPDNSDVDKLFIQLVQTYNQLFEEN |
| | (SpCas9) | PINASGVDAKAILSARLSKSRRLENLIAQLPGEKKNGLFGNLIALSLGLTP |
| | | NFKSNFDLAEDAKLQLSKDTYDDDLDNLLAQIGDQYADLFLAAKNLSD |
| | | AILLSDILRVNTEITKAPLSASMIKRYDEHHQDLTLLKALVRQQLPEKYK |
| | | EIFFDQSKNGYAGYIDGGASQEEFYKFIKPILEKMDGTEELLVKLNREDL |
| | | LRKQRTFDNGSIPHQIHLGELHAILRRQEDFYPFLKDNREKIEKILTFRIPY |
| | | YVGPLARGNSRFAWMTRKSEETITPWNFEEVVDKGASAQSFIERMTNFD |
| | | KNLPNEKVLPKHSLLYEYFTVYNELTKVKYVTEGMRKPAFLSGEQKKAI |
| | | VDLLFKTNRKVTVKQLKEDYFKKIECFDSVEISGVEDRFNASLGTYHDLL |
| | | KIIKDKDFLDNEENEDILEDIVLTLTLFEDREMIEERLKTYAHLFDDKVM |
| | | KQLKRRRYTGWGRLSRKLINGIRDKQSGKTILDFLKSDGFANRNFMQLI |
| | | HDDSLTFKEDIQKAQVSGQGDSLHEHIANLAGSPAIKKGILQTVKVVDEL |
| | | VKVMGRHKPENIVIEMARENQTTQKGQKNSRERMKRIEEGIKELGSQIL |
| | | KEHPVENTQLQNEKLYLYYLQNGRDMYVDQELDINRLSDYDVDHIVPQ |
| | | SFLKDDSIDNKVLTRSDKNRGKSDNVPSEEVVKKMKNYWRQLLNAKLI |
| | | TQRKFDNLTKAERGGLSELDKAGFIKRQLVETRQITKHVAQILDSRMNT |
| | | KYDENDKLIREVKVITLKSKLVSDFRKDFQFYKVREINNYHHAHDAYLN |
| | | AVVGTALIKKYPKLESEFVYGDYKVYDVRKMIAKSEQEIGKATAKYFFY |
| | | SNIMNFFKTEITLANGEIRKRPLIETNGETGEIVWDKGRDFATVRKVLSM |
| | | PQVNIVKKTEVQTGGFSKESILPKRNSDKLIARKKDWDPKKYGGFDSPT |
| | | VAYSVLVVAKVEKGKSKKLKSVKELLGITIMERSSFEKNPIDFLEAKGYK |
| | | EVKKDLIIKLPKYSLFELENGRKRMLASAGELQKGNELALPSKYVNFLYL |
| | | ASHYEKLKGSPEDNEQKQLFVEQHKHYLDEIIEQISEFSKRVILADANLD |
| | | KVLSAYNKHRDKPIREQAENIIHLFTLTNLGAPAAFKYFDTTIDRKRYTST |
| | | KEVLDATLIHQSITGLYETRIDLSQLGGD |
| 1006 | SpCas9 | MDKKYSIGLDIGTNSVGWAVITDEYKVPSKKFKVLGNTDRHSIKKNLIG |
| | H840A | ALLFDSGETAEATRLKRTARRRYTRRKNRICYLQEIFSNEMAKVDDSFFH |
| | nickase | RLEESFLVEEDKKHERHPIFGNIVDEVAYHEKYPTIYHLRKKLVDSTDKA |
| | | DLRLIYLALAHMIKFRGHFLIEGDLNPDNSDVDKLFIQLVQTYNQLFEEN |
| | | PINASGVDAKAILSARLSKSRRLENLIAQLPGEKKNGLFGNLIALSLGLTP |
| | | NFKSNFDLAEDAKLQLSKDTYDDDLDNLLAQIGDQYADLFLAAKNLSD |
| | | AILLSDILRVNTEITKAPLSASMIKRYDEHHQDLTLLKALVRQQLPEKYK |
| | | EIFFDQSKNGYAGYIDGGASQEEFYKFIKPILEKMDGTEELLVKLNREDL |
| | | LRKQRTFDNGSIPHQIHLGELHAILRRQEDFYPFLKDNREKIEKILTFRIPY |
| | | YVGPLARGNSRFAWMTRKSEETITPWNFEEVVDKGASAQSFIERMTNFD |
| | | KNLPNEKVLPKHSLLYEYFTVYNELTKVKYVTEGMRKPAFLSGEQKKAI |
| | | VDLLFKTNRKVTVKQLKEDYFKKIECFDSVEISGVEDRFNASLGTYHDLL |
| | | KIIKDKDFLDNEENEDILEDIVLTLTLFEDREMIEERLKTYAHLFDDKVM |
| | | KQLKRRRYTGWGRLSRKLINGIRDKQSGKTILDFLKSDGFANRNFMQLI |
| | | HDDSLTFKEDIQKAQVSGQGDSLHEHIANLAGSPAIKKGILQTVKVVDEL |
| | | VKVMGRHKPENIVIEMARENQTTQKGQKNSRERMKRIEEGIKELGSQIL |
| | | KEHPVENTQLQNEKLYLYYLQNGRDMYVDQELDINRLSDYDVDAIVPQ |
| | | SFLKDDSIDNKVLTRSDKNRGKSDNVPSEEVVKKMKNYWRQLLNAKLI |
| | | TQRKFDNLTKAERGGLSELDKAGFIKRQLVETRQITKHVAQILDSRMNT |
| | | KYDENDKLIREVKVITLKSKLVSDFRKDFQFYKVREINNYHHAHDAYLN |
| | | AVVGTALIKKYPKLESEFVYGDYKVYDVRKMIAKSEQEIGKATAKYFFY |
| | | SNIMNFFKTEITLANGEIRKRPLIETNGETGEIVWDKGRDFATVRKVLSM |
| | | PQVNIVKKTEVQTGGFSKESILPKRNSDKLIARKKDWDPKKYGGFDSPT |
| | | VAYSVLVVAKVEKGKSKKLKSVKELLGITIMERSSFEKNPIDFLEAKGYK |
| | | EVKKDLIIKLPKYSLFELENGRKRMLASAGELQKGNELALPSKYVNFLYL |
| | | ASHYEKLKGSPEDNEQKQLFVEQHKHYLDEIIEQISEFSKRVILADANLD |
| | | KVLSAYNKHRDKPIREQAENIIHLFTLTNLGAPAAFKYFDTTIDRKRYTST |
| | | KEVLDATLIHQSITGLYETRIDLSQLGGD |
| 1007 | Met (−) | DKKYSIGLDIGTNSVGWAVITDEYKVPSKKFKVLGNTDRHSIKKNLIGAL |
| | SpCas9 | LFDSGETAEATRLKRTARRRYTRRKNRICYLQEIFSNEMAKVDDSFFHRL |
| | H840A | EESFLVEEDKKHERHPIFGNIVDEVAYHEKYPTIYHLRKKLVDSTDKADL |
| | nickase | RLIYLALAHMIKFRGHFLIEGDLNPDNSDVDKLFIQLVQTYNQLFEENPIN |
| | | ASGVDAKAILSARLSKSRRLENLIAQLPGEKKNGLFGNLIALSLGLTPNFK |
| | | SNFDLAEDAKLQLSKDTYDDDLDNLLAQIGDQYADLFLAAKNLSDAILL |
| | | SDILRVNTEITKAPLSASMIKRYDEHHQDLTLLKALVRQQLPEKYKEIFFD |
| | | QSKNGYAGYIDGGASQEEFYKFIKPILEKMDGTEELLVKLNREDLLRKQ |
| | | RTFDNGSIPHQIHLGELHAILRRQEDFYPFLKDNREKIEKILTFRIPYYVGP |
| | | LARGNSRFAWMTRKSEETITPWNFEEVVDKGASAQSFIERMTNFDKNLP |
| | | NEKVLPKHSLLYEYFTVYNELTKVKYVTEGMRKPAFLSGEQKKAIVDLL |
| | | FKTNRKVTVKQLKEDYFKKIECFDSVEISGVEDRFNASLGTYHDLLKIIK |
| | | DKDFLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHDDSL |
| | | TFKEDIQKAQVSGQGDSLHEHIANLAGSPAIKKGILQTVKVVDELVKVM |
| | | GRHKPENIVIEMARENQTTQKGQKNSRERMKRIEEGIKELGSQILKEHPV |
| | | ENTQLQNEKLYLYYLQNGRDMYVDQELDINRLSDYDVDAIVPQSFLKD |
| | | DSIDNKVLTRSDKNRGKSDNVPSEEVVKKMKNYWRQLLNAKLITQRKF |
| | | DNLTKAERGGLSELDKAGFIKRQLVETRQITKHVAQILDSRMNTKYDEN |
| | | DKLIREVKVITLKSKLVSDFRKDFQFYKVREINNYHHAHDAYLNAVVGT |
| | | ALIKKYPKLESEFVYGDYKVYDVRKMIAKSEQEIGKATAKYFFYSNIMN |
| | | FFKTEITLANGEIRKRPLIETNGETGEIVWDKGRDFATVRKVLSMPQVNI |
| | | VKKTEVQTGGFSKESILPKRNSDKLIARKKDWDPKKYGGFDSPTVAYSV |
| | | LVVAKVEKGKSKKLKSVKELLGITIMERSSFEKNPIDFLEAKGYKEVKKD |
| | | LIIKLPKYSLFELENGRKRMLASAGELQKGNELALPSKYVNFLYLASHYE |
| | | KLKGSPEDNEQKQLFVEQHKHYLDEIIEQISEFSKRVILADANLDKVLSA |
| | | YNKHRDKPIREQAENIIHLFTLTNLGAPAAFKYFDTTIDRKRYTSTKEVL |
| | | DATLIHQSITGLYETRIDLSQLGGD |
| 1008 | Met (−) CAS9 | DKKYSIGLDIGTNSVGWAVITDEYKVPSKKFKVLGNTDRHSIKKNLIGAL |
| | (R221K | LFDSGETAEATRLKRTARRRYTRRKNRICYLQEIFSNEMAKVDDSFFHRL |
| | N394K | EESFLVEEDKKHERHPIFGNIVDEVAYHEKYPTIYHLRKKLVDSTDKADL |
| | H840A) | RLIYLALAHMIKFRGHFLIEGDLNPDNSDVDKLFIQLVQTYNQLFEENPIN |
| | nickase | ASGVDAKAILSARLSKSRKLENLIAQLPGEKKNGLFGNLIALSLGLTPNF |
| | | KSNFDLAEDAKLQLSKDTYDDDLDNLLAQIGDQYADLFLAAKNLSDAIL |
| | | LSDILRVNTEITKAPLSASMIKRYDEHHQDLTLLKALVRQQLPEKYKEIFF |
| | | DQSKNGYAGYIDGGASQEEFYKFIKPILEKMDGTEELLVKLKREDLLRK |
| | | QRTFDNGSIPHQIHLGELHAILRRQEDFYPFLKDNREKIEKILTFRIPYYVG |
| | | PLARGNSRFAWMTRKSEETITPWNFEEVVDKGASAQSFIERMTNFDKNL |
| | | PNEKVLPKHSLLYEYFTVYNELTKVKYVTEGMRKPAFLSGEQKKAIVDL |
| | | LFKTNRKVTVKQLKEDYFKKIECFDSVEISGVEDRFNASLGTYHDLLKII |
| | | KDKDFLDNEENEDILEDIVLTLTLFEDREMIEERLKTYAHLFDDKVMKQL |
| | | KRRRYTGWGRLSRKLINGIRDKQSGKTILDFLKSDGFANRNFMQLIHDD |
| | | SLTFKEDIQKAQVSGQGDSLHEHIANLAGSPAIKKGILQTVKVVDELVKV |
| | | MGRHKPENIVIEMARENQTTQKGQKNSRERMKRIEEGIKELGSQILKEHP |
| | | VENTQLQNEKLYLYYLQNGRDMYVDQELDINRLSDYDVDAIVPQSFLK |
| | | DDSIDNKVLTRSDKNRGKSDNVPSEEVVKKMKNYWRQLLNAKLITQRK |
| | | FDNLTKAERGGLSELDKAGFIKRQLVETRQITKHVAQILDSRMNTKYDE |
| | | NDKLIREVKVITLKSKLVSDFRKDFQFYKVREINNYHHAHDAYLNAVVG |
| | | TALIKKYPKLESEFVYGDYKVYDVRKMIAKSEQEIGKATAKYFFYSNIM |
| | | NFFKTEITLANGEIRKRPLIETNGETGEIVWDKGRDFATVRKVLSMPQVN |
| | | IVKKTEVQTGGFSKESILPKRNSDKLIARKKDWDPKKYGGFDSPTVAYS |
| | | VLVVAKVEKGKSKKLKSVKELLGITIMERSSFEKNPIDFLEAKGYKEVKK |
| | | DLIIKLPKYSLFELENGRKRMLASAGELQKGNELALPSKYVNFLYLASHY |
| | | EKLKGSPEDNEQKQLFVEQHKHYLDEIIEQISEFSKRVILADANLDKVLS |
| | | AYNKHRDKPIREQAENIIHLFTLTNLGAPAAFKYFDTTIDRKRYTSTKEV |
| | | LDATLIHQSITGLYETRIDLSQLGGD |
| 1009 | wild type | MNQKFILGLDIGITSVGYGLIDYETKNIIDAGVRLFPEANVENNEGRRSKR |
| | Staphylococcus | GSRRLKRRRIHRLERVKKLLEDYNLLDQSQIPQSTNPYAIRVKGLSEALS |
| | lugdunensis | KDELVIALLHIAKRRGIHKIDVIDSNDDVGNELSTKEQLNKNSKLLKDKF |
| | (Slu)Cas9 | VCQIQLERMNEGQVRGEKNRFKTADIIKEIIQLLNVQKNFHQLDENFINK |
| | | YIELVEMRREYFEGPGKGSPYGWEGDPKAWYETLMGHCTYFPDELRSV |
| | | KYAYSADLFNALNDLNNLVIQRDGLSKLEYHEKYHIIENVFKQKKKPTL |
| | | KQIANEINVNPEDIKGYRITKSGKPQFTEFKLYHDLKSVLFDQSILENEDV |
| | | LDQIAEILTIYQDKDSIKSKLTELDILLNEEDKENIAQLTGYTGTHRLSLKC |
| | | IRLVLEEQWYSSRNQMEIFTHLNIKPKKINLTAANKIPKAMIDEFILSPVV |
| | | KRTFGQAINLINKIIEKYGVPEDIIIELARENNSKDKQKFINEMQKKNENT |
| | | RKRINEIIGKYGNQNAKRLVEKIRLHDEQEGKCLYSLESIPLEDLLNNPNH |
| | | YEVDHIIPRSVSFDNSYHNKVLVKQSENSKKSNLTPYQYFNSGKSKLSYN |
| | | QFKQHILNLSKSQDRISKKKKEYLLEERDINKFEVQKEFINRNLVDTRYA |
| | | TRELTNYLKAYFSANNMNVKVKTINGSFTDYLRKVWKFKKERNHGYK |
| | | HHAEDALIIANADFLFKENKKLKAVNSVLEKPEIESKQLDIQVDSEDNYS |
| | | EMFIIPKQVQDIKDFRNFKYSHRVDKKPNRQLINDTLYSTRKKDNSTYIV |
| | | QTIKDIYAKDNTTLKKQFDKSPEKFLMYQHDPRTFEKLEVIMKQYANEK |
| | | NPLAKYHEETGEYLTKYSKKNNGPIVKSLKYIGNKLGSHLDVTHQFKSS |
| | | TKKLVKLSIKPYRFDVYLTDKGYKFITISYLDVLKKDNYYYIPEQKYDKL |
| | | KLGKAIDKNAKFIASFYKNDLIKLDGEIYKIIGVNSDTRNMIELDLPDIRY |
| | | KEYCELNNIKGEPRIKKTIGKKVNSIEKLTTDVLGNVFTNTQYTKPQLLF |
| | | KRGN |
| 1010 | SluCas9 | MNQKFILGLDIGITSVGYGLIDYETKNIIDAGVRLFPEANVENNEGRRSKR |
| | N582A | GSRRLKRRRIHRLERVKKLLEDYNLLDQSQIPQSTNPYAIRVKGLSEALS |
| | nickase | KDELVIALLHIAKRRGIHKIDVIDSNDDVGNELSTKEQLNKNSKLLKDKF |
| | | VCQIQLERMNEGQVRGEKNRFKTADIIKEIIQLLNVQKNFHQLDENFINK |
| | | YIELVEMRREYFEGPGKGSPYGWEGDPKAWYETLMGHCTYFPDELRSV |
| | | KYAYSADLFNALNDLNNLVIQRDGLSKLEYHEKYHIIENVFKQKKKPTL |
| | | KQIANEINVNPEDIKGYRITKSGKPQFTEFKLYHDLKSVLFDQSILENEDV |
| | | LDQIAEILTIYQDKDSIKSKLTELDILLNEEDKENIAQLTGYTGTHRLSLKC |
| | | IRLVLEEQWYSSRNQMEIFTHLNIKPKKINLTAANKIPKAMIDEFILSPVV |
| | | KRTFGQAINLINKIIEKYGVPEDIIIELARENNSKDKQKFINEMQKKNENT |
| | | RKRINEIIGKYGNQNAKRLVEKIRLHDEQEGKCLYSLESIPLEDLLNNPNH |
| | | YEVDHIIPRSVSFDNSYHNKVLVKQSEASKKSNLTPYQYFNSGKSKLSYN |
| | | QFKQHILNLSKSQDRISKKKKEYLLEERDINKFEVQKEFINRNLVDTRYA |
| | | TRELTNYLKAYFSANNMNVKVKTINGSFTDYLRKVWKFKKERNHGYK |
| | | HHAEDALIIANADFLFKENKKLKAVNSVLEKPEIESKQLDIQVDSEDNYS |
| | | EMFIIPKQVQDIKDFRNFKYSHRVDKKPNRQLINDTLYSTRKKDNSTYIV |
| | | QTIKDIYAKDNTTLKKQFDKSPEKFLMYQHDPRTFEKLEVIMKQYANEK |
| | | NPLAKYHEETGEYLTKYSKKNNGPIVKSLKYIGNKLGSHLDVTHQFKSS |
| | | TKKLVKLSIKPYRFDVYLTDKGYKFITISYLDVLKKDNYYYIPEQKYDKL |
| | | KLGKAIDKNAKFIASFYKNDLIKLDGEIYKIIGVNSDTRNMIELDLPDIRY |
| | | KEYCELNNIKGEPRIKKTIGKKVNSIEKLTTDVLGNVFTNTQYTKPQLLF |
| | | KRGN |
| 1011 | Met (−) | NQKFILGLDIGITSVGYGLIDYETKNIIDAGVRLFPEANVENNEGRRSKRG |
| | SluCas9 | SRRLKRRRIHRLERVKKLLEDYNLLDQSQIPQSTNPYAIRVKGLSEALSK |
| | nickase | DELVIALLHIAKRRGIHKIDVIDSNDDVGNELSTKEQLNKNSKLLKDKFV |
| | | CQIQLERMNEGQVRGEKNRFKTADIIKEIIQLLNVQKNFHQLDENFINKYI |
| | | ELVEMRREYFEGPGKGSPYGWEGDPKAWYETLMGHCTYFPDELRSVKY |
| | | AYSADLFNALNDLNNLVIQRDGLSKLEYHEKYHIIENVFKQKKKPTLKQI |
| | | ANEINVNPEDIKGYRITKSGKPQFTEFKLYHDLKSVLFDQSILENEDVLDQ |
| | | IAEILTIYQDKDSIKSKLTELDILLNEEDKENIAQLTGYTGTHRLSLKCIRL |
| | | VLEEQWYSSRNQMEIFTHLNIKPKKINLTAANKIPKAMIDEFILSPVVKRT |
| | | FGQAINLINKIIEKYGVPEDIIIELARENNSKDKQKFINEMQKKNENTRKRI |
| | | NEIIGKYGNQNAKRLVEKIRLHDEQEGKCLYSLESIPLEDLLNNPNHYEV |
| | | DHIIPRSVSFDNSYHNKVLVKQSEASKKSNLTPYQYFNSGKSKLSYNQFK |
| | | QHILNLSKSQDRISKKKKEYLLEERDINKFEVQKEFINRNLVDTRYATRE |
| | | LTNYLKAYFSANNMNVKVKTINGSFTDYLRKVWKFKKERNHGYKHHA |
| | | EDALIIANADFLFKENKKLKAVNSVLEKPEIESKQLDIQVDSEDNYSEMFI |
| | | IPKQVQDIKDFRNFKYSHRVDKKPNRQLINDTLYSTRKKDNSTYIVQTIK |
| | | DIYAKDNTTLKKQFDKSPEKFLMYQHDPRTFEKLEVIMKQYANEKNPLA |
| | | KYHEETGEYLTKYSKKNNGPIVKSLKYIGNKLGSHLDVTHQFKSSTKKL |
| | | VKLSIKPYRFDVYLTDKGYKFITISYLDVLKKDNYYYIPEQKYDKLKLGK |
| | | AIDKNAKFIASFYKNDLIKLDGEIYKIIGVNSDTRNMIELDLPDIRYKEYC |
| | | ELNNIKGEPRIKKTIGKKVNSIEKLTTDVLGNVFTNTQYTKPQLLFKRGN |
| 1012 | Staphylococcus | MKRNYILGLDIGITSVGYGIIDYETRDVIDAGVRLFKEANVENNEGRRSK |
| | aureus Cas9 | RGARRLKRRRRHRIQRVKKLLFDYNLLTDHSELSGINPYEARVKGLSQK |
| | (SaCas9) | LSEEEFSAALLHLAKRRGVHNVNEVEEDTGNELSTKEQISRNSKALEEKY |
| | | VAELQLERLKKDGEVRGSINRFKTSDYVKEAKQLLKVQKAYHQLDQSFI |
| | | DTYIDLLETRRTYYEGPGEGSPFGWKDIKEWYEMLMGHCTYFPEELRSV |
| | | KYAYNADLYNALNDLNNLVITRDENEKLEYYEKFQIIENVFKQKKKPTL |
| | | KQIAKEILVNEEDIKGYRVTSTGKPEFTNLKVYHDIKDITARKEIIENAELL |
| | | DQIAKILTIYQSSEDIQEELTNLNSELTQEEIEQISNLKGYTGTHNLSLKAI |
| | | NLILDELWHTNDNQIAIFNRLKLVPKKVDLSQQKEIPTTLVDDFILSPVVK |
| | | RSFIQSIKVINAIIKKYGLPNDIIIELAREKNSKDAQKMINEMQKRNRQTNE |
| | | RIEEIIRTTGKENAKYLIEKIKLHDMQEGKCLYSLEAIPLEDLLNNPFNYE |
| | | VDHIIPRSVSFDNSFNNKVLVKQEENSKKGNRTPFQYLSSSDSKISYETFK |
| | | KHILNLAKGKGRISKTKKEYLLEERDINRFSVQKDFINRNLVDTRYATRG |
| | | LMNLLRSYFRVNNLDVKVKSINGGFTSFLRRKWKFKKERNKGYKHHAE |
| | | DALIIANADFIFKEWKKLDKAKKVMENQMFEEKQAESMPEIETEQEYKE |
| | | IFITPHQIKHIKDFKDYKYSHRVDKKPNRELINDTLYSTRKDDKGNTLIVN |
| | | NLNGLYDKDNDKLKKLINKSPEKLLMYHHDPQTYQKLKLIMEQYGDEK |
| | | NPLYKYYEETGNYLTKYSKKDNGPVIKKIKYYGNKLNAHLDITDDYPNS |
| | | RNKVVKLSLKPYRFDVYLDNGVYKFVTVKNLDVIKKENYYEVNSKCYE |
| | | EAKKLKKISNQAEFIASFYNNDLIKINGELYRVIGVNNDLLNRIEVNMIDI |
| | | TYREYLENMNDKRPPRIIKTIASKTQSIKKYSTDILGNLYEVKSKKHPQIIK |
| | | KG |
| 1013 | SaCas9 | MKRNYILGLDIGITSVGYGIIDYETRDVIDAGVRLFKEANVENNEGRRSK |
| | N580A | RGARRLKRRRRHRIQRVKKLLFDYNLLTDHSELSGINPYEARVKGLSQK |
| | nickase | LSEEEFSAALLHLAKRRGVHNVNEVEEDTGNELSTKEQISRNSKALEEKY |
| | | VAELQLERLKKDGEVRGSINRFKTSDYVKEAKQLLKVQKAYHQLDQSFI |
| | | DTYIDLLETRRTYYEGPGEGSPFGWKDIKEWYEMLMGHCTYFPEELRSV |
| | | KYAYNADLYNALNDLNNLVITRDENEKLEYYEKFQIIENVFKQKKKPTL |
| | | KQIAKEILVNEEDIKGYRVTSTGKPEFTNLKVYHDIKDITARKEIIENAELL |
| | | DQIAKILTIYQSSEDIQEELTNLNSELTQEEIEQISNLKGYTGTHNLSLKAI |
| | | NLILDELWHTNDNQIAIFNRLKLVPKKVDLSQQKEIPTTLVDDFILSPVVK |
| | | RSFIQSIKVINAIIKKYGLPNDIIIELAREKNSKDAQKMINEMQKRNRQTNE |
| | | RIEEIIRTTGKENAKYLIEKIKLHDMQEGKCLYSLEAIPLEDLLNNPFNYE |
| | | VDHIIPRSVSFDNSFNNKVLVKQEEASKKGNRTPFQYLSSSDSKISYETFK |
| | | KHILNLAKGKGRISKTKKEYLLEERDINRFSVQKDFINRNLVDTRYATRG |
| | | LMNLLRSYFRVNNLDVKVKSINGGFTSFLRRKWKFKKERNKGYKHHAE |
| | | DALIIANADFIFKEWKKLDKAKKVMENQMFEEKQAESMPEIETEQEYKE |
| | | IFITPHQIKHIKDFKDYKYSHRVDKKPNRELINDTLYSTRKDDKGNTLIVN |
| | | NLNGLYDKDNDKLKKLINKSPEKLLMYHHDPQTYQKLKLIMEQYGDEK |
| | | NPLYKYYEETGNYLTKYSKKDNGPVIKKIKYYGNKLNAHLDITDDYPNS |
| | | RNKVVKLSLKPYRFDVYLDNGVYKFVTVKNLDVIKKENYYEVNSKCYE |
| | | EAKKLKKISNQAEFIASFYNNDLIKINGELYRVIGVNNDLLNRIEVNMIDI |
| | | TYREYLENMNDKRPPRIIKTIASKTQSIKKYSTDILGNLYEVKSKKHPQIIK |
| | | KG |
| 1014 | Met (−) | KRNYILGLDIGITSVGYGIIDYETRDVIDAGVRLFKEANVENNEGRRSKR |
| | SaCas9 | GARRLKRRRRHRIQRVKKLLFDYNLLTDHSELSGINPYEARVKGLSQKL |
| | nickase | SEEEFSAALLHLAKRRGVHNVNEVEEDTGNELSTKEQISRNSKALEEKY |
| | | VAELQLERLKKDGEVRGSINRFKTSDYVKEAKQLLKVQKAYHQLDQSFI |
| | | DTYIDLLETRRTYYEGPGEGSPFGWKDIKEWYEMLMGHCTYFPEELRSV |
| | | KYAYNADLYNALNDLNNLVITRDENEKLEYYEKFQIIENVFKQKKKPTL |
| | | KQIAKEILVNEEDIKGYRVTSTGKPEFTNLKVYHDIKDITARKEIIENAELL |
| | | DQIAKILTIYQSSEDIQEELTNLNSELTQEEIEQISNLKGYTGTHNLSLKAI |
| | | NLILDELWHTNDNQIAIFNRLKLVPKKVDLSQQKEIPTTLVDDFILSPVVK |
| | | RSFIQSIKVINAIIKKYGLPNDIIIELAREKNSKDAQKMINEMQKRNRQTNE |
| | | RIEEIIRTTGKENAKYLIEKIKLHDMQEGKCLYSLEAIPLEDLLNNPFNYE |
| | | VDHIIPRSVSFDNSFNNKVLVKQEEASKKGNRTPFQYLSSSDSKISYETFK |
| | | KHILNLAKGKGRISKTKKEYLLEERDINRFSVQKDFINRNLVDTRYATRG |
| | | LMNLLRSYFRVNNLDVKVKSINGGFTSFLRRKWKFKKERNKGYKHHAE |
| | | DALIIANADFIFKEWKKLDKAKKVMENQMFEEKQAESMPEIETEQEYKE |
| | | IFITPHQIKHIKDFKDYKYSHRVDKKPNRELINDTLYSTRKDDKGNTLIVN |
| | | NLNGLYDKDNDKLKKLINKSPEKLLMYHHDPQTYQKLKLIMEQYGDEK |
| | | NPLYKYYEETGNYLTKYSKKDNGPVIKKIKYYGNKLNAHLDITDDYPNS |
| | | RNKVVKLSLKPYRFDVYLDNGVYKFVTVKNLDVIKKENYYEVNSKCYE |
| | | EAKKLKKISNQAEFIASFYNNDLIKINGELYRVIGVNNDLLNRIEVNMIDI |
| | | TYREYLENMNDKRPPRIIKTIASKTQSIKKYSTDILGNLYEVKSKKHPQIIK |
| | | KG |
| 1015 | SpCas9-NG | MDKKYSIGLDIGTNSVGWAVITDEYKVPSKKFKVLGNTDRHSIKKNLIG |
| | (VRVRFRR) | ALLFDSGETAEATRLKRTARRRYTRRKNRICYLQEIFSNEMAKVDDSFFH |
| | | RLEESFLVEEDKKHERHPIFGNIVDEVAYHEKYPTIYHLRKKLVDSTDKA |
| | | DLRLIYLALAHMIKFRGHFLIEGDLNPDNSDVDKLFIQLVQTYNQLFEEN |
| | | PINASGVDAKAILSARLSKSRRLENLIAQLPGEKKNGLFGNLIALSLGLTP |
| | | NFKSNFDLAEDAKLQLSKDTYDDDLDNLLAQIGDQYADLFLAAKNLSD |
| | | AILLSDILRVNTEITKAPLSASMIKRYDEHHQDLTLLKALVRQQLPEKYK |
| | | EIFFDQSKNGYAGYIDGGASQEEFYKFIKPILEKMDGTEELLVKLNREDL |
| | | LRKQRTFDNGSIPHQIHLGELHAILRRQEDFYPFLKDNREKIEKILTFRIPY |
| | | YVGPLARGNSRFAWMTRKSEETITPWNFEEVVDKGASAQSFIERMTNFD |
| | | KNLPNEKVLPKHSLLYEYFTVYNELTKVKYVTEGMRKPAFLSGEQKKAI |
| | | VDLLFKTNRKVTVKQLKEDYFKKIECFDSVEISGVEDRFNASLGTYHDLL |
| | | KIIKDKDFLDNEENEDILEDIVLTLTLFEDREMIEERLKTYAHLFDDKVM |
| | | KQLKRRRYTGWGRLSRKLINGIRDKQSGKTILDFLKSDGFANRNFMQLI |
| | | HDDSLTFKEDIQKAQVSGQGDSLHEHIANLAGSPAIKKGILQTVKVVDEL |
| | | VKVMGRHKPENIVIEMARENQTTQKGQKNSRERMKRIEEGIKELGSQIL |
| | | KEHPVENTQLQNEKLYLYYLQNGRDMYVDQELDINRLSDYDVDHIVPQ |
| | | SFLKDDSIDNKVLTRSDKNRGKSDNVPSEEVVKKMKNYWRQLLNAKLI |
| | | TQRKFDNLTKAERGGLSELDKAGFIKRQLVETRQITKHVAQILDSRMNT |
| | | KYDENDKLIREVKVITLKSKLVSDFRKDFQFYKVREINNYHHAHDAYLN |
| | | AVVGTALIKKYPKLESEFVYGDYKVYDVRKMIAKSEQEIGKATAKYFFY |
| | | SNIMNFFKTEITLANGEIRKRPLIETNGETGEIVWDKGRDFATVRKVLSM |
| | | PQVNIVKKTEVQTGGFSKESIRPKRNSDKLIARKKDWDPKKYGGFVSPT |
| | | VAYSVLVVAKVEKGKSKKLKSVKELLGITIMERSSFEKNPIDFLEAKGYK |
| | | EVKKDLIIKLPKYSLFELENGRKRMLASARFLQKGNELALPSKYVNFLYL |
| | | ASHYEKLKGSPEDNEQKQLFVEQHKHYLDEIIEQISEFSKRVILADANLD |
| | | KVLSAYNKHRDKPIREQAENIIHLFTLTNLGAPRAFKYFDTTIDRKVYRS |
| | | TKEVLDATLIHQSITGLYETRIDLSQLGGD |
| 1016 | spCas9-NG | MDKKYSIGLDIGTNSVGWAVITDEYKVPSKKFKVLGNTDRHSIKKNLIG |
| | (H840A_ | ALLFDSGETAEATRLKRTARRRYTRRKNRICYLQEIFSNEMAKVDDSFFH |
| | VRVRFRR) | RLEESFLVEEDKKHERHPIFGNIVDEVAYHEKYPTIYHLRKKLVDSTDKA |
| | Nickase | DLRLIYLALAHMIKFRGHFLIEGDLNPDNSDVDKLFIQLVQTYNQLFEEN |
| | | PINASGVDAKAILSARLSKSRRLENLIAQLPGEKKNGLFGNLIALSLGLTP |
| | | NFKSNFDLAEDAKLQLSKDTYDDDLDNLLAQIGDQYADLFLAAKNLSD |
| | | AILLSDILRVNTEITKAPLSASMIKRYDEHHQDLTLLKALVRQQLPEKYK |
| | | EIFFDQSKNGYAGYIDGGASQEEFYKFIKPILEKMDGTEELLVKLNREDL |
| | | LRKQRTFDNGSIPHQIHLGELHAILRRQEDFYPFLKDNREKIEKILTFRIPY |
| | | YVGPLARGNSRFAWMTRKSEETITPWNFEEVVDKGASAQSFIERMTNFD |
| | | KNLPNEKVLPKHSLLYEYFTVYNELTKVKYVTEGMRKPAFLSGEQKKAI |
| | | VDLLFKTNRKVTVKQLKEDYFKKIECFDSVEISGVEDRFNASLGTYHDLL |
| | | KIIKDKDFLDNEENEDILEDIVLTLTLFEDREMIEERLKTYAHLFDDKVM |
| | | KQLKRRRYTGWGRLSRKLINGIRDKQSGKTILDFLKSDGFANRNFMQLI |
| | | HDDSLTFKEDIQKAQVSGQGDSLHEHIANLAGSPAIKKGILQTVKVVDEL |
| | | VKVMGRHKPENIVIEMARENQTTQKGQKNSRERMKRIEEGIKELGSQIL |
| | | KEHPVENTQLQNEKLYLYYLQNGRDMYVDQELDINRLSDYDVDAIVPQ |
| | | SFLKDDSIDNKVLTRSDKNRGKSDNVPSEEVVKKMKNYWRQLLNAKLI |
| | | TQRKFDNLTKAERGGLSELDKAGFIKRQLVETRQITKHVAQILDSRMNT |
| | | KYDENDKLIREVKVITLKSKLVSDFRKDFQFYKVREINNYHHAHDAYLN |
| | | AVVGTALIKKYPKLESEFVYGDYKVYDVRKMIAKSEQEIGKATAKYFFY |
| | | SNIMNFFKTEITLANGEIRKRPLIETNGETGEIVWDKGRDFATVRKVLSM |
| | | PQVNIVKKTEVQTGGFSKESIRPKRNSDKLIARKKDWDPKKYGGFVSPT |
| | | VAYSVLVVAKVEKGKSKKLKSVKELLGITIMERSSFEKNPIDFLEAKGYK |
| | | EVKKDLIIKLPKYSLFELENGRKRMLASARFLQKGNELALPSKYVNFLYL |
| | | ASHYEKLKGSPEDNEQKQLFVEQHKHYLDEIIEQISEFSKRVILADANLD |
| | | KVLSAYNKHRDKPIREQAENIIHLFTLTNLGAPRAFKYFDTTIDRKVYRS |
| | | TKEVLDATLIHQSITGLYETRIDLSQLGGD |
| 1017 | Met (−) | DKKYSIGLDIGTNSVGWAVITDEYKVPSKKFKVLGNTDRHSIKKNLIGAL |
| | SpCas9-NG | LFDSGETAEATRLKRTARRRYTRRKNRICYLQEIFSNEMAKVDDSFFHRL |
| | Nickase | EESFLVEEDKKHERHPIFGNIVDEVAYHEKYPTIYHLRKKLVDSTDKADL |
| | | RLIYLALAHMIKFRGHFLIEGDLNPDNSDVDKLFIQLVQTYNQLFEENPIN |
| | | ASGVDAKAILSARLSKSRRLENLIAQLPGEKKNGLFGNLIALSLGLTPNFK |
| | | SNFDLAEDAKLQLSKDTYDDDLDNLLAQIGDQYADLFLAAKNLSDAILL |
| | | SDILRVNTEITKAPLSASMIKRYDEHHQDLTLLKALVRQQLPEKYKEIFFD |
| | | QSKNGYAGYIDGGASQEEFYKFIKPILEKMDGTEELLVKLNREDLLRKQ |
| | | RTFDNGSIPHQIHLGELHAILRRQEDFYPFLKDNREKIEKILTFRIPYYVGP |
| | | LARGNSRFAWMTRKSEETITPWNFEEVVDKGASAQSFIERMTNFDKNLP |
| | | NEKVLPKHSLLYEYFTVYNELTKVKYVTEGMRKPAFLSGEQKKAIVDLL |
| | | FKTNRKVTVKQLKEDYFKKIECFDSVEISGVEDRFNASLGTYHDLLKIIK |
| | | DKDFLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHDDSL |
| | | TFKEDIQKAQVSGQGDSLHEHIANLAGSPAIKKGILQTVKVVDELVKVM |
| | | GRHKPENIVIEMARENQTTQKGQKNSRERMKRIEEGIKELGSQILKEHPV |
| | | ENTQLQNEKLYLYYLQNGRDMYVDQELDINRLSDYDVDAIVPQSFLKD |
| | | DSIDNKVLTRSDKNRGKSDNVPSEEVVKKMKNYWRQLLNAKLITQRKF |
| | | DNLTKAERGGLSELDKAGFIKRQLVETRQITKHVAQILDSRMNTKYDEN |
| | | DKLIREVKVITLKSKLVSDFRKDFQFYKVREINNYHHAHDAYLNAVVGT |
| | | ALIKKYPKLESEFVYGDYKVYDVRKMIAKSEQEIGKATAKYFFYSNIMN |
| | | FFKTEITLANGEIRKRPLIETNGETGEIVWDKGRDFATVRKVLSMPQVNI |
| | | VKKTEVQTGGFSKESIRPKRNSDKLIARKKDWDPKKYGGFVSPTVAYSV |
| | | LVVAKVEKGKSKKLKSVKELLGITIMERSSFEKNPIDFLEAKGYKEVKKD |
| | | LIIKLPKYSLFELENGRKRMLASARFLQKGNELALPSKYVNFLYLASHYE |
| | | KLKGSPEDNEQKQLFVEQHKHYLDEIIEQISEFSKRVILADANLDKVLSA |
| | | YNKHRDKPIREQAENIIHLFTLTNLGAPRAFKYFDTTIDRKVYRSTKEVL |
| | | DATLIHQSITGLYETRIDLSQLGGD |
| 1018 | spCas9-NGA | MDKKYSIGLDIGTNSVGWAVITDEYKVPSKKFKVLGNTDRHSIKKNLIG |
| | (VRQR) | ALLFDSGETAEATRLKRTARRRYTRRKNRICYLQEIFSNEMAKVDDSFFH |
| | | RLEESFLVEEDKKHERHPIFGNIVDEVAYHEKYPTIYHLRKKLVDSTDKA |
| | | DLRLIYLALAHMIKFRGHFLIEGDLNPDNSDVDKLFIQLVQTYNQLFEEN |
| | | PINASGVDAKAILSARLSKSRRLENLIAQLPGEKKNGLFGNLIALSLGLTP |
| | | NFKSNFDLAEDAKLQLSKDTYDDDLDNLLAQIGDQYADLFLAAKNLSD |
| | | AILLSDILRVNTEITKAPLSASMIKRYDEHHQDLTLLKALVRQQLPEKYK |
| | | EIFFDQSKNGYAGYIDGGASQEEFYKFIKPILEKMDGTEELLVKLNREDL |
| | | LRKQRTFDNGSIPHQIHLGELHAILRRQEDFYPFLKDNREKIEKILTFRIPY |
| | | YVGPLARGNSRFAWMTRKSEETITPWNFEEVVDKGASAQSFIERMTNFD |
| | | KNLPNEKVLPKHSLLYEYFTVYNELTKVKYVTEGMRKPAFLSGEQKKAI |
| | | VDLLFKTNRKVTVKQLKEDYFKKIECFDSVEISGVEDRFNASLGTYHDLL |
| | | KIIKDKDFLDNEENEDILEDIVLTLTLFEDREMIEERLKTYAHLFDDKVM |
| | | KQLKRRRYTGWGRLSRKLINGIRDKQSGKTILDFLKSDGFANRNFMQLI |
| | | HDDSLTFKEDIQKAQVSGQGDSLHEHIANLAGSPAIKKGILQTVKVVDEL |
| | | VKVMGRHKPENIVIEMARENQTTQKGQKNSRERMKRIEEGIKELGSQIL |
| | | KEHPVENTQLQNEKLYLYYLQNGRDMYVDQELDINRLSDYDVDHIVPQ |
| | | SFLKDDSIDNKVLTRSDKNRGKSDNVPSEEVVKKMKNYWRQLLNAKLI |
| | | TQRKFDNLTKAERGGLSELDKAGFIKRQLVETRQITKHVAQILDSRMNT |
| | | KYDENDKLIREVKVITLKSKLVSDFRKDFQFYKVREINNYHHAHDAYLN |
| | | AVVGTALIKKYPKLESEFVYGDYKVYDVRKMIAKSEQEIGKATAKYFFY |
| | | SNIMNFFKTEITLANGEIRKRPLIETNGETGEIVWDKGRDFATVRKVLSM |
| | | PQVNIVKKTEVQTGGFSKESILPKRNSDKLIARKKDWDPKKYGGFVSPT |
| | | VAYSVLVVAKVEKGKSKKLKSVKELLGITIMERSSFEKNPIDFLEAKGYK |
| | | EVKKDLIIKLPKYSLFELENGRKRMLASARELQKGNELALPSKYVNFLYL |
| | | ASHYEKLKGSPEDNEQKQLFVEQHKHYLDEIIEQISEFSKRVILADANLD |
| | | KVLSAYNKHRDKPIREQAENIIHLFTLTNLGAPAAFKYFDTTIDRKQYRS |
| | | TKEVLDATLIHQSITGLYETRIDLSQLGGD |
| 1019 | spCas9-NGA | MDKKYSIGLDIGTNSVGWAVITDEYKVPSKKFKVLGNTDRHSIKKNLIG |
| | (H840A_VRQR) | ALLFDSGETAEATRLKRTARRRYTRRKNRICYLQEIFSNEMAKVDDSFFH |
| | Nickase | RLEESFLVEEDKKHERHPIFGNIVDEVAYHEKYPTIYHLRKKLVDSTDKA |
| | | DLRLIYLALAHMIKFRGHFLIEGDLNPDNSDVDKLFIQLVQTYNQLFEEN |
| | | PINASGVDAKAILSARLSKSRRLENLIAQLPGEKKNGLFGNLIALSLGLTP |
| | | NFKSNFDLAEDAKLQLSKDTYDDDLDNLLAQIGDQYADLFLAAKNLSD |
| | | AILLSDILRVNTEITKAPLSASMIKRYDEHHQDLTLLKALVRQQLPEKYK |
| | | EIFFDQSKNGYAGYIDGGASQEEFYKFIKPILEKMDGTEELLVKLNREDL |
| | | LRKQRTFDNGSIPHQIHLGELHAILRRQEDFYPFLKDNREKIEKILTFRIPY |
| | | YVGPLARGNSRFAWMTRKSEETITPWNFEEVVDKGASAQSFIERMTNFD |
| | | KNLPNEKVLPKHSLLYEYFTVYNELTKVKYVTEGMRKPAFLSGEQKKAI |
| | | VDLLFKTNRKVTVKQLKEDYFKKIECFDSVEISGVEDRFNASLGTYHDLL |
| | | KIIKDKDFLDNEENEDILEDIVLTLTLFEDREMIEERLKTYAHLFDDKVM |
| | | KQLKRRRYTGWGRLSRKLINGIRDKQSGKTILDFLKSDGFANRNFMQLI |
| | | HDDSLTFKEDIQKAQVSGQGDSLHEHIANLAGSPAIKKGILQTVKVVDEL |
| | | VKVMGRHKPENIVIEMARENQTTQKGQKNSRERMKRIEEGIKELGSQIL |
| | | KEHPVENTQLQNEKLYLYYLQNGRDMYVDQELDINRLSDYDVDAIVPQ |
| | | SFLKDDSIDNKVLTRSDKNRGKSDNVPSEEVVKKMKNYWRQLLNAKLI |
| | | TQRKFDNLTKAERGGLSELDKAGFIKRQLVETRQITKHVAQILDSRMNT |
| | | KYDENDKLIREVKVITLKSKLVSDFRKDFQFYKVREINNYHHAHDAYLN |
| | | AVVGTALIKKYPKLESEFVYGDYKVYDVRKMIAKSEQEIGKATAKYFFY |
| | | SNIMNFFKTEITLANGEIRKRPLIETNGETGEIVWDKGRDFATVRKVLSM |
| | | PQVNIVKKTEVQTGGFSKESILPKRNSDKLIARKKDWDPKKYGGFVSPT |
| | | VAYSVLVVAKVEKGKSKKLKSVKELLGITIMERSSFEKNPIDFLEAKGYK |
| | | EVKKDLIIKLPKYSLFELENGRKRMLASARELQKGNELALPSKYVNFLYL |
| | | ASHYEKLKGSPEDNEQKQLFVEQHKHYLDEIIEQISEFSKRVILADANLD |
| | | KVLSAYNKHRDKPIREQAENIIHLFTLTNLGAPAAFKYFDTTIDRKQYRS |
| | | TKEVLDATLIHQSITGLYETRIDLSQLGGD |
| 1020 | Met(−) | DKKYSIGLDIGTNSVGWAVITDEYKVPSKKFKVLGNTDRHSIKKNLIGAL |
| | spCas9-NGA | LFDSGETAEATRLKRTARRRYTRRKNRICYLQEIFSNEMAKVDDSFFHRL |
| | Nickase | EESFLVEEDKKHERHPIFGNIVDEVAYHEKYPTIYHLRKKLVDSTDKADL |
| | | RLIYLALAHMIKFRGHFLIEGDLNPDNSDVDKLFIQLVQTYNQLFEENPIN |
| | | ASGVDAKAILSARLSKSRRLENLIAQLPGEKKNGLFGNLIALSLGLTPNFK |
| | | SNFDLAEDAKLQLSKDTYDDDLDNLLAQIGDQYADLFLAAKNLSDAILL |
| | | SDILRVNTEITKAPLSASMIKRYDEHHQDLTLLKALVRQQLPEKYKEIFFD |
| | | QSKNGYAGYIDGGASQEEFYKFIKPILEKMDGTEELLVKLNREDLLRKQ |
| | | RTFDNGSIPHQIHLGELHAILRRQEDFYPFLKDNREKIEKILTFRIPYYVGP |
| | | LARGNSRFAWMTRKSEETITPWNFEEVVDKGASAQSFIERMTNFDKNLP |
| | | NEKVLPKHSLLYEYFTVYNELTKVKYVTEGMRKPAFLSGEQKKAIVDLL |
| | | FKTNRKVTVKQLKEDYFKKIECFDSVEISGVEDRFNASLGTYHDLLKIIK |
| | | DKDFLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHDDSL |
| | | TFKEDIQKAQVSGQGDSLHEHIANLAGSPAIKKGILQTVKVVDELVKVM |
| | | GRHKPENIVIEMARENQTTQKGQKNSRERMKRIEEGIKELGSQILKEHPV |
| | | ENTQLQNEKLYLYYLQNGRDMYVDQELDINRLSDYDVDAIVPQSFLKD |
| | | DSIDNKVLTRSDKNRGKSDNVPSEEVVKKMKNYWRQLLNAKLITQRKF |
| | | DNLTKAERGGLSELDKAGFIKRQLVETRQITKHVAQILDSRMNTKYDEN |
| | | DKLIREVKVITLKSKLVSDFRKDFQFYKVREINNYHHAHDAYLNAVVGT |
| | | ALIKKYPKLESEFVYGDYKVYDVRKMIAKSEQEIGKATAKYFFYSNIMN |
| | | FFKTEITLANGEIRKRPLIETNGETGEIVWDKGRDFATVRKVLSMPQVNI |
| | | VKKTEVQTGGFSKESILPKRNSDKLIARKKDWDPKKYGGFVSPTVAYSV |
| | | LVVAKVEKGKSKKLKSVKELLGITIMERSSFEKNPIDFLEAKGYKEVKKD |
| | | LIIKLPKYSLFELENGRKRMLASARELQKGNELALPSKYVNFLYLASHYE |
| | | KLKGSPEDNEQKQLFVEQHKHYLDEIIEQISEFSKRVILADANLDKVLSA |
| | | YNKHRDKPIREQAENIIHLFTLTNLGAPAAFKYFDTTIDRKQYRSTKEVL |
| | | DATLIHQSITGLYETRIDLSQLGGD |
| 1021 | SpRY Cas9 | MDKKYSIGLDIGTNSVGWAVITDEYKVPSKKFKVLGNTDRHSIKKNLIG |
| | | ALLFDSGETAERTRLKRTARRRYTRRKNRICYLQEIFSNEMAKVDDSFFH |
| | | RLEESFLVEEDKKHERHPIFGNIVDEVAYHEKYPTIYHLRKKLVDSTDKA |
| | | DLRLIYLALAHMIKFRGHFLIEGDLNPDNSDVDKLFIQLVQTYNQLFEEN |
| | | PINASGVDAKAILSARLSKSRRLENLIAQLPGEKKNGLFGNLIALSLGLTP |
| | | NFKSNFDLAEDAKLQLSKDTYDDDLDNLLAQIGDQYADLFLAAKNLSD |
| | | AILLSDILRVNTEITKAPLSASMIKRYDEHHQDLTLLKALVRQQLPEKYK |
| | | EIFFDQSKNGYAGYIDGGASQEEFYKFIKPILEKMDGTEELLVKLNREDL |
| | | LRKQRTFDNGSIPHQIHLGELHAILRRQEDFYPFLKDNREKIEKILTFRIPY |
| | | YVGPLARGNSRFAWMTRKSEETITPWNFEEVVDKGASAQSFIERMTNFD |
| | | KNLPNEKVLPKHSLLYEYFTVYNELTKVKYVTEGMRKPAFLSGEQKKAI |
| | | VDLLFKTNRKVTVKQLKEDYFKKIECFDSVEISGVEDRFNASLGTYHDLL |
| | | KIIKDKDFLDNEENEDILEDIVLTLTLFEDREMIEERLKTYAHLFDDKVM |
| | | KQLKRRRYTGWGRLSRKLINGIRDKQSGKTILDFLKSDGFANRNFMQLI |
| | | HDDSLTFKEDIQKAQVSGQGDSLHEHIANLAGSPAIKKGILQTVKVVDEL |
| | | VKVMGRHKPENIVIEMARENQTTQKGQKNSRERMKRIEEGIKELGSQIL |
| | | KEHPVENTQLQNEKLYLYYLQNGRDMYVDQELDINRLSDYDVDHIVPQ |
| | | SFLKDDSIDNKVLTRSDKNRGKSDNVPSEEVVKKMKNYWRQLLNAKLI |
| | | TQRKFDNLTKAERGGLSELDKAGFIKRQLVETRQITKHVAQILDSRMNT |
| | | KYDENDKLIREVKVITLKSKLVSDFRKDFQFYKVREINNYHHAHDAYLN |
| | | AVVGTALIKKYPKLESEFVYGDYKVYDVRKMIAKSEQEIGKATAKYFFY |
| | | SNIMNFFKTEITLANGEIRKRPLIETNGETGEIVWDKGRDFATVRKVLSM |
| | | PQVNIVKKTEVQTGGFSKESIRPKRNSDKLIARKKDWDPKKYGGFLWPT |
| | | VAYSVLVVAKVEKGKSKKLKSVKELLGITIMERSSFEKNPIDFLEAKGYK |
| | | EVKKDLIIKLPKYSLFELENGRKRMLASAKQLQKGNELALPSKYVNFLY |
| | | LASHYEKLKGSPEDNEQKQLFVEQHKHYLDEIIEQISEFSKRVILADANL |
| | | DKVLSAYNKHRDKPIREQAENIIHLFTLTRLGAPRAFKYFDTTIDPKQYRS |
| | | TKEVLDATLIHQSITGLYETRIDLSQLGGD |
| 1022 | SpRY Cas9 | MDKKYSIGLDIGTNSVGWAVITDEYKVPSKKFKVLGNTDRHSIKKNLIG |
| | (H840A) | ALLFDSGETAERTRLKRTARRRYTRRKNRICYLQEIFSNEMAKVDDSFFH |
| | Nickase | RLEESFLVEEDKKHERHPIFGNIVDEVAYHEKYPTIYHLRKKLVDSTDKA |
| | | DLRLIYLALAHMIKFRGHFLIEGDLNPDNSDVDKLFIQLVQTYNQLFEEN |
| | | PINASGVDAKAILSARLSKSRRLENLIAQLPGEKKNGLFGNLIALSLGLTP |
| | | NFKSNFDLAEDAKLQLSKDTYDDDLDNLLAQIGDQYADLFLAAKNLSD |
| | | AILLSDILRVNTEITKAPLSASMIKRYDEHHQDLTLLKALVRQQLPEKYK |
| | | EIFFDQSKNGYAGYIDGGASQEEFYKFIKPILEKMDGTEELLVKLNREDL |
| | | LRKQRTFDNGSIPHQIHLGELHAILRRQEDFYPFLKDNREKIEKILTFRIPY |
| | | YVGPLARGNSRFAWMTRKSEETITPWNFEEVVDKGASAQSFIERMTNFD |
| | | KNLPNEKVLPKHSLLYEYFTVYNELTKVKYVTEGMRKPAFLSGEQKKAI |
| | | VDLLFKTNRKVTVKQLKEDYFKKIECFDSVEISGVEDRFNASLGTYHDLL |
| | | KIIKDKDFLDNEENEDILEDIVLTLTLFEDREMIEERLKTYAHLFDDKVM |
| | | KQLKRRRYTGWGRLSRKLINGIRDKQSGKTILDFLKSDGFANRNFMQLI |
| | | HDDSLTFKEDIQKAQVSGQGDSLHEHIANLAGSPAIKKGILQTVKVVDEL |
| | | VKVMGRHKPENIVIEMARENQTTQKGQKNSRERMKRIEEGIKELGSQIL |
| | | KEHPVENTQLQNEKLYLYYLQNGRDMYVDQELDINRLSDYDVDAIVPQ |
| | | SFLKDDSIDNKVLTRSDKNRGKSDNVPSEEVVKKMKNYWRQLLNAKLI |
| | | TQRKFDNLTKAERGGLSELDKAGFIKRQLVETRQITKHVAQILDSRMNT |
| | | KYDENDKLIREVKVITLKSKLVSDFRKDFQFYKVREINNYHHAHDAYLN |
| | | AVVGTALIKKYPKLESEFVYGDYKVYDVRKMIAKSEQEIGKATAKYFFY |
| | | SNIMNFFKTEITLANGEIRKRPLIETNGETGEIVWDKGRDFATVRKVLSM |
| | | PQVNIVKKTEVQTGGFSKESIRPKRNSDKLIARKKDWDPKKYGGFLWPT |
| | | VAYSVLVVAKVEKGKSKKLKSVKELLGITIMERSSFEKNPIDFLEAKGYK |
| | | EVKKDLIIKLPKYSLFELENGRKRMLASAKQLQKGNELALPSKYVNFLY |
| | | LASHYEKLKGSPEDNEQKQLFVEQHKHYLDEIIEQISEFSKRVILADANL |
| | | DKVLSAYNKHRDKPIREQAENIIHLFTLTRLGAPRAFKYFDTTIDPKQYRS |
| | | TKEVLDATLIHQSITGLYETRIDLSQLGGD |
| 1023 | Met(−) SpRY | DKKYSIGLDIGTNSVGWAVITDEYKVPSKKFKVLGNTDRHSIKKNLIGAL |
| | Cas9 Nickase | LFDSGETAERTRLKRTARRRYTRRKNRICYLQEIFSNEMAKVDDSFFHRL |
| | | EESFLVEEDKKHERHPIFGNIVDEVAYHEKYPTIYHLRKKLVDSTDKADL |
| | | RLIYLALAHMIKFRGHFLIEGDLNPDNSDVDKLFIQLVQTYNQLFEENPIN |
| | | ASGVDAKAILSARLSKSRRLENLIAQLPGEKKNGLFGNLIALSLGLTPNFK |
| | | SNFDLAEDAKLQLSKDTYDDDLDNLLAQIGDQYADLFLAAKNLSDAILL |
| | | SDILRVNTEITKAPLSASMIKRYDEHHQDLTLLKALVRQQLPEKYKEIFFD |
| | | QSKNGYAGYIDGGASQEEFYKFIKPILEKMDGTEELLVKLNREDLLRKQ |
| | | RTFDNGSIPHQIHLGELHAILRRQEDFYPFLKDNREKIEKILTFRIPYYVGP |
| | | LARGNSRFAWMTRKSEETITPWNFEEVVDKGASAQSFIERMTNFDKNLP |
| | | NEKVLPKHSLLYEYFTVYNELTKVKYVTEGMRKPAFLSGEQKKAIVDLL |
| | | FKTNRKVTVKQLKEDYFKKIECFDSVEISGVEDRFNASLGTYHDLLKIIK |
| | | DKDFLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHDDSL |
| | | TFKEDIQKAQVSGQGDSLHEHIANLAGSPAIKKGILQTVKVVDELVKVM |
| | | GRHKPENIVIEMARENQTTQKGQKNSRERMKRIEEGIKELGSQILKEHPV |
| | | ENTQLQNEKLYLYYLQNGRDMYVDQELDINRLSDYDVDAIVPQSFLKD |
| | | DSIDNKVLTRSDKNRGKSDNVPSEEVVKKMKNYWRQLLNAKLITQRKF |
| | | DNLTKAERGGLSELDKAGFIKRQLVETRQITKHVAQILDSRMNTKYDEN |
| | | DKLIREVKVITLKSKLVSDFRKDFQFYKVREINNYHHAHDAYLNAVVGT |
| | | ALIKKYPKLESEFVYGDYKVYDVRKMIAKSEQEIGKATAKYFFYSNIMN |
| | | FFKTEITLANGEIRKRPLIETNGETGEIVWDKGRDFATVRKVLSMPQVNI |
| | | VKKTEVQTGGFSKESIRPKRNSDKLIARKKDWDPKKYGGFLWPTVAYS |
| | | VLVVAKVEKGKSKKLKSVKELLGITIMERSSFEKNPIDFLEAKGYKEVKK |
| | | DLIIKLPKYSLFELENGRKRMLASAKQLQKGNELALPSKYVNFLYLASH |
| | | YEKLKGSPEDNEQKQLFVEQHKHYLDEIIEQISEFSKRVILADANLDKVL |
| | | SAYNKHRDKPIREQAENIIHLFTLTRLGAPRAFKYFDTTIDPKQYRSTKEV |
| | | LDATLIHQSITGLYETRIDLSQLGGD |
| 1024 | sRGN3.1 | MNQKFILGLDIGITSVGYGLIDYETKNIIDAGVRLFPEANVENNEGRRSKR |
| | | GSRRLKRRRIHRLERVKLLLTEYDLINKEQIPTSNNPYQIRVKGLSEILSK |
| | | DELAIALLHLAKRRGIHNVDVAADKEETASDSLSTKDQINKNAKFLESR |
| | | YVCELQKERLENEGHVRGVENRFLTKDIVREAKKIIDTQMQYYPEIDETF |
| | | KEKYISLVETRREYFEGPGQGSPFGWNGDLKKWYEMLMGHCTYFPQEL |
| | | RSVKYAYSADLFNALNDLNNLIIQRDNSEKLEYHEKYHIIENVFKQKKKP |
| | | TLKQIAKEIGVNPEDIKGYRITKSGTPEFTSFKLFHDLKKVVKDHAILDDI |
| | | DLLNQIAEILTIYQDKDSIVAELGQLEYLMSEADKQSISELTGYTGTHSLS |
| | | LKCMNMIIDELWHSSMNQMEVFTYLNMRPKKYELKGYQRIPTDMIDDA |
| | | ILSPVVKRTFIQSINVINKVIEKYGIPEDIIIELARENNSDDRKKFINNLQKK |
| | | NEATRKRINEIIGQTGNQNAKRIVEKIRLHDQQEGKCLYSLESIPLEDLLN |
| | | NPNHYEVDHIIPRSVSFDNSYHNKVLVKQSENSKKSNLTPYQYFNSGKS |
| | | KLSYNQFKQHILNLSKSQDRISKKKKEYLLEERDINKFEVQKEFINRNLV |
| | | DTRYATRELTNYLKAYFSANNMNVKVKTINGSFTDYLRKVWKFKKERN |
| | | HGYKHHAEDALIIANADFLFKENKKLKAVNSVLEKPEIETKQLDIQVDSE |
| | | DNYSEMFIIPKQVQDIKDFRNFKYSHRVDKKPNRQLINDTLYSTRKKDNS |
| | | TYIVQTIKDIYAKDNTTLKKQFDKSPEKFLMYQHDPRTFEKLEVIMKQY |
| | | ANEKNPLAKYHEETGEYLTKYSKKNNGPIVKSLKYIGNKLGSHLDVTHQ |
| | | FKSSTKKLVKLSIKNYRFDVYLTEKGYKFVTIAYLNVFKKDNYYYIPKD |
| | | KYQELKEKKKIKDTDQFIASFYKNDLIKLNGDLYKIIGVNSDDRNIIELDY |
| | | YDIKYKDYCEINNIKGEPRIKKTIGKKTESIEKFTTDVLGNLYLHSTEKAP |
| | | QLIFKRGL |
| 1025 | sRGN3.1 | MNQKFILGLDIGITSVGYGLIDYETKNIIDAGVRLFPEANVENNEGRRSKR |
| | (N585A) | GSRRLKRRRIHRLERVKLLLTEYDLINKEQIPTSNNPYQIRVKGLSEILSK |
| | Nickase | DELAIALLHLAKRRGIHNVDVAADKEETASDSLSTKDQINKNAKFLESR |
| | | YVCELQKERLENEGHVRGVENRFLTKDIVREAKKIIDTQMQYYPEIDETF |
| | | KEKYISLVETRREYFEGPGQGSPFGWNGDLKKWYEMLMGHCTYFPQEL |
| | | RSVKYAYSADLFNALNDLNNLIIQRDNSEKLEYHEKYHIIENVFKQKKKP |
| | | TLKQIAKEIGVNPEDIKGYRITKSGTPEFTSFKLFHDLKKVVKDHAILDDI |
| | | DLLNQIAEILTIYQDKDSIVAELGQLEYLMSEADKQSISELTGYTGTHSLS |
| | | LKCMNMIIDELWHSSMNQMEVFTYLNMRPKKYELKGYQRIPTDMIDDA |
| | | ILSPVVKRTFIQSINVINKVIEKYGIPEDIIIELARENNSDDRKKFINNLQKK |
| | | NEATRKRINEIIGQTGNQNAKRIVEKIRLHDQQEGKCLYSLESIPLEDLLN |
| | | NPNHYEVDHIIPRSVSFDNSYHNKVLVKQSEASKKSNLTPYQYFNSGKS |
| | | KLSYNQFKQHILNLSKSQDRISKKKKEYLLEERDINKFEVQKEFINRNLV |
| | | DTRYATRELTNYLKAYFSANNMNVKVKTINGSFTDYLRKVWKFKKERN |
| | | HGYKHHAEDALIIANADFLFKENKKLKAVNSVLEKPEIETKQLDIQVDSE |
| | | DNYSEMFIIPKQVQDIKDFRNFKYSHRVDKKPNRQLINDTLYSTRKKDNS |
| | | TYIVQTIKDIYAKDNTTLKKQFDKSPEKFLMYQHDPRTFEKLEVIMKQY |
| | | ANEKNPLAKYHEETGEYLTKYSKKNNGPIVKSLKYIGNKLGSHLDVTHQ |
| | | FKSSTKKLVKLSIKNYRFDVYLTEKGYKFVTIAYLNVFKKDNYYYIPKD |
| | | KYQELKEKKKIKDTDQFIASFYKNDLIKLNGDLYKIIGVNSDDRNIIELDY |
| | | YDIKYKDYCEINNIKGEPRIKKTIGKKTESIEKFTTDVLGNLYLHSTEKAP |
| | | QLIFKRGL |
| 1026 | Met(−) | NQKFILGLDIGITSVGYGLIDYETKNIIDAGVRLFPEANVENNEGRRSKRG |
| | sRGN3.1 | SRRLKRRRIHRLERVKLLLTEYDLINKEQIPTSNNPYQIRVKGLSEILSKDE |
| | (N584A) | LAIALLHLAKRRGIHNVDVAADKEETASDSLSTKDQINKNAKFLESRYV |
| | Nickase | CELQKERLENEGHVRGVENRFLTKDIVREAKKIIDTQMQYYPEIDETFKE |
| | | KYISLVETRREYFEGPGQGSPFGWNGDLKKWYEMLMGHCTYFPQELRS |
| | | VKYAYSADLFNALNDLNNLIIQRDNSEKLEYHEKYHIIENVFKQKKKPTL |
| | | KQIAKEIGVNPEDIKGYRITKSGTPEFTSFKLFHDLKKVVKDHAILDDIDL |
| | | LNQIAEILTIYQDKDSIVAELGQLEYLMSEADKQSISELTGYTGTHSLSLK |
| | | CMNMIIDELWHSSMNQMEVFTYLNMRPKKYELKGYQRIPTDMIDDAILS |
| | | PVVKRTFIQSINVINKVIEKYGIPEDIIIELARENNSDDRKKFINNLQKKNE |
| | | ATRKRINEIIGQTGNQNAKRIVEKIRLHDQQEGKCLYSLESIPLEDLLNNP |
| | | NHYEVDHIIPRSVSFDNSYHNKVLVKQSEASKKSNLTPYQYFNSGKSKLS |
| | | YNQFKQHILNLSKSQDRISKKKKEYLLEERDINKFEVQKEFINRNLVDTR |
| | | YATRELTNYLKAYFSANNMNVKVKTINGSFTDYLRKVWKFKKERNHG |
| | | YKHHAEDALIIANADFLFKENKKLKAVNSVLEKPEIETKQLDIQVDSEDN |
| | | YSEMFIIPKQVQDIKDFRNFKYSHRVDKKPNRQLINDTLYSTRKKDNSTY |
| | | IVQTIKDIYAKDNTTLKKQFDKSPEKFLMYQHDPRTFEKLEVIMKQYAN |
| | | EKNPLAKYHEETGEYLTKYSKKNNGPIVKSLKYIGNKLGSHLDVTHQFK |
| | | SSTKKLVKLSIKNYRFDVYLTEKGYKFVTIAYLNVFKKDNYYYIPKDKY |
| | | QELKEKKKIKDTDQFIASFYKNDLIKLNGDLYKIIGVNSDDRNIIELDYYD |
| | | IKYKDYCEINNIKGEPRIKKTIGKKTESIEKFTTDVLGNLYLHSTEKAPQLI |
| | | FKRGL |
| 1027 | sRGN3.3 | MNQKFILGLDIGITSVGYGLIDYETKNIIDAGVRLFPEANVENNEGRRSKR |
| | | GSRRLKRRRIHRLERVKLLLTEYDLINKEQIPTSNNPYQIRVKGLSEILSK |
| | | DELAIALLHLAKRRGIHNVDVAADKEETASDSLSTKDQINKNAKFLESR |
| | | YVCELQKERLENEGHVRGVENRFLTKDIVREAKKIIDTQMQYYPEIDETF |
| | | KEKYISLVETRREYFEGPGQGSPFGWNGDLKKWYEMLMGHCTYFPQEL |
| | | RSVKYAYSADLFNALNDLNNLIIQRDNSEKLEYHEKYHIIENVFKQKKKP |
| | | TLKQIAKEIGVNPEDIKGYRITKSGTPEFTSFKLFHDLKKVVKDHAILDDI |
| | | DLLNQIAEILTIYQDKDSIVAELGQLEYLMSEADKQSISELTGYTGTHSLS |
| | | LKCMNMIIDELWHSSMNQMEVFTYLNMRPKKYELKGYQRIPTDMIDDA |
| | | ILSPVVKRTFIQSINVINKVIEKYGIPEDIIIELARENNSDDRKKFINNLQKK |
| | | NEATRKRINEIIGQTGNQNAKRIVEKIRLHDQQEGKCLYSLESIPLEDLLN |
| | | NPNHYEVDHIIPRSVSFDNSYHNKVLVKQSENSKKSNLTPYQYFNSGKS |
| | | KLSYNQFKQHILNLSKSQDRISKKKKEYLLEERDINKFEVQKEFINRNLV |
| | | DTRYATRELTSYLKAYFSANNMDVKVKTINGSFTNHLRKVWRFDKYRN |
| | | HGYKHHAEDALIIANADFLFKENKKLQNTNKILEKPTIENNTKKVTVEKE |
| | | EDYNNVFETPKLVEDIKQYRDYKFSHRVDKKPNRQLINDTLYSTRMKDE |
| | | HDYIVQTITDIYGKDNTNLKKQFNKNPEKFLMYQNDPKTFEKLSIIMKQY |
| | | SDEKNPLAKYYEETGEYLTKYSKKNNGPIVKKIKLLGNKVGNHLDVTN |
| | | KYENSTKKLVKLSIKNYRFDVYLTEKGYKFVTIAYLNVFKKDNYYYIPK |
| | | DKYQELKEKKKIKDTDQFIASFYKNDLIKLNGDLYKIIGVNSDDRNIIELD |
| | | YYDIKYKDYCEINNIKGEPRIKKTIGKKTESIEKFTTDVLGNLYLHSTEKA |
| | | PQLIFKRGL |
| 1028 | sRGN3.3 | MNQKFILGLDIGITSVGYGLIDYETKNIIDAGVRLFPEANVENNEGRRSKR |
| | (N585A) | GSRRLKRRRIHRLERVKLLLTEYDLINKEQIPTSNNPYQIRVKGLSEILSK |
| | Nickase | DELAIALLHLAKRRGIHNVDVAADKEETASDSLSTKDQINKNAKFLESR |
| | | YVCELQKERLENEGHVRGVENRFLTKDIVREAKKIIDTQMQYYPEIDETF |
| | | KEKYISLVETRREYFEGPGQGSPFGWNGDLKKWYEMLMGHCTYFPQEL |
| | | RSVKYAYSADLFNALNDLNNLIIQRDNSEKLEYHEKYHIIENVFKQKKKP |
| | | TLKQIAKEIGVNPEDIKGYRITKSGTPEFTSFKLFHDLKKVVKDHAILDDI |
| | | DLLNQIAEILTIYQDKDSIVAELGQLEYLMSEADKQSISELTGYTGTHSLS |
| | | LKCMNMIIDELWHSSMNQMEVFTYLNMRPKKYELKGYQRIPTDMIDDA |
| | | ILSPVVKRTFIQSINVINKVIEKYGIPEDIIIELARENNSDDRKKFINNLQKK |
| | | NEATRKRINEIIGQTGNQNAKRIVEKIRLHDQQEGKCLYSLESIPLEDLLN |
| | | NPNHYEVDHIIPRSVSFDNSYHNKVLVKQSEASKKSNLTPYQYFNSGKS |
| | | KLSYNQFKQHILNLSKSQDRISKKKKEYLLEERDINKFEVQKEFINRNLV |
| | | DTRYATRELTSYLKAYFSANNMDVKVKTINGSFTNHLRKVWRFDKYRN |
| | | HGYKHHAEDALIIANADFLFKENKKLQNTNKILEKPTIENNTKKVTVEKE |
| | | EDYNNVFETPKLVEDIKQYRDYKFSHRVDKKPNRQLINDTLYSTRMKDE |
| | | HDYIVQTITDIYGKDNTNLKKQFNKNPEKFLMYQNDPKTFEKLSIIMKQY |
| | | SDEKNPLAKYYEETGEYLTKYSKKNNGPIVKKIKLLGNKVGNHLDVTN |
| | | KYENSTKKLVKLSIKNYRFDVYLTEKGYKFVTIAYLNVFKKDNYYYIPK |
| | | DKYQELKEKKKIKDTDQFIASFYKNDLIKLNGDLYKIIGVNSDDRNIIELD |
| | | YYDIKYKDYCEINNIKGEPRIKKTIGKKTESIEKFTTDVLGNLYLHSTEKA |
| | | PQLIFKRGL |
| 1029 | Met(−) | NQKFILGLDIGITSVGYGLIDYETKNIIDAGVRLFPEANVENNEGRRSKRG |
| | sRGN3.3 | SRRLKRRRIHRLERVKLLLTEYDLINKEQIPTSNNPYQIRVKGLSEILSKDE |
| | (N584A) | LAIALLHLAKRRGIHNVDVAADKEETASDSLSTKDQINKNAKFLESRYV |
| | Nickase | CELQKERLENEGHVRGVENRFLTKDIVREAKKIIDTQMQYYPEIDETFKE |
| | | KYISLVETRREYFEGPGQGSPFGWNGDLKKWYEMLMGHCTYFPQELRS |
| | | VKYAYSADLFNALNDLNNLIIQRDNSEKLEYHEKYHIIENVFKQKKKPTL |
| | | KQIAKEIGVNPEDIKGYRITKSGTPEFTSFKLFHDLKKVVKDHAILDDIDL |
| | | LNQIAEILTIYQDKDSIVAELGQLEYLMSEADKQSISELTGYTGTHSLSLK |
| | | CMNMIIDELWHSSMNQMEVFTYLNMRPKKYELKGYQRIPTDMIDDAILS |
| | | PVVKRTFIQSINVINKVIEKYGIPEDIIIELARENNSDDRKKFINNLQKKNE |
| | | ATRKRINEIIGQTGNQNAKRIVEKIRLHDQQEGKCLYSLESIPLEDLLNNP |
| | | NHYEVDHIIPRSVSFDNSYHNKVLVKQSEASKKSNLTPYQYFNSGKSKLS |
| | | YNQFKQHILNLSKSQDRISKKKKEYLLEERDINKFEVQKEFINRNLVDTR |
| | | YATRELTSYLKAYFSANNMDVKVKTINGSFTNHLRKVWRFDKYRNHGY |
| | | KHHAEDALIIANADFLFKENKKLQNTNKILEKPTIENNTKKVTVEKEEDY |
| | | NNVFETPKLVEDIKQYRDYKFSHRVDKKPNRQLINDTLYSTRMKDEHDY |
| | | IVQTITDIYGKDNTNLKKQFNKNPEKFLMYQNDPKTFEKLSIIMKQYSDE |
| | | KNPLAKYYEETGEYLTKYSKKNNGPIVKKIKLLGNKVGNHLDVTNKYE |
| | | NSTKKLVKLSIKNYRFDVYLTEKGYKFVTIAYLNVFKKDNYYYIPKDKY |
| | | QELKEKKKIKDTDQFIASFYKNDLIKLNGDLYKIIGVNSDDRNIIELDYYD |
| | | IKYKDYCEINNIKGEPRIKKTIGKKTESIEKFTTDVLGNLYLHSTEKAPQLI |
| | | FKRGL |
| 1030 | SpG | MDKKYSIGLDIGTNSVGWAVITDEYKVPSKKFKVLGNTDRHSIKKNLIG |
| | | ALLFDSGETAEATRLKRTARRRYTRRKNRICYLQEIFSNEMAKVDDSFFH |
| | | RLEESFLVEEDKKHERHPIFGNIVDEVAYHEKYPTIYHLRKKLVDSTDKA |
| | | DLRLIYLALAHMIKFRGHFLIEGDLNPDNSDVDKLFIQLVQTYNQLFEEN |
| | | PINASGVDAKAILSARLSKSRRLENLIAQLPGEKKNGLFGNLIALSLGLTP |
| | | NFKSNFDLAEDAKLQLSKDTYDDDLDNLLAQIGDQYADLFLAAKNLSD |
| | | AILLSDILRVNTEITKAPLSASMIKRYDEHHQDLTLLKALVRQQLPEKYK |
| | | EIFFDQSKNGYAGYIDGGASQEEFYKFIKPILEKMDGTEELLVKLNREDL |
| | | LRKQRTFDNGSIPHQIHLGELHAILRRQEDFYPFLKDNREKIEKILTFRIPY |
| | | YVGPLARGNSRFAWMTRKSEETITPWNFEEVVDKGASAQSFIERMTNFD |
| | | KNLPNEKVLPKHSLLYEYFTVYNELTKVKYVTEGMRKPAFLSGEQKKAI |
| | | VDLLFKTNRKVTVKQLKEDYFKKIECFDSVEISGVEDRFNASLGTYHDLL |
| | | KIIKDKDFLDNEENEDILEDIVLTLTLFEDREMIEERLKTYAHLFDDKVM |
| | | KQLKRRRYTGWGRLSRKLINGIRDKQSGKTILDFLKSDGFANRNFMQLI |
| | | HDDSLTFKEDIQKAQVSGQGDSLHEHIANLAGSPAIKKGILQTVKVVDEL |
| | | VKVMGRHKPENIVIEMARENQTTQKGQKNSRERMKRIEEGIKELGSQIL |
| | | KEHPVENTQLQNEKLYLYYLQNGRDMYVDQELDINRLSDYDVDHIVPQ |
| | | SFLKDDSIDNKVLTRSDKNRGKSDNVPSEEVVKKMKNYWRQLLNAKLI |
| | | TQRKFDNLTKAERGGLSELDKAGFIKRQLVETRQITKHVAQILDSRMNT |
| | | KYDENDKLIREVKVITLKSKLVSDFRKDFQFYKVREINNYHHAHDAYLN |
| | | AVVGTALIKKYPKLESEFVYGDYKVYDVRKMIAKSEQEIGKATAKYFFY |
| | | SNIMNFFKTEITLANGEIRKRPLIETNGETGEIVWDKGRDFATVRKVLSM |
| | | PQVNIVKKTEVQTGGFSKESILPKRNSDKLIARKKDWDPKKYGGFLWPT |
| | | VAYSVLVVAKVEKGKSKKLKSVKELLGITIMERSSFEKNPIDFLEAKGYK |
| | | EVKKDLIIKLPKYSLFELENGRKRMLASAKQLQKGNELALPSKYVNFLY |
| | | LASHYEKLKGSPEDNEQKQLFVEQHKHYLDEIIEQISEFSKRVILADANL |
| | | DKVLSAYNKHRDKPIREQAENIIHLFTLTNLGAPAAFKYFDTTIDRKQYR |
| | | STKEVLDATLIHQSITGLYETRIDLSQLGGD |
| 1031 | SpG (H840A) | MDKKYSIGLDIGTNSVGWAVITDEYKVPSKKFKVLGNTDRHSIKKNLIG |
| | Nickase | ALLFDSGETAEATRLKRTARRRYTRRKNRICYLQEIFSNEMAKVDDSFFH |
| | | RLEESFLVEEDKKHERHPIFGNIVDEVAYHEKYPTIYHLRKKLVDSTDKA |
| | | DLRLIYLALAHMIKFRGHFLIEGDLNPDNSDVDKLFIQLVQTYNQLFEEN |
| | | PINASGVDAKAILSARLSKSRRLENLIAQLPGEKKNGLFGNLIALSLGLTP |
| | | NFKSNFDLAEDAKLQLSKDTYDDDLDNLLAQIGDQYADLFLAAKNLSD |
| | | AILLSDILRVNTEITKAPLSASMIKRYDEHHQDLTLLKALVRQQLPEKYK |
| | | EIFFDQSKNGYAGYIDGGASQEEFYKFIKPILEKMDGTEELLVKLNREDL |
| | | LRKQRTFDNGSIPHQIHLGELHAILRRQEDFYPFLKDNREKIEKILTFRIPY |
| | | YVGPLARGNSRFAWMTRKSEETITPWNFEEVVDKGASAQSFIERMTNFD |
| | | KNLPNEKVLPKHSLLYEYFTVYNELTKVKYVTEGMRKPAFLSGEQKKAI |
| | | VDLLFKTNRKVTVKQLKEDYFKKIECFDSVEISGVEDRFNASLGTYHDLL |
| | | KIIKDKDFLDNEENEDILEDIVLTLTLFEDREMIEERLKTYAHLFDDKVM |
| | | KQLKRRRYTGWGRLSRKLINGIRDKQSGKTILDFLKSDGFANRNFMQLI |
| | | HDDSLTFKEDIQKAQVSGQGDSLHEHIANLAGSPAIKKGILQTVKVVDEL |
| | | VKVMGRHKPENIVIEMARENQTTQKGQKNSRERMKRIEEGIKELGSQIL |
| | | KEHPVENTQLQNEKLYLYYLQNGRDMYVDQELDINRLSDYDVDAIVPQ |
| | | SFLKDDSIDNKVLTRSDKNRGKSDNVPSEEVVKKMKNYWRQLLNAKLI |
| | | TQRKFDNLTKAERGGLSELDKAGFIKRQLVETRQITKHVAQILDSRMNT |
| | | KYDENDKLIREVKVITLKSKLVSDFRKDFQFYKVREINNYHHAHDAYLN |
| | | AVVGTALIKKYPKLESEFVYGDYKVYDVRKMIAKSEQEIGKATAKYFFY |
| | | SNIMNFFKTEITLANGEIRKRPLIETNGETGEIVWDKGRDFATVRKVLSM |
| | | PQVNIVKKTEVQTGGFSKESILPKRNSDKLIARKKDWDPKKYGGFLWPT |
| | | VAYSVLVVAKVEKGKSKKLKSVKELLGITIMERSSFEKNPIDFLEAKGYK |
| | | EVKKDLIIKLPKYSLFELENGRKRMLASAKQLQKGNELALPSKYVNFLY |
| | | LASHYEKLKGSPEDNEQKQLFVEQHKHYLDEIIEQISEFSKRVILADANL |
| | | DKVLSAYNKHRDKPIREQAENIIHLFTLTNLGAPAAFKYFDTTIDRKQYR |
| | | STKEVLDATLIHQSITGLYETRIDLSQLGGD |
| 1032 | Met(−) | DKKYSIGLDIGTNSVGWAVITDEYKVPSKKFKVLGNTDRHSIKKNLIGAL |
| | SpG (H839A) | LFDSGETAEATRLKRTARRRYTRRKNRICYLQEIFSNEMAKVDDSFFHRL |
| | Nickase | EESFLVEEDKKHERHPIFGNIVDEVAYHEKYPTIYHLRKKLVDSTDKADL |
| | | RLIYLALAHMIKFRGHFLIEGDLNPDNSDVDKLFIQLVQTYNQLFEENPIN |
| | | ASGVDAKAILSARLSKSRRLENLIAQLPGEKKNGLFGNLIALSLGLTPNFK |
| | | SNFDLAEDAKLQLSKDTYDDDLDNLLAQIGDQYADLFLAAKNLSDAILL |
| | | SDILRVNTEITKAPLSASMIKRYDEHHQDLTLLKALVRQQLPEKYKEIFFD |
| | | QSKNGYAGYIDGGASQEEFYKFIKPILEKMDGTEELLVKLNREDLLRKQ |
| | | RTFDNGSIPHQIHLGELHAILRRQEDFYPFLKDNREKIEKILTFRIPYYVGP |
| | | LARGNSRFAWMTRKSEETITPWNFEEVVDKGASAQSFIERMTNFDKNLP |
| | | NEKVLPKHSLLYEYFTVYNELTKVKYVTEGMRKPAFLSGEQKKAIVDLL |
| | | FKTNRKVTVKQLKEDYFKKIECFDSVEISGVEDRFNASLGTYHDLLKIIK |
| | | DKDFLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHDDSL |
| | | TFKEDIQKAQVSGQGDSLHEHIANLAGSPAIKKGILQTVKVVDELVKVM |
| | | GRHKPENIVIEMARENQTTQKGQKNSRERMKRIEEGIKELGSQILKEHPV |
| | | ENTQLQNEKLYLYYLQNGRDMYVDQELDINRLSDYDVDAIVPQSFLKD |
| | | DSIDNKVLTRSDKNRGKSDNVPSEEVVKKMKNYWRQLLNAKLITQRKF |
| | | DNLTKAERGGLSELDKAGFIKRQLVETRQITKHVAQILDSRMNTKYDEN |
| | | DKLIREVKVITLKSKLVSDFRKDFQFYKVREINNYHHAHDAYLNAVVGT |
| | | ALIKKYPKLESEFVYGDYKVYDVRKMIAKSEQEIGKATAKYFFYSNIMN |
| | | FFKTEITLANGEIRKRPLIETNGETGEIVWDKGRDFATVRKVLSMPQVNI |
| | | VKKTEVQTGGFSKESILPKRNSDKLIARKKDWDPKKYGGFLWPTVAYSV |
| | | LVVAKVEKGKSKKLKSVKELLGITIMERSSFEKNPIDFLEAKGYKEVKKD |
| | | LIIKLPKYSLFELENGRKRMLASAKQLQKGNELALPSKYVNFLYLASHYE |
| | | KLKGSPEDNEQKQLFVEQHKHYLDEIIEQISEFSKRVILADANLDKVLSA |
| | | YNKHRDKPIREQAENIIHLFTLTNLGAPAAFKYFDTTIDRKQYRSTKEVL |
| | | DATLIHQSITGLYETRIDLSQLGGD |

TABLE 1C
Cas protein variants and corresponding PAM sequences

| Variant | PAM |
|---|---|
| spCas9 (wild-type) | NGG, NGA, NAG, NGNGA |
| spCas9-VRVRFRR | NG |
| R1335V/L1111R/D1135V/G1218R/E1219F/A1322R/T1337R | |
| spCas9-VQR (D1135V/R1335Q/T1337R) | NGA |
| spCas9-EQR (D1135E/R1335Q/T1337R) | NGA |
| spCas9-VRER (D1135V/G1218R/R1335E/T1337R) | NGCG |
| spCas9-VRQR (D1135V, G1218R, R1335Q, T1337R) | NGA |
| Cas9-NG (L1111R, D1135V, G1218R, E1219F, A1322R, | NGN |
| T1337R, R1335V) | |
| SpG Cas9 (D1135L, S1136W, G1218K, E1219Q, R1335Q, | NGN |
| T1337R) | |
| SyRY Cas9 | NRN |
| (A61R, L1111R, N1317R, A1322R, and R1333P) | |
| xCas9 (E480K, E543D, E1219V, K294R, Q1256K, A262T, | NGN |
| S409I, M694I) | |
| SluCa9 | NNGG |
| SRGN1, sRGN2, sRGN4, sRGN3.1, sRGN3.3 | NNGG |
| saCas9 | NNGRRT, NNGRRN |
| saCas9-KKH (E782K, N968K, R1015H) | NNNRRT |
| spCas9-MQKSER (D1135M, S1136Q, G1218K, E1219S, | NGCG/NGCN |
| R1335E, T1337R) | |
| spCas9-LRKIQK (D1135L, S1136R, G1218K, E1219I, | NGTN |
| R1335Q, T1337K) | |
| spCas9-LRVSQK (D1135L, S1136R, G1218V, E1219S, | NGTN |
| R1335Q, T1337K) | |
| spCas9-LRVSQL(D1135L, S1136R, G1218V, E1219S, | NGTN |
| R1335Q, T1337L) | |
| Cpf1 | TTTV |
| Spy-Mac | NAA |
| NmCas9 | NNNNGATT |
| StCas9 | NNAGAAW |
| TdCas9 | NAAAAC |

In some embodiments, a prime editor comprises a Cas9 polypeptide comprising one or more mutations selected from the group consisting of: A61R, L111R, D1135V, R221K, A262T, R324L, N394K, S4091, S4091, E427G, E480K, M495V, N497A, Y515N, K526E, F539S, E543D, R654L, R661A, R661L, R691A, N692A, M694A, M694I, Q695A, H698A, R753G, M763I, K848A, K890N, Q926A, K1003A, R1060A, L1111R, R1114G, D1135E, D1135L, D1135N, S1136W, V1139A, D1180G, G1218K, G1218R, G1218S, E1219Q, E1219V, E1219V, Q1221H, P1249S, E1253K, N1317R, A1320V, P1321S, A1322R, I1322V, D1332G, R1332N, A1332R, R1333K, R1333P, R1335L, R1335Q, R1335V, T1337N, T1337R, S1338T, H1349R, and any combinations thereof as compared to a wild-type SpCas9 polypeptide as set forth in SEQ ID NO: 1005.

In some embodiments, a prime editor comprises a SaCas9 polypeptide. In some embodiments, the SaCas9 polypeptide comprises one or more of mutations E782K, N968K, and R1015H as compared to a wild-type SaCas9. In some embodiments, a prime editor comprises a FnCas9 polypeptide, for example, a wild-type FnCas9 polypeptide or a FnCas9 polypeptide comprising one or more of mutations E1369R, E1449H, or R1556A as compared to the wild-type FnCas9. In some embodiments, a prime editor comprises a Sc Cas9, for example, a wild-type ScCas9 or a ScCas9 polypeptide comprises one or more of mutations 1367K, G368D, 1369K, H371L, T375S, T376G, and T1227K as compared to the wild-type ScCas9. In some embodiments, a prime editor comprises a St1 Cas9 polypeptide, a St3 Cas9 polypeptide, or a Slu Cas9 polypeptide.

In some embodiments, a prime editor comprises a Cas polypeptide that comprises a circular permutant Cas variant. For example, a Cas9 polypeptide of a prime editor may be engineered such that the N-terminus and the C-terminus of a Cas9 protein (e.g., a wild-type Cas9 protein, or a Cas9 nickase) are topically rearranged to retain the ability to bind DNA when complexed with a guide RNA (gRNA). An exemplary circular permutant configuration may be N-terminus-[original C-terminus]-[original N-terminus]-C-terminus. Any of the Cas9 proteins described herein, including any variant, ortholog, or naturally occurring Cas9 or equivalent thereof, may be reconfigured as a circular permutant variant.

In various embodiments, the circular permutants of a Cas protein, e.g., a Cas9, may have the following structure: N-terminus-[original C-terminus]-[optional linker]-[original N-terminus]-C-terminus. In some embodiments, a circular permutant Cas9 comprises any one of the following structures (amino acid positions as set forth in SEQ ID NO: 1005):
N-terminus-[1268-1368]-[optional linker]-[1-1267]-C-terminus;
N-terminus-[1168-1368]-[optional linker]-[1-1167]-C-terminus;
N-terminus-[1068-1368]-[optional linker]-[1-1067]-C-terminus;
N-terminus-[968-1368]-[optional linker]-[1-967]-C-terminus;
N-terminus-[868-1368]-[optional linker]-[1-867]-C-terminus;
N-terminus-[768-1368]-[optional linker]-[1-767]-C-terminus;
N-terminus-[668-1368]-[optional linker]-[1-667]-C-terminus;
N-terminus-[568-1368]-[optional linker]-[1-567]-C-terminus;
N-terminus-[468-1368]-[optional linker]-[1-467]-C-terminus;
N-terminus-[368-1368]-[optional linker]-[1-367]-C-terminus;
N-terminus-[268-1368]-[optional linker]-[1-267]-C-terminus;
N-terminus-[168-1368]-[optional linker]-[1-167]-C-terminus;
N-terminus-[68-1368]-[optional linker]-[1-67]-C-terminus;
N-terminus-[10-1368]-[optional linker]-[1-9]-C-terminus; or the corresponding circular permutants of other Cas9 proteins (including other Cas9 orthologs, variants, etc).

In some embodiments, a circular permutant Cas9 comprises any one of the following structures (amino acid positions as set forth in SEQ ID NO: 1005):
N-terminus-[102-1368]-[optional linker]-[1-101]-C-terminus;
N-terminus-[1028-1368]-[optional linker]-[1-1027]-C-terminus;
N-terminus-[1041-1368]-[optional linker]-[1-1043]-C-terminus;
N-terminus-[1249-1368]-[optional linker]-[1-1248]-C-terminus;
N-terminus-[1300-1368]-[optional linker]-[1-1299]-C-terminus; or the corresponding circular permutants of other Cas9 proteins (including other Cas9 orthologs, variants, etc).

In some embodiments, a circular permutant Cas9 comprises any one of the following structures (amino acid positions as set forth in SEQ ID NO: 1005):
N-terminus-[103-1368]-[optional linker]-[1-102]-C-terminus;
N-terminus-[1029-1368]-[optional linker]-[1-1028]-C-terminus;
N-terminus-[1042-1368]-[optional linker]-[1-1041]-C-terminus;
N-terminus-[1250-1368]-[optional linker]-[1-1249]-C-terminus;
N-terminus-[1301-1368]-[optional linker]-[1-1300]-C-terminus; or the corresponding circular permutants of other Cas9 proteins (including other Cas9 orthologs, variants, etc).

In some embodiments, the circular permutant can be formed by linking a C-terminal fragment of a Cas9 to an N-terminal fragment of a Cas9, either directly or by using a linker, such as an amino acid linker. In some embodiments, the C-terminal fragment may correspond to the 95% or more of the C-terminal amino acids of a Cas9 (e.g., amino acids about 1300-1368 as set forth in SEQ ID NO: 1005 or corresponding amino acid positions thereof), or the 90%, 85%, 80%, 75%, 70%, 65%, 60%, 55%, 50%, 45%, 40%, 35%, 30%, 25%, 20%, 15%, 10%, or 5% or more of the C-terminal amino acids of a Cas9 (e.g., SEQ ID NO: 1005 or a ortholog or a variant thereof). The N-terminal portion may correspond to 95% or more of the N-terminal amino acids of (e.g., amino acids about 1-1300 as set forth in SEQ ID NO: 1005 or corresponding amino acid positions thereof), or 90%, 85%, 80%, 75%, 70%, 65%, 60%, 55%, 50%, 45%, 40%, 35%, 30%, 25%, 20%, 15%, 10%, or 5% or more of the N-terminal amino acids of a Cas9 (e.g., as set forth in SEQ ID NO: 1005 or corresponding amino acid positions thereof).

In some embodiments, the circular permutant can be formed by linking a C-terminal fragment of a Cas9 to an N-terminal fragment of a Cas9, either directly or by using a linker, such as an amino acid linker. In some embodiments, the C-terminal fragment that is rearranged to the N-terminus includes or corresponds to the C-terminal 30% or less of the amino acids of a Cas9 (e.g., amino acids 1012-1368 as set forth in SEQ ID NO: 1005 or corresponding amino acid positions thereof). In some embodiments, the C-terminal fragment that is rearranged to the N-terminus, includes or corresponds to the C-terminal 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% of the amino acids of a Cas9 (e.g., as set forth in SEQ ID NO: 1005 or corresponding amino acid positions thereof). In some embodiments, the C-terminal fragment that is rearranged to the N-terminus, includes or corresponds to the C-terminal 410 residues or less of a Cas9 (e.g., as set forth in SEQ ID NO: 1005 or corresponding amino acid positions thereof). In some embodiments, the C-terminal portion that is rearranged to the N-terminus, includes or corresponds to the C-terminal 410, 400, 390, 380, 370, 360, 350, 340, 330, 320, 310,300, 290, 280, 270, 260, 250, 240, 230, 220, 210, 200, 190, 180, 170, 160, 150, 140, 130, 120, 110, 100, 90, 80, 70, 60, 50, 40, 30, 20, or 10 residues of a Cas9 (e.g., as set forth in SEQ ID NO: 1005 or corresponding amino acid positions thereof). In some embodiments, the C-terminal portion that is rearranged to the N-terminus includes or corresponds to the C-terminal 357, 341, 328, 120, or 69 residues of a Cas9 (e.g., as set forth in SEQ ID NO: 1005 or corresponding amino acid positions thereof).

In other embodiments, circular permutant Cas9 variants may be a topological rearrangement of a Cas9 primary structure based on the following method, which is based on S. pyogenes Cas9 of SEQ ID NO: 1005: (a) selecting a circular permutant (CP) site corresponding to an internal amino acid residue of the Cas9 primary structure, which dissects the original protein into two halves: an N-terminal region and a C-terminal region; (b) modifying the Cas9 protein sequence (e.g., by genetic engineering techniques) by moving the original C-terminal region (comprising the CP site amino acid) to precede the original N-terminal region, thereby forming a new N-terminus of the Cas9 protein that now begins with the CP site amino acid residue. The CP site can be located in any domain of the Cas9 protein, including, for example, the helical-II domain, the RuvCIII domain, or the CTD domain. For example, the CP site may be located (as set forth in SEQ ID NO: 1005 or corresponding amino acid positions thereof) at original amino acid residue 181, 199, 230, 270, 310, 1010, 1016, 1023, 1029, 1041, 1247, 1249, or 1282. Thus, once relocated to the N-terminus, original amino acid 181, 199, 230, 270, 310, 1010, 1016, 1023, 1029, 1041, 1247, 1249, or 1282 would become the new N-terminal amino acid. Nomenclature of these CP-Cas9 proteins may be referred to as Cas9-CP181, Cas9-CP199, Cas9-CP230, Cas9-CP270, Cas9-CP310, Cas9-CP1010, Cas9-CP1016, Cas9-CP1023, Cas9-CP1029, Cas9-CP1041, Cas9-CP1247, Cas9-CP1249, and Cas9-CP1282, respectively. This description is not meant to be limited to making CP variants from SEQ ID NO: 1005, but may be implemented to make CP variants in any Cas9 sequence, either at CP sites that correspond to these positions, or at other CP sites entirely. This description is not meant to limit the specific CP sites in any way. Virtually any CP site may be used to form a CP-Cas9 variant.

In some embodiments, a prime editor comprises a Cas9 functional variant that is of smaller molecular weight than a wild-type SpCas9 protein. In some embodiments, a smaller-sized Cas9 functional variant may facilitate delivery to cells, e.g., by an expression vector, nanoparticle, or other means of delivery. In certain embodiments, a smaller-sized Cas9 functional variant is a Class 2 Type II Cas protein. In certain embodiments, a smaller-sized Cas9 functional variant is a Class 2 Type V Cas protein. In certain embodiments, a smaller-sized Cas9 functional variant is a Class 2 Type VI Cas protein.

In some embodiments, a prime editor comprises a SpCas9 that is 1368 amino acids in length and has a predicted molecular weight of 158 kilodaltons. In some embodiments, a prime editor comprises a Cas9 functional variant or functional fragment that is less than 1300 amino acids, less than 1290 amino acids, than less than 1280 amino acids, less than 1270 amino acids, less than 1260 amino acid, less than 1250 amino acids, less than 1240 amino acids, less than 1230 amino acids, less than 1220 amino acids, less than 1210 amino acids, less than 1200 amino acids, less than 1190 amino acids, less than 1180 amino acids, less than 1170 amino acids, less than 1160 amino acids, less than 1150 amino acids, less than 1140 amino acids, less than 1130 amino acids, less than 1120 amino acids, less than 1110 amino acids, less than 1100 amino acids, less than 1050 amino acids, less than 1000 amino acids, less than 950 amino acids, less than 900 amino acids, less than 850 amino acids, less than 800 amino acids, less than 750 amino acids, less than 700 amino acids, less than 650 amino acids, less than 600 amino acids, less than 550 amino acids, or less than 500 amino acids, but at least larger than about 400 amino acids and retaining the one or more functions, e.g., DNA binding function, of the Cas9 protein.

In some embodiments, the Cas protein may include any CRISPR associated protein, including but not limited to, Cas12a, Cas12b1, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof, and preferably comprising a nickase mutation (e.g., a mutation corresponding to the D10A mutation of the wild-type SpCas9 polypeptide of SEQ ID NO: 1005). In various other embodiments, the polypeptide domain having DNA binding activity can be any of the following proteins: a Cas9, a Cas12a (Cpf1), a Cas12e (CasX), a Cas12d (CasY), a Cas12b1 (C2c1), a Cas13a (C2c2), a Cas12c (C2c3), a GeoCas9, a CjCas9, a Cas12g, a Cas12h, a Cas12i, a Cas13b, a Cas13c, a Cas13d, a Cas14, a Csn2, an xCas9, an SpCas9-NG, a circularly permuted Cas9, or an Argonaute (Ago) domain, or a functional variant or fragment thereof. Exemplary Cas proteins and nomenclature are shown in Table 2 below.

TABLE 2
Exemplary Cas proteins and nomenclature

| | |
|---|---|
| Legacy nomenclature | Current nomenclature |

type II CRISPR-Cas enzymes

| | |
|---|---|
| Cas9 | same |

type V CRISPR-Cas enzymes

| | |
|---|---|
| Cpf1 | Cas12a |
| CasX | Cas12e |
| C2c1 | Cas12b1 |
| Cas12b2 | same |
| C2c3 | Cas12c |
| CasY | Cas12d |
| C2c4 | same |
| C2c8 | same |
| C2c5 | same |
| C2c10 | same |
| C2c9 | same |

type VI CRISPR-Cas enzymes

| | |
|---|---|
| C2c2 | Cas13a |
| Cas13d | same |
| C2c7 | Cas13c |
| C2c6 | Cas13b |

In some embodiments, prime editors described herein may also comprise Cas proteins other than Cas9. For example, in some embodiments, a prime editor as described herein may comprise a Cas12a (Cpf1) polypeptide or functional variants thereof. In some embodiments, the Cas12a polypeptide comprises a mutation that reduces or abolishes the endonuclease domain of the Cas12a polypeptide. In some embodiments, the Cas12a polypeptide is a Cas12a nickase. In some embodiments, the Cas protein comprises an amino acid sequence that comprises at least about 50%, 60%, 70%, 80%, 90%, 91%, 92%, 93%, 94%, 95%, 96%, 97%, 98%, 99%, or 100% sequence identity to a naturally occurring Cas12a polypeptide.

In some embodiments, a prime editor comprises a Cas protein that is a Cas12b (C2c1) or a Cas12c (C2c3) polypeptide. In some embodiments, the Cas protein comprises an amino acid sequence that comprises at least about 50%, 60%, 70%, 80%, 90%, 91%, 92%, 93%, 94%, 95%, 96%, 97%, 98%, 99%, or 100% sequence identity to a naturally occurring Cas12b (C2c1) or Cas12c (C2c3) protein. In some embodiments, the Cas protein is a Cas12b nickase or a Cas12c nickase. In some embodiments, the Cas protein is a Cas12e, a Cas12d, a Cas13, Cas14a, Cas14b, Cas14c, Cas14d, Cas14e, Cas14f, Cas14g, Cas14h, Cas14u, or a CasΦ polypeptide. In some embodiments, the Cas protein comprises an amino acid sequence that comprises at least about 50%, 60%, 70%, 80%, 90%, 91%, 92%, 93%, 94%, 95%, 96%, 97%, 98%, 99%, or 100% sequence identity to a naturally-occurring Cas12e, Cas12d, Cas13, Cas14a, Cas14b, Cas14c, Cas14d, Cas14e, Cas14f, Cas14g, Cas14h, Cas14u, or Cas Φ protein. In some embodiments, the Cas protein is a Cas12e, Cas12d, Cas13, or Cas Φ nickase.

### Nuclear Localization Sequences

In some embodiments, a prime editor further comprises one or more nuclear localization sequence (NLS). In some embodiments, the NLS helps promote translocation of a protein into the cell nucleus. In some embodiments, a prime editor comprises a fusion protein, e.g., a fusion protein comprising a DNA binding domain and a DNA polymerase that comprises one or more NLSs. In some embodiments, one or more polypeptides of the prime editor are fused to or linked to one or more NLSs. In some embodiments, the prime editor comprises a DNA binding domain and a DNA polymerase domain that are provided in trans, wherein the DNA binding domain and/or the DNA polymerase domain is fused or linked to one or more NLSs.

In certain embodiments, a prime editor or prime editing composition comprises at least one NLS. In some embodiments, a prime editor or prime editing composition comprises at least two NLSs. In some embodiments, a prime editor or prime editing complex comprises at least three NLSs. In some embodiments, a prime editor or prime editing complex comprises more than 4, 5, 6, 7, 8, 9 or 10 NLSs. In embodiments with at least two NLSs, the NLSs can be the same NLS, or they can be different NLSs. In some embodiments, the one or more NLSs of a prime editor comprise bipartite NLSs.

NLSs can be expressed as part of a prime editor or prime editing composition. In some embodiments, a NLS can be positioned almost anywhere in a protein's amino acid sequence, and generally comprises a short sequence of three or more or four or more amino acids. The location of the NLS fusion can be at the N-terminus, the C-terminus, or positioned anywhere within a sequence of a prime editor or a component thereof (e.g., inserted between the DNA-binding domain and the DNA polymerase domain of a prime editor fusion protein, between the DNA binding domain and a linker sequence, between a DNA polymerase and a linker sequence, between two linker sequences of a prime editor fusion protein or a component thereof, in either N-terminus to C-terminus or C-terminus to N-terminus order). In some embodiments, a prime editor is a fusion protein that comprises an NLS at the N terminus. In some embodiments, a prime editor is a fusion protein that comprises an NLS at the C terminus. In some embodiments, a prime editor is a fusion protein that comprises at least one NLS at both the N terminus and the C terminus. In some embodiments, the prime editor is a fusion protein that comprises two NLSs at the N terminus and/or the C terminus.

Any NLSs that are known in the art are also contemplated herein. The NLSs may be any naturally occurring NLS, or any non-naturally occurring NLS (e.g., an NLS with one or more mutations relative to a wild-type NLS). In some embodiments, a nuclear localization signal (NLS) is predominantly basic. In some embodiments, the one or more NLSs of a prime editor are rich in lysine and arginine residues. In some embodiments, the one or more NLSs of a prime editor comprise proline residues.

Non-limiting examples of NLS sequences suitable for use with methods and compositions of the disclosure are provided in Table 3A.

In some embodiments, a NLS is a monopartite NLS. For example, in some embodiments, a NLS is a SV40 large T antigen NLS comprising the sequence SEQ ID NO: 1159. In some embodiments, a NLS is a bipartite NLS. In some embodiments, a bipartite NLS comprises two basic domains separated by a spacer sequence comprising a variable number of amino acids. In some embodiments, a NLS is a bipartite NLS. In some embodiments, a bipartite NLS consists of two basic domains separated by a spacer sequence comprising a variable number of amino acids. In some embodiments, the spacer sequence amino acid sequence comprises Xenopus nucleoplasmin NLS SEQ ID NO: 1160, wherein X is any amino acid. In some embodiments, the NLS comprises a nucleoplasmin NLS sequence SEQ ID NO: 1161. In some embodiments, a NLS is a noncanonical sequences such as M9 of the hnRNP Al protein, the influenza virus nucleoprotein NLS, and the yeast Gal4 protein NLS.

In some embodiments, a NLS comprises an amino acid sequence that is at least about 50%, 60%, 70%, 80%, 90%, 91%, 92%, 93%, 94%, 95%, 96%, 97%, 98%, 99%, or 100% identical to the amino acid sequence provided in Table 3A. In some embodiments, a NLS comprises an amino acid sequence selected from the group consisting of the amino acid sequences provided in Table 3A. In some embodiments, a prime editing composition comprises a polynucleotide that encodes a NLS that comprises an amino acid sequence that is at least about 50%, 60%, 70%, 80%, 90%, 91%, 92%, 93%, 94%, 95%, 96%, 97%, 98%, 99%, or 100% identical to the amino acid sequence provided in Table 3A. In some embodiments, a prime editing composition comprises a polynucleotide that encodes a NLS that comprises an amino acid sequence provided in Table 3A.

TABLE 3A
Exemplary nuclear localization sequences

| Description | SEQ ID NO | Sequence |
|---|---|---|
| NLS of SV40 | 1159 | PKKKRKV |
| Large T-AG | | |
| NLS | 1034 | MKRTADGSEFESPKKKRKV |
| NLS | 1153 | MDSLLMNRRKFLYQFKNVRWAKGRRETYLC |
| NLS of | 1162 | AVKRPAATKKAGQAKKKKLD |
| Nucleoplasmin | | |
| NLS of EGL-13 | 1163 | MSRRRKANPTKLSENAKKLAKEVEN |
| NLS of C-Myc | 1164 | PAAKRVKLD |
| NLS of Tus- | 1165 | KLKIKRPVK |
| protein | | |
| NLS of polyoma | 1166 | VSRKRPRP |
| large T-AG | | |
| NLS of Hepatitis | 1167 | EGAPPAKRAR |
| D virus antigen | | |
| NLS of murine | 1168 | PPQPKKKPLDGE |
| p53 | | |
| C terminal linker | 1038 | SGGSKRTADGSEFEPKKKRKV |
| and NLS of an | | |
| exemplary prime | | |
| editor fusion | | |
| protein | | |
| NLS | 1153 | MDSLLMNRRKFLYQFKNVRWAKGRRETYLC |
| NLS | 1154 | KRTADGSEFESPKKKRKV |
| NLS | 1155 | KRTADGSEFEPKKKRKV |
| NLS | 1156 | NLSKRPAAIKKAGQAKKKK |
| NLS | 1157 | RQRRNELKRSF |
| NLS | 1158 | NOSSNFGPMKGGNFGGRSSGPYGGGGQYFAKPRNQGGY |
| Xenopus | 1160 | KRXXXXXXXXXXKKKL, wherein X is any amino |
| nucleoplasmin | | acid |
| NLS | | |
| nucleoplasmin | 1161 | KRPAATKKAGQAKKKK |
| NLS sequence | | |

### Additional Prime Editor Components

A prime editor described herein may comprise additional functional domains, for example, one or more domains that modify the folding, solubility, or charge of the prime editor. In some instances, the prime editor may comprise a solubility-enhancement (SET) domain.

In some embodiments, a split intein comprises two halves of an intein protein, which may be referred to as a N-terminal half of an intein, or intein-N, and a C-terminal half of an intein, or intein-C, respectively. In some embodiments, the intein-N and the intein-C may each be fused to a protein domain (the N-terminal and the C-terminal exteins). The exteins can be any protein or polypeptides, for example, any prime editor polypeptide component. In some embodiments, the intein-N and intein-C of a split intein can associate non-covalently to form an active intein and catalyze a trans-splicing reaction. In some embodiments, the trans-splicing reaction excises the two intein sequences and links the two extein sequences with a peptide bond. As a result, the intein-N and the intein-C are spliced out, and a protein domain linked to the intein-N is fused to a protein domain linked to the intein-C essentially in same way as a contiguous intein does. In some embodiments, a split-intein is derived from a eukaryotic intein, a bacterial intein, or an archaeal intein. Preferably, the split intein so-derived will possess only the amino acid sequences essential for catalyzing trans-splicing reactions. In some embodiments, an intein-N or an intein-C further comprise one or more amino acid substitutions as compared to a wild-type intein-N or wild-type intein-C, for example, amino acid substitutions that enhances the trans-splicing activity of the split intein. In some embodiments, the intein-C comprises 4 to 7 contiguous amino acid residues, wherein at least 4 amino acids of which are from the last β-strand of the intein from which it was derived. In some embodiments, the split intein is derived from a Ssp DnaE intein, e.g., Synechocytis sp. PCC6803, or any intein or split intein known in the art, or any functional variants or fragments thereof.

In some embodiments, a prime editor comprises one or more epitope tags. Non-limiting examples of epitope tags include histidine (His) tags, V5 tags, FLAG tags, influenza hemagglutinin (HA) tags, Myc tags, VSV-G tags, thioredoxin (Trx) tags, biotin carboxylase carrier protein (BCCP) tags, myc-tags, calmodulin-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein comprises one or more His tags.

In some embodiments, a prime editor comprises one or more polypeptide domains encoded by one or more reporter genes. Examples of reporter genes include, but are not limited to, glutathione-5-transferase (GST), horseradish peroxidase (HRP), chloramphenicol acetyltransferase (CAT), beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP).

In some embodiments, a prime editor comprises one or more polypeptide domains that binds DNA molecules or binds other cellular molecules. Examples of binding proteins or domains include, but are not limited to, maltose binding protein (MBP), S-tag, Lex A DNA binding domain (DBD) fusions, GAL4 DNA binding domain fusions, and herpes simplex virus (HSV) BP16 protein fusions.

Polypeptides comprising components of a prime editor may be fused via peptide linkers, or may be provided in trans relevant to each other. For example, a reverse transcriptase may be expressed, delivered, or otherwise provided as an individual component rather than as a part of a fusion protein with the DNA binding domain. In such cases, components of the prime editor may be associated through non-peptide linkages or co-localization functions. In some embodiments, a prime editor further comprises additional components capable of interacting with, associating with, or capable of recruiting other components of the prime editor or the prime editing system. For example, a prime editor may comprise an RNA-protein recruitment polypeptide that can associate with an RNA-protein recruitment RNA aptamer. In some embodiments, an RNA-protein recruitment polypeptide can recruit, or be recruited by, a specific RNA sequence. Non limiting examples of RNA-protein recruitment polypeptide and RNA aptamer pairs include a MS2 coat protein and a MS2 RNA hairpin, a PCP polypeptide and a PP7 RNA hairpin, a Com polypeptide and a Com RNA hairpin, a Ku protein and a telomerase Ku binding RNA motif, and a Sm7 protein and a telomerase Sm7 binding RNA motif. In some embodiments, the prime editor comprises a DNA binding domain fused or linked to an RNA-protein recruitment polypeptide. In some embodiments, the prime editor comprises a DNA polymerase domain fused or linked to an RNA-protein recruitment polypeptide. In some embodiments, the DNA binding domain and the DNA polymerase domain fused to the RNA-protein recruitment polypeptide, or the DNA binding domain fused to the RNA-protein recruitment polypeptide and the DNA polymerase domain are co-localized by the corresponding RNA-protein recruitment RNA aptamer of the RNA-protein recruitment polypeptide. In some embodiments, the corresponding RNA-protein recruitment RNA aptamer is fused or linked to a portion of the PEgRNA. For example, an MS2 coat protein may be fused or linked to the DNA polymerase and a MS2 hairpin installed on the PEgRNA for co-localization of the DNA polymerase and the RNA-guided DNA binding domain (e.g., a Cas9 nickase).

In some embodiments, a prime editor comprises a polypeptide domain, an MS2 coat protein (MCP or MS2cp), that recognizes an MS2 hairpin. In some embodiments, the nucleotide sequence of the MS2 hairpin (or equivalently referred to as the “MS2 aptamer”) is a sequence as set forth in SEQ ID NO: 1169.

TABLE 3B
Exemplary MS2 hairpin and MCP sequences

| Description | Sequence | SEQ ID NO: |
|---|---|---|
| MS2 hairpin | GCCAACATGAGGATCACCCATGTCTGCAGGGCC | 1169 |
| MCP | GSASNFTQFVLVDNGGTGDVTVAPSNFANGVAEWIS | 1170 |
| | SNSRSQAYKVTCSVRQSSAQNRKYTIKVEVPKVATQ | |
| | TVGGEELPVAGWRSYLNMELTIPIFATNSDCELIVKA | |
| | MQGLLKDGNPIPSAIAANSGIY | |

In some embodiments, the amino acid sequence of the MCP is: SEQ ID NO: 1170.

In certain embodiments, components of a prime editor are directly fused to each other. In certain embodiments, components of a prime editor are associated to each other via a linker.

As used herein, a linker can be any chemical group or a molecule linking two molecules or moieties, e.g., a DNA binding domain and a polymerase domain of a prime editor. In some embodiments, a linker is an organic molecule, group, polymer, or chemical moiety. In some embodiments, the linker comprises a non-peptide moiety. The linker may be as simple as a covalent bond, or it may be a polymeric linker many atoms in length, for example, a polynucleotide sequence. In certain embodiments, the linker is a covalent bond (e.g., a carbon-carbon bond, disulfide bond, carbon-heteroatom bond, etc.).

In certain embodiments, two or more components of a prime editor are linked to each other by a peptide linker. In some embodiments, a peptide linker is 5-100 amino acids in length, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. In some embodiments, the peptide linker is 16 amino acids in length, 24 amino acids in length, 64 amino acids in length, or 96 amino acids in length. In some embodiments, a linker comprises 1-100 amino acids. Non-limiting examples of linkers are provided in Table 3C. In some embodiments, a linker comprises any one of the amino acid sequences set forth in Table 3C, or any combination thereof.

TABLE 3C
Exemplary Peptide Linker Sequences

| SEQ ID NO. | Sequence |
|---|---|
| 1171 | (GGGGS)n, wherein n is independently an integer between 1 and 30, and |
| | wherein X is any amino acid |
| 1172 | (G)n, wherein n is independently an integer between 1 and 30, and wherein X |
| | is any amino acid |
| 1173 | (EAAAK)n, wherein n is independently an integer between 1 and 30, and |
| | wherein X is any amino acid |
| 1174 | (GGS)n, wherein n is independently an integer between 1 and 30, and wherein |
| | X is any amino acid |
| 1175 | (SGGS)n, wherein n is independently an integer between 1 and 30, and wherein |
| | X is any amino acid |
| 1176 | (XP)n, wherein n is independently an integer between 1 and 30, and wherein |
| | X is any amino acid |
| 1177 | (GGS)n, wherein n is 1, 3, or 7 |
| 1178 | SGSETPGTSESATPES |
| 1179 | SGGSSGGSSGSETPGTSESATPESSGGSSGGS |
| 1180 | SGGSGGSGGS |
| 1181 | SGGS |
| 1182 | SGGSSGGSSGSETPGTSESATPESAGSYPYDVPDYAGSAAPAAKKKKLDGSGS |
| | GGSSGGS |
| 1183 | GGSGGS |
| 1184 | GGSGGSGGS |
| 1036 | SGGSSGGSSGSETPGTSESATPESSGGSSGGSS |

In certain embodiments, two or more components of a prime editor are linked to each other by a non-peptide linker. In some embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

Components of a prime editor may be connected to each other in any order. In some embodiments, the DNA binding domain and the DNA polymerase domain of a prime editor may be fused to form a fusion protein, or may be joined by a peptide or protein linker, in any order from the N terminus to the C terminus. In some embodiments, a prime editor comprises a DNA binding domain fused or linked to the C-terminal end of a DNA polymerase domain. In some embodiments, a prime editor comprises a DNA binding domain fused or linked to the N-terminal end of a DNA polymerase domain. In some embodiments, the prime editor comprises a fusion protein comprising the structure NH2-[DNA binding domain]-[DNA polymerase]-COOH; or NH2-[DNA polymerase]-[DNA binding domain]-COOH, wherein each instance of “]-[” indicates the presence of an optional linker sequence. In some embodiments, a prime editor comprises a fusion protein and a DNA polymerase domain provided in trans, wherein the fusion protein comprises the structure NH2-[DNA binding domain]-[RNA-protein recruitment polypeptide]-COOH. In some embodiments, a prime editor comprises a fusion protein and a DNA binding domain provided in trans, wherein the fusion protein comprises the structure NH2-[DNA polymerase domain]-[RNA-protein recruitment polypeptide]-COOH.

In some embodiments, a prime editor fusion protein, a polypeptide component of a prime editor, or a polynucleotide encoding the prime editor fusion protein or polypeptide component, may be split into an N-terminal half and a C-terminal half or polypeptides that encode the N-terminal half and the C terminal half, and provided to a target DNA in a cell separately. For example, in certain embodiments, a prime editor fusion protein may be split into a N-terminal and a C-terminal half for separate delivery in AAV vectors, and subsequently translated and colocalized in a target cell to reform the complete polypeptide or prime editor protein. In such cases, separate halves of a protein or a fusion protein may each comprise a split-intein to facilitate colocalization and reformation of the complete protein or fusion protein by the mechanism of intein facilitated trans-splicing. In some embodiments, a prime editor comprises a N-terminal half fused to an intein-N, and a C-terminal half fused to an intein-C, or polynucleotides or vectors (e.g., AAV vectors) encoding each thereof. When delivered and/or expressed in a target cell, the intein-N and the intein-C can be excised via protein trans-splicing, resulting in a complete prime editor fusion protein in the target cell. In some embodiments, an exemplary protein described herein may lack a methionine residue at the N-terminus.

In some embodiments, a prime editor (e.g., a prime editor fusion protein) comprises one or more individual components shown in Tables 4A or 4B. In some embodiments, a prime editor is a prime editor fusion protein comprising all of the components shown in Table 4A or 4B.

In some embodiments, a prime editor fusion protein comprises a Cas9 (H840A) nickase and a wild type M-MLV RT. In some embodiments, a prime editor fusion protein comprises a Cas9 (H840A) nickase and a M-MLV RT that comprises amino acid substitutions D200N, T330P, T306K, W313F, and L603W compared to a wild type M-MLV RT. In some embodiments, a prime editor fusion protein comprises a Cas9 (H840A) nickase and a M-MLV RT that comprises amino acid substitutions D200N, T330P, T306K, W313F, and L603W compared to a wild type M-MLV RT or a variant M-MLV RT of SEQ ID NO: 1003. The amino acid sequence of an exemplary prime editor fusion protein and its individual components is shown in Table 4A.

In some embodiments, a prime editor fusion protein comprises a Cas9 (R221K N394K H840A) nickase and a M-MLV RT that comprises amino acid substitutions D200N, T330P, T306K, W313F, and L603W compared to a wild type M-MLV RT or a variant M-MLV RT of SEQ ID NO: 1003. The amino acid sequence of an exemplary Prime editor fusion protein and its individual components in shown in Table 4B.

In some embodiments an exemplary prime editor protein may comprise an amino acid sequence as set forth in any of the SEQ ID NO: 1033 or SEQ ID NO: 1039.

In some embodiments, a prime editing composition comprises a fusion protein comprising a DNA binding domain (e.g., Cas9(H840A)) and a reverse transcriptase (e.g., a variant M-MLV RT) having the following structure: [NLS]-[Cas9(H840A)]-[linker]-[M-MLV_RT(D200N)(T330P)(L603W)(T306K)(W313F)], and one or more desired PEgRNAs. In some embodiments, the prime editing composition comprises a prime editor fusion protein that has the amino acid sequence of SEQ ID NO:1033. Sequence of an exemplary prime editor fusion protein comprising a DNA binding domain (e.g., Cas9(H840A)) and a reverse transcriptase (e.g., a variant MMLV RT) having the following structure: [NLS]-[Cas9(H840A)]-[linker]-[MMLV_RT(D200N)(T330P)(L603W)(T306K)(W313F)] and its components are shown in Table 4A.

In various embodiments, a prime editor or its components comprise an amino acid sequence that is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to a sequence in Table 4A.

In some embodiments, a prime editing complex comprises a fusion protein comprising a DNA binding domain (e.g., Cas9((R221K N394K H840A)) and a reverse transcriptase (e.g., a variant MMLV RT) having the following structure: [NLS]-[Cas9((R221K N394K H840A)]-[linker]-[MMLV_RT(D200N)(T330P)(L603W)(T306K)(W313F)], and a desired PERNA. In some embodiments, the prime editing complex comprises a prime editor fusion protein that has the amino acid sequence of SEQ ID NO: 1039. Sequence of an exemplary prime editor fusion protein comprising a DNA binding domain (e.g., Cas9(H840A)) and a reverse transcriptase (e.g., a variant MMLV RT) having the following structure: [NLS]-[Cas9(R221K N394K H840A)]-[linker]-[MMLV_RT(D200N)(T330P)(L603W)(T306K)(W313F)] and its components are shown in Table 4B.

In some embodiments, a prime editor or its components comprises an amino acid sequence that is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to a sequence in Table 4B.

TABLE 4A
Amino acid sequence of an Exemplary Prime Editor and its individual components

| Description | Sequence |
|---|---|
| Exemplary | MKRTADGSEFESPKKKRKVDKKYSIGLDIGTNSVGWAVITDEYKVPSKKF |
| PRIME | KVLGNTDRHSIKKNLIGALLFDSGETAEATRLKRTARRRYTRRKNRICYL |
| EDITOR | QEIFSNEMAKVDDSFFHRLEESFLVEEDKKHERHPIFGNIVDEVAYHEKY |
| FUSION | PTIYHLRKKLVDSTDKADLRLIYLALAHMIKFRGHFLIEGDLNPDNSDVD |
| PROTEIN | KLFIQLVQTYNQLFEENPINASGVDAKAILSARLSKSRRLENLIAQLPGEK |
| CAS9(H840A)- | KNGLFGNLIALSLGLTPNFKSNFDLAEDAKLQLSKDTYDDDLDNLLAQIG |
| MMLV_RT | DQYADLFLAAKNLSDAILLSDILRVNTEITKAPLSASMIKRYDEHHQDLTL |
| D200N T330P | LKALVRQQLPEKYKEIFFDQSKNGYAGYIDGGASQEEFYKFIKPILEKMD |
| L603W | GTEELLVKLNREDLLRKQRTFDNGSIPHQIHLGELHAILRRQEDFYPFLK |
| T306K | DNREKIEKILTFRIPYYVGPLARGNSRFAWMTRKSEETITPWNFEEVVDK |
| W313F | GASAQSFIERMTNFDKNLPNEKVLPKHSLLYEYFTVYNELTKVKYVTEG |
| | MRKPAFLSGEQKKAIVDLLFKTNRKVTVKQLKEDYFKKIECFDSVEISGV |
| | EDRFNASLGTYHDLLKIIKDKDFLDNEENEDILEDIVLTLTLFEDREMIEE |
| | RLKTYAHLFDDKVMKQLKRRRYTGWGRLSRKLINGIRDKQSGKTILDF |
| | LKSDGFANRNFMQLIHDDSLTFKEDIQKAQVSGQGDSLHEHIANLAGSPA |
| | IKKGILQTVKVVDELVKVMGRHKPENIVIEMARENQTTQKGQKNSRER |
| | MKRIEEGIKELGSQILKEHPVENTQLQNEKLYLYYLQNGRDMYVDQELD |
| | INRLSDYDVDAIVPQSFLKDDSIDNKVLTRSDKNRGKSDNVPSEEVVKKM |
| | KNYWRQLLNAKLITQRKFDNLTKAERGGLSELDKAGFIKRQLVETRQIT |
| | KHVAQILDSRMNTKYDENDKLIREVKVITLKSKLVSDFRKDFQFYKVREI |
| | NNYHHAHDAYLNAVVGTALIKKYPKLESEFVYGDYKVYDVRKMIAKSE |
| | QEIGKATAKYFFYSNIMNFFKTEITLANGEIRKRPLIETNGETGEIVWDKG |
| | RDFATVRKVLSMPQVNIVKKTEVQTGGFSKESILPKRNSDKLIARKKDW |
| | DPKKYGGFDSPTVAYSVLVVAKVEKGKSKKLKSVKELLGITIMERSSFEK |
| | NPIDFLEAKGYKEVKKDLIIKLPKYSLFELENGRKRMLASAGELQKGNEL |
| | ALPSKYVNFLYLASHYEKLKGSPEDNEQKQLFVEQHKHYLDEIIEQISEFS |
| | KRVILADANLDKVLSAYNKHRDKPIREQAENIIHLFTLTNLGAPAAFKYF |
| | DTTIDRKRYTSTKEVLDATLIHQSITGLYETRIDLSQLGGDSGGSSGGSSG |
| | SETPGTSESATPESSGGSSGGSSTLNIEDEYRLHETSKEPDVSLGSTWLSDFPQA |
| | WAETGGMGLAVRQAPLIIPLKATSTPVSIKQYPMSQEARLGIKPHIQRLLDQGILVP |
| | CQSPWNTPLLPVKKPGTNDYRPVQDLREVNKRVEDIHPTVPNPYNLLSGLPPSHQ |
| | WYTVLDLKDAFFCLRLHPTSQPLFAFEWRDPEMGISGQLTWTRLPQGFKNSPTLF |
| | NEALHRDLADFRIQHPDLILLQYVDDLLLAATSELDCQQGTRALLQTLGNLGYRAS |
| | AKKAQICQKQVKYLGYLLKEGQRWLTEARKETVMGQPTPKTPRQLREFLGKAGF |
| | CRLFIPGFAEMAAPLYPLTKPGTLFNWGPDQQKAYQEIKQALLTAPALGLPDLTK |
| | PFELFVDEKQGYAKGVLTQKLGPWRRPVAYLSKKLDPVAAGWPPCLRMVAAIAV |
| | LTKDAGKLTMGQPLVILAPHAVEALVKQPPDRWLSNARMTHYQALLLDTDRVQF |
| | GPVVALNPATLLPLPEEGLQHNCLDILAEAHGTRPDLTDQPLPDADHTWYTDGSS |
| | LLQEGQRKAGAAVTTETEVIWAKALPAGTSAQRAELIALTQALKMAEGKKLNVYT |
| | DSRYAFATAHIHGEIYRRRGWLTSEGKEIKNKDEILALLKALFLPKRLSIIHCPGHQ |
| | KGHSAEARGNRMADQAARKAAITETPDTSTLLIENSSPSGGSKRTADGSEFEPKK |
| | KRKV (SEQ ID NO: 1033) |
| | KEY: |
| | NUCLEAR LOCALIZATION SEQUENCE (NLS) |
| | CAS9(H840A) |
| | 33-AMINO ACID LINKER |
| | M-MLV REVERSE TRANSCRIPTASE |
| N-Terminal | MKRTADGSEFESPKKKRKV (SEQ ID NO: 1034) |
| NLS | |
| Cas9 domain | DKKYSIGLDIGTNSVGWAVITDEYKVPSKKFKVLGNTDRHSIKKNLIGALLFD |
| (H840A | SGETAEATRLKRTARRRYTRRKNRICYLQEIFSNEMAKVDDSFFHRLEESFLV |
| nickase, | EEDKKHERHPIFGNIVDEVAYHEKYPTIYHLRKKLVDSTDKADLRLIYLALAH |
| wherein the | MIKFRGHFLIEGDLNPDNSDVDKLFIQLVQTYNQLFEENPINASGVDAKAILS |
| amino acid | ARLSKSRRLENLIAQLPGEKKNGLFGNLIALSLGLTPNFKSNFDLAEDAKLQL |
| positioning is | SKDTYDDDLDNLLAQIGDQYADLFLAAKNLSDAILLSDILRVNTEITKAPLSA |
| in reference to | SMIKRYDEHHQDLTLLKALVRQQLPEKYKEIFFDQSKNGYAGYIDGGASQEE |
| full length | FYKFIKPILEKMDGTEELLVKLNREDLLRKQRTFDNGSIPHQIHLGELHAILRR |
| wild type | QEDFYPFLKDNREKIEKILTFRIPYYVGPLARGNSRFAWMTRKSEETITPWNFE |
| spCas9 (SEQ | EVVDKGASAQSFIERMTNFDKNLPNEKVLPKHSLLYEYFTVYNELTKVKYVT |
| ID NO: | EGMRKPAFLSGEQKKAIVDLLFKTNRKVTVKQLKEDYFKKIECFDSVEISGVE |
| 1005)) | DRFNASLGTYHDLLKIIKDKDFLDNEENEDILEDIVLTLTLFEDREMIEERLKT |
| | YAHLFDDKVMKQLKRRRYTGWGRLSRKLINGIRDKQSGKTILDFLKSDGFA |
| | NRNFMQLIHDDSLTFKEDIQKAQVSGQGDSLHEHIANLAGSPAIKKGILQTVK |
| | VVDELVKVMGRHKPENIVIEMARENQTTQKGQKNSRERMKRIEEGIKELGSQ |
| | ILKEHPVENTQLQNEKLYLYYLQNGRDMYVDQELDINRLSDYDVDAIVPQSF |
| | LKDDSIDNKVLTRSDKNRGKSDNVPSEEVVKKMKNYWRQLLNAKLITQRKF |
| | DNLTKAERGGLSELDKAGFIKRQLVETRQITKHVAQILDSRMNTKYDENDKL |
| | IREVKVITLKSKLVSDFRKDFQFYKVREINNYHHAHDAYLNAVVGTALIKKY |
| | PKLESEFVYGDYKVYDVRKMIAKSEQEIGKATAKYFFYSNIMNFFKTEITLAN |
| | GEIRKRPLIETNGETGEIVWDKGRDFATVRKVLSMPQVNIVKKTEVQTGGFS |
| | KESILPKRNSDKLIARKKDWDPKKYGGFDSPTVAYSVLVVAKVEKGKSKKL |
| | KSVKELLGITIMERSSFEKNPIDFLEAKGYKEVKKDLIIKLPKYSLFELENGRK |
| | RMLASAGELQKGNELALPSKYVNFLYLASHYEKLKGSPEDNEQKQLFVEQH |
| | KHYLDEIIEQISEFSKRVILADANLDKVLSAYNKHRDKPIREQAENIIHLFTLTN |
| | LGAPAAFKYFDTTIDRKRYTSTKEVLDATLIHQSITGLYETRIDLSQLGGD |
| | (SEQ ID NO: 1007) |
| Linker | SGGSSGGSSGSETPGTSESATPESSGGSSGGSS (SEQ ID NO: 1036) |
| between | |
| Cas9 domain | |
| and RT | |
| domain (33 | |
| amino acids) | |
| MMLV_RT | TLNIEDEYRLHETSKEPDVSLGSTWLSDFPQAWAETGGMGLAVRQAPLIIPLK |
| D200N | ATSTPVSIKQYPMSQEARLGIKPHIQRLLDQGILVPCQSPWNTPLLPVKKPGTN |
| T330P | DYRPVQDLREVNKRVEDIHPTVPNPYNLLSGLPPSHQWYTVLDLKDAFFCLR |
| L603W | LHPTSQPLFAFEWRDPEMGISGQLTWTRLPQGFKNSPTLFNEALHRDLADFRI |
| T306K | QHPDLILLQYVDDLLLAATSELDCQQGTRALLQTLGNLGYRASAKKAQICQK |
| W313F | QVKYLGYLLKEGQRWLTEARKETVMGQPTPKTPRQLREFLGKAGFCRLFIPG |
| | FAEMAAPLYPLTKPGTLFNWGPDQQKAYQEIKQALLTAPALGLPDLTKPFEL |
| | FVDEKQGYAKGVLTQKLGPWRRPVAYLSKKLDPVAAGWPPCLRMVAAIAV |
| | LTKDAGKLTMGQPLVILAPHAVEALVKQPPDRWLSNARMTHYQALLLDTDR |
| | VQFGPVVALNPATLLPLPEEGLQHNCLDILAEAHGTRPDLTDQPLPDADHTW |
| | YTDGSSLLQEGQRKAGAAVTTETEVIWAKALPAGTSAQRAELIALTQALKM |
| | AEGKKLNVYTDSRYAFATAHIHGEIYRRRGWLTSEGKEIKNKDEILALLKAL |
| | FLPKRLSIIHCPGHQKGHSAEARGNRMADQAARKAAITETPDTSTLLIENSSP |
| | (SEQ ID NO: 1003) |
| C- terminal | SGGSKRTADGSEFEPKKKRKV (SEQ ID NO: 1038) |
| NLS | |

TABLE 4B
Amino acid sequence of an Exemplary Prime Editor and its individual components

| DESCRIPTION | SEQUENCE |
|---|---|
| Exemplary | MKRTADGSEFESPKKKRKVDKKYSIGLDIGTNSVGWAVITDEYKVPSKKF |
| prime editor | KVLGNTDRHSIKKNLIGALLFDSGETAEATRLKRTARRRYTRRKNRICYL |
| [NLS]- | QEIFSNEMAKVDDSFFHRLEESFLVEEDKKHERHPIFGNIVDEVAYHEKY |
| [Cas9((R220K) | PTIYHLRKKLVDSTDKADLRLIYLALAHMIKFRGHFLIEGDLNPDNSDVD |
| (R393K) | KLFIQLVQTYNQLFEENPINASGVDAKAILSARLSKSRKLENLIAQLPGEK |
| (H839A)]- | KNGLFGNLIALSLGLTPNFKSNFDLAEDAKLQLSKDTYDDDLDNLLAQIG |
| [linker]- | DQYADLFLAAKNLSDAILLSDILRVNTEITKAPLSASMIKRYDEHHQDLTL |
| [MMLV_RT | LKALVRQQLPEKYKEIFFDQSKNGYAGYIDGGASQEEFYKFIKPILEKMD |
| (D200N)(T330P) | GTEELLVKLKREDLLRKQRTFDNGSIPHQIHLGELHAILRRQEDFYPFLK |
| (L603W)(T306K) | DNREKIEKILTFRIPYYVGPLARGNSRFAWMTRKSEETITPWNFEEVVDK |
| (W313F)]- | GASAQSFIERMTNFDKNLPNEKVLPKHSLLYEYFTVYNELTKVKYVTEG |
| [NLS] | MRKPAFLSGEQKKAIVDLLFKTNRKVTVKQLKEDYFKKIECFDSVEISGV |
| | EDRFNASLGTYHDLLKIIKDKDFLDNEENEDILEDIVLTLTLFEDREMIEE |
| | RLKTYAHLFDDKVMKQLKRRRYTGWGRLSRKLINGIRDKQSGKTILDFL |
| | KSDGFANRNFMQLIHDDSLTFKEDIQKAQVSGQGDSLHEHIANLAGSPAI |
| | KKGILQTVKVVDELVKVMGRHKPENIVIEMARENQTTQKGQKNSRERM |
| | KRIEEGIKELGSQILKEHPVENTQLQNEKLYLYYLQNGRDMYVDQELDI |
| | NRLSDYDVDAIVPQSFLKDDSIDNKVLTRSDKNRGKSDNVPSEEVVKKMK |
| | NYWRQLLNAKLITQRKFDNLTKAERGGLSELDKAGFIKRQLVETRQITK |
| | HVAQILDSRMNTKYDENDKLIREVKVITLKSKLVSDFRKDFQFYKVREIN |
| | NYHHAHDAYLNAVVGTALIKKYPKLESEFVYGDYKVYDVRKMIAKSEQ |
| | EIGKATAKYFFYSNIMNFFKTEITLANGEIRKRPLIETNGETGEIVWDKGR |
| | DFATVRKVLSMPQVNIVKKTEVQTGGFSKESILPKRNSDKLIARKKDWD |
| | PKKYGGFDSPTVAYSVLVVAKVEKGKSKKLKSVKELLGITIMERSSFEKN |
| | PIDFLEAKGYKEVKKDLIIKLPKYSLFELENGRKRMLASAGELQKGNELA |
| | LPSKYVNFLYLASHYEKLKGSPEDNEQKQLFVEQHKHYLDEIIEQISEFSK |
| | RVILADANLDKVLSAYNKHRDKPIREQAENIIHLFTLTNLGAPAAFKYFD |
| | TTIDRKRYTSTKEVLDATLIHQSITGLYETRIDLSQLGGDSGGSSGGSKRT |
| | ADGSEFESPKKKRKVSGGSSGGSTLNIEDEYRLHETSKEPDVSLGSTWLSDFPQ |
| | AWAETGGMGLAVRQAPLIIPLKATSTPVSIKQYPMSQEARLGIKPHIQRLLDQGILV |
| | PCQSPWNTPLLPVKKPGTNDYRPVQDLREVNKRVEDIHPTVPNPYNLLSGLPPSH |
| | QWYTVLDLKDAFFCLRLHPTSQPLFAFEWRDPEMGISGQLTWTRLPQGFKNSPTL |
| | FNEALHRDLADFRIQHPDLILLQYVDDLLLAATSELDCQQGTRALLQTLGNLGYRA |
| | SAKKAQICQKQVKYLGYLLKEGQRWLTEARKETVMGQPTPKTPRQLREFLGKAG |
| | FCRLFIPGFAEMAAPLYPLTKPGTLFNWGPDQQKAYQEIKQALLTAPALGLPDLT |
| | KPFELFVDEKQGYAKGVLTQKLGPWRRPVAYLSKKLDPVAAGWPPCLRMVAAIA |
| | VLTKDAGKLTMGQPLVILAPHAVEALVKQPPDRWLSNARMTHYQALLLDTDRVQ |
| | FGPVVALNPATLLPLPEEGLQHNCLDILAEAHGTRPDLTDQPLPDADHTWYTDGS |
| | SLLQEGQRKAGAAVTTETEVIWAKALPAGTSAQRAELIALTQALKMAEGKKLNVYT |
| | DSRYAFATAHIHGEIYRRRGWLTSEGKEIKNKDEILALLKALFLPKRLSIIHCPGHQ |
| | KGHSAEARGNRMADQAARKAAITETPDTSTLLIENSSPSGGSKRTADGSEFESPKK |
| | KRKVGSGPAAKRVKLD (SEQ ID NO: 1039) |
| | KEY: |
| | N-terminal bipartiteSV40NLS |
| | CAS9(R221K N394K H840A) |
| | SGGSx2-met-bpSV40NLS-SGGSx2 LINKER |
| | M-MLV D200N T306K W313F T330P L603W REVERSE TRANSCRIPTASE |
| | C-terminal linker-NLS1 |
| | C-terminal linker-NLS2 |
| -N-terminal | MKRTADGSEFESPKKKRKV (SEQ ID NO: 1034) |
| bpSV40NLS | |
| - Cas9 | DKKYSIGLDIGTNSVGWAVITDEYKVPSKKFKVLGNTDRHSIKKNLIGALLFD |
| (R221K | SGETAEATRLKRTARRRYTRRKNRICYLQEIFSNEMAKVDDSFFHRLEESFLV |
| N394K | EEDKKHERHPIFGNIVDEVAYHEKYPTIYHLRKKLVDSTDKADLRLIYLALAH |
| H840A) | MIKFRGHFLIEGDLNPDNSDVDKLFIQLVQTYNQLFEENPINASGVDAKAILSA |
| | RLSKSRKLENLIAQLPGEKKNGLFGNLIALSLGLTPNFKSNFDLAEDAKLQLS |
| | KDTYDDDLDNLLAQIGDQYADLFLAAKNLSDAILLSDILRVNTEITKAPLSAS |
| | MIKRYDEHHQDLTLLKALVRQQLPEKYKEIFFDQSKNGYAGYIDGGASQEEF |
| | YKFIKPILEKMDGTEELLVKLKREDLLRKQRTFDNGSIPHQIHLGELHAILRRQ |
| | EDFYPFLKDNREKIEKILTFRIPYYVGPLARGNSRFAWMTRKSEETITPWNFEE |
| | VVDKGASAQSFIERMTNFDKNLPNEKVLPKHSLLYEYFTVYNELTKVKYVTE |
| | GMRKPAFLSGEQKKAIVDLLFKTNRKVTVKQLKEDYFKKIECFDSVEISGVED |
| | RFNASLGTYHDLLKIIKDKDFLDNEENEDILEDIVLTLTLFEDREMIEERLKTY |
| | AHLFDDKVMKQLKRRRYTGWGRLSRKLINGIRDKQSGKTILDFLKSDGFANR |
| | NFMQLIHDDSLTFKEDIQKAQVSGQGDSLHEHIANLAGSPAIKKGILQTVKVV |
| | DELVKVMGRHKPENIVIEMARENQTTQKGQKNSRERMKRIEEGIKELGSQILK |
| | EHPVENTQLQNEKLYLYYLQNGRDMYVDQELDINRLSDYDVDAIVPQSFLK |
| | DDSIDNKVLTRSDKNRGKSDNVPSEEVVKKMKNYWRQLLNAKLITQRKFDN |
| | LTKAERGGLSELDKAGFIKRQLVETRQITKHVAQILDSRMNTKYDENDKLIRE |
| | VKVITLKSKLVSDFRKDFQFYKVREINNYHHAHDAYLNAVVGTALIKKYPKL |
| | ESEFVYGDYKVYDVRKMIAKSEQEIGKATAKYFFYSNIMNFFKTEITLANGEI |
| | RKRPLIETNGETGEIVWDKGRDFATVRKVLSMPQVNIVKKTEVQTGGFSKESI |
| | LPKRNSDKLIARKKDWDPKKYGGFDSPTVAYSVLVVAKVEKGKSKKLKSVK |
| | ELLGITIMERSSFEKNPIDFLEAKGYKEVKKDLIIKLPKYSLFELENGRKRMLA |
| | SAGELQKGNELALPSKYVNFLYLASHYEKLKGSPEDNEQKQLFVEQHKHYL |
| | DEIIEQISEFSKRVILADANLDKVLSAYNKHRDKPIREQAENIIHLFTLTNLGAP |
| | AAFKYFDTTIDRKRYTSTKEVLDATLIHQSITGLYETRIDLSQLGGD (SEQ ID |
| | NO: 1008) |
| -SGGSx2- | SGGSSGGSKRTADGSEFESPKKKRKVSGGSSGGS (SEQ ID NO: 1042) |
| bpSV40NLS- | |
| SGGSx2 | |
| linker | |
| -MMLV_RT | TLNIEDEYRLHETSKEPDVSLGSTWLSDFPQAWAETGGMGLAVRQAPLIIPLK |
| D200N T330P | ATSTPVSIKQYPMSQEARLGIKPHIQRLLDQGILVPCQSPWNTPLLPVKKPGTN |
| L603W | DYRPVQDLREVNKRVEDIHPTVPNPYNLLSGLPPSHQWYTVLDLKDAFFCLR |
| T306K | LHPTSQPLFAFEWRDPEMGISGQLTWTRLPQGFKNSPTLFNEALHRDLADFRI |
| W313F | QHPDLILLQYVDDLLLAATSELDCQQGTRALLQTLGNLGYRASAKKAQICQK |
| | QVKYLGYLLKEGQRWLTEARKETVMGQPTPKTPRQLREFLGKAGFCRLFIPG |
| | FAEMAAPLYPLTKPGTLFNWGPDQQKAYQEIKQALLTAPALGLPDLTKPFEL |
| | FVDEKQGYAKGVLTQKLGPWRRPVAYLSKKLDPVAAGWPPCLRMVAAIAV |
| | LTKDAGKLTMGQPLVILAPHAVEALVKQPPDRWLSNARMTHYQALLLDTDR |
| | VQFGPVVALNPATLLPLPEEGLQHNCLDILAEAHGTRPDLTDQPLPDADHTW |
| | YTDGSSLLQEGQRKAGAAVTTETEVIWAKALPAGTSAQRAELIALTQALKMA |
| | EGKKLNVYTDSRYAFATAHIHGEIYRRRGWLTSEGKEIKNKDEILALLKALFL |
| | PKRLSIIHCPGHQKGHSAEARGNRMADQAARKAAITETPDTSTLLIENSSP |
| | (SEQ ID NO: 1003) |
| C-terminal | SGGSKRTADGSEFESPKKKRKV (SEQ ID NO: 1044) |
| linker-NLS | |
| C-terminal | GSGPAAKRVKLD (SEQ ID NO: 1045) |
| linker-NLS2 | |

### PEgRNAs for Editing of TRAC Gene

Provided herein are systems, compositions, and methods, including prime editing guide RNAs (PEgRNAs), pairs of PEgRNAs, and prime editing compositions for editing of the human TRAC gene.

Exemplary nucleic acid sequence of the TRAC gene is available, for example, at UCSC human genome database, Gene ENSG00000277734.8, which sequence is incorporated herein by reference in its entirety.

Exemplary wild-type TRAC protein sequence NCBI Accession No. P01848.2: IQNPDPAVYQLRDSKSSDKSVCLFTDFDSQTNVSQSKDSDVYITDKTVLDMRSMDFKSNSAV AWSNKSDFACANAFNNSIIPEDTFFPSPESSCDVKLVEKSFETDTNLNFQNLSVIGFRILLLKVA GFNLLMTLRLWSS (SEQ ID NO: 1185)

Exemplary wild-type TRAC mRNA/cDNA sequence:

(SEQ ID NO: 1186)
TTTTGAAACCCTTCAAAGGCAGAGACTTGTCCAGCCTAACCTGCCTGCT
GCTCCTAGCTCCTGAGGCTCAGGGCCCTTGGCTTCTGTCCGCTCTGCTC
AGGGCCCTCCAGCGTGGCCACTGCTCAGCCATGCTCCTGCTGCTCGTCC
CAGTGCTCGAGGTGATTTTTACCCTGGGAGGAACCAGAGCCCAGTCGGT
GACCCAGCTTGGCAGCCACGTCTCTGTCTCTGAAGGAGCCCTGGTTCTG
CTGAGGTGCAACTACTCATCGTCTGTTCCACCATATCTCTTCTGGTATG
TGCAATACCCCAACCAAGGACTCCAGCTTCTCCTGAAGTACACATCAGC
GGCCACCCTGGTTAAAGGCATCAACGGTTTTGAGGCTGAATTTAAGAAG
AGTGAAACCTCCTTCCACCTGACGAAACCCTCAGCCCATATGAGCGACG
CGGCTGAGTACTTCTGTGCTGTGAGTGATCTCGAACCGAACAGCAGTGC
TTCCAAGATAATCTTTGGATCAGGGACCAGACTCAGCATCCGGCCAAAT
ATCCAGAACCCTGACCCTGCCGTGTACCAGCTGAGAGACTCTAAATCCA
GTGACAAGTCTGTCTGCCTATTCACCGATTTTGATTCTCAAACAAATGT
GTCACAAAGTAAGGATTCTGATGTGTATATCACAGACAAAACTGTGCTA
GACATGAGGTCTATGGACTTCAAGAGCAACAGTGCTGTGGCCTGGAGCA
ACAAATCTGACTTTGCATGTGCAAACGCCTTCAACAACAGCATTATTCC
AGAAGACACCTTCTTCCCCAGCCCAGAAAGTTCCTGTGATGTCAAGCTG
GTCGAGAAAAGCTTTGAAACAGATACGAACCTAAACTTTCAAAACCTGT
CAGTGATTGGGTTCCGAATCCTCCTCCTGAAAGTGGCCGGGTTTAATCT
GCTCATGACGCTGCGGCTGTGGTCCAGCTGAGATCTGCAAGATTGTAAG
ACAGCCTGTGCTCCCTCGCTCCTTCCTCTGCATTGCCCCTCTTCTCCCT
CTCCAAACAGAGGGAACTCTCCTACCCCCAAGGAGGTGAAAGCTGCTAC
CACCTCTGTGCCCCCCCGGTAATGCCACCAACTGGATCCTACCCGAATT
TATGATTAAGATTGCTGAAGAGCTGCCAAACACTGCTGCCACCCCCTCT
GTTCCCTTATTGCTGCTTGTCACTGCCTGACATTCACGGCAGAGGCAAG
GCTGCTGCAGCCTCCCCTGGCTGTGCACATTCCCTCCTGCTCCCCAGAG
ACTGCCTCCGCCATCCCACAGATGATGGATCTTCAGTGGGTTCTCTTGG
GCTCTAGGTCCTGGAGAATGTTGTGAGGGGTTTATTTTTTTTTAATAGT
GTTCATAAAGAAATACATAGTATTCTTCTTCTCAAGACGTGGGGGGAAA
TTATCTCATTATCGAGGCCCTGCTATGCTGTGTGTCTGGGCGTGTTGTA
TGTCCTGCTGCCGATGCCTTCATTAAAATGATTTGGAA

The term “prime editing guide RNA”, or “PEgRNA”, refers to a guide polynucleotide that comprises one or more intended nucleotide edits for incorporation into the target double-stranded DNA. In some embodiments, the PEgRNA associates with and directs a prime editor to incorporate the one or more intended nucleotide edits into the target gene via prime editing.

In some embodiments, a PEgRNA comprises a spacer that is complementary or substantially complementary to a search target sequence on a target strand of the target gene. In some embodiments, the PEgRNA comprises a gRNA core that associates with a DNA binding domain, e.g., a CRISPR-Cas protein domain, of a prime editor. In some embodiments, the PEgRNA comprises an editing template. In some embodiments, the PERNA comprises a primer binding site (PBS). In some embodiments, a PEgRNA comprises an extension arm that comprises an editing template and a primer binding site (PBS). In some embodiments, the editing template of a PEgRNA encodes one or more nucleotide edits compared to the endogenous TRAC gene sequence, e.g., the editing target sequence. In some embodiments, the one or more nucleotide edits comprise insertion, deletion, and/or substitution of one or more nucleotides. In some embodiments, the one or more nucleotide edits comprise insertion of an exogenous sequence, e.g., a recombinase recognition sequence (RSS). In some embodiments, the one or more nucleotide edits comprise insertion of one or more RSSs.

Dual prime editing involves two different PEgRNAs each complexed with a prime editor. In some embodiments, the prime editor is the same for each of the PEgRNA-prime editor complexes. In some embodiments, the prime editor is different for each of the PEgRNA-prime editor complexes. In some embodiments, each of the two PEgRNAs comprises a region of complementarity to a distinct search target sequence of the double-stranded target DNA, wherein the two distinct search target sequences are on the two complementary strands of the double-stranded target DNA. In some embodiments, the two PEgRNAs each can direct a prime editor to initiate the prime editing process on the two complementary strands of the double-stranded target DNA. In some embodiments, each of the two PEgRNAs comprises a spacer complementary to a separate search target sequence. In some embodiments, each of the two PEgRNAs anneals with a separate search target sequence through its spacer.

In some embodiments, a first PEgRNA comprises a first spacer complementary to a first search target sequence on a first strand of a double-stranded target DNA, e.g., a double-stranded target gene. In the context of the first PEgRNA, the first strand of the double-stranded target DNA may be referred to as a first target strand, and the complementary strand referred to as the first PAM strand. In some embodiments, a first PEgRNA comprises a first gRNA core. In some embodiments, a first PEgRNA comprises a first editing template. In certain embodiments, a first PEgRNA comprises a first primer binding site (PBS) that is complementary to a free 3′ end formed at the first nick site.

In some embodiments, a second PEgRNA comprises a second spacer complementary to a second search target sequence on a second strand of a double-stranded target DNA, e.g., a double-stranded target gene. In the context of the second PEgRNA, the second strand of the double-stranded target DNA may be referred to as a second target strand, and the complementary strand referred to as the second PAM strand. In some embodiments, a second PEgRNA comprises a second gRNA core. In some embodiments, a second PEgRNA comprises a second editing template. In some embodiments, a second PEgRNA comprises a second primer binding site (PBS) that is complementary to a free 3′ end formed at the second nick site.

The specific sequences of the first and the second spacer can determine positions of the two nick sites made by prime editor(s) complexed with the first and second PEgRNA. In some embodiments, dual prime editing results in replacement of the sequence between the first and second nick (i.e. the IND) by single stranded sequences encoded by the RTTs (the replacement duplex, RD) in the target gene, e.g., the TRAC gene. In some embodiments, incorporation of the one or more intended nucleotide edits encoded by the RTTs results in deletion of the IND from the double-stranded target DNA, e.g., the TRAC gene. In some embodiments, the target TRAC gene is a wild-type TRAC gene, and the IND is in any coding or non-coding region of the gene, the deletion or replacement of which results in abolishment or reduction in TRAC expression or function. In some embodiments, the IND is in a non-coding region of the TRAC gene. In some embodiments, the IND is in a coding region of the TRAC gene. In some embodiments, the IND is at a exon-intron junction, e.g., the exon 1-intron1 junction of the TRAC gene. In some embodiments, the IND position is designed such that after replacement of the IND by one or more recombinase recognition sequences (RSSs) encoded by the RD, potential exogenous transgene to be integrated mediated by a recombinase corresponding to the RSSs can be expressed using the endogenous promoter of the TRAC gene (although expression using an exogenous promoter, e.g. as part of the transgene, is always possible).

In certain embodiments, the first editing template of a first PEgRNA and the second editing template of a second PEgRNA comprise a region of complementarity to each other. In some embodiments, the first editing template encodes a first single stranded DNA, the second editing template encodes a second single stranded DNA, and the first and the second single stranded DNA comprise a region of complementarity to each other and are capable of forming a replacement duplex (RD). In certain embodiments, the region of complementarity between the first editing template and the second editing template comprises a sequence that is exogenous to the TRAC gene. In some embodiments, the RD comprises a sequence that is exogenous to the TRAC gene. In some embodiments, the exogenous sequence comprises one or more recombinase recognition sequences. In certain embodiments, the exogenous sequence may be a marker, expression tag, barcode or regulatory sequence. In certain embodiments, the region of complementarity between the first editing template and the second editing template comprises a nucleotide sequence that is at least partially identical to a sequence in the double-stranded target DNA or target gene. In certain embodiments, the region of complementarity between the first editing template and the second editing template comprises a nucleotide sequence that is at least partially identical to a sequence in the IND.

In some embodiments, the first editing template comprises a region of identity to the second spacer. In some embodiments, the first editing template comprises a region of identity to the second spacer and also comprises a region of complementarity to the second editing template. For example, the first editing template may comprise nucleotides 1-17, 2-17, 3-17, 4-17, 5-17, 6-7, 7-17, or 8-17 of the second spacer wherein the second spacer is 20nt in length. In some embodiments, the second editing template comprises a region of identity to the first spacer. In some embodiments, the second editing template comprises a region of identity to the first spacer and also comprises a region of complementarity to the first editing template. For example, the second editing template may comprise nucleotides 1-17, 2-17, 3-17, 4-17, 5-17, 6-7, 7-17, or 8-17 of the first spacer wherein the first spacer is 20nt in length.

In certain embodiments, the first editing template of a first PEgRNA and the second editing template of a second PEgRNA do not comprise a region of complementarity to each other. In certain embodiments, the first editing template of a first PEgRNA comprises region of identity to a sequence on the first target strand (or the first strand), and the second editing template comprises a region of identity to a sequence on the second target strand (or the second strand). In certain embodiments, the first editing template of a first PEgRNA comprises a region of identity to a sequence on the first target strand immediately adjacent to and outside the IND. In certain embodiments, the second editing template of a second PERNA comprises a region of identity to a sequence on the second target strand immediately adjacent to and outside the IND. In some embodiments, an editing template comprises one or more intended nucleotide edits to be incorporated in the double-stranded target DNA, e.g., the TRAC gene, by prime editing. In some embodiments, incorporation of the newly synthesized single-stranded DNA encoded by the editing template results in incorporation of one or more intended nucleotide edit in the double-stranded target DNA, e.g., the TRAC gene, compared to the endogenous sequence of the double-stranded target gene. For example, in some embodiments, the one or more intended nucleotide edits comprises deletion, insertion, and/or substitution of one or more nucleotides compared to the endogenous sequence of the double-stranded target gene, e.g., the TRAC gene.

In some embodiments, the editing template is a template for an RNA-dependent DNA polymerase domain or polypeptide of the prime editor, for example, a reverse transcriptase domain. The reverse transcriptase editing template may also be referred to herein as an RT template, or RTT.

In certain embodiments, the extension arm comprises a primer binding site sequence (PBS) that can initiate target-primed DNA synthesis. In some embodiments, the PBS is complementary or substantially complementary to a free 3′ end on the edit strand of the target gene at a nick site generated by the prime editor. In some embodiments, the first PEgRNA comprises a first PBS that comprises a region of complementarity to the second strand of the double-stranded target DNA or target gene. In some embodiments, the second PEgRNA comprises a second PBS that comprises a region of complementarity to the first strand of the double-stranded target DNA or target gene. In some embodiments, the first PEgRNA comprises a first PBS that comprises a region of complementarity to the first spacer of the first PEgRNA. In some embodiments, the first PEgRNA comprises a first PBS that is at least partially complementary to the first spacer of the first PEgRNA. In some embodiments, the second PEgRNA comprises a second PBS that comprises a region of complementarity to the second spacer of the second PEgRNA. In some embodiments, the second PEgRNA comprises a second PBS that is at least partially complementary to the second spacer of the second PERNA.

In some embodiments, a PEgRNA includes only RNA nucleotides and forms an RNA polynucleotide. In some embodiments, a PERNA is a chimeric polynucleotide that includes both RNA and DNA nucleotides. For example, a PEgRNA can include DNA in the spacer, the gRNA core, or the extension arm. In some embodiments, a PEgRNA comprises DNA in the spacer. In some embodiments, the entire spacer of a PEgRNA is a DNA sequence. In some embodiments, the PEgRNA comprises DNA in the gRNA core, for example, in a stem region of the gRNA core. In some embodiments, the PEgRNA comprises DNA in the extension arm, for example, in the editing template. An editing template that comprises a DNA sequence may serve as a DNA synthesis template for a DNA polymerase in a prime editor, for example, a DNA-dependent DNA polymerase. Accordingly, the PEgRNA may be a chimeric polynucleotide that comprises RNA in the spacer, gRNA core, and/or the PBS sequences and DNA in the editing template.

Components of a PEgRNA may be arranged in a modular fashion. In some embodiments, the spacer, the primer binding site sequence (PBS) and the editing template, e.g., a reverse transcriptase template (RTT), can be interchangeably located in the 5′ portion of the PEgRNA, the 3′ portion of the PERNA, or in the middle of the gRNA core. In some embodiments, a PEgRNA comprises a PBS and an editing template sequence in 5′ to 3′ order. In some embodiments, the gRNA core of a PEgRNA of this disclosure may be located in between a spacer and an extension arm (i.e., the PBS and editing template) of the PEgRNA. In some embodiments, the gRNA core of a PEgRNA may be located at the 3′ end of a spacer. In some embodiments, the gRNA core of a PERNA may be located at the 5′ end of a spacer. In some embodiments, the gRNA core of a PEgRNA may be located at the 3′ end of an extension arm. In some embodiments, the gRNA core of a PEgRNA may be located at the 5′ end of an extension arm. In some embodiments, a PEgRNA comprises, from 5′ to 3′: a spacer, a gRNA core, and an extension arm. In some embodiments, a PEgRNA comprises, from 5′ to 3′: a spacer, a gRNA core, an editing template, and a PBS. In some embodiments, a PEgRNA comprises, from 5′ to 3′: an extension arm, a spacer, and a gRNA core. In some embodiments, the PEgRNA comprises, from 5′ to 3′: an editing template a PBS, a spacer, and a gRNA core. In some embodiments, a first PEgRNA comprises a structure: 5′-[first spacer]-[first gRNA core]-[first editing template]-[first primer binding site sequence]-3′. In some embodiments, a first PEgRNA comprises a structure: 5′-[first editing template]-[first primer binding site sequence]-[first spacer]-[first gRNA core]-3′. In some embodiments, a second PERNA comprises a structure: 5′-[second spacer]-[second gRNA core]-[second editing template]-[second primer binding site sequence]-3′. In some embodiments, a second PEgRNA comprises a structure: 5′-[second editing template]-[second primer binding site sequence]-[second spacer]-[second gRNA core]-3′.

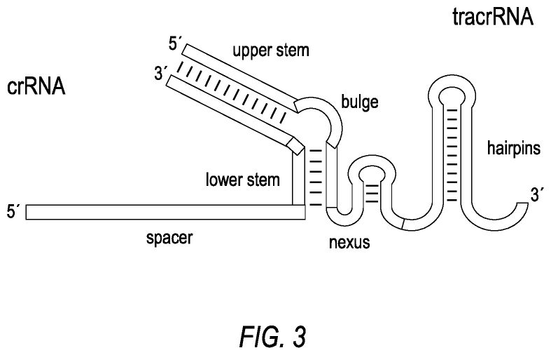

In some embodiments, a PEgRNA comprises a single polynucleotide molecule that comprises the spacer, the gRNA core, and the editing template. In some embodiments, a PEgRNA comprises a single polynucleotide molecule that comprises the spacer, the gRNA core, and the extension arm (i.e., a PBS and editing template). In some embodiments, a PEgRNA comprises multiple polynucleotide molecules, for example, two polynucleotide molecules. In some embodiments, a PEgRNA comprise a first polynucleotide molecule that comprises the spacer and a portion of the gRNA core, and a second polynucleotide molecule that comprises the rest of the gRNA core and the extension arm. In some embodiments, the gRNA core portion in the first polynucleotide molecule and the gRNA core portion in the second polynucleotide molecule are at least partly complementary to each other. In some embodiments, the PEgRNA may comprise a first polynucleotide comprising the spacer and a first portion of a gRNA core referred to as a crRNA. In some embodiments, the PERNA comprise a second polynucleotide comprising a second portion of the gRNA core and the extension arm, wherein the second portion of the gRNA core may also be referred to as a trans-activating crRNA, or tracr RNA. In some embodiments, the crRNA portion and the tracr RNA portion of the gRNA core are at least partially complementary to each other. In some embodiments, the partially complementary portions of the crRNA and the tracr RNA form a lower stem, a bulge, and an upper stem, as exemplified in FIG. 3.

In some embodiments, a first spacer comprises a region that has substantial complementarity to a first search target sequence on a first target strand, or first strand, of a double-stranded target DNA, e.g., a TRAC gene. In some embodiments, the first spacer of a PERNA is identical or substantially identical to a protospacer sequence on the second strand of the target gene (except that the protospacer sequence comprises thymine and the spacer may comprise uracil). In some embodiments, the first spacer is at least about 70%, 75%, 80%, 85%, 90%, 95%, or 100% complementary to a first search target sequence in the target gene. In some embodiments, the first spacer is substantially complementary to the first search target sequence. In some embodiments, a second spacer comprises a region that has substantial complementarity to a second search target sequence on the second target strand, or the second strand, of a double-stranded target DNA, e.g., a TRAC gene. In some embodiments, the second spacer of a PEgRNA is identical or substantially identical to a protospacer on the first strand of the target gene (except that the protospacer comprises thymine and the spacer may comprise uracil). In some embodiments, the second spacer is at least about 70%, 75%, 80%, 85%, 90%, 95%, or 100% complementary to a second search target sequence in the target gene. In some embodiments, the second spacer is substantially complementary to the second search target sequence.

In some embodiments, the length of the spacer varies from at least 10 nucleotides to 100 nucleotides. For examples, a spacer may be at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides. In some embodiments, the spacer is 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides in length. In some embodiments, the spacer is from 15 nucleotides to 30 nucleotides in length, 15 to 25 nucleotides in length, 17 to 23 nucleotides in length, 18 to 22 nucleotides in length, 10 to 20 nucleotides in length, 20 to 30 nucleotides in length, 30 to 40 nucleotides in length, 40 to 50 nucleotides in length, 50 to 60 nucleotides in length, 60 to 70 nucleotides in length, 70 to 80 nucleotides in length, or 90 nucleotides to 100 nucleotides in length. In some embodiments, the spacer is 20 nucleotides in length. In some embodiments, the spacer is 17 to 18 nucleotides in length. In some embodiments, the spacer is 21 to 22 nucleotides in length.

In some embodiments, a PEgRNA sequence may further comprise additional nucleotides the precede a spacer beside a region of complementarity to genomic search target sequence. For example, in some embodiments, a PEgRNA sequence (as well as the full PEgRNA sequence) may comprise an additional G at the 5′ end, for example, wherein the 5′ most nucleotide of the spacer (or the PEgRNA) is not a G. The addition of the G at the 5′ end of the PEgRNA where the PEgRNA does not already begin with a G may enable transcription from a U6 promoter. Such an adaptation may be referred to as a transcription adaptation.

The extension arm of a first PEgRNA may be partially complementary to the spacer of the first PEgRNA. In some embodiments, the editing template (e.g., RTT) of a first PEgRNA is partially complementary to the spacer of the first PEgRNA. In some embodiments, the editing template (e.g., RTT) and the primer binding site (PBS) of the first PEgRNA are each partially complementary to the spacer of the first PEgRNA. The extension arm of a PEgRNA may comprise a primer binding site (PBS) and an editing template (e.g., an RTT). The extension arm of a second PEgRNA may be partially complementary to the spacer of the second PEgRNA. In some embodiments, the editing template (e.g., RTT) of a second PERNA is partially complementary to the spacer of the second PEgRNA. In some embodiments, the editing template (e.g., RTT) and the primer binding site (PBS) of the second PEgRNA are each partially complementary to the spacer of the second PEgRNA.

An extension arm of a PEgRNA may comprise a primer binding site sequence (also referred to as a primer binding site, PBS, or PBS sequence) that hybridizes with a free 3′ end of a single-stranded DNA in the target gene (e.g., the TRAC gene) generated by nicking with a prime editor. The length of the PBS sequence may vary depending on, e.g., the prime editor components, the search target sequence and other components of the PERNA. In some embodiments, the length of the primer binding site (PBS) varies from at least 2 nucleotides to 50 nucleotides. For examples, a primer binding site (PBS) may be at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, or at least 50 nucleotides in length. In some embodiments, the PBS is at least 4 nucleotides in length. In some embodiments, the PBS is at least 6 nucleotides in length. In some embodiments, the PBS is about 4 to 12 nucleotides, about 6 to 12 nucleotides, about 8 to 12 nucleotides, about 10 to 12 nucleotides, 4 to 14 nucleotides, about 6 to 14 nucleotides, about 8 to 14 nucleotides, about 10 to 14 nucleotides, about 12 to 14 nucleotides, 4 to 16 nucleotides, about 6 to 16 nucleotides, about 8 to 16 nucleotides, about 10 to 16 nucleotides, about 6 to 18 nucleotides, about 6 to 20 nucleotides, about 8 to 20 nucleotides, about 10 to 20 nucleotides, about 12 to 20 nucleotides, about 14 to 20 nucleotides, about 16 to 20 nucleotides, or about 18 to 20 nucleotides in length. In some embodiments, the PBS is about 7 to 15 nucleotides in length. In some embodiments, the PBS is 6, 7, 8, 9, 10, 11, 12, 13, 14, 15, 16, 17, 18, 19, or 20 nucleotides in length. In some embodiments, the PBS is 8, 9, 10, 11, 12, 13, or 14 nucleotides in length.

The PBS of a first PEgRNA may be complementary or substantially complementary to a DNA sequence in the second strand of the target gene. The PBS of a second PEgRNA may be complementary or substantially complementary to a DNA sequence in the first strand of the target gene. By annealing with the edit strand at a free hydroxy group, e.g., a free 3′ end generated by prime editor nicking, a PBS may initiate synthesis of a new single-stranded DNA encoded by the editing template at the nick site. In some embodiments, a PBS is at least about 70%, 75%, 80%, 85%, 90%, 95%, or 100% complementary to a region of the edit strand of the target gene (e.g., the TRAC gene). In some embodiments, a PBS is perfectly complementary, or 100% complementary, to a region of the edit strand of the target gene (e.g., the TRAC gene).

Exemplary sequences for PBS are provided in Tables 9-25.

An extension arm of a PEgRNA may comprise an editing template that serves as a DNA synthesis template for the DNA polymerase in a prime editor during prime editing.

The length of an editing template may vary depending on, e.g., the prime editor components, the search target sequence and other components of the PEgRNA. In some embodiments, the editing template serves as a DNA synthesis template for a reverse transcriptase, and the editing template is referred to as a reverse transcription editing template or simply reverse transcriptase template (RTT).

In some embodiments, the editing template (e.g., RTT) is 2, 3, 4, 5, 6, 7, 8, 9, 10, 11, 12, 13, 14, 15, 16, 17, 18, 19, 20, 21, 22, 23, 24, or 25 nucleotides in length. In some embodiments, the editing template is 12, 13, 14, 15, 16, 17, 18, 19, 20, 21, 22, 23, 24, or 25 nucleotides in length. In some embodiments, the editing template is 16, 17, 18, 19, 20, 21, 22, 23, 24, 25, 26, 27, 28, 29, 30, 31, 32, 33, 34, 35, 36, 37, 38, 39, or 40 nucleotides in length. In some embodiments, the editing template comprises 2, 3, 4, 5, 6, 7, 8, 9, 10, 11, 12, 13, 14, 15, 16, 17, 18, 19, 20, 21, 22, 23, 24, 25, 30, 35, 40, 45, 50 or more nucleotides. In some embodiments, the editing template comprises about 5 to 10, 5 to 15, 5 to 20, 5 to 25, 5 to 30, 5 to 35, 5 to 40, 5 to 45, 5 to 50, 5 to 55, 5 to 60, 5 to 65, 5 to 70, 5 to 75, 5 to 80, 5 to 85, 5 to 90, 5 to 95, 5 to 100, 5 to 110, 5 to 120, 5 to 130, 5 to 140, 5 to 150, 15 to 20, 15 to 25, 15 to 30, 15 to 35, 15 to 40, 15 to 45, 15 to 50, 15 to 55, 15 to 60, 15 to 65, 15 to 70, 15 to 75, 15 to 80, 15 to 85, 15 to 90, 15 to 95, 15 to 100, 15 to 110, 15 to 120, 15 to 130, 15 to 140, 15 to 150, 25 to 30, 25 to 35, 25 to 40, 25 to 45, 25 to 50, 25 to 55, 25 to 60, 25 to 65, 25 to 70, 25 to 75, 25 to 80, 25 to 85, 25 to 90, 25 to 95, 25 to 100, 25 to 110, 25 to 120, 25 to 130, 25 to 140, 25 to 150, 35 to 40, 35 to 45, 35 to 50, 35 to 55, 35 to 60, 35 to 65, 35 to 70, 35 to 75, 35 to 80, 35 to 85, 35 to 90, 35 to 95, 35 to 100, 35 to 110, 35 to 120, 35 to 130, 35 to 140, 35 to 150, 45 to 50, 45 to 55, 45 to 60, 45 to 65, 45 to 70, 45 to 75, 45 to 80, 45 to 85, 45 to 90, 45 to 95, 45 to 100, 45 to 110, 45 to 120, 45 to 130, 45 to 140, o45 to 150, 55 to 60, 55 to 65, 55 to 70, 55 to 75, 55 to 80, 55 to 85, 55 to 90, 55 to 95, 55 to 100, 55 to 110, 55 to 120, 55 to 130, 55 to 140, 55 to 150, 65 to 70, 65 to 75, 65 to 80, 65 to 85, 65 to 90, 65 to 95, 65 to 100, 65 to 110, 65 to 120, 65 to 130, 65 to 140, 65 to 150, 75 to 80, 75 to 85, 75 to 90, 75 to 95, 75 to 100, 75 to 110, 75 to 120, 75 to 130, 75 to 140, 75 to 150, 85 to 90, 85 to 95, 85 to 100, 85 to 110, 85 to 120, 85 to 130, 85 to 140, 85 to 150, 95 to 100, 95 to 110, 95 to 120, 95 to 130, 95 to 140, 95 to 150, 105 to 110, 105 to 120, 105 to 130, 105 to 140, 105 to 150, 115 to 120, 115 to 130, 115 to 140, 115 to 150, 125 to 130, 125 to 140, 125 to 150, 135 to 140, 135 to 150, or 145 to 150 nucleotides in length.

In some embodiments, the editing template comprises 10, 11, 12, 13, 14, 15, 16, 17, 18, 19, 20, 21, 22, 23, 24 or 25 nucleotides in length. In some embodiments, the editing template comprises 30, 35, 40, 50, 60, 70, 80, 90, or 100 nucleotides in length. In some embodiments, the editing template comprises no greater than 20, 25, 30, 35, 40, 50, 60, 70, 80, 90, or 100 nucleotides in length. In some embodiments, the editing template comprises a sufficient number of nucleotides to form a sufficiently stable duplex with a sequence on the double-stranded target DNA. In some embodiments, the editing template comprises at least 10 polynucleotides. In some embodiments, the editing template comprises at least 15 polynucleotides. In some embodiments, the editing template comprises at least 20 polynucleotides.

In some embodiments, an editing template may include sequences unrelated to the endogenous sequence. Such editing templates (RTTs) can enable insertion of a readily-identifiable sequence to permit rapid determination of successful editing, or can be used to improve editing efficiency by controlling insert length or GC content.

In some embodiments, an editing template may encode one or more recombinase recognition sequences (RSSs). Such editing templates, or RTTs, can enable insertion of the RSS(s) in the target gene, and the RSS(s) can be used as landing sites for recombinase mediated DNA insertion, deletion, inversion, or replacement. For example, in some embodiments, prime editing insertion of a RSS (e.g., attB sequence) allows integration of a DNA donor sequence mediated by a recombinase that recognizes the RSS (e.g., Bxb1), wherein the DNA donor sequence also comprises a RSS recognized by the recombinase (e.g., attP sequence). In some embodiments, prime editing insertion of two RSSs allow for deletion of the target gene sequence between the two RSSs or inversion of the target gene sequence between the two RSSs mediated by a recombinase that recognizes the two RSSs, depending on the orientation of the two RSSs. In some embodiments, prime editing insertion of two RSSs allow for cassette exchange of the target gene sequence between the two RSSs and a DNA donor sequence mediated by a corresponding recombinase, wherein the DNA donor sequence is flanked by two RSSs that are also recognized by the recombinase.

Exemplary RSS sequences that can be encoded by the PEgRNA or dual PEgRNA RTTs are provided in Table 5. A skilled person understands that RSSs recognized by the same recombinase can be used for targeted insertion and other recombination events, for example, by inserting an attB sequence in a target TRAC gene via prime editing, and providing a Bxb1 recombinase and a circular DNA donor construct containing an attP sequence for integration of the DNA donor sequence in the TRAC gene at the attB site. In some embodiments, orthogonal recognition can be achieved by altering the central dinucleotide of the RSS. For example, the central dinucleotides of Bxb1 attB or attP sequences can be GT or GA, shown in bold in Table 5. In some embodiments, the central dinucleotides of a RSS can be any two nucleotides, where each nucleotide is A, T, G, or C. Additional RSSs described herein and those known in the art, as well as corresponding recombinases, are also contemplated.

TABLE 5
Exemplary RSS sequences and corresponding recombinases

| Recombinase | | |
|---|---|---|
| recognition | | Corresponding |
| sequence | DNA sequence | recombinase |
| Bxb1 attB | GGCTTGTCGACGACGGCGGTCTCCGTCGTCAGGATCAT | Bxb1 |
| | (SEQ ID NO: 1187) | |
| Bxb1 attP | GGTTTGTCTGGTCAACCACCGCGGTCTCAGTGGTGTACGG | Bxb1 |
| | TACAAACC (SEQ ID NO: 1188) | |
| Bxb1 attB-GA | GGCTTGTCGACGACGGCGGACTCCGTCGTCAGGATCAT | Bxb1 |
| | (SEQ ID NO: 1189) | |
| Bxb1 attP-GA | GGTTTGTCTGGTCAACCACCGCGGACTCAGTGGTGTACG | Bxb1 |
| | GTACAAACC (SEQ ID NO: 1190) | |
| Pa01 attB | CCGGTTTCCCTTCGCACCCGCACCGCGGCTTCGAGACCGT | Pa01 |
| | GACCTACATGCTCGAAGGGCGTATGCGCCACGAAGACCA | |
| | CCTCGGCAATCGCGGCCTGCTCAAG (SEQ ID NO: 1191) | |
| Pa01 | GGTTTGTCTGGTCAACCACCGCGGACTCAGTGGTGTACGG | Pa01 |
| attB_39nt | TACAAACC (SEQ ID NO: 1192) | |
| Pa01 | CCGTGACCTACATGCTCGAAGGGCGTATGCGCC (SEQ ID | Pa01 |
| attB_33nt | NO: 1193) | |
| Pa01 attP | GTAACGCTCTTCGAGAAAGCAGATTCTCATATCCATCTTG | Pa01 |
| | AGTCTTCTTTCTCGCAAGACAACACGAAATAGACACAGT | |
| | CTCTTCCCTAGCTGTACACTGAGCC (SEQ ID NO: 1194) | |
| Si74 attB | TTCCGACGCAGTTTCCGACGAGTACGAGGACGAGGACAG | Si74 |
| | ACGTGCCTACCGGCAAGGTCAAGTGGTTCAACAGCGAGA | |
| | AGGGCTTCGGCTTTCTCTCCCGCGACGACGGCGG (SEQ ID | |
| | NO: 1195) | |
| Si74 attP | TAGTGACGTCTGTCCGCGCAGTGATCGAGGGAGTGTGTG | Si74 |
| | CTTTGCCGACTGGCAAGGTCAAGCCGGTCTGCTAGGCAC | |
| | AGAGAGCCGGTACAGTCCTCCCCATGCAACCCAA (SEQ ID | |
| | NO: 1196) | |
| No67 attB | GTGACCCGTTCCGCCGTCGAGAACGCCACCTCCGTGGCG | No67 |
| | CGCATGGTGCTCACCACCGAGAGCGCCGTGGTCGACAAG | |
| | CCGGCCGAGGAAGAGCCCGCGAACGGCCACCACGGCC | |
| | (SEQ ID NO: 1197) | |
| No67 attP | TGATTACGATCAGTGCCCTGGGAGGCGATTCCGGCATGG | No67 |
| | CTTATATCCAACACCACCGAGAGCGCTGTTGTCGAGCGTG | |
| | TAAGCCAGGACGAGGACGAGCACGCCCACGGGCACG | |
| | (SEQ ID NO: 1198) | |
| Kp03 attB | TTTGCATTTAAAATCGGAGCATCATTTTTCAACAGAAACG | Kp03 |
| | ACTATGAGCGCAATGACCCTTGATTTACCTCGCCGCTTTC | |
| | CGTGGCCAACGCTGCTGTCCGTGGCTATCCACG (SEQ ID | |
| | NO: 1199) | |
| Kp03 attP | AGAACCTTGAAAAACTATGGCTTATGCTACCTCGCCGAAT | Kp03 |
| | AGCTCAAATACAATGACCCTTGAATATAGGCTATTCGGGT | |
| | TTTGAAGGTATCTGGTTTTTATTCACATCACAA (SEQ ID | |
| | NO: 1200) | |
| Nm60 attB | CGAAATGGTTGGCGTTGAGGTCAATGATTAATGTGTATA | Nm60 |
| | GGGTTAACATTTAAATCAGTACAATCGTAGACGCTCTACA | |
| | CTATTTTCTGTGTATAAAAATATCGAGAATAAACGCTT | |
| | (SEQ ID NO: 1201) | |
| Nm60 attP | GCTGGTCGAGGAGTTAATGAAGATCTCCAACATGTTCGG | Nm60 |
| | CTACGGTGAGGTAAATCAGTACAATCGTCACTCGACCCT | |
| | AATGTCTGCCTATAAAAACATCAAAGATGAACATTTCCG | |
| | (SEQ ID NO: 1202) | |
| BceINTa attB | GATATGGGGAAGTGAATCAGTACAACCGCCACAGTACC | BceINTa |
| | (SEQ ID NO: 1203) | |
| BceINTa attP | TTCGGGTGCTGGGTTGTTGTCTCTGGACAGTGATCCATGG | BceINTa |
| | GAAACTACTCAGCACCA (SEQ ID NO: 1204) | |
| BcytINTd | ATTGTTGTTGTTTTTCCAGATCCAGTTGGTCCTGTAAATAT | BcytINTd |
| attB | AAGCAATCC (SEQ ID NO: 1205) | |
| BcytINTd attP | GGTTGTATTTGTAGAACTTGACCAGTTGTTTTAGTAACAT | BcytINTd |
| | AAATACAACT (SEQ ID NO: 1206) | |
| SscINTd attB | CATTATATGTTTTTACAATCCGGGCCGCCATACTGTAAGA | SscINTd |
| | ACATATAATG (SEQ ID NO: 1207) | |
| SscINTd attP | GATATGGGGAAGTGAATcaGTACAACCGCCACAGTACC | SscINTd |
| | (SEQ ID NO: 1208) | |
| SacINTd attB | CGTTATAGGGTATTGCAGTACCGACCGCCATACTGTAATA | SacINTd |
| | CCCTATAACG (SEQ ID NO: 1209) | |
| SacINTd attP | GATGCACTGAGCTCACCGTCCGGACCGCCATACTGACTTA | SacINTd |
| | TGATATAAGA (SEQ ID NO: 1210) | |

Exemplary sequences that can be included in the first editing template and the second editing template, or vice versa, for insertion of a recombinase recognition sequence into a target gene are provided in Table 6. The sequences in Table 6 are organized in pairs such that each paired sequences have a region of reverse complementarity to each other. Each pair of editing templates in Table 6 is assigned a RTT Pair Number. In some embodiments, the first editing template can be based on either of the sequences in a given pair so long as the second editing template is based upon the other sequence in the pair. The editing templates can comprise a complete sequence listed in Table 6. The editing templates can also comprise any shorter 5′ fragments of the sequences in Table 6 so long as at least 10 nucleotides at the 5′ ends of the first and second editing templates have perfect reverse complementarity to each other. In some embodiments, the 5′ ends of the first and second editing templates have at least 15 nucleotides of perfect reverse complementarity to each other. In some embodiments, the 5′ ends of the first and second editing templates have at least 20 nucleotides of perfect reverse complementarity to each other. In some embodiments, the first editing template can be any one of the RTT #1 having a RTT pair number 2 or 3 wherein the second editing template is any one of the RTT #2 having a RTT pair number 2 or 3. In some embodiments, the first editing template can be any one of the RTT #2 having a RTT pair number 2 or 3 wherein the second editing template is any one of the RTT #1 having a RTT pair number 2 or 3.

As used herein in a PEgRNA sequence, or fragments thereof such as a spacer, PBS, or RTT sequence, unless indicated otherwise, it should be appreciated that the letter “T” or “thymine” indicates a nucleobase in a DNA sequence that encodes the PEgRNA or guide RNA sequence, and is intended to refer to a uracil (U) nucleobase of the PEgRNA or guide RNA or any chemically modified uracil nucleobase known in the art, such as 5-methoxyuracil.

TABLE 6
Exemplary editing template (RTT) sequences. Although all the sequences
provided in Table 6 are RNA sequences, “T” is used instead of a “U” in the
sequences for consistency with the ST.26 standard.

| Description | Sequence | RTT Pair No. |
|---|---|---|
| Bxb1_29 nt_RTT#1 | ACGACGGAGACCGCCGTCGTCGACAAGCC (SEQ ID NO: 23) | 1 |
| Bxb1_30 nt_RTT#1 | GACGACGGAGACCGCCGTCGTCGACAAGCC (SEQ ID NO: 24) | 1 |
| Bxb1_38 nt_RTT#1 | ATGATCCTGACGACGGAGACCGCCGTCGTCGACAAGCC (SEQ | 1 |
| | ID NO: 27) | |
| Pa01_27 nt_RTT#1 | TACGCCCTTCGAGCATGTAGGTCACGG (SEQ ID NO: 22) | 2 |
| Pa01_30 nt_RTT#1 | GCATACGCCCTTCGAGCATGTAGGTCACGG (SEQ ID NO: 25) | 3 |
| Pa01_33 nt_RTT#1 | GGCGCATACGCCCTTCGAGCATGTAGGTCACGG (SEQ ID | 2 |
| | NO: 26) | |
| Pa01_39 nt_RTT#1 | CTTCGTGGCGCATACGCCCTTCGAGCATGTAGGTCACGG | 3 |
| | (SEQ ID NO: 28) | |
| Bxb1_29 nt_RTT#2 | ACGACGGCGGTCTCCGTCGTCAGGATCAT (SEQ ID NO: 196) | 1 |
| Bxb1_30 nt_RTT#2 | GACGACGGCGGTCTCCGTCGTCAGGATCAT (SEQ ID NO: 106) | 1 |
| Bxb1_38 nt_RTT#2 | GGCTTGTCGACGACGGCGGTCTCCGTCGTCAGGATCAT (SEQ | 1 |
| | ID NO: 107) | |
| Pa01_27 nt_RTT#2 | CCTACATGCTCGAAGGGCGTATGCGCC (SEQ ID NO: 195) | 2 |
| Pa01_30 nt_RTT#2 | ACATGCTCGAAGGGCGTATGCGCCACGAAG (SEQ ID NO: 197) | 3 |
| Pa01_33 nt_RTT#2 | CCGTGACCTACATGCTCGAAGGGCGTATGCGCC (SEQ ID | 2 |
| | NO: 198) | |
| Pa01_39 nt_RTT#2 | CCGTGACCTACATGCTCGAAGGGCGTATGCGCCACGAAG | 3 |
| | (SEQ ID NO: 199) | |
| Bxb1_30 nt_RTT#2 | GACGACGGAGACCGCCGTCGTCGACAAGCC (SEQ ID NO: 24) | 1 |

An intended nucleotide edit in an editing template of a PEgRNA may comprise various types of alterations as compared to the target gene sequence. In some embodiments, the nucleotide edit is a single nucleotide substitution as compared to the target gene sequence. In some embodiments, the nucleotide edit is a deletion as compared to the target gene sequence. In some embodiments, the nucleotide edit is an insertion as compared to the target gene sequence. In some embodiments, the editing template comprises one to ten intended nucleotide edits as compared to the target gene sequence. In some embodiments, the editing template comprises one or more intended nucleotide edits as compared to the target gene sequence. In some embodiments, the editing template comprises two or more intended nucleotide edits as compared to the target gene sequence. In some embodiments, the editing template comprises three or more intended nucleotide edits as compared to the target gene sequence. In some embodiments, the editing template comprises four or more, five or more, or six or more intended nucleotide edits as compared to the target gene sequence. In some embodiments, the editing template comprises two single nucleotide substitutions, insertions, deletions, or any combination thereof, as compared to the target gene sequence. In some embodiments, the editing template comprises three single nucleotide substitutions, insertions, deletions, or any combination thereof, as compared to the target gene sequence. In some embodiments, the editing template comprises four, five, or six single nucleotide substitutions, insertions, deletions, or any combination thereof, as compared to the target gene sequence. In some embodiments, a nucleotide substitution comprises an adenine (A)-to-thymine (T) substitution. In some embodiments, a nucleotide substitution comprises an A-to-guanine (G) substitution. In some embodiments, a nucleotide substitution comprises an A-to-cytosine (C) substitution. In some embodiments, a nucleotide substitution comprises a T-A substitution. In some embodiments, a nucleotide substitution comprises a T-G substitution. In some embodiments, a nucleotide substitution comprises a T-C substitution. In some embodiments, a nucleotide substitution comprises a G-to-A substitution. In some embodiments, a nucleotide substitution comprises a G-to-T substitution. In some embodiments, a nucleotide substitution comprises a G-to-C substitution. In some embodiments, a nucleotide substitution comprises a C-to-A substitution. In some embodiments, a nucleotide substitution comprises a C-to-T substitution. In some embodiments, a nucleotide substitution comprises a C-to-G substitution.

In some embodiments, a nucleotide insertion is at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, or at least 20 nucleotides in length. In some embodiments, a nucleotide insertion is from 1 to 2 nucleotides, from 1 to 3 nucleotides, from 1 to 4 nucleotides, from 1 to 5 nucleotides, form 2 to 5 nucleotides, from 3 to 5 nucleotides, from 3 to 6 nucleotides, from 3 to 8 nucleotides, from 4 to 9 nucleotides, from 5 to 10 nucleotides, from 6 to 11 nucleotides, from 7 to 12 nucleotides, from 8 to 13 nucleotides, from 9 to 14 nucleotides, from 10 to 15 nucleotides, from 11 to 16 nucleotides, from 12 to 17 nucleotides, from 13 to 18 nucleotides, from 14 to 19 nucleotides, from 15 to 20 nucleotides in length. In some embodiments, a nucleotide insertion is a single nucleotide insertion. In some embodiments, a nucleotide insertion comprises insertion of two nucleotides.

The editing template of a PEgRNA may comprise one or more intended nucleotide edits, compared to the TRAC gene to be edited. Position of the intended nucleotide edit(s) relevant to other components of the PEgRNA, or to particular nucleotides (e.g., mutations) in the TRAC target gene may vary. In some embodiments, the nucleotide edit is in a region of the PEgRNA corresponding to or homologous to the protospacer sequence. In some embodiments, the nucleotide edit is in a region of the PEgRNA corresponding to a region of the TRAC gene outside of the protospacer sequence. In some embodiments, incorporation of the one or more intended nucleotide edits in the target TRAC gene results in a missense mutation, a nonsense mutation, a frame-shift mutation, a null mutation, or a mutation that generates a premature stop codon in the target TRAC gene that abolishes or decreases TRAC expression. In some embodiments, the one or more intended nucleotide edits introduce a frame shift mutation and/or generate one or more premature stop codons (e.g., at least 1, 2, 3, 4, 5, or more premature stop codons) in the target gene (e.g., TRAC gene). In some embodiments, the one or more intended nucleotide edits generates at least 2 premature stop codons in the target gene. In some embodiments, the one or more intended nucleotide edits generates at least 2, 3, 4, 5, or more consecutive premature stop codons in the target gene (e.g., a TRAC gene). In some embodiments, incorporation of the one or more intended nucleotide edits introduces one or more recombinase sites in the TRAC gene. In some embodiments, incorporation of the one or more intended nucleotide edits results in deletion of the IND from the double-stranded target DNA, e.g., the TRAC gene. In some embodiments, incorporation of the one or more intended nucleotide edits results in deletion of the IND from, and insertion of an exogenous sequence encoded by the editing templates into the TRAC gene. In some embodiments, the exogenous sequence contains one or more RSSs, e.g., an attB sequence or an attP sequence.

In some embodiments, the editing template comprises an adenine at the first nucleobase position (e.g., for a PEgRNA following 5′-spacer-gRNA core-RTT-PBS-3′ orientation, the 5′ most nucleobase is the “first base”). In some embodiments, the editing template comprises a guanine at the first nucleobase position (e.g., for a PERNA following 5′-spacer-gRNA core-RTT-PBS-3′ orientation, the 5′ most nucleobase is the “first base”). In some embodiments, the editing template comprises a uracil at the first nucleobase position (e.g., for a PEgRNA following 5′-spacer-gRNA core-RTT-PBS-3′ orientation, the 5′ most nucleobase is the “first base”). In some embodiments, the editing template comprises a cytosine at the first nucleobase position (e.g., for a PERNA following 5′-spacer-gRNA core-RTT-PBS-3′ orientation, the 5′ most nucleobase is the “first base”). In some embodiments, the editing template does not comprise a cytosine at the first nucleobase position (e.g., for a PEgRNA following 5′-spacer-gRNA core-RTT-PBS-3′ orientation, the 5′ most nucleobase is the “first base”).

A guide RNA core (also referred to herein as the gRNA core, gRNA scaffold, or gRNA backbone sequence) of a PEgRNA may contain a polynucleotide sequence that binds to a DNA binding domain (e.g., Cas9) of a prime editor. The gRNA core may interact with a prime editor as described herein, for example, by association with a DNA binding domain, such as a Cas9 nickase of the prime editor.

One of skill in the art will recognize that different prime editors having different DNA binding domains from different DNA binding proteins may require different gRNA core sequences specific to the DNA binding protein. In some embodiments, the gRNA core is capable of binding to a Cas9-based prime editor. In some embodiments, the gRNA core is capable of binding to a Cpf1-based prime editor. In some embodiments, the gRNA core is capable of binding to a Cas12b-based prime editor.

In some embodiments, the gRNA core comprises regions and secondary structures involved in binding with specific CRISPR Cas proteins. For example, in a Cas9 based prime editing system, the gRNA core of a PEgRNA may comprise one or more regions of a base paired “lower stem” adjacent to the spacer and a base paired “upper stem” following the lower stem, where the lower stem and upper stem may be connected by a “bulge” comprising unpaired RNAs. The gRNA core may further comprise a “nexus” distal from the spacer, followed by a hairpin structure, e.g., at the 3′ end, as exemplified in FIG. 3. In some embodiments, the gRNA core comprises modified nucleotides as compared to a wild-type gRNA core in the lower stem, upper stem, and/or the hairpin. For example, nucleotides in the lower stem, upper stem, and/or the hairpin regions may be modified, deleted, or replaced. In some embodiments, RNA nucleotides in the lower stem, upper stem, and/or the hairpin regions may be replaced with one or more DNA sequences. In some embodiments, the gRNA core comprises unmodified or wild-type RNA sequences in the nexus and/or the bulge regions. In some embodiments, the gRNA core does not include long stretches of A-T pairs, for example, a GUUUU-AAAAC pairing element. Exemplary gRNA cores sequences are found in Table 7, below.

TABLE 7
Exemplary gRNA core sequences. Although all the sequences
provided in Table 7 are RNA sequences, “T” is used instead
of a “U” in the sequences for consistency with the
ST.26 standard.

| SEQ ID NO: | gRNA core Sequence |
|---|---|
| 590 | GTTTTAGAGCTAGAAATAGCAAGTTAAAATAAGGCTAGTCCGTTAT |
| | CAACTTGAAAAAGTGGCACCGAGTCGGTGC |
| 604 | GTTTAAGAGCTAGAAATAGCAAGTTTAAATAAGGCTAGTCCGTTAT |
| | CAGCGTGAAAACGCGGCACCGAGTCGGTGC |
| 605 | GTTTAAGAGCTATGCTGGAAACAGCATAGCAAGTTTAAATAAGGCT |
| | AGTCCGTTATCAACTTGAAAAAGTGGCACCGAGTCGGTGC |
| 606 | GTTTGAGAGCTAGAAATAGCAAGTTTAAATAAGGCTAGTCCGTTAT |
| | CAACTTGAAAAAGTGGGACCGAGTCGGTCC |
| 607 | GTTTAAGAGCTAGAAATAGCAAGTTTAAATAAGGCTAGTCCGTTAT |
| | CAACTTGAAAAAGTGGCACCGAGTCGGTGC |
| 608 | GTTTTAGAGCTAGCTCATGAAAATGAGCTAGCAAGTTAAAATAAG |
| | GCTAGTCCGTTATCAACTTGAAAAAGTGGGACCGAGTCGGTCC |
| 609 | GTTTTAGAGCTATACGTAGCAAGTTAAAATAAGGCTAGTCCGTTAT |
| | CAACTTTACGAAGTGGCACCGAGTCGGTGC |
| 610 | GTTTTAGAGCTATACGTAGCAAGTTAAAATAAGGCTAGTCCGTTAT |
| | CAACTTTACGAAGTGGGACCGAGTCGGTCC |
| 611 | GTTTTAGAGCTATGCTGGAAACAGCATAGCAAGTTAAAATAAGGCT |
| | AGTCCGTTATCAACTTGAAAAAGTGGCACCGAGTCGGTGC |

In some embodiments, the gRNA core comprises SEQ ID NO: 590. In some embodiments, the gRNA core comprises SEQ ID NO: 604.

In some embodiments, a PEgRNA comprises a gRNA core that comprises a modified direct repeat compared to the sequence of a naturally occurring CRISPR-Cas guide RNA scaffold, for example, a Cas9 gRNA scaffold. In some embodiments, the PEgRNA comprises a “flip and extension (F+E)” gRNA core, wherein one or more base pairs in a direct repeat is modified. In some embodiments, the PEgRNA comprises a first direct repeat (the first paring element or the lower stem), wherein a uracil is changed to an adenine (such that in the stem region, a U-A base pair is changed to an A-U base pair). In some embodiments, the PEgRNA comprises a first direct repeat wherein the fourth U-A base pair in the stem is changed to an A-U base pair. In some embodiments, the PEgRNA comprises a first direct repeat wherein one or more U-A base pair is changed to a G-C or C-G base pair. For example, in some embodiments, the PEgRNA comprises a first direct repeat comprising a modification to a GUUUU-AAAAC pairing element, wherein one or more of the U-A base pairs is changed to an A-U base pair, a G-C base pair, or a C-G base pair. In some embodiments, the PEgRNA comprises an extended first direct repeat.

Any gRNA core sequences known in the art are also contemplated in the prime editing compositions described herein. In some embodiments, one or more nucleotides in the gRNA core is DNA.

In some embodiments, a PEgRNA comprises an additional secondary structure at the 5′ end. In some embodiments, a PEgRNA comprises an additional secondary structure at the 3′ end. Nucleotide sequences for exemplary secondary structure motifs for PEgRNAs are found in Table 8. Table 8. Exemplary PEgRNA secondary structure motifs. Although all the sequences provided in

TABLE 8
are RNA sequences, “T” is used instead of a “U” in the sequences
for consistency with the ST.26 standard.

| SEQ ID NO: | Secondary Structure Motif Sequence |
|---|---|
| 612 | GGGTCAGGAGCCCCCCCCCTGAACCCAGGATAACCCTCAAAGTCGGGG |
| | GGCAACC |
| 613 | GTCAGGGTCAGGAGCCCCCCCCCTGAACCCAGGATAACCCTCAAAGTC |
| | GGGGGGCAACCC |
| 614 | GGGTCAGGAGCCCCCCCCCTGAACCCAGGAAAACCCTCAAAGTCGGGG |
| | GGCAACCC |
| 615 | GGGTCAGGAGCCCCCCCCCTGCACCCAGGAAAACCCTCAAAGTCGGGG |
| | GGCAACCC |
| 616 | GGGTCAGGAGCCCCCCCCCTGCACCCAGGATAACCCTCAAAGTCGGGG |
| | GGCAACCC |
| 617 | GTCAGGGTCAGGAGCCCCCCCCCTGAACCCAGGAAAACCCTCAAAGTC |
| | GGGGGGCAACCC |
| 618 | GTCAGGGTCAGGAGCCCCCCCCCTGCACCCAGGAAAACCCTCAAAGTC |
| | GGGGGGCAACCC |
| 619 | GGGTCAGGAGCCCCCCCCCTGAACCCAGGATAACCCTCAAAGTCGGGG |
| | GGC |
| 620 | TGGTGGTGGTGGT |
| 621 | GGGACAGGGCAGGGACAGGG |
| 622 | GGGTCCGGGTCTGGGTCTGGG |
| 623 | GGGCAGGGTCTGGGCTGGG |
| 624 | GGGCTGGGATGGGAAAGGG |
| 625 | GGGCTCTGGGTGGGCCGGG |
| 626 | GGGCTGGGCTGGGCAGGG |
| 627 | GGGTGGGCTGGGAAGGG |
| 628 | GGGAGGGAGGGCTAGGG |
| 629 | GGGCAGGGCTGGGAGGG |
| 630 | GGGTGGGAGGGCTGGG |
| 631 | GCGTAACCTCCATCCGAGTTGCAAGAGAGGGAAACGCAGTCTC |
| 632 | GGAATTGCGGGAAAGGGGTCAACAGCCGTTCAGTACCAAGTCTCAGGG |
| | GAAACTTTGAGATGGCCTTGCAAAGGGTATGGTAATAAGCTGACGGAC |
| | ATGGTCCTAACCACGCAGCCAAGTCCTAAGTCAACAGATCTTCTGTTGA |
| | TATGGATGCAGTTCA |
| 633 | TTGACGCGGTTCTATCTAGTTACGCGTTAAACCAACTAGAAA |
| 634 | TTGACGCGGTTCTATCTACTTACGCGTTAAACCAACTAGAAA |
| 635 | CGCGAGTCTAGGGGATAACGCGTTAAACTTCCTAGAAGGCGGTT |
| 636 | CGCGGATCTAGATTGTAACGCGTTAAACCATCTAGAAGGCGGTT |
| 637 | CGCGTCGCTACCGCCCGGCGCGTTAAACACACTAGAAGGCGGTT |
| 638 | CGCGGTTCTATCTAGTTACGCGTTAAACCAACTAGAA |
| 639 | GGCCGGCATGGTCCCAGCCTCCTCGCTGGCGCCGGCTGGGCAACATGC |
| | TTCGGCATGGCGAATGGGAC |
| 640 | GGTGGGAGACGTCCCACC |
| 641 | TGGGAGACGTCCCACC |
| 642 | GAGCAGCATGGCGTCGCTGCTCAC |
| 643 | CCATCAGTTGACACCCTGAGG |
| 644 | GCAGACCTAAGTGGTGACATATGGTCTG |
| 645 | CATGCGATTAGAAATAATCGCATG |
| 646 | TCTGCCATCAAAGCTGCGACCGTGCTCAGTCTGGTGGGAGACGTCCCA |
| | CCGGCCGGCATGGTCCCAGCCTCCTCGCTGGCGCCGGCTGGGCAACAT |
| | GCTTCGGCATGGCGAATGGGAC |

In some embodiments, the secondary structure comprises a pseudoknot. In some embodiments, the secondary structure comprises a pseudoknot derived from a virus. In some embodiments, the secondary structure comprises a pseudoknot of a Moloney murine leukemia virus (M-MLV) genome (a mpknot). In some embodiments, the secondary structure comprises a nucleotide sequence selected from the group consisting of SEQ ID NOs: 612-632, or a nucleotide sequence having at least 80%, 85%, 90%, 95%, 96%, 97%, 98%, or 99% sequence identity therewith. In some embodiments, the secondary structure comprises a nucleotide sequence of SEQ ID NO: 619, or a nucleotide sequence having at least 80%, 85%, 90%, 95%, 96%, 97%, 98%, or 99% sequence identity therewith.

In some embodiments, the secondary structure comprises a quadruplex. In some embodiments, the secondary structure comprises a G-quadruplex.

In some embodiments, the secondary structure comprises a riboswitch aptamer. In some embodiments, the secondary structure comprises a riboswitch aptamer derived from a prequeosine-1 riboswitch aptamer. In some embodiments, the secondary structure comprises a modified prequeosine-1 riboswitch aptamer. In some embodiments, the secondary structure comprises a nucleotide sequence selected from the group consisting of SEQ ID NOs: 633-638, or a nucleotide sequence having at least 80%, 85%, 90%, 95%, 96%, 97%, 98%, or 99% sequence identity therewith. In some embodiments, the secondary structure comprises a nucleotide sequence of SEQ ID NO: 638, or a nucleotide sequence having at least 80%, 85%, 90%, 95%, 96%, 97%, 98%, or 99% sequence identity therewith.

In some embodiments, the secondary structure is linked to one or more other component of a PEgRNA via a linker. For example, in some embodiments, the secondary structure is at the 3′ end of the PERNA and is linked to the 3′ end of a PBS via a linker. In some embodiments, the secondary structure is at the 5′ end of the PEgRNA and is linked to the 5′ end of a spacer via a linker. In some embodiments, the linker is a nucleotide linker that is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the linker is 5 to 10 nucleotides in length. In some embodiments, the linker is 10 to 20 nucleotides in length. In some embodiments, the linker is 15 to 25 nucleotides in length. In some embodiments, the linker is 8 nucleotides in length.

In some embodiments, the linker is designed to minimize base pairing between the linker and another component of the PEgRNA. In some embodiments, the linker is designed to minimize base pairing between the linker and the spacer. In some embodiments, the linker is designed to minimize base pairing between the linker and the PBS. In some embodiments, the linker is designed to minimize base pairing between the linker and the editing template. In some embodiments, the linker is designed to minimize base pairing between the linker and the sequence of the RNA secondary structure. In some embodiments, the linker is optimized to minimize base pairing between the linker and another component of the PERNA, in order of the following priority: spacer, PBS, editing template and then scaffold. In some embodiments, base paring probability is calculated using ViennaRNA 2.0 under standard parameters (37° C., 1 M NaCl, 0.05 M MgCl2).

In some embodiments, the PEgRNA comprises a RNA secondary structure and/or a linker disclosed in Nelson et al. Engineered PEgRNAs improve prime editing efficiency. Nat Biotechnol. (2021), the entirety of which is incorporated herein by reference.

In some embodiments, a PEgRNA is transcribed from a nucleotide encoding the PEgRNA, for example, a DNA plasmid encoding the PEgRNA. In some embodiments, the PEgRNA comprises a self-cleaving element. In some embodiments, the self-cleaving element improves transcription and/or processing of the PEgRNA when transcribed form the nucleotide encoding the PEgRNA. In some embodiments, the PEgRNA comprises a hairpin or a RNA quadruplex. In some embodiments, the PEgRNA comprises a self-cleaving ribozyme element, for example, a hammerhead, a pistol, a hatchet, a hairpin, a VS, a twister, or a twister sister ribozyme. In some embodiments, the PEgRNA comprises a HDV ribozyme. In some embodiments, the PEgRNA comprises a hairpin recognized by Csy4. In some embodiments, the PEgRNA comprises an ENE motif. In some embodiments, the PEgRNA comprises an element for nuclear expression (ENE) from MALAT1 Inc RNA. In some embodiments, the PEgRNA comprises an ENE element from Kaposi's sarcoma-associated herpesvirus (KSHV). In some embodiments, the PEgRNA comprises a 3′ box of a U1 snRNA. In some embodiments, the PERNA forms a circular RNA.

In some embodiments, the PEgRNA comprises an RNA secondary structure or a motif that improves binding to the DNA-RNA duple or enhances PEgRNA activity. In some embodiments, the PEgRNA comprises a sequence derived from a native nucleotide element involved in reverse transcription, e.g., initiation of retroviral transcription. In some embodiments, the PERNA comprises a sequence of, or derived from, a primer binding site of a substrate of a reverse transcriptase, a polypurine tract (PPT), or a kissing loop. In some embodiments, the PEgRNA comprises a dimerization motif, a kissing loop, or a GNRA tetraloop-tetraloop receptor pair that results in circularization of the PEgRNA. In some embodiments, the PEgRNA comprises an RNA secondary structure of a motif that results in physical separation of the spacer and the PBS of the PEgRNA, thereby prevents occlusion of the spacer and improves PEgRNA activity. In some embodiments, the PEgRNA comprises a secondary structure or motif, e.g., a 5′ or 3′ extension in the spacer region that form a toehold or hairpin, wherein the secondary structure or motif competes favorably against annealing between the spacer and the PBS of the PERNA, thereby prevents occlusion of the spacer and improves PERNA activity.

Exemplary sequences for the first PEgRNA and the second PEgRNA are provided in Tables 9-26.

In some embodiments, a PEgRNA may comprise one or more additional nucleotides. For example, in some embodiments, a PEgRNA is produced by transcription from a template nucleotide, for example, a template plasmid. In some embodiments, a polynucleotide encoding the PEgRNA is appended with one or more additional nucleotides that improves PEgRNA function or expression, e.g., expression from a plasmid that encodes the PEgRNA. In some embodiments, a polynucleotide encoding a PERNA is appended with one or more additional nucleotides at the 5′ end or at the 3′ end. In some embodiments, the polynucleotide encoding the PEgRNA is appended with a guanine at the 5′ end, for example, if the first nucleotide at the 5′ end of the spacer is not a guanine. In some embodiments, a polynucleotide encoding the PEgRNA is appended with nucleotide sequence CACC at the 5′ end. In some embodiments, the polynucleotide encoding the PEgRNA is appended with an additional nucleotide adenine at the 3′ end, for example, if the last nucleotide at the 3′ end of the PBS is a Thymine. In some embodiments, the polynucleotide encoding the PEgRNA is appended with a PEgRNA comprises additional nucleotide sequence TTTTTT, TTTTTTT, TTTTT, or TTTT at the 3′ end. In some embodiments, the PEgRNA comprises the appended nucleotides from the transcription template. Accordingly, in some embodiments, the PEgRNA further comprises one or more nucleotides at the 5′ end or the 3′ end in addition to spacer, PBS, and RTT sequences. In some embodiments, the PEgRNA further comprises a guanine at the 5′ end, for example, when the first nucleotide at the 5′ end of the spacer is not a guanine. In some embodiments, the PEgRNA further comprises nucleotide sequence CACC at the 5′ end. In some embodiments, the PEgRNA further comprises an adenine at the 3′ end, for example, if the last nucleotide at the 3′ end of the PBS is a thymine. In some embodiments, the PEgRNA further comprises nucleotide sequence UUUUUUU, UUUUUU, UUUUU, or UUUU at the 3′ end.

A PEgRNA may also comprise optional modifiers, e.g., 3′ end modifier region and/or an 5′ end modifier region. In some embodiments, a PEgRNA comprises at least one nucleotide that is not part of a spacer, a gRNA core, or an extension arm.

The optional sequence modifiers could be positioned within or between any of the other regions shown, and not limited to being located at the 3′ and 5′ ends. In certain embodiments, the PEgRNA comprises secondary RNA structure, such as, but not limited to, aptamers, hairpins, stem/loops, toeloops, and/or RNA-binding protein recruitment domains (e.g., the MS2 aptamer which recruits and binds to the MS2 coat protein (MS2cp)). In some embodiments, a PEgRNA comprises a short stretch of uracil at the 5′ end or the 3′ end. For example, in some embodiments, a PEgRNA comprising a 3′ extension arm comprises a “UUU” sequence at the 3′ end of the extension arm. In some embodiments, a PEgRNA comprises a toeloop sequence at the 3′ end. In some embodiments, the PERNA comprises a 3′ extension arm and a toeloop sequence at the 3′ end of the extension arm. In some embodiments, the PEgRNA comprises a 5′ extension arm and a toeloop sequence at the 5′ end of the extension arm. In some embodiments, the PEgRNA comprises a toeloop element having the sequence 5′-GAAANNNNN-3′, wherein N is any nucleobase. In some embodiments, the secondary RNA structure is positioned within the spacer. In some embodiments, the secondary structure is positioned within the extension arm. In some embodiments, the secondary structure is positioned within the gRNA core. In some embodiments, the secondary structure is positioned between the spacer and the gRNA core, between the gRNA core and the extension arm, or between the spacer and the extension arm. In some embodiments, the secondary structure is positioned between the PBS and the editing template. In some embodiments the secondary structure is positioned at the 3′ end or at the 5′ end of the PEgRNA. In some embodiments, the PEgRNA comprises a transcriptional termination signal at the 3′ end of the PEgRNA. In some embodiments, a PERNA comprises up to 50 nucleotides, up to 40 nucleotides, up to 30 nucleotides, up to 20 nucleotides, or up to 10 nucleotides of optional sequence modifiers at either or both of the 3′ and 5′ ends of the PEgRNA. In addition to secondary RNA structures, the PEgRNA may comprise a chemical linker or a poly(N) linker or tail, where “N” can be any nucleobase. In some embodiments, the chemical linker may function to prevent reverse transcription of the gRNA core.

A PEgRNA of this disclosure, in some embodiments, may include modified nucleotides, e.g., chemically modified DNA or RNA nucleobases, and may include one or more nucleobase analogs (e.g., modifications which might add functionality, such as temperature resilience). In some embodiments, PEgRNAs as described herein may be chemically modified. The phrase “chemical modifications,” as used herein, can include modifications which introduce chemistries which differ from those seen in naturally occurring DNA or RNAs, for example, covalent modifications such as the introduction of modified nucleotides, (e.g., nucleotide analogs, or the inclusion of pendant groups which are not naturally found in DNA or RNA molecules).

In some embodiments, the PEgRNAs provided in this disclosure may have undergone a chemical or biological modifications. Modifications may be made at any position within a PERNA, and may include modification to a nucleobase or to a phosphate backbone of the PEgRNA. In some embodiments, chemical modifications can be structure guided modifications. In some embodiments, a chemical modification is at the 5′ end and/or the 3′ end of a PEgRNA. In some embodiments, a chemical modification may be within the spacer, the extension arm, the editing template sequence, or the primer binding site of a PEgRNA. In some embodiments, a chemical modification may be within the spacer or the gRNA core of a PEgRNA. In some embodiments, a chemical modification may be within the 3′ most nucleotides of a PEgRNA. In some embodiments, a chemical modification may be within the 3′ most end of a PEgRNA. In some embodiments, a chemical modification may be within the 5′ most end of a PERNA. In some embodiments, a PEgRNA comprises 1, 2, 3, 4, 5, 6, 7, 8, 9, 10 or more chemically modified nucleotides at the 3′ end. In some embodiments, a PEgRNA comprises 1, 2, 3, 4, 5, 6, 7, 8, 9, 10 or more chemically modified nucleotides at the 5′ end. In some embodiments, a PEgRNA comprises 1, 2, 3, 4, 5 or more chemically modified nucleotides at the 3′ end. In some embodiments, a PEgRNA comprises 1, 2, 3, 4, 5 or more chemically modified nucleotides at the 5′ end. In some embodiments, a PEgRNA comprises 1, 2, 3 or more chemically modified nucleotides at the 3′ end. In some embodiments, a PEgRNA comprises 1, 2, 3 or more chemically modified nucleotides at the 5′ end.

In some embodiments, a PERNA comprises 1, 2, 3, 4, 5, 6, 7, 8, 9, 10 or more contiguous chemically modified nucleotides at the 3′ end. In some embodiments, a PEgRNA comprises 1, 2, 3, 4, 5, 6, 7, 8, 9, 10 or more contiguous chemically modified nucleotides at the 5′ end. In some embodiments, a PEgRNA comprises 1, 2, 3, 4, or 5 contiguous chemically modified nucleotides at the 3′ end. In some embodiments, a PEgRNA comprises 1, 2, 3, 4, or 5 contiguous chemically modified nucleotides at the 5′ end. In some embodiments, a PERNA comprises 1, 2, or 3 contiguous chemically modified nucleotides at the 3′ end. In some embodiments, a PERNA comprises 1, 2, or 3 contiguous chemically modified nucleotides at the 5′ end. In some embodiments, a PEgRNA comprises 3 contiguous chemically modified nucleotides at the 3′ end.

In some embodiments, a PEgRNA comprises 1, 2, 3, 4, 5, or more chemically modified nucleotides near the 3′ end. In some embodiments, a PEgRNA comprises 3 contiguous chemically modified nucleotides at the 3′ end. In some embodiments, a PERNA comprises 3 contiguous chemically modified nucleotides at the 5′ end. In some embodiments, a PEgRNA comprises 1, 2, 3, 4, 5, or more chemically modified nucleotides near the 3′ end. In some embodiments, a PEgRNA comprises 1, 2, 3, 4, 5, or more contiguous chemically modified nucleotides near the 3′ end. In some embodiments, a PEgRNA comprises 1, 2, 3, 4, 5, or more chemically modified nucleotides near the 3′ end, where the 3′ most nucleotide is not modified, and the 1, 2, 3, 4, 5, or more chemically modified nucleotides precede the 3′ most nucleotide in a 5′-to-3′ order. In some embodiments, a PEgRNA comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more chemically modified nucleotides near the 3′ end, where the 3′ most nucleotide is not modified, and the 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more chemically modified nucleotides precede the 3′ most nucleotide in a 5′-to-3′ order.

In some embodiments, a PEgRNA comprises one or more chemically modified nucleotides in the gRNA core. In some embodiments, the gRNA core of a PERNA may comprise one or more regions of a base paired lower stem, a base paired upper stem, where the lower stem and upper stem may be connected by a bulge comprising unpaired RNAs. The gRNA core may further comprise a nexus distal from the spacer. In some embodiments, the gRNA core comprises one or more chemically modified nucleotides in the lower stem, upper stem, and/or the hairpin regions. In some embodiments, all of the nucleotides in the lower stem, upper stem, and/or the hairpin regions are chemically modified.

A chemical modification to a PEgRNA can comprise a 2′-O-thionocarbamate-protected nucleoside phosphoramidite, a 2′-O-methyl (M), a 2′-O-methyl 3′phosphorothioate (MS), or a 2′-O-methyl 3′thioPACE (MSP), or any combination thereof. In some embodiments, a chemically modified PEgRNA can comprise a ‘-O-methyl (M) RNA, a 2’-O-methyl 3′phosphorothioate (MS) RNA, a 2′-O-methyl 3′thioPACE (MSP) RNA, a 2′-F RNA, a phosphorothioate bond modification, any other chemical modifications known in the art, or any combination thereof. A chemical modification may also include, for example, the incorporation of non-nucleotide linkages or modified nucleotides into the PERNA (e.g., modifications to one or both of the 3′ and 5′ ends of a guide RNA molecule). Such modifications can include the addition of bases to an RNA sequence, complexing the RNA with an agent (e.g., a protein or a complementary nucleic acid molecule), and inclusion of elements which change the structure of an RNA molecule (e.g., which form secondary structures). In some embodiments, a prime editing composition for editing a TRAC gene contains one PEgRNA. Exemplary PEgRNAs as well as corresponding components of the PERNAs are provided in Tables 9-25. In some embodiments, the PEgRNA comprises a sequence or a component thereof as provided in Tables 9-25, for example, a spacer sequence, a RTT sequence, and a PBS sequence from the same Table of Tables 9-25, or a PEgRNA sequence selected from any one of Tables 9-25. In some embodiments, the PEgRNA comprises a sequence or a component thereof as provided in Tables 9-25, and further comprises one or more contiguous nucleotides at the 5′ end of the RTT that are complementary with the edit strand of the TRAC gene. In some embodiments, the PEgRNA comprises a spacer sequence, a RTT sequence, and a PBS sequence from the same Table of Tables 9-25, at further comprises at least 4 contiguous nucleotides at the 5′ end of the RTT that are complementary with the edit strand of the TRAC gene. In some embodiments, the PEgRNA further comprises at least 5, 6, 7, 8, 9, 10, 11, 12, 13, 14, 15, 16, 17, 18, 19, or more nucleotides at the 5′ end of the RTT that are complementary with the edit strand of the TRAC gene. Although all the sequences provided in Tables 9-25 are RNA sequences, “T” is used instead of a “U” in the sequences for consistency with the ST.26 standard.

A prime editing composition having such PEgRNA may be used to introduce a Bxb1 RSS or a Pa01 sequence into the TRAC gene.

In some embodiments, a prime editing composition or system is a dual prime editing composition or system. A dual prime editing composition or system can comprise two PEgRNAs, each comprising a spacer, a gRNA core, and an extension arm comprising an editing template and a primer binding site (PBS). An editing template can also be referred to as an RTT. Exemplary dual prime editing PEgRNAs for editing the TRAC gene, as well as corresponding components of PEgRNAs, are provided in Tables 6-25. Each of Tables 9-25 provide spacer and PBS sequences designed for editing the exon1/intron1 junction of the TRAC gene. PEgRNA spacers and PBSs designed to target the exon1/intron1 junction of the TRAC gene can result in insertion of a recombinase recognition sequence (RSS) encoded by the RTT(s) of the PERNA or the PEgRNA pair, wherein the position of insertion is downstream of the endogenous TRAC promoter. When contacted with the recombinase and an exogenous DNA sequence comprising a donor sequence and a corresponding RSS, the donor sequence, e.g., a donor sequence encoding a CAR, can be integrated in the TRAC gene under the control of the endogenous TRAC gene promoter. Without being bound by theory, placing the donor sequence under the control of endogenous regulatory element, e.g., the TRAC promoter, may reduce tonic signaling, delay or avoid accelerated cell differentiation and exhaustion, and increases the therapeutic potency of engineered immune cells, e.g., T cells. Tables 9-12 provide exemplary spacer sequences and PBS sequences of a 5′ PERNA (also referred to as a first PEgRNA in a pair of dual prime editing PEgRNAs), as well as exemplary 5′ PEgRNAs that contain a spacer and a PBS provided in the same table. Tables 13-25 provide exemplary spacer sequences and PBS sequences of a 3′ PEgRNA (also referred to as a second PEgRNA in a pair of dual prime editing PERNAs), as well as exemplary 3′ PEgRNAs that contain a spacer and a PBS provided in the same table. Each of Tables 9-25 contains three columns. The first column provides the sequence number; the second column provides the sequence of the PEgRNA or component, labeled with a SEQ ID NO, as required by ST.26 standard. The third column contains a description of the sequence. Table 6 provides exemplary RTT sequences. Table 7 provides exemplary gRNA core sequences. Table 8 provides exemplary structural motif sequences.

5′ PEgRNAs and 3′ PERNAs exemplified in Tables 9-25 can be used in a dual prime editing composition or system with any prime editor containing a Cas9 protein capable of recognizing a NGG PAM sequence, wherein N is A, G, C, or T. A dual prime editing composition or system for editing the TRAC gene can comprise (A) a 5′ PERNA (can also be referred to as a first PEgRNA) and a (B) 3′ PEgRNA (can also be referred to as a second PEgRNA), wherein the first PEgRNA comprises (i) a first spacer that comprises at its 3′ end a 5′ spacer sequence provided in any one of Tables 9-12; (ii) a first gRNA core capable of binding to a Cas9 protein; and (iii) a first extension arm comprising a first editing template and a first primer binding site (PBS), wherein the first PBS comprises at its 5′ end any 5′ PBS sequence provided in the same table as the first spacer; wherein the second PEgRNA comprises (i) a second spacer that comprises at its 3′ end a 3′ spacer sequence provided in any one of Tables 13-25; (ii) a second gRNA core capable of binding to a Cas9 protein; and (iii) a second extension arm comprising a second editing template and a second PBS, wherein the second PBS comprises at its 5′ end any 3′ PBS sequence provided in the same table as the second spacer, and wherein (a) the first editing template comprises a region of complementarity to the second editing template; (b) the first editing template comprises nucleotides (p-12) to (p-3) of the second spacer, wherein p is the length of the second spacer, and the second editing template comprises nucleotides (q-12) to (q-3) of the first spacer, wherein q is the length of the first spacer, or (c) the first editing template comprises nucleotides (p-12) to (p-3) of the second spacer and a region of complementarity to the second editing template, and the second editing template comprises nucleotides (q-12) to (q-3) of the first spacer and a region of complementarity to the first editing template.

The 5′ PEgRNA spacers and the 3′ PERNA spacers exemplified in Tables 9-25 can be, for example, 17 to 22 nucleotides in length. Each spacer within a single table correspond to the same PAM sequence and nick site when used with a compatible Cas9 nickase. In some embodiments, the 5′ PEgRNA spacer and/or the 3′ PEgRNA spacer are 20 nucleotides in length. The PBS of the 5′ PEgRNA and the 3′ PEgRNA can be, for example, 5 to 19 nucleotides in length. In some embodiments, the PBS is 8 to 17 nucleotides in length. In some embodiments, the PBS is 8 to 16 nucleotides in length. In some embodiments, the PBS is 8 to 13 nucleotides in length. In some embodiments, the PBS is 8, 10, or 12 nucleotides in length.

In some embodiments, a dual prime editing composition or system can comprise a 5′ PEgRNA and a 3′ PERNA, wherein the 5′ PERNA comprises a spacer selected from any one of Tables 9-12 and a PBS selected from the same table as the 5′ spacer, the 3′ PEgRNA comprises a spacer selected from Tables 13-25 and a PBS selected from the same table as a 3′ spacer, and wherein the RTT of the 5′ PEgRNA (the 5′ RTT) comprise a region of complementarity to the RTT of the 3′ PEgRNA (the 3′ RTT). The 5′ PEgRNA is capable of complexing with a prime editor comprising a Cas9 nickase (e.g. a Cas9 having an inactivated HNH nuclease domain) to generate a first nick, and the 3′ PEgRNA is capable of complexing with a prime editor comprising a Cas9 nickase (e.g. a Cas9 having an inactivated HNH nuclease domain) to generate a second nick on the TRAC gene. Accordingly, contacting the target TRAC gene with the dual prime editing system can result in deletion of the sequence between the first nick and the second nick (the deleted region referred to as the IND) and insertion of the replacement duplex (RD) encoded by the 5′ RTT and the 3′ RTT. The 5′ RTT and the 3′ RTT can be completely complementary to each other throughout their entire length, or they can be partially complementary, e.g., the region of complementarity can be at the 5′ ends of the 3′ RTT and the 5′ RTT. The region of complementarity (also referred to herein as the overlapping duplex or the OD) between the 5′ RTT and the 3′ RTT can have various lengths and GC content. In some embodiments, the OD is about 15 to 38 base pairs in length. In some embodiments, the OD is 18 to 38 base pairs in length. In some embodiments, the OD is 10, 11, 12, 13, 14, 15, 16, 17, 18, 19, 20, 21, 22, 23, 24, 25, 26, 27, 28, 29, 30, 31, 32, 33, 34, 35, 36, 37, or 38 base pairs in length. In some embodiments, the OD is 20, 21, 22, 23, 24, 25, 26, 27 or more base pairs in length. In some embodiments, the OD has a GC content of at least about 27%. In some embodiments, the OD has a GC content of about 30% to about 85%. In some embodiments, the OD has a GC content of about 40% to about 70%. In some embodiments, the OD has a GC content of about 63% to about 70%. The RD can also have various lengths and GC content. In some embodiments, when the 5′ RTT and the 3′ RTT are completely complementary to each other throughout their entire length, the OD and the RD have the same length and GC content. In some embodiments, the OD and the RD have different lengths and GC content. In some embodiments, the ratio of OD/RD is at least about 20%. In some embodiments, the ratio of OD/RD is about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% or more. In some embodiments, the ratio of OR/RD is about 52% or more. In some embodiments, the ratio of OR/RD is about 53% or more. In some embodiments, the ratio of OR/RD is about 55% or more. In some embodiments, the OD and the RD have the same sequence and length.

Exemplary RTT pairs are provided in Table 6. Each pair of RTT #1 and RTT #2 that have the same RTT pairing number can be used as the basis of the RTTs in a PEgRNA pair, with either RTT #1 or RTT #2 serving as the basis for the 5′ RTT and the other as the basis for the 3′ RTT. Each RTT #1 in Table 6 has a region of complementarity to the RTT #2 in the same RTT pair. In some embodiments, each RTT #1 having the RTT pair number of 2 or 3 can be used with any RTT #2 having the RTT pair number of 2 or 3. In some embodiments, a pair of RTTs having the same length is selected. In some embodiments, a PEgRNA pair will comprise the full length of RTT #1 and RTT #2 from a single RTT pair and will form an OD along their complete lengths. In other embodiments, at least one of the PEgRNA pair will comprise less than the full-length sequence of the RTT #1 and/or RTT #2 from a single RTT pair. Such RTT pairs will form an OD only at the 5′ ends of the RTTs (corresponding to the 3′ ends of the newly synthesized strands). In some such embodiments, the first editing template comprises a 5′ fragment of an RTT listed in Table 6 and wherein the second editing template comprises a full length or 5′ fragment of the corresponding RTT pair and wherein at least 10 nucleotides at the 5′ end of the first and second editing templates have perfect reverse complementarity to each other. In other such embodiments, the second editing template comprises a 5′ fragment of an RTT listed in Table 6 and wherein the first editing template comprises a full length or 5′ fragment of the corresponding RTT pair and wherein at least 10 nucleotides at the 5′ end of the first and second editing templates have perfect reverse complementarity to each other. In some embodiments, the first editing template comprises a 5′ fragment of SEQ ID NO: 27, wherein the second editing template comprises a full length or 5′ fragment of SEQ ID NO: 107, wherein at least 10 nucleotides at the 5′ end of the first and second editing templates have perfect reverse complementarity to each other. In some embodiments, the second editing template comprises a 5′ fragment of SEQ ID NO: 27, wherein the first editing template comprises a full length or 5′ fragment of SEQ ID NO: 107, and wherein at least 10 nucleotides at the 5′ end of the first and second editing templates have perfect reverse complementarity to each other. In some embodiments, the first editing template comprises a 5′ fragment of SEQ ID NO: 107, wherein the second editing template comprises a full length or 5′ fragment of SEQ ID NO: 27, wherein at least 10 nucleotides at the 5′ end of the first and second editing templates have perfect reverse complementarity to each other. In some embodiments, the second editing template comprises a 5′ fragment of SEQ ID NO: 107, wherein the first editing template comprises a full length or 5′ fragment of SEQ ID NO: 27, and wherein at least 10 nucleotides at the 5′ end of the first and second editing templates have perfect reverse complementarity to each other. In any embodiments, at least 15, 20, 25, 30, 35 or more nucleotides at the 5′ end of the first and second editing templates can have perfect reverse complementarity to each other. In some embodiments, 10, 11, 12, 13, 14, 15, 16, 17, 18, 19, 20, 21, 22, 23, 24, 25, 26, 27, 28, 29, 30, 31, 32, 33, 34, 35, 36, 37 or more nucleotides at the 5′ end of the first and the second editing templates have perfect complementarity to each other. In some embodiments,20, 21, 22, 23, 24, 25, 26, 27 or more nucleotides at the 5′ end of the first and second editing templates can have perfect reverse complementarity to each other. In some embodiments, 20, 21, 22, 27 or more nucleotides at the 5′ end of the first and second editing templates can have perfect reverse complementarity to each other. The RTT pairing information (RTT1 or RTT2) and corresponding recombinase of PERNA sequences are indicated in the third column of Tables 9-25. For example, 5′ PERNA (Bxb1_38nt_RTT1) describes a 5′ PEgRNA sequence wherein the RTT has the sequence of Bxb1_38nt_RTT #1 as provided in Table 6.

The exemplary RTTs in Table 6 encode attB sequences or portions thereof that are recognized by Bxb1 recombinase or Pa01 recombinase. In some embodiments, the first RTT and the second RTT can also encode one or more of any other recombinase recognition sequences (RSSs) disclosed herein or known in the art. Exemplary RSS sequences are provided in Table 5.

RTTs designed to encode one or more RSS sequence can result in incorporation of the RSS sequence(s) in the target TRAC gene, which can be recognized by a corresponding recombinase. When contacted with the recombinase, the edited TRAC gene can undergo recombination with an exogenous DNA sequence that comprises a corresponding RSS sequence, e.g., attP sequence, that can be recognized by the same recombinase. In some embodiments, the exogenous DNA sequence comprises a donor sequence linked to the corresponding RSS sequence, and recombinase mediated recombination can result in integration of the donor sequence in the target TRAC gene. Exemplary

RSS sequence pairs, e.g., attB-attP pairs, are provided in Table 5, where the orthogonal pairing relationship between the attB and the corresponding attP sequences are indicated by their identifiers in the first column.

The exemplary RTTs provided in Table 6 encode attB sequences or fragments thereof. A PEgRNA or a dual prime editing PEgRNA pair that can comprise an RTT or an RTT pair exemplified in Table 6, or a fragment thereof, and can be used in a prime editing composition that further comprises an exogenous DNA sequence comprising a donor sequence and a corresponding attP sequence for integration of the donor sequence in TRAC. However, PEgRNA sequences that comprise RTT or RTT pairs that encode an attP sequence (or a fragment thereof) and the spacer and PBS sequences described herein are also contemplated. Such PEgRNA, or a pair of such PEgRNA, can be used in a prime editing composition that further comprises an exogenous DNA sequence comprising a donor sequence and a corresponding attB sequence for integration of the donor sequence in TRAC.

The donor sequence for integration into the TRAC gene can encode any desired sequence, e.g., a chimeric antigen receptor (CAR), a T cell receptor (TCR), a B cell receptor, a NK cell receptor, a cytokine receptor, a chemokine receptor, a cytokine, a chemokine, a signaling protein, an antigen binding domain, an antibody, a kill switch, a reporter protein, a dominant negative mutant of a immune cell signaling protein, or a functional fragment or variant thereof. Without being bound by theory, compared to other strategies for donor integration, e.g., CAR integration, in immune cells such as semi-random integration via lentivirus or transposons, prime editing-and-recombinase-mediated integration in the TRAC gene as described herein is targeted to specific positions in the TRAC gene and reduces the risk of unintended disruption or activation of non-target genes. Without being bound by theory, compared to other targeted integration strategies such as nuclease and homology directed repair (HDR), prime editing-and-recombinase-mediated integration in the TRAC gene as described herein is unlikely to involve double strand breaks (DSBs) that are exposed to cellular repair mechanisms, and can result in improved integration efficiency and/or reduced risk associated with DSBs, e.g., genome rearrangements, chromothripsis, or unintended gene disruption or activation such as p53 activation.

Recombination of the exogenous DNA sequence comprising an RSS at the prime edited TRAC gene comprising a corresponding RSS can result in integration of the donor sequence in the TRAC gene flanked by the recombination products of the RSS sequences. For example, in some embodiments, the PEgRNA or PEgRNA pairs install an attB sequence in the TRAC gene, and the exogenous DNA sequence comprises a donor sequence and a corresponding attP sequence. After recombinase mediated recombination, the TRAC gene comprises the following insert sequence from 5′ to 3′: attL-donor sequence-attR. In some embodiments, the PEgRNA or PEgRNA pairs install an attP sequence in the TRAC gene, and the exogenous DNA sequence comprises a donor sequence and a corresponding attB sequence. After recombinase mediated recombination, the TRAC gene comprises the following insert sequence from 5′ to 3′: attR-donor sequence-attL. Accordingly, provided herein are edited cells that comprise in the TRAC gene an insert sequence, wherein the insert sequence comprises (i) 5′-attL-donor sequence-attR-3′ or (ii) 5′-attR-donor sequence-attL-3′. In some embodiments, the edited cells, e.g., T cells provide herein comprise in the TRAC gene an insert sequence, wherein the insert sequence comprises 5′-attL-donor sequence-attR-3′.

Position of the insert sequence depends on the PERNA spacers designed for integration of the attB (or attP) sequence encoded by the RTTs. In some embodiments, the insert sequence in the edited cell is between the first nick site according to the first PEgRNA and the second nick site according to the second PEgRNA of a dual prime editing PEgRNA pair (i.e. the region corresponding to the IND according to the PEgRNA pair). In some embodiments, the insert sequence is immediately downstream of the first nick site. In some embodiments, the insert sequence is immediately upstream of the first nick site. Nick site locations according to exemplary PEgRNA spacers provided herein in human chromosome 14 are provided in Table 45 below. Unless otherwise specified, chromosome locations and coding sequence positions (e.g. c.xxxx indicates position xxxx in the coding sequence of a certain gene) are as set forth in Genome Reference Consortium Human Build 38 (GrCH38). Accordingly, a cell, e.g., a T cell edited with the PEgRNA and PEgRNA components provided in Tables 9-25 and contacted with (i) a recombinase and (ii) a donor sequence linked to an attP sequence may comprise in the TRAC gene an insert sequence between the nick position of any 5′ spacer in Table 45 and the nick position of any 3′ spacer in Table 45, depending on the 5′ spacer and the 3′ spacer used in the first PEgRNA and the second PEgRNA, wherein the insert sequence comprises 5′-attL-donor sequence-attR-3′. In some embodiments, the edited cell comprises the insert sequence between human chromosome 14 positions 22547458 and 22547533. In some embodiments, the edited cell comprises the insert sequence between human chromosome 14 positions 22547458 and 22547522. In some embodiments, the edited cell comprises the insert sequence between human chromosome 14 positions 22547458 and 22547529. In some embodiments, the edited cell comprises the insert sequence between human chromosome 14 positions 22547449 and 22547533. In some embodiments, the edited cell comprises the insert sequence between human chromosome 14 positions 22547449 and 22547522. In some embodiments, the edited cell comprises the insert sequence between human chromosome 14 positions 22547449 and 22547529.

Exemplary sequences of attB-attP sequence pairs are provided in Table 5. In some embodiments, the attB sequence is GGCTTGTCGACGACGGCGGTCTCCGTCGTCAGGATCAT (SEQ ID NO: 1187). In some embodiments, the attP sequence is GGTTTGTCTGGTCAACCACCGCGGTCTCAGTGGTGTACGGTACAAACC (SEQ ID NO: 1188). In some embodiments, the attL sequence is GGCTTGTCGACGACGGCGGTCTCAGTGGTGTACGGTACAAACC (SEQ ID NO: 1046). In some embodiments, the attR sequence is GGTTTGTCTGGTCAACCACCGCGGTCTCCGTCGTCAGGATCAT (SEQ ID NO: 1047). In some embodiments, the recombinase is a Bxb1 recombinase. In some embodiments, the Bxb1 recombinase comprises an amino acid sequence having at least 85%, 90%, 95%, 96%, 97%, 98%, 99%, or 100% identity to SEQ ID NO: 1131.

TABLE 45

| | 5′ nick site chromosome |
|---|---|
| 5′ spacer SEQ ID NO | position |
| 1,2, 3, 4, 5, 6 (Table 9 PEgRNAs) | 22547458 |
| 58, 59, 60, 61, 62, 63 (Table 10 PEgRNAs) | 22547457 |
| 85. 86, 87, 88, 89, 90 (Table 11 PEgRNAs) | 22547449 |
| 147, 148, 149, 150, 151, 152 (Table 12 PEgRNAs) | 22547448 |
| | 3′ nick site chromosome |
| 3′ spacer SEQ ID NO | position |
| 174, 175, 176, 177, 178, 179 (Table 13 PEgRNAs) | 22547533 |
| 230, 231, 232, 233, 234, 235 (Table 14 PEgRNAs) | 22547523 |
| 257, 258, 259, 260, 261, 262 (Table 15 PEgRNAs) | 22547491 |
| 284, 285, 286, 287, 288, 289 (Table 16 PEgRNAs) | 22547528 |
| 311, 312, 313, 314, 315, 316 (Table 17 PEgRNAs) | 22547497 |
| 338, 339, 340, 341, 342, 343 (Table 18 PEgRNAs) | 22547579 |
| 365, 366, 367, 368, 369, 370 (Table 19 PEgRNAs) | 22547522 |
| 411, 412, 413, 414, 415, 416 (Table 20 PEgRNAs) | 22547485 |
| 438, 439, 440, 441, 442, 443 (Table 21 PERNAs) | 22547506 |
| 465, 466, 467, 468, 469, 470 (Table 22 PEgRNAs) | 22547560 |
| 492, 493, 494, 495, 496, 497 (Table 23 PEgRNAs) | 22547505 |
| 519, 520, 521, 522, 523, 524 (Table 24 PEgRNAs) | 22547529 |
| 563, 564, 565, 566, 567, 568 (Table 25 PERNAs) | 22547490 |

In some embodiments, a dual prime editing composition or system can comprise a 5′ PEgRNA and a 3′ PEgRNA, wherein the 5′ PEgRNA comprises a spacer selected from any one of Tables 9-12 and a PBS selected from the same table as the 5′ spacer, the 3′ PEgRNA comprises a spacer selected from Tables 13-25 and a PBS selected from the same table as a 3′ spacer, and wherein the 5′ RTT and the 3′ RTT comprises at its 3′ end nucleotides (q-12) to (q-3) of the 5′ spacer, wherein q is the length of the 5′ spacer. For example, when the 5′ spacer and the 3′ spacer both have a length of 20 nucleotides, the 5′ RTT comprises at its 3′ end nucleotides 8 to 17 of the 3′ spacer, and the 3′ RTT comprises at its 3′ end nucleotides 8 to 17 of the 5′ spacer. The 5′ PEgRNA is capable of complexing with a prime editor comprising a Cas9 nickase (e.g. a Cas9 having an inactivated HNH nuclease domain) to generate a first nick, and the 3′ PERNA is capable of complexing with a prime editor comprising a Cas9 nickase (e.g. a Cas9 having an inactivated HNH nuclease domain) to generate a second nick in the TRAC gene. Accordingly, the 5′ RTT comprises a region of complementarity to the endogenous TRAC sequence directly downstream of the second nick (i.e. the endogenous TRAC sequence directly downstream of nucleotide 3 of the search target sequence of the 3′ PEgRNA), and the 3′ RTT comprises a region of complementarity to the endogenous TRAC sequence directly downstream of the first nick (i.e. the endogenous TRAC sequence directly downstream of nucleotide 3 of the search target sequence of the 5′ PEgRNA). Contacting the target TRAC gene with the dual prime editing system can result in deletion of the sequence between the first nick and the second nick (the deleted region referred to as the IND) without insertion of an exogenous sequence. In some embodiments, the region of complementarity of the 5′ RTT to the endogenous TRAC sequence is at least about 15 nucleotides in length. In some embodiments, the region of complementarity of the 5′ RTT to the endogenous TRAC sequence is at least about 20 nucleotides in length. In some embodiments, the region of complementarity of the 5′ RTT to the endogenous TRAC sequence is at about 20 to 30 nucleotides in length. In some embodiments, the region of complementarity of the 5′ RTT to the endogenous TRAC sequence is at about 20, about 25, or about 30 nucleotides in length. In some embodiments, the region of complementarity of the 3′ RTT to the endogenous TRAC sequence is at least about 15 nucleotides in length. In some embodiments, the region of complementarity of the 3′ RTT to the endogenous TRAC sequence is at least about 20 nucleotides in length. In some embodiments, the region of complementarity of the 3′ RTT to the endogenous TRAC sequence is at about 20 to 30 nucleotides in length. In some embodiments, the region of complementarity of the 3′ RTT to the endogenous TRAC sequence is at about 20, about 25, or about 30 nucleotides in length.

Alternatively, in some embodiments, a dual prime editing composition or system can comprise a 5′ PEgRNA and a 3′ PEgRNA, wherein the 5′ PEgRNA comprises a spacer selected from any one of Tables 9-12 and a PBS selected from the same table as the 5′ spacer, the 3′ PEgRNA comprises a spacer selected from Tables 13-25 and a PBS selected from the same table as a 3′ spacer, and wherein the RTT of the 5′ PERNA (the 5′ RTT) comprise a region of complementarity to the RTT of the 3′ PEgRNA (the 3′ RTT) and further comprises nucleotides (p-12) to (p-3) of the 3′ spacer, wherein p is the length of the 3′ spacer, and wherein the 3′ RTT comprises a region of complementarity to the 5′ RTT and further comprises nucleotides (q-12) to (q-3) of the 5′ spacer, wherein q is the length of the 5′ spacer. Contacting the target TRAC gene with the dual prime editing system can result in deletion of the sequence between the first nick and the second nick (the deleted region referred to as the IND) and insertion of the overlapping duplex (OD) encoded by the 5′ RTT and the 3′ RTT.

The 5′ PEgRNA and the 3′ PERNA exemplified in Tables 9-25 can comprise, from 5′ to 3′, the spacer, the gRNA core, the editing template (RTT), and the PBS. The 3′ end of the RTT can be contiguous with the 5′ end of the PBS. The PEgRNA can comprise multiple RNA molecules or can be a single RNA molecule. Any PEgRNA exemplified in Tables 9-25 may comprise, or further comprise, a structural motif at the 5′ end of the PEgRNA and/or the 3′ end of the extension arm. In some embodiments, a structural motif that is capable of forming a secondary structure on its own, such as a hairpin, a pseudoknot, or other RNA structure or motif is used. In some embodiments, the 3′ motif is connected to the 3′ end of the PBS via a linker sequence. Exemplary structural motifs are found in Table 8. In some embodiments, the 3′ motif comprises a sequence of 3 to 6 Uracil, e.g., the sequence of UUUU. Without being bound by theory, such 3′ motifs are believed to increase PEgRNA stability. PEgRNA sequences exemplified in Tables 9-25 may also be adapted for expression from a nucleic acid template with a U6 promoter, for example, by including a 5′ terminal G if the spacer of the PEgRNA begins with another nucleotide and/or by including 6 or 7 T nucleotides at the 3′ end of the extension arm in the expression cassette. Those of skill in the art will recognize that the transcribed PEgRNA will contain a variable number of 3′ U nucleotides (e.g., 3-7 Us). The modifications included in the selection of full length 5′ PEgRNAs included in Tables 9-25 are annotated in column 3 (Description) of the tables.

The gRNA core sequence of the 5′ PEgRNA and the gRNA core of the 3′ PEgRNA can comprise a gRNA core capable of binding to a Cas9 nuclease. Exemplary gRNA core sequences are found in Table 7. In some embodiments, a PEgRNA comprises a gRNA core having SEQ ID NO: 590. In some embodiments, the PEgRNA comprises a gRNA core having SEQ ID NO: 604.

Any 5′ PEgRNA and 3′ PERNA sequence provided in Tables 9-25 may be chemically synthesized and may alternatively or additionally comprise one or more chemical modifications, such as phosphorothioate (PS) bond(s), 2′-O-methylated (2′-Ome) nucleotides, or a combination thereof. In some embodiments, the 5′ PEgRNA and/or the 3′ PEgRNA comprises 3′ mN*mN*mN*N and 5′mN*mN*mN* modifications, where m indicates that the nucleotide contains a 2′-O-Me modification and a * indicates the presence of a phosphorothioate bond. Any synthetically produced PEgRNA may additionally contain a series of four 3′ U nucleotides.

The PEgRNAs described herein can be used in combination in a Prime Editing composition or system with one or more additional PEgRNAs that are designed to edit a different location in the same target gene or to edit a different gene in human genome. For example, the PEgRNAs described herein can be used in a multiplexed Prime Editing system with one or more additional PEgRNAs that are designed to edit a different gene, for example, a B-2 microglobulin (B2M) gene that encodes the common subunit of HLA class-I antigen. Without being bound by theory, disruption of B2M gene can reduce or abolish expression of HLA class-I antigens on cell surface, resulting in HLA class-I-deficient cells, e.g., immune cells, that can evade attack by host cytotoxic T cells. Without being bound by theory, multiplexed disruption of MHC class I protein expression and T cell receptor expression in an engineered cell, e.g., immune cell, may reduce the allogenic reaction both of a host immune system and of the T cell receptor to host cells. Therefore, multiplexed disruption of T cell receptor gene(s), e.g., TRAC, and HLA class-I genes, e.g., B2M, may be used to generate engineered immune cells with reduced risk of graft versus host disease and graft rejection.

In some embodiments, a prime editing system, composition, or method of the disclosure targeting a TRAC locus further comprises a PEgRNA designed to edit a B2M gene or a nucleic acid encoding the PEgRNA as described herein.

Table 26 provides exemplary Prime Editing guide RNAs (PEgRNAs) and components designed to disrupt the B2M gene by introducing one or more premature in frame stop codons. The PEgRNAs in Table 26 can be used with any Prime Editor containing an appropriate Cas9 protein capable of recognizing an NGG PAM sequence, wherein N refers to any one of nucleotide A, G, C, or T. Exemplary Cas9 variants that can recognize an NGG PAM sequence are provided in Table 1C. Although all the sequences provided in Table 26 are RNA sequences, “T” is used instead of a “U” in the sequences for consistency with the ST.26 standard.

The B2M-editing PEgRNAs exemplified in Table 26 comprise: (a) a spacer comprising at its 3′ end a sequence corresponding to sequence number 1063; (b) a gRNA core capable of complexing with a Cas9 protein, and (c) an extension arm comprising: (i) an editing template comprising at its 3′ end (A) nucleotides 13-24 of sequence number 1079, (B) nucleotides 12-20 of sequence number 1085, or (C) nucleotides 7-17 of sequence number 1089, (ii) a prime binding site (PBS) comprising at its 5′ end a sequence corresponding to sequence number 1064.

The PEgRNA spacer can be, for example, 17-22 nucleotides in length. In some embodiments, the B2M-editing PEgRNA spacer is 17-20 nucleotides in length, and can comprise the sequence corresponding to any one of sequence numbers 1060, 1061, 1062, or 1063.

The editing template can be referred to as a reverse transcription template (RTT). The RTTs exemplified in Table 26 are designed to disrupt the B2M gene by introducing one or more in frame premature stop codons in the coding sequence of the B2M gene. For example, the editing template can encode an insertion of two consecutive stop codons or the complement thereof the two consecutive stop codons. In some embodiments, the insertion comprises a c.54insTAATAA edit. An exemplary RTT can comprise at its 3′ end the sequence corresponding to nucleotides 13-24, 12-24, 11-24, 10-24, 9-24, 8-24, 7-24, 6-24, 5-24, 4-24, 3-24, 2-24, or 1-24 of sequence number 1079. In some embodiments, the RTT comprises at its 3′ end the sequence corresponding to sequence number 1077, 1078, or 1079. Alternatively, the editing template can encode one or more frame shift mutations relative to the wildtype B2M gene. For example, the frame shift mutation can be a c.51delC deletion or the complement thereof. An exemplary RTT can comprise at its 3′ end the sequence corresponding to nucleotides 12-20, 11-20, 10-20, 9-20, 8-20, 7-20, 6-20, 5-20, 4-20, 3-20, 2-20, or 1-20 of sequence number 1085. In some embodiments, the RTT comprises at its 3′ end the sequence corresponding to sequence number 1080, 1081, 1082, 1083, 1084, or 1085. Alternatively, the frame shift mutation can be a c.50insG insertion or the complement thereof. An exemplary RTT can comprise at its 3′ end the sequence corresponding to nucleotides 7-17, 6-17, 5-17, 4-17, 3-17, 2-17, or 1-17 of sequence number 1089. In some embodiments, the RTT comprises at its 3′ end the sequence corresponding to sequence number 1086, 1087, 1088, or 1089. Nucleotide changes introduced by the edits encoded in the RTTs and PEgRNAs are indicated in the right column of Table 26. In some embodiments, the RTT comprises at least 4 contiguous nucleotides of complementarity with the edit strand, wherein the at least 4 contiguous nucleotides are located upstream of the 5′ most edit in the RTT.

The PBS of the B2M-editing PEgRNA can be, for example, 5 to 17 nucleotides in length and can comprise the sequence corresponding to any one of sequence numbers 1064-1076.

The B2M-editing PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule. Exemplary PERNAs provided in Table 26 can comprise a sequence corresponding to any one of sequence numbers 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, or 1120.

Any PEgRNA exemplified in Table 26 may comprise, or further comprise, a 3′ motif at the 3′ end of the extension arm, such as a universal motif, a sequence specific motif, or a series of 1, 2, 3, 4, 5, 6, 7 or more U nucleotides. In some embodiments, a universal or structural 3′ motif that is capable of forming a tertiary structure on its own such as a hairpin, a pseudoknot, or other RNA structure is used. In some embodiments, a sequence specific motif is used that is designed to hybridize with a portion of the RTT while not covering the PBS. Whether a universal or sequence specific motif is used, it can be connected to the 3′ of the PBS via a linker sequence. In some embodiments, the PEgRNA comprises 4 U nucleotides at its 3′ end. Without being bound by theory, such 3′ motifs are believed to increase PEgRNA stability. The PEgRNA may alternatively or additionally comprise one or more chemical modifications, such as phosphorothioate (PS) bond(s), 2′-O-methylated (2′-Ome) nucleotides, or a combination thereof. In some embodiments, the PEgRNA comprise 3′ mN*mN*mN*N and 5′mN*mN*mN* modifications, where m indicates that the nucleotide contains a 2′-O-Me modification and a * indicates the presence of a phosphorothioate bond. PEgRNA sequences exemplified in Table 26 may alternatively be adapted for expression from a nucleic acid template with a U6 promoter, for example, by including a 5′ terminal G if the spacer of the PEgRNA begins with another nucleotide, by including 6 or 7 U nucleotides at the 3′ end of the extension arm, or both. Such transcription-adapted sequences may further comprise a universal or sequence specific motif between the PBS and the 3′ terminal U series. The modifications included in the selection of full length PEgRNAs included in Table 26 are annotated in column 3 of Table 26.

Any of the B2M-editing PEgRNAs of Table 26 can be used in a Prime Editing system comprising any of the TRAC-editing PEgRNAs or TRAC-editing PEgRNA pairs provided herein. The Prime Editing system can further comprise a nick guide RNA (ngRNA) targeting the B2M gene. Without being bound by theory, a ngRNA can direct a Prime Editor to nick the non-edited strand (i.e. the non-PAM strand according to the PEgRNA), directing DNA repair to use the edited strand as a template, thereby increase editing efficiency. The B2M-editing ngRNA can comprise a spacer comprising at its 3′ end a sequence corresponding to nucleotides 4-20 of any ngRNA spacer listed in Table 26 and a gRNA core capable of complexing with a Cas9 protein. For example, the sequence in the spacer of the ngRNA can comprise nucleotides 4-20, 3-20, 2-20, or 1-20 of any one of sequence numbers 1121-1126. In some embodiments, the spacer of the B2M-editing ngRNA is a ngRNA spacer listed in Table 26. It can be advantageous to select a ngRNA spacer that has a PAM sequence compatible with the Cas9 protein used in the Prime Editor for the TRAC-editing PEgRNA or TRAC-editing PEgRNA pair as well as the B2M-editing PERNA, thus avoiding the need to use two or more different Cas9 proteins. The ngRNA is capable of directing a complexed Cas9 protein to bind the edit strand of the B2M gene; thus, a complexed Cas9 nickase containing a nuclease inactivating mutation in the HNH domain will nick the non-edit strand. A PE3 ngRNA spacer has perfect complementarity to the edit strand both pre- and post-edit; a PE3b ngRNA spacer has perfect complementarity to the edit strand post-edit. A B2M-editing PE3b ngRNA spacer in Table 26 annotated with the same * and number code as an RTT in Table 26 has perfect complementarity to the edit strand post-edit by a B2M-editing PEgRNA containing the RTT.

TABLE 9

| Sequence | | |
|---|---|---|
| Number | Sequence | Description |
| 1 | CTGGGTTGGGGCAAAGA (SEQ ID NO: 1) | 5′ PEgRNA spacer (GGG |
| | | PAM) |
| 2 | CCTGGGTTGGGGCAAAGA (SEQ ID NO: 2) | 5′ PEgRNA spacer (GGG |
| | | PAM) |
| 3 | GCCTGGGTTGGGGCAAAGA (SEQ ID NO: 3) | 5′ PEgRNA spacer (GGG |
| | | PAM) |
| 4 | AGCCTGGGTTGGGGCAAAGA (SEQ ID NO: 4) | 5′ PEgRNA spacer (GGG |
| | | PAM) |
| 5 | CAGCCTGGGTTGGGGCAAAGA (SEQ ID NO: 5) | 5′ PEgRNA spacer (GGG |
| | | PAM) |
| 6 | CCAGCCTGGGTTGGGGCAAAGA (SEQ ID NO: | 5′ PEgRNA spacer (GGG |
| | 6) | PAM) |
| 7 | TTGCC | 5′ PBS |
| 8 | TTGCCC | 5′ PBS |
| 9 | TTGCCCC | 5′ PBS |
| 10 | TTGCCCCA | 5′ PBS |
| 11 | TTGCCCCAA | 5′ PBS |
| 12 | TTGCCCCAAC (SEQ ID NO: 12) | 5′ PBS |
| 13 | TTGCCCCAACC (SEQ ID NO: 13) | 5′ PBS |
| 14 | TTGCCCCAACCC (SEQ ID NO: 14) | 5′ PBS |
| 15 | TTGCCCCAACCCA (SEQ ID NO: 15) | 5′ PBS |
| 16 | TTGCCCCAACCCAG (SEQ ID NO: 16) | 5′ PBS |
| 17 | TTGCCCCAACCCAGG (SEQ ID NO: 17) | 5′ PBS |
| 18 | TTGCCCCAACCCAGGC (SEQ ID NO: 18) | 5′ PBS |
| 19 | TTGCCCCAACCCAGGCT (SEQ ID NO: 19) | 5′ PBS |
| 20 | TTGCCCCAACCCAGGCTG (SEQ ID NO: 20) | 5′ PBS |
| 21 | TTGCCCCAACCCAGGCTGG (SEQ ID NO: 21) | 5′ PBS |
| 29 | AGCCTGGGTTGGGGCAAAGAGTTTTAGAGCT | 5′ pegRNA |
| | AGAAATAGCAAGTTAAAATAAGGCTAGTCCG | (Bxb1_29 nt_RTT1) |
| | TTATCAACTTGAAAAAGTGGCACCGAGTCGGT | |
| | GCACGACGGAGACCGCCGTCGTCGACAAGCC | |
| | TTGCCCC (SEQ ID NO: 29) | |
| 30 | AGCCTGGGTTGGGGCAAAGAGTTTTAGAGCT | 5′ pegRNA |
| | AGAAATAGCAAGTTAAAATAAGGCTAGTCCG | (Bxb1_29 nt_RTT1) |
| | TTATCAACTTGAAAAAGTGGCACCGAGTCGGT | |
| | GCACGACGGAGACCGCCGTCGTCGACAAGCC | |
| | TTGCCCCAACC (SEQ ID NO: 30) | |
| 31 | AGCCTGGGTTGGGGCAAAGAGTTTTAGAGCT | 5′ pegRNA |
| | AGAAATAGCAAGTTAAAATAAGGCTAGTCCG | (Pa01_1_27 nt_RTT1) |
| | TTATCAACTTGAAAAAGTGGCACCGAGTCGGT | |
| | GCTACGCCCTTCGAGCATGTAGGTCACGGTTG | |
| | CCCCAACCCA (SEQ ID NO: 31) | |
| 32 | AGCCTGGGTTGGGGCAAAGAGTTTTAGAGCT | 5′ pegRNA |
| | AGAAATAGCAAGTTAAAATAAGGCTAGTCCG | (Bxb1_30 nt_RTT1) |
| | TTATCAACTTGAAAAAGTGGCACCGAGTCGGT | |
| | GCGACGACGGAGACCGCCGTCGTCGACAAGC | |
| | CTTGCCCCAACC (SEQ ID NO: 32) | |
| 33 | AGCCTGGGTTGGGGCAAAGAGTTTTAGAGCT | 5′ pegRNA |
| | AGAAATAGCAAGTTAAAATAAGGCTAGTCCG | (Pa01_2_30 nt_RTT1) |
| | TTATCAACTTGAAAAAGTGGCACCGAGTCGGT | |
| | GCGCATACGCCCTTCGAGCATGTAGGTCACGG | |
| | TTGCCCCAACCCA (SEQ ID NO: 33) | |
| 34 | AGCCTGGGTTGGGGCAAAGAGTTTTAGAGCT | 5′ pegRNA |
| | AGAAATAGCAAGTTAAAATAAGGCTAGTCCG | (Bxb1_30 nt_RTT1) |
| | TTATCAACTTGAAAAAGTGGCACCGAGTCGGT | |
| | GCGACGACGGAGACCGCCGTCGTCGACAAGC | |
| | CTTGCCCCAACCCA (SEQ ID NO: 34) | |
| 35 | AGCCTGGGTTGGGGCAAAGAGTTTTAGAGCT | 5′ pegRNA |
| | AGAAATAGCAAGTTAAAATAAGGCTAGTCCG | (Pa01_1_27 nt_RTT1); |
| | TTATCAACTTGAAAAAGTGGCACCGAGTCGGT | contains 3′ end |
| | GCTACGCCCTTCGAGCATGTAGGTCACGGTTG | modification TTTT |
| | CCCCAACCCATTTT (SEQ ID NO: 35) | |
| 36 | AGCCTGGGTTGGGGCAAAGAGTTTTAGAGCT | 5′ pegRNA |
| | AGAAATAGCAAGTTAAAATAAGGCTAGTCCG | (Bxb1_30 nt_RTT1); contains |
| | TTATCAACTTGAAAAAGTGGCACCGAGTCGGT | 3′ end modification TTTT |
| | GCGACGACGGAGACCGCCGTCGTCGACAAGC | |
| | CTTGCCCCAACCTTTT (SEQ ID NO: 36) | |
| 37 | AGCCTGGGTTGGGGCAAAGAGTTTTAGAGCT | 5′ pegRNA |
| | AGAAATAGCAAGTTAAAATAAGGCTAGTCCG | (Bxb1_38 nt_RTT1) |
| | TTATCAACTTGAAAAAGTGGCACCGAGTCGGT | |
| | GCATGATCCTGACGACGGAGACCGCCGTCGT | |
| | CGACAAGCCTTGCCCC (SEQ ID NO: 37) | |
| 38 | AGCCTGGGTTGGGGCAAAGAGTTTTAGAGCT | 5′ pegRNA |
| | AGAAATAGCAAGTTAAAATAAGGCTAGTCCG | (Bxb1_30 nt_RTT1) |
| | TTATCAACTTGAAAAAGTGGCACCGAGTCGGT | |
| | GCGACGACGGAGACCGCCGTCGTCGACAAGC | |
| | CTTGCCCCAACCCAGG (SEQ ID NO: 38) | |
| 39 | AGCCTGGGTTGGGGCAAAGAGTTTTAGAGCT | 5′ pegRNA |
| | AGAAATAGCAAGTTAAAATAAGGCTAGTCCG | (Pa01_1_33 nt_RTT1) |
| | TTATCAACTTGAAAAAGTGGCACCGAGTCGGT | |
| | GCGGCGCATACGCCCTTCGAGCATGTAGGTCA | |
| | CGGTTGCCCCAACCCA (SEQ ID NO: 39) | |
| 40 | AGCCTGGGTTGGGGCAAAGAGTTTTAGAGCT | 5′ pegRNA |
| | AGAAATAGCAAGTTAAAATAAGGCTAGTCCG | (Pa01_2_30 nt_RTT1); |
| | TTATCAACTTGAAAAAGTGGCACCGAGTCGGT | contains 3′ end modification |
| | GCGCATACGCCCTTCGAGCATGTAGGTCACGG | TTTT |
| | TTGCCCCAACCCATTTT (SEQ ID NO: 40) | |
| 41 | AGCCTGGGTTGGGGCAAAGAGTTTTAGAGCT | 5′ pegRNA |
| | AGAAATAGCAAGTTAAAATAAGGCTAGTCCG | (Bxb1_30 nt_RTT1); contains |
| | TTATCAACTTGAAAAAGTGGCACCGAGTCGGT | 3′ end modification TTTT |
| | GCGACGACGGAGACCGCCGTCGTCGACAAGC | |
| | CTTGCCCCAACCCATTTT (SEQ ID NO: 41) | |
| 42 | AGCCTGGGTTGGGGCAAAGAGTTTTAGAGCT | 5′ pegRNA |
| | AGAAATAGCAAGTTAAAATAAGGCTAGTCCG | (Bxb1_30 nt_RTT1) |
| | TTATCAACTTGAAAAAGTGGCACCGAGTCGGT | |
| | GCGACGACGGAGACCGCCGTCGTCGACAAGC | |
| | CTTGCCCCAACCCAGGCT (SEQ ID NO: 42) | |
| 43 | AGCCTGGGTTGGGGCAAAGAGTTTTAGAGCT | 5′ pegRNA |
| | AGAAATAGCAAGTTAAAATAAGGCTAGTCCG | (Bxb1_38 nt_RTT1) |
| | TTATCAACTTGAAAAAGTGGCACCGAGTCGGT | |
| | GCATGATCCTGACGACGGAGACCGCCGTCGT | |
| | CGACAAGCCTTGCCCCAA (SEQ ID NO: 43) | |
| 44 | AGCCTGGGTTGGGGCAAAGAGTTTTAGAGCT | 5′ pegRNA |
| | AGAAATAGCAAGTTAAAATAAGGCTAGTCCG | (Bxb1_38 nt_RTT1) |
| | TTATCAACTTGAAAAAGTGGCACCGAGTCGGT | |
| | GCATGATCCTGACGACGGAGACCGCCGTCGT | |
| | CGACAAGCCTTGCCCCAACC (SEQ ID NO: 44) | |
| 45 | AGCCTGGGTTGGGGCAAAGAGTTTTAGAGCT | 5′ pegRNA |
| | AGAAATAGCAAGTTAAAATAAGGCTAGTCCG | (Bxb1_30 nt_RTT1); contains |
| | TTATCAACTTGAAAAAGTGGCACCGAGTCGGT | 3′ end modification TTTT |
| | GCGACGACGGAGACCGCCGTCGTCGACAAGC | |
| | CTTGCCCCAACCCAGGTTTT (SEQ ID NO: 45) | |
| 46 | AGCCTGGGTTGGGGCAAAGAGTTTTAGAGCT | 5′ pegRNA |
| | AGAAATAGCAAGTTAAAATAAGGCTAGTCCG | (Pa01_1_33 nt_RTT1); |
| | TTATCAACTTGAAAAAGTGGCACCGAGTCGGT | contains 3′ end modification |
| | GCGGCGCATACGCCCTTCGAGCATGTAGGTCA | TTTT |
| | CGGTTGCCCCAACCCATTTT (SEQ ID NO: 46) | |
| 47 | AGCCTGGGTTGGGGCAAAGAGTTTTAGAGCT | 5′ pegRNA |
| | AGAAATAGCAAGTTAAAATAAGGCTAGTCCG | (Bxb1_30 nt_RTT1); contains |
| | TTATCAACTTGAAAAAGTGGCACCGAGTCGGT | 3′ end modification TTTT |
| | GCGACGACGGAGACCGCCGTCGTCGACAAGC | |
| | CTTGCCCCAACCCAGGCTTTTT (SEQ ID NO: | |
| | 47) | |
| 48 | AGCCTGGGTTGGGGCAAAGAGTTTTAGAGCT | 5′ pegRNA |
| | AGAAATAGCAAGTTAAAATAAGGCTAGTCCG | (Bxb1_38 nt_RTT1); contains |
| | TTATCAACTTGAAAAAGTGGCACCGAGTCGGT | 3′ end modification TTTT |
| | GCATGATCCTGACGACGGAGACCGCCGTCGT | |
| | CGACAAGCCTTGCCCCAATTTT (SEQ ID NO: | |
| | 48) | |
| 49 | AGCCTGGGTTGGGGCAAAGAGTTTTAGAGCT | 5′ pegRNA |
| | AGAAATAGCAAGTTAAAATAAGGCTAGTCCG | (Bxb1_38 nt_RTT1) |
| | TTATCAACTTGAAAAAGTGGCACCGAGTCGGT | |
| | GCATGATCCTGACGACGGAGACCGCCGTCGT | |
| | CGACAAGCCTTGCCCCAACCCA (SEQ ID NO: | |
| | 49) | |
| 50 | AGCCTGGGTTGGGGCAAAGAGTTTTAGAGCT | 5′ pegRNA |
| | AGAAATAGCAAGTTAAAATAAGGCTAGTCCG | (Pa01_2_39 nt_RTT1) |
| | TTATCAACTTGAAAAAGTGGCACCGAGTCGGT | |
| | GCCTTCGTGGCGCATACGCCCTTCGAGCATGT | |
| | AGGTCACGGTTGCCCCAACCCA (SEQ ID NO: | |
| | 50) | |
| 51 | AGCCTGGGTTGGGGCAAAGAGTTTTAGAGCT | 5′ pegRNA |
| | AGAAATAGCAAGTTAAAATAAGGCTAGTCCG | (Bxb1_38 nt_RTT1); contains |
| | TTATCAACTTGAAAAAGTGGCACCGAGTCGGT | 3′ end modification TTTT |
| | GCATGATCCTGACGACGGAGACCGCCGTCGT | |
| | CGACAAGCCTTGCCCCAACCTTTT (SEQ ID NO: | |
| | 51) | |
| 52 | AGCCTGGGTTGGGGCAAAGAGTTTTAGAGCT | 5′ pegRNA |
| | AGAAATAGCAAGTTAAAATAAGGCTAGTCCG | (Bxb1_38 nt_RTT1) |
| | TTATCAACTTGAAAAAGTGGCACCGAGTCGGT | |
| | GCATGATCCTGACGACGGAGACCGCCGTCGT | |
| | CGACAAGCCTTGCCCCAACCCAGG (SEQ ID | |
| | NO: 52) | |
| 53 | AGCCTGGGTTGGGGCAAAGAGTTTTAGAGCT | 5′ pegRNA |
| | AGAAATAGCAAGTTAAAATAAGGCTAGTCCG | (Bxb1_38 nt_RTT1); contains |
| | TTATCAACTTGAAAAAGTGGCACCGAGTCGGT | 3′ end modification TTTT |
| | GCATGATCCTGACGACGGAGACCGCCGTCGT | |
| | CGACAAGCCTTGCCCCAACCCATTTT (SEQ ID | |
| | NO: 53) | |
| 54 | AGCCTGGGTTGGGGCAAAGAGTTTTAGAGCT | 5′ pegRNA |
| | AGAAATAGCAAGTTAAAATAAGGCTAGTCCG | (Bxb1_38 nt_RTT1) |
| | TTATCAACTTGAAAAAGTGGCACCGAGTCGGT | |
| | GCATGATCCTGACGACGGAGACCGCCGTCGT | |
| | CGACAAGCCTTGCCCCAACCCAGGCT (SEQ ID | |
| | NO: 54) | |
| 55 | AGCCTGGGTTGGGGCAAAGAGTTTTAGAGCT | 5′ pegRNA |
| | AGAAATAGCAAGTTAAAATAAGGCTAGTCCG | (Pa01_2_39 nt_RTT1); |
| | TTATCAACTTGAAAAAGTGGCACCGAGTCGGT | contains 3′ end modification |
| | GCCTTCGTGGCGCATACGCCCTTCGAGCATGT | TTTT |
| | AGGTCACGGTTGCCCCAACCCATTTT (SEQ ID | |
| | NO: 55) | |
| 56 | AGCCTGGGTTGGGGCAAAGAGTTTTAGAGCT | 5′ pegRNA |
| | AGAAATAGCAAGTTAAAATAAGGCTAGTCCG | (Bxb1_38 nt_RTT1); contains |
| | TTATCAACTTGAAAAAGTGGCACCGAGTCGGT | 3′ end modification TTTT |
| | GCATGATCCTGACGACGGAGACCGCCGTCGT | |
| | CGACAAGCCTTGCCCCAACCCAGGTTTT (SEQ | |
| | ID NO: 56) | |
| 57 | AGCCTGGGTTGGGGCAAAGAGTTTTAGAGCT | 5′ pegRNA |
| | AGAAATAGCAAGTTAAAATAAGGCTAGTCCG | (Bxb1_38 nt_RTT1); contains |
| | TTATCAACTTGAAAAAGTGGCACCGAGTCGGT | 3′ end modification TTTT |
| | GCATGATCCTGACGACGGAGACCGCCGTCGT | |
| | CGACAAGCCTTGCCCCAACCCAGGCTTTTT | |
| | (SEQ ID NO: 57) | |

TABLE 10

| Sequence | | |
|---|---|---|
| Number | Sequence | Description |
| 58 | CCTGGGTTGGGGCAAAG (SEQ ID NO: 58) | 5′ PERNA spacer (AGG |
| | | PAM) |
| 59 | GCCTGGGTTGGGGCAAAG (SEQ ID NO: 59) | 5′ PEgRNA spacer (AGG |
| | | PAM) |
| 60 | AGCCTGGGTTGGGGCAAAG (SEQ ID NO: 60) | 5′ PEgRNA spacer (AGG |
| | | PAM) |
| 61 | CAGCCTGGGTTGGGGCAAAG (SEQ ID NO: 61) | 5′ PEgRNA spacer (AGG |
| | | PAM) |
| 62 | CCAGCCTGGGTTGGGGCAAAG (SEQ ID NO: 62) | 5′ PEgRNA spacer (AGG |
| | | PAM) |
| 63 | TCCAGCCTGGGTTGGGGCAAAG (SEQ ID NO: | 5′ PEgRNA spacer (AGG |
| | 63) | PAM) |
| 64 | TGCCC | 5′ PBS |
| 65 | TGCCCC | 5′ PBS |
| 66 | TGCCCCA | 5′ PBS |
| 67 | TGCCCCAA | 5′ PBS |
| 68 | TGCCCCAAC | 5′ PBS |
| 69 | TGCCCCAACC (SEQ ID NO: 69) | 5′ PBS |
| 70 | TGCCCCAACCC (SEQ ID NO: 70) | 5′ PBS |
| 71 | TGCCCCAACCCA (SEQ ID NO: 71) | 5′ PBS |
| 72 | TGCCCCAACCCAG (SEQ ID NO: 72) | 5′ PBS |
| 73 | TGCCCCAACCCAGG (SEQ ID NO: 73) | 5′ PBS |
| 74 | TGCCCCAACCCAGGC (SEQ ID NO: 74) | 5′ PBS |
| 75 | TGCCCCAACCCAGGCT (SEQ ID NO: 75) | 5′ PBS |
| 76 | TGCCCCAACCCAGGCTG (SEQ ID NO: 76) | 5′ PBS |
| 77 | TGCCCCAACCCAGGCTGG (SEQ ID NO: 77) | 5′ PBS |
| 78 | TGCCCCAACCCAGGCTGGA (SEQ ID NO: 78) | 5′ PBS |
| 79 | CAGCCTGGGTTGGGGCAAAGGTTTTAGAGCT | 5′ pegRNA |
| | AGAAATAGCAAGTTAAAATAAGGCTAGTCCG | (Bxb1_38 nt_RTT1) |
| | TTATCAACTTGAAAAAGTGGCACCGAGTCGGT | |
| | GCATGATCCTGACGACGGAGACCGCCGTCGT | |
| | CGACAAGCCTGCCCCAA (SEQ ID NO: 79) | |
| 80 | CAGCCTGGGTTGGGGCAAAGGTTTTAGAGCT | 5′ pegRNA |
| | AGAAATAGCAAGTTAAAATAAGGCTAGTCCG | (Bxb1_38 nt_RTT1) |
| | TTATCAACTTGAAAAAGTGGCACCGAGTCGGT | |
| | GCATGATCCTGACGACGGAGACCGCCGTCGT | |
| | CGACAAGCCTGCCCCAACC (SEQ ID NO: 80) | |
| 81 | CAGCCTGGGTTGGGGCAAAGGTTTTAGAGCT | 5′ pegRNA |
| | AGAAATAGCAAGTTAAAATAAGGCTAGTCCG | (Bxb1_38 nt_RTT1); contains |
| | TTATCAACTTGAAAAAGTGGCACCGAGTCGGT | 3′ end modification TTTT |
| | GCATGATCCTGACGACGGAGACCGCCGTCGT | |
| | CGACAAGCCTGCCCCAATTTT (SEQ ID NO: 81) | |
| 82 | CAGCCTGGGTTGGGGCAAAGGTTTTAGAGCT | 5′ pegRNA |
| | AGAAATAGCAAGTTAAAATAAGGCTAGTCCG | (Bxb1_38 nt_RTT1) |
| | TTATCAACTTGAAAAAGTGGCACCGAGTCGGT | |
| | GCATGATCCTGACGACGGAGACCGCCGTCGT | |
| | CGACAAGCCTGCCCCAACCCA (SEQ ID NO: 82) | |
| 83 | CAGCCTGGGTTGGGGCAAAGGTTTTAGAGCT | 5′ pegRNA |
| | AGAAATAGCAAGTTAAAATAAGGCTAGTCCG | (Bxb1_38 nt_RTT1); contains |
| | TTATCAACTTGAAAAAGTGGCACCGAGTCGGT | 3′ end modification TTTT |
| | GCATGATCCTGACGACGGAGACCGCCGTCGT | |
| | CGACAAGCCTGCCCCAACCTTTT (SEQ ID NO: | |
| | 83) | |
| 84 | CAGCCTGGGTTGGGGCAAAGGTTTTAGAGCT | 5′ pegRNA |
| | AGAAATAGCAAGTTAAAATAAGGCTAGTCCG | (Bxb1_38 nt_RTT1); contains |
| | TTATCAACTTGAAAAAGTGGCACCGAGTCGGT | 3′ end modification TTTT |
| | GCATGATCCTGACGACGGAGACCGCCGTCGT | |
| | CGACAAGCCTGCCCCAACCCATTTT (SEQ ID | |
| | NO: 84) | |

TABLE 11

| Sequence | | |
|---|---|---|
| Number | Sequence | Description |
| 85 | GACTCCAGCCTGGGTTG (SEQ ID NO: 85) | 5′ PEgRNA spacer (GGG |
| | | PAM) |
| 86 | GGACTCCAGCCTGGGTTG (SEQ ID NO: 86) | 5′ PEgRNA spacer (GGG |
| | | PAM) |
| 87 | TGGACTCCAGCCTGGGTTG (SEQ ID NO: 87) | 5′ PEgRNA spacer (GGG |
| | | PAM) |
| 88 | CTGGACTCCAGCCTGGGTTG (SEQ ID NO: 88) | 5′ PERNA spacer (GGG |
| | | PAM) |
| 89 | TCTGGACTCCAGCCTGGGTTG (SEQ ID NO: 89) | 5′ PEgRNA spacer (GGG |
| | | PAM) |
| 90 | ATCTGGACTCCAGCCTGGGTTG (SEQ ID NO: | 5′ PEgRNA spacer (GGG |
| | 90) | PAM) |
| 91 | CCCAG | 5′ PBS |
| 92 | CCCAGG | 5′ PBS |
| 93 | CCCAGGC | 5′ PBS |
| 94 | CCCAGGCT | 5′ PBS |
| 95 | CCCAGGCTG | 5′ PBS |
| 96 | CCCAGGCTGG (SEQ ID NO: 96) | 5′ PBS |
| 97 | CCCAGGCTGGA (SEQ ID NO: 97) | 5′ PBS |
| 98 | CCCAGGCTGGAG (SEQ ID NO: 98) | 5′ PBS |
| 99 | CCCAGGCTGGAGT (SEQ ID NO: 99) | 5′ PBS |
| 100 | CCCAGGCTGGAGTC (SEQ ID NO: 100) | 5′ PBS |
| 101 | CCCAGGCTGGAGTCC (SEQ ID NO: 101) | 5′ PBS |
| 102 | CCCAGGCTGGAGTCCA (SEQ ID NO: 102) | 5′ PBS |
| 103 | CCCAGGCTGGAGTCCAG (SEQ ID NO: 103) | 5′ PBS |
| 104 | CCCAGGCTGGAGTCCAGA (SEQ ID NO: 104) | 5′ PBS |
| 105 | CCCAGGCTGGAGTCCAGAT (SEQ ID NO: 105) | 5′ PBS |
| 108 | CTGGACTCCAGCCTGGGTTGGTTTTAGAGCTA | 5′ pegRNA |
| | GAAATAGCAAGTTAAAATAAGGCTAGTCCGT | (Bxb1_29 nt_RTT1) |
| | TATCAACTTGAAAAAGTGGCACCGAGTCGGT | |
| | GCACGACGGAGACCGCCGTCGTCGACAAGCC | |
| | CCCAGG (SEQ ID NO: 108) | |
| 109 | CTGGACTCCAGCCTGGGTTGGTTTTAGAGCTA | 5′ pegRNA |
| | GAAATAGCAAGTTAAAATAAGGCTAGTCCGT | (Bxb1_29 nt_RTT1) |
| | TATCAACTTGAAAAAGTGGCACCGAGTCGGT | |
| | GCACGACGGAGACCGCCGTCGTCGACAAGCC | |
| | CCCAGGCTGG (SEQ ID NO: 109) | |
| 110 | CTGGACTCCAGCCTGGGTTGGTTTTAGAGCTA | 5′ pegRNA |
| | GAAATAGCAAGTTAAAATAAGGCTAGTCCGT | (Pa01_1_27 nt_RTT1) |
| | TATCAACTTGAAAAAGTGGCACCGAGTCGGT | |
| | GCTACGCCCTTCGAGCATGTAGGTCACGGCCC | |
| | AGGCTGGAGT (SEQ ID NO: 110) | |
| 111 | CTGGACTCCAGCCTGGGTTGGTTTTAGAGCTA | 5′ pegRNA |
| | GAAATAGCAAGTTAAAATAAGGCTAGTCCGT | (Bxb1_30 nt_RTT1) |
| | TATCAACTTGAAAAAGTGGCACCGAGTCGGT | |
| | GCGACGACGGAGACCGCCGTCGTCGACAAGC | |
| | CCCCAGGCTGGA (SEQ ID NO: 111) | |
| 112 | CTGGACTCCAGCCTGGGTTGGTTTTAGAGCTA | 5′ pegRNA |
| | GAAATAGCAAGTTAAAATAAGGCTAGTCCGT | (Bxb1_30 nt_RTT2) |
| | TATCAACTTGAAAAAGTGGCACCGAGTCGGT | |
| | GCGACGACGGCGGTCTCCGTCGTCAGGATCAT | |
| | CCCAGGCTGGA (SEQ ID NO: 112) | |
| 113 | CTGGACTCCAGCCTGGGTTGGTTTTAGAGCTA | 5′ pegRNA |
| | GAAATAGCAAGTTAAAATAAGGCTAGTCCGT | (Pa01_2_30 nt_RTT1) |
| | TATCAACTTGAAAAAGTGGCACCGAGTCGGT | |
| | GCGCATACGCCCTTCGAGCATGTAGGTCACGG | |
| | CCCAGGCTGGAGT (SEQ ID NO: 113) | |
| 114 | CTGGACTCCAGCCTGGGTTGGTTTTAGAGCTA | 5′ pegRNA |
| | GAAATAGCAAGTTAAAATAAGGCTAGTCCGT | (Bxb1_30 nt_RTT1) |
| | TATCAACTTGAAAAAGTGGCACCGAGTCGGT | |
| | GCGACGACGGAGACCGCCGTCGTCGACAAGC | |
| | CCCCAGGCTGGAGT (SEQ ID NO: 114) | |
| 115 | CTGGACTCCAGCCTGGGTTGGTTTTAGAGCTA | 5′ pegRNA |
| | GAAATAGCAAGTTAAAATAAGGCTAGTCCGT | (Bxb1_30 nt_RTT2) |
| | TATCAACTTGAAAAAGTGGCACCGAGTCGGT | |
| | GCGACGACGGCGGTCTCCGTCGTCAGGATCAT | |
| | CCCAGGCTGGAGT (SEQ ID NO: 115) | |
| 116 | CTGGACTCCAGCCTGGGTTGGTTTTAGAGCTA | 5′ pegRNA |
| | GAAATAGCAAGTTAAAATAAGGCTAGTCCGT | (Bxb1_38 nt_RTT1) |
| | TATCAACTTGAAAAAGTGGCACCGAGTCGGT | |
| | GCATGATCCTGACGACGGAGACCGCCGTCGT | |
| | CGACAAGCCCCCAGG (SEQ ID NO: 116) | |
| 594 | GCTGGACTCCAGCCTGGGTTGGTTTTAGAGCT | 5′ pegRNA |
| | AGAAATAGCAAGTTAAAATAAGGCTAGTCCG | (Pa01_1_27 nt_RTT1) |
| | TTATCAACTTGAAAAAGTGGCACCGAGTCGGT | |
| | GCTACGCCCTTCGAGCATGTAGGTCACGGCCC | |
| | AGGCTGGAGT (SEQ ID NO: 594) | |
| 117 | GCTGGACTCCAGCCTGGGTTGGTTTTAGAGCT | 5′ pegRNA |
| | AGAAATAGCAAGTTAAAATAAGGCTAGTCCG | (Pa01_1_27 nt_RTT1); |
| | TTATCAACTTGAAAAAGTGGCACCGAGTCGGT | contains 3′ end modification |
| | GCTACGCCCTTCGAGCATGTAGGTCACGGCCC | TTTT |
| | AGGCTGGAGTTTTT (SEQ ID NO: 117) | |
| 118 | CTGGACTCCAGCCTGGGTTGGTTTTAGAGCTA | 5′ pegRNA |
| | GAAATAGCAAGTTAAAATAAGGCTAGTCCGT | (Bxb1_30 nt_RTT1); contains |
| | TATCAACTTGAAAAAGTGGCACCGAGTCGGT | 3′ end modification TTTT |
| | GCGACGACGGAGACCGCCGTCGTCGACAAGC | |
| | CCCCAGGCTGGATTTT (SEQ ID NO: 118) | |
| 119 | CTGGACTCCAGCCTGGGTTGGTTTTAGAGCTA | 5′ pegRNA |
| | GAAATAGCAAGTTAAAATAAGGCTAGTCCGT | (Bxb1_30 nt_RTT2); contains |
| | TATCAACTTGAAAAAGTGGCACCGAGTCGGT | 3′ end modification TTTT |
| | GCGACGACGGCGGTCTCCGTCGTCAGGATCAT | |
| | CCCAGGCTGGATTTT (SEQ ID NO: 119) | |
| 120 | CTGGACTCCAGCCTGGGTTGGTTTTAGAGCTA | 5′ pegRNA |
| | GAAATAGCAAGTTAAAATAAGGCTAGTCCGT | (Bxb1_30 nt_RTT1) |
| | TATCAACTTGAAAAAGTGGCACCGAGTCGGT | |
| | GCGACGACGGAGACCGCCGTCGTCGACAAGC | |
| | CCCCAGGCTGGAGTCC (SEQ ID NO: 120) | |
| 121 | CTGGACTCCAGCCTGGGTTGGTTTTAGAGCTA | 5′ pegRNA |
| | GAAATAGCAAGTTAAAATAAGGCTAGTCCGT | (Pa01_1_33 nt_RTT1) |
| | TATCAACTTGAAAAAGTGGCACCGAGTCGGT | |
| | GCGGCGCATACGCCCTTCGAGCATGTAGGTCA | |
| | CGGCCCAGGCTGGAGT (SEQ ID NO: 121) | |
| 122 | CTGGACTCCAGCCTGGGTTGGTTTTAGAGCTA | 5′ pegRNA |
| | GAAATAGCAAGTTAAAATAAGGCTAGTCCGT | (Bxb1_38 nt_RTT1) |
| | TATCAACTTGAAAAAGTGGCACCGAGTCGGT | |
| | GCATGATCCTGACGACGGAGACCGCCGTCGT | |
| | CGACAAGCCCCCAGGCT (SEQ ID NO: 122) | |
| 123 | CTGGACTCCAGCCTGGGTTGGTTTTAGAGCTA | 5′ pegRNA |
| | GAAATAGCAAGTTAAAATAAGGCTAGTCCGT | (Bxb1_30 nt_RTT1); contains |
| | TATCAACTTGAAAAAGTGGCACCGAGTCGGT | 3′ end modification TTTT |
| | GCGACGACGGAGACCGCCGTCGTCGACAAGC | |
| | CCCCAGGCTGGAGTTTTT (SEQ ID NO: 123) | |
| 124 | CTGGACTCCAGCCTGGGTTGGTTTTAGAGCTA | 5′ pegRNA |
| | GAAATAGCAAGTTAAAATAAGGCTAGTCCGT | (Bxb1_30 nt_RTT2); contains |
| | TATCAACTTGAAAAAGTGGCACCGAGTCGGT | 3′ end modification TTTT |
| | GCGACGACGGCGGTCTCCGTCGTCAGGATCAT | |
| | CCCAGGCTGGAGTTTTT (SEQ ID NO: 124) | |
| 125 | CTGGACTCCAGCCTGGGTTGGTTTTAGAGCTA | 5′ pegRNA |
| | GAAATAGCAAGTTAAAATAAGGCTAGTCCGT | (Bxb1_30 nt_RTT1) |
| | TATCAACTTGAAAAAGTGGCACCGAGTCGGT | |
| | GCGACGACGGAGACCGCCGTCGTCGACAAGC | |
| | CCCCAGGCTGGAGTCCAG (SEQ ID NO: 125) | |
| 126 | CTGGACTCCAGCCTGGGTTGGTTTTAGAGCTA | 5′ pegRNA |
| | GAAATAGCAAGTTAAAATAAGGCTAGTCCGT | (Bxb1_38 nt_RTT1) |
| | TATCAACTTGAAAAAGTGGCACCGAGTCGGT | |
| | GCATGATCCTGACGACGGAGACCGCCGTCGT | |
| | CGACAAGCCCCCAGGCTGG (SEQ ID NO: 126) | |
| 592 | GCTGGACTCCAGCCTGGGTTGGTTTTAGAGCT | 5′ pegRNA |
| | AGAAATAGCAAGTTAAAATAAGGCTAGTCCG | (Pa01_2_30 nt_RTT1) |
| | TTATCAACTTGAAAAAGTGGCACCGAGTCGGT | |
| | GCGCATACGCCCTTCGAGCATGTAGGTCACGG | |
| | CCCAGGCTGGAGT (SEQ ID NO: 592) | |
| 127 | GCTGGACTCCAGCCTGGGTTGGTTTTAGAGCT | 5′ pegRNA |
| | AGAAATAGCAAGTTAAAATAAGGCTAGTCCG | (Pa01_2_30 nt_RTT1); |
| | TTATCAACTTGAAAAAGTGGCACCGAGTCGGT | contains 3′ end modification |
| | GCGCATACGCCCTTCGAGCATGTAGGTCACGG | TTTT |
| | CCCAGGCTGGAGTTTTT (SEQ ID NO: 127) | |
| 128 | CTGGACTCCAGCCTGGGTTGGTTTTAGAGCTA | 5′ pegRNA |
| | GAAATAGCAAGTTAAAATAAGGCTAGTCCGT | (Bxb1_30 nt_RTT1); contains |
| | TATCAACTTGAAAAAGTGGCACCGAGTCGGT | 3′ end modification TTTT |
| | GCGACGACGGAGACCGCCGTCGTCGACAAGC | |
| | CCCCAGGCTGGAGTCCTTTT (SEQ ID NO: 128) | |
| 129 | CTGGACTCCAGCCTGGGTTGGTTTTAGAGCTA | 5′ pegRNA |
| | GAAATAGCAAGTTAAAATAAGGCTAGTCCGT | (Bxb1_38 nt_RTT1) |
| | TATCAACTTGAAAAAGTGGCACCGAGTCGGT | |
| | GCATGATCCTGACGACGGAGACCGCCGTCGT | |
| | CGACAAGCCCCCAGGCTGGA (SEQ ID NO: 129) | |
| 130 | CTGGACTCCAGCCTGGGTTGGTTTTAGAGCTA | 5′ pegRNA |
| | GAAATAGCAAGTTAAAATAAGGCTAGTCCGT | (Bxb1_38 nt_RTT2) |
| | TATCAACTTGAAAAAGTGGCACCGAGTCGGT | |
| | GCGGCTTGTCGACGACGGCGGTCTCCGTCGTC | |
| | AGGATCATCCCAGGCTGGA (SEQ ID NO: 130) | |
| 131 | CTGGACTCCAGCCTGGGTTGGTTTTAGAGCTA | 5′ pegRNA |
| | GAAATAGCAAGTTAAAATAAGGCTAGTCCGT | (Bxb1_38 nt_RTT1); contains |
| | TATCAACTTGAAAAAGTGGCACCGAGTCGGT | 3′ end modification TTTT |
| | GCATGATCCTGACGACGGAGACCGCCGTCGT | |
| | CGACAAGCCCCCAGGCTTTTT (SEQ ID NO: | |
| | 131) | |
| 132 | GCTGGACTCCAGCCTGGGTTGGTTTTAGAGCT | 5′ pegRNA |
| | AGAAATAGCAAGTTAAAATAAGGCTAGTCCG | (Pa01_1_33 nt_RTT1); |
| | TTATCAACTTGAAAAAGTGGCACCGAGTCGGT | contains 3′ end modification |
| | GCGGCGCATACGCCCTTCGAGCATGTAGGTCA | TTTT |
| | CGGCCCAGGCTGGAGTTTTT (SEQ ID NO: 132) | |
| 133 | CTGGACTCCAGCCTGGGTTGGTTTTAGAGCTA | 5′ pegRNA |
| | GAAATAGCAAGTTAAAATAAGGCTAGTCCGT | (Bxb1_30 nt_RTT1); contains |
| | TATCAACTTGAAAAAGTGGCACCGAGTCGGT | 3′ end modification TTTT |
| | GCGACGACGGAGACCGCCGTCGTCGACAAGC | |
| | CCCCAGGCTGGAGTCCAGTTTT (SEQ ID NO: | |
| | 133) | |
| 134 | CTGGACTCCAGCCTGGGTTGGTTTTAGAGCTA | 5′ pegRNA |
| | GAAATAGCAAGTTAAAATAAGGCTAGTCCGT | (Bxb1_38 nt_RTT1) |
| | TATCAACTTGAAAAAGTGGCACCGAGTCGGT | |
| | GCATGATCCTGACGACGGAGACCGCCGTCGT | |
| | CGACAAGCCCCCAGGCTGGAGT (SEQ ID NO: | |
| | 134) | |
| 135 | CTGGACTCCAGCCTGGGTTGGTTTTAGAGCTA | 5′ pegRNA |
| | GAAATAGCAAGTTAAAATAAGGCTAGTCCGT | (Bxb1_38 nt_RTT2) |
| | TATCAACTTGAAAAAGTGGCACCGAGTCGGT | |
| | GCGGCTTGTCGACGACGGCGGTCTCCGTCGTC | |
| | AGGATCATCCCAGGCTGGAGT (SEQ ID NO: | |
| | 135) | |
| 136 | CTGGACTCCAGCCTGGGTTGGTTTTAGAGCTA | 5′ pegRNA |
| | GAAATAGCAAGTTAAAATAAGGCTAGTCCGT | (Bxb1_38 nt_RTT1); contains |
| | TATCAACTTGAAAAAGTGGCACCGAGTCGGT | 3′ end modification TTTT |
| | GCATGATCCTGACGACGGAGACCGCCGTCGT | |
| | CGACAAGCCCCCAGGCTGGTTTT (SEQ ID NO: | |
| | 136) | |
| 137 | CTGGACTCCAGCCTGGGTTGGTTTTAGAGCTA | 5′ pegRNA |
| | GAAATAGCAAGTTAAAATAAGGCTAGTCCGT | (Pa01_2_39 nt_RTT1) |
| | TATCAACTTGAAAAAGTGGCACCGAGTCGGT | |
| | GCCTTCGTGGCGCATACGCCCTTCGAGCATGT | |
| | AGGTCACGGCCCAGGCTGGAGT (SEQ ID NO: | |
| | 137) | |
| 138 | CTGGACTCCAGCCTGGGTTGGTTTTAGAGCTA | 5′ pegRNA |
| | GAAATAGCAAGTTAAAATAAGGCTAGTCCGT | (Bxb1_38 nt_RTT1); contains |
| | TATCAACTTGAAAAAGTGGCACCGAGTCGGT | 3′ end modification TTTT |
| | GCATGATCCTGACGACGGAGACCGCCGTCGT | |
| | CGACAAGCCCCCAGGCTGGATTTT (SEQ ID | |
| | NO: 138) | |
| 139 | CTGGACTCCAGCCTGGGTTGGTTTTAGAGCTA | 5′ pegRNA |
| | GAAATAGCAAGTTAAAATAAGGCTAGTCCGT | (Bxb1_38 nt_RTT2); contains |
| | TATCAACTTGAAAAAGTGGCACCGAGTCGGT | 3′ end modification TTTT |
| | GCGGCTTGTCGACGACGGCGGTCTCCGTCGTC | |
| | AGGATCATCCCAGGCTGGATTTT (SEQ ID NO: | |
| | 139) | |
| 140 | CTGGACTCCAGCCTGGGTTGGTTTTAGAGCTA | 5′ pegRNA |
| | GAAATAGCAAGTTAAAATAAGGCTAGTCCGT | (Bxb1_38 nt_RTT1) |
| | TATCAACTTGAAAAAGTGGCACCGAGTCGGT | |
| | GCATGATCCTGACGACGGAGACCGCCGTCGT | |
| | CGACAAGCCCCCAGGCTGGAGTCC (SEQ ID | |
| | NO: 140) | |
| 141 | CTGGACTCCAGCCTGGGTTGGTTTTAGAGCTA | 5′ pegRNA |
| | GAAATAGCAAGTTAAAATAAGGCTAGTCCGT | (Bxb1_38 nt_RTT1); contains |
| | TATCAACTTGAAAAAGTGGCACCGAGTCGGT | 3′ end modification TTTT |
| | GCATGATCCTGACGACGGAGACCGCCGTCGT | |
| | CGACAAGCCCCCAGGCTGGAGTTTTT (SEQ ID | |
| | NO: 141) | |
| 142 | CTGGACTCCAGCCTGGGTTGGTTTTAGAGCTA | 5′ pegRNA |
| | GAAATAGCAAGTTAAAATAAGGCTAGTCCGT | (Bxb1_38 nt_RTT2); contains |
| | TATCAACTTGAAAAAGTGGCACCGAGTCGGT | 3′ end modification TTTT |
| | GCGGCTTGTCGACGACGGCGGTCTCCGTCGTC | |
| | AGGATCATCCCAGGCTGGAGTTTTT (SEQ ID | |
| | NO: 142) | |
| 143 | CTGGACTCCAGCCTGGGTTGGTTTTAGAGCTA | 5′ pegRNA |
| | GAAATAGCAAGTTAAAATAAGGCTAGTCCGT | (Bxb1_38 nt_RTT1) |
| | TATCAACTTGAAAAAGTGGCACCGAGTCGGT | |
| | GCATGATCCTGACGACGGAGACCGCCGTCGT | |
| | CGACAAGCCCCCAGGCTGGAGTCCAG (SEQ ID | |
| | NO: 143) | |
| 593 | GCTGGACTCCAGCCTGGGTTGGTTTTAGAGCT | 5′ pegRNA |
| | AGAAATAGCAAGTTAAAATAAGGCTAGTCCG | (Pa01_2_39 nt_RTT1) |
| | TTATCAACTTGAAAAAGTGGCACCGAGTCGGT | |
| | GCCTTCGTGGCGCATACGCCCTTCGAGCATGT | |
| | AGGTCACGGCCCAGGCTGGAGT (SEQ ID NO: | |
| | 593) | |
| 144 | GCTGGACTCCAGCCTGGGTTGGTTTTAGAGCT | 5′ pegRNA |
| | AGAAATAGCAAGTTAAAATAAGGCTAGTCCG | (Pa01_2_39 nt_RTT1); |
| | TTATCAACTTGAAAAAGTGGCACCGAGTCGGT | contains 3′ end modification |
| | GCCTTCGTGGCGCATACGCCCTTCGAGCATGT | TTTT |
| | AGGTCACGGCCCAGGCTGGAGTTTTT (SEQ ID | |
| | NO: 144) | |
| 145 | CTGGACTCCAGCCTGGGTTGGTTTTAGAGCTA | 5′ pegRNA |
| | GAAATAGCAAGTTAAAATAAGGCTAGTCCGT | (Bxb1_38 nt_RTT1); contains |
| | TATCAACTTGAAAAAGTGGCACCGAGTCGGT | 3′ end modification TTTT |
| | GCATGATCCTGACGACGGAGACCGCCGTCGT | |
| | CGACAAGCCCCCAGGCTGGAGTCCTTTT (SEQ | |
| | ID NO: 145) | |
| 146 | CTGGACTCCAGCCTGGGTTGGTTTTAGAGCTA | 5′ pegRNA |
| | GAAATAGCAAGTTAAAATAAGGCTAGTCCGT | (Bxb1_38 nt_RTT1); contains |
| | TATCAACTTGAAAAAGTGGCACCGAGTCGGT | 3′ end modification TTTT |
| | GCATGATCCTGACGACGGAGACCGCCGTCGT | |
| | CGACAAGCCCCCAGGCTGGAGTCCAGTTTT | |
| | (SEQ ID NO: 146) | |
| 1132 | CTGGACTCCAGCCTGGGTTGGTTTTAGAGCTA | 5′ pegRNA |
| | GAAATAGCAAGTTAAAATAAGGCTAGTCCGT | (Bxb1_29 nt_RTT1); contains |
| | TATCAACTTGAAAAAGTGGCACCGAGTCGGT | 3′ end modification TTTT |
| | GCACGACGGAGACCGCCGTCGTCGACAAGCC | |
| | CCCAGGCTGGTTTT (SEQ ID NO: 1132) | |

TABLE 12

| Sequence | | |
|---|---|---|
| Number | Sequence | Description |
| 147 | GGACTCCAGCCTGGGTT (SEQ ID NO: 147) | 5′ PEgRNA spacer (GGG |
| | | PAM) |
| 148 | TGGACTCCAGCCTGGGTT (SEQ ID NO: 148) | 5′ PEgRNA spacer (GGG |
| | | PAM) |
| 149 | CTGGACTCCAGCCTGGGTT (SEQ ID NO: 149) | 5′ PEgRNA spacer (GGG |
| | | PAM) |
| 150 | TCTGGACTCCAGCCTGGGTT (SEQ ID NO: 150) | 5′ PERNA spacer (GGG |
| | | PAM) |
| 151 | ATCTGGACTCCAGCCTGGGTT (SEQ ID NO: | 5′ PEgRNA spacer (GGG |
| | 151) | PAM) |
| 152 | CATCTGGACTCCAGCCTGGGTT (SEQ ID NO: | 5′ PEgRNA spacer (GGG |
| | 152) | PAM) |
| 153 | CCAGG | 5′ PBS |
| 154 | CCAGGC | 5′ PBS |
| 155 | CCAGGCT | 5′ PBS |
| 156 | CCAGGCTG | 5′ PBS |
| 157 | CCAGGCTGG | 5′ PBS |
| 158 | CCAGGCTGGA (SEQ ID NO: 158) | 5′ PBS |
| 159 | CCAGGCTGGAG (SEQ ID NO: 159) | 5′ PBS |
| 160 | CCAGGCTGGAGT (SEQ ID NO: 160) | 5′ PBS |
| 161 | CCAGGCTGGAGTG (SEQ ID NO: 161) | 5′ PBS |
| 162 | CCAGGCTGGAGTGC (SEQ ID NO: 162) | 5′ PBS |
| 163 | CCAGGCTGGAGTGCA (SEQ ID NO: 163) | 5′ PBS |
| 164 | CCAGGCTGGAGTGCAA (SEQ ID NO: 164) | 5′ PBS |
| 165 | CCAGGCTGGAGTGCAAT (SEQ ID NO: 165) | 5′ PBS |
| 166 | CCAGGCTGGAGTGCAATG (SEQ ID NO: 166) | 5′ PBS |
| 167 | CCAGGCTGGAGTGCAATGG (SEQ ID NO: 167) | 5′ PBS |
| 168 | TCTGGACTCCAGCCTGGGTTGTTTTAGAGCTA | 5′ pegRNA |
| | GAAATAGCAAGTTAAAATAAGGCTAGTCCGT | (Bxb1_38 nt_RTT1) |
| | TATCAACTTGAAAAAGTGGCACCGAGTCGGT | |
| | GCATGATCCTGACGACGGAGACCGCCGTCGT | |
| | CGACAAGCCCCAGGCTG (SEQ ID NO: 168) | |
| 169 | TCTGGACTCCAGCCTGGGTTGTTTTAGAGCTA | 5′ pegRNA |
| | GAAATAGCAAGTTAAAATAAGGCTAGTCCGT | (Bxb1_38 nt_RTT1) |
| | TATCAACTTGAAAAAGTGGCACCGAGTCGGT | |
| | GCATGATCCTGACGACGGAGACCGCCGTCGT | |
| | CGACAAGCCCCAGGCTGGA (SEQ ID NO: 169) | |
| 170 | TCTGGACTCCAGCCTGGGTTGTTTTAGAGCTA | 5′ pegRNA |
| | GAAATAGCAAGTTAAAATAAGGCTAGTCCGT | (Bxb1_38 nt_RTT1); contains |
| | TATCAACTTGAAAAAGTGGCACCGAGTCGGT | 3′ end modification TTTT |
| | GCATGATCCTGACGACGGAGACCGCCGTCGT | |
| | CGACAAGCCCCAGGCTGTTTT (SEQ ID NO: | |
| | 170) | |
| 171 | TCTGGACTCCAGCCTGGGTTGTTTTAGAGCTA | 5′ pegRNA |
| | GAAATAGCAAGTTAAAATAAGGCTAGTCCGT | (Bxb1_38 nt_RTT1) |
| | TATCAACTTGAAAAAGTGGCACCGAGTCGGT | |
| | GCATGATCCTGACGACGGAGACCGCCGTCGT | |
| | CGACAAGCCCCAGGCTGGAGT (SEQ ID NO: | |
| | 171) | |
| 172 | TCTGGACTCCAGCCTGGGTTGTTTTAGAGCTA | 5′ pegRNA |
| | GAAATAGCAAGTTAAAATAAGGCTAGTCCGT | (Bxb1_38 nt_RTT1); contains |
| | TATCAACTTGAAAAAGTGGCACCGAGTCGGT | 3′ end modification TTTT |
| | GCATGATCCTGACGACGGAGACCGCCGTCGT | |
| | CGACAAGCCCCAGGCTGGATTTT (SEQ ID NO: | |
| | 172) | |
| 173 | TCTGGACTCCAGCCTGGGTTGTTTTAGAGCTA | 5′ pegRNA |
| | GAAATAGCAAGTTAAAATAAGGCTAGTCCGT | (Bxb1_38 nt_RTT1); contains |
| | TATCAACTTGAAAAAGTGGCACCGAGTCGGT | 3′ end modification TTTT |
| | GCATGATCCTGACGACGGAGACCGCCGTCGT | |
| | CGACAAGCCCCAGGCTGGAGTTTTT (SEQ ID | |
| | NO: 173) | |

TABLE 13

| Sequence | | |
|---|---|---|
| Number | Sequence | Description |
| 174 | GTCTCTCAGCTGGTACA (SEQ ID NO: 174) | 3′ PEgRNA spacer (CGG |
| | | PAM) |
| 175 | AGTCTCTCAGCTGGTACA (SEQ ID NO: 175) | 3′ PEgRNA spacer (CGG |
| | | PAM) |
| 176 | GAGTCTCTCAGCTGGTACA (SEQ ID NO: 176) | 3′ PEgRNA spacer (CGG |
| | | PAM) |
| 177 | AGAGTCTCTCAGCTGGTACA (SEQ ID NO: 177) | 3′ PEgRNA spacer (CGG |
| | | PAM) |
| 178 | TAGAGTCTCTCAGCTGGTACA (SEQ ID NO: | 3′ PEgRNA spacer (CGG |
| | 178) | PAM) |
| 179 | TTAGAGTCTCTCAGCTGGTACA (SEQ ID NO: | 3′ PEgRNA spacer (CGG |
| | 179) | PAM) |
| 180 | ACCAG | 3′ PBS |
| 181 | ACCAGC | 3′ PBS |
| 182 | ACCAGCT | 3′ PBS |
| 183 | ACCAGCTG | 3′ PBS |
| 184 | ACCAGCTGA | 3′ PBS |
| 185 | ACCAGCTGAG (SEQ ID NO: 185) | 3′ PBS |
| 186 | ACCAGCTGAGA (SEQ ID NO: 186) | 3′ PBS |
| 187 | ACCAGCTGAGAG (SEQ ID NO: 187) | 3′ PBS |
| 188 | ACCAGCTGAGAGA (SEQ ID NO: 188) | 3′ PBS |
| 189 | ACCAGCTGAGAGAC (SEQ ID NO: 189) | 3′ PBS |
| 190 | ACCAGCTGAGAGACT (SEQ ID NO: 190) | 3′ PBS |
| 191 | ACCAGCTGAGAGACTC (SEQ ID NO: 191) | 3′ PBS |
| 192 | ACCAGCTGAGAGACTCT (SEQ ID NO: 192) | 3′ PBS |
| 193 | ACCAGCTGAGAGACTCTA (SEQ ID NO: 193) | 3′ PBS |
| 194 | ACCAGCTGAGAGACTCTAA (SEQ ID NO: 194) | 3′ PBS |
| 200 | AGAGTCTCTCAGCTGGTACAGTTTTAGAGCTA | 3′ pegRNA |
| | GAAATAGCAAGTTAAAATAAGGCTAGTCCGT | (Bxb1_30 nt_RTT2) |
| | TATCAACTTGAAAAAGTGGCACCGAGTCGGT | |
| | GCACGGCGGTCTCCGTCGTCAGGATCATACCA | |
| | GCTGA (SEQ ID NO: 200) | |
| 201 | AGAGTCTCTCAGCTGGTACAGTTTTAGAGCTA | 3′ pegRNA |
| | GAAATAGCAAGTTAAAATAAGGCTAGTCCGT | (Pa01_1_27 nt_RTT2) |
| | TATCAACTTGAAAAAGTGGCACCGAGTCGGT | |
| | GCCCTACATGCTCGAAGGGCGTATGCGCCACC | |
| | AGCTGA (SEQ ID NO: 201) | |
| 202 | AGAGTCTCTCAGCTGGTACAGTTTTAGAGCTA | 3′ pegRNA |
| | GAAATAGCAAGTTAAAATAAGGCTAGTCCGT | (Bxb1_29 nt_RTT2) |
| | TATCAACTTGAAAAAGTGGCACCGAGTCGGT | |
| | GCACGACGGCGGTCTCCGTCGTCAGGATCATA | |
| | CCAGCTGA (SEQ ID NO: 202) | |
| 203 | AGAGTCTCTCAGCTGGTACAGTTTTAGAGCTA | 3′ pegRNA |
| | GAAATAGCAAGTTAAAATAAGGCTAGTCCGT | (Bxb1_30 nt_RTT2); contains |
| | TATCAACTTGAAAAAGTGGCACCGAGTCGGT | 3′ end modification TTTT |
| | GCACGGCGGTCTCCGTCGTCAGGATCATACCA | |
| | GCTGATTTT (SEQ ID NO: 203) | |
| 204 | AGAGTCTCTCAGCTGGTACAGTTTTAGAGCTA | 3′ pegRNA |
| | GAAATAGCAAGTTAAAATAAGGCTAGTCCGT | (Pa01_2_30 nt_RTT2) |
| | TATCAACTTGAAAAAGTGGCACCGAGTCGGT | |
| | GCACATGCTCGAAGGGCGTATGCGCCACGAA | |
| | GACCAGCTGA (SEQ ID NO: 204) | |
| 205 | AGAGTCTCTCAGCTGGTACAGTTTTAGAGCTA | 3′ pegRNA |
| | GAAATAGCAAGTTAAAATAAGGCTAGTCCGT | (Pa01_1_27 nt_RTT2); |
| | TATCAACTTGAAAAAGTGGCACCGAGTCGGT | contains 3′ end modification |
| | GCCCTACATGCTCGAAGGGCGTATGCGCCACC | TTTT |
| | AGCTGATTTT (SEQ ID NO: 205) | |
| 206 | AGAGTCTCTCAGCTGGTACAGTTTTAGAGCTA | 3′ pegRNA |
| | GAAATAGCAAGTTAAAATAAGGCTAGTCCGT | (Bxb1_30 nt_RTT2) |
| | TATCAACTTGAAAAAGTGGCACCGAGTCGGT | |
| | GCGACGACGGCGGTCTCCGTCGTCAGGATCAT | |
| | ACCAGCTGAGA (SEQ ID NO: 206) | |
| 207 | AGAGTCTCTCAGCTGGTACAGTTTTAGAGCTA | 3′ pegRNA |
| | GAAATAGCAAGTTAAAATAAGGCTAGTCCGT | (Bxb1_29 nt_RTT2) |
| | TATCAACTTGAAAAAGTGGCACCGAGTCGGT | |
| | GCACGACGGCGGTCTCCGTCGTCAGGATCATA | |
| | CCAGCTGAGAGA (SEQ ID NO: 207) | |
| 208 | AGAGTCTCTCAGCTGGTACAGTTTTAGAGCTA | 3′ pegRNA |
| | GAAATAGCAAGTTAAAATAAGGCTAGTCCGT | (Pa01_1_33 nt_RTT2) |
| | TATCAACTTGAAAAAGTGGCACCGAGTCGGT | |
| | GCCCGTGACCTACATGCTCGAAGGGCGTATGC | |
| | GCCACCAGCTGA (SEQ ID NO: 208) | |
| 209 | AGAGTCTCTCAGCTGGTACAGTTTTAGAGCTA | 3′ pegRNA |
| | GAAATAGCAAGTTAAAATAAGGCTAGTCCGT | (Pa01_2_30 nt_RTT2); |
| | TATCAACTTGAAAAAGTGGCACCGAGTCGGT | contains 3′ end modification |
| | GCACATGCTCGAAGGGCGTATGCGCCACGAA | TTTT |
| | GACCAGCTGATTTT (SEQ ID NO: 209) | |
| 210 | AGAGTCTCTCAGCTGGTACAGTTTTAGAGCTA | 3′ pegRNA |
| | GAAATAGCAAGTTAAAATAAGGCTAGTCCGT | (Bxb1_30 nt_RTT2) |
| | TATCAACTTGAAAAAGTGGCACCGAGTCGGT | |
| | GCGACGACGGCGGTCTCCGTCGTCAGGATCAT | |
| | ACCAGCTGAGAGA (SEQ ID NO: 210) | |
| 211 | AGAGTCTCTCAGCTGGTACAGTTTTAGAGCTA | 3′ pegRNA |
| | GAAATAGCAAGTTAAAATAAGGCTAGTCCGT | (Bxb1_30 nt_RTT1) |
| | TATCAACTTGAAAAAGTGGCACCGAGTCGGT | |
| | GCGACGACGGAGACCGCCGTCGTCGACAAGC | |
| | CACCAGCTGAGAGA (SEQ ID NO: 211) | |
| 212 | AGAGTCTCTCAGCTGGTACAGTTTTAGAGCTA | 3′ pegRNA |
| | GAAATAGCAAGTTAAAATAAGGCTAGTCCGT | (Bxb1_30 nt_RTT2); contains |
| | TATCAACTTGAAAAAGTGGCACCGAGTCGGT | 3′ end modification TTTT |
| | GCGACGACGGCGGTCTCCGTCGTCAGGATCAT | |
| | ACCAGCTGAGATTTT (SEQ ID NO: 212) | |
| 213 | AGAGTCTCTCAGCTGGTACAGTTTTAGAGCTA | 3′ pegRNA |
| | GAAATAGCAAGTTAAAATAAGGCTAGTCCGT | (Bxb1_30 nt_RTT2) |
| | TATCAACTTGAAAAAGTGGCACCGAGTCGGT | |
| | GCGACGACGGCGGTCTCCGTCGTCAGGATCAT | |
| | ACCAGCTGAGAGACT (SEQ ID NO: 213) | |
| 214 | AGAGTCTCTCAGCTGGTACAGTTTTAGAGCTA | 3′ pegRNA |
| | GAAATAGCAAGTTAAAATAAGGCTAGTCCGT | (Pa01_1_33 nt_RTT2); |
| | TATCAACTTGAAAAAGTGGCACCGAGTCGGT | contains 3′ end modification |
| | GCCCGTGACCTACATGCTCGAAGGGCGTATGC | TTTT |
| | GCCACCAGCTGATTTT (SEQ ID NO: 214) | |
| 215 | AGAGTCTCTCAGCTGGTACAGTTTTAGAGCTA | 3′ pegRNA |
| | GAAATAGCAAGTTAAAATAAGGCTAGTCCGT | (Bxb1_30 nt_RTT2); contains |
| | TATCAACTTGAAAAAGTGGCACCGAGTCGGT | 3′ end modification TTTT |
| | GCGACGACGGCGGTCTCCGTCGTCAGGATCAT | |
| | ACCAGCTGAGAGATTTT (SEQ ID NO: 215) | |
| 216 | AGAGTCTCTCAGCTGGTACAGTTTTAGAGCTA | 3′ pegRNA |
| | GAAATAGCAAGTTAAAATAAGGCTAGTCCGT | (Bxb1_30 nt_RTT1); contains |
| | TATCAACTTGAAAAAGTGGCACCGAGTCGGT | 3′ end modification TTTT |
| | GCGACGACGGAGACCGCCGTCGTCGACAAGC | |
| | CACCAGCTGAGAGATTTT (SEQ ID NO: 216) | |
| 217 | AGAGTCTCTCAGCTGGTACAGTTTTAGAGCTA | 3′ pegRNA |
| | GAAATAGCAAGTTAAAATAAGGCTAGTCCGT | (Bxb1_38 nt_RTT2) |
| | TATCAACTTGAAAAAGTGGCACCGAGTCGGT | |
| | GCGGCTTGTCGACGACGGCGGTCTCCGTCGTC | |
| | AGGATCATACCAGCTGA (SEQ ID NO: 217) | |
| 218 | AGAGTCTCTCAGCTGGTACAGTTTTAGAGCTA | 3′ pegRNA |
| | GAAATAGCAAGTTAAAATAAGGCTAGTCCGT | (Pa01_2_39 nt_RTT2) |
| | TATCAACTTGAAAAAGTGGCACCGAGTCGGT | |
| | GCCCGTGACCTACATGCTCGAAGGGCGTATGC | |
| | GCCACGAAGACCAGCTGA (SEQ ID NO: 218) | |
| 219 | AGAGTCTCTCAGCTGGTACAGTTTTAGAGCTA | 3′ pegRNA |
| | GAAATAGCAAGTTAAAATAAGGCTAGTCCGT | (Bxb1_30 nt_RTT2); contains |
| | TATCAACTTGAAAAAGTGGCACCGAGTCGGT | 3′ end modification TTTT |
| | GCGACGACGGCGGTCTCCGTCGTCAGGATCAT | |
| | ACCAGCTGAGAGACTTTTT (SEQ ID NO: 219) | |
| 220 | AGAGTCTCTCAGCTGGTACAGTTTTAGAGCTA | 3′ pegRNA |
| | GAAATAGCAAGTTAAAATAAGGCTAGTCCGT | (Bxb1_38 nt_RTT2) |
| | TATCAACTTGAAAAAGTGGCACCGAGTCGGT | |
| | GCGGCTTGTCGACGACGGCGGTCTCCGTCGTC | |
| | AGGATCATACCAGCTGAGA (SEQ ID NO: 220) | |
| 221 | AGAGTCTCTCAGCTGGTACAGTTTTAGAGCTA | 3′ pegRNA |
| | GAAATAGCAAGTTAAAATAAGGCTAGTCCGT | (Bxb1_38 nt_RTT2) |
| | TATCAACTTGAAAAAGTGGCACCGAGTCGGT | |
| | GCGGCTTGTCGACGACGGCGGTCTCCGTCGTC | |
| | AGGATCATACCAGCTGAGAGA (SEQ ID NO: | |
| | 221) | |
| 222 | AGAGTCTCTCAGCTGGTACAGTTTTAGAGCTA | 3′ pegRNA |
| | GAAATAGCAAGTTAAAATAAGGCTAGTCCGT | (Bxb1_38 nt_RTT2); contains |
| | TATCAACTTGAAAAAGTGGCACCGAGTCGGT | 3′ end modification TTTT |
| | GCGGCTTGTCGACGACGGCGGTCTCCGTCGTC | |
| | AGGATCATACCAGCTGATTTT (SEQ ID NO: | |
| | 222) | |
| 223 | AGAGTCTCTCAGCTGGTACAGTTTTAGAGCTA | 3′ pegRNA |
| | GAAATAGCAAGTTAAAATAAGGCTAGTCCGT | (Pa01_2_39 nt_RTT2); |
| | TATCAACTTGAAAAAGTGGCACCGAGTCGGT | contains 3′ end modification |
| | GCCCGTGACCTACATGCTCGAAGGGCGTATGC | TTTT |
| | GCCACGAAGACCAGCTGATTTT (SEQ ID NO: | |
| | 223) | |
| 224 | AGAGTCTCTCAGCTGGTACAGTTTTAGAGCTA | 3′ pegRNA |
| | GAAATAGCAAGTTAAAATAAGGCTAGTCCGT | (Bxb1_38 nt_RTT2); contains |
| | TATCAACTTGAAAAAGTGGCACCGAGTCGGT | 3′ end modification TTTT |
| | GCGGCTTGTCGACGACGGCGGTCTCCGTCGTC | |
| | AGGATCATACCAGCTGAGATTTT (SEQ ID NO: | |
| | 224) | |
| 225 | AGAGTCTCTCAGCTGGTACAGTTTTAGAGCTA | 3′ pegRNA |
| | GAAATAGCAAGTTAAAATAAGGCTAGTCCGT | (Bxb1_38 nt_RTT2) |
| | TATCAACTTGAAAAAGTGGCACCGAGTCGGT | |
| | GCGGCTTGTCGACGACGGCGGTCTCCGTCGTC | |
| | AGGATCATACCAGCTGAGAGACT (SEQ ID NO: | |
| | 225) | |
| 226 | AGAGTCTCTCAGCTGGTACAGTTTTAGAGCTA | 3′ pegRNA |
| | GAAATAGCAAGTTAAAATAAGGCTAGTCCGT | (Bxb1_38 nt_RTT2); contains |
| | TATCAACTTGAAAAAGTGGCACCGAGTCGGT | 3′ end modification TTTT |
| | GCGGCTTGTCGACGACGGCGGTCTCCGTCGTC | |
| | AGGATCATACCAGCTGAGAGATTTT (SEQ ID | |
| | NO: 226) | |
| 591 | AGAGTCTCTCAGCTGGTACAGTTTTAGAGCTA | 3′ pegRNA |
| | GAAATAGCAAGTTAAAATAAGGCTAGTCCGT | (Bxb1_38 nt_RTT1) |
| | TATCAACTTGAAAAAGTGGCACCGAGTCGGT | |
| | GCATGATCCTGACGACGGAGACCGCCGTCGT | |
| | CGACAAGCCACCAGCTGAGAGA (SEQ ID NO: | |
| | 591) | |
| 227 | AGAGTCTCTCAGCTGGTACAGTTTTAGAGCTA | 3′ pegRNA |
| | GAAATAGCAAGTTAAAATAAGGCTAGTCCGT | (Bxb1_38 nt_RTT1) |
| | TATCAACTTGAAAAAGTGGCACCGAGTCGGT | |
| | GCATGATCCTGACGACGGAGACCGCCGTCGT | |
| | CGACAAGCCACCAGCTGAGAGATTTT (SEQ ID | |
| | NO: 227) | |
| 228 | AGAGTCTCTCAGCTGGTACAGTTTTAGAGCTA | 3′ pegRNA |
| | GAAATAGCAAGTTAAAATAAGGCTAGTCCGT | (Bxb1_38 nt_RTT2); contains |
| | TATCAACTTGAAAAAGTGGCACCGAGTCGGT | 3′ end modification TTTT |
| | GCGGCTTGTCGACGACGGCGGTCTCCGTCGTC | |
| | AGGATCATACCAGCTGAGAGACTTTTT (SEQ | |
| | ID NO: 228) | |
| 229 | AGAGTCTCTCAGCTGGTACAGTTTTAGAGCTA | 3′ pegRNA |
| | GAAATAGCAAGTTAAAATAAGGCTAGTCCGT | (Bxb1_38 nt_RTT1); contains |
| | TATCAACTTGAAAAAGTGGCACCGAGTCGGT | 3′ end modification TTTT |
| | GCATGATCCTGACGACGGAGACCGCCGTCGT | |
| | CGACAAGCCACCAGCTGAGAGATTTTTTTT | |
| | (SEQ ID NO: 229) | |
| 595 | AGAGTCTCTCAGCTGGTACAGTTTTAGAGCTA | 3′ pegRNA |
| | GAAATAGCAAGTTAAAATAAGGCTAGTCCGT | (Pa01_2_30 nt_RTT2) |
| | TATCAACTTGAAAAAGTGGCACCGAGTCGGT | |
| | GCACATGCTCGAAGGGCGTATGCGCCACGAA | |
| | GACCAGCTGAGAGA (SEQ ID NO: 595) | |
| 596 | AGAGTCTCTCAGCTGGTACAGTTTTAGAGCTA | 3′ pegRNA |
| | GAAATAGCAAGTTAAAATAAGGCTAGTCCGT | (Pa01_2_30 nt_RTT2); |
| | TATCAACTTGAAAAAGTGGCACCGAGTCGGT | contains 3′ end modification |
| | GCACATGCTCGAAGGGCGTATGCGCCACGAA | TTTT |
| | GACCAGCTGAGAGATTTT (SEQ ID NO: 596) | |
| 597 | AGAGTCTCTCAGCTGGTACAGTTTTAGAGCTA | 3′ pegRNA |
| | GAAATAGCAAGTTAAAATAAGGCTAGTCCGT | (Pa01_2_39 nt_RTT2) |
| | TATCAACTTGAAAAAGTGGCACCGAGTCGGT | |
| | GCCCGTGACCTACATGCTCGAAGGGCGTATGC | |
| | GCCACGAAGACCAGCTGAGAGA (SEQ ID NO: | |
| | 597) | |
| 598 | AGAGTCTCTCAGCTGGTACAGTTTTAGAGCTA | 3′ pegRNA |
| | GAAATAGCAAGTTAAAATAAGGCTAGTCCGT | (Bxb1_39 nt_RTT2); contains |
| | TATCAACTTGAAAAAGTGGCACCGAGTCGGT | 3′ end modification TTTT |
| | GCCCGTGACCTACATGCTCGAAGGGCGTATGC | |
| | GCCACGAAGACCAGCTGAGAGATTTT (SEQ ID | |
| | NO: 598) | |
| 599 | AGAGTCTCTCAGCTGGTACAGTTTTAGAGCTA | 3′ pegRNA |
| | GAAATAGCAAGTTAAAATAAGGCTAGTCCGT | (Pa01_1_27 nt_RTT2) |
| | TATCAACTTGAAAAAGTGGCACCGAGTCGGT | |
| | GCCCTACATGCTCGAAGGGCGTATGCGCCACC | |
| | AGCTGAGAGA (SEQ ID NO: 599) | |
| 600 | AGAGTCTCTCAGCTGGTACAGTTTTAGAGCTA | 3′ pegRNA |
| | GAAATAGCAAGTTAAAATAAGGCTAGTCCGT | (Pa01_1_27 nt_RTT2), |
| | TATCAACTTGAAAAAGTGGCACCGAGTCGGT | contains 3′ end modification |
| | GCCCTACATGCTCGAAGGGCGTATGCGCCACC | TTTT |
| | AGCTGAGAGATTTT (SEQ ID NO: 600) | |
| 601 | AGAGTCTCTCAGCTGGTACAGTTTTAGAGCTA | 3′ pegRNA |
| | GAAATAGCAAGTTAAAATAAGGCTAGTCCGT | (Pa01_1_33 nt_RTT2) |
| | TATCAACTTGAAAAAGTGGCACCGAGTCGGT | |
| | GCCCGTGACCTACATGCTCGAAGGGCGTATGC | |
| | GCCACCAGCTGAGAGA (SEQ ID NO: 601) | |
| 602 | AGAGTCTCTCAGCTGGTACAGTTTTAGAGCTA | 3′ pegRNA |
| | GAAATAGCAAGTTAAAATAAGGCTAGTCCGT | (Pa01_1_33 nt_RTT2), |
| | TATCAACTTGAAAAAGTGGCACCGAGTCGGT | contains 3′ end modification |
| | GCCCGTGACCTACATGCTCGAAGGGCGTATGC | TTTT |
| | GCCACCAGCTGAGAGATTTT (SEQ ID NO: 602) | |
| 1127 | AGAGTCTCTCAGCTGGTACAGTTTTAGAGCTA | 3′ pegRNA(Bxb1_38 nt |
| | GAAATAGCAAGTTAAAATAAGGCTAGTCCGT | RTT1). contains 3′ end |
| | TATCAACTTGAAAAAGTGGCACCGAGTCGGT | modification TTTT |
| | GCATGATCCTGACGACGGAGACCGCCGTCGT | |
| | CGACAAGCCACCAGCTGATTTT (SEQ ID NO: | |
| | 1127) | |
| 1136 | AGAGTCTCTCAGCTGGTACAGTTTTAGAGCTA | 3′ pegRNA (Bxb1_30 nt |
| | GAAATAGCAAGTTAAAATAAGGCTAGTCCGT | RTT1), contains 3′ end |
| | TATCAACTTGAAAAAGTGGCACCGAGTCGGT | modification TTTT |
| | GCGACGACGGAGACCGCCGTCGTCGACAAGC | |
| | CACCAGCTGAGATTTT (SEQ ID NO: 1136) | |
| 1139 | AGAGTCTCTCAGCTGGTACAGTTTTAGAGCTA | 3′ pegRNA (Bxb1_29 nt |
| | GAAATAGCAAGTTAAAATAAGGCTAGTCCGT | RTT2), contains 3′ end |
| | TATCAACTTGAAAAAGTGGCACCGAGTCGGT | modification TTTT |
| | GCACGACGGCGGTCTCCGTCGTCAGGATCATA | |
| | CCAGCTGAGAGATTTT (SEQ ID NO: 1139) | |
| 1141 | AGAGTCTCTCAGCTGGTACAGTTTTAGAGCTA | 3′ pegRNA ((Bxb1_29 nt |
| | GAAATAGCAAGTTAAAATAAGGCTAGTCCGT | RTT2), contains 3′ end |
| | TATCAACTTGAAAAAGTGGCACCGAGTCGGT | modification TTTT |
| | GCACGACGGCGGTCTCCGTCGTCAGGATCATA | |
| | CCAGCTGAGATTTT (SEQ ID NO: 1141) | |

TABLE 14

| Sequence | | |
|---|---|---|
| Number | Sequence | Description |
| 230 | TGGTACACGGCAGGGTC (SEQ ID NO: 230) | 3′ PEgRNA spacer (AGG |
| | | PAM) |
| 231 | CTGGTACACGGCAGGGTC (SEQ ID NO: 231) | 3′ PEgRNA spacer (AGG |
| | | PAM) |
| 232 | GCTGGTACACGGCAGGGTC (SEQ ID NO: 232) | 3′ PEgRNA spacer (AGG |
| | | PAM) |
| 233 | AGCTGGTACACGGCAGGGTC (SEQ ID NO: 233) | 3′ PERNA spacer (AGG |
| | | PAM) |
| 234 | CAGCTGGTACACGGCAGGGTC (SEQ ID NO: | 3′ PEgRNA spacer (AGG |
| | 234) | PAM) |
| 235 | TCAGCTGGTACACGGCAGGGTC (SEQ ID NO: | 3′ PEgRNA spacer (AGG |
| | 235) | PAM) |
| 236 | CCTGC | 3′ PBS |
| 237 | CCTGCC | 3′ PBS |
| 238 | CCTGCCG | 3′ PBS |
| 239 | CCTGCCGT | 3′ PBS |
| 240 | CCTGCCGTG | 3′ PBS |
| 241 | CCTGCCGTGT (SEQ ID NO: 241) | 3′ PBS |
| 242 | CCTGCCGTGTA (SEQ ID NO: 242) | 3′ PBS |
| 243 | CCTGCCGTGTAC (SEQ ID NO: 243) | 3′ PBS |
| 244 | CCTGCCGTGTACC (SEQ ID NO: 244) | 3′ PBS |
| 245 | CCTGCCGTGTACCA (SEQ ID NO: 245) | 3′ PBS |
| 246 | CCTGCCGTGTACCAG (SEQ ID NO: 246) | 3′ PBS |
| 247 | CCTGCCGTGTACCAGC (SEQ ID NO: 247) | 3′ PBS |
| 248 | CCTGCCGTGTACCAGCT (SEQ ID NO: 248) | 3′ PBS |
| 249 | CCTGCCGTGTACCAGCTG (SEQ ID NO: 249) | 3′ PBS |
| 250 | CCTGCCGTGTACCAGCTGA (SEQ ID NO: 250) | 3′ PBS |
| 251 | AGCTGGTACACGGCAGGGTCGTTTTAGAGCTA | 3′ pegRNA |
| | GAAATAGCAAGTTAAAATAAGGCTAGTCCGT | (Bxb1_38 nt_RTT2) |
| | TATCAACTTGAAAAAGTGGCACCGAGTCGGT | |
| | GCGGCTTGTCGACGACGGCGGTCTCCGTCGTC | |
| | AGGATCATCCTGCCGT (SEQ ID NO: 251) | |
| 252 | AGCTGGTACACGGCAGGGTCGTTTTAGAGCTA | 3′ pegRNA |
| | GAAATAGCAAGTTAAAATAAGGCTAGTCCGT | (Bxb1_38 nt_RTT2) |
| | TATCAACTTGAAAAAGTGGCACCGAGTCGGT | |
| | GCGGCTTGTCGACGACGGCGGTCTCCGTCGTC | |
| | AGGATCATCCTGCCGTGT (SEQ ID NO: 252) | |
| 253 | AGCTGGTACACGGCAGGGTCGTTTTAGAGCTA | 3′ pegRNA |
| | GAAATAGCAAGTTAAAATAAGGCTAGTCCGT | (Bxb1_38 nt_RTT2); contains |
| | TATCAACTTGAAAAAGTGGCACCGAGTCGGT | 3′ end modification TTTT |
| | GCGGCTTGTCGACGACGGCGGTCTCCGTCGTC | |
| | AGGATCATCCTGCCGTTTTT (SEQ ID NO: 253) | |
| 254 | AGCTGGTACACGGCAGGGTCGTTTTAGAGCTA | 3′ pegRNA |
| | GAAATAGCAAGTTAAAATAAGGCTAGTCCGT | (Bxb1_38 nt_RTT2) |
| | TATCAACTTGAAAAAGTGGCACCGAGTCGGT | |
| | GCGGCTTGTCGACGACGGCGGTCTCCGTCGTC | |
| | AGGATCATCCTGCCGTGTAC (SEQ ID NO: 254) | |
| 255 | AGCTGGTACACGGCAGGGTCGTTTTAGAGCTA | 3′ pegRNA |
| | GAAATAGCAAGTTAAAATAAGGCTAGTCCGT | (Bxb1_38 nt_RTT2); contains |
| | TATCAACTTGAAAAAGTGGCACCGAGTCGGT | 3′ end modification TTTT |
| | GCGGCTTGTCGACGACGGCGGTCTCCGTCGTC | |
| | AGGATCATCCTGCCGTGTTTTT (SEQ ID NO: | |
| | 255) | |
| 256 | AGCTGGTACACGGCAGGGTCGTTTTAGAGCTA | 3′ pegRNA |
| | GAAATAGCAAGTTAAAATAAGGCTAGTCCGT | (Bxb1_38 nt_RTT2); contains |
| | TATCAACTTGAAAAAGTGGCACCGAGTCGGT | 3′ end modification TTTT |
| | GCGGCTTGTCGACGACGGCGGTCTCCGTCGTC | |
| | AGGATCATCCTGCCGTGTACTTTT (SEQ ID NO: | |
| | 256) | |

TABLE 15

| Sequence | | |
|---|---|---|
| Number | Sequence | Description |
| 257 | TGTGGGACAAGAGGATC (SEQ ID NO: 257) | 3′ PEgRNA spacer (AGG |
| | | PAM) |
| 258 | CTGTGGGACAAGAGGATC (SEQ ID NO: 258) | 3′ PEgRNA spacer (AGG |
| | | PAM) |
| 259 | TCTGTGGGACAAGAGGATC (SEQ ID NO: 259) | 3′ PEgRNA spacer (AGG |
| | | PAM) |
| 260 | ATCTGTGGGACAAGAGGATC (SEQ ID NO: 260) | 3′ PEgRNA spacer (AGG |
| | | PAM) |
| 261 | TATCTGTGGGACAAGAGGATC (SEQ ID NO: | 3′ PEgRNA spacer (AGG |
| | 261) | PAM) |
| 262 | ATATCTGTGGGACAAGAGGATC (SEQ ID NO: | 3′ PERNA spacer (AGG |
| | 262) | PAM) |
| 263 | CCTCT | 3′ PBS |
| 264 | CCTCTT | 3′ PBS |
| 265 | CCTCTTG | 3′ PBS |
| 266 | CCTCTTGT | 3′ PBS |
| 267 | CCTCTTGTC | 3′ PBS |
| 268 | CCTCTTGTCC (SEQ ID NO: 268) | 3′ PBS |
| 269 | CCTCTTGTCCC (SEQ ID NO: 269) | 3′ PBS |
| 270 | CCTCTTGTCCCA (SEQ ID NO: 270) | 3′ PBS |
| 271 | CCTCTTGTCCCAC (SEQ ID NO: 271) | 3′ PBS |
| 272 | CCTCTTGTCCCACA (SEQ ID NO: 272) | 3′ PBS |
| 273 | CCTCTTGTCCCACAG (SEQ ID NO: 273) | 3′ PBS |
| 274 | CCTCTTGTCCCACAGA (SEQ ID NO: 274) | 3′ PBS |
| 275 | CCTCTTGTCCCACAGAT (SEQ ID NO: 275) | 3′ PBS |
| 276 | CCTCTTGTCCCACAGATA (SEQ ID NO: 276) | 3′ PBS |
| 277 | CCTCTTGTCCCACAGATAT (SEQ ID NO: 277) | 3′ PBS |
| 278 | ATCTGTGGGACAAGAGGATCGTTTTAGAGCTA | 3′ pegRNA |
| | GAAATAGCAAGTTAAAATAAGGCTAGTCCGT | (Bxb1_38 nt_RTT2) |
| | TATCAACTTGAAAAAGTGGCACCGAGTCGGT | |
| | GCGGCTTGTCGACGACGGCGGTCTCCGTCGTC | |
| | AGGATCATCCTCTTGTC (SEQ ID NO: 278) | |
| 279 | ATCTGTGGGACAAGAGGATCGTTTTAGAGCTA | 3′ pegRNA |
| | GAAATAGCAAGTTAAAATAAGGCTAGTCCGT | (Bxb1_38 nt_RTT2) |
| | TATCAACTTGAAAAAGTGGCACCGAGTCGGT | |
| | GCGGCTTGTCGACGACGGCGGTCTCCGTCGTC | |
| | AGGATCATCCTCTTGTCCC (SEQ ID NO: 279) | |
| 280 | ATCTGTGGGACAAGAGGATCGTTTTAGAGCTA | 3′ pegRNA |
| | GAAATAGCAAGTTAAAATAAGGCTAGTCCGT | (Bxb1_38 nt_RTT2); contains |
| | TATCAACTTGAAAAAGTGGCACCGAGTCGGT | 3′ end modification TTTT |
| | GCGGCTTGTCGACGACGGCGGTCTCCGTCGTC | |
| | AGGATCATCCTCTTGTCTTTT (SEQ ID NO: 280) | |
| 281 | ATCTGTGGGACAAGAGGATCGTTTTAGAGCTA | 3′ pegRNA |
| | GAAATAGCAAGTTAAAATAAGGCTAGTCCGT | (Bxb1_38 nt_RTT2) |
| | TATCAACTTGAAAAAGTGGCACCGAGTCGGT | |
| | GCGGCTTGTCGACGACGGCGGTCTCCGTCGTC | |
| | AGGATCATCCTCTTGTCCCAC (SEQ ID NO: 281) | |
| 282 | ATCTGTGGGACAAGAGGATCGTTTTAGAGCTA | 3′ pegRNA |
| | GAAATAGCAAGTTAAAATAAGGCTAGTCCGT | (Bxb1_38 nt_RTT2); contains |
| | TATCAACTTGAAAAAGTGGCACCGAGTCGGT | 3′ end modification TTTT |
| | GCGGCTTGTCGACGACGGCGGTCTCCGTCGTC | |
| | AGGATCATCCTCTTGTCCCTTTT (SEQ ID NO: | |
| | 282) | |
| 283 | ATCTGTGGGACAAGAGGATCGTTTTAGAGCTA | 3′ pegRNA |
| | GAAATAGCAAGTTAAAATAAGGCTAGTCCGT | (Bxb1_38 nt_RTT2); contains |
| | TATCAACTTGAAAAAGTGGCACCGAGTCGGT | 3′ end modification TTTT |
| | GCGGCTTGTCGACGACGGCGGTCTCCGTCGTC | |
| | AGGATCATCCTCTTGTCCCACTTTT (SEQ ID | |
| | NO: 283) | |

TABLE 16

| Sequence | | |
|---|---|---|
| Number | Sequence | Description |
| 284 | TCAGCTGGTACACGGCA (SEQ ID NO: 284) | 3′ PEgRNA spacer (GGG |
| | | PAM) |
| 285 | CTCAGCTGGTACACGGCA (SEQ ID NO: 285) | 3′ PEgRNA spacer (GGG |
| | | PAM) |
| 286 | TCTCAGCTGGTACACGGCA (SEQ ID NO: 286) | 3′ PEgRNA spacer (GGG |
| | | PAM) |
| 287 | CTCTCAGCTGGTACACGGCA (SEQ ID NO: 287) | 3′ PEgRNA spacer (GGG |
| | | PAM) |
| 288 | TCTCTCAGCTGGTACACGGCA (SEQ ID NO: | 3′ PEgRNA spacer (GGG |
| | 288) | PAM) |
| 289 | GTCTCTCAGCTGGTACACGGCA (SEQ ID NO: | 3′ PEgRNA spacer (GGG |
| | 289) | PAM) |
| 290 | CGTGT | 3′ PBS |
| 291 | CGTGTA | 3′ PBS |
| 292 | CGTGTAC | 3′ PBS |
| 293 | CGTGTACC | 3′ PBS |
| 294 | CGTGTACCA | 3′ PBS |
| 295 | CGTGTACCAG (SEQ ID NO: 295) | 3′ PBS |
| 296 | CGTGTACCAGC (SEQ ID NO: 296) | 3′ PBS |
| 297 | CGTGTACCAGCT (SEQ ID NO: 297) | 3′ PBS |
| 298 | CGTGTACCAGCTG (SEQ ID NO: 298) | 3′ PBS |
| 299 | CGTGTACCAGCTGA (SEQ ID NO: 299) | 3′ PBS |
| 300 | CGTGTACCAGCTGAG (SEQ ID NO: 300) | 3′ PBS |
| 301 | CGTGTACCAGCTGAGA (SEQ ID NO: 301) | 3′ PBS |
| 302 | CGTGTACCAGCTGAGAG (SEQ ID NO: 302) | 3′ PBS |
| 303 | CGTGTACCAGCTGAGAGA (SEQ ID NO: 303) | 3′ PBS |
| 304 | CGTGTACCAGCTGAGAGAC (SEQ ID NO: 304) | 3′ PBS |
| 305 | CTCTCAGCTGGTACACGGCAGTTTTAGAGCTA | 3′ pegRNA |
| | GAAATAGCAAGTTAAAATAAGGCTAGTCCGT | (Bxb1_38 nt_RTT2) |
| | TATCAACTTGAAAAAGTGGCACCGAGTCGGT | |
| | GCGGCTTGTCGACGACGGCGGTCTCCGTCGTC | |
| | AGGATCATCGTGTACC (SEQ ID NO: 305) | |
| 306 | CTCTCAGCTGGTACACGGCAGTTTTAGAGCTA | 3′ pegRNA |
| | GAAATAGCAAGTTAAAATAAGGCTAGTCCGT | (Bxb1_38 nt_RTT2) |
| | TATCAACTTGAAAAAGTGGCACCGAGTCGGT | |
| | GCGGCTTGTCGACGACGGCGGTCTCCGTCGTC | |
| | AGGATCATCGTGTACCAG (SEQ ID NO: 306) | |
| 307 | CTCTCAGCTGGTACACGGCAGTTTTAGAGCTA | 3′ pegRNA |
| | GAAATAGCAAGTTAAAATAAGGCTAGTCCGT | (Bxb1_38 nt_RTT2); contains |
| | TATCAACTTGAAAAAGTGGCACCGAGTCGGT | 3′ end modification TTTT |
| | GCGGCTTGTCGACGACGGCGGTCTCCGTCGTC | |
| | AGGATCATCGTGTACCTTTT (SEQ ID NO: 307) | |
| 308 | CTCTCAGCTGGTACACGGCAGTTTTAGAGCTA | 3′ pegRNA |
| | GAAATAGCAAGTTAAAATAAGGCTAGTCCGT | (Bxb1_38 nt_RTT2) |
| | TATCAACTTGAAAAAGTGGCACCGAGTCGGT | |
| | GCGGCTTGTCGACGACGGCGGTCTCCGTCGTC | |
| | AGGATCATCGTGTACCAGCT (SEQ ID NO: 308) | |
| 309 | CTCTCAGCTGGTACACGGCAGTTTTAGAGCTA | 3′ pegRNA |
| | GAAATAGCAAGTTAAAATAAGGCTAGTCCGT | (Bxb1_38 nt_RTT2); contains |
| | TATCAACTTGAAAAAGTGGCACCGAGTCGGT | 3′ end modification TTTT |
| | GCGGCTTGTCGACGACGGCGGTCTCCGTCGTC | |
| | AGGATCATCGTGTACCAGTTTT (SEQ ID NO: | |
| | 309) | |
| 310 | CTCTCAGCTGGTACACGGCAGTTTTAGAGCTA | 3′ pegRNA |
| | GAAATAGCAAGTTAAAATAAGGCTAGTCCGT | (Bxb1_38 nt_RTT2); contains |
| | TATCAACTTGAAAAAGTGGCACCGAGTCGGT | 3′ end modification TTTT |
| | GCGGCTTGTCGACGACGGCGGTCTCCGTCGTC | |
| | AGGATCATCGTGTACCAGCTTTTT (SEQ ID NO: | |
| | 310) | |

TABLE 17

| Sequence | | |
|---|---|---|
| Number | Sequence | Description |
| 311 | GATATCTGTGGGACAAG (SEQ ID NO: 311) | 3′ PEgRNA spacer (AGG |
| | | PAM) |
| 312 | GGATATCTGTGGGACAAG (SEQ ID NO: 312) | 3′ PEgRNA spacer (AGG |
| | | PAM) |
| 313 | TGGATATCTGTGGGACAAG (SEQ ID NO: 313) | 3′ PEgRNA spacer (AGG |
| | | PAM) |
| 314 | CTGGATATCTGTGGGACAAG (SEQ ID NO: 314) | 3′ PEgRNA spacer (AGG |
| | | PAM) |
| 315 | TCTGGATATCTGTGGGACAAG (SEQ ID NO: | 3′ PERNA spacer (AGG |
| | 315) | PAM) |
| 316 | TTCTGGATATCTGTGGGACAAG (SEQ ID NO: | 3′ PEgRNA spacer (AGG |
| | 316) | PAM) |
| 317 | GTCCC | 3′ PBS |
| 318 | GTCCCA | 3′ PBS |
| 319 | GTCCCAC | 3′ PBS |
| 320 | GTCCCACA | 3′ PBS |
| 321 | GTCCCACAG | 3′ PBS |
| 322 | GTCCCACAGA (SEQ ID NO: 322) | 3′ PBS |
| 323 | GTCCCACAGAT (SEQ ID NO: 323) | 3′ PBS |
| 324 | GTCCCACAGATA (SEQ ID NO: 324) | 3′ PBS |
| 325 | GTCCCACAGATAT (SEQ ID NO: 325) | 3′ PBS |
| 326 | GTCCCACAGATATC (SEQ ID NO: 326) | 3′ PBS |
| 327 | GTCCCACAGATATCC (SEQ ID NO: 327) | 3′ PBS |
| 328 | GTCCCACAGATATCCA (SEQ ID NO: 328) | 3′ PBS |
| 329 | GTCCCACAGATATCCAG (SEQ ID NO: 329) | 3′ PBS |
| 330 | GTCCCACAGATATCCAGA (SEQ ID NO: 330) | 3′ PBS |
| 331 | GTCCCACAGATATCCAGAA (SEQ ID NO: 331) | 3′ PBS |
| 332 | CTGGATATCTGTGGGACAAGGTTTTAGAGCTA | 3′ pegRNA |
| | GAAATAGCAAGTTAAAATAAGGCTAGTCCGT | (Bxb1_38 nt_RTT2) |
| | TATCAACTTGAAAAAGTGGCACCGAGTCGGT | |
| | GCGGCTTGTCGACGACGGCGGTCTCCGTCGTC | |
| | AGGATCATGTCCCACAG (SEQ ID NO: 332) | |
| 333 | CTGGATATCTGTGGGACAAGGTTTTAGAGCTA | 3′ pegRNA |
| | GAAATAGCAAGTTAAAATAAGGCTAGTCCGT | (Bxb1_38 nt_RTT2) |
| | TATCAACTTGAAAAAGTGGCACCGAGTCGGT | |
| | GCGGCTTGTCGACGACGGCGGTCTCCGTCGTC | |
| | AGGATCATGTCCCACAGAT (SEQ ID NO: 333) | |
| 334 | CTGGATATCTGTGGGACAAGGTTTTAGAGCTA | 3′ pegRNA |
| | GAAATAGCAAGTTAAAATAAGGCTAGTCCGT | (Bxb1_38 nt_RTT2); contains |
| | TATCAACTTGAAAAAGTGGCACCGAGTCGGT | 3′ end modification TTTT |
| | GCGGCTTGTCGACGACGGCGGTCTCCGTCGTC | |
| | AGGATCATGTCCCACAGTTTT (SEQ ID NO: | |
| | 334) | |
| 335 | CTGGATATCTGTGGGACAAGGTTTTAGAGCTA | 3′ pegRNA |
| | GAAATAGCAAGTTAAAATAAGGCTAGTCCGT | (Bxb1_38 nt_RTT2) |
| | TATCAACTTGAAAAAGTGGCACCGAGTCGGT | |
| | GCGGCTTGTCGACGACGGCGGTCTCCGTCGTC | |
| | AGGATCATGTCCCACAGATAT (SEQ ID NO: | |
| | 335) | |
| 336 | CTGGATATCTGTGGGACAAGGTTTTAGAGCTA | 3′ pegRNA |
| | GAAATAGCAAGTTAAAATAAGGCTAGTCCGT | (Bxb1_38 nt_RTT2); contains |
| | TATCAACTTGAAAAAGTGGCACCGAGTCGGT | 3′ end modification TTTT |
| | GCGGCTTGTCGACGACGGCGGTCTCCGTCGTC | |
| | AGGATCATGTCCCACAGATTTTT (SEQ ID NO: | |
| | 336) | |
| 337 | CTGGATATCTGTGGGACAAGGTTTTAGAGCTA | 3′ pegRNA |
| | GAAATAGCAAGTTAAAATAAGGCTAGTCCGT | (Bxb1_38 nt_RTT2); contains |
| | TATCAACTTGAAAAAGTGGCACCGAGTCGGT | 3′ end modification TTTT |
| | GCGGCTTGTCGACGACGGCGGTCTCCGTCGTC | |
| | AGGATCATGTCCCACAGATATTTTT (SEQ ID | |
| | NO: 337) | |

TABLE 18

| Sequence | | |
|---|---|---|
| Number | Sequence | Description |
| 338 | AATCAAAATCGGTGAAT (SEQ ID NO: 338) | 3′ PEgRNA spacer (AGG |
| | | PAM) |
| 339 | GAATCAAAATCGGTGAAT (SEQ ID NO: 339) | 3′ PEgRNA spacer (AGG |
| | | PAM) |
| 340 | AGAATCAAAATCGGTGAAT (SEQ ID NO: 340) | 3′ PEgRNA spacer (AGG |
| | | PAM) |
| 341 | GAGAATCAAAATCGGTGAAT (SEQ ID NO: 341) | 3′ PEgRNA spacer (AGG |
| | | PAM) |
| 342 | TGAGAATCAAAATCGGTGAAT (SEQ ID NO: | 3′ PEgRNA spacer (AGG |
| | 342) | PAM) |
| 343 | TTGAGAATCAAAATCGGTGAAT (SEQ ID NO: | 3′ PEgRNA spacer (AGG |
| | 343) | PAM) |
| 344 | CACCG | 3′ PBS |
| 345 | CACCGA | 3′ PBS |
| 346 | CACCGAT | 3′ PBS |
| 347 | CACCGATT | 3′ PBS |
| 348 | CACCGATTT | 3′ PBS |
| 349 | CACCGATTTT (SEQ ID NO: 349) | 3′ PBS |
| 350 | CACCGATTTTG (SEQ ID NO: 350) | 3′ PBS |
| 351 | CACCGATTTTGA (SEQ ID NO: 351) | 3′ PBS |
| 352 | CACCGATTTTGAT (SEQ ID NO: 352) | 3′ PBS |
| 353 | CACCGATTTTGATT (SEQ ID NO: 353) | 3′ PBS |
| 354 | CACCGATTTTGATTC (SEQ ID NO: 354) | 3′ PBS |
| 355 | CACCGATTTTGATTCT (SEQ ID NO: 355) | 3′ PBS |
| 356 | CACCGATTTTGATTCTC (SEQ ID NO: 356) | 3′ PBS |
| 357 | CACCGATTTTGATTCTCA (SEQ ID NO: 357) | 3′ PBS |
| 358 | CACCGATTTTGATTCTCAA (SEQ ID NO: 358) | 3′ PBS |
| 359 | GAGAATCAAAATCGGTGAATGTTTTAGAGCT | 3′ pegRNA |
| | AGAAATAGCAAGTTAAAATAAGGCTAGTCCG | (Bxb1_38 nt_RTT2) |
| | TTATCAACTTGAAAAAGTGGCACCGAGTCGGT | |
| | GCGGCTTGTCGACGACGGCGGTCTCCGTCGTC | |
| | AGGATCATCACCGATTTTG (SEQ ID NO: 359) | |
| 360 | GAGAATCAAAATCGGTGAATGTTTTAGAGCT | 3′ pegRNA |
| | AGAAATAGCAAGTTAAAATAAGGCTAGTCCG | (Bxb1_38 nt_RTT2) |
| | TTATCAACTTGAAAAAGTGGCACCGAGTCGGT | |
| | GCGGCTTGTCGACGACGGCGGTCTCCGTCGTC | |
| | AGGATCATCACCGATTTTGATT (SEQ ID NO: | |
| | 360) | |
| 361 | GAGAATCAAAATCGGTGAATGTTTTAGAGCT | 3′ pegRNA |
| | AGAAATAGCAAGTTAAAATAAGGCTAGTCCG | (Bxb1_38 nt_RTT2); contains |
| | TTATCAACTTGAAAAAGTGGCACCGAGTCGGT | 3′ end modification TTTT |
| | GCGGCTTGTCGACGACGGCGGTCTCCGTCGTC | |
| | AGGATCATCACCGATTTTGTTTT (SEQ ID NO: | |
| | 361) | |
| 362 | GAGAATCAAAATCGGTGAATGTTTTAGAGCT | 3′ pegRNA |
| | AGAAATAGCAAGTTAAAATAAGGCTAGTCCG | (Bxb1_38 nt_RTT2) |
| | TTATCAACTTGAAAAAGTGGCACCGAGTCGGT | |
| | GCGGCTTGTCGACGACGGCGGTCTCCGTCGTC | |
| | AGGATCATCACCGATTTTGATTCT (SEQ ID NO: | |
| | 362) | |
| 363 | GAGAATCAAAATCGGTGAATGTTTTAGAGCT | 3′ pegRNA |
| | AGAAATAGCAAGTTAAAATAAGGCTAGTCCG | (Bxb1_38 nt_RTT2); contains |
| | TTATCAACTTGAAAAAGTGGCACCGAGTCGGT | 3′ end modification TTTT |
| | GCGGCTTGTCGACGACGGCGGTCTCCGTCGTC | |
| | AGGATCATCACCGATTTTGATTTTTT (SEQ ID | |
| | NO: 363) | |
| 364 | GAGAATCAAAATCGGTGAATGTTTTAGAGCT | 3′ pegRNA |
| | AGAAATAGCAAGTTAAAATAAGGCTAGTCCG | (Bxb1_38 nt_RTT2); contains |
| | TTATCAACTTGAAAAAGTGGCACCGAGTCGGT | 3′ end modification TTTT |
| | GCGGCTTGTCGACGACGGCGGTCTCCGTCGTC | |
| | AGGATCATCACCGATTTTGATTCTTTTT (SEQ | |
| | ID NO: 364) | |

TABLE 19

| Sequence | | |
|---|---|---|
| Number | Sequence | Description |
| 365 | GGTACACGGCAGGGTCA (SEQ ID NO: 365) | 3′ PEgRNA spacer (GGG |
| | | PAM) |
| 366 | TGGTACACGGCAGGGTCA (SEQ ID NO: | 3′ PEgRNA spacer (GGG |
| | 366) | PAM) |
| 367 | CTGGTACACGGCAGGGTCA (SEQ ID NO: | 3′ PEgRNA spacer (GGG |
| | 367) | PAM) |
| 368 | GCTGGTACACGGCAGGGTCA (SEQ ID NO: | 3′ PEgRNA spacer (GGG |
| | 368) | PAM) |
| 369 | AGCTGGTACACGGCAGGGTCA (SEQ ID | 3′ PEgRNA spacer (GGG |
| | NO: 369) | PAM) |
| 370 | CAGCTGGTACACGGCAGGGTCA (SEQ ID | 3′ PEgRNA spacer (GGG |
| | NO: 370) | PAM) |
| 371 | CCCTG | 3′ PBS |
| 372 | CCCTGC | 3′ PBS |
| 373 | CCCTGCC | 3′ PBS |
| 374 | CCCTGCCG | 3′ PBS |
| 375 | CCCTGCCGT | 3′ PBS |
| 376 | CCCTGCCGTG (SEQ ID NO: 376) | 3′ PBS |
| 377 | CCCTGCCGTGT (SEQ ID NO: 377) | 3′ PBS |
| 378 | CCCTGCCGTGTA (SEQ ID NO: 378) | 3′ PBS |
| 379 | CCCTGCCGTGTAC (SEQ ID NO: 379) | 3′ PBS |
| 380 | CCCTGCCGTGTACC (SEQ ID NO: 380) | 3′ PBS |
| 381 | CCCTGCCGTGTACCA (SEQ ID NO: 381) | 3′ PBS |
| 382 | CCCTGCCGTGTACCAG (SEQ ID NO: 382) | 3′ PBS |
| 383 | CCCTGCCGTGTACCAGC (SEQ ID NO: 383) | 3′ PBS |
| 384 | CCCTGCCGTGTACCAGCT (SEQ ID NO: 384) | 3′ PBS |
| 385 | CCCTGCCGTGTACCAGCTG (SEQ ID NO: | 3′ PBS |
| | 385) | |
| 386 | GCTGGTACACGGCAGGGTCAGTTTTAGAG | 3′ pegRNA |
| | CTAGAAATAGCAAGTTAAAATAAGGCTA | (Bxb1_29 nt_RTT2) |
| | GTCCGTTATCAACTTGAAAAAGTGGCACC | |
| | GAGTCGGTGCACGACGGCGGTCTCCGTCG | |
| | TCAGGATCATCCCTGC (SEQ ID NO: 386) | |
| 387 | GCTGGTACACGGCAGGGTCAGTTTTAGAG | 3′ pegRNA |
| | CTAGAAATAGCAAGTTAAAATAAGGCTA | (Bxb1_30 nt_RTT2) |
| | GTCCGTTATCAACTTGAAAAAGTGGCACC | |
| | GAGTCGGTGCGACGACGGCGGTCTCCGTC | |
| | GTCAGGATCATCCCTGCC (SEQ ID NO: 387) | |
| 388 | GCTGGTACACGGCAGGGTCAGTTTTAGAG | 3′ pegRNA |
| | CTAGAAATAGCAAGTTAAAATAAGGCTA | (Bxb1_29 nt_RTT2) |
| | GTCCGTTATCAACTTGAAAAAGTGGCACC | |
| | GAGTCGGTGCACGACGGCGGTCTCCGTCG | |
| | TCAGGATCATCCCTGCCGTG (SEQ ID NO: | |
| | 388) | |
| 389 | GCTGGTACACGGCAGGGTCAGTTTTAGAG | 3′ pegRNA |
| | CTAGAAATAGCAAGTTAAAATAAGGCTA | (Bxb1_30 nt_RTT2) |
| | GTCCGTTATCAACTTGAAAAAGTGGCACC | |
| | GAGTCGGTGCGACGACGGCGGTCTCCGTC | |
| | GTCAGGATCATCCCTGCCGT (SEQ ID NO: | |
| | 389) | |
| 390 | GCTGGTACACGGCAGGGTCAGTTTTAGAG | 3′ pegRNA |
| | CTAGAAATAGCAAGTTAAAATAAGGCTA | (Bxb1_30 nt_RTT2); |
| | GTCCGTTATCAACTTGAAAAAGTGGCACC | contains 3′ end |
| | GAGTCGGTGCGACGACGGCGGTCTCCGTC | modification TTTT |
| | GTCAGGATCATCCCTGCCTTTT (SEQ ID | |
| | NO: 390) | |
| 391 | GCTGGTACACGGCAGGGTCAGTTTTAGAG | 3′ pegRNA |
| | CTAGAAATAGCAAGTTAAAATAAGGCTA | (Bxb1_30 nt_RTT2) |
| | GTCCGTTATCAACTTGAAAAAGTGGCACC | |
| | GAGTCGGTGCGACGACGGCGGTCTCCGTC | |
| | GTCAGGATCATCCCTGCCGTGT (SEQ ID | |
| | NO: 391) | |
| 392 | GCTGGTACACGGCAGGGTCAGTTTTAGAG | 3′ pegRNA |
| | CTAGAAATAGCAAGTTAAAATAAGGCTA | (Bxb1_30 nt_RTT2); |
| | GTCCGTTATCAACTTGAAAAAGTGGCACC | contains 3′ end |
| | GAGTCGGTGCGACGACGGCGGTCTCCGTC | modification TTTT |
| | GTCAGGATCATCCCTGCCGTTTTT (SEQ ID | |
| | NO: 392) | |
| 393 | GCTGGTACACGGCAGGGTCAGTTTTAGAG | 3′ pegRNA |
| | CTAGAAATAGCAAGTTAAAATAAGGCTA | (Bxb1_30 nt_RTT2) |
| | GTCCGTTATCAACTTGAAAAAGTGGCACC | |
| | GAGTCGGTGCGACGACGGCGGTCTCCGTC | |
| | GTCAGGATCATCCCTGCCGTGTAC (SEQ ID | |
| | NO: 393) | |
| 394 | GCTGGTACACGGCAGGGTCAGTTTTAGAG | 3′ pegRNA |
| | CTAGAAATAGCAAGTTAAAATAAGGCTA | (Bxb1_38 nt_RTT2) |
| | GTCCGTTATCAACTTGAAAAAGTGGCACC | |
| | GAGTCGGTGCGGCTTGTCGACGACGGCGG | |
| | TCTCCGTCGTCAGGATCATCCCTGC (SEQ | |
| | ID NO: 394) | |
| 395 | GCTGGTACACGGCAGGGTCAGTTTTAGAG | 3′ pegRNA |
| | CTAGAAATAGCAAGTTAAAATAAGGCTA | (Bxb1_30 nt_RTT2); |
| | GTCCGTTATCAACTTGAAAAAGTGGCACC | contains 3′ end |
| | GAGTCGGTGCGACGACGGCGGTCTCCGTC | modification TTTT |
| | GTCAGGATCATCCCTGCCGTGTTTTT (SEQ | |
| | ID NO: 395) | |
| 396 | GCTGGTACACGGCAGGGTCAGTTTTAGAG | 3′ pegRNA |
| | CTAGAAATAGCAAGTTAAAATAAGGCTA | (Bxb1_38 nt_RTT2) |
| | GTCCGTTATCAACTTGAAAAAGTGGCACC | |
| | GAGTCGGTGCGGCTTGTCGACGACGGCGG | |
| | TCTCCGTCGTCAGGATCATCCCTGCC (SEQ | |
| | ID NO: 396) | |
| 397 | GCTGGTACACGGCAGGGTCAGTTTTAGAG | 3′ pegRNA |
| | CTAGAAATAGCAAGTTAAAATAAGGCTA | (Bxb1_38 nt_RTT2) |
| | GTCCGTTATCAACTTGAAAAAGTGGCACC | |
| | GAGTCGGTGCGGCTTGTCGACGACGGCGG | |
| | TCTCCGTCGTCAGGATCATCCCTGCCG | |
| | (SEQ ID NO: 397) | |
| 398 | GCTGGTACACGGCAGGGTCAGTTTTAGAG | 3′ pegRNA |
| | CTAGAAATAGCAAGTTAAAATAAGGCTA | (Bxb1_30 nt_RTT2); |
| | GTCCGTTATCAACTTGAAAAAGTGGCACC | contains 3′ end |
| | GAGTCGGTGCGACGACGGCGGTCTCCGTC | modification TTTT |
| | GTCAGGATCATCCCTGCCGTGTACTTTT | |
| | (SEQ ID NO: 398) | |
| 399 | GCTGGTACACGGCAGGGTCAGTTTTAGAG | 3′ pegRNA |
| | CTAGAAATAGCAAGTTAAAATAAGGCTA | (Bxb1_38 nt_RTT2) |
| | GTCCGTTATCAACTTGAAAAAGTGGCACC | |
| | GAGTCGGTGCGGCTTGTCGACGACGGCGG | |
| | TCTCCGTCGTCAGGATCATCCCTGCCGT | |
| | (SEQ ID NO: 399) | |
| 400 | GCTGGTACACGGCAGGGTCAGTTTTAGAG | 3′ pegRNA |
| | CTAGAAATAGCAAGTTAAAATAAGGCTA | (Bxb1_38 nt_RTT2) |
| | GTCCGTTATCAACTTGAAAAAGTGGCACC | |
| | GAGTCGGTGCGGCTTGTCGACGACGGCGG | |
| | TCTCCGTCGTCAGGATCATCCCTGCCGTG | |
| | (SEQ ID NO: 400) | |
| 401 | GCTGGTACACGGCAGGGTCAGTTTTAGAG | 3′ pegRNA |
| | CTAGAAATAGCAAGTTAAAATAAGGCTA | (Bxb1_38 nt_RTT2); |
| | GTCCGTTATCAACTTGAAAAAGTGGCACC | contains 3′ end |
| | GAGTCGGTGCGGCTTGTCGACGACGGCGG | modification TTTT |
| | TCTCCGTCGTCAGGATCATCCCTGCCTTTT | |
| | (SEQ ID NO: 401) | |
| 402 | GCTGGTACACGGCAGGGTCAGTTTTAGAG | 3′ pegRNA |
| | CTAGAAATAGCAAGTTAAAATAAGGCTA | (Bxb1_38 nt_RTT2) |
| | GTCCGTTATCAACTTGAAAAAGTGGCACC | |
| | GAGTCGGTGCGGCTTGTCGACGACGGCGG | |
| | TCTCCGTCGTCAGGATCATCCCTGCCGTGT | |
| | (SEQ ID NO: 402) | |
| 403 | GCTGGTACACGGCAGGGTCAGTTTTAGAG | 3′ pegRNA |
| | CTAGAAATAGCAAGTTAAAATAAGGCTA | (Bxb1_38 nt_RTT2); |
| | GTCCGTTATCAACTTGAAAAAGTGGCACC | contains 3′ end |
| | GAGTCGGTGCGGCTTGTCGACGACGGCGG | modification TTTT |
| | TCTCCGTCGTCAGGATCATCCCTGCCGTTT | |
| | T (SEQ ID NO: 403) | |
| 404 | GCTGGTACACGGCAGGGTCAGTTTTAGAG | 3′ pegRNA |
| | CTAGAAATAGCAAGTTAAAATAAGGCTA | (Bxb1_38 nt_RTT2) |
| | GTCCGTTATCAACTTGAAAAAGTGGCACC | |
| | GAGTCGGTGCGGCTTGTCGACGACGGCGG | |
| | TCTCCGTCGTCAGGATCATCCCTGCCGTGT | |
| | A (SEQ ID NO: 404) | |
| 405 | GCTGGTACACGGCAGGGTCAGTTTTAGAG | 3′ pegRNA |
| | CTAGAAATAGCAAGTTAAAATAAGGCTA | (Bxb1_38 nt_RTT2); |
| | GTCCGTTATCAACTTGAAAAAGTGGCACC | contains 3′ end |
| | GAGTCGGTGCGGCTTGTCGACGACGGCGG | modification TTTT |
| | TCTCCGTCGTCAGGATCATCCCTGCCGTTT | |
| | TT (SEQ ID NO: 405) | |
| 406 | GCTGGTACACGGCAGGGTCAGTTTTAGAG | 3′ pegRNA |
| | CTAGAAATAGCAAGTTAAAATAAGGCTA | (Bxb1_38 nt_RTT2) |
| | GTCCGTTATCAACTTGAAAAAGTGGCACC | |
| | GAGTCGGTGCGGCTTGTCGACGACGGCGG | |
| | TCTCCGTCGTCAGGATCATCCCTGCCGTGT | |
| | AC (SEQ ID NO: 406) | |
| 407 | GCTGGTACACGGCAGGGTCAGTTTTAGAG | 3′ pegRNA |
| | CTAGAAATAGCAAGTTAAAATAAGGCTA | (Bxb1_38 nt_RTT2); |
| | GTCCGTTATCAACTTGAAAAAGTGGCACC | contains 3′ end |
| | GAGTCGGTGCGGCTTGTCGACGACGGCGG | modification TTTT |
| | TCTCCGTCGTCAGGATCATCCCTGCCGTGT | |
| | TTT (SEQ ID NO: 407) | |
| 408 | GCTGGTACACGGCAGGGTCAGTTTTAGAG | 3′ pegRNA |
| | CTAGAAATAGCAAGTTAAAATAAGGCTA | (Bxb1_38 nt_RTT2); |
| | GTCCGTTATCAACTTGAAAAAGTGGCACC | contains 3′ end |
| | GAGTCGGTGCGGCTTGTCGACGACGGCGG | modification TTTT |
| | TCTCCGTCGTCAGGATCATCCCTGCCGTGT | |
| | TTTT (SEQ ID NO: 408) | |
| 409 | GCTGGTACACGGCAGGGTCAGTTTTAGAG | 3′ pegRNA |
| | CTAGAAATAGCAAGTTAAAATAAGGCTA | (Bxb1_38 nt_RTT2); |
| | GTCCGTTATCAACTTGAAAAAGTGGCACC | contains 3′ end |
| | GAGTCGGTGCGGCTTGTCGACGACGGCGG | modification TTTT |
| | TCTCCGTCGTCAGGATCATCCCTGCCGTGT | |
| | ATTTT (SEQ ID NO: 409) | |
| 410 | GCTGGTACACGGCAGGGTCAGTTTTAGAG | 3′ pegRNA |
| | CTAGAAATAGCAAGTTAAAATAAGGCTA | (Bxb1_38 nt_RTT2); |
| | GTCCGTTATCAACTTGAAAAAGTGGCACC | contains 3′ end |
| | GAGTCGGTGCGGCTTGTCGACGACGGCGG | modification TTTT |
| | TCTCCGTCGTCAGGATCATCCCTGCCGTGT | |
| | ACTTTT (SEQ ID NO: 410) | |
| 1128 | GCTGGTACACGGCAGGGTCAGTTTTAGAG | 3′ pegRNA (Bxb1 |
| | CTAGAAATAGCAAGTTAAAATAAGGCTA | 38 nt_RTT1); contains 3′ |
| | GTCCGTTATCAACTTGAAAAAGTGGCACC | end modification TTTT |
| | GAGTCGGTGCATGATCCTGACGACGGAGA | |
| | CCGCCGTCGTCGACAAGCCCCCTGCCTTT | |
| | T (SEQ ID NO: 1128) | |
| 1137 | GCTGGTACACGGCAGGGTCAGTTTTAGAG | 3′ pegRNA (Bxb1 |
| | CTAGAAATAGCAAGTTAAAATAAGGCTA | 30 nt_RTT1); contains 3′ |
| | GTCCGTTATCAACTTGAAAAAGTGGCACC | end modification TTTT |
| | GAGTCGGTGCGACGACGGAGACCGCCGT | |
| | CGTCGACAAGCCCCCTGCCTTTT (SEQ ID | |
| | NO: 1137) | |
| 1138 | GCTGGTACACGGCAGGGTCAGTTTTAGAG | 3′ pegRNA (Bxb1 |
| | CTAGAAATAGCAAGTTAAAATAAGGCTA | 30 nt_RTT1); contains 3′ |
| | GTCCGTTATCAACTTGAAAAAGTGGCACC | end modification TTTT |
| | GAGTCGGTGCGACGACGGAGACCGCCGT | |
| | CGTCGACAAGCCCCCTGCCGTTTTT (SEQ | |
| | ID NO: 1138) | |
| 1140 | GCTGGTACACGGCAGGGTCAGTTTTAGAG | 3′ pegRNA (Bxb1 |
| | CTAGAAATAGCAAGTTAAAATAAGGCTA | 29 nt_RTT2); contains 3′ |
| | GTCCGTTATCAACTTGAAAAAGTGGCACC | end modification TTTT |
| | GAGTCGGTGCACGACGGCGGTCTCCGTCG | |
| | TCAGGATCATCCCTGCCGTTTT (SEQ ID | |
| | NO: 1140) | |
| 1251 | GCTGGTACACGGCAGGGTCAGTTTTAGAG | 3′ pegRNA (Bxb1 |
| | CTAGAAATAGCAAGTTAAAATAAGGCTA | 29 nt_RTT2) |
| | GTCCGTTATCAACTTGAAAAAGTGGCACC | |
| | GAGTCGGTGCACGACGGCGGTCTCCGTCG | |
| | TCAGGATCATCCCTGCCG (SEQ ID NO: | |
| | 1251) | |

TABLE 20

| Sequence | | |
|---|---|---|
| Number | Sequence | Description |
| 411 | ACAAGAGGATCAGGGTT (SEQ ID NO: 411) | 3′ PERNA spacer (AGG |
| | | PAM) |
| 412 | GACAAGAGGATCAGGGTT (SEQ ID NO: 412) | 3′ PEgRNA spacer (AGG |
| | | PAM) |
| 413 | GGACAAGAGGATCAGGGTT (SEQ ID NO: 413) | 3′ PEgRNA spacer (AGG |
| | | PAM) |
| 414 | GGGACAAGAGGATCAGGGTT (SEQ ID NO: 414) | 3′ PEgRNA spacer (AGG |
| | | PAM) |
| 415 | TGGGACAAGAGGATCAGGGTT (SEQ ID NO: | 3′ PEgRNA spacer (AGG |
| | 415) | PAM) |
| 416 | GTGGGACAAGAGGATCAGGGTT (SEQ ID NO: | 3′ PERNA spacer (AGG |
| | 416) | PAM) |
| 417 | CCTGA | 3′ PBS |
| 418 | CCTGAT | 3′ PBS |
| 419 | CCTGATC | 3′ PBS |
| 420 | CCTGATCC | 3′ PBS |
| 421 | CCTGATCCT | 3′ PBS |
| 422 | CCTGATCCTC (SEQ ID NO: 422) | 3′ PBS |
| 423 | CCTGATCCTCT (SEQ ID NO: 423) | 3′ PBS |
| 424 | CCTGATCCTCTT (SEQ ID NO: 424) | 3′ PBS |
| 425 | CCTGATCCTCTTG (SEQ ID NO: 425) | 3′ PBS |
| 426 | CCTGATCCTCTTGT (SEQ ID NO: 426) | 3′ PBS |
| 427 | CCTGATCCTCTTGTC (SEQ ID NO: 427) | 3′ PBS |
| 428 | CCTGATCCTCTTGTCC (SEQ ID NO: 428) | 3′ PBS |
| 429 | CCTGATCCTCTTGTCCC (SEQ ID NO: 429) | 3′ PBS |
| 430 | CCTGATCCTCTTGTCCCA (SEQ ID NO: 430) | 3′ PBS |
| 431 | CCTGATCCTCTTGTCCCAC (SEQ ID NO: 431) | 3′ PBS |
| 432 | GGGACAAGAGGATCAGGGTTGTTTTAGAGCT | 3′ pegRNA |
| | AGAAATAGCAAGTTAAAATAAGGCTAGTCCG | (Bxb1_38 nt_RTT2) |
| | TTATCAACTTGAAAAAGTGGCACCGAGTCGGT | |
| | GCGGCTTGTCGACGACGGCGGTCTCCGTCGTC | |
| | AGGATCATCCTGATCCT (SEQ ID NO: 432) | |
| 433 | GGGACAAGAGGATCAGGGTTGTTTTAGAGCT | 3′ pegRNA |
| | AGAAATAGCAAGTTAAAATAAGGCTAGTCCG | (Bxb1_38nt_RTT2) |
| | TTATCAACTTGAAAAAGTGGCACCGAGTCGGT | |
| | GCGGCTTGTCGACGACGGCGGTCTCCGTCGTC | |
| | AGGATCATCCTGATCCTCTT (SEQ ID NO: 433) | |
| 434 | GGGACAAGAGGATCAGGGTTGTTTTAGAGCT | 3′ pegRNA |
| | AGAAATAGCAAGTTAAAATAAGGCTAGTCCG | (Bxb1_38 nt_RTT2); contains |
| | TTATCAACTTGAAAAAGTGGCACCGAGTCGGT | 3′ end modification TTTT |
| | GCGGCTTGTCGACGACGGCGGTCTCCGTCGTC | |
| | AGGATCATCCTGATCCTTTTT (SEQ ID NO: 434) | |
| 435 | GGGACAAGAGGATCAGGGTTGTTTTAGAGCT | 3′ pegRNA |
| | AGAAATAGCAAGTTAAAATAAGGCTAGTCCG | (Bxb1_38 nt_RTT2) |
| | TTATCAACTTGAAAAAGTGGCACCGAGTCGGT | |
| | GCGGCTTGTCGACGACGGCGGTCTCCGTCGTC | |
| | AGGATCATCCTGATCCTCTTGT (SEQ ID NO: | |
| | 435) | |
| 436 | GGGACAAGAGGATCAGGGTTGTTTTAGAGCT | 3′ pegRNA |
| | AGAAATAGCAAGTTAAAATAAGGCTAGTCCG | (Bxb1_38 nt_RTT2); contains |
| | TTATCAACTTGAAAAAGTGGCACCGAGTCGGT | 3′ end modification TTTT |
| | GCGGCTTGTCGACGACGGCGGTCTCCGTCGTC | |
| | AGGATCATCCTGATCCTCTTTTTT (SEQ ID NO: | |
| | 436) | |
| 437 | GGGACAAGAGGATCAGGGTTGTTTTAGAGCT | 3′ pegRNA |
| | AGAAATAGCAAGTTAAAATAAGGCTAGTCCG | (Bxb1_38 nt_RTT2); contains |
| | TTATCAACTTGAAAAAGTGGCACCGAGTCGGT | 3′ end modification TTTT |
| | GCGGCTTGTCGACGACGGCGGTCTCCGTCGTC | |
| | AGGATCATCCTGATCCTCTTGTTTTT (SEQ ID | |
| | NO: 437) | |

TABLE 21

| Sequence | | |
|---|---|---|
| Number | Sequence | Description |
| 438 | AGGGTTCTGGATATCTG (SEQ ID NO: 438) | 3′ PEgRNA spacer (TGG |
| | | PAM) |
| 439 | CAGGGTTCTGGATATCTG (SEQ ID NO: 439) | 3′ PEgRNA spacer (TGG |
| | | PAM) |
| 440 | TCAGGGTTCTGGATATCTG (SEQ ID NO: 440) | 3′ PEgRNA spacer (TGG |
| | | PAM) |
| 441 | GTCAGGGTTCTGGATATCTG (SEQ ID NO: 441) | 3′ PEgRNA spacer (TGG |
| | | PAM) |
| 442 | GGTCAGGGTTCTGGATATCTG (SEQ ID NO: | 3′ PEgRNA spacer (TGG |
| | 442) | PAM) |
| 443 | GGGTCAGGGTTCTGGATATCTG (SEQ ID NO: | 3′ PERNA spacer (TGG |
| | 443) | PAM) |
| 444 | ATATC | 3′ PBS |
| 445 | ATATCC | 3′ PBS |
| 446 | ATATCCA | 3′ PBS |
| 447 | ATATCCAG | 3′ PBS |
| 448 | ATATCCAGA | 3′ PBS |
| 449 | ATATCCAGAA (SEQ ID NO: 449) | 3′ PBS |
| 450 | ATATCCAGAAC (SEQ ID NO: 450) | 3′ PBS |
| 451 | ATATCCAGAACC (SEQ ID NO: 451) | 3′ PBS |
| 452 | ATATCCAGAACCC (SEQ ID NO: 452) | 3′ PBS |
| 453 | ATATCCAGAACCCT (SEQ ID NO: 453) | 3′ PBS |
| 454 | ATATCCAGAACCCTG (SEQ ID NO: 454) | 3′ PBS |
| 455 | ATATCCAGAACCCTGA (SEQ ID NO: 455) | 3′ PBS |
| 456 | ATATCCAGAACCCTGAC (SEQ ID NO: 456) | 3′ PBS |
| 457 | ATATCCAGAACCCTGACC (SEQ ID NO: 457) | 3′ PBS |
| 458 | ATATCCAGAACCCTGACCC (SEQ ID NO: 458) | 3′ PBS |
| 459 | GTCAGGGTTCTGGATATCTGGTTTTAGAGCTA | 3′ pegRNA |
| | GAAATAGCAAGTTAAAATAAGGCTAGTCCGT | (Bxb1_38 nt_RTT2) |
| | TATCAACTTGAAAAAGTGGCACCGAGTCGGT | |
| | GCGGCTTGTCGACGACGGCGGTCTCCGTCGTC | |
| | AGGATCATATATCCAGAA (SEQ ID NO: 459) | |
| 460 | GTCAGGGTTCTGGATATCTGGTTTTAGAGCTA | 3′ pegRNA |
| | GAAATAGCAAGTTAAAATAAGGCTAGTCCGT | (Bxb1_38 nt_RTT2) |
| | TATCAACTTGAAAAAGTGGCACCGAGTCGGT | |
| | GCGGCTTGTCGACGACGGCGGTCTCCGTCGTC | |
| | AGGATCATATATCCAGAACC (SEQ ID NO: 460) | |
| 461 | GTCAGGGTTCTGGATATCTGGTTTTAGAGCTA | 3′ pegRNA |
| | GAAATAGCAAGTTAAAATAAGGCTAGTCCGT | (Bxb1_38 nt_RTT2); contains |
| | TATCAACTTGAAAAAGTGGCACCGAGTCGGT | 3′ end modification TTTT |
| | GCGGCTTGTCGACGACGGCGGTCTCCGTCGTC | |
| | AGGATCATATATCCAGAATTTT (SEQ ID NO: | |
| | 461) | |
| 462 | GTCAGGGTTCTGGATATCTGGTTTTAGAGCTA | 3′ pegRNA |
| | GAAATAGCAAGTTAAAATAAGGCTAGTCCGT | (Bxb1_38 nt_RTT2) |
| | TATCAACTTGAAAAAGTGGCACCGAGTCGGT | |
| | GCGGCTTGTCGACGACGGCGGTCTCCGTCGTC | |
| | AGGATCATATATCCAGAACCCT (SEQ ID NO: | |
| | 462) | |
| 463 | GTCAGGGTTCTGGATATCTGGTTTTAGAGCTA | 3′ pegRNA |
| | GAAATAGCAAGTTAAAATAAGGCTAGTCCGT | (Bxb1_38 nt_RTT2); contains |
| | TATCAACTTGAAAAAGTGGCACCGAGTCGGT | 3′ end modification TTTT |
| | GCGGCTTGTCGACGACGGCGGTCTCCGTCGTC | |
| | AGGATCATATATCCAGAACCTTTT (SEQ ID NO: | |
| | 463) | |
| 464 | GTCAGGGTTCTGGATATCTGGTTTTAGAGCTA | 3′ pegRNA |
| | GAAATAGCAAGTTAAAATAAGGCTAGTCCGT | (Bxb1_38 nt_RTT2); contains |
| | TATCAACTTGAAAAAGTGGCACCGAGTCGGT | 3′ end modification TTTT |
| | GCGGCTTGTCGACGACGGCGGTCTCCGTCGTC | |
| | AGGATCATATATCCAGAACCCTTTTT (SEQ ID | |
| | NO: 464) | |

TABLE 22

| Sequence | | |
|---|---|---|
| Number | Sequence | Description |
| 465 | GCAGACAGACTTGTCAC (SEQ ID NO: 465) | 3′ PEgRNA spacer (TGG |
| | | PAM) |
| 466 | GGCAGACAGACTTGTCAC (SEQ ID NO: 466) | 3′ PEgRNA spacer (TGG |
| | | PAM) |
| 467 | AGGCAGACAGACTTGTCAC (SEQ ID NO: 467) | 3′ PEgRNA spacer (TGG |
| | | PAM) |
| 468 | TAGGCAGACAGACTTGTCAC (SEQ ID NO: 468) | 3′ PEgRNA spacer (TGG |
| | | PAM) |
| 469 | ATAGGCAGACAGACTTGTCAC (SEQ ID NO: | 3′ PEgRNA spacer (TGG |
| | 469) | PAM) |
| 470 | AATAGGCAGACAGACTTGTCAC (SEQ ID NO: | 3′ PEgRNA spacer (TGG |
| | 470) | PAM) |
| 471 | ACAAG | 3′ PBS |
| 472 | ACAAGT | 3′ PBS |
| 473 | ACAAGTC | 3′ PBS |
| 474 | ACAAGTCT | 3′ PBS |
| 475 | ACAAGTCTG | 3′ PBS |
| 476 | ACAAGTCTGT (SEQ ID NO: 476) | 3′ PBS |
| 477 | ACAAGTCTGTC (SEQ ID NO: 477) | 3′ PBS |
| 478 | ACAAGTCTGTCT (SEQ ID NO: 478) | 3′ PBS |
| 479 | ACAAGTCTGTCTG (SEQ ID NO: 479) | 3′ PBS |
| 480 | ACAAGTCTGTCTGC (SEQ ID NO: 480) | 3′ PBS |
| 481 | ACAAGTCTGTCTGCC (SEQ ID NO: 481) | 3′ PBS |
| 482 | ACAAGTCTGTCTGCCT (SEQ ID NO: 482) | 3′ PBS |
| 483 | ACAAGTCTGTCTGCCTA (SEQ ID NO: 483) | 3′ PBS |
| 484 | ACAAGTCTGTCTGCCTAT (SEQ ID NO: 484) | 3′ PBS |
| 485 | ACAAGTCTGTCTGCCTATT (SEQ ID NO: 485) | 3′ PBS |
| 486 | TAGGCAGACAGACTTGTCACGTTTTAGAGCTA | 3′ pegRNA |
| | GAAATAGCAAGTTAAAATAAGGCTAGTCCGT | (Bxb1_38 nt_RTT2) |
| | TATCAACTTGAAAAAGTGGCACCGAGTCGGT | |
| | GCGGCTTGTCGACGACGGCGGTCTCCGTCGTC | |
| | AGGATCATACAAGTCTGT (SEQ ID NO: 486) | |
| 487 | TAGGCAGACAGACTTGTCACGTTTTAGAGCTA | 3′ pegRNA |
| | GAAATAGCAAGTTAAAATAAGGCTAGTCCGT | (Bxb1_38 nt_RTT2) |
| | TATCAACTTGAAAAAGTGGCACCGAGTCGGT | |
| | GCGGCTTGTCGACGACGGCGGTCTCCGTCGTC | |
| | AGGATCATACAAGTCTGTCT (SEQ ID NO: 487) | |
| 488 | TAGGCAGACAGACTTGTCACGTTTTAGAGCTA | 3′ pegRNA |
| | GAAATAGCAAGTTAAAATAAGGCTAGTCCGT | (Bxb1_38 nt_RTT2); contains |
| | TATCAACTTGAAAAAGTGGCACCGAGTCGGT | 3′ end modification TTTT |
| | GCGGCTTGTCGACGACGGCGGTCTCCGTCGTC | |
| | AGGATCATACAAGTCTGTTTTT (SEQ ID NO: | |
| | 488) | |
| 489 | TAGGCAGACAGACTTGTCACGTTTTAGAGCTA | 3′ pegRNA |
| | GAAATAGCAAGTTAAAATAAGGCTAGTCCGT | (Bxb1_38 nt_RTT2) |
| | TATCAACTTGAAAAAGTGGCACCGAGTCGGT | |
| | GCGGCTTGTCGACGACGGCGGTCTCCGTCGTC | |
| | AGGATCATACAAGTCTGTCTGC (SEQ ID NO: | |
| | 489) | |
| 490 | TAGGCAGACAGACTTGTCACGTTTTAGAGCTA | 3′ pegRNA |
| | GAAATAGCAAGTTAAAATAAGGCTAGTCCGT | (Bxb1_38 nt_RTT2); contains |
| | TATCAACTTGAAAAAGTGGCACCGAGTCGGT | 3′ end modification TTTT |
| | GCGGCTTGTCGACGACGGCGGTCTCCGTCGTC | |
| | AGGATCATACAAGTCTGTCTTTTT (SEQ ID NO: | |
| | 490) | |
| 491 | TAGGCAGACAGACTTGTCACGTTTTAGAGCTA | 3′ pegRNA |
| | GAAATAGCAAGTTAAAATAAGGCTAGTCCGT | (Bxb1_38 nt_RTT2); contains |
| | TATCAACTTGAAAAAGTGGCACCGAGTCGGT | 3′ end modification TTTT |
| | GCGGCTTGTCGACGACGGCGGTCTCCGTCGTC | |
| | AGGATCATACAAGTCTGTCTGCTTTT (SEQ ID | |
| | NO: 491) | |

TABLE 23

| Sequence | | |
|---|---|---|
| Number | Sequence | Description |
| 492 | GGGTTCTGGATATCTGT (SEQ ID NO: 492) | 3′ PEgRNA spacer (GGG |
| | | PAM) |
| 493 | AGGGTTCTGGATATCTGT (SEQ ID NO: 493) | 3′ PERNA spacer (GGG |
| | | PAM) |
| 494 | CAGGGTTCTGGATATCTGT (SEQ ID NO: 494) | 3′ PEgRNA spacer (GGG |
| | | PAM) |
| 495 | TCAGGGTTCTGGATATCTGT (SEQ ID NO: 495) | 3′ PEgRNA spacer (GGG |
| | | PAM) |
| 496 | GTCAGGGTTCTGGATATCTGT (SEQ ID NO: | 3′ PEgRNA spacer (GGG |
| | 496) | PAM) |
| 497 | GGTCAGGGTTCTGGATATCTGT (SEQ ID NO: | 3′ PEgRNA spacer (GGG |
| | 497) | PAM) |
| 498 | GATAT | 3′ PBS |
| 499 | GATATC | 3′ PBS |
| 500 | GATATCC | 3′ PBS |
| 501 | GATATCCA | 3′ PBS |
| 502 | GATATCCAG | 3′ PBS |
| 503 | GATATCCAGA (SEQ ID NO: 503) | 3′ PBS |
| 504 | GATATCCAGAA (SEQ ID NO: 504) | 3′ PBS |
| 505 | GATATCCAGAAC (SEQ ID NO: 505) | 3′ PBS |
| 506 | GATATCCAGAACC (SEQ ID NO: 506) | 3′ PBS |
| 507 | GATATCCAGAACCC (SEQ ID NO: 507) | 3′ PBS |
| 508 | GATATCCAGAACCCT (SEQ ID NO: 508) | 3′ PBS |
| 509 | GATATCCAGAACCCTG (SEQ ID NO: 509) | 3′ PBS |
| 510 | GATATCCAGAACCCTGA (SEQ ID NO: 510) | 3′ PBS |
| 511 | GATATCCAGAACCCTGAC (SEQ ID NO: 511) | 3′ PBS |
| 512 | GATATCCAGAACCCTGACC (SEQ ID NO: 512) | 3′ PBS |
| 513 | TCAGGGTTCTGGATATCTGTGTTTTAGAGCTA | 3′ pegRNA |
| | GAAATAGCAAGTTAAAATAAGGCTAGTCCGT | (Bxb1_38 nt_RTT2) |
| | TATCAACTTGAAAAAGTGGCACCGAGTCGGT | |
| | GCGGCTTGTCGACGACGGCGGTCTCCGTCGTC | |
| | AGGATCATGATATCCAGA (SEQ ID NO: 513) | |
| 514 | TCAGGGTTCTGGATATCTGTGTTTTAGAGCTA | 3′ pegRNA |
| | GAAATAGCAAGTTAAAATAAGGCTAGTCCGT | (Bxb1_38 nt_RTT2) |
| | TATCAACTTGAAAAAGTGGCACCGAGTCGGT | |
| | GCGGCTTGTCGACGACGGCGGTCTCCGTCGTC | |
| | AGGATCATGATATCCAGAAC (SEQ ID NO: 514) | |
| 515 | TCAGGGTTCTGGATATCTGTGTTTTAGAGCTA | 3′ pegRNA |
| | GAAATAGCAAGTTAAAATAAGGCTAGTCCGT | (Bxb1_38 nt_RTT2); contains |
| | TATCAACTTGAAAAAGTGGCACCGAGTCGGT | 3′ end modification TTTT |
| | GCGGCTTGTCGACGACGGCGGTCTCCGTCGTC | |
| | AGGATCATGATATCCAGATTTT (SEQ ID NO: | |
| | 515) | |
| 516 | TCAGGGTTCTGGATATCTGTGTTTTAGAGCTA | 3′ pegRNA |
| | GAAATAGCAAGTTAAAATAAGGCTAGTCCGT | (Bxb1_38 nt_RTT2) |
| | TATCAACTTGAAAAAGTGGCACCGAGTCGGT | |
| | GCGGCTTGTCGACGACGGCGGTCTCCGTCGTC | |
| | AGGATCATGATATCCAGAACCCT (SEQ ID NO: | |
| | 516) | |
| 517 | TCAGGGTTCTGGATATCTGTGTTTTAGAGCTA | 3′ pegRNA |
| | GAAATAGCAAGTTAAAATAAGGCTAGTCCGT | (Bxb1_38 nt_RTT2); contains |
| | TATCAACTTGAAAAAGTGGCACCGAGTCGGT | 3′ end modification TTTT |
| | GCGGCTTGTCGACGACGGCGGTCTCCGTCGTC | |
| | AGGATCATGATATCCAGAACTTTT (SEQ ID | |
| | NO: 517) | |
| 518 | TCAGGGTTCTGGATATCTGTGTTTTAGAGCTA | 3′ pegRNA |
| | GAAATAGCAAGTTAAAATAAGGCTAGTCCGT | (Bxb1_38 nt_RTT2); contains |
| | TATCAACTTGAAAAAGTGGCACCGAGTCGGT | 3′ end modification TTTT |
| | GCGGCTTGTCGACGACGGCGGTCTCCGTCGTC | |
| | AGGATCATGATATCCAGAACCCTTTTT (SEQ | |
| | ID NO: 518) | |

TABLE 24

| Sequence | | |
|---|---|---|
| Number | Sequence | Description |
| 519 | CTCAGCTGGTACACGGC (SEQ ID NO: 519) | 3′ PEgRNA spacer (AGG |
| | | PAM) |
| 520 | TCTCAGCTGGTACACGGC (SEQ ID NO: 520) | 3′ PEgRNA spacer (AGG |
| | | PAM) |
| 521 | CTCTCAGCTGGTACACGGC (SEQ ID NO: 521) | 3′ PEgRNA spacer (AGG |
| | | PAM) |
| 522 | TCTCTCAGCTGGTACACGGC (SEQ ID NO: 522) | 3′ PEgRNA spacer (AGG |
| | | PAM) |
| 523 | GTCTCTCAGCTGGTACACGGC (SEQ ID NO: | 3′ PEgRNA spacer (AGG |
| | 523) | PAM) |
| 524 | AGTCTCTCAGCTGGTACACGGC (SEQ ID NO: | 3′ PEgRNA spacer (AGG |
| | 524) | PAM) |
| 525 | GTGTA | 3′ PBS |
| 526 | GTGTAC | 3′ PBS |
| 527 | GTGTACC | 3′ PBS |
| 528 | GTGTACCA | 3′ PBS |
| 529 | GTGTACCAG | 3′ PBS |
| 530 | GTGTACCAGC (SEQ ID NO: 530) | 3′ PBS |
| 531 | GTGTACCAGCT (SEQ ID NO: 531) | 3′ PBS |
| 532 | GTGTACCAGCTG (SEQ ID NO: 532) | 3′ PBS |
| 533 | GTGTACCAGCTGA (SEQ ID NO: 533) | 3′ PBS |
| 534 | GTGTACCAGCTGAG (SEQ ID NO: 534) | 3′ PBS |
| 535 | GTGTACCAGCTGAGA (SEQ ID NO: 535) | 3′ PBS |
| 536 | GTGTACCAGCTGAGAG (SEQ ID NO: 536) | 3′ PBS |
| 537 | GTGTACCAGCTGAGAGA (SEQ ID NO: 537) | 3′ PBS |
| 538 | GTGTACCAGCTGAGAGAC (SEQ ID NO: 538) | 3′ PBS |
| 539 | GTGTACCAGCTGAGAGACT (SEQ ID NO: 539) | 3′ PBS |
| 540 | TCTCTCAGCTGGTACACGGCGTTTTAGAGCTA | 3′ pegRNA |
| | GAAATAGCAAGTTAAAATAAGGCTAGTCCGT | (Bxb1_30 nt_RTT2) |
| | TATCAACTTGAAAAAGTGGCACCGAGTCGGT | |
| | GCGACGACGGCGGTCTCCGTCGTCAGGATCAT | |
| | GTGTACCAGCT (SEQ ID NO: 540) | |
| 541 | TCTCTCAGCTGGTACACGGCGTTTTAGAGCTA | 3′ pegRNA |
| | GAAATAGCAAGTTAAAATAAGGCTAGTCCGT | (Bxb1_30 nt_RTT2) |
| | TATCAACTTGAAAAAGTGGCACCGAGTCGGT | |
| | GCGACGACGGCGGTCTCCGTCGTCAGGATCAT | |
| | GTGTACCAGCTGA (SEQ ID NO: 541) | |
| 542 | TCTCTCAGCTGGTACACGGCGTTTTAGAGCTA | 3′ pegRNA |
| | GAAATAGCAAGTTAAAATAAGGCTAGTCCGT | (Bxb1_30 nt_RTT1) |
| | TATCAACTTGAAAAAGTGGCACCGAGTCGGT | |
| | GCGACGACGGAGACCGCCGTCGTCGACAAGC | |
| | CGTGTACCAGCTGA (SEQ ID NO: 542) | |
| 543 | TCTCTCAGCTGGTACACGGCGTTTTAGAGCTA | 3′ pegRNA |
| | GAAATAGCAAGTTAAAATAAGGCTAGTCCGT | (Bxb1_30 nt_RTT2); contains |
| | TATCAACTTGAAAAAGTGGCACCGAGTCGGT | 3′ end modification TTTT |
| | GCGACGACGGCGGTCTCCGTCGTCAGGATCAT | |
| | GTGTACCAGCTTTTT (SEQ ID NO: 543) | |
| 544 | TCTCTCAGCTGGTACACGGCGTTTTAGAGCTA | 3′ pegRNA |
| | GAAATAGCAAGTTAAAATAAGGCTAGTCCGT | (Bxb1_30 nt_RTT2) |
| | TATCAACTTGAAAAAGTGGCACCGAGTCGGT | |
| | GCGACGACGGCGGTCTCCGTCGTCAGGATCAT | |
| | GTGTACCAGCTGAGA (SEQ ID NO: 544) | |
| 545 | TCTCTCAGCTGGTACACGGCGTTTTAGAGCTA | 3′ pegRNA |
| | GAAATAGCAAGTTAAAATAAGGCTAGTCCGT | (Bxb1_30 nt_RTT2); contains |
| | TATCAACTTGAAAAAGTGGCACCGAGTCGGT | 3′ end modification TTTT |
| | GCGACGACGGCGGTCTCCGTCGTCAGGATCAT | |
| | GTGTACCAGCTGATTTT (SEQ ID NO: 545) | |
| 546 | TCTCTCAGCTGGTACACGGCGTTTTAGAGCTA | 3′ pegRNA |
| | GAAATAGCAAGTTAAAATAAGGCTAGTCCGT | (Bxb1_30 nt_RTT1); contains |
| | TATCAACTTGAAAAAGTGGCACCGAGTCGGT | 3′ end modification TTTT |
| | GCGACGACGGAGACCGCCGTCGTCGACAAGC | |
| | CGTGTACCAGCTGATTTT (SEQ ID NO: 546) | |
| 547 | TCTCTCAGCTGGTACACGGCGTTTTAGAGCTA | 3′ pegRNA |
| | GAAATAGCAAGTTAAAATAAGGCTAGTCCGT | (Bxb1_38 nt_RTT2) |
| | TATCAACTTGAAAAAGTGGCACCGAGTCGGT | |
| | GCGGCTTGTCGACGACGGCGGTCTCCGTCGTC | |
| | AGGATCATGTGTACCAG (SEQ ID NO: 547) | |
| 548 | TCTCTCAGCTGGTACACGGCGTTTTAGAGCTA | 3′ pegRNA |
| | GAAATAGCAAGTTAAAATAAGGCTAGTCCGT | (Bxb1_30 nt_RTT2) |
| | TATCAACTTGAAAAAGTGGCACCGAGTCGGT | |
| | GCGACGACGGCGGTCTCCGTCGTCAGGATCAT | |
| | GTGTACCAGCTGAGAGA (SEQ ID NO: 548) | |
| 549 | TCTCTCAGCTGGTACACGGCGTTTTAGAGCTA | 3′ pegRNA |
| | GAAATAGCAAGTTAAAATAAGGCTAGTCCGT | (Bxb1_30 nt_RTT2); contains |
| | TATCAACTTGAAAAAGTGGCACCGAGTCGGT | 3′ end modification TTTT |
| | GCGACGACGGCGGTCTCCGTCGTCAGGATCAT | |
| | GTGTACCAGCTGAGATTTT (SEQ ID NO: 549) | |
| 550 | TCTCTCAGCTGGTACACGGCGTTTTAGAGCTA | 3′ pegRNA |
| | GAAATAGCAAGTTAAAATAAGGCTAGTCCGT | (Bxb1_38 nt_RTT2) |
| | TATCAACTTGAAAAAGTGGCACCGAGTCGGT | |
| | GCGGCTTGTCGACGACGGCGGTCTCCGTCGTC | |
| | AGGATCATGTGTACCAGCT (SEQ ID NO: 550) | |
| 551 | TCTCTCAGCTGGTACACGGCGTTTTAGAGCTA | 3′ pegRNA |
| | GAAATAGCAAGTTAAAATAAGGCTAGTCCGT | (Bxb1_38 nt_RTT2); contains |
| | TATCAACTTGAAAAAGTGGCACCGAGTCGGT | 3′ end modification TTTT |
| | GCGGCTTGTCGACGACGGCGGTCTCCGTCGTC | |
| | AGGATCATGTGTACCAGTTTT (SEQ ID NO: | |
| | 551) | |
| 552 | TCTCTCAGCTGGTACACGGCGTTTTAGAGCTA | 3′ pegRNA |
| | GAAATAGCAAGTTAAAATAAGGCTAGTCCGT | (Bxb1_30 nt_RTT2); contains |
| | TATCAACTTGAAAAAGTGGCACCGAGTCGGT | 3′ end modification TTTT |
| | GCGACGACGGCGGTCTCCGTCGTCAGGATCAT | |
| | GTGTACCAGCTGAGAGATTTT (SEQ ID NO: | |
| | 552) | |
| 553 | TCTCTCAGCTGGTACACGGCGTTTTAGAGCTA | 3′ pegRNA |
| | GAAATAGCAAGTTAAAATAAGGCTAGTCCGT | (Bxb1_30 nt_RTT2); contains |
| | TATCAACTTGAAAAAGTGGCACCGAGTCGGT | 3′ end modification TTTT |
| | GCGACGACGGCGGTCTCCGTCGTCAGGATCAT | |
| | GTGTACCAGCTGATTTTTTTT (SEQ ID NO: 553) | |
| 554 | TCTCTCAGCTGGTACACGGCGTTTTAGAGCTA | 3′ pegRNA |
| | GAAATAGCAAGTTAAAATAAGGCTAGTCCGT | (Bxb1_38 nt_RTT2) |
| | TATCAACTTGAAAAAGTGGCACCGAGTCGGT | |
| | GCGGCTTGTCGACGACGGCGGTCTCCGTCGTC | |
| | AGGATCATGTGTACCAGCTGA (SEQ ID NO: | |
| | 554) | |
| 555 | TCTCTCAGCTGGTACACGGCGTTTTAGAGCTA | 3′ pegRNA |
| | GAAATAGCAAGTTAAAATAAGGCTAGTCCGT | (Bxb1_38 nt_RTT1) |
| | TATCAACTTGAAAAAGTGGCACCGAGTCGGT | |
| | GCATGATCCTGACGACGGAGACCGCCGTCGT | |
| | CGACAAGCCGTGTACCAGCTGA (SEQ ID NO: | |
| | 555) | |
| 556 | TCTCTCAGCTGGTACACGGCGTTTTAGAGCTA | 3′ pegRNA |
| | GAAATAGCAAGTTAAAATAAGGCTAGTCCGT | (Bxb1_38 nt_RTT2); contains |
| | TATCAACTTGAAAAAGTGGCACCGAGTCGGT | 3′ end modification TTTT |
| | GCGGCTTGTCGACGACGGCGGTCTCCGTCGTC | |
| | AGGATCATGTGTACCAGCTTTTT (SEQ ID NO: | |
| | 556) | |
| 557 | TCTCTCAGCTGGTACACGGCGTTTTAGAGCTA | 3′ pegRNA |
| | GAAATAGCAAGTTAAAATAAGGCTAGTCCGT | (Bxb1_38 nt_RTT2) |
| | TATCAACTTGAAAAAGTGGCACCGAGTCGGT | |
| | GCGGCTTGTCGACGACGGCGGTCTCCGTCGTC | |
| | AGGATCATGTGTACCAGCTGAGA (SEQ ID NO: | |
| | 557) | |
| 558 | TCTCTCAGCTGGTACACGGCGTTTTAGAGCTA | 3′ pegRNA |
| | GAAATAGCAAGTTAAAATAAGGCTAGTCCGT | (Bxb1_38 nt_RTT2); contains |
| | TATCAACTTGAAAAAGTGGCACCGAGTCGGT | 3′ end modification TTTT |
| | GCGGCTTGTCGACGACGGCGGTCTCCGTCGTC | |
| | AGGATCATGTGTACCAGCTGATTTT (SEQ ID | |
| | NO: 558) | |
| 559 | TCTCTCAGCTGGTACACGGCGTTTTAGAGCTA | 3′ pegRNA |
| | GAAATAGCAAGTTAAAATAAGGCTAGTCCGT | (Bxb1_38 nt_RTT1); contains |
| | TATCAACTTGAAAAAGTGGCACCGAGTCGGT | 3′ end modification TTTT |
| | GCATGATCCTGACGACGGAGACCGCCGTCGT | |
| | CGACAAGCCGTGTACCAGCTGATTTT (SEQ ID | |
| | NO: 559) | |
| 560 | TCTCTCAGCTGGTACACGGCGTTTTAGAGCTA | 3′ pegRNA |
| | GAAATAGCAAGTTAAAATAAGGCTAGTCCGT | (Bxb1_38 nt_RTT2) |
| | TATCAACTTGAAAAAGTGGCACCGAGTCGGT | |
| | GCGGCTTGTCGACGACGGCGGTCTCCGTCGTC | |
| | AGGATCATGTGTACCAGCTGAGAGA (SEQ ID | |
| | NO: 560) | |
| 561 | TCTCTCAGCTGGTACACGGCGTTTTAGAGCTA | 3′ pegRNA |
| | GAAATAGCAAGTTAAAATAAGGCTAGTCCGT | (Bxb1_38 nt_RTT2); contains |
| | TATCAACTTGAAAAAGTGGCACCGAGTCGGT | 3′ end modification TTTT |
| | GCGGCTTGTCGACGACGGCGGTCTCCGTCGTC | |
| | AGGATCATGTGTACCAGCTGAGATTTT (SEQ | |
| | ID NO: 561) | |
| 562 | TCTCTCAGCTGGTACACGGCGTTTTAGAGCTA | 3′ pegRNA |
| | GAAATAGCAAGTTAAAATAAGGCTAGTCCGT | (Bxb1_38 nt_RTT2); contains |
| | TATCAACTTGAAAAAGTGGCACCGAGTCGGT | 3′ end modification TTTT |
| | GCGGCTTGTCGACGACGGCGGTCTCCGTCGTC | |
| | AGGATCATGTGTACCAGCTGAGAGATTTT | |
| | (SEQ ID NO: 562) | |
| 1135 | TCTCTCAGCTGGTACACGGCGTTTTAGAGCTA | 3′ pegRNA |
| | GAAATAGCAAGTTAAAATAAGGCTAGTCCGT | (Bxb1_30 nt_RTT1); contains |
| | TATCAACTTGAAAAAGTGGCACCGAGTCGGT | 3′ end modification TTTT |
| | GCGACGACGGAGACCGCCGTCGTCGACAAGC | |
| | CGTGTACCAGCTTTTT (SEQ ID NO: 1135) | |
| 1142 | TCTCTCAGCTGGTACACGGCGTTTTAGAGCTA | 3′ pegRNA |
| | GAAATAGCAAGTTAAAATAAGGCTAGTCCGT | (Bxb1_29 nt_RTT2); contains |
| | TATCAACTTGAAAAAGTGGCACCGAGTCGGT | 3′ end modification TTTT |
| | GCACGACGGCGGTCTCCGTCGTCAGGATCATG | |
| | TGTACCAGCTGATTTT (SEQ ID NO: 1142) | |

TABLE 25

| Sequence | | |
|---|---|---|
| Number | Sequence | Description |
| 563 | GTGGGACAAGAGGATCA (SEQ ID NO: 563) | 3′ PEgRNA spacer (GGG |
| | | PAM) |
| 564 | TGTGGGACAAGAGGATCA (SEQ ID NO: 564) | 3′ PEgRNA spacer (GGG |
| | | PAM) |
| 565 | CTGTGGGACAAGAGGATCA (SEQ ID NO: 565) | 3′ PERNA spacer (GGG |
| | | PAM) |
| 566 | TCTGTGGGACAAGAGGATCA (SEQ ID NO: 566) | 3′ PERNA spacer (GGG |
| | | PAM) |
| 567 | ATCTGTGGGACAAGAGGATCA (SEQ ID NO: | 3′ PEgRNA spacer (GGG |
| | 567) | PAM) |
| 568 | TATCTGTGGGACAAGAGGATCA (SEQ ID NO: | 3′ PEgRNA spacer (GGG |
| | 568) | PAM) |
| 569 | TCCTC | 3′ PBS |
| 570 | TCCTCT | 3′ PBS |
| 571 | TCCTCTT | 3′ PBS |
| 572 | TCCTCTTG | 3′ PBS |
| 573 | TCCTCTTGT | 3′ PBS |
| 574 | TCCTCTTGTC (SEQ ID NO: 574) | 3′ PBS |
| 575 | TCCTCTTGTCC (SEQ ID NO: 575) | 3′ PBS |
| 576 | TCCTCTTGTCCC (SEQ ID NO: 576) | 3′ PBS |
| 577 | TCCTCTTGTCCCA (SEQ ID NO: 577) | 3′ PBS |
| 578 | TCCTCTTGTCCCAC (SEQ ID NO: 578) | 3′ PBS |
| 579 | TCCTCTTGTCCCACA (SEQ ID NO: 579) | 3′ PBS |
| 580 | TCCTCTTGTCCCACAG (SEQ ID NO: 580) | 3′ PBS |
| 581 | TCCTCTTGTCCCACAGA (SEQ ID NO: 581) | 3′ PBS |
| 582 | TCCTCTTGTCCCACAGAT (SEQ ID NO: 582) | 3′ PBS |
| 583 | TCCTCTTGTCCCACAGATA (SEQ ID NO: 583) | 3′ PBS |
| 584 | TCTGTGGGACAAGAGGATCAGTTTTAGAGCTA | 3′ pegRNA |
| | GAAATAGCAAGTTAAAATAAGGCTAGTCCGT | (Bxb1_38 nt_RTT2) |
| | TATCAACTTGAAAAAGTGGCACCGAGTCGGT | |
| | GCGGCTTGTCGACGACGGCGGTCTCCGTCGTC | |
| | AGGATCATTCCTCTTGT (SEQ ID NO: 584) | |
| 585 | TCTGTGGGACAAGAGGATCAGTTTTAGAGCTA | 3′ pegRNA |
| | GAAATAGCAAGTTAAAATAAGGCTAGTCCGT | (Bxb1_38 nt_RTT2) |
| | TATCAACTTGAAAAAGTGGCACCGAGTCGGT | |
| | GCGGCTTGTCGACGACGGCGGTCTCCGTCGTC | |
| | AGGATCATTCCTCTTGTCC (SEQ ID NO: 585) | |
| 586 | TCTGTGGGACAAGAGGATCAGTTTTAGAGCTA | 3′ pegRNA |
| | GAAATAGCAAGTTAAAATAAGGCTAGTCCGT | (Bxb1_38 nt_RTT2); contains |
| | TATCAACTTGAAAAAGTGGCACCGAGTCGGT | 3′ end modification TTTT |
| | GCGGCTTGTCGACGACGGCGGTCTCCGTCGTC | |
| | AGGATCATTCCTCTTGTTTTT (SEQ ID NO: 586) | |
| 587 | TCTGTGGGACAAGAGGATCAGTTTTAGAGCTA | 3′ pegRNA |
| | GAAATAGCAAGTTAAAATAAGGCTAGTCCGT | (Bxb1_38 nt_RTT2) |
| | TATCAACTTGAAAAAGTGGCACCGAGTCGGT | |
| | GCGGCTTGTCGACGACGGCGGTCTCCGTCGTC | |
| | AGGATCATTCCTCTTGTCCCA (SEQ ID NO: 587) | |
| 588 | TCTGTGGGACAAGAGGATCAGTTTTAGAGCTA | 3′ pegRNA |
| | GAAATAGCAAGTTAAAATAAGGCTAGTCCGT | (Bxb1_38 nt_RTT2); contains |
| | TATCAACTTGAAAAAGTGGCACCGAGTCGGT | 3′ end modification TTTT |
| | GCGGCTTGTCGACGACGGCGGTCTCCGTCGTC | |
| | AGGATCATTCCTCTTGTCCTTTT (SEQ ID NO: | |
| | 588) | |
| 589 | TCTGTGGGACAAGAGGATCAGTTTTAGAGCTA | 3′ pegRNA |
| | GAAATAGCAAGTTAAAATAAGGCTAGTCCGT | (Bxb1_38 nt_RTT2); contains |
| | TATCAACTTGAAAAAGTGGCACCGAGTCGGT | 3′ end modification TTTT |
| | GCGGCTTGTCGACGACGGCGGTCTCCGTCGTC | |
| | AGGATCATTCCTCTTGTCCCATTTT (SEQ ID | |
| | NO: 589) | |

TABLE 26

| Sequence | | |
|---|---|---|
| Number | Sequence | Description |
| 1060 | ACTCACGCTGGATAGCCTCC (SEQ ID NO: | spacer |
| | 1060) | |
| 1061 | CTCACGCTGGATAGCCTCC (SEQ ID NO: | spacer |
| | 1061) | |
| 1062 | TCACGCTGGATAGCCTCC (SEQ ID NO: | spacer |
| | 1062) | |
| 1063 | CACGCTGGATAGCCTCC (SEQ ID NO: | spacer |
| | 1063) | |
| 1064 | GGCTA | PBS |
| 1065 | GGCTAT | PBS |
| 1066 | GGCTATC | PBS |
| 1067 | GGCTATCC | PBS |
| 1068 | GGCTATCCA | PBS |
| 1069 | GGCTATCCAG (SEQ ID NO: 1069) | PBS |
| 1070 | GGCTATCCAGC (SEQ ID NO: 1070) | PBS |
| 1071 | GGCTATCCAGCG (SEQ ID NO: 1071) | PBS |
| 1072 | GGCTATCCAGCGT (SEQ ID NO: 1072) | PBS |
| 1073 | GGCTATCCAGCGTG (SEQ ID NO: 1073) | PBS |
| 1074 | GGCTATCCAGCGTGA (SEQ ID NO: 1074) | PBS |
| 1075 | GGCTATCCAGCGTGAG (SEQ ID NO: 1075) | PBS |
| 1076 | GGCTATCCAGCGTGAGT (SEQ ID NO: | PBS |
| | 1076) | |
| 1077 | CTGGCCTGTAATAAGA (SEQ ID NO: 1077) | RTT (c.54InsTAATAA |
| | | edit) |
| 1078 | CTTTCTGGCCTGTAATAAGA (SEQ ID NO: | RTT (c.54InsTAATAA |
| | 1078) | edit) |
| 1079 | CTCTCTTTCTGGCCTGTAATAAGA (SEQ ID | RTT (c.54InsTAATAA |
| | NO: 1079) | edit) |
| 1080 | TCTCTTTCTGGCTGGA (SEQ ID NO: 1080) | RTT (c.51delC edit)*1 |
| 1081 | TCTGGCTGGA (SEQ ID NO: 1081) | RTT (c.51delC edit)*1 |
| 1082 | TTTCTGGCTGGA (SEQ ID NO: 1082) | RTT (c.51delC edit)*1 |
| 1083 | TCTTTCTGGCTGGA (SEQ ID NO: 1083) | RTT (c.51delC edit)*1 |
| 1084 | TCTCTCTTTCTGGCTGGA (SEQ ID NO: | RTT (c.51delC edit)*1 |
| | 1084) | |
| 1085 | ACTCTCTCTTTCTGGCTGGA (SEQ ID NO: | RTT (c.51delC edit)*1 |
| | 1085) | |
| 1086 | CTGGGCCTGGA (SEQ ID NO: 1086) | RTT (c.50insG edit)*2 |
| 1087 | TTCTGGGCCTGGA (SEQ ID NO: 1087) | RTT (c.50insG edit)*2 |
| 1088 | CTTTCTGGGCCTGGA (SEQ ID NO: 1088) | RTT (c.50insG edit)*2 |
| 1089 | CTCTTTCTGGGCCTGGA (SEQ ID NO: 1089) | RTT (c.50insG edit)*2 |
| 1090 | ACTCACGCTGGATAGCCTCCGTTTTAGAG | PEgRNA (c.51delC edit, |
| | CTAGAAATAGCAAGTTAAAATAAGGCTA | contains gRNA core SEQ |
| | GTCCGTTATCAACTTGAAAAAGTGGCACC | ID NO: 590)*1, contains 3′ |
| | GAGTCGGTGCTCTCTTTCTGGCTGGAGGC | end modification TTTT |
| | TATCCAGCGTTTT (SEQ ID NO: 1090) | |
| 1091 | ACTCACGCTGGATAGCCTCCGTTTTAGAG | PEgRNA (c.51delC edit, |
| | CTAGAAATAGCAAGTTAAAATAAGGCTA | contains gRNA core SEQ |
| | GTCCGTTATCAACTTGAAAAAGTGGCACC | ID NO: 590)*1, contains 3′ |
| | GAGTCGGTGCTCTGGCTGGAGGCTATCCA | end modification TTTT |
| | GCGTTTT (SEQ ID NO: 1091) | |
| 1092 | ACTCACGCTGGATAGCCTCCGTTTTAGAG | PEgRNA (c.51delC edit, |
| | CTAGAAATAGCAAGTTAAAATAAGGCTA | contains gRNA core SEQ |
| | GTCCGTTATCAACTTGAAAAAGTGGCACC | ID NO: 590)*1, contains 3′ |
| | GAGTCGGTGCTTTCTGGCTGGAGGCTATC | end modification TTTT |
| | CAGCGTTTT (SEQ ID NO: 1092) | |
| 1093 | ACTCACGCTGGATAGCCTCCGTTTTAGAG | PEgRNA (c.51delC edit, |
| | CTAGAAATAGCAAGTTAAAATAAGGCTA | contains gRNA core SEQ |
| | GTCCGTTATCAACTTGAAAAAGTGGCACC | ID NO: 590)*1, contains 3′ |
| | GAGTCGGTGCTCTTTCTGGCTGGAGGCTA | end modification TTTT |
| | TCCAGCGTTTT (SEQ ID NO: 1093) | |
| 1094 | ACTCACGCTGGATAGCCTCCGTTTTAGAG | PEgRNA (c.51delC edit, |
| | CTAGAAATAGCAAGTTAAAATAAGGCTA | contains gRNA core SEQ |
| | GTCCGTTATCAACTTGAAAAAGTGGCACC | ID NO: 590)*1, contains 3′ |
| | GAGTCGGTGCTCTCTCTTTCTGGCTGGAG | end modification TTTT |
| | GCTATCCAGCGTTTT (SEQ ID NO: 1094) | |
| 1095 | ACTCACGCTGGATAGCCTCCGTTTTAGAG | PEgRNA (c.51delC edit, |
| | CTAGAAATAGCAAGTTAAAATAAGGCTA | contains gRNA core SEQ |
| | GTCCGTTATCAACTTGAAAAAGTGGCACC | ID NO: 590)*1, contains 3′ |
| | GAGTCGGTGCACTCTCTCTTTCTGGCTGG | end modification TTTT |
| | AGGCTATCCAGCGTTTT (SEQ ID NO: 1095) | |
| 1096 | ACTCACGCTGGATAGCCTCCGTTTTAGAG | PEgRNA (c.50insG edit, |
| | CTAGAAATAGCAAGTTAAAATAAGGCTA | contains gRNA core SEQ |
| | GTCCGTTATCAACTTGAAAAAGTGGCACC | ID NO: 590)*2, contains 3′ |
| | GAGTCGGTGCCTTTCTGGGCCTGGAGGCT | end modification TTTT |
| | ATCCAGCGTTTT (SEQ ID NO: 1096) | |
| 1097 | ACTCACGCTGGATAGCCTCCGTTTTAGAG | PEgRNA (c.50insG edit, |
| | CTAGAAATAGCAAGTTAAAATAAGGCTA | contains gRNA core SEQ |
| | GTCCGTTATCAACTTGAAAAAGTGGCACC | ID NO: 590)*2, contains 3′ |
| | GAGTCGGTGCCTGGGCCTGGAGGCTATCC | end modification TTTT |
| | TTTT (SEQ ID NO: 1097) | |
| 1098 | ACTCACGCTGGATAGCCTCCGTTTTAGAG | PERNA (c.50insG edit, |
| | CTAGAAATAGCAAGTTAAAATAAGGCTA | contains gRNA core SEQ |
| | GTCCGTTATCAACTTGAAAAAGTGGCACC | ID NO: 590)*2, contains 3′ |
| | GAGTCGGTGCTTCTGGGCCTGGAGGCTAT | end modification TTTT |
| | CCTTTT (SEQ ID NO: 1098) | |
| 1099 | ACTCACGCTGGATAGCCTCCGTTTTAGAG | PEgRNA (c.50insG edit, |
| | CTAGAAATAGCAAGTTAAAATAAGGCTA | contains gRNA core SEQ |
| | GTCCGTTATCAACTTGAAAAAGTGGCACC | ID NO: 590)*2, contains 3′ |
| | GAGTCGGTGCCTTTCTGGGCCTGGAGGCT | end modification TTTT |
| | ATCCTTTT (SEQ ID NO: 1099) | |
| 1100 | ACTCACGCTGGATAGCCTCCGTTTTAGAG | PEgRNA (c.50insG edit, |
| | CTAGAAATAGCAAGTTAAAATAAGGCTA | contains gRNA core SEQ |
| | GTCCGTTATCAACTTGAAAAAGTGGCACC | ID NO: 590)*2, contains 3′ |
| | GAGTCGGTGCCTCTTTCTGGGCCTGGAGG | end modification TTTT |
| | CTATCCTTTT (SEQ ID NO: 1100) | |
| 1101 | ACTCACGCTGGATAGCCTCCGTTTTAGAG | PEgRNA (c.50insG edit, |
| | CTAGAAATAGCAAGTTAAAATAAGGCTA | contains gRNA core SEQ |
| | GTCCGTTATCAACTTGAAAAAGTGGCACC | ID NO: 590)*2, contains 3′ |
| | GAGTCGGTGCCTGGGCCTGGAGGCTATCC | end modification TTTT |
| | AGTTTT (SEQ ID NO: 1101) | |
| 1102 | ACTCACGCTGGATAGCCTCCGTTTTAGAG | PEgRNA (c.50insG edit, |
| | CTAGAAATAGCAAGTTAAAATAAGGCTA | contains gRNA core SEQ |
| | GTCCGTTATCAACTTGAAAAAGTGGCACC | ID NO: 590)*2, contains 3′ |
| | GAGTCGGTGCTTCTGGGCCTGGAGGCTAT | end modification TTTT |
| | CCAGTTTT (SEQ ID NO: 1102) | |
| 1103 | ACTCACGCTGGATAGCCTCCGTTTTAGAG | PEgRNA (c.50insG edit, |
| | CTAGAAATAGCAAGTTAAAATAAGGCTA | contains gRNA core SEQ |
| | GTCCGTTATCAACTTGAAAAAGTGGCACC | ID NO: 590)*2, contains 3′ |
| | GAGTCGGTGCCTTTCTGGGCCTGGAGGCT | end modification TTTT |
| | ATCCAGTTTT (SEQ ID NO: 1103) | |
| 1104 | ACTCACGCTGGATAGCCTCCGTTTTAGAG | PEgRNA (c.50insG edit, |
| | CTAGAAATAGCAAGTTAAAATAAGGCTA | contains gRNA core SEQ |
| | GTCCGTTATCAACTTGAAAAAGTGGCACC | ID NO: 590)*2, contains 3′ |
| | GAGTCGGTGCCTCTTTCTGGGCCTGGAGG | end modification TTTT |
| | CTATCCAGTTTT (SEQ ID NO: 1104) | |
| 1105 | ACTCACGCTGGATAGCCTCCGTTTTAGAG | PEgRNA (c.50insG edit, |
| | CTAGAAATAGCAAGTTAAAATAAGGCTA | contains gRNA core SEQ |
| | GTCCGTTATCAACTTGAAAAAGTGGCACC | ID NO: 590)*2, contains 3′ |
| | GAGTCGGTGCCTGGGCCTGGAGGCTATCC | end modification TTTT |
| | AGCGTTTT (SEQ ID NO: 1105) | |
| 1106 | ACTCACGCTGGATAGCCTCCGTTTTAGAG | PEgRNA (c.50insG edit, |
| | CTAGAAATAGCAAGTTAAAATAAGGCTA | contains gRNA core SEQ |
| | GTCCGTTATCAACTTGAAAAAGTGGCACC | ID NO: 590)*2, contains 3′ |
| | GAGTCGGTGCTTCTGGGCCTGGAGGCTAT | end modification TTTT |
| | CCAGCGTTTT (SEQ ID NO: 1106) | |
| 1107 | ACTCACGCTGGATAGCCTCCGTTTTAGAG | PEgRNA (c.50insG edit, |
| | CTAGAAATAGCAAGTTAAAATAAGGCTA | contains gRNA core SEQ |
| | GTCCGTTATCAACTTGAAAAAGTGGCACC | ID NO: 590)*2, contains 3′ |
| | GAGTCGGTGCCTCTTTCTGGGCCTGGAGG | end modification TTTT |
| | CTATCCAGCGTTTT (SEQ ID NO: 1107) | |
| 1108 | ACTCACGCTGGATAGCCTCCGTTTTAGAG | PEgRNA (c.50insG edit, |
| | CTAGAAATAGCAAGTTAAAATAAGGCTA | contains gRNA core SEQ |
| | GTCCGTTATCAACTTGAAAAAGTGGCACC | ID NO: 590)*2, contains 3′ |
| | GAGTCGGTGCCTGGGCCTGGAGGCTATCC | end modification TTTT |
| | AGCGTGTTTT (SEQ ID NO: 1108) | |
| 1109 | ACTCACGCTGGATAGCCTCCGTTTTAGAG | PEgRNA (c.50insG edit, |
| | CTAGAAATAGCAAGTTAAAATAAGGCTA | contains gRNA core SEQ |
| | GTCCGTTATCAACTTGAAAAAGTGGCACC | ID NO: 590)*2, contains 3′ |
| | GAGTCGGTGCTTCTGGGCCTGGAGGCTAT | end modification TTTT |
| | CCAGCGTGTTTT (SEQ ID NO: 1109) | |
| 1110 | ACTCACGCTGGATAGCCTCCGTTTTAGAG | PERNA (c.50insG edit, |
| | CTAGAAATAGCAAGTTAAAATAAGGCTA | contains gRNA core SEQ |
| | GTCCGTTATCAACTTGAAAAAGTGGCACC | ID NO: 590)*2, contains 3′ |
| | GAGTCGGTGCCTTTCTGGGCCTGGAGGCT | end modification TTTT |
| | ATCCAGCGTGTTTT (SEQ ID NO: 1110) | |
| 1111 | ACTCACGCTGGATAGCCTCCGTTTTAGAG | PEgRNA (c.50insG edit, |
| | CTAGAAATAGCAAGTTAAAATAAGGCTA | contains gRNA core SEQ |
| | GTCCGTTATCAACTTGAAAAAGTGGCACC | ID NO: 590)*2, contains 3′ |
| | GAGTCGGTGCCTCTTTCTGGGCCTGGAGG | end modification TTTT |
| | CTATCCAGCGTGTTTT (SEQ ID NO: 1111) | |
| 1112 | ACTCACGCTGGATAGCCTCCGTTTAAGAG | PEgRNA |
| | CTAGAAATAGCAAGTTTAAATAAGGCTAG | (c.54insTAATAA edit, |
| | TCCGTTATCAGCGTGAAAACGCGGCACCG | contains gRNA core SEQ |
| | AGTCGGTGCCTGGCCTGTAATAAGAGGCT | ID NO: 604) |
| | ATCCA (SEQ ID NO: 1112) | |
| 1113 | ACTCACGCTGGATAGCCTCCGTTTAAGAG | PEgRNA |
| | CTAGAAATAGCAAGTTTAAATAAGGCTAG | (c.54insTAATAA edit, |
| | TCCGTTATCAGCGTGAAAACGCGGCACCG | contains gRNA core SEQ |
| | AGTCGGTGCCTTTCTGGCCTGTAATAAGA | ID NO: 604) |
| | GGCTATCCA (SEQ ID NO: 1113) | |
| 1114 | ACTCACGCTGGATAGCCTCCGTTTAAGAG | PEgRNA |
| | CTAGAAATAGCAAGTTTAAATAAGGCTAG | (c.54insTAATAA edit, |
| | TCCGTTATCAGCGTGAAAACGCGGCACCG | contains gRNA core SEQ |
| | AGTCGGTGCCTCTCTTTCTGGCCTGTAATA | ID NO: 604) |
| | AGAGGCTATCCA (SEQ ID NO: 1114) | |
| 1115 | ACTCACGCTGGATAGCCTCCGTTTAAGAG | PEgRNA |
| | CTAGAAATAGCAAGTTTAAATAAGGCTAG | (c.54insTAATAA edit, |
| | TCCGTTATCAGCGTGAAAACGCGGCACCG | contains gRNA core SEQ |
| | AGTCGGTGCCTGGCCTGTAATAAGAGGCT | ID NO: 604) |
| | ATCCAGCG (SEQ ID NO: 1115) | |
| 1116 | ACTCACGCTGGATAGCCTCCGTTTAAGAG | PEgRNA |
| | CTAGAAATAGCAAGTTTAAATAAGGCTAG | (c.54insTAATAA edit, |
| | TCCGTTATCAGCGTGAAAACGCGGCACCG | contains gRNA core SEQ |
| | AGTCGGTGCCTTTCTGGCCTGTAATAAGA | ID NO: 604) |
| | GGCTATCCAGCG (SEQ ID NO: 1116) | |
| 1117 | ACTCACGCTGGATAGCCTCCGTTTAAGAG | PEgRNA |
| | CTAGAAATAGCAAGTTTAAATAAGGCTAG | (c.54insTAATAA edit, |
| | TCCGTTATCAGCGTGAAAACGCGGCACCG | contains gRNA core SEQ |
| | AGTCGGTGCCTCTCTTTCTGGCCTGTAATA | ID NO: 604) |
| | AGAGGCTATCCAGCG (SEQ ID NO: 1117) | |
| 1118 | ACTCACGCTGGATAGCCTCCGTTTAAGAG | PEgRNA |
| | CTAGAAATAGCAAGTTTAAATAAGGCTAG | (c.54insTAATAA edit, |
| | TCCGTTATCAGCGTGAAAACGCGGCACCG | contains gRNA core SEQ |
| | AGTCGGTGCCTGGCCTGTAATAAGAGGCT | ID NO: 604) |
| | ATCCAGCGTGA (SEQ ID NO: 1118) | |
| 1119 | ACTCACGCTGGATAGCCTCCGTTTAAGAG | PEgRNA |
| | CTAGAAATAGCAAGTTTAAATAAGGCTAG | (c.54insTAATAA edit, |
| | TCCGTTATCAGCGTGAAAACGCGGCACCG | contains gRNA core SEQ |
| | AGTCGGTGCCTTTCTGGCCTGTAATAAGA | ID NO: 604) |
| | GGCTATCCAGCGTGA (SEQ ID NO: 1119) | |
| 1120 | ACTCACGCTGGATAGCCTCCGTTTAAGAG | PEgRNA |
| | CTAGAAATAGCAAGTTTAAATAAGGCTAG | (c.54insTAATAA edit, |
| | TCCGTTATCAGCGTGAAAACGCGGCACCG | contains gRNA core SEQ |
| | AGTCGGTGCCTCTCTTTCTGGCCTGTAATA | ID NO: 604) |
| | AGAGGCTATCCAGCGTGA (SEQ ID NO: | |
| | 1120) | |
| 1121 | GGCCGAGATGTCTCGCTCCG (SEQ ID NO: | ngRNA spacer |
| | 1121) | |
| 1122 | TCCTGAAGCTGACAGCATTC (SEQ ID NO: | ngRNA spacer |
| | 1122) | |
| 1123 | TTCCTGAAGCTGACAGCATT (SEQ ID NO: | ngRNA spacer |
| | 1123) | |
| 1124 | TACTCTCTCTTTCTGGCTGG (SEQ ID NO: | ngRNA spacer*1 |
| | 1124) | |
| 1125 | CTCTCTCTTTCTGGGCCTGG (SEQ ID NO: | ngRNA spacer*2 |
| | 1125) | |
| 1126 | CTACTCTCTCTTTCTGGGC (SEQ ID NO: | ngRNA spacer*2 |
| | 1126) | |
| 1129 | CTACTCTCTCTTTCTGGGCGTTTTAGAGCT | ngRNA (contains ngRNA |
| | AGAAATAGCAAGTTAAAATAAGGCTAGT | spacer SEQ ID NO: 1126 |
| | CCGTTATCAACTTGAAAAAGTGGCACCGA | and gRNA core SEQ ID |
| | GTCGGTGC (SEQ ID NO: 1129) | NO: 590) |
| 1130 | CTACTCTCTCTTTCTGGGCGTTTTAGAGCT | ngRNA (contains ngRNA |
| | AGAAATAGCAAGTTAAAATAAGGCTAGT | spacer SEQ ID NO: 1126 |
| | CCGTTATCAACTTGAAAAAGTGGCACCGA | and gRNA core SEQ ID |
| | GTCGGTGCTTTT (SEQ ID NO: 1130) | NO: 590) |

### Prime Editing Compositions

Disclosed herein, in some embodiments, are compositions, systems, and methods using a prime editing composition. The term “prime editing composition” or “prime editing system” refers to compositions involved in the method of prime editing as described herein. A prime editing composition may include a prime editor, e.g., a prime editor fusion protein, and a PEgRNA. A prime editing composition may further comprise additional elements. Components of a prime editing composition may be combined to form a complex for prime editing, or may be kept separately, e.g., for administration purposes.

In some embodiments, a prime editing composition comprises a first prime editing guide RNA (PEgRNA), a second PEgRNA, and a prime editor. In some embodiments, a prime editing composition comprises a first prime editing guide RNA (PEgRNA), a second PEgRNA, and a prime editor fusion protein complexed with the first PEgRNA and a prime editor fusion protein complexed with the second PEgRNA. In some embodiments, the prime editor fusion protein complexed with the first PEgRNA and the prime editor fusion protein complexed with the second PEgRNA are the identical prime editor fusion protein. In some embodiments, the prime editor fusion protein complexed with the first PEgRNA and the prime editor fusion protein complexed with the second PEgRNA are different prime editor fusion proteins.

In some embodiments, a prime editing composition comprises a first prime editing guide RNA (PERNA), a second PEgRNA, and a prime editor comprising a DNA binding domain and a DNA polymerase domain associated with each other through the first PEgRNA and/or the second PEgRNA. For example, the prime editing composition may comprise a prime editor comprising a DNA binding domain and a DNA polymerase domain linked to each other by an RNA-protein recruitment aptamer RNA sequence, which is linked to either or both of the first and second PEgRNAs. In some embodiments, the prime editing composition comprises a first PEgRNA, a second PEgRNA, and a prime editor comprising a DNA binding domain and a DNA polymerase domain, wherein the prime editor for both the first PEgRNA and the second PEgRNA are the same. In some embodiments, the prime editing composition comprises a first PEgRNA, a second PERNA, and a prime editor comprising a DNA binding domain and a DNA polymerase domain, wherein the prime editor for both the first PERNA and the second PERNA are different.

In some embodiments, a prime editing composition comprises a first PEgRNA, a second PEgRNA, and a polynucleotide, a polynucleotide construct, or a vector that encodes a prime editor fusion protein. In some embodiments, a prime editing composition comprises a first PERNA, a second PERNA, and one or more polynucleotides, one or more polynucleotide constructs, or one or more vectors that encode a prime editor comprising a DNA binding domain and a DNA polymerase domain. In some embodiments, a prime editing composition comprises a first PEgRNA, a second PEgRNA, and a polynucleotide, a polynucleotide construct, or a vector that encodes a prime editor fusion protein. In some embodiments, a prime editing composition comprises multiple polynucleotides, polynucleotide constructs, or vectors, each of which encodes one or more prime editing composition components. In some embodiments, the first PEgRNA and/or the second PEgRNA of a prime editing composition is associated with the DNA binding domain, e.g., a Cas9 nickase, of the prime editor. In some embodiments, the first PERNA and/or the second PEgRNA of a prime editing composition complexes with the DNA binding domain of a prime editor and directs the prime editor to the double-stranded target DNA.

In some embodiments, a prime editing composition comprises one or more polynucleotides that encode prime editor components and/or the first PERNA and/or the second PEgRNAs. In some embodiments, a prime editing composition comprises a polynucleotide encoding a fusion protein comprising a DNA binding domain and a DNA polymerase domain. In some embodiments, a prime editing composition comprises (i) a polynucleotide encoding a fusion protein comprising a DNA binding domain and a DNA polymerase domain, (ii) a first PEgRNA or a polynucleotide encoding the first PEgRNA, and (iii) a second PEgRNA or a polynucleotide encoding the second PEgRNA. In some embodiments, a prime editing composition comprises (i) a polynucleotide encoding a fusion protein comprising a DNA binding domain and a DNA polymerase domain, (ii) a first PEgRNA or a polynucleotide encoding the first PEgRNA, and (iii) a second PEgRNA or a polynucleotide encoding the second PEgRNA. In some embodiments, a prime editing composition comprises (i) a polynucleotide encoding a DNA binding domain of a prime editor, e.g., a Cas9 nickase, (ii) a polynucleotide encoding a DNA polymerase domain of a prime editor, e.g., a reverse transcriptase, (iii) a first PEgRNA or a polynucleotide encoding the first PEgRNA, and (iv) a second PEgRNA or a polynucleotide encoding the second PEgRNA. In some embodiments, a prime editing composition comprises (i) a polynucleotide encoding a DNA binding domain of a prime editor, e.g., a Cas9 nickase, (ii) a polynucleotide encoding a DNA polymerase domain of a prime editor, e.g., a reverse transcriptase, (iii) a first PEgRNA or a polynucleotide encoding the first PEgRNA, and (iv) a second PEgRNA or a polynucleotide encoding the second PEgRNA.

In some embodiments, the polynucleotide encoding the DNA binding domain or the polynucleotide encoding the DNA polymerase domain further encodes an additional polypeptide domain, e.g., an RNA-protein recruitment domain, such as a MS2 coat protein domain. In some embodiments, a prime editing composition comprises (i) a polynucleotide encoding a N-terminal half of a prime editor fusion protein and an intein-N and (ii) a polynucleotide encoding a C-terminal half of a prime editor fusion protein and an intein-C. In some embodiments, a prime editing composition comprises (i) a polynucleotide encoding a N-terminal half of a prime editor fusion protein and an intein-N(ii) a polynucleotide encoding a C-terminal half of a prime editor fusion protein and an intein-C, (iii) a first PEgRNA or a polynucleotide encoding the first PEgRNA, and (iv) a second PEgRNA or a polynucleotide encoding the second PEgRNA. In some embodiments, a prime editing composition comprises (i) a polynucleotide encoding a N-terminal portion of a DNA binding domain and an intein-N, (ii) a polynucleotide encoding a C-terminal portion of the DNA binding domain, an intein-C, and a DNA polymerase domain. In some embodiments, the DNA binding domain is a Cas protein domain, e.g., a Cas9 nickase. In some embodiments, the prime editing composition comprises (i) a polynucleotide encoding a N-terminal portion of a DNA binding domain and an intein-N, (ii) a polynucleotide encoding a C-terminal portion of the DNA binding domain, an intein-C, and a DNA polymerase domain, (iii) a first PEgRNA or a polynucleotide encoding the first PEgRNA, and (iv) a second PEgRNA or a polynucleotide encoding the second PERNA.

The editing template of the first PEgRNA, the “first editing template”, and the editing template of the second PEgRNA, the “second editing template”, of a prime editing system may or may not have sequence complementarity to each other. In some embodiments, the first editing template has a region of complementarity or substantial complementarity to the second editing template. In some embodiments, the region of complementarity or substantial complementarity to the second editing template is 2, 3, 4, 5, 6, 7, 8, 9, 10, 11, 12, 13, 14, 15, 16, 17, 18, 19, 20, 21, 22, 23, 24, 25, 30, 35, 40, 45, 50 or more nucleotides in length. In some embodiments, the region of complementarity or substantial complementarity to the second editing template is about 5 to 10, 5 to 15, 5 to 20, 5 to 25, 5 to 30, 5 to 35, 5 to 40, 5 to 45, 5 to 50, 5 to 55, 5 to 60, 5 to 65, 5 to 70, 5 to 75, 5 to 80, 5 to 85, 5 to 90, 5 to 95, 5 to 100, 5 to 110, 5 to 120, 5 to 130, 5 to 140, 5 to 150, 15 to 20, 15 to 25, 15 to 30, 15 to 35, 15 to 40, 15 to 45, 15 to 50, 15 to 55, 15 to 60, 15 to 65, 15 to 70, 15 to 75, 15 to 80, 15 to 85, 15 to 90, 15 to 95, 15 to 100, 15 to 110, 15 to 120, 15 to 130, 15 to 140, 15 to 150, 25 to 30, 25 to 35, 25 to 40, 25 to 45, 25 to 50, 25 to 55, 25 to 60, 25 to 65, 25 to 70, 25 to 75, 25 to 80, 25 to 85, 25 to 90, 25 to 95, 25 to 100, 25 to 110, 25 to 120, 25 to 130, 25 to 140, 25 to 150, 35 to 40, 35 to 45, 35 to 50, 35 to 55, 35 to 60, 35 to 65, 35 to 70, 35 to 75, 35 to 80, 35 to 85, 35 to 90, 35 to 95, 35 to 100, 35 to 110, 35 to 120, 35 to 130, 35 to 140, 35 to 150, 45 to 50, 45 to 55, 45 to 60, 45 to 65, 45 to 70, 45 to 75, 45 to 80, 45 to 85, 45 to 90, 45 to 95, 45 to 100, 45 to 110, 45 to 120, 45 to 130, 45 to 140, o45 to 150, 55 to 60, 55 to 65, 55 to 70, 55 to 75, 55 to 80, 55 to 85, 55 to 90, 55 to 95, 55 to 100, 55 to 110, 55 to 120, 55 to 130, 55 to 140, 55 to 150, 65 to 70, 65 to 75, 65 to 80, 65 to 85, 65 to 90, 65 to 95, 65 to 100, 65 to 110, 65 to 120, 65 to 130, 65 to 140, 65 to 150, 75 to 80, 75 to 85, 75 to 90, 75 to 95, 75 to 100, 75 to 110, 75 to 120, 75 to 130, 75 to 140, 75 to 150, 85 to 90, 85 to 95, 85 to 100, 85 to 110, 85 to 120, 85 to 130, 85 to 140, 85 to 150, 95 to 100, 95 to 110, 95 to 120, 95 to 130, 95 to 140, 95 to 150, 105 to 110, 105 to 120, 105 to 130, 105 to 140, 105 to 150, 115 to 120, 115 to 130, 115 to 140, 115 to 150, 125 to 130, 125 to 140, 125 to 150, 135 to 140, 135 to 150, or 145 to 150 nucleotides in length. In some embodiments, the region of complementarity or substantial complementarity to the second editing template is 10, 11, 12, 13, 14, 15, 16, 17, 18, 19, 20, 21, 22, 23, 24 or 25 nucleotides in length. In some embodiments, the region of complementarity or substantial complementarity to the second editing template is 30, 35, 40, 50, 60, 70, 80, 90, or 100 nucleotides in length. In some embodiments, the region of complementarity or substantial complementarity to the second editing template is at most 20, 25, 30, 35, 40, 50, 60, 70, 80, 90, or 100 nucleotides in length. In some embodiments, the region of complementarity or substantial complementarity to the second editing template is at least 10 nucleotides in length. In some embodiments, the region of complementarity or substantial complementarity to the second editing template is at least 15 nucleotides in length. In some embodiments, the region of complementarity or substantial complementarity to the second editing template is at least 20 nucleotides in length.

In some embodiments, the first editing template has a region of complementarity to the second editing template and does not have a region of complementarity or substantial complementarity to the target double-stranded DNA sequence. In some embodiments, the second editing template has a region of complementarity to the first editing template and does not have a region of complementarity or substantial complementarity to the target double-stranded DNA sequence.

In some embodiments, the first editing template comprises a region of complementarity or substantial complementarity to the target double-stranded DNA sequence. In some embodiments, the first editing template has a region of complementarity or substantial complementarity to the second editing template and has a region of complementarity or substantial complementarity to the target double-stranded DNA sequence.

In some embodiments, the second editing template comprises a region of complementarity or substantial complementarity to the target double-stranded DNA sequence. In some embodiments, the second editing template has a region of complementarity to the first editing template and has a region of complementarity or substantial complementarity to the target double-stranded DNA sequence.

The region of complementarity or substantial complementarity of the first editing template to the double-stranded target DNA sequence may or may not have the same length as the region of complementarity or substantial complementarity of the second editing template to the double-stranded target DNA sequence.

In some embodiments, the region of complementarity or substantial complementarity of the first editing template to the target double-stranded DNA sequence is about 5 to 10, 5 to 15, 5 to 20, 5 to 25, 5 to 30, 5 to 35, 5 to 40, 5 to 45, 5 to 50, 5 to 55, 5 to 60, 5 to 65, 5 to 70, 5 to 75, 5 to 80, 5 to 85, 5 to 90, 5 to 95, 5 to 100, 5 to 110, 5 to 120, 5 to 130, 5 to 140, 5 to 150, 5 to 175, 5 to 200, 5 to 225, 5 to 250, 5 to 275, 5 to 300, 5 to 325, 5 to 350, 5 to 375, 5 to 400, 5 to 425, 5 to 450, 5 to 475, 5 to 500, 10 to 15, 10 to 20, 10 to 25, 10 to 30, 10 to 35, 10 to 40, 10 to 45, 10 to 50, 10 to 55, 10 to 60, 10 to 65, 10 to 70, 10 to 75, 10 to 80, 10 to 85, 10 to 90, 10 to 95, 10 to 100, 10 to 110, 10 to 120, 10 to 130, 10 to 140, 10 to 150, 10 to 175, 10 to 200, 10 to 225, 10 to 250, 10 to 275, 10 to 300, 10 to 325, 10 to 350, 10 to 375, 10 to 400, 10 to 425, 10 to 450, 10 to 475, 10 to 500, 15 to 20, 15 to 25, 15 to 30, 15 to 35, 15 to 40, 15 to 45, 15 to 50, 15 to 55, 15 to 60, 15 to 65, 15 to 70, 15 to 75, 15 to 80, 15 to 85, 15 to 90, 15 to 95, 15 to 100, 15 to 110, 15 to 120, 15 to 130, 15 to 140, 15 to 150, 15 to 175, 15 to 200, 15 to 225, 15 to 250, 15 to 275, 15 to 300, 15 to 325, 15 to 350, 15 to 375, 15 to 400, 15 to 425, 15 to 450, 15 to 475, 15 to 500, 20 to 25, 20 to 30, 20 to 35, 20 to 40, 20 to 45, 20 to 50, 20 to 55, 20 to 60, 20 to 65, 20 to 70, 20 to 75, 20 to 80, 20 to 85, 20 to 90, 20 to 95, 20 to 100, 20 to 110, 20 to 120, 20 to 130, 20 to 140, 20 to 150, 20 to 175, 20 to 200, 20 to 225, 20 to 250, 20 to 275, 20 to 300, 20 to 325, 20 to 350, 20 to 375, 20 to 400, 20 to 425, 20 to 450, 20 to 475, 20 to 500, 30 to 35, 30 to 40, 30 to 45, 30 to 50, 30 to 55, 30 to 60, 30 to 65, 30 to 70, 30 to 75, 30 to 80, 30 to 85, 30 to 90, 30 to 95, 30 to 100, 30 to 110, 30 to 120, 30 to 130, 30 to 140, 30 to 150, 30 to 175, 30 to 200, 30 to 225, 30 to 250, 30 to 275, 30 to 300, 30 to 325, 30 to 350, 30 to 375, 30 to 400, 30 to 425, 30 to 450, 30 to 475, 30 to 500, 40 to 45, 40 to 50, 40 to 55, 40 to 60, 40 to 65, 40 to 70, 40 to 75, 40 to 80, 40 to 85, 40 to 90, 40 to 95, 40 to 100, 40 to 110, 40 to 120, 40 to 130, 40 to 140, 40 to 150, 40 to 175, 40 to 200, 40 to 225, 40 to 250, 40 to 275, 40 to 300, 40 to 325, 40 to 350, 40 to 375, 40 to 400, 40 to 425, 40 to 450, 40 to 475, 40 to 500, 50 to 55, 50 to 60, 50 to 65, 50 to 70, 50 to 75, 50 to 80, 50 to 85, 50 to 90, 50 to 95, 50 to 100, 50 to 110, 50 to 120, 50 to 130, 50 to 140, 50 to 150, 50 to 175, 50 to 200, 50 to 225, 50 to 250, 50 to 275, 50 to 300, 50 to 325, 50 to 350, 50 to 375, 50 to 400, 50 to 425, 50 to 450, 50 to 475, 50 to 500, 75 to 80, 75 to 85, 75 to 90, 75 to 95, 75 to 100, 75 to 110, 75 to 120, 75 to 130, 75 to 140, 75 to 150, 75 to 175, 75 to 200, 75 to 225, 75 to 250, 75 to 275, 75 to 300, 75 to 325, 75 to 350, 75 to 375, 75 to 400, 75 to 425, 75 to 450, 75 to 475, 75 to 500, 100 to 110, 100 to 120, 100 to 130, 100 to 140, 100 to 150, 100 to 175, 100 to 200, 100 to 225, 100 to 250, 100 to 275, 100 to 300, 100 to 325, 100 to 350, 100 to 375, 100 to 400, 100 to 425, 100 to 450, 100 to 475, 100 to 500, 125 to 150, 125 to 175, 125 to 200, 125 to 225, 125 to 250, 125 to 275, 125 to 300, 125 to 325, 125 to 350, 125 to 375, 125 to 400, 125 to 425, 125 to 450, 125 to 475, 125 to 500, 150 to 175, 150 to 200, 150 to 225, 150 to 250, 150 to 275, 150 to 300, 150 to 325, 150 to 350, 150 to 375, 150 to 400, 150 to 425, 150 to 450, 150 to 475, 150 to 500, 175 to 200, 175 to 225, 175 to 250, 175 to 275, 175 to 300, 175 to 325, 175 to 350, 175 to 375, 175 to 400, 175 to 425, 175 to 450, 175 to 475, 175 to 500, 200 to 250, 200 to 275, 200 to 300, 200 to 325, 200 to 350, 200 to 375, 200 to 400, 200 to 425, 200 to 450, 200 to 475, 200 to 500, 225 to 250, 225 to 275, 225 to 300, 225 to 325, 225 to 350, 225 to 375, 225 to 400, 225 to 425, 225 to 450, 225 to 475, 225 to 500, 250 to 275, 250 to 300, 275 to 300, 275 to 325, 275 to 350, 275 to 375, 275 to 400, 275 to 425, 275 to 450, 275 to 475, 275 to 500, 300 to 325, 300 to 350, 300 to 375, 300 to 400, 300 to 425, 300 to 450, 300 to 475, 300 to 500, 325 to 350, 325 to 375, 325 to 400, 325 to 425, 325 to 450, 325 to 475, 325 to 500, 350 to 375, 350 to 400, 350 to 425, 350 to 450, 350 to 475, 350 to 500, 375 to 400, 375 to 425, 375 to 450, 375 to 475, 375 to 500, 400 to 425, 400 to 450, 400 to 475, 400 to 500, 425 to 450, 425 to 475, 425 to 500, 450 to 475, 450 to 500, or 475 to 500 nucleotides in length. In some embodiments, the region of complementarity or substantial complementarity of the first editing template to the target double-stranded DNA sequence is about 10 nucleotides in length. In some embodiments, the region of complementarity or substantial complementarity of the first editing template to the target double-stranded DNA sequence is about 15 nucleotides in length. In some embodiments, the region of complementarity or substantial complementarity of the first editing template to the target double-stranded DNA sequence is about 20 nucleotides in length. In some embodiments, the region of complementarity or substantial complementarity of the first editing template to the target double-stranded DNA sequence is about 21, 22, 23, 24, or 25 nucleotides in length.

In some embodiments, the region of complementarity or substantial complementarity of the second editing template to the target double-stranded DNA sequence is about 5 to 10, 5 to 15, 5 to 20, to 25, 5 to 30, 5 to 35, 5 to 40, 5 to 45, 5 to 50, 5 to 55, 5 to 60, 5 to 65, 5 to 70, 5 to 75, 5 to 80, 5 to 85, 5 to 90, 5 to 95, 5 to 100, 5 to 110, 5 to 120, 5 to 130, 5 to 140, 5 to 150, 5 to 175, 5 to 200, 5 to 225, 5 to 250, 5 to 275, 5 to 300, 5 to 325, 5 to 350, 5 to 375, 5 to 400, 5 to 425, 5 to 450, 5 to 475, 5 to 500, 10 to 15, 10 to 20, 10 to 25, 10 to 30, 10 to 35, 10 to 40, 10 to 45, 10 to 50, 10 to 55, 10 to 60, to 65, 10 to 70, 10 to 75, 10 to 80, 10 to 85, 10 to 90, 10 to 95, 10 to 100, 10 to 110, 10 to 120, 10 to 130, 10 to 140, 10 to 150, 10 to 175, 10 to 200, 10 to 225, 10 to 250, 10 to 275, 10 to 300, 10 to 325, 10 to 350, 10 to 375, 10 to 400, 10 to 425, 10 to 450, 10 to 475, 10 to 500, 15 to 20, 15 to 25, 15 to 30, 15 to 35, 15 to 40, 15 to 45, 15 to 50, 15 to 55, 15 to 60, 15 to 65, 15 to 70, 15 to 75, 15 to 80, to 85, 15 to 90, 15 to 95, 15 to 100, 15 to 110, 15 to 120, 15 to 130, 15 to 140, 15 to 150, 15 to 175, 15 to 200, 15 to 225, 15 to 250, 15 to 275, 15 to 300, 15 to 325, 15 to 350, 15 to 375, 15 to 400, 15 to 425, 15 to 450, 15 to 475, 15 to 500, 20 to 25, 20 to 30, 20 to 35, 20 to 40, 20 to 45, 20 to 50, 20 to 55, 20 to 60, 20 to 65, 20 to 70, 20 to 75, 20 to 80, 20 to 85, 20 to 90, 20 to 95, 20 to 100, 20 to 110, 20 to 120, 20 to 130, 20 to 140, 20 to 150, 20 to 175, 20 to 200, 20 to 225, 20 to 250, 20 to 275, to 300, 20 to 325, 20 to 350, 20 to 375, 20 to 400, 20 to 425, 20 to 450, 20 to 475, 20 to 500, 30 to 35, 30 to 40, 30 to 45, 30 to 50, 30 to 55, 30 to 60, 30 to 65, 30 to 70, 30 to 75, 30 to 80, 30 to 85, 30 to 90, 30 to 95, 30 to 100, 30 to 110, 30 to 120, 30 to 130, 30 to 140, 30 to 150, 30 to 175, 30 to 200, 30 to 225, 30 to 250, 30 to 275, 30 to 300, 30 to 325, 30 to 350, 30 to 375, 30 to 400, 30 to 425, 30 to 450, 30 to 475, 30 to 500, 40 to 45, 40 to 50, 40 to 55, 40 to 60, 40 to 65, 40 to 70, 40 to 75, 40 to 80, 40 to 85, 40 to 90, 40 to 95, 40 to 100, 40 to 110, 40 to 120, 40 to 130, 40 to 140, 40 to 150, 40 to 175, 40 to 200, 40 to 225, 40 to 250, 40 to 275, 40 to 300, 40 to 325, 40 to 350, 40 to 375, 40 to 400, 40 to 425, 40 to 450, 40 to 475, 40 to 500, 50 to 55, 50 to 60, 50 to 65, 50 to 70, 50 to 75, 50 to 80, 50 to 85, 50 to 90, 50 to 95, 50 to 100, 50 to 110, 50 to 120, 50 to 130, 50 to 140, 50 to 150, 50 to 175, 50 to 200, 50 to 225, 50 to 250, 50 to 275, 50 to 300, 50 to 325, 50 to 350, 50 to 375, 50 to 400, 50 to 425, 50 to 450, 50 to 475, 50 to 500, 75 to 80, 75 to 85, 75 to 90, 75 to 95, 75 to 100, 75 to 110, 75 to 120, 75 to 130, 75 to 140, 75 to 150, 75 to 175, 75 to 200, 75 to 225, 75 to 250, 75 to 275, 75 to 300, 75 to 325, 75 to 350, 75 to 375, 75 to 400, 75 to 425, 75 to 450, 75 to 475, 75 to 500, 100 to 110, 100 to 120, 100 to 130, 100 to 140, 100 to 150, 100 to 175, 100 to 200, 100 to 225, 100 to 250, 100 to 275, 100 to 300, 100 to 325, 100 to 350, 100 to 375, 100 to 400, 100 to 425, 100 to 450, 100 to 475, 100 to 500, 125 to 150, 125 to 175, 125 to 200, 125 to 225, 125 to 250, 125 to 275, 125 to 300, 125 to 325, 125 to 350, 125 to 375, 125 to 400, 125 to 425, 125 to 450, 125 to 475, 125 to 500, 150 to 175, 150 to 200, 150 to 225, 150 to 250, 150 to 275, 150 to 300, 150 to 325, 150 to 350, 150 to 375, 150 to 400, 150 to 425, 150 to 450, 150 to 475, 150 to 500, 175 to 200, 175 to 225, 175 to 250, 175 to 275, 175 to 300, 175 to 325, 175 to 350, 175 to 375, 175 to 400, 175 to 425, 175 to 450, 175 to 475, 175 to 500, 200 to 250, 200 to 275, 200 to 300, 200 to 325, 200 to 350, 200 to 375, 200 to 400, 200 to 425, 200 to 450, 200 to 475, 200 to 500, 225 to 250, 225 to 275, 225 to 300, 225 to 325, 225 to 350, 225 to 375, 225 to 400, 225 to 425, 225 to 450, 225 to 475, 225 to 500, 250 to 275, 250 to 300, 275 to 300, 275 to 325, 275 to 350, 275 to 375, 275 to 400, 275 to 425, 275 to 450, 275 to 475, 275 to 500, 300 to 325, 300 to 350, 300 to 375, 300 to 400, 300 to 425, 300 to 450, 300 to 475, 300 to 500, 325 to 350, 325 to 375, 325 to 400, 325 to 425, 325 to 450, 325 to 475, 325 to 500, 350 to 375, 350 to 400, 350 to 425, 350 to 450, 350 to 475, 350 to 500, 375 to 400, 375 to 425, 375 to 450, 375 to 475, 375 to 500, 400 to 425, 400 to 450, 400 to 475, 400 to 500, 425 to 450, 425 to 475, 425 to 500, 450 to 475, 450 to 500, or 475 to 500 nucleotides in length. In some embodiments, the region of complementarity or substantial complementarity of the second editing template to the target double-stranded DNA sequence is about 10 nucleotides in length. In some embodiments, the region of complementarity or substantial complementarity of the second editing template to the target double-stranded DNA sequence is about 15 nucleotides in length. In some embodiments, the region of complementarity or substantial complementarity of the second editing template to the target double-stranded DNA sequence is about 20 nucleotides in length. In some embodiments, the region of complementarity or substantial complementarity of the second editing template to the target double-stranded DNA sequence is about 21, 22, 23, 24, or 25 nucleotides in length.

In some embodiments, the first editing template comprises a region that does not have complementarity or substantial complementarity (the non-complementarity region) to the second editing template. In some embodiments, the first editing template comprises a region of complementarity or substantial complementarity to the second editing template, and further comprises a non-complementarity region to the second editing template. In some embodiments, the first editing template comprises a region of complementarity or substantial complementarity to the second editing template, a non-complementarity region to the second editing template, and a region of complementarity or substantial complementarity to the target double-stranded DNA sequence.

In some embodiments, the second editing template comprises a region that does not have complementarity or substantial complementarity (the non-complementarity region) to the first editing template. In some embodiments, the second editing template comprises a region of complementarity or substantial complementarity to the first editing template, and further comprises a non-complementarity region to the first editing template. In some embodiments, the second editing template comprises a region of complementarity or substantial complementarity to the first editing template, a non-complementarity region to the first editing template, and a region of complementarity or substantial complementarity to the target double-stranded DNA sequence.

In some embodiments, the region of non-complementarity of the first editing template to the second editing template and the region of non-complementarity of the second editing template to the first editing template are of the same length. In some embodiments, the region of non-complementarity of the first editing template to the second editing template and the region of non-complementarity of the second editing template to the first editing template are of different lengths. In some embodiments, the first editing template and the second editing template both comprise a region of non-complementarity to each other. In some embodiments, the first editing template comprises a region of non-complementarity to the second editing template, and the second editing template does not comprise a region of non-complementarity to the first editing template. In some embodiments, the second editing template comprises a region of non-complementarity to the first editing template, and the first editing template does not comprise a region of non-complementarity to the second editing template. For example, the first editing template may be complementary or substantially complementary to the second editing template through its entire length, while the second editing template comprises a region that does not have complementarity to the first editing template, or vice versa.

In some embodiments, the region of non-complementarity of the first editing template to the second editing template is about 5 to 10, 5 to 15, 5 to 20, 5 to 25, 5 to 30, 5 to 35, 5 to 40, 5 to 45, 5 to 50, 5 to 55, 5 to 60, 5 to 65, 5 to 70, 5 to 75, 5 to 80, 5 to 85, 5 to 90, 5 to 95, 5 to 100, 5 to 110, 5 to 120, 5 to 130, 5 to 140, 5 to 150, 5 to 175, 5 to 200, 5 to 225, 5 to 250, 5 to 275, 5 to 300, 5 to 325, 5 to 350, 5 to 375, 5 to 400, 5 to 425, 5 to 450, 5 to 475, 5 to 500, 10 to 15, 10 to 20, 10 to 25, 10 to 30, 10 to 35, 10 to 40, 10 to 45, 10 to 50, 10 to 55, 10 to 60, 10 to 65, 10 to 70, 10 to 75, 10 to 80, 10 to 85, 10 to 90, 10 to 95, 10 to 100, 10 to 110, 10 to 120, 10 to 130, 10 to 140, 10 to 150, 10 to 175, 10 to 200, 10 to 225, 10 to 250, 10 to 275, 10 to 300, 10 to 325, 10 to 350, 10 to 375, 10 to 400, 10 to 425, 10 to 450, 10 to 475, 10 to 500, 15 to 20, 15 to 25, 15 to 30, 15 to 35, 15 to 40, 15 to 45, 15 to 50, 15 to 55, 15 to 60, 15 to 65, 15 to 70, 15 to 75, 15 to 80, 15 to 85, 15 to 90, 15 to 95, 15 to 100, 15 to 110, 15 to 120, 15 to 130, 15 to 140, 15 to 150, 15 to 175, 15 to 200, 15 to 225, 15 to 250, 15 to 275, 15 to 300, 15 to 325, 15 to 350, 15 to 375, 15 to 400, 15 to 425, 15 to 450, 15 to 475, 15 to 500, 20 to 25, 20 to 30, 20 to 35, 20 to 40, 20 to 45, 20 to 50, 20 to 55, 20 to 60, 20 to 65, 20 to 70, 20 to 75, 20 to 80, 20 to 85, 20 to 90, 20 to 95, 20 to 100, 20 to 110, 20 to 120, 20 to 130, 20 to 140, 20 to 150, 20 to 175, 20 to 200, 20 to 225, 20 to 250, 20 to 275, 20 to 300, 20 to 325, 20 to 350, 20 to 375, 20 to 400, 20 to 425, 20 to 450, 20 to 475, 20 to 500, 30 to 35, 30 to 40, 30 to 45, 30 to 50, 30 to 55, 30 to 60, 30 to 65, 30 to 70, 30 to 75, 30 to 80, 30 to 85, 30 to 90, 30 to 95, 30 to 100, 30 to 110, 30 to 120, 30 to 130, 30 to 140, 30 to 150, 30 to 175, 30 to 200, 30 to 225, 30 to 250, 30 to 275, 30 to 300, 30 to 325, 30 to 350, 30 to 375, 30 to 400, 30 to 425, 30 to 450, 30 to 475, 30 to 500, 40 to 45, 40 to 50, 40 to 55, 40 to 60, 40 to 65, 40 to 70, 40 to 75, 40 to 80, 40 to 85, 40 to 90, 40 to 95, 40 to 100, 40 to 110, 40 to 120, 40 to 130, 40 to 140, 40 to 150, 40 to 175, 40 to 200, 40 to 225, 40 to 250, 40 to 275, 40 to 300, 40 to 325, 40 to 350, 40 to 375, 40 to 400, 40 to 425, 40 to 450, 40 to 475, 40 to 500, 50 to 55, 50 to 60, 50 to 65, 50 to 70, 50 to 75, 50 to 80, 50 to 85, 50 to 90, 50 to 95, 50 to 100, 50 to 110, 50 to 120, 50 to 130, 50 to 140, 50 to 150, 50 to 175, 50 to 200, 50 to 225, 50 to 250, 50 to 275, 50 to 300, 50 to 325, 50 to 350, 50 to 375, 50 to 400, 50 to 425, 50 to 450, 50 to 475, 50 to 500, 75 to 80, 75 to 85, 75 to 90, 75 to 95, 75 to 100, 75 to 110, 75 to 120, 75 to 130, 75 to 140, 75 to 150, 75 to 175, 75 to 200, 75 to 225, 75 to 250, 75 to 275, 75 to 300, 75 to 325, 75 to 350, 75 to 375, 75 to 400, 75 to 425, 75 to 450, 75 to 475, 75 to 500, 100 to 110, 100 to 120, 100 to 130, 100 to 140, 100 to 150, 100 to 175, 100 to 200, 100 to 225, 100 to 250, 100 to 275, 100 to 300, 100 to 325, 100 to 350, 100 to 375, 100 to 400, 100 to 425, 100 to 450, 100 to 475, 100 to 500, 125 to 150, 125 to 175, 125 to 200, 125 to 225, 125 to 250, 125 to 275, 125 to 300, 125 to 325, 125 to 350, 125 to 375, 125 to 400, 125 to 425, 125 to 450, 125 to 475, 125 to 500, 150 to 175, 150 to 200, 150 to 225, 150 to 250, 150 to 275, 150 to 300, 150 to 325, 150 to 350, 150 to 375, 150 to 400, 150 to 425, 150 to 450, 150 to 475, 150 to 500, 175 to 200, 175 to 225, 175 to 250, 175 to 275, 175 to 300, 175 to 325, 175 to 350, 175 to 375, 175 to 400, 175 to 425, 175 to 450, 175 to 475, 175 to 500, 200 to 250, 200 to 275, 200 to 300, 200 to 325, 200 to 350, 200 to 375, 200 to 400, 200 to 425, 200 to 450, 200 to 475, 200 to 500, 225 to 250, 225 to 275, 225 to 300, 225 to 325, 225 to 350, 225 to 375, 225 to 400, 225 to 425, 225 to 450, 225 to 475, 225 to 500, 250 to 275, 250 to 300, 275 to 300, 275 to 325, 275 to 350, 275 to 375, 275 to 400, 275 to 425, 275 to 450, 275 to 475, 275 to 500, 300 to 325, 300 to 350, 300 to 375, 300 to 400, 300 to 425, 300 to 450, 300 to 475, 300 to 500, 325 to 350, 325 to 375, 325 to 400, 325 to 425, 325 to 450, 325 to 475, 325 to 500, 350 to 375, 350 to 400, 350 to 425, 350 to 450, 350 to 475, 350 to 500, 375 to 400, 375 to 425, 375 to 450, 375 to 475, 375 to 500, 400 to 425, 400 to 450, 400 to 475, 400 to 500, 425 to 450, 425 to 475, 425 to 500, 450 to 475, 450 to 500, or 475 to 500 nucleotides in length. In some embodiments, the region of non-complementarity of the first editing template to the second editing template is about 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 60, 70, 80, 90, 100, 110, 120, 130, 140, 150, 160, 170, 180, 190, 200, 210, 220,230, 240, 250, 260, 270, 280, 290, 300, 310, 320, 330, 340, 350, 360, 370, 380, 390, 400, 410, 420, 430, 440, 450, 460, 470, 480, 490, or 500 or more nucleotides in length.

In some embodiments, the region of non-complementarity of the second editing template to the first editing template is about 5 to 10, 5 to 15, 5 to 20, 5 to 25, 5 to 30, 5 to 35, 5 to 40, 5 to 45, 5 to 50, 5 to 55, 5 to 60, 5 to 65, 5 to 70, 5 to 75, 5 to 80, 5 to 85, 5 to 90, 5 to 95, 5 to 100, 5 to 110, 5 to 120, 5 to 130, 5 to 140, 5 to 150, 5 to 175, 5 to 200, 5 to 225, 5 to 250, 5 to 275, 5 to 300, 5 to 325, 5 to 350, 5 to 375, 5 to 400, 5 to 425, 5 to 450, 5 to 475, 5 to 500, 10 to 15, 10 to 20, 10 to 25, 10 to 30, 10 to 35, 10 to 40, 10 to 45, 10 to 50, 10 to 55, 10 to 60, 10 to 65, 10 to 70, 10 to 75, 10 to 80, 10 to 85, 10 to 90, 10 to 95, 10 to 100, 10 to 110, 10 to 120, 10 to 130, 10 to 140, 10 to 150, 10 to 175, 10 to 200, 10 to 225, 10 to 250, 10 to 275, 10 to 300, 10 to 325, 10 to 350, 10 to 375, 10 to 400, 10 to 425, 10 to 450, 10 to 475, 10 to 500, 15 to 20, 15 to 25, 15 to 30, 15 to 35, 15 to 40, 15 to 45, 15 to 50, 15 to 55, 15 to 60, 15 to 65, 15 to 70, 15 to 75, 15 to 80, 15 to 85, 15 to 90, 15 to 95, 15 to 100, 15 to 110, 15 to 120, 15 to 130, 15 to 140, 15 to 150, 15 to 175, 15 to 200, 15 to 225, 15 to 250, 15 to 275, 15 to 300, 15 to 325, 15 to 350, 15 to 375, 15 to 400, 15 to 425, 15 to 450, 15 to 475, 15 to 500, 20 to 25, 20 to 30, 20 to 35, 20 to 40, 20 to 45, 20 to 50, 20 to 55, 20 to 60, 20 to 65, 20 to 70, 20 to 75, 20 to 80, 20 to 85, 20 to 90, 20 to 95, 20 to 100, 20 to 110, 20 to 120, 20 to 130, 20 to 140, 20 to 150, 20 to 175, 20 to 200, 20 to 225, 20 to 250, 20 to 275, 20 to 300, 20 to 325, 20 to 350, 20 to 375, 20 to 400, 20 to 425, 20 to 450, 20 to 475, 20 to 500, 30 to 35, 30 to 40, 30 to 45, 30 to 50, 30 to 55, 30 to 60, 30 to 65, 30 to 70, 30 to 75, 30 to 80, 30 to 85, 30 to 90, 30 to 95, 30 to 100, 30 to 110, 30 to 120, 30 to 130, 30 to 140, 30 to 150, 30 to 175, 30 to 200, 30 to 225, 30 to 250, 30 to 275, 30 to 300, 30 to 325, 30 to 350, 30 to 375, 30 to 400, 30 to 425, 30 to 450, 30 to 475, 30 to 500, 40 to 45, 40 to 50, 40 to 55, 40 to 60, 40 to 65, 40 to 70, 40 to 75, 40 to 80, 40 to 85, 40 to 90, 40 to 95, 40 to 100, 40 to 110, 40 to 120, 40 to 130, 40 to 140, 40 to 150, 40 to 175, 40 to 200, 40 to 225, 40 to 250, 40 to 275, 40 to 300, 40 to 325, 40 to 350, 40 to 375, 40 to 400, 40 to 425, 40 to 450, 40 to 475, 40 to 500, 50 to 55, 50 to 60, 50 to 65, 50 to 70, 50 to 75, 50 to 80, 50 to 85, 50 to 90, 50 to 95, 50 to 100, 50 to 110, 50 to 120, 50 to 130, 50 to 140, 50 to 150, 50 to 175, 50 to 200, 50 to 225, 50 to 250, 50 to 275, 50 to 300, 50 to 325, 50 to 350, 50 to 375, 50 to 400, 50 to 425, 50 to 450, 50 to 475, 50 to 500, 75 to 80, 75 to 85, 75 to 90, 75 to 95, 75 to 100, 75 to 110, 75 to 120, 75 to 130, 75 to 140, 75 to 150, 75 to 175, 75 to 200, 75 to 225, 75 to 250, 75 to 275, 75 to 300, 75 to 325, 75 to 350, 75 to 375, 75 to 400, 75 to 425, 75 to 450, 75 to 475, 75 to 500, 100 to 110, 100 to 120, 100 to 130, 100 to 140, 100 to 150, 100 to 175, 100 to 200, 100 to 225, 100 to 250, 100 to 275, 100 to 300, 100 to 325, 100 to 350, 100 to 375, 100 to 400, 100 to 425, 100 to 450, 100 to 475, 100 to 500, 125 to 150, 125 to 175, 125 to 200, 125 to 225, 125 to 250, 125 to 275, 125 to 300, 125 to 325, 125 to 350, 125 to 375, 125 to 400, 125 to 425, 125 to 450, 125 to 475, 125 to 500, 150 to 175, 150 to 200, 150 to 225, 150 to 250, 150 to 275, 150 to 300, 150 to 325, 150 to 350, 150 to 375, 150 to 400, 150 to 425, 150 to 450, 150 to 475, 150 to 500, 175 to 200, 175 to 225, 175 to 250, 175 to 275, 175 to 300, 175 to 325, 175 to 350, 175 to 375, 175 to 400, 175 to 425, 175 to 450, 175 to 475, 175 to 500, 200 to 250, 200 to 275, 200 to 300, 200 to 325, 200 to 350, 200 to 375, 200 to 400, 200 to 425, 200 to 450, 200 to 475, 200 to 500, 225 to 250, 225 to 275, 225 to 300, 225 to 325, 225 to 350, 225 to 375, 225 to 400, 225 to 425, 225 to 450, 225 to 475, 225 to 500, 250 to 275, 250 to 300, 275 to 300, 275 to 325, 275 to 350, 275 to 375, 275 to 400, 275 to 425, 275 to 450, 275 to 475, 275 to 500, 300 to 325, 300 to 350, 300 to 375, 300 to 400, 300 to 425, 300 to 450, 300 to 475, 300 to 500, 325 to 350, 325 to 375, 325 to 400, 325 to 425, 325 to 450, 325 to 475, 325 to 500, 350 to 375, 350 to 400, 350 to 425, 350 to 450, 350 to 475, 350 to 500, 375 to 400, 375 to 425, 375 to 450, 375 to 475, 375 to 500, 400 to 425, 400 to 450, 400 to 475, 400 to 500, 425 to 450, 425 to 475, 425 to 500, 450 to 475, 450 to 500, or 475 to 500 nucleotides in length. In some embodiments, the region of non-complementarity of the second editing template to the first editing template is about 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 60, 70, 80, 90, 100, 110, 120, 130, 140, 150, 160, 170, 180, 190, 200, 210, 220,230, 240, 250, 260, 270, 280, 290, 300, 310, 320, 330, 340, 350, 360, 370, 380, 390, 400, 410, 420, 430, 440, 450, 460, 470, 480, 490, or 500 or more nucleotides in length.

In some embodiments, the first PEgRNA and the second PEgRNA have the same gRNA cores. In some embodiments, the first PERNA and the second PEgRNA have different gRNA cores.

In some embodiments, a prime editing system comprises one or more polynucleotides encoding one or more prime editor polypeptides, wherein activity of the prime editing system may be temporally regulated by controlling the timing in which the vectors are delivered. For example, in some embodiments, a polynucleotide encoding the prime editor and a polynucleotide encoding a PEgRNA or both PEgRNAs may be delivered simultaneously. For example, in some embodiments, a polynucleotide encoding the prime editor and a polynucleotide encoding a PEgRNA or both PEgRNAs may be delivered sequentially.

In some embodiments, a polynucleotide encoding a component of a prime editing system may further comprise an element that is capable of modifying the intracellular half-life of the polynucleotide and/or modulating translational control. In some embodiments, the polynucleotide is a RNA, for example, an mRNA. In some embodiments, the half-life of the polynucleotide, e.g., the RNA may be increased. In some embodiments, the half-life of the polynucleotide, e.g., the RNA may be decreased. In some embodiments, the element may be capable of increasing the stability of the polynucleotide, e.g., the RNA. In some embodiments, the element may be capable of decreasing the stability of the polynucleotide, e.g., the RNA. In some embodiments, the element may be within the 3′ UTR of the RNA. In some embodiments, the element may include a polyadenylation signal (PA). In some embodiments, the element may include a cap, e.g., an upstream mRNA or PEgRNA end. In some embodiments, the RNA may comprise no PA such that it is subject to quicker degradation in the cell after transcription.

In some embodiments, the element may include at least one AU-rich element (ARE). The AREs may be bound by ARE binding proteins in a manner that is dependent upon tissue type, cell type, timing, cellular localization, and environment. In some embodiments the destabilizing element may promote RNA decay, affect RNA stability, or activate translation. In some embodiments, the ARE may comprise 50 to 150 nucleotides in length. In some embodiments, the ARE may comprise at least one copy of the sequence AUUUA. In some embodiments, at least one ARE may be added to the 3′ UTR of the RNA. In some embodiments, the element may be a Woodchuck Hepatitis Virus Posttranscriptional Regulatory Element (WPRE). In further embodiments, the element is a modified and/or truncated WPRE sequence that is capable of enhancing expression from the transcript. In some embodiments, the WPRE or equivalent may be added to the 3′ UTR of the RNA. In some embodiments, the element may be selected from other RNA sequence motifs that are enriched in either fast- or slow-decaying transcripts. In some embodiments, the polynucleotide, e.g., a vector, encoding the PE or the PEgRNA may be self-destroyed via cleavage of a target sequence present on the polynucleotide, e.g., a vector. The cleavage may prevent continued transcription of a PE or a PERNA.

Polynucleotides encoding prime editing composition components can be DNA, RNA, or any combination thereof. In some embodiments, a polynucleotide encoding a prime editing composition component is an expression construct. In some embodiments, a polynucleotide encoding a prime editing composition component is a vector. In some embodiments, the vector is a DNA vector. In some embodiments, the vector is a plasmid. In some embodiments, the vector is a virus vector, e.g., a retroviral vector, adenoviral vector, lentiviral vector, herpesvirus vector, or an adeno-associated virus vector (AAV).

In some embodiments, polynucleotides encoding polypeptide components of a prime editing composition are codon optimized by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. In some embodiments, a polynucleotide encoding a polypeptide component of a prime editing composition are operably linked to one or more expression regulatory elements, for example, a promoter, a 3′ UTR, a 5′ UTR, or any combination thereof. In some embodiments, a polynucleotide encoding a prime editing composition component is a messenger RNA (mRNA). In some embodiments, the mRNA comprises a Cap at the 5′ end and/or a poly A tail at the 3′ end.

In some embodiments, a prime editing system comprises a first PEgRNA, a second PEgRNA, and a nuclease that recognizes the PAM sequence “NG.” In some embodiments, a PAM motif on the edit strand comprises an “NG” motif, wherein N is any nucleotide.

In some embodiments, a prime editing system comprises a first PEgRNA, a second PEgRNA, and a nuclease that recognizes the PAM sequence “NAG.” In some embodiments, a PAM motif on the edit strand comprises an “NAG” motif, wherein N is any nucleotide.

In some embodiments, a prime editing system comprises a first PEgRNA, a second PEgRNA, and a nuclease that recognizes the PAM sequence “NGA.” In some embodiments, a PAM motif on the edit strand comprises an “NGA” motif, wherein N is any nucleotide.

In some embodiments, a prime editing system comprises a first PERNA, a second PEgRNA, and a nuclease that recognizes the PAM sequence “NNGG.” In some embodiments, a PAM motif on the edit strand comprises an “NNGG” motif, wherein N is any nucleotide.

In some embodiments, a prime editing system comprises a first PEgRNA, a second PEgRNA, and a nuclease that recognizes the PAM sequence “NNGRRT.” In some embodiments, a PAM motif on the edit strand comprises an “NNGRRT” motif, wherein N is any nucleotide and R is A or G.

Provided herein in some embodiments are example sequences for PEgRNA spacers, PBS, and editing templates for a prime editing system comprising a nuclease that recognizes the PAM sequence “NGG.” In some embodiments, a PAM motif on the edit strand comprises an “NGG” motif, wherein N is any nucleotide.

### Prime Editing Composition Comprising Recombinases

In some embodiments, the prime editing composition provided herein comprises one or more recombinases, or one or more polynucleotides encoding the recombinases, wherein the PEgRNA or PEgRNA pair of the prime editing composition encode insertion of one or more recombinase recognition sequences (RSSs) in the target gene, e.g., the TRAC gene.

In some embodiments, the recombinase is a tyrosine recombinase. In some embodiments, the recombinase is a serine recombinase. Examples of tyrosine recombinases include, without limitation, Cre, FLP, R, Lambda, HK101, HK022, and pSAM2. Examples of serine recombinases include, without limitation, Si74, No67, Kp03, Pa01, Nm60, BceINTa, BcytINTd, SscINTd, SacINTd, Hin, Gin, Tn3, 13-six, CinH, ParA, y6, Bxbl, OC31, TP901, TG1, pBT1, R4, pRV1, pFC1, MR11, A118, U153, and gp29. Exemplary serine recombinase sequences and corresponding recombinases are provided in Table 5. Amino acid sequences of the recombinases are in Table 27 below (“*” indicates a stop codon):

TABLE 27
Exemplary serine recombinase sequences

| Recombinase | Amino acid sequence |
|---|---|
| Bxb1 | MRALVVIRLSRVTDATTSPERQLESCQQLCAQRGWDVVGVAEDLDVSGAVDPFD |
| | RKRRPNLARWLAFEEQPFDVIVAYRVDRLTRSIRHLQQLVHWAEDHKKLVVSAT |
| | EAHFDTTTPFAAVVIALMGTVAQMELEAIKERNRSAAHFNIRAGKYRGSLPPWGY |
| | LPTRVDGEWRLVPDPVQRERILEVYHRVVDNHEPLHLVAHDLNRRGVLSPKDYF |
| | AQLQGREPQGREWSATALKRSMISEAMLGYATLNGKTVRDDDGAPLVRAEPILT |
| | REQLEALRAELVKTSRAKPAVSTPSLLLRVLFCAVCGEPAYKFAGGGRKHPRYRC |
| | RSMGFPKHCGNGTVAMAEWDAFCEEQVLDLLGDAERLEKVWVAGSDSAVELAE |
| | VNAELVDLTSLIGSPAYRAGSPQREALDARIAALAARQEELEGLEARPSGWEWRE |
| | TGQRFGDWWREQDTAAKNTWLRSMNVRLTFDVRGGLTRTIDFGDLQEYEQHLR |
| | LGSVVERLHTGMS (SEQ ID NO: 1131) |
| Si74 | MQPNLRYLACLRLSADSDGSTSIEWQRGVIRHHVSSPHLSGVLVGEAEDTDVSGS |
| | LSPFKRPKLGKWLTAKADEFDVIIAAKMDRLTRRSMHFNELLEWAQQNGKFIVC |
| | VEEGFDLSTPQGKMMARMTAVFAEAEWDTIQARILNGVQTRLENRSWLVGAPPT |
| | GYRIKTVEGGKRKILEIDQDFYPYVEEIFRRIREGQSTHRIARDENGRSVLTWGDHL |
| | RKLKGEEPKGTQWQATIINKFIRSSWVPGLYTYKGEAVLDDQGDPVILPETPLAT |
| | MDEWTDLVDRIKPAPKPEGATGGSRNSAKSLLSGVAHCGECGAPFTSLMDSGYK |
| | RKDGTKVPGHRRYRCSNKFKGGDCKNGSYVRADVLDSWVDQAIRDSIGQEDMY |
| | ERAGKGPSQARELQETKARLAKLEADYESGKYDGEGQDESYWRMNKNLSAKVA |
| | HLAKQEAERANPTFKATGKKYGEVWEAKDQEDRRDFLRTYGVKVFVWGEGAD |
| | KKDRGYAMNLGDIKTMAEELFPNRDRARFKLVHTHNAPEGYLSKIGIAVGLLKY |
| | GHPLEVKLRSPENS* (SEQ ID NO: 1212) |
| No67 | MPESPPRALIVIRLSKVTDATTSPERQLAECRAICEKRGYEVVGVAEDLDVSGAVD |
| | PFDRKKRPHLARWLHGEHLDDNGEPVPFEVIVVYRVDRLTRSVRHLQKLVAWAD |
| | DHKKLVVSATEAHFDTSLPFSAVLIALIGTVAEMELAGISERNASAARHNIQAGRY |
| | RGSTPPWGYVPSNDTGEWRLVRDKEQADVINEVARRVIEGEPLQKIAHDLTRRGI |
| | LTPKDNFAKQRGREIKGREWSVTQLKRSLLSEAMLGHAVSGGAAVRNDDGSPVV |
| | RSEPILSREIYDRVAAELSSRAKRGEPNKRTSSLLLGVISCGNPCLHKQQGHECPEG |
| | CSGTCDEPVYKFNGGSHSQFPRYRCRTMTRAYKCGNRTIRADQADAQVERTILAL |
| | LGSSERLERVWDAGEDHSAELADINDELVDLTSQLGVGAFRAGTPQRAKLDARIA |
| | SLAERQAQLSSEAVVPAGWKLLPTGELFGDWWSRQDLTARNVWLRSMGVRARF |
| | KRDDKTLYIDLGNLNDLISGLKPGGTAQRVRGGLQAMERNGIQGMVFSDADSEV |
| | MPAPAAGYMWIQPVEGVWVYTSEALLAAAAERQALRREKIEDEFAYGPGDFDED |
| | WD* (SEQ ID NO: 1213) |
| Kp03 | MRQVITYLRFSSKPQERGDSIRRQKGLFERWLKDNPDAKVVDEFSDEGASAYHGH |
| | HLKGDFGRMLQNIQDGKYLSGQTVLLVESETRLNRQKARNTENLVDLITGKGVD |
| | VICLESGKIYTSTNIDDLDTSIQLKIAAHIAHQQSKEKSIKVSAAWEHRAQLALEGK |
| | QQLTKNVPGWIDPDTRKLNEHSSTVVTIFDLLLSGESLHNIARYLQANNIKSFSRRE |
| | KANGFSVHSVRTILRSESTVGTLAASKRNDRPAIPNYYEPAIDVATFNKAQEILSKN |
| | RVGRAPASDNPITINLFKGIIRCQCGASVHPTGVKATYQGVYRCNNVPDGRCNVP |
| | TIKRKPFDKWMLDNIVGFLERDDGNNTDKRKAEIEYQISLVTSKLKKATTLLLELD |
| | DVTELKEQVKELNIQRSNLQSELDELNQRETLSDKPLHHLSEIDLTTKAGRVEAQL |
| | ILSKFVQSIELQREMIIITLRNGTVIGKSRDLSPVLSQDLMKQVVSSPSPTDIDMFSVI |
| | TSDEEFRKSGKQVTKRS* (SEQ ID NO: 1214) |
| Pa01 | MPSAFSYVRFSSGKQAKGSSEHRQRAMLGQWLEQHPSFTLSDLRFEDLGRSGFSG |
| | EHLDHGLGQLLAAIDSGAIKSGDVILVEAVDRIGRLEPLEMLPLFSRIVKAGVSVIT |
| | LEDGHVYDRSSVNETSLFLLVAKIQQAHEYSNRLSRRINASYTARREKAKAGLGIK |
| | RETPVWLTTDGQLVPHVAPHIAQAFQDYADGLGERRICRKLRESGLEEFSKTNAT |
| | TVRRWLKNRTAIGYWNDIPDVYPHVVDPALFYQVQQRLDAPKVDRAKPSAHYLT |
| | GLVKCAVCGRNYNYKQRKHTDPAMLCTSRARLAGEGCSNSKTYPVIVLDQVRKL |
| | TSLPFLQHAMESASSQADPSSQRLAVIDGEIGELSRKISEATKALLVLGFTPEIQESL |
| | EQLKTAREALEEERATLLLPQAEKLTTAQLEAFSNGLLDDEPMKLNHVLQTAGYS |
| | MVVHPDGSIDVDGKRFVYEGASRKEKVYKLRLIGEDKQWSLPILTPQMATYKSLF |
| | MAAVRLPGDPSEEELRRFEEAKHSER* (SEQ ID NO: 1215) |
| Nm60 | MSRPTGLTIDIYLRKSRKDLEEEKKASESGETYDTLERHRRTLFAVAKKERHNIAN |
| | IYEEVVSGESVSERPQIQAMLRNLETSHIEGVLVMDLDRLGRGDMLDQGLLDRAF |
| | RYSGAKILTPTEVYDPESETWELVEGIKSLVAREELKAITRRMQRGRVASAGEGKS |
| | ISKVPPYGYLRDENLRLYPDPETAWVVKKMFEMMRDGHGRIAVAQELDKLGIKP |
| | PNDKRRSWAPSSITAIIKNEVYLGTIIWGQVKYSKRNGKYKKTKLPRSKWTIKENA |
| | HEPIVSRELFEAANRAHTGRWRPSTNATKTLSNPLAGVLKCDVCGFTMLYQPRPN |
| | RPNDFIRCTQPTCQAVQKGATLALVEQHILDGLKQFAQELELQTEVPELDNDKDIA |
| | VKKSLVGNKQEEIAQLETQKSKLHDLLERGIYDVDTFLERQQNLNNRINGLQDDI |
| | RNIESEIKKEEVRNSSVLNLLPQLQTVISEYENADTESKNRLLKSVLEKATYLRKKE |
| | WTKRDQFIIQLYPKI* (SEQ ID NO: 1216) |
| BceINTa | MYPYDVPDYAGSYRPESLDVCIYLRKSRKDVEEERRAIEEGSSYNALERHRKRLF |
| | AIAKAENHNIIDIFEEVASGESIQERPQMQQLLRKLEGNEIDGVLVIDLDRLGRGDM |
| | LDAGMIDRAFRYSSTKIITPTDVYDPDDESWELVFGIKSLISRQELKSITKRLQNGRI |
| | DSVKEGKHIGKKPPYGYLKDENLRLYPDPEKAWIVKKIFELMCDGKGRQMIAAEL |
| | DRLGIDPPVTKRGAWDSSTITSIIKNEVYTGVIVWGKFKHKKRNGKYTRHKNPQE |
| | KWIMYENAHEPIISKELFDAANEAHSSRHKPAVITSKKLTNPLAGILKCKLCGYTM |
| | LIQTRKDRPHNYLRCNNPACKGKQKQSVFNLVEEKLLYSLQQIVDEYQAQKVEE |
| | VEIDDSKLISFKEKAIISKEKELKELQAQKGNLHDLLEQGIYTVEIFLERQKNLVERI |
| | TSIENDIEVLQKEIETEQIKEHNKTEFIPALKTVIESYHKTTNIELKNQLLKTILSTVT |
| | YYRHPDWKTNEFEIQVYFKIS* (SEQ ID NO: 1217) |
| BcytINTd | MYPYDVPDYAGSAVGIYIRVSTQEQASEGHSIESQKKKLASYCEIQGWDDYRFYIE |
| | EGISGKNTNRPKLKLLMEHIEKGKINILLVYRLDRLTRSVIDLHKLLNFLQEHGCAF |
| | KSATETYDTTTANGRMSMGIVSLLAQWETENMSERIKLNLEHKVLVEGERVGAIP |
| | YGFDLSDDEKLVKNEKSAILLDMVERVENGWSVNRIVNYLNLTNNDRNWSPNGV |
| | LRLLRNPALYGATRWNDKIAENTHEGIISKERFNRLQQILADRSIHHRRDVKGTYIF |
| | QGVLRCPVCDQTLSVNRFIKKRKDGTEYCGVLYRCQPCIKQNKYNLAIGEARFLK |
| | ALNEYMSTVEFQTVEDEVIPKKSEREMLESQLQQIARKREKYQKAWASDLMSDD |
| | EFEKLMVETRETYDECKQKLESCEDPIKIDETYLKEIVYMFHQTFNDLESEKQKEFI |
| | SKFIRTIRYTVKEQQPIRPDKSKTGKGKQKVIITEVEFYQS* (SEQ ID NO: 1218) |
| SscINTd | MNEKNLEIGAAYIRVSTDDQTELSPDAQLRVILEAAKKDGIIIPQEFVFMEDRGRSG |
| | RRADNRPEFQRMISTARQNPSPFRYLYLWKFSRFARNQEESAFYKGILRKKCGVTI |
| | KSVSEPIMEGMFGRLVEMIIEWSDEFYSVNLSGEVLRGMTQKALEHGYQLTPCLG |
| | YDAVGHGRPYVINEEQYQIVEFIHRSFFDGKDMTWIAREANRRGYHTRRGNPFDT |
| | RAVRIILTNSFYVGLVKWNDVTFQGTHECRESVTSVFSANQERLNRIHRPRGRRQ |
| | ASSCKHWLSGLLKCSICGASLGYNQTKDLTKRGHAFQCWKYTKGIHPGSCSVSSL |
| | KAEAAVLESLQMILETGEVEYTYEQREKHLDDNKLTLIQKSLERLDTKELRIREAY |
| | ESGIDTLDEFKTNKARLQRERDQLMEELEELHSQEEPEDVPGKEILIERIQNVYDLL |
| | QSPDVDNDDKGNAVRSIIKKIVYIKESKTFCFYYYV* (SEQ ID NO: 1219) |
| SacINTd | MKEKVSERKTGAIYIRVSTDKQEELSPDAQLRLLLDYAKKDSIDVPKEYIFQDNGI |
| | SGRKANKRPAFQNMIALAKSKEHPIDTIIVWKFSRFARNQEESIVYKSLLKKNNVD |
| | VVSVSEPLIDGPFGSLIERIIEWMDEYYSIRLSGEVMRGMTQNAMRGHYQSDAPIG |
| | YTSPGDKKPPVINPDTVQIPLMIKDMFLSGSTQLQIARKLNDSGYRTKRGNLWDA |
| | RGVRYVLENPFYIGKSRWNYTERGRRLKPADEVIYADGNWEALWDEDTFKEIQK |
| | RLALNMRKSKSRDISAAKHWLSGLLICSSCGGTLAFGGAHNMRGFQCWKYSKGF |
| | CSESHYISTGPIEKMVLEYLEAVMHSPALSYTVISSSSVDASSKLSDLERQLQKIDA |
| | KEKRIKAAYLNEIDTLEEYKANKTALEEERRTVEKEIEELTLSDVKYSKEDLDKK |
| | MKQNISDLLRVLRDESADYIQKGNMMRNVVDHIVFNRKNTSLDVFLKLVV* (SEQ |
| | ID NO: 1220) |

In some embodiments, the prime editing composition further comprises a DNA polynucleotide for recombination with the target gene containing the RSS(s) introduced by prime editing. In some embodiments, the DNA polynucleotide comprises (a) a donor sequence and (b) one or more RSSs recognized by the recombinase. In some embodiments, the prime editing composition comprises a recombinase that recognizes a first RSS introduced into the TRAC gene by prime editing, and further comprises a DNA polynucleotide comprising (a) a donor sequence and (b) a second RSS recognized by the same recombinase. In some embodiments, the first RSS is an attB sequence, and the second RSS is an attP sequence.

In some embodiments, the recombinase is Bxb1. In some embodiments, the RSS is an attB sequence recognized by a Bxb1 recombinase. In some embodiments, the RSS is an attP sequence recognized by a Bxb1 recombinase.

In some embodiments, the recombinase is Pa01. In some embodiments, the RSS is an attB sequence recognized by a Pa01 recombinase. In some embodiments, the RSS is an attP sequence recognized by a Pa01 recombinase.

The DNA polynucleotide can be in any configuration that allows for recombination mediated by the recombinase and the RSSs. For example, the DNA polynucleotide can be a plasmid, a circular construct, or a minicircle.

The donor sequence can be any sequence for targeted integration into the TRAC gene. In some embodiments, the donor sequence comprises an expression cassette comprising a gene or an open reading frame (ORF) driven by one or more promoters. In some embodiments, the donor sequence comprises multiple expression cassettes each driven by one or more promoters. In some embodiments, the donor sequence comprises an expression cassette comprising two or more genes or ORFs driven by a promoter, wherein the two or more genes or ORFs are separated by a sequence encoding a self-cleaving peptide, e.g., a 2A peptide. In some embodiments, the donor sequence does not contain a promoter, but contains a splice acceptor sequence preceding the start codon of the transgene ORF. In some embodiments, a transgene or transgene ORF in the donor sequence encodes a polypeptide. In some embodiments, the polypeptide is involved binding, regulation, or activity of an immune cell, e.g., a T cell, and may function in combination or synergistically with disruption of the TRAC gene by prime editing. In some embodiments, the polypeptide is a cellular receptor or a portion thereof, e.g., a receptor that directs specific antigen binding. For example, the polypeptide may be a chimeric antigen receptor (CAR), a T cell receptor (TCR), a B cell receptor, a NK cell receptor, or a functional portion thereof. Exemplary CARs include a CD19 CAR, CD20 CAR, BAFF CAR, B7H2 CAR, GD2 CAR, MSLN CAR, HER2 CAR, EGFR CAR, CD70 CAR, BCMA CAR, or other CARS that are therapeutically relevant. Exemplary TCRs include NY-ESO-1, AHNAK (S2580F), ERBB2 (H473Y), HPV-E6, MAGE-A3/A6, E7, HA-1, or other TCRs that are therapeutically relevant. Exemplary NK cell receptors CD16, NKG2D, DNAM1, NKp46, NKp44, NKp30, LFA1, CD27, or other NK cell receptors that are therapeutically relevant. In some embodiments, the polypeptide is a cellular receptor or a functional portion thereof, e.g., a receptor that modulates immune cell activity. In some embodiments, the polypeptide is a cytokine receptor, e.g., IL2RA, IL2RB, IL7R, IL12R, IL18R, or IFNGR, or a functional portion thereof. In some embodiments, the polypeptide is a chemokine receptor, e.g., CCR2, CCR4, CCR5, CCR6, CXCR1, CXCR2, CXCR3, CXCR4, or a functional portion thereof. In some embodiments, the polypeptide is an immune checkpoint protein, e.g., PD1, CTLA4, CISH, TIGIT, TIM3, or LAG3. In some embodiments, the polypeptide is a cytokine, e.g., IL2, IL15, IL7, TNF, or IFNG. In some embodiments, the polypetide is a chemokine, e.g., CCL2, CCL5, CCL17, CCL20, CCL22, CXCL2, CXCL5, CXCL6, CXCL9, CXCL10, CXCL11, or CXCL12. In some embodiments, the polypeptide is a dominant-negative mutant of a protein involved in immune cell signaling, e.g. T cell signaling. For example, the polypeptide may be a dominant negative mutant of TGFBR, IL10R, IL35R, FOXP3, or STAT5. In some embodiments, the polypeptide is a transcription factor involved in immune cell signaling, e.g., T cell signaling. For example, the polypeptide may be BLIMP1, IL2RA, IL2RB, BCL6, ID2, C-MYB, IL6R, TCF1, EOMES, STAT1, STAT3, STAT4, STAT5, TBET, NFKB, GATA-3, IRF-4. In some embodiments, the polypeptide may be a signaling protein, e.g., LCK, SYK, or ZAP70. In some embodiments, the polypeptide is a reporter protein, e.g., a fluorescence protein. In some embodiments, the polypeptide is an epitope tag. In some embodiments, the polypeptide is a “suicide protein” or a “kill switch” that allows for specific elimination of cells containing the polypeptide, e.g., for specific elimination of edited cells after administration. For example, the polypeptide may be iCasp9, HSV-TK, or an epitope for antibody-mediated elimination (e.g., CD20 for rituximab-mediated elimination).

The recombinase (or polynucleotides encoding the recombinase) and/or the DNA polynucleotide may be provided to a cell containing the target TRAC gene simultaneously or sequentially. In some embodiments, the recombinase or polynucleotides encoding the recombinase and/or the DNA polynucleotide may be provided 1, 2, 3, 4, 5, 6, 7, 8, 9, 10 or more days after contacting the cell with a prime editor (or polynucleotide encoding the prime editor) and the PEgRNAs. In some embodiments, the recombinase or polynucleotides encoding the recombinase and/or the DNA polynucleotide may be provided at the same time a prime editor (or polynucleotide encoding the prime editor) and the PEgRNAs. In some embodiments, the recombinase is fused to, or connected to via a linker, e.g., a peptide linker, the prime editor, e.g., a prime editor fusion protein. The prime editor or any component thereof and the recombinase, can be encoded by DNA vectors (e.g. AAV vectors) or mRNAs.

In one aspect, provided herein is a prime editing system or composition comprising (a) a prime editing guide RNA (PEgRNA) or one or more polynucleotides encoding the PEgRNA, wherein the PEgRNA comprises: (i) a spacer that is complementary to a search target sequence on a first strand of a target gene, (ii) a gRNA core capable of binding to a Cas9 nickase, and (iii) an extension arm comprising a primer binding site (PBS) that comprises a region of complementarity to a second strand of the target gene and an editing template encoding a recombinase recognition sequence (RSS) recognized by a Pa01 recombinase; (b) a prime editor comprising the Cas9 nickase capable of nicking the second strand of the target gene at a nick site and a reverse transcriptase, or one or more polynucleotides encoding the prime editor; and (c) a Pa01 recombinase or one or more polynucleotides encoding the Pa01 recombinase.

In some embodiments, the PBS comprises a region of complementarity to a region upstream of the nick site. In some embodiments, the editing template comprises a region of complementarity to a region downstream of the nick site. In some embodiments, the Cas9 nickase comprises a nuclease inactivation mutation in a HNH domain.

In one aspect, provided herein is a prime editing system or composition comprising (A) a first prime editing guide RNA (PEgRNA) or one or more polynucleotides encoding the first PEgRNA, (B) a second PEgRNA or one or more polynucleotides encoding the second PEgRNA, (C) a prime editor comprising a Cas9 nickase having a nuclease inactivation mutation in a HNH domain and a reverse transcriptase, or one or more polynucleotides encoding the prime editor, and (D) a Pa01 recombinase or one or more polynucleotides encoding the Pa01 recombinase, wherein the first PEgRNA comprises: (i) a first spacer that is complementary to a first search target sequence on a first strand of a target gene, (ii) a first gRNA core capable of binding to the Cas9 nickase; and (iii) a first extension arm comprising a first editing template and a first primer binding site (PBS), wherein the second PEgRNA comprises: (i) a second spacer that is complementary to a second search target sequence on a second strand of the target gene complementary to the first strand, (ii) a second gRNA core capable of binding to the Cas9 nickase; and (iii) a second extension arm comprising a second editing template and a second PBS, wherein the first editing template comprises a region of complementarity to the second editing template, wherein the first editing template and the second editing template each encodes all or a fragment of a recombinase recognition sequence (RSS) or the reverse complement thereof, wherein the first editing template encodes at least a 5′ portion of the RSS or the reverse complement thereof, wherein the second editing template encodes at least a 3′ portion of the RSS or the reverse complement thereof, wherein at least 10 nucleotides at the 5′ end of the first and the second editing templates have perfect reverse complementarity to each other, and wherein the RSS is recognized by the Pa01 recombinase.

In some embodiments, at least 15, 20, 25, or 30 nucleotides at the 5′ end of the first and second editing templates have perfect reverse complementarity to each other. In some embodiments, the first editing template encodes the RSS. In some embodiments, the second editing template encodes the RSS.

In some embodiments, the first editing template comprises a 5′ fragment of an RTT listed in RTT pair 2 or 3 of Table 6 and wherein the second editing template comprises a full length or 5′ fragment of the corresponding RTT pair and wherein at least 10 nucleotides at the 5′ end of the first and second editing templates have perfect reverse complementarity to each other. In some embodiments, the second editing template comprises a 5′ fragment of an RTT listed in RTT pair 2 or 3 of Table 6 and wherein the first editing template comprises a full length or 5′ fragment of the corresponding RTT pair and wherein at least 10 nucleotides at the 5′ end of the first and second editing templates have perfect reverse complementarity to each other.

In some embodiments, the first PBS comprises a region of complementarity to a region upstream of a nick site in the second strand, and wherein the second PBS comprises a region of complementarity to a region upstream of a nick site in the first strand.

In some embodiments, the prime editing system or composition further comprises a DNA polynucleotide that comprises (i) a second RSS recognized by the Pa01 recombinase, and (ii) a donor sequence. In some embodiments, in any one of the prime editing compositions disclosed herein, the target gene is TRAC.

### Methods of Editing TRAC in Immune Cells

The methods, systems, and compositions disclosed herein can be used to edit a target gene of interest by dual prime editing.

In some embodiments, the prime editing method comprises contacting a target gene, e.g., a TRAC gene, with a PERNA and a prime editor (PE) polypeptide or a polynucleotide encoding the PE polypeptide described herein. In some embodiments, the dual prime editing method comprises contacting a target gene, e.g., a TRAC gene, with a first PEgRNA, a second PEgRNA and a PE polypeptide or a polynucleotide encoding the PE polypeptide described herein. In some embodiments, the target gene is double-stranded, and comprises two strands of DNA complementary to each other. In some embodiments, the contacting with the two PEgRNAs and the contacting with a prime editor are performed sequentially. In some embodiments, the contacting with a prime editor is performed after the contacting with the two PEgRNAs. In some embodiments, the contacting with the two PEgRNAs is performed after the contacting with a prime editor. In some embodiments, the contacting with the two PEgRNAs is performed simultaneously either prior to or after contacting with a prime editor. In some embodiments, the contacting with the two PERNAs is performed sequentially either prior to or after contacting with a prime editor. In some embodiments, the contacting with the two PEgRNAs, and the contacting with a prime editor are performed simultaneously. In some embodiments, the two PEgRNAs and the prime editor are associated in complexes prior to contacting a target gene.

In some embodiments, contacting the target gene with the prime editing composition results in binding of the first PERNA to a first strand of the target gene, e.g., a TRAC gene. In some embodiments, contacting the target gene with the prime editing composition results in binding of the second PEgRNA to a second strand of the target gene, e.g., a TRAC gene. In some embodiments, contacting the target gene with the prime editing composition results in binding of the first PEgRNA to a first strand of the target gene and binding of the second PEgRNA to a second strand of the target gene, e.g., a TRAC gene. In some embodiments, contacting the target gene with the prime editing composition results in binding of the first PERNA to a first search target sequence on the first strand of the target gene upon contacting with the first PEgRNA. In some embodiments, contacting the target gene with the prime editing composition results in binding of the second PEgRNA to a second search target sequence on the second strand of the target gene upon contacting with the second PERNA. In some embodiments, contacting the target gene with the prime editing composition results in binding of the first PEgRNA to a first search target sequence on the first strand of the target gene upon contacting with the first PEgRNA and binding of the second PEgRNA to a second search target sequence on the second strand of the target gene upon contacting with the second PEgRNA. In some embodiments, contacting the target gene with the prime editing composition results in binding of a first spacer of the first PEgRNA to a first search target sequence on the first strand of the target gene upon said contacting of the first PEgRNA. In some embodiments, contacting the target gene with the prime editing composition results in binding of a second spacer of the second PEgRNA to a second search target sequence on the second strand of the target gene upon said contacting of the second PEgRNA. In some embodiments, contacting the target gene with the prime editing composition results in binding of a first spacer of the first PEgRNA to a first search target sequence on the first strand of the target gene upon said contacting of the first PERNA and binding of a second spacer of the second PEgRNA to a second search target sequence on the second strand of the target gene upon said contacting of the second PEgRNA.

In some embodiments, contacting the target gene with the prime editing composition results in binding of the prime editor to the target gene, e.g., the target TRAC gene, upon the contacting of the PE composition with the target gene. In some embodiments, a DNA binding domain of a prime editor associates with either a first PEgRNA and/or a second PERNA. In some embodiments, a prime editor associated with a first PEgRNA binds the first strand of a target gene, e.g., a TRAC gene, as directed by the first PEgRNA. In some embodiments, a prime editor associated with a second PEgRNA binds the second strand of a target gene, e.g., a TRAC gene, as directed by the second PEgRNA. In some embodiments, a prime editor associated with a first PEgRNA binds the first strand of a target gene as directed by the first PEgRNA, and a prime editor associated with a second PEgRNA binds the second strand of the target gene as directed by the second PEgRNA.

In some embodiments, a first PEgRNA directs a prime editor to generate a nick on the second strand of a target gene. In some embodiments, a second PEgRNA directs a prime editor to generate a nick on the first strand of a target gene. In some embodiments, a first PEgRNA directs a prime editor to generate a first nick on the second strand of a target gene, and a second PEgRNA directs a prime editor to generate a second nick on the first strand of a target gene, thereby generating an inter-nick duplex (IND) between the position of the first nick and the position of the second nick on the target gene. In some embodiments, the DNA binding domain of the prime editor is a Cas domain. In some embodiments, the DNA binding domain of the prime editor is a Cas9. In some embodiments, the DNA binding domain of the prime editor is a Cas9 nickase.

In some embodiments, contacting the target gene with the prime editing composition results in hybridization of the PEgRNA (e.g., the first PEgRNA and/or the second PEgRNA) with the 3′ end of the nicked single-stranded DNA, thereby priming DNA polymerization by a DNA polymerase domain of the prime editor. In some embodiments, the free 3′ end of the single-stranded DNA generated at the nick site hybridizes to a primer binding site sequence (PBS) of the contacted PEgRNA, thereby priming DNA polymerization. In some embodiments, the DNA polymerization is reverse transcription catalyzed by a reverse transcriptase domain of the prime editor. In some embodiments, the method comprises contacting the target gene with a DNA polymerase, e.g., a reverse transcriptase, as a part of a prime editor fusion protein or prime editing complex (in cis), or as a separate protein (in trans).

In some embodiments, contacting the target gene with the prime editing composition generates an overlap duplex (OD) or replacement duplex (RD) that replaces the IND. In some embodiments, the OD or RD comprises one or more intended nucleotide edits compared to the endogenous sequence of the target gene, e.g., a TRAC gene. In some embodiments, the intended nucleotide edits are incorporated in the target gene by replacement of the IND by the OD or RD. In some embodiments, the intended nucleotide edits are incorporated in the target gene by excision of the IND and DNA repair.

In some embodiments, incorporation of the intended nucleotide edits in a TRAC gene results in disruption of the TRAC gene that reduces or abolishes expression of the gene. For example, in some embodiments, the intended nucleotide edits comprise an insertion in the TRAC gene that disrupts the reading frame of the gene. In some embodiments, the intended nucleotide edits comprise an insertion that includes one or more stop codons. In some embodiments, the intended nucleotide edits comprise an insertion of one or more recombinase recognition sequences (RSSs), wherein the insertion disrupts the reading frame of the TRAC gene.

In some embodiments, editing of a TRAC gene further comprises contacting the TRAC gene with one or more recombinases or one or more polynucleotides encoding the recombinases, wherein the PEgRNA or PERNA pair of the prime editing composition encode insertion of one or more recombinase recognition sequences (RSSs) in TRAC gene. In some embodiments, editing of a TRAC gene further comprises contacting the TRAC gene with a DNA polynucleotide for recombination with TRAC containing the RSS(s) introduced by prime editing. In some embodiments, the DNA polynucleotide comprises (a) a donor sequence and (b) one or more RSSs recognized by the recombinase.

In some aspect, disclosed herein is a method for integrating a donor sequence in a target gene, wherein the method comprises contacting the target gene with any one of the prime editing systems or compositions described herein.

Contacting a target gene, e.g., the TRAC gene, with the prime editor (or polynucleotides encoding the prime editor), PERNAs, recombinases (or polynucleotides encoding the recombinases), and optionally the DNA polynucleotide can result in inversion, deletion, insertion of or replacement with an exogenous donor sequence, and other recombination events.

For example, in some embodiments, a method of targeted insertion of an exogenous donor sequence into the TRAC gene comprises contacting the TRAC gene with (a) a PEgRNA or a pair of PEgRNAs capable of inserting a first RSS in the TRAC gene, or one or more polynucleotides encoding the PERNA, (b) a prime editor or one or more polynucleotides encoding the prime editor, (c) a recombinase that recognizes the first RSS, or one or more polynucleotides encoding the recombinase, and (d) a DNA polynucleotide comprising (i) a second RSS recognized by the recombinase and (ii) a donor sequence. Recombination between the first RSS and the second RSS can result in insertion of the donor sequence at the first RSS in the TRAC gene.

In some embodiments, a method of targeted replacement of an endogenous TRAC sequence with an exogenous donor sequence comprises contacting the TRAC gene with (a) a PEgRNA or a pair of PEgRNAs capable of inserting a first RSS and a second RSS in the TRAC gene, or one or more polynucleotides encoding the PEgRNA, (b) a prime editor or one or more polynucleotides encoding the prime editor, (c) a recombinase that recognizes the first RSS, or one or more polynucleotides encoding the recombinase, and (d) a DNA polynucleotide comprising (i) a third RSS and a fourth RSS recognized by the recombinase and (ii) a donor sequence between the third and the fourth RSSs. Recombination between the RSSs in the TRAC gene and the RSSs in the DNA polynucleotide can result in insertion of the donor sequence at the first RSS in the TRAC gene.

In some embodiments, a method of targeted deletion of an endogenous sequence in the TRAC gene comprises contacting the TRAC (a) a PEgRNA or a pair of PERNAs capable of inserting a first RSS and a second RSS in the TRAC gene, or one or more polynucleotides encoding the PEgRNA, (b) a prime editor or one or more polynucleotides encoding the prime editor, and (c) a recombinase that recognizes the first and the second RSS. Recombination between the first RSS and the second RSS can result in deletion of the TRAC gene sequence between the two RSSs.

In some embodiments, a method of introducing a targeted inversion of an endogenous sequence in the TRAC gene comprises contacting the TRAC (a) a PEgRNA or a pair of PEgRNAs capable of inserting a first RSS and a second RSS in the TRAC gene, or one or more polynucleotides encoding the PEgRNA, (b) a prime editor or one or more polynucleotides encoding the prime editor, and (c) a recombinase that recognizes the first and the second RSS. Recombination between the first RSS and the second RSS can result in inversion of the TRAC gene sequence between the two RSSs.

In some embodiments, a method of targeted insertion of an exogenous donor sequence into the TRAC gene comprises contacting the TRAC gene with (a) a first PEgRNA comprising (i) a first spacer that is complementary to first search target sequence on a first strand of the TRAC gene, (ii) a first gRNA core capable of binding to a Cas9 protein, and (iii) a first extension arm comprising a first editing template and a first primer binding site (PBS), or one or more polynucleotides encoding the first PEgRNA, (b) a second PEgRNA comprising (i) a second spacer that is complementary to second search target sequence on a second strand of the TRAC gene, (ii) a second gRNA core capable of binding to a Cas9 protein, and (iii) a second extension arm comprising a second editing template and a second primer binding site (PBS), or one or more polynucleotides encoding the first PEgRNA, (c) a prime editor or one or more polynucleotides encoding the prime editor, (d) a recombinase or one or more polynucleotides encoding the recombinase, and (e) a DNA polynucleotide, wherein the first editing template and the second editing template comprise a region of complementarity to each other, wherein the first editing template connected at its 5′ end to the reverse complement of the non-complementary region of the second editing template comprises a first RSS recognized by the recombinase, and wherein the DNA polynucleotide comprises (i) a second RSS recognized by the recombinase and (ii) a donor sequence. In some embodiments, recombination between the first RSS and the second RSS results in integration of the donor sequence at the first RSS in the TRAC gene. In some embodiments, the first PEgRNA comprises a spacer selected from any one of the 5′ spacer sequences of Tables 9-12, and the first PBS comprises a PBS sequence selected from the same table as the first spacer. In some embodiments, the second PEgRNA comprises a spacer selected from any one of the 3′ spacer sequences of Tables 13-25, and the second PBS comprises a PBS sequence selected from the same table as the second spacer. In some embodiments, the first editing template encodes a first single stranded DNA, the second editing template encodes a second single stranded DNA, and incorporation of the first and second single stranded DNA in the TRAC gene via dual prime editing results in replacement of the IND with a replacement duplex (RD) based on sequences of the first and second single stranded DNA, wherein the RD contains the first RSS sequence. In some embodiments, the first editing template encodes the first RSS sequence. In some embodiments, the second editing template encodes the first RSS sequence. The first and second RSS sequences and the recombinase can be any RSS and corresponding recombinases known in the art. In some embodiments, the first RSS is an attB sequence, and the second RSS is an attP sequence, or vice versa. In some embodiments, the recombinase is Bxb1. In some embodiments, the first editing template comprises a RTT #1 sequence in Table 6 having the RTT Pair No. 1 and the second editing template comprises a RTT #2 sequence in Table 6 having the same RTT Pair No. In some embodiments, the first editing template comprises a RTT #2 sequence in Table 6 having the RTT Pair No. 1 and the second editing template comprises a RTT #1 sequence in Table 6 having the same RTT Pair No. In some embodiments, the recombinase is Pa01. In some embodiments, the first editing template comprises a RTT #1 sequence in Table 6 having the RTT Pair No. 2 or 3 and the second editing template comprises a RTT #2 sequence in Table 6 having the same RTT Pair No. In some embodiments, the first editing template comprises a RTT #2 sequence in Table 6 having the RTT Pair No. 2 or 3 and the second editing template comprises a RTT #1 sequence in Table 6 having the same RTT Pair No. In some embodiments, the first editing template comprises a 5′ fragment of an RTT listed in Table 6 and wherein the second editing template comprises a full length or 5′ fragment of the corresponding RTT pair and wherein at least 10 nucleotides at the 5′ end of the first and second editing templates have perfect reverse complementarity to each other. In some embodiments, the second editing template comprises a 5′ fragment of an RTT listed in Table 6 and wherein the first editing template comprises a full length or 5′ fragment of the corresponding RTT pair and wherein at least 10 nucleotides at the 5′ end of the first and second editing templates have perfect reverse complementarity to each other.

Additional exemplary RSS sequences, including recombination RSS pairs (e.g. attB and attP sequences) and corresponding recombinases are provided in Tables 5 and 27.

In some embodiments, the first PERNA contains a PEgRNA sequence selected from any one of Tables 9-12, and the second PERNA contains a PERNA sequence selected from any one of Tables 13-25, wherein the second PEgRNA comprises an editing template that has the same RTT Pair Number as the first PEgRNA.

The DNA polynucleotide can be any suitable sequence or configuration, e.g., a plasmid or a minicircle. The donor sequence in the DNA polynucleotide can be any sequence for targeted integration into the TRAC gene. For example, the donor sequence can comprise one or more expression cassettes containing one or more promoters. In some embodiments, the donor sequence comprises sequences that encode one or more polypeptides. In some embodiments, the donor sequence comprises one or more open reading frames (ORFs) that encode one or more polypeptides. In some embodiments, the polypeptide(s) can comprise a cellular receptor, an immune checkpoint protein, a cytokine, a chemokine, a dominant negative mutant of a protein involved in immune cell signaling, a transcription factor, a signaling protein, a reporter protein, an epitope tag, a kill switch, or any protein or functional portion or fragment thereof for integration into the TRAC gene as described herein or known in the art.

Contacting with the PEgRNAs or polynucleotide(s) encoding same, the prime editor or polynucleotide(s) encoding same, the recombinase or polynucleotide(s) encoding same, and the DNA polynucleotide may be performed simultaneously or sequentially. In some embodiments, the method of editing the TRAC gene comprises contacting the TRAC gene with the PEgRNAs or polynucleotide(s) encoding same, the prime editor or polynucleotide(s) encoding same followed by contacting the TRAC gene with the recombinase or polynucleotide(s) encoding same, and the DNA polynucleotide. In some embodiments, the recombinase or polynucleotides encoding the recombinase and/or the DNA polynucleotide may be provided 1, 2, 3, 4, 5, 6, 7, 8, 9, or 10 days after contacting the TRAC (e.g., by introducing into a cell containing the TRAC) with a prime editor (or polynucleotide encoding the prime editor) and the PERNAs. In some embodiments, the recombinase or polynucleotides encoding the recombinase and/or the DNA polynucleotide may be provided at the same time a prime editor (or polynucleotide encoding the prime editor) and the PEgRNAs.

In some embodiments, the target TRAC gene is a wild-type TRAC gene. In some embodiments, the target TRAC gene comprises one or more mutations compared to a wild-type TRAC sequence.

In some embodiments, the target gene, e.g., a TRAC gene, is in a cell. Accordingly, also provided herein are methods of modifying, engineering, or editing a cell, such as a human cell, a human primary cell, a human iPSC-derived cell, and/or a human T-cell or a precursor thereof, or a progenitor thereof.

In some embodiments, the prime editing method comprises introducing a first PEgRNA, a second PERNA, and a prime editor into the cell that has the target gene. In some embodiments, the method comprises further introducing into the cell a recombinase or one or more polynucleotide encoding the recombinase and optionally a DNA polynucleotide comprising one or more RSSs recognized by the recombinase and a donor sequence. In some embodiments, the prime editing method comprises introducing into the cell that has the target gene with a prime editing composition comprising a first PEgRNA, a second PEgRNA, and a prime editor polypeptide. In some embodiments, the first PERNA, the second PEgRNA, and the prime editor polypeptides form complexes prior to the introduction into the cell. In some embodiments, the first PERNA, the second PEgRNA, and the prime editor polypeptides form complexes after the introduction into the cell. In some embodiments, the recombinase is fused, linked, or otherwise complexed with the prime editor polypeptide.

The prime editors, PEgRNAs, recombinases, DNA polynucleotides containing donor sequences, and prime editing complexes may be introduced into the cell by any delivery approaches described herein or any delivery approach known in the art, including ribonucleoprotein (RNPs), lipid nanoparticles (LNPs), viral vectors, non-viral vectors, mRNA delivery, and physical techniques such as cell membrane disruption by a microfluidics device. The prime editors, PEgRNAs, and prime editing complexes may be introduced into the cell simultaneously or sequentially.

In some embodiments, the prime editing method comprises introducing into the cell a first PEgRNA and a second PEgRNA, or polynucleotides encoding the first PEgRNA and the second PEgRNA, and a prime editor polynucleotide encoding a prime editor polypeptide. In some embodiments, the method comprises introducing the first PEgRNA and the second PEgRNA or the polynucleotides encoding the first PEgRNA and the second PERNA, and the polynucleotide encoding the prime editor polypeptide into the cell simultaneously. In some embodiments, the method comprises introducing the first PEgRNA and the second PEgRNA or the polynucleotides encoding the first PEgRNA and the second PEgRNA, and the polynucleotide encoding the prime editor polypeptide into the cell sequentially. In some embodiments, the method comprises introducing the polynucleotide encoding the prime editor polypeptide into the cell before introduction of the first PERNA and the second PEgRNA or the polynucleotides encoding the first PEgRNA and the second PEgRNA. In some embodiments, the polynucleotide encoding the prime editor polypeptide is introduced into and expressed in the cell before introduction of the first PERNA and the second PEgRNA or the polynucleotides encoding the first PEgRNA and the second PEgRNA into the cell. In some embodiments, the polynucleotide encoding the prime editor polypeptide is introduced into the cell after the first PEgRNA and the second PEgRNA or the polynucleotides encoding the first PEgRNA and the second PEgRNA are introduced into the cell. In some embodiments, the method further comprises introducing into the cell a recombinase or one or more polynucleotides encoding the recombinase. In some embodiments, the method further comprises introducing into the cell a DNA polynucleotide comprising one or more RSSs recognized by the recombinase and a donor sequence. In some embodiments, the recombinase and/or the one or more polynucleotides encoding the recombinase are introduced after introduction of first PEgRNA, second PEgRNA, prime editor, or the one or more polynucleotides encoding the same. In some embodiments, the recombinase and/or the one or more polynucleotides encoding the recombinase are introduced 1, 2, 3, 4, 5, 6, 7, 8, 9, 10 or more days after introduction of first PEgRNA, second PEgRNA, prime editor, or the one or more polynucleotides encoding the same. In some embodiments, the one or more polynucleotides encoding the PEgRNAs, the one or more polynucleotides encoding the prime editor, and/or the one or more polynucleotides encoding the recombinase are in DNA vectors, e.g., AAV vectors. In some embodiments, one or more polynucleotides encoding the prime editor and/or the one or more polynucleotides encoding the recombinase are mRNA. The one or more polynucleotide encoding the prime editor polypeptide, the first PEgRNA and the second PEgRNA or the polynucleotides encoding the first PEgRNA and the second PERNA, the recombinase or the one or more polynucleotides encoding the recombinase, and the DNA polynucleotide may be introduced into the cell by any delivery approaches described herein or any delivery approach known in the art, for example, by RNPs, LNPs, viral vectors, non-viral vectors, mRNA delivery, and physical delivery.

In some embodiments, the polynucleotide encoding the prime editor polypeptide and the polynucleotides encoding the first PEgRNA and the second PEgRNA integrate into the genome of the cell after being introduced into the cell. In some embodiments, the polynucleotide encoding the prime editor polypeptide and the polynucleotides encoding the first PEgRNA and the second PERNA are introduced into the cell for transient expression. In some embodiments, the one or more polynucleotides encoding the recombinase are introduced into the cell for transient expression.

Accordingly, also provided herein are cells modified by prime editing.

In some embodiments, the cell is a mammalian cell. In some embodiments, the cell is a non-human primate cell, bovine cell, porcine cell, rodent, or mouse cell. In some embodiments, the cell is a human cell. In some embodiments, the edited cell (or the population of edited cells) is a mammalian cell. In some embodiments, the edited cell is a human cell. In some embodiments, the cell is an immune cell (e.g., a primary immune cell) or a progenitor or a precursor thereof). In some embodiments, the cell is a T cell, a B cell, a NK cell, or a progenitor or a precursor thereof.

In some embodiments, the cell is a primary cell. In some embodiments, the cell is a human primary cell.

In some embodiments, the target gene, e.g., the TRAC gene, edited by prime editing is in a chromosome of the cell. In some embodiments, the intended nucleotide edits incorporate in the chromosome of the cell and are inheritable by progeny cells. In some embodiments, the intended nucleotide edits introduced to the cell by the prime editing systems, compositions, and methods are such that the cell and progeny of the cell also include the intended nucleotide edits. In some embodiments, the cell is autologous, allogeneic, or xenogeneic to a subject. In some embodiments, the cell is from or derived from a subject. In some embodiments, the cell is from or derived from a human subject. In some embodiments, the cell is introduced back into the subject, e.g., a human subject, after incorporation of the intended nucleotide edits by prime editing.

In some embodiments, the method provided herein comprises introducing the prime editor polypeptide or the polynucleotide encoding the prime editor polypeptide, and the PEgRNAs (e.g., the first PEgRNA and the second PEgRNA) or the polynucleotides encoding the PEgRNAs, into a plurality or a population of cells that comprise the target gene. In some embodiments, the population of cells is of the same cell type. In some embodiments, the population of cells is of the same tissue or organ. In some embodiments, the population of cells is heterogeneous. In some embodiments, the population of cells is homogeneous. In some embodiments, the population of cells is from a single tissue or organ, and the cells are heterogeneous. In some embodiments, the introduction into the population of cells is ex vivo. In some embodiments, the introduction into the population of cells is in vivo, e.g., into a human subject.

In some embodiments, the target gene, e.g., the TRAC gene, is in a genome of each cell of the population. In some embodiments, introduction of the prime editor polypeptide or the polynucleotide encoding the prime editor polypeptide, and the PEgRNAs (e.g., the first PEgRNA and the second PERNA) or the polynucleotides encoding the PEgRNAs, results in incorporation of one or more intended nucleotide edits in the target gene in at least one of the cells in the population of cells. In some embodiments, introduction of the prime editor polypeptide or the polynucleotide encoding the prime editor polypeptide, and the PEgRNAs or the polynucleotides encoding the PERNAs, results in incorporation of the one or more intended nucleotide edits in the target gene in a plurality of the population of cells. In some embodiments, introduction of the prime editor polypeptide or the polynucleotide encoding the prime editor polypeptide, and the PEgRNAs or the polynucleotide encoding the PEgRNAs, results in incorporation of the one or more intended nucleotide edits in the target gene in each cell of the population of cells. In some embodiments, incorporation of the intended nucleotide edits in a TRAC gene in a plurality or population of cells results in disruption of the TRAC gene that reduces or abolishes expression of the gene. For example, in some embodiments, the intended nucleotide edits comprise an insertion in the TRAC gene that disrupts the reading frame of the gene. In some embodiments, the intended nucleotide edits comprise an insertion that includes one or more stop codons. In some embodiments, the intended nucleotide edits comprise an insertion of one or more recombinase recognition sequences (RSSs), wherein the insertion disrupts the reading frame of the TRAC gene. In some embodiments, incorporation of the intended nucleotide edits results in disruption of the TRAC gene and reduced or abolished expression of the TRAC gene in a fraction of the population of cells or in each cell of the population of cells.

In some embodiments, the method provided herein further comprises introducing a recombinase or one or more polynucleotides encoding the recombinase, and optionally a DNA polynucleotide comprising one or more RSSs recognized by the recombinase and a donor sequence into a plurality or a population of cells that comprise the TRAC gene. Introduction of the recombinase or the polynucleotide(s) encoding the recombinase and the DNA polynucleotide can be performed simultaneously or sequentially (e.g. after) introduction of the prime editor or polynucleotide(s) encoding the prime editor and the PEgRNAs or polynucleotides encoding the PEgRNAs. In some embodiments, introduction of the recombinase or one or more polynucleotides encoding the recombinase the DNA polynucleotide results in integration of the donor sequence into the TRAC gene.

Disruption of the TRAC gene and reduced/abolished expression of the CD3 protein may be therapeutically relevant in immune therapy. For example, immune therapy using allogeneic immune cells, e.g., T cells from healthy donors, are advantageous compared to autologous cell therapy in terms of manufacturing time, cost, feasibility, and scalability, but have limitations such as off-tumor toxicity and graft-versus-host-disease (GvHD). Reduced or abolished expression of CD3 disrupts the endogenous T cell receptor (TCR) complex, which may lead to reduced GvHD and allow for wide clinical application of allogeneic T cells.

In addition, in some embodiments, integration of the donor sequence into the TRAC gene results in expression of a polypeptide encoded by the donor sequence. The donor sequence can comprise any transgene or open reading frame (ORF) that is therapeutically relevant. In some embodiments, the donor sequence comprises a sequence, e.g., an ORF that encodes a polypeptide that is involved in signaling, binding, regulation, or activity of an immune cell, e.g., a T cell. For example, the polypeptide may be a cellular receptor, an immune checkpoint protein, a cytokine, a chemokine, a dominant negative mutant of a protein involved in immune cell signaling, a transcription factor, a signaling protein, a reporter protein, an epitope tag, a kill switch, or any protein or functional portion or fraction thereof. The polypeptide may thus function to direct the immune cell to a specific target, e.g., a tumor cell, activate the immune cell, reduce tumor inhibition of immune response, regulate immune cell activity, or selectively eliminate certain immune cells. The polypeptides encoded by the donor sequence in combination with disruption of the TRAC gene can be used for generation of allogeneic immune cells, e.g., T cells, having improved target specificity (e.g., tumor specificity mediated by CARs or surface receptors), improved activity, reduced GvHD effect, and/or regulation potential in immune therapy.

Accordingly, provided herein are methods of editing a cell or population of cells and cells edited by prime editing. Also provided herein are pharmaceutical compositions comprising a prime editing composition described herein, for example, a pharmaceutical composition comprising a LNP comprising a prime editing composition (or components thereof) described herein. Also provided herein are method of making a pharmaceutical composition comprising a prime edited cell or a population of prime edited cells. Also provided herein are pharmaceutical compositions comprising a prime edited cell or a population of prime edited cells. Also provided herein are methods for treating a disease, e.g., a cancer, in a subject in need thereof, wherein the method comprises administering the pharmaceutical compositing a prime edited cell or a population of prime edited cells to the subject.

In one aspect, provided herein are an isolated cell or a population of cells (e.g., an isolated T cell or a population of T cells) generated by the prime editing methods disclosed herein.

In some embodiments, the edited cell or population of cells comprise a disruption in the TRAC gene, wherein the disruption reduces or abolishes expression or function of the CD3 protein. In some embodiments, the edited cell or population of cells comprises an exogenous sequence (i.e. the integrated donor sequence) in the TRAC gene. In some embodiments, the exogenous sequence comprises an expression cassette comprising a gene or an open reading frame (ORF) driven by one or more promoters. In some embodiments, the exogenous sequence comprises multiple expression cassettes each driven by one or more promoters. In some embodiments, the exogenous sequence comprises an expression cassette comprising two or more genes or ORFs driven by a promoter, wherein the two or more genes or ORFs are separated by a sequence encoding a self-cleaving peptide, e.g., a 2A peptide. In some embodiments, the exogenous sequence does not contain a promoter, but contains a splice acceptor sequence preceding the start codon of the transgene ORF. In some embodiments, a transgene or transgene ORF in the donor sequence encodes a polypeptide. In some embodiments, the polypeptide is involved binding, regulation, or activity of an immune cell, e.g., a T cell, and may function in combination or synergistically with disruption of the TRAC gene by prime editing. In some embodiments, the polypeptide is a cellular receptor or a portion thereof, e.g., a receptor that directs specific antigen binding. For example, the polypeptide may be a chimeric antigen receptor (CAR), a T cell receptor (TCR), a B cell receptor, a NK cell receptor, or a functional portion thereof. In some embodiments, the CAR has specificity for a target antigen that is a disease associated antigen. In some embodiments, the CAR specifically binds a target antigen or a neoantigen expressed by a cancer cell (e.g., expressed or presented on the surface of a cancer cell). In some embodiments, the target antigen or a neoantigen is from an oncogene or tumor suppressor gene (e.g., a mutated tumor suppressor gene). In some embodiments, the target antigen comprises a T cell epitope. In some embodiments, the target antigen recognized and bound by the extracellular domain is a protein or peptide, a nucleic acid, a lipid, or a polysaccharide. In some embodiments, the target antigen is autologous, and is expressed on a diseased or otherwise altered cell. For example, in some embodiments, the target antigen is expressed in a neoplastic cell. In some embodiments, the CAR or cellular receptor specifically binds to a target antigen expressed by a pathogen e.g., a bacterium, virus, fungus, yeast, parasite (e.g., single-celled or multicellular eukaryotic parasite), or other microorganism. In some embodiments, the cellular receptor or CAR specifically binds to a target antigen associated with a disease (e.g., an inflammatory or autoimmune disease). Exemplary CARs include a CD19 CAR, CD20 CAR, BAFF CAR, B7H2 CAR, GD2 CAR, MSLN CAR, HER2 CAR, EGFR CAR, CD70 CAR, BCMA CAR, or other CARs that are therapeutically relevant. Exemplary TCRs include NY-ESO-1, AHNAK (S2580F), ERBB2 (H473Y), HPV-E6, MAGE-A3/A6, E7, HA-1, or other TCRs that are therapeutically relevant. Exemplary NK cell receptors CD16, NKG2D, DNAM1, NKp46, NKp44, NKp30, LFA1, CD27, or other NK cell receptors that are therapeutically relevant. In some embodiments, the polypeptide is a cellular receptor or a functional portion thereof, e.g., a receptor that modulates immune cell activity. In some embodiments, the polypeptide is a cytokine receptor, e.g., IL2RA, IL2RB, IL7R, IL12R, IL18R, or IFNGR, or a functional portion thereof. In some embodiments, the polypeptide is a chemokine receptor, e.g., CCR2, CCR4, CCR5, CCR6, CXCR1, CXCR2, CXCR3, CXCR4, or a functional portion thereof. In some embodiments, the polypeptide is an immune checkpoint protein, e.g., PD1, CTLA4, CISH, TIGIT, TIM3, or LAG3. In some embodiments, the polypeptide is a cytokine, e.g., IL2, IL15, IL7, TNF, or IFNG. In some embodiments, the polypetide is a chemokine, e.g., CCL2, CCL5, CCL17, CCL20, CCL22, CXCL2, CXCL5, CXCL6, CXCL9, CXCL10, CXCL11, or CXCL12. In some embodiments, the polypeptide is a dominant-negative mutant of a protein involved in immune cell signaling, e.g. T cell signaling. For example, the polypeptide may be a dominant negative mutant of TGFBR, IL10R, IL35R, FOXP3, or STAT5. In some embodiments, the polypeptide is a transcription factor involved in immune cell signaling, e.g. T cell signaling. For example, the polypeptide may be BLIMP1, IL2RA, IL2RB, BCL6, ID2, C-MYB, IL6R, TCF1, EOMES, STAT1, STAT3, STAT4, STAT5, TBET, NFKB, GATA-3, IRF-4. In some embodiments, the polypeptide may be a signaling protein, e.g., LCK, SYK, or ZAP70. In some embodiments, the polypeptide is a reporter protein, e.g., a fluorescence protein. In some embodiments, the polypeptide is an epitope tag. In some embodiments, the polypeptide is a “suicide protein” or a “kill switch” that allows for specific elimination of cells containing the polypeptide, e.g., for specific elimination of edited cells after administration. For example, the polypeptide may be iCasp9, HSV-TK, or an epitope for antibody-mediated elimination (e.g., CD20 for rituximab-mediated elimination).

In some embodiments, the edited cell or population of cells comprises a sequence in the TRAC gene resulting from recombination between a first RSS in the TRAC gene inserted by prime editing and a second RSS in an exogenous DNA polynucleotide provided. In some embodiments, the first RSS is an attP sequence and the second RSS is an attB sequence. In some embodiments, the sequence resulted from the recombination comprises an attL sequence and an attR sequence. In some embodiments, the edited cell or population of cells are contacted with the recombinase Bxb1. In some embodiments, the edited cell or population of cells comprise an attL sequence GGCTTGTCGACGACGGCGGTCTCAGTGGTGTACGGTACAAACC (SEQ ID NO: 1046) and/or an attR sequence GGTTTGTCTGGTCAACCACCGCGGTCTCCGTCGTCAGGATCAT (SEQ ID NO: 1047).

In some embodiments, the edited cell or population of cells further comprise one or more mutations in an endogenous gene that result in reduced or abolished expression of the endogenous gene. In some embodiments, the edited cell or population of cells further comprises one or more mutations in each of multiple endogenous genes, e.g., 1, 2, 3, 4, 5, 6, 7, 8, 9, 10, or more genes, that result in reduced or abolished expression of the multiple endogenous genes. Mutations in the endogenous gene(s) may be introduced by prime editing or dual prime editing methods as described herein, or by other gene editing approaches such as programmable nuclease mediated knockdown or knockout, e.g., by CRISPR-Cas9 nuclease, zinc finger nuclease, or TALEN designed for specific cleavage of the endogenous gene sequence(s). Such endogenous genes may be involved in specificity, function, regulation, and/or activity of an immune cell. For example, in some embodiments, the endogenous gene encodes a cellular receptor, or a component of a cellular receptor signaling pathway, e.g., other components of the TCR complex. In some embodiments, the endogenous gene encodes a human leukocyte antigen (HLA) component, for example, β2-microglobulin (B2M). Similar to disruption of the TRAC gene, knockout of other TCR complex components or HLA components may also reduce GvHD effect and improve allogeneic immune cells used in cell therapy. In some embodiments, the endogenous gene encodes a checkpoint protein, for example, PDCD1. Knockout of checkpoint proteins may reduce tumor inhibition of immune cell activity, thereby improve on-tumor activity of the immune cells used in cell therapy. In some embodiments, the endogenous genes include CD2, CD3 epsilon, CD3 gamma, CD3 delta, CD4, CD5, CD7, CD30, CD33, CD52, CD70, and CIITA.

In some embodiments, the edited cell or population of cells further comprise one or more transgenes that express one or more functional proteins. The one or more transgenes may be introduced into a specific genomic location by prime editing and recombinase mediated integration methods as described herein, or by other gene editing approaches such as programmable nuclease directed homologous recombination (HDR), e.g., by CRISPR-Cas9 nuclease, zinc finger nuclease, or TALEN combined with HDR donors. The transgenes may also be introduced non-specifically into the cell, e.g., by lentivirus. The transgenes may encode any protein. For example, the polypeptide may be a cellular receptor, an immune checkpoint protein, a cytokine, a chemokine, a dominant negative mutant of a protein involved in immune cell signaling, a transcription factor, a signaling protein, a reporter protein, an epitope tag, a kill switch, or any therapeutic protein or functional portion or fragment thereof.

In some embodiments, the edited cells exhibit reduced the alloreactivity, and may prevent graft vs host disease upon administration to a subject in need. In some embodiments, the edited cells exhibit reduced alloreactivity as compared to a suitable control cell. In some embodiments, administering an effective amount of a composition comprising a population of the edited cells of the disclosure induces a reduced alloreactivity (e.g., by at least about 1%, 2%, 5%, 10%, 20%, 30%, 40%, 50%, 60%, 70%, 80%, 90%, 95%, 99%, or more) to the edited cells in the subject compared to that induced upon administering of composition comprising a population of suitable control cells. In one aspect, provided herein are methods to reduce alloreactivity to a cell, comprising contacting a cell with an effective amount of a prime editing composition disclosed herein. In some embodiments, the edited cells are non-alloreactive cells.

In some embodiments, the edited cell (or the population of edited cells) is a mammalian cell. In some embodiments, the edited cell is a human cell. In some embodiments, the cell is an immune cell (e.g., a primary immune cell) or a progenitor or a precursor thereof). In some embodiments, the cell is a T cell, a B cell, a NK cell, or a progenitor or a precursor thereof. In some embodiments, the cell is a human T cell, or a progenitor or a precursor thereof. In some embodiments, the cell is a T cell, e.g., a primary T cell, e.g., an inflammatory T cell, a T helper cell, a cytotoxic T cell, a CD4+ T cell, a CD8+ T cell, a memory T cell, a regulatory T cell, a natural killer T cell, a mucosal associated invariant T cell, a γδ T cell, an alpha beta T cell, a naive T cell, or an effector T cell), formed elements of the blood (e.g., lymphocytes, bone marrow cells), precursors or progenitors thereof, differentiated or de-differentiated cell thereof, and stem cells. In some embodiments, a cell is a naive T cell (e.g., a naive CD8+ T cell). In some embodiments, the cell is a transformed T cell. In some embodiments, the cell is a memory T cell (e.g., (e.g., central memory T cell (TCM), stem memory T cell (TSCM), effector memory T cell, Tissue resident memory T cell). In some embodiments, the cell is an effector memory T cell (e.g., TEM cells and TEMRA (CD45RA+) cells). In some embodiments, the cell is a regulatory T cell. In some embodiments, the cell is a natural killer T cell. In some embodiments, the cell is a Mucosal associated invariant T cell. In some embodiments, the cell is a γδ T cell. In some embodiments, the cell is an effector T cell. In some embodiments, the cell is a T helper cell (e.g., Th1 cell, Th2 cell, Th9 cell, Th17 cell, Th22 cell, and Tfh (follicular helper) cell). In some embodiments, the cell is a thymocyte. In some embodiments, the cell is a lymphoid cell. In some embodiments, the cell is a common lymphoid progenitor cells. In some embodiments, the cell is an early thymic progenitor cell. In some embodiments, the cell is a CD3+ cell. In some embodiments, the cell is a tumor infiltrating lymphocyte. In some embodiments, the cell is a myeloid cell. In some embodiments, the cell is a plasma cell. In some embodiments, the cell is an activated T cell.

In some embodiments, the cell is a progenitor cell. In some embodiments, the cell is a human progenitor cell. In some embodiments, the cell is a stem cell (e.g., adult stem cell, embryonic stem cell, non-embryonic stem cell), cord blood stem cell, progenitor cell, bone marrow stem cell, induced pluripotent stem cell, totipotent stem cell, a CD34+ cell, or hematopoietic stem cell). In some embodiments, the cell is a pluripotent cell (e.g., a pluripotent stem cell). In some embodiments, the cell (e.g., a stem cell) is an embryonic stem cell, tissue-specific stem cell, mesenchymal stem cell, or an induced pluripotent stem cell. In some embodiments, the cell is an induced pluripotent stem cell (iPSC). In some embodiments, the cell is a hematopoietic stem cell. In some embodiments, the cell is a hematopoietic stem and progenitor cell. In some embodiments, the cell is a multipotent progenitor cell. In some embodiments, the cell is a T-cell progenitor. In some embodiments, the cell is a T-cell precursor. In some embodiments, the cell is an embryonic stem cell (ESC). In some embodiments, the cell is a human stem cell. In some embodiments, the cell is a human pluripotent stem cell. In some embodiments, the cell is a non-embryonic stem cell. In some embodiments, the cell is an induced human pluripotent stem cell. In some embodiments, the cell is a human stem cell. In some embodiments, the cell is a human embryonic stem cell. In some embodiments, the cell is a human T-cell progenitor. In some embodiments, the cell is a human T-cell precursor.

In some embodiments, the cell is not isolated from an organism but forms part of a tissue or organ of an organism, e.g., a mammal.

In some embodiments, the cell is a differentiated cell. In some embodiments, the cell is differentiated from an induced pluripotent stem cell. In some embodiments, the cell is a T-cell e.g., a primary T cell, e.g., an inflammatory T cell, a T helper cell, a cytotoxic T-cell, a CD4+ T-cell, a CD8+ T cell, a memory T cell, a regulatory T cell, a natural killer T cell, a mucosal associated invariant T cell, a γδ T cell, an alpha beta T cell, a naive T cell, or an effector T cell differentiated from an iPSC, ESC, a T-cell precursor, or a T-cell progenitor.

In some embodiments, the cell is a differentiated human cell. In some embodiments, the cell is differentiated from an induced human pluripotent stem cell. In some embodiments, the cell edited by prime editing can be differentiated into, or give rise to recovery of a population of cells, e.g., a T-cell e.g., a primary T cell, e.g., an inflammatory T cell, a T helper cell, a cytotoxic T-cell, a CD4+ T-cell, a CD8+ T cell, a memory T cell, a regulatory T cell, a natural killer T cell, a mucosal associated invariant T cell, a γδ T cell, an alpha beta T cell, a naive T cell, or an effector T cell.

In some embodiments, the cell is in a subject, e.g., a human subject. In some embodiments, the cell is obtained from a subject prior to editing. For example, in some embodiments, the cell is obtained from a patient having a cancer, a microbial infection, a graft vs host disease, or an autoimmune disorder. In some embodiments, the cell is obtained from one or more healthy donors. Prior to editing by the methods and compositions disclosed herein, the cell can be obtained from a subject through a variety of non-limiting methods. T cells can be obtained from a number of non-limiting sources, for example, peripheral blood mononuclear cells, bone marrow, lymph node tissue, cord blood, thymus tissue, tissue from a site of infection, ascites, pleural effusion, spleen tissue, and tumors. Methods of collecting blood cells, isolating and enriching T cells, and expanding them ex vivo may be by methods known in the art. In some embodiments, the cell can be obtained from a cell bank, a blood bank, cell culture, or any number of T cell lines available, and known to those skilled in the art. Cells may also be obtained from a tissue biopsy, surgery, blood, plasma, serum, or other biological fluid. In some embodiments, the cell can be obtained prior to editing from one or more healthy donors, from a patient having a cancer, a microbial infection, a graft versus host infection, or an autoimmune disorder.

In some embodiments, the edited cells are expanded ex vivo prior to administering to a subject. In some embodiments, the edited cells are expanded in vivo, for example, after administering the edited cells to a subject. In some embodiments, the edited cells can be used as a medicament. In some embodiments, the medicament can be used to treat subjects having, diagnosed with, or at risk of developing a disease, disorder, or a condition, e.g., a cancer, a microbial infection such as a pathogenic infection, a bacterial infection, a viral infection, an autoimmune disorder, or a graft versus host disease. In some embodiments, the edited cells can be used in manufacture of a medicament for a treatment of a disease, disorder, or a condition, e.g., cancer, a microbial infection, such as a pathogenic infection, a bacterial infection, a viral infection, an autoimmune disorder, or a graft versus host disease. In some embodiments, the subject can be undergoing or in need of an immunosuppressive therapy.

In one aspect, provided herein are methods of treatment of a subject in need thereof. In one aspect, provided herein are methods of treatment or prevention of a disease, disorder, or condition in a subject. In some embodiments, the subject is diagnosed with, has, or is at a risk of developing a disease, disorder, or a condition. In some embodiments, the subject is in need of, or undergoing, or will be undergoing an immunotherapy, for example, an autologous immune cell immunotherapy, an allogenic immune cell immune therapy. In some embodiments, the disease, disorder, or a condition is responsive to immunotherapy, for example, an autologous immune cell immunotherapy, an allogenic immune cell immune therapy. In some embodiments, the methods disclosed herein comprise administering an effective amount of a prime editing composition disclosed herein, the edited cells disclosed herein, or a composition comprising the edited cells disclosed herein to the subject.

### Editing Efficiency

In some embodiments, editing efficiency of the prime editing compositions and method described herein can be measured by calculating the percentage of edited target genes in a population of cells introduced with the prime editing composition. In some embodiments, the editing efficiency is determined after 1 hour, 2 hours, 6 hours, 12 hours, 24 hours, 36 hours, 48 hours, 3 days, 4 days, 5 days, 7 days, 10 days, or 14 days of exposing a target gene (e.g., a TRAC gene within the genome of a cell) to a prime editing composition. In some embodiments, editing efficiency of the prime editing compositions and method described herein can be measured by calculating the percentage of edited target genes in a population of cells introduced with the prime editing composition. In some embodiments, the editing efficiency is determined after 5, 6, 7, 8, 9, 10, 11, 12, 13, 14, 15, or 16 weeks of exposing a target gene (e.g., a TRAC gene within the genome of a cell) to a prime editing composition. In some embodiments, the population of cells introduced with the prime editing composition is ex vivo. In some embodiments, the population of cells introduced with the prime editing composition is in vitro. In some embodiments, the population of cells introduced with the prime editing composition is in vivo. In some embodiments, the prime editing methods disclosed herein have an editing efficiency of at least about 1%, at least about 5%, at least about 10%, at least about 20%, at least about 30%, at least about 40%, at least about 50%, at least about 60%, at least about 70%, at least about 80%, at least about 90%, at least about 95%, or at least about 99% relative to a suitable control. In some embodiments, the prime editing methods disclosed herein have an editing efficiency of at least 25% relative to a suitable control. In some embodiments, the prime editing methods disclosed herein have an editing efficiency of at least 35% relative to a suitable control prime editing method disclosed herein has an editing efficiency of at least 30% relative to a suitable control. In some embodiments, the prime editing methods disclosed herein have an editing efficiency of at least 45% relative to a suitable control. In some embodiments, the prime editing methods disclosed herein have an editing efficiency of at least 50% relative to a suitable control. In some embodiments, editing efficiency of prime the prime editing compositions and method described herein can be measured by calculating the percentage of edited target genes in a population of cells after in vivo engraftment of the edited cells. In some embodiments, the editing efficiency is determined after 5, 6, 7, 8, 9, 10, 11, 12, 13, 14, 15, or 16 weeks of engraftment. In some embodiments, the editing efficiency is determined after 8 or 16 weeks of engraftment. In some embodiments, prime editing is able to maintain in edited cells at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or more than 95% of editing efficiency after 8 or 16 weeks post engraftment.

In some embodiments, the methods disclosed herein have an editing efficiency of at least about 1%, at least about 5%, at least about 7.5%, at least about 10%, at least about 15%, at least about 20%, at least about 30%, at least about 40%, at least about 50%, at least about 60%, at least about 70%, at least about 80%, at least about 90%, or at least about 95% of editing a primary cell relative to a suitable control.

In some embodiments, the methods disclosed herein have an editing efficiency of at least about 5%, at least about 7.5%, at least about 10%, at least about 15%, at least about 20%, at least about 30%, at least about 40%, at least about 50%, at least about 60%, at least about 70%, at least about 80%, at least about 90%, or at least about 95% of editing a target cell (e.g., a human primary cell, human iPSC, or human T cell or a progenitor or a precursor thereof) relative to a corresponding control target cell. In some embodiments, the target cell is a human cell (e.g., a human primary cell, human iPSC, or human T cell, or a progenitor or a precursor thereof).

In some embodiments, the methods disclosed herein results in integration of a donor sequence at an efficiency of at least about 5%, at least about 7.5%, at least about 10%, at least about 15%, at least about 20%, at least about 30%, at least about 40%, at least about 50%, at least about 60%, at least about 70%, at least about 80%, at least about 90%, or at least about 95% of editing a target cell or target cell population (e.g., a human primary cell, human iPSC, or human T cell or a progenitor or a precursor thereof) relative to a corresponding control target cell. In some embodiments, the target cell is a human cell (e.g., a human primary cell, human iPSC, human immune cell, or human T cell, or a progenitor or a precursor thereof).

In some embodiments, the prime editing compositions provided herein are capable of incorporating one or more intended nucleotide edits without generating a significant proportion of indels. The term “indel(s)”, as used herein, refers to the insertion or deletion of a nucleotide base within a polynucleotide, for example, a target gene. Such insertions or deletions can lead to frame shift mutations within a coding region of a gene. Indel frequency of editing can be calculated by methods known in the art. In some embodiments, indel frequency can be calculated based on sequence alignment such as the CRISPResso 2 algorithm as described in Clement et al., Nat. Biotechnol. 37 (3): 224-226 (2019), which is incorporated herein in its entirety. In some embodiments, the methods disclosed herein can have an indel frequency of less than 30%, less than 20%, less than 10%, less than 9%, less than 8%, less than 7%, less than 6%, less than 5%, less than 4%, less than 3%, less than 2%, less than 1.5%, or less than 1%. In some embodiments, any number of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a target gene (e.g., a TRAC gene within the genome of a cell) to a prime editing composition.

In some embodiments, the prime editing compositions provided herein are capable of incorporating one or more intended nucleotide edits efficiently without generating a significant proportion of indels. In some embodiments, the prime editing methods disclosed herein have an editing efficiency of at least about 1% and an indel frequency of less than 1% in a target cell, e.g., a human T-cell. In some embodiments, the prime editing methods disclosed herein have an editing efficiency of at least about 1% and an indel frequency of less than 0.5% in a target cell, e.g., a human T-cell. In some embodiments, the prime editing methods disclosed herein have an editing efficiency of at least about 1% and an indel frequency of less than 0.1% in a target cell, e.g., a human T-cell. In some embodiments, the prime editing methods disclosed herein have an editing efficiency of at least about 5% and an indel frequency of less than 1% in a target cell, e.g., a human primary cell or muscle cell. In some embodiments, the prime editing methods disclosed herein have an editing efficiency of at least about 5% and an indel frequency of less than 0.5% in a target cell, e.g., a human T-cell. In some embodiments, the prime editing methods disclosed herein have an editing efficiency of at least about 5% and an indel frequency of less than 0.1% in a target cell, e.g., a human T-cell.

In some embodiments, the prime editing methods disclosed herein have an editing efficiency of at least about 7.5% and an indel frequency of less than 1% in a target cell, e.g., a human T-cell. In some embodiments, the prime editing methods disclosed herein have an editing efficiency of at least about 7.5% and an indel frequency of less than 0.5% in a target cell, e.g., a human T-cell. In some embodiments, the prime editing methods disclosed herein have an editing efficiency of at least about 7.5% and an indel frequency of less than 0.1% in a target cell, e.g., a human T-cell.

In some embodiments, the prime editing methods disclosed herein have an editing efficiency of at least about 10% and an indel frequency of less than 1% in a target cell, e.g., a human T-cell. In some embodiments, the prime editing methods disclosed herein have an editing efficiency of at least about 10% and an indel frequency of less than 0.5% in a target cell, e.g., a human T-cell. In some embodiments, the prime editing methods disclosed herein have an editing efficiency of at least about 10% and an indel frequency of less than 0.1% in a target cell, e.g., a human T-cell.

In some embodiments, the prime editing methods disclosed herein have an editing efficiency of at least about 15% and an indel frequency of less than 1% in a target cell, e.g., a human T-cell. In some embodiments, the prime editing methods disclosed herein have an editing efficiency of at least about 15% and an indel frequency of less than 0.5% in a target cell, e.g., a human T-cell. In some embodiments, the prime editing methods disclosed herein have an editing efficiency of at least about 15% and an indel frequency of less than 0.1% in a target cell, e.g., a human T-cell.

In some embodiments, the prime editing methods disclosed herein have an editing efficiency of at least about 20% and an indel frequency of less than 1% in a target cell, e.g., a human T-cell. In some embodiments, the prime editing methods disclosed herein have an editing efficiency of at least about 20% and an indel frequency of less than 0.5% in a target cell, e.g., a human T-cell. In some embodiments, the prime editing methods disclosed herein have an editing efficiency of at least about 20% and an indel frequency of less than 0.1% in a target cell, e.g., a human T-cell.

In some embodiments, the prime editing methods disclosed herein have an editing efficiency of at least about 30% and an indel frequency of less than 1% in a target cell, e.g., a human T-cell. In some embodiments, the prime editing methods disclosed herein have an editing efficiency of at least about 30% and an indel frequency of less than 0.5% in a target cell, e.g., a human T-cell. In some embodiments, the prime editing methods disclosed herein have an editing efficiency of at least about 30% and an indel frequency of less than 0.1% in a target cell, e.g., a human T-cell.

In some embodiments, the prime editing methods disclosed herein have an editing efficiency of at least about 40% and an indel frequency of less than 1% in a target cell, e.g., a human T-cell. In some embodiments, the prime editing methods disclosed herein have an editing efficiency of at least about 40% and an indel frequency of less than 0.5% in a target cell, e.g., a human T-cell. In some embodiments, the prime editing methods disclosed herein have an editing efficiency of at least about 40% and an indel frequency of less than 0.1% in a target cell, e.g., a human T-cell.

In some embodiments, the prime editing methods disclosed herein have an editing efficiency of at least about 50% and an indel frequency of less than 1% in a target cell, e.g., a human T-cell. In some embodiments, the prime editing methods disclosed herein have an editing efficiency of at least about 50% and an indel frequency of less than 0.5% in a target cell, e.g., a human T-cell. In some embodiments, the prime editing methods disclosed herein have an editing efficiency of at least about 50% and an indel frequency of less than 0.1% in a target cell, e.g., a human T-cell.

In some embodiments, the prime editing methods disclosed herein have an editing efficiency of at least about 60% and an indel frequency of less than 1% in a target cell, e.g., a human T-cell. In some embodiments, the prime editing methods disclosed herein have an editing efficiency of at least about 60% and an indel frequency of less than 0.5% in a target cell, e.g., a human T-cell. In some embodiments, the prime editing methods disclosed herein have an editing efficiency of at least about 60% and an indel frequency of less than 0.1% in a target cell, e.g., a human T-cell.

In some embodiments, the prime editing methods disclosed herein have an editing efficiency of at least about 70% and an indel frequency of less than 1% in a target cell, e.g., a human T-cell. In some embodiments, the prime editing methods disclosed herein have an editing efficiency of at least about 70% and an indel frequency of less than 0.5% in a target cell, e.g., a human T-cell. In some embodiments, the prime editing methods disclosed herein have an editing efficiency of at least about 70% and an indel frequency of less than 0.1% in a target cell, e.g., a human T-cell.

In some embodiments, the prime editing methods disclosed herein have an editing efficiency of at least about 80% and an indel frequency of less than 1% in a target cell, e.g., a human T-cell. In some embodiments, the prime editing methods disclosed herein have an editing efficiency of at least about 80% and an indel frequency of less than 0.5% in a target cell, e.g., a human T-cell. In some embodiments, the prime editing methods disclosed herein have an editing efficiency of at least about 80% and an indel frequency of less than 0.1% in a target cell, e.g., a human T-cell.

In some embodiments, the prime editing methods disclosed herein have an editing efficiency of at least about 90% and an indel frequency of less than 1% in a target cell, e.g., a human T-cell. In some embodiments, the prime editing methods disclosed herein have an editing efficiency of at least about 90% and an indel frequency of less than 0.5% in a target cell, e.g., a human T-cell. In some embodiments, the prime editing methods disclosed herein have an editing efficiency of at least about 90% and an indel frequency of less than 0.1% in a target cell, e.g., a human T-cell.

In some embodiments, the prime editing methods disclosed herein have an editing efficiency of at least about 95% and an indel frequency of less than 1% in a target cell, e.g., a human T-cell. In some embodiments, the prime editing methods disclosed herein have an editing efficiency of at least about 95% and an indel frequency of less than 0.5% in a target cell, e.g., a human T-cell. In some embodiments, the prime editing methods disclosed herein have an editing efficiency of at least about 95% and an indel frequency of less than 0.1% in a target cell, e.g., a human T-cell. In some embodiments, any number of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a target gene (e.g., a TRAC gene within the genome of a cell) to a prime editing composition. In some embodiments, the editing efficiency is determined after 1 hour, 2 hours, 6 hours, 12 hours, 24 hours, 36 hours, 48 hours, 3 days, 4 days, 5 days, 7 days, 10 days, or 14 days of exposing a target gene (e.g., a TRAC gene within the genome of a cell) to a prime editing composition.

In some embodiments, the prime editing compositions provided herein are capable of incorporating one or more intended nucleotide edits efficiently without generating a significant proportion of indels. In some embodiments, the prime editing methods disclosed herein have an editing efficiency of at least about 1% and an indel frequency of less than 10% in a population of target cells. In some embodiments, the prime editing methods disclosed herein have an editing efficiency of at least about 1% and an indel frequency of less than 7.5% in a population of target cells. In some embodiments, the prime editing methods disclosed herein have an editing efficiency of at least about 1% and an indel frequency of less than 5% in a population of target cells. In some embodiments, the prime editing methods disclosed herein have an editing efficiency of at least about 1% and an indel frequency of less than 2.5% in a population of target cells. In some embodiments, the prime editing methods disclosed herein have an editing efficiency of at least about 1% and an indel frequency of less than 1% in a population of target cells. In some embodiments, the prime editing methods disclosed herein have an editing efficiency of at least about 1% and an indel frequency of less than 0.5% in a population of target cells. In some embodiments, the prime editing methods disclosed herein have an editing efficiency of at least about 1% and an indel frequency of less than 0.1% in a population of target cells. In some embodiments, the prime editing methods disclosed herein have an editing efficiency of at least about 5% and an indel frequency of less than 1% in a population of target cells. In some embodiments, the prime editing methods disclosed herein have an editing efficiency of at least about 5% and an indel frequency of less than 0.5% in a population of target cells. In some embodiments, the prime editing methods disclosed herein have an editing efficiency of at least about 5% and an indel frequency of less than 0.1% in a population of target cells.

In some embodiments, the prime editing methods disclosed herein have an editing efficiency of at least about 7.5% and an indel frequency of less than 10% in a population of target cells. In some embodiments, the prime editing methods disclosed herein have an editing efficiency of at least about 7.5% and an indel frequency of less than 7.5% in a population of target cells. In some embodiments, the prime editing methods disclosed herein have an editing efficiency of at least about 7.5% and an indel frequency of less than 5% in a population of target cells. In some embodiments, the prime editing methods disclosed herein have an editing efficiency of at least about 7.5% and an indel frequency of less than 2.5% in a population of target cells. In some embodiments, the prime editing methods disclosed herein have an editing efficiency of at least about 7.5% and an indel frequency of less than 1% in a population of target cells. In some embodiments, the prime editing methods disclosed herein have an editing efficiency of at least about 7.5% and an indel frequency of less than 0.5% in a population of target cells. In some embodiments, the prime editing methods disclosed herein have an editing efficiency of at least about 7.5% and an indel frequency of less than 0.1% in a population of target cells.

In some embodiments, the prime editing methods disclosed herein have an editing efficiency of at least about 10% and an indel frequency of less than 10% in a population of target cells. In some embodiments, the prime editing methods disclosed herein have an editing efficiency of at least about 10% and an indel frequency of less than 7.5% in a population of target cells. In some embodiments, the prime editing methods disclosed herein have an editing efficiency of at least about 10% and an indel frequency of less than 5% in a population of target cells. In some embodiments, the prime editing methods disclosed herein have an editing efficiency of at least about 10% and an indel frequency of less than 2.5% in a population of target cells. In some embodiments, the prime editing methods disclosed herein have an editing efficiency of at least about 10% and an indel frequency of less than 1% in a population of target cells. In some embodiments, the prime editing methods disclosed herein have an editing efficiency of at least about 10% and an indel frequency of less than 0.5% in a population of target cells. In some embodiments, the prime editing methods disclosed herein have an editing efficiency of at least about 10% and an indel frequency of less than 0.1% in a population of target cells.

In some embodiments, the prime editing methods disclosed herein have an editing efficiency of at least about 15% and an indel frequency of less than 10% in a population of target cells. In some embodiments, the prime editing methods disclosed herein have an editing efficiency of at least about 15% and an indel frequency of less than 7.5% in a population of target cells. In some embodiments, the prime editing methods disclosed herein have an editing efficiency of at least about 15% and an indel frequency of less than 5% in a population of target cells. In some embodiments, the prime editing methods disclosed herein have an editing efficiency of at least about 15% and an indel frequency of less than 2.5% in a population of target cells. In some embodiments, the prime editing methods disclosed herein have an editing efficiency of at least about 15% and an indel frequency of less than 1% in a population of target cells. In some embodiments, the prime editing methods disclosed herein have an editing efficiency of at least about 15% and an indel frequency of less than 0.5% in a population of target cells. In some embodiments, the prime editing methods disclosed herein have an editing efficiency of at least about 15% and an indel frequency of less than 0.1% in a population of target cells.

In some embodiments, the prime editing methods disclosed herein have an editing efficiency of at least about 15% and an indel frequency of less than 10% in a population of target cells. In some embodiments, the prime editing methods disclosed herein have an editing efficiency of at least about 15% and an indel frequency of less than 7.5% in a population of target cells. In some embodiments, the prime editing methods disclosed herein have an editing efficiency of at least about 15% and an indel frequency of less than 5% in a population of target cells. In some embodiments, the prime editing methods disclosed herein have an editing efficiency of at least about 15% and an indel frequency of less than 2.5% in a population of target cells. In some embodiments, the prime editing methods disclosed herein have an editing efficiency of at least about 20% and an indel frequency of less than 1% in a population of target cells. In some embodiments, the prime editing methods disclosed herein have an editing efficiency of at least about 20% and an indel frequency of less than 0.5% in a population of target cells. In some embodiments, the prime editing methods disclosed herein have an editing efficiency of at least about 20% and an indel frequency of less than 0.1% in a population of target cells.

In some embodiments, the prime editing methods disclosed herein have an editing efficiency of at least about 30% and an indel frequency of less than 10% in a population of target cells. In some embodiments, the prime editing methods disclosed herein have an editing efficiency of at least about 30% and an indel frequency of less than 7.5% in a population of target cells. In some embodiments, the prime editing methods disclosed herein have an editing efficiency of at least about 30% and an indel frequency of less than 5% in a population of target cells. In some embodiments, the prime editing methods disclosed herein have an editing efficiency of at least about 30% and an indel frequency of less than 2.5% in a population of target cells. In some embodiments, the prime editing methods disclosed herein have an editing efficiency of at least about 30% and an indel frequency of less than 1% in a population of target cells. In some embodiments, the prime editing methods disclosed herein have an editing efficiency of at least about 30% and an indel frequency of less than 0.5% in a population of target cells. In some embodiments, the prime editing methods disclosed herein have an editing efficiency of at least about 30% and an indel frequency of less than 0.1% in a population of target cells.

In some embodiments, the prime editing methods disclosed herein have an editing efficiency of at least about 40% and an indel frequency of less than 10% in a population of target cells. In some embodiments, the prime editing methods disclosed herein have an editing efficiency of at least about 40% and an indel frequency of less than 7.5% in a population of target cells. In some embodiments, the prime editing methods disclosed herein have an editing efficiency of at least about 40% and an indel frequency of less than 5% in a population of target cells. In some embodiments, the prime editing methods disclosed herein have an editing efficiency of at least about 40% and an indel frequency of less than 2.5% in a population of target cells. In some embodiments, the prime editing methods disclosed herein have an editing efficiency of at least about 40% and an indel frequency of less than 1% in a population of target cells. In some embodiments, the prime editing methods disclosed herein have an editing efficiency of at least about 40% and an indel frequency of less than 0.5% in a population of target cells. In some embodiments, the prime editing methods disclosed herein have an editing efficiency of at least about 40% and an indel frequency of less than 0.1% in a population of target cells.

In some embodiments, the prime editing methods disclosed herein have an editing efficiency of at least about 50% and an indel frequency of less than 10% in a population of target cells. In some embodiments, the prime editing methods disclosed herein have an editing efficiency of at least about 50% and an indel frequency of less than 7.5% in a population of target cells. In some embodiments, the prime editing methods disclosed herein have an editing efficiency of at least about 50% and an indel frequency of less than 5% in a population of target cells. In some embodiments, the prime editing methods disclosed herein have an editing efficiency of at least about 50% and an indel frequency of less than 2.5% in a population of target cells. In some embodiments, the prime editing methods disclosed herein have an editing efficiency of at least about 50% and an indel frequency of less than 1% in a population of target cells. In some embodiments, the prime editing methods disclosed herein have an editing efficiency of at least about 50% and an indel frequency of less than 0.5% in a population of target cells. In some embodiments, the prime editing methods disclosed herein have an editing efficiency of at least about 50% and an indel frequency of less than 0.1% in a population of target cells.

In some embodiments, the prime editing methods disclosed herein have an editing efficiency of at least about 60% and an indel frequency of less than 10% in a population of target cells. In some embodiments, the prime editing methods disclosed herein have an editing efficiency of at least about 60% and an indel frequency of less than 7.5% in a population of target cells. In some embodiments, the prime editing methods disclosed herein have an editing efficiency of at least about 60% and an indel frequency of less than 5% in a population of target cells. In some embodiments, the prime editing methods disclosed herein have an editing efficiency of at least about 60% and an indel frequency of less than 2.5% in a population of target cells. In some embodiments, the prime editing methods disclosed herein have an editing efficiency of at least about 60% and an indel frequency of less than 1% in a population of target cells. In some embodiments, the prime editing methods disclosed herein have an editing efficiency of at least about 60% and an indel frequency of less than 0.5% in a population of target cells. In some embodiments, the prime editing methods disclosed herein have an editing efficiency of at least about 60% and an indel frequency of less than 0.1% in a population of target cells.

In some embodiments, the prime editing methods disclosed herein have an editing efficiency of at least about 70% and an indel frequency of less than 10% in a population of target cells. In some embodiments, the prime editing methods disclosed herein have an editing efficiency of at least about 70% and an indel frequency of less than 7.5% in a population of target cells. In some embodiments, the prime editing methods disclosed herein have an editing efficiency of at least about 70% and an indel frequency of less than 5% in a population of target cells. In some embodiments, the prime editing methods disclosed herein have an editing efficiency of at least about 70% and an indel frequency of less than 2.5% in a population of target cells. In some embodiments, the prime editing methods disclosed herein have an editing efficiency of at least about 70% and an indel frequency of less than 1% in a population of target cells. In some embodiments, the prime editing methods disclosed herein have an editing efficiency of at least about 70% and an indel frequency of less than 0.5% in a population of target cells. In some embodiments, the prime editing methods disclosed herein have an editing efficiency of at least about 70% and an indel frequency of less than 0.1% in a population of target cells.

In some embodiments, the prime editing methods disclosed herein have an editing efficiency of at least about 80% and an indel frequency of less than 10% in a population of target cells. In some embodiments, the prime editing methods disclosed herein have an editing efficiency of at least about 80% and an indel frequency of less than 7.5% in a population of target cells. In some embodiments, the prime editing methods disclosed herein have an editing efficiency of at least about 80% and an indel frequency of less than 5% in a population of target cells. In some embodiments, the prime editing methods disclosed herein have an editing efficiency of at least about 80% and an indel frequency of less than 2.5% in a population of target cells. In some embodiments, the prime editing methods disclosed herein have an editing efficiency of at least about 80% and an indel frequency of less than 1% in a population of target cells. In some embodiments, the prime editing methods disclosed herein have an editing efficiency of at least about 80% and an indel frequency of less than 0.5% in a population of target cells. In some embodiments, the prime editing methods disclosed herein have an editing efficiency of at least about 80% and an indel frequency of less than 0.1% in a population of target cells.

In some embodiments, the prime editing methods disclosed herein have an editing efficiency of at least about 90% and an indel frequency of less than 10% in a population of target cells. In some embodiments, the prime editing methods disclosed herein have an editing efficiency of at least about 90% and an indel frequency of less than 7.5% in a population of target cells. In some embodiments, the prime editing methods disclosed herein have an editing efficiency of at least about 90% and an indel frequency of less than 5% in a population of target cells. In some embodiments, the prime editing methods disclosed herein have an editing efficiency of at least about 90% and an indel frequency of less than 2.5% in a population of target cells. In some embodiments, the prime editing methods disclosed herein have an editing efficiency of at least about 90% and an indel frequency of less than 1% in a population of target cells. In some embodiments, the prime editing methods disclosed herein have an editing efficiency of at least about 90% and an indel frequency of less than 0.5% in a population of target cells. In some embodiments, the prime editing methods disclosed herein have an editing efficiency of at least about 90% and an indel frequency of less than 0.1% in a population of target cells.

In some embodiments, the prime editing methods disclosed herein have an editing efficiency of at least about 95% and an indel frequency of less than 10% in a population of target cells. In some embodiments, the prime editing methods disclosed herein have an editing efficiency of at least about 95% and an indel frequency of less than 7.5% in a population of target cells. In some embodiments, the prime editing methods disclosed herein have an editing efficiency of at least about 95% and an indel frequency of less than 5% in a population of target cells. In some embodiments, the prime editing methods disclosed herein have an editing efficiency of at least about 95% and an indel frequency of less than 2.5% in a population of target cells. In some embodiments, the prime editing methods disclosed herein have an editing efficiency of at least about 95% and an indel frequency of less than 1% as measured in a population of target cells. In some embodiments, the prime editing methods disclosed herein have an editing efficiency of at least about 95% and an indel frequency of less than 0.5% as measured in a population of target cells. In some embodiments, the prime editing methods disclosed herein have an editing efficiency of at least about 95% and an indel frequency of less than 0.1% as measured in a population of target cells. In some embodiments, the prime editing methods disclosed herein have an editing efficiency of at least about 90% and an indel frequency of less than 10% in a population of target cells. In some embodiments, the prime editing methods disclosed herein have an editing efficiency of at least about 90% and an indel frequency of less than 7.5% in a population of target cells. In some embodiments, the prime editing methods disclosed herein have an editing efficiency of at least about 90% and an indel frequency of less than 5% in a population of target cells. In some embodiments, the prime editing methods disclosed herein have an editing efficiency of at least about 90% and an indel frequency of less than 2.5% in a population of target cells. In some embodiments, the prime editing methods disclosed herein have an editing efficiency of at least about 90% and an indel frequency of less than 1% in a population of target cells. In some embodiments, the prime editing methods disclosed herein have an editing efficiency of at least about 90%. In some embodiments, the population of target cell comprises a population of human T-cells.

In some embodiments, the prime editing composition described herein results in less than 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% off-target editing in a chromosome that includes the target gene. In some embodiments, off-target editing is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a target gene (e.g., a nucleic acid within the genome of a cell) to a prime editing composition.

In some embodiments, the prime editing methods described herein result in less than 10%, less than 9%, less than 8%, less than 7%, less than 6%, less than 5%, less than 4%, less than 3%, less than 2%, less than 1%, or less than 0.5% large deletion in edited cells. In some embodiments, the prime editing methods described herein result in less than 4% large deletion in edited cells. In some embodiments, the prime editing methods described herein result in less than 3% large deletion in edited cells. In some embodiments, the prime editing methods described herein result in less than 2% large deletion in edited cells. In some embodiments, the prime editing methods described herein result in less than 1% large deletion in edited cells. In some embodiments, the prime editing methods described herein does not result in detectable level of large deletion in edited cells.

In some embodiments, incorporation of the one or more intended nucleotide edits in the target TRAC gene decreases expression and/or function of a TRAC protein. In some embodiments, expression and/or function of a TRAC protein may be measured when expressed in a target cell. In some embodiments, incorporation of the one or more intended nucleotide edits in the target TRAC gene leads to a fold change in a level of TRAC gene expression, TRAC protein expression, TRAC protein function, or a combination thereof. In some embodiments, a change (e.g., a decrease) in the level of TRAC gene expression, TRAC protein expression, TRAC protein function, or a combination thereof can comprise a fold change of, e.g., 2-fold, 3-fold, 4-fold, 5-fold, 6-fold, 7-fold, 8-fold, 9-fold, 10-fold, 15-fold, 20-fold, 25-fold, 30-fold, 40-fold, 50-fold, 60-fold, 70-fold, 80-fold, 90-fold, 100-fold or more as compared to expression in a suitable control cell. In some embodiments, a change (e.g., a decrease) in the level of a TRAC protein function can comprise a fold change of, e.g., 2-fold, 3-fold, 4-fold, 5-fold, 6-fold, 7-fold, 8-fold, 9-fold, 10-fold, 15-fold, 20-fold, 25-fold, 30-fold, 40-fold, 50-fold, 60-fold, 70-fold, 80-fold, 90-fold, 100-fold or more as compared to expression in a suitable control cell. In some embodiments, incorporation of the one or more intended nucleotide edits in the target TRAC gene decreases expression of a TRAC gene, TRAC mRNA and/or TRAC protein e.g., by at least 1%, 2%, 5%, 10%, 15%, 20%, 25%, 30%, 35%, 40%, 45%, 50%, 55%, 60%, 65%, 70%, 75%, 80%, 85%, 90%, 95%, 99% or more as compared to the expression in a suitable control cell. In some embodiments, incorporation of the one or more intended nucleotide edits in the target TRAC gene results in abolished or complete loss of expression of a TRAC gene, and/or a TRAC protein as compared to the expression in a suitable control cell. As used herein “abolished expression” or “complete loss of expression” means that the expression of TRAC protein is substantially not detected.

In some embodiments, incorporation of the one or more intended nucleotide edits in the target TRAC gene decreases function of TRAC protein e.g., by at least 10%, 20%, 30%, 40%, 50%, 60%, 70%, 80%, 90%, 95%, 99% or more as compared to that in a suitable control cell. In some embodiments, incorporation of the one or more intended nucleotide edits in the target TRAC gene results in abolished or complete loss of function of the TRAC protein as compared to that in a suitable control cell. As used herein “abolished function” or “complete loss of function” means that the function of TRAC protein is substantially not detected. In some embodiments, the function of a TRAC protein comprises associating with a T cell receptor B chain, associating with one or more CD3 δ, γ, ε, and ζ chains and/or expression of a TCR (e.g., a functional TCR) on the surface of a cell.

In some embodiments, incorporation of the one or more intended nucleotide edits in the target TRAC gene increases expression of a truncated TRAC protein e.g., by at least 10%, 20%, 30%, 40%, 50%, 60%, 70%, 80%, 90%, 95%, 99% or more as compared to expression of a full length wild type TRAC protein.

In some embodiments, a suitable control cell is a cell that comprises a wild type TRAC gene, or is a cell not introduced or contacted with a prime editing composition described herein. In some embodiments, a suitable control cell is a cell from a healthy subject.

In some embodiments, components of a prime editing composition described herein are provided to a target cell in vitro. In some embodiments, components of a prime editing composition described herein are provided to a target cell ex vivo. In some embodiments, components of a prime editing composition described herein are provided to a target cell in vivo.

In some embodiments, incorporation of the one or more intended nucleotide edits in the target TRAC gene that reduces or abolishes expression and function of the protein encoded by the endogenous TRAC gene.

In some embodiments, a decrease in expression and/or function of proteins of the TRAC gene can be measured by a functional assay. In some embodiments, expression and/or function of a exogenous protein encoded by a donor sequence introduced by prime editing and recombination can be measured by a functional assay.

Functional assays can include ex vivo and/or in vivo assays.

In some embodiments, a protein expression change can be measured by a protein expression assay. In some embodiments, the TRAC protein expression can be measured using antibody testing. In some embodiments, an antibody can comprise anti-TRAC. In some embodiments, the TRAC protein expression can be measured using ELISA, mass spectrometry, Western blot, sodium dodecyl sulfate polyacrylamide gel electrophoresis (SDS-PAGE), high performance liquid chromatography (HPLC), electrophoresis, or any combination thereof. In some embodiments, a protein assay can comprise SDS-PAGE and densitometric analysis of a Coomassie Blue-stained gel.

In some embodiments, the method provided herein comprises administering to a subject an effective amount of a prime editing composition or a pharmaceutical composition comprising a prime editing composition described herein (i.e., for in vivo T cell editing). For example, in some embodiments, the method comprises administering to a subject an effective amount of an LNP comprising a prime editing composition described herein. In some embodiments, the method provided herein comprises administering to a subject an effective amount of a pharmaceutical composition comprising a prime edited cell or a population of prime edited cells described herein. In some embodiments, the method comprises directly administering prime editing compositions provided herein to a subject, e.g., using a LNP that targets T cells and comprises a prime editing composition, e.g., a pair of dual prime editing PEgRNAs, an mRNA encoding a prime editor, and optionally (i) an mRNA encoding a recombinase that recognizes a RSS inserted by the PEgRNAs, and (ii) a DNA polynucleotide comprising a donor sequence and one or more RSSs recognized by the recombinase. The pharmaceutical compositions can be administered to, e.g., a human subject in need of immune therapy, for example a human subject suffering from, having, susceptibility to, or at risk for a disease or condition, e.g., a cancer, a microbial infection, an autoimmune disorder, or a graft vs host disease. Identifying a subject in need of such treatment can be in the judgment of a subject or a health care professional and can be subjective (e.g., opinion) or objective (e.g., measurable by a test or diagnostic method).

In some embodiments, the method comprises directly administering prime editing compositions provided herein to a subject. The prime editing compositions described herein can be delivered, e.g., as LNPs. The prime editing compositions can be formulated with any pharmaceutically acceptable carrier described herein or known in the art for administering directly to a subject. Components of a prime editing composition or a pharmaceutical composition thereof may be administered to the subject, contacted to a TRAC gene, or introduced in a cell comprising a TRAC gene simultaneously or sequentially. For example, in some embodiments, the method comprises administering, contacting with, or introducing a prime editing composition, or pharmaceutical composition thereof, comprising prime editor complexes that comprises (i) a prime editor fusion protein and a first PERNA and (ii) a prime editor fusion protein and a second PEgRNA to a subject. In some embodiments, the method comprises administering, contacting with, or introducing a polynucleotide or vector encoding a prime editor to a subject simultaneously with the two PEgRNAs. In some embodiments, the method comprises administering, contacting with, or introducing a polynucleotide or vector encoding a prime editor to a subject before administration of the two PEgRNAs. In some embodiments, the two PEgRNAs are administered simultaneously. In some embodiments, the two PEgRNAs are administered sequentially. In some embodiments, a first PEgRNA is administered with a prime editor and a second PEgRNA is administered after administration of the first PEgRNA and prime editor. In some embodiments, a first PEgRNA is administered with a prime editor and a second PEgRNA is administered before administration of the first PEgRNA and prime editor.

Suitable routes of administrating the prime editing compositions to a subject include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration. In some embodiments, the compositions described are administered intraperitoneally, intravenously, or by direct injection or direct infusion. In some embodiments, the compositions described are administered by direct injection into the muscle of a subject. In some embodiments, the compositions described herein are administered to a subject by injection, by means of a catheter, by means of a suppository, or by means of an implant.

In some embodiments, the method comprises to a subject an effective amount of the edited cells (e.g., a population of edited cells) or a composition comprising the edited cells (e.g., a population of edited cells) described herein (e.g., edited cells that are generated by the methods of the disclosure. In some embodiments, the edited cells are allogeneic. In some embodiments, allogeneic cells are or have been contacted ex vivo with a prime editing composition or pharmaceutical composition thereof and are introduced into a subject (e.g., a human subject) in need thereof. In some embodiments, the edited cells are autologous to the subject. In some embodiments, cells are removed from a subject and contacted ex vivo with a prime editing composition or pharmaceutical composition thereof and are re-introduced into the subject.

In some embodiments, cells are contacted ex vivo with one or more components of a prime editing composition. In some embodiments, the ex vivo-contacted cells are introduced into the subject, and the subject is administered in vivo with one or more components of a prime editing composition. For example, in some embodiments, cells are contacted ex vivo with a prime editor and introduced into a subject. In some embodiments, the subject is a human subject. In some embodiments, the subject is then administered with the PEgRNAs, or polynucleotides encoding the PEgRNAs. In some embodiments, the ex vivo contacted cells are allogenic. In some embodiments, the ex vivo contacted cells are autologous. In some embodiments, cells contacted with the prime editing composition are determined for incorporation of the one or more intended nucleotide edits in the genome before re-introduction into the subject. In some embodiments, the cells are enriched for incorporation of the one or more intended nucleotide edits in the genome before re-introduction into the subject.

In some embodiments, the cells of the disclosure are obtained from a subject in need of an immune cell immunotherapy (e.g., a CAR-T cell therapy) prior to ex vivo editing. In some embodiments, the autologous cells obtained from a subject prior to editing are cultured prior to editing. In some embodiments, the autologous cells obtained from a subject for ex vivo prime editing, are cultured, and edited shortly after they are obtained. In some embodiments, the autologous cells obtained from a subject and stored for future use (e.g., future prime editing).

In some embodiments, the edited cells are allogenic to the subject.

The prime editing composition or components thereof may be introduced into a cell by any delivery approaches as described herein, including LNP administration, RNP administration, electroporation, nucleofection, transfection, viral transduction, microinjection, cell membrane disruption and diffusion, or any other approach known in the art.

The cells edited with prime editing can be introduced into the subject by any route known in the art. In some embodiments, the edited cells are administered to a subject by direct infusion. In some embodiments, the edited cells are administered to a subject by intravenous infusion. In some embodiments, the edited cells are administered to a subject as implants.

The pharmaceutical compositions, prime editing compositions, and cells, as described herein, can be administered in effective amounts. In some embodiments, the effective amount depends upon the mode of administration. In some embodiments, the effective amount depends upon the stage of the condition, the age and physical condition of the subject, the nature of concurrent therapy, if any, and like factors well-known to the medical practitioner.

The specific dose administered can be a uniform dose for each subject. Alternatively, a subject's dose can be tailored to the approximate body weight of the subject. Other factors in determining the appropriate dosage can include the disease or condition to be treated or prevented, the severity of the disease, the route of administration, and the age, sex and medical condition of the patient.

In embodiments wherein components of a prime editing composition are administered sequentially, the time between sequential administration can be at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days.

In some embodiments, a method of monitoring treatment progress is provided. In some embodiments, the method includes the step of determining a level of diagnostic marker, for example, expression of T-cell receptor, expression of a transgene, or measurement associated with a change in T-cell function and/or activity, in a subject who has been administered an effective amount of a prime editing composition or prime edited cells described herein. The level of the diagnostic marker determined in the method can be compared to known levels of the marker in either healthy normal controls or in other afflicted subjects to establish the subject's disease status.

Various aspects of this disclosure provide kits comprising a prime editing composition. In one embodiment, a kit comprises a prime editing composition comprising a pair of PEgRNAs (i.e., a first PEgRNA and a second PEgRNA) and a prime editor. In one embodiment, a kit comprises a prime editing composition comprising a pair of PEgRNAs (i.e., a first PEgRNA and a second PEgRNA) and a prime editor fusion protein. In some embodiments, the kit comprises a pair of PEgRNAs (i.e., a first PEgRNA and a second PEgRNA) and a polynucleotide encoding a prime editor. In some embodiments, the kit comprises a pair of PEgRNAs (i.e., a first PEgRNA and a second PEgRNA) and a polynucleotide encoding a prime editor fusion protein. In some embodiments, the kit further provides components for delivery of the PEgRNAs and/or the prime editor. In some embodiments, the kit further provides components for delivery of the PEgRNAs and/or the prime editor fusion protein. In some embodiments, the kit further provides components for delivery of the PEgRNAs and/or the polynucleotide encoding the prime editor. In some embodiments, the kit further provides components for delivery of the PEgRNAs and/or the polynucleotide encoding the prime editor fusion protein.

In some embodiments, the kit provides instructions for using the components of the kit for prime editing. The instructions will generally include information about the use of the kit for editing nucleic acid molecules. In other embodiments, the instructions include at least one of the following: precautions; warnings; clinical studies; and/or references. The instructions may be printed directly on the container (when present), or as a label applied to the container, or as a separate sheet, pamphlet, card, or folder supplied in or with the container. In a further embodiment, a kit can comprise instructions in the form of a label or separate insert (package insert) for suitable operational parameters. In yet another embodiment, the kit can comprise one or more containers with appropriate positive and negative controls or control samples, to be used as standard(s) for detection, calibration, or normalization. The kit can further comprise a second container comprising a pharmaceutically acceptable buffer, such as (sterile) phosphate-buffered saline, Ringer's solution, or dextrose solution. It can further include other materials desirable from a commercial and user standpoint, including other buffers, diluents, filters, needles, syringes, and package inserts with instructions for use.

### Delivery

Prime editing compositions described herein can be delivered to a cellular environment with any approach known in the art. Components of a prime editing composition can be delivered to a cell by the same mode or different modes. For example, in some embodiments, a prime editor can be delivered as a polypeptide or a polynucleotide (DNA or RNA) encoding the polypeptide. In some embodiments, a PEgRNA can be delivered directly as an RNA or as a DNA encoding the PEgRNA.

In some embodiments, a prime editing composition component is encoded by a polynucleotide, a vector, or a construct. In some embodiments, a prime editor polypeptide and PEgRNAs is encoded by a polynucleotide. In some embodiments, the polynucleotide encodes a prime editor fusion protein comprising a DNA binding domain and a DNA polymerase domain. In some embodiments, the polynucleotide encodes a DNA polymerase domain of a prime editor. In some embodiments, the polynucleotide encodes a DNA binding domain of a prime editor. In some embodiments, the polynucleotide encodes a portion of a prime editor protein, for example, a N-terminal portion of a prime editor fusion protein connected to an intein-N. In some embodiments, the polynucleotide encodes a portion of a prime editor protein, for example, a C-terminal portion of a prime editor fusion protein connected to an intein-C. In some embodiments, the polynucleotide encodes a PERNA. In some embodiments, the polypeptide encodes two or more components of a prime editing composition, for example, a prime editor fusion protein and a PEgRNA.

In some embodiments, the polynucleotide encoding one or more prime editing composition components that is delivered to a target cell is integrated into the genome of the cell for long-term expression, for example, by a retroviral vector. In some embodiments, the polynucleotide delivered to a target cell is expressed transiently. For example, the polynucleotide may be delivered in the form of mRNA, or a non-integrating vector (non-integrating virus, plasmids, minicircle DNAs) for episomal expression.

In some embodiments, a polynucleotide encoding one or more prime editing system components can be operably linked to a regulatory element, e.g., a transcriptional control element, such as a promoter. In some embodiments, the polynucleotide is operably linked to multiple control elements. Depending on the expression system utilized, any of a number of suitable transcription and translation control elements, including constitutive and inducible promoters, transcription enhancer elements, transcription terminators, etc. may be used in the expression vector (e.g., U6 promoter or H1 promoter).

In some embodiments, the polynucleotide encoding one or more prime editing composition components is a part of, or is encoded by, a vector. In some embodiments, the vector is a viral vector. In some embodiments, the vector is a non-viral vector.

Non-viral vector delivery systems can include DNA plasmids, RNA (e.g., a transcript of a vector described herein), naked nucleic acid, and nucleic acid complexed with a delivery vehicle, such as a liposome. In some embodiments, the polynucleotide is provided as an RNA, e.g., a mRNA or a transcript. Any RNA of the prime editing systems, for example a guide RNA or a base editor-encoding mRNA, can be delivered in the form of RNA. In some embodiments, one or more components of the prime editing system that are RNAs is produced by direct chemical synthesis or may be transcribed in vitro from a DNA. In some embodiments, mRNA that encodes a prime editor polypeptide is generated using in vitro transcription. Guide polynucleotides (e.g., PEgRNA) can also be transcribed using in vitro transcription from a cassette containing a T7 promoter, followed by the sequence “GG”, and guide polynucleotide sequence. In some embodiments, the prime editor encoding mRNA and/or PERNA(s) are synthesized in vitro using an RNA polymerase enzyme (e.g., T7 polymerase, T3 polymerase, SP6 polymerase, etc.). Once synthesized, the RNA can directly contact a target TRAC gene or can be introduced into a cell using any suitable technique for introducing nucleic acids into cells (e.g., microinjection, electroporation, or transfection). In some embodiments, the prime editor-coding sequences and/or the PEgRNAs are modified to include one or more modified nucleoside e.g., using pseudo-U or 5-Methyl-C.

Methods of non-viral delivery of nucleic acids can include lipofection, electroporation, nucleofection, microinjection, biolistics, virosomes, liposomes, immunoliposomes, polycation or lipid: nucleic acid conjugates, nanoparticles, cell penetrating peptides and associated conjugated molecules and chemistry, naked DNA, artificial virions, cell membrane disruption by a microfluidics device, and agent-enhanced uptake of DNA. Cationic and neutral lipids that are suitable for efficient receptor-recognition lipofection of polynucleotides can be used. Delivery can be to cells (e.g., in vitro or ex vivo administration) or target tissues (e.g., in vivo administration). The preparation of lipid: nucleic acid complexes, including targeted liposomes such as immunolipid complexes, can be used.

Viral vector delivery systems can include DNA and RNA viruses, which can have either episomal or integrated genomes after delivery to the cell. RNA or DNA viral based systems can be used to target specific cells and trafficking the viral payload to an organelle of the cell. Viral vectors can be administered directly (in vivo) or they can be used to treat cells in vitro, and the modified cells can optionally be administered after delivery (ex vivo).

In some embodiments, the viral vector is a retroviral, lentiviral, adenoviral, adeno-associated viral or herpes simplex viral vector. Retroviral vectors can include those based upon murine leukemia virus (MuLV), gibbon ape leukemia virus (GaLV), simian immunodeficiency virus (SIV), human immunodeficiency virus (HIV), and combinations thereof. In some embodiments, the retroviral vector is a lentiviral vector. In some embodiments, the retroviral vector is a gamma retroviral vector. In some embodiments, the viral vector is an adenoviral vector. In some embodiments, the viral vector is an adeno-associated virus (AAV) vector.

In some embodiments, polynucleotides encoding one or more prime editing composition components are packaged in a virus particle. Packaging cells can be used to form virus particles that can infect a target cell. Such cells can include 293 cells, (e.g., for packaging adenovirus), and ψ2 cells or PA317 cells (e.g., for packaging retrovirus). Viral vectors can be generated by producing a cell line that packages a nucleic acid vector into a viral particle. The vectors can contain the minimal viral sequences required for packaging and subsequent integration into a host. The vectors can contain other viral sequences being replaced by an expression cassette for the polynucleotide(s) to be expressed. The missing viral functions can be supplied in trans by the packaging cell line. For example, AAV vectors can comprise ITR sequences from the AAV genome which are required for packaging and integration into the host genome. In some embodiment, the polynucleotides are a DNA polynucleotide. In some embodiment, the polynucleotides are an RNA polynucleotide; e.g., an mRNA polynucleotide.

In some embodiments, the AAV vector is selected for tropism to a particular cell, tissue, organism. In some embodiments, the AAV vector is pseudotyped, e.g., AAV5/8. In some embodiments, polynucleotides encoding one or more prime editing composition components are packaged in a first AAV and a second AAV. In some embodiments, the polynucleotides encoding one or more prime editing composition components are packaged in a first rAAV and a second rAAV.

In some embodiments, dual AAV vectors are generated by splitting a large transgene expression cassette in two separate halves (5′ and 3′ ends that encode N-terminal portion and C-terminal portion of, e.g., a prime editor polypeptide), where each half of the cassette is no more than 5 kb in length, optionally no more than 4.7 kb in length, and is packaged in a single AAV vector. In some embodiments, the full-length transgene expression cassette is reassembled upon co-infection of the same cell by both dual AAV vectors. In some embodiments, a portion or fragment of a prime editor polypeptide, e.g., a Cas9 nickase, is fused to an intein. The portion or fragment of the polypeptide can be fused to the N-terminus or the C-terminus of the intein. In some embodiments, an N-terminal portion of the polypeptide is fused to an intein-N, and a C-terminal portion of the polypeptide is separately fused to an intein-C. In some embodiments, a portion or fragment of a prime editor fusion protein is fused to an intein and fused to an AAV capsid protein. In some embodiments, intein-N may be fused to the N-terminal portion of a first domain described herein, and intein-C may be fused to the C-terminal portion of a second domain described herein for the joining of the N-terminal portion to the C-terminal portion, thereby joining the first and second domains. In some embodiments, the first and second domains are each independently chosen from a DNA binding domain or a DNA polymerase domain. The intein, nuclease and capsid protein can be fused together in any arrangement (e.g., nuclease-intein-capsid, intein-nuclease-capsid, capsid-intein-nuclease, etc.). In some embodiments, a polynucleotide encoding a prime editor fusion protein is split in two separate halves, each encoding a portion of the prime editor fusion protein and separately fused to an intein. In some embodiments, each of the two halves of the polynucleotide is packaged in an individual AAV vector of a dual AAV vector system. In some embodiments, each of the two halves of the polynucleotide is no more than 5 kb in length, optionally no more than 4.7 kb in length. In some embodiments, the full-length prime editor fusion protein is reassembled upon co-infection of the same cell by both dual AAV vectors expression of both halves of the prime editor fusion protein, and self-excision of the inteins. In some embodiments, the in vivo use of dual AAV vectors results in the expression of full-length prime editor fusion proteins. In some embodiments, the use of the dual AAV vector platform allows viable delivery of transgenes of greater than about 4.5, 4.6, 4.7, 4.8, 4.9, or 5.0 kb in size.

In some embodiments, an intein is inserted at a splice site within a Cas protein. In some embodiments, insertion of an intein disrupts a Cas activity. As used herein, “intein” refers to a self-splicing protein intron (e.g., peptide), e.g., which ligates flanking N-terminal and C-terminal exteins (e.g., fragments to be joined). In some embodiments, an intein may comprise a polypeptide that is able to excise itself and join exteins with a peptide bond (e.g., protein splicing). In some embodiments, an intein of a precursor gene comes from two genes (e.g., split intein). In some embodiments, an intein may be a synthetic intein. Non-limiting examples of intein pairs that may be used in accordance with the present disclosure include: dnaE-n and dnaE-c. a 4-hydroxytamoxifen (4-HT)-responsive intein, an iCas molecule, a Ssp DnaX intein, Ter DnaE3 intein, Ter ThyX intein, Rma DnaB intein, Cfa DnaE intein, Ssp GyrB intein, and Rma DnaB intein. In some embodiments, intein fragments may be fused to the N terminal and C-terminal portion of a split Cas protein respectively for joining the fragments of split Cas9.

In some embodiments, the split Cas9 system may be used in general to bypass the packing limit of the viral delivery vehicles. In some embodiments, a split Cas9 may be a Type II CRISPR system Cas9. In some embodiments, a first nucleic acid encodes a first portion of the Cas9 protein having a first split-intein and wherein the second nucleic acid encodes a second portion of the Cas9 protein having a second split-intein complementary to the first split-intein and wherein the first portion of the Cas9 protein and the second portion of the Cas9 protein are joined together to form the Cas9 protein. In some embodiments, the first portion of the Cas9 protein is the N-terminal fragment of the Cas9 protein and the second portion of the Cas9 protein is the C-terminal fragment of the Cas9 protein. In some embodiments, a split site may be selected which are surface exposed due to the sterical need for protein splicing.

In some embodiments, a Cas protein may be split into two fragments at any C, T, A, or S. In some embodiments, a Cas9 may be intein split at residues 203-204, 280-292, 292-364, 311-325, 417-438, 445-483, 468-469, 481-502, 513-520, 522-530, 565-637, 696-707, 713-714, 795-804, 803-810, 878-887, and 1153-1154. In some embodiments, protein is divided into two fragments at SpCas9 T310, T313, A456, S469, or C574. In some embodiments, split Cas9 fragments across different split pairs yield combinations that provided the complete polypeptide sequence activate gene expression even when fragments are partially redundant. In some embodiments, a functional Cas9 protein may be reconstituted from two inactive split-Cas9 peptides in the presence of gRNA by using a split-intein protein splicing strategy. In some embodiment, the split Cas9 fragments are fused to either a N-terminal intein fragment or a C-terminal intein fragment, which can associate with each other and catalytically splice the two split Cas9 fragments into a functional reconstituted Cas9 protein. In some embodiments, a split-Cas9 can be packaged into self-complementary AAV. In some embodiments, a split-Cas9 comprises a 2.5 kb and a 2.2 kb fragment of S. pyogenes Cas9 coding sequences.

In some embodiments, a split-Cas9 architecture reduces the length and/or size of the coding sequences of a viral vector, e.g., AAV.

A target cell can be transiently or non-transiently transfected with one or more vectors described herein. A cell can be transfected as it naturally occurs in a subject. A cell can be taken or derived from a subject and transfected. A cell can be derived from cells taken from a subject, such as a cell line. In some embodiments, a cell transfected with one or more vectors described herein can be used to establish a new cell line comprising one or more vector-derived sequences. In some embodiments, a cell transiently transfected with the compositions of the disclosure (such as by transient transfection of one or more vectors, or transfection with RNA), and modified through the activity of a prime editor, can be used to establish a new cell line comprising cells containing the modification but lacking any other exogenous sequence. Any suitable vector compatible with the host cell can be used with the methods of the disclosure. Non-limiting examples of vectors include pXT1, pSG5, pSVK3, pBPV, pMSG, and pSVLSV40.

In some embodiments, a prime editor protein can be provided to cells as a polypeptide. In some embodiments, the prime editor protein is fused to a polypeptide domain that increases solubility of the protein. In some embodiments, the prime editor protein is formulated to improve solubility of the protein.

In some embodiment, a prime editor polypeptide is fused to a polypeptide permeant domain to promote uptake by the cell. In some embodiments, the permeant domain is a peptide, a peptidomimetic, or a non-peptide carrier. For example, a permeant peptide may be derived from the third alpha helix of Drosophila melanogaster transcription factor Antennapaedia, referred to as penetratin, which comprises the amino acid sequence RQIKIWFQNRRMKWKK (SEQ ID NO: 1248). As another example, the permeant peptide can comprise the HIV-1 tat basic region amino acid sequence, which may include, for example, amino acids 49-57 of naturally-occurring tat protein. Other permeant domains can include poly-arginine motifs, for example, the region of amino acids 34-56 of HIV-1 rev protein, nona-arginine (SEQ ID NO: 1249), and octa-arginine (SEQ ID NO: 1250). The nona-arginine (R9) sequence (SEQ ID NO: 1249) can be used. The site at which the fusion can be made may be selected in order to optimize the biological activity, secretion or binding characteristics of the polypeptide.

In some embodiments, a prime editor polypeptide is produced in vitro or by host cells, and it may be further processed by unfolding, e.g., heat denaturation, DTT reduction, etc. and may be further refolded. In some embodiments, a prime editor polypeptide is prepared by in vitro synthesis. Various commercial synthetic apparatuses can be used. By using synthesizers, naturally occurring amino acids can be substituted with unnatural amino acids. In some embodiments, a prime editor polypeptide is isolated and purified in accordance with recombinant synthesis methods, for example, by expression in a host cell and the lysate purified using HPLC, exclusion chromatography, gel electrophoresis, affinity chromatography, or other purification technique.

In some embodiments, a prime editing composition, for example, prime editor polypeptide components and PERNA(s) are introduced to a target cell by nanoparticles. In some embodiments, the prime editor polypeptide components and the PEgRNA form a complex in the nanoparticle. Any suitable nanoparticle design can be used to deliver genome editing system components or nucleic acids encoding such components. In some embodiments, the nanoparticle is inorganic. In some embodiments, the nanoparticle is organic. In some embodiments, a prime editing composition is delivered to a target cell, e.g., a muscle cell, in an organic nanoparticle, e.g., a lipid nanoparticle (LNP) or polymer nanoparticle.

In some embodiments, LNPs are formulated from cationic, anionic, neutral lipids, or combinations thereof. In some embodiments, neutral lipids, such as the fusogenic phospholipid DOPE or the membrane component cholesterol, are included to enhance transfection activity and nanoparticle stability. In some embodiments, LNPs are formulated with hydrophobic lipids, hydrophilic lipids, or combinations thereof. Lipids may be formulated in a wide range of molar ratios to produce an LNP. Any lipid or combination of lipids that are known in the art can be used to produce an LNP. Exemplary lipids used to produce LNPs are provided in Table 28 below.

In some embodiments, components of a prime editing composition form a complex prior to delivery to a target cell. For example, a prime editor fusion protein and a PEgRNA can form a complex prior to delivery to the target cell. In some embodiments, a prime editing polypeptide (e.g., a prime editor fusion protein) and a guide polynucleotide (e.g., a PEgRNA) form a ribonucleoprotein (RNP) for delivery to a target cell. In some embodiments, the RNP comprises a prime editor fusion protein in complex with a PEgRNA. RNPs may be delivered to cells using known methods, such as electroporation, nucleofection, or cationic lipid-mediated methods, or any other approaches known in the art. In some embodiments, delivery of a prime editing composition or complex to the target cell does not require the delivery of foreign DNA into the cell. In some embodiments, the RNP comprising the prime editing complex is degraded over time in the target cell. Exemplary lipids for use in nanoparticle formulations and/or gene transfer are shown in Table 28 below.

TABLE 28
Exemplary lipids for nanoparticle formulation or gene transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3-phosphatidylethanolamine | DOPE | Helper |
| Cholesterol | | Helper |
| N41-(2,3-Dioleyloxy)prophyliN,N,N-trimethylammonium | DOTMA | Cationic |
| chloride | | |
| 1,2-Dioleoyloxy-3-trimethylammonium-propane | DOGS | Cationic |
| Dioctadecylamidoglycylspermine | | |
| N-(3-Aminopropyl)-N,N-dimethyl-2,3-bis(dodecyloxy)-1- | GAP-DLRIE | Cationic |
| propanaminium bromide | | |
| Cetyltrimethylammonium bromide | CTAB | Cationic |
| 6-Lauroxyhexyl omithinate | LHON | Cationic |
| 1-(2,3-Dioleoyloxypropyl)-2,4,6-trimethylpyridinium | 20c | Cationic |
| 2,3-Dioleyloxy-N-P(spenninecarboxamido-ethy1J-N,Ndimethyl- | DOSPA | Cationic |
| 1-propanatninium trifluoroacetate | | |
| 1,2-Dioley1-3-trimethylamtnonium-propane | DOPA | Cationic |
| N-(2-Hydroxyethyl)-N,N-dimethy1-2,3-bis(tetradecyloxy)-1- | MDRIE | Cationic |
| propanaminium bromide | | |
| Dimyristooxypropyl dimethyl hydroxyethyl ammonium bromide | DMRI | Cationic |
| 3β-[N-(N′,N′-Dimethylaminoethane)-carbamoyl]cholesterol | DC-Chol | Cationic |
| Bis-guanidium-tren-cholesterol | BGTC | Cationic |
| 1,3-Diodeoxy-2-(6-carboxy-spermy1)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxyethyl)]- | CLIP-1 | Cationic |
| dimethylammonium chloride | | |
| rac-[2(2,3-Dihexadecyloxypropyloxymethyloxy) | CLIP-6 | Cationic |
| ethyl]trimethylammoniun bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylpho sphocholine | DSEPC | Cationic |
| N-Palmitoyl D-erythro-sphingosyl carbamoyl-spenmine | CCS | Cationic |
| N-t-Butyl-N0-tetradecyl-3-tetradecylaminopropionamidine | diC14-amidine | Cationic |
| Octadecenolyoxy[ethyl-2-heptadecenyl-3 hydroxyethyl] | DOTIM | Cationic |
| imidazolinium chloride | | |
| N1-Cholesteryloxycarbonyl-3,7-diazanonane-1,9-diamine | CDAN | Cationic |
| 2-(3-Bis(3-amino-propy1)-amino]propylamino)- | RPR209120 | Cationic |
| Nditetradecylcarbamoylme-ethyl-acetamide | | |
| 1,2-dilinoleyloxy-3-dimethylaminopropane | DLinDMA | Cationic |
| 2,2-dilinoley1-4-dimethylaminoethyl-[1,3]-dioxolane | DLin-KC2- | Cationic |
| | DMA | |
| dilinoleyl-methyl-4-dimethylaminobutyrate | DLin-MC3- | Cationic |
| | DMA | |

Exemplary polymers for use in nanoparticle formulations and/or gene transfer are shown in Table 29 below.

TABLE 29
Exemplary polymers for nanoparticle formulation or gene transfer

| Polymer | Abbreviation |
|---|---|
| Poly(ethylene)glycol | PEG |
| Polyethylenimine | PEI |
| Dithiobis (succinimidylpropionate) | DSP |
| Dimethyl-3,3′-dithiobispropionimidate | DTBP |
| Poly(ethylene imine)biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amidoethylenimine) | SS_PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-dodacylated chitosam | |
| Histone | |
| Collagen | |
| Dextran-spermine | D-SPM |

Exemplary delivery methods for polynucleotides encoding prime editing composition components are shown in Table 30 below.

TABLE 30
Exemplary Delivery Methods

| | | Delivery | | | |
|---|---|---|---|---|---|
| | | into Non- | | | Type of |
| | | Dividing | Duration of | Genome | Molecule |
| Delivery | Vector/Mode | Cells | Expression | Integration | Delivered |
| Physical | (e.g., | YES | Transient | NO | Nucleic |
| | electroporation, | | | | Acids and |
| | particle gun, | | | | Proteins |
| | Calcium phosphate | | | | |
| | transfection) | | | | |
| Viral | Retrovirus | NO | Stable | YES | RNA |
| | Lentivirus | YES | Stable | YES/NO with | RNA |
| | | | | modification | |
| | Adenovirus | YES | Transient | NO | DNA |
| | Adeno-Associated | YES | Stable | NO | DNA |
| | Virus (AAV) | | | | |
| | Vaccinia Virus | YES | Very | NO | DNA |
| | | | Transient | | |
| | Herpes Simplex | YES | Stable | NO | DNA |
| | Virus | | | | |
| Non-Viral | Cationic | YES | Transient | Depends on | Nucleic acids |
| | | | | what is | and Proteins |
| | | | | delivered | |
| | Polymeric | YES | Transient | NO | Nucleic |
| | Nanoparticles | | | | Acids |
| Biological | Attenuated | YES | Transient | NO | Nucleic |
| | Bacteria | | | | Acids |
| Non-Viral | Engineered | YES | Transient | NO | Nucleic |
| | Bacteriophages | | | | Acids |
| Delivery | Mammalian Virus- | YES | Transient | NO | Nucleic |
| | like Particles | | | | Acids |
| Vehicles | Biological | YES | Transient | NO | Nucleic |
| | liposomes: | | | | Acids |
| | Erythrocyte Ghosts | | | | |
| | and Exosomes | | | | |

The prime editing compositions of the disclosure, whether introduced as polynucleotides or polypeptides, can be provided to the cells for about 30 minutes to about 24 hours, e.g., 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which can be repeated with a frequency of about every day to about every 4 days, e.g., every 1.5 days, every 2 days, every 3 days, or any other frequency from about every day to about every four days. The compositions may be provided to the subject cells one or more times, e.g., one time, twice, three times, or more than three times, and the cells allowed to incubate with the agent(s) for some amount of time following each contacting event e.g., 16-24 hours. In cases in which two or more different prime editing system components, e.g., two different polynucleotide constructs are provided to the cell (e.g., different components of the same prime editing system, or two different guide nucleic acids that are complementary to different sequences within the same or different target genes), the compositions may be delivered simultaneously (e.g., as two polypeptides and/or nucleic acids). Alternatively, they may be provided sequentially, e.g., one composition being provided first, followed by a second composition.

The prime editing compositions and pharmaceutical compositions of the disclosure, whether introduced as polynucleotides or polypeptides, can be administered to subjects in need thereof for about 30 minutes to about 24 hours, e.g., 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which can be repeated with a frequency of about every day to about every 4 days, e.g., every 1.5 days, every 2 days, every 3 days, or any other frequency from about every day to about every four days. The compositions may be provided to the subject one or more times, e.g., one time, twice, three times, or more than three times. In cases in which two or more different prime editing system components, e.g., two different polynucleotide constructs are administered to the subject (e.g., different components of the same prime editing system, or two different guide nucleic acids that are complementary to different sequences within the same or different target genes), the compositions may be administered simultaneously (e.g., as two polypeptides and/or nucleic acids). Alternatively, they may be provided sequentially, e.g., one composition being provided first, followed by a second composition.

### Pharmaceutical Compositions

Disclosed herein are pharmaceutical compositions comprising any of the prime editing composition components, for example, prime editors, fusion proteins, polynucleotides encoding prime editor polypeptides, PEgRNAs, and/or prime editing complexes described herein. Disclosed herein are pharmaceutical compositions comprising the edited cells, e.g., T cells, described herein. The pharmaceutical compositions of the present disclosure can be used to treat a disease, disorder, or a condition.

The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents, e.g., for specific delivery, increasing half-life, or other therapeutic compounds.

In some embodiments, a pharmaceutically-acceptable carrier comprises any vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, and physiologic pH).

Formulations of the pharmaceutical compositions described herein can be prepared by any method known or hereafter developed in the art of pharmacology. In general, such preparatory methods include the step of bringing the active ingredient(s) into association with an excipient and/or one or more other accessory ingredients, and then, if necessary and/or desirable, shaping and/or packaging the product into a desired single- or multi-dose unit. Pharmaceutical formulations can additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired.

### EXAMPLES

The following examples are provided for illustrative purposes only and are not intended to limit the scope of the claims provided herein.

### Example 1—General Methods

PEgRNA assembly: PERNA libraries may be assembled by one of three cloning methods: in the first method, pooled synthesized DNA oligos encoding the PEgRNA and flanking U6 expression plasmid homology regions may be cloned into U6 expression plasmids via Gibson cloning and sequencing of bacterial colonies via Sanger or Next-generation sequencing. In the second method, double-stranded linear DNA fragments encoding PEgRNA and homology sequences as above may be individually Gibson-cloned into U6 expression plasmids. In the third method, for each PEgRNA, separate oligos encoding a protospacer, a gRNA scaffold, and PERNA extension (PBS and RTT) may be ligated, and then cloned into a U6 expression plasmid as described in Anzalone et al., Nature. 2019 December; 576 (7785): 149-157. Bacterial colonies carrying sequence-verified plasmids may then be propagated in LB or TB. Plasmid DNA may be purified by minipreps for mammalian transfection. Alternatively, PEgRNAs may be chemically synthesized and modified.

HEK cell culture and transfection: HEK293T cells may be propagated in DMEM with 10% FBS. For 96 well-plates, the cells may be seeded at 10,000 cells per well 24 hours prior to transfection. For 48-well plates, the cells may be seeded at 30,000 cells per well 24 hours prior to transfection. Seeded cells may be transfected with Lipofectamine 2000 according to the manufacturer's directions with PEgRNAs and plasmid DNA encoding a prime editor fusion protein. Three days after transfection, genomic DNA may be harvested in lysis buffer for high throughput sequencing (e.g., using Miseq).

T cell culture and electroporation: T cells may be cultured in OpTimizer CTS T Cell Expansion SFM containing 5% CTS Immune Cell SR (ThermoFisher), L-Glutamine, Penicillin/Streptomycin (Lonza), 10 mM N-Acetyl-L-cysteine (Sigma-Aldrich, St. Louis, MO), 300 IU/mL IL-2, 5 ng/mL IL7, and 5 ng/mL IL-15 (PeproTech). T cells are activated with DynaBeads Human T-Activator CD3/CD28 (ThermoFisher) at a 2:1 bead: cell ratio for 48 hours prior to electroporation. After 48 hours of stimulation, DynaBeads may be magnetically removed, and T cells washed once with PBS prior to resuspension in electroporation buffer. For dual prime editing, mRNA encoding a prime editor fusion protein and chemically synthesized pegRNAs are suspended in electroporation buffer and mixed with T cells. For recombinase mediated donor DNA integration, mRNA encoding the recombinase (Bxb1) and the DNA donor may be similarly suspended in electroporation buffer and mixed and with T cells. Cells may be electroporated according to the manufacturer's protocol (MaxCyte). Following electroporation, T cells may be transferred to an empty well-plate and allowed to recover at 37° C., 5% CO₂ for 20 minutes. Subsequently, cells are suspended in basal medium (without serum replacement or cytokines) and incubated at 37° C., 5% CO₂ for 20 minutes before being suspended in complete T cell medium. Two days after electroporation, T cells may be transferred to G-Rex rapid expansion culture vessels. Genomic DNA may be extracted using Lucigen QuickExtraction Solution according to manufacturer's protocol for sequence analysis.

### Example 2—Dual Prime Editing of the TRAC Gene in HEK 293T Cells

PEgRNA pairs were designed for a dual prime editing strategy such that the protospacers of a PEgRNA pair flank the exon 1/intron 1 junction of the TRAC gene. The design would allow introduction of a recombinase recognition site at such genomic locations that a potential transgene (e.g. a transgene on a donor DNA) sequence integrated at the locations can be expressed using either an exogenous promoter (e.g., as part of the transgene) or the endogenous TRAC promoter. For 5′ PERNAs, 4 spacers were used with varying PBS lengths from 8 to 13nt. For 3′ PEgRNAs, 13 spacers were used with varying PBS lengths from 8 to 16nt. The RTT sequences were designed to replace the endogenous inter-nick TRAC sequence (the IND) with a 38 bp attB sequence, which is a substrate of a Bxb1 integrase: the 5′ PEgRNAs contain an RTT sequence according to SEQ ID NO: 27, and the 3′ PEgRNAs contain an RTT sequence according to SEQ ID NO: 107 (full complementarity between the 5′ RTT and 3′ RTT, each encoding the 38 bp attB sequence/reverse complement thereof). All of the PEgRNAs contained a gRNA core sequence according to SEQ ID NO: 590.

The PEgRNAs designed as above were assembled from U6 expression plasmids, and HEK293T cells were transfected with PEgRNA pairs and plasmid encoding a prime editor fusion protein as described in Example 1. 72 hours after transfection, genomic DNA was extracted by aspirating media, adding QuickExtract DNA Extraction solution (Lucigen, #QR9050) and incubating according to manufacturer's protocol for high-throughput sequencing. 467 PEgRNA pairs were tested to examine each 5′ PERNA spacer paired with each 3′ PEgRNA spacer having varying PBS lengths. Sequence numbers of the PEgRNAs and PEgRNA components, as well as editing efficiency and indels are summarized in Tables 31A-31D. As transcribed, the PEgRNAs in Tablesx1A-D will contain, from 5′ to 3′, the indicated PEgRNA sequence and a variable number of 3′ Us (e.g., up to 4 Us).

The editing efficiency and indel frequency scales in Tables 31A-31D are as follows. Editing efficiency: 0.3%-15%: +; 15%-29%: ++; 29%-43%: +++; 43%-58%: ++++. Indel frequency: 0.05%-4%: +; 4%-7%: ++; 7%-11%: +++; 11%-14%: ++++.

TABLE 31A.
Dual prime editing efficiency in HEK293T cells. All 5′ PEgRNAs have a spacer
sequence according to SEQ ID NO: 4. The 5′ PEgRNAs contain an RTT sequence according
to SEQ ID NO: 27. The 3′ PEgRNAs contain an RTT sequence according to SEQ ID NO: 107.

| | | 5′ | 3′ | | | 3′ | | | |
|---|---|---|---|---|---|---|---|---|---|
| 5′ PBS | 5′ | PEgRNA | Spacer | 3′ PBS | 3′ | PEgRNA | nick-to- | | |
| sequence | PBS | SEQ ID | SEQ ID | sequence | PBS | SEQ ID | nick | % | % |
| number | length | NO | NO | number | length | NO | distance | Editing | Indels |
| 11 | 9 | 43 | 177 | 184 | 9 | 217 | 74 | ++ | + |
| 11 | 9 | 43 | 177 | 186 | 11 | 220 | 74 | +++ | + |
| 11 | 9 | 43 | 177 | 188 | 13 | 221 | 74 | ++ | + |
| 13 | 11 | 44 | 177 | 184 | 9 | 217 | 74 | ++ | + |
| 13 | 11 | 44 | 177 | 186 | 11 | 220 | 74 | +++ | + |
| 13 | 11 | 44 | 177 | 188 | 13 | 221 | 74 | +++ | + |
| 15 | 13 | 49 | 177 | 184 | 9 | 217 | 74 | ++ | + |
| 15 | 13 | 49 | 177 | 186 | 11 | 220 | 74 | ++ | + |
| 15 | 13 | 49 | 177 | 188 | 13 | 221 | 74 | ++ | + |
| 11 | 9 | 43 | 233 | 239 | 8 | 251 | 64 | ++ | + |
| 11 | 9 | 43 | 233 | 241 | 10 | 252 | 64 | ++ | + |
| 11 | 9 | 43 | 233 | 243 | 12 | 254 | 64 | + | + |
| 13 | 11 | 44 | 233 | 239 | 8 | 251 | 64 | ++ | +++ |
| 13 | 11 | 44 | 233 | 241 | 10 | 252 | 64 | ++ | +++ |
| 13 | 11 | 44 | 233 | 243 | 12 | 254 | 64 | + | ++ |
| 15 | 13 | 49 | 233 | 239 | 8 | 251 | 64 | ++ | ++ |
| 15 | 13 | 49 | 233 | 241 | 10 | 252 | 64 | ++ | ++ |
| 15 | 13 | 49 | 233 | 243 | 12 | 254 | 64 | + | ++ |
| 11 | 9 | 43 | 260 | 267 | 9 | 278 | 32 | +++ | + |
| 11 | 9 | 43 | 260 | 269 | 11 | 279 | 32 | +++ | + |
| 11 | 9 | 43 | 260 | 271 | 13 | 281 | 32 | +++ | + |
| 13 | 11 | 44 | 260 | 267 | 9 | 278 | 32 | +++ | + |
| 13 | 11 | 44 | 260 | 269 | 11 | 279 | 32 | +++ | + |
| 13 | 11 | 44 | 260 | 271 | 13 | 281 | 32 | +++ | + |
| 15 | 13 | 49 | 260 | 267 | 9 | 278 | 32 | +++ | + |
| 15 | 13 | 49 | 260 | 269 | 11 | 279 | 32 | +++ | + |
| 15 | 13 | 49 | 260 | 271 | 13 | 281 | 32 | +++ | + |
| 11 | 9 | 43 | 287 | 293 | 8 | 305 | 69 | +++ | + |
| 11 | 9 | 43 | 287 | 295 | 10 | 306 | 69 | ++++ | + |
| 11 | 9 | 43 | 287 | 297 | 12 | 308 | 69 | +++ | + |
| 13 | 11 | 44 | 287 | 293 | 8 | 305 | 69 | ++++ | + |
| 13 | 11 | 44 | 287 | 295 | 10 | 306 | 69 | ++++ | + |
| 13 | 11 | 44 | 287 | 297 | 12 | 308 | 69 | ++++ | + |
| 15 | 13 | 49 | 287 | 293 | 8 | 305 | 69 | +++ | + |
| 15 | 13 | 49 | 287 | 295 | 10 | 306 | 69 | +++ | + |
| 15 | 13 | 49 | 287 | 297 | 12 | 308 | 69 | +++ | + |
| 11 | 9 | 43 | 314 | 321 | 9 | 332 | 38 | ++++ | + |
| 11 | 9 | 43 | 314 | 323 | 11 | 333 | 38 | ++++ | + |
| 11 | 9 | 43 | 314 | 325 | 13 | 335 | 38 | +++ | + |
| 13 | 11 | 44 | 314 | 321 | 9 | 332 | 38 | +++ | + |
| 13 | 11 | 44 | 314 | 323 | 11 | 333 | 38 | ++++ | + |
| 13 | 11 | 44 | 314 | 325 | 13 | 335 | 38 | ++++ | + |
| 15 | 13 | 49 | 314 | 321 | 9 | 332 | 38 | +++ | + |
| 15 | 13 | 49 | 314 | 323 | 11 | 333 | 38 | ++++ | + |
| 15 | 13 | 49 | 314 | 325 | 13 | 335 | 38 | +++ | + |
| 11 | 9 | 43 | 341 | 350 | 11 | 359 | 120 | + | ++ |
| 11 | 9 | 43 | 341 | 353 | 14 | 360 | 120 | + | +++ |
| 11 | 9 | 43 | 341 | 355 | 16 | 362 | 120 | + | ++++ |
| 13 | 11 | 44 | 341 | 350 | 1 | 359 | 120 | + | ++ |
| 13 | 11 | 44 | 341 | 353 | 14 | 360 | 120 | + | +++ |
| 13 | 11 | 44 | 341 | 355 | 16 | 362 | 120 | + | ++++ |
| 15 | 13 | 49 | 341 | 350 | 11 | 359 | 120 | + | ++ |
| 15 | 13 | 49 | 341 | 353 | 14 | 360 | 120 | + | ++ |
| 15 | 13 | 49 | 341 | 355 | 16 | 362 | 120 | + | +++ |
| 11 | 9 | 43 | 368 | 374 | 8 | 397 | 63 | ++ | + |
| 11 | 9 | 43 | 368 | 376 | 10 | 400 | 63 | ++ | + |
| 11 | 9 | 43 | 368 | 378 | 12 | 404 | 63 | + | + |
| 13 | 11 | 44 | 368 | 374 | 8 | 397 | 63 | ++ | ++ |
| 13 | 11 | 44 | 368 | 376 | 10 | 400 | 63 | ++ | ++ |
| 13 | 11 | 44 | 368 | 378 | 12 | 404 | 63 | + | +++ |
| 15 | 13 | 49 | 368 | 374 | 8 | 397 | 63 | ++ | + |
| 15 | 13 | 49 | 368 | 376 | 10 | 400 | 63 | ++ | ++ |
| 15 | 13 | 49 | 368 | 378 | 12 | 404 | 63 | + | ++ |
| 11 | 9 | 43 | 414 | 421 | 9 | 432 | 26 | +++ | + |
| 11 | 9 | 43 | 414 | 424 | 12 | 433 | 26 | +++ | + |
| 11 | 9 | 43 | 414 | 426 | 14 | 435 | 26 | ++ | + |
| 13 | 11 | 44 | 414 | 421 | 9 | 432 | 26 | +++ | + |
| 13 | 11 | 44 | 414 | 424 | 12 | 433 | 26 | +++ | ++ |
| 13 | 11 | 44 | 414 | 426 | 14 | 435 | 26 | ++ | ++ |
| 15 | 13 | 49 | 414 | 421 | 9 | 432 | 26 | ++ | + |
| 15 | 13 | 49 | 414 | 424 | 12 | 433 | 26 | ++ | + |
| 15 | 13 | 49 | 414 | 426 | 14 | 435 | 26 | ++ | + |
| 11 | 9 | 43 | 441 | 449 | 10 | 459 | 47 | ++ | + |
| 11 | 9 | 43 | 441 | 451 | 12 | 460 | 47 | ++ | + |
| 11 | 9 | 43 | 441 | 453 | 14 | 462 | 47 | ++ | + |
| 13 | 11 | 44 | 441 | 449 | 10 | 459 | 47 | ++ | + |
| 13 | 11 | 44 | 441 | 451 | 12 | 460 | 47 | ++ | + |
| 13 | 11 | 44 | 441 | 453 | 14 | 462 | 47 | ++ | + |
| 15 | 13 | 49 | 441 | 449 | 10 | 459 | 47 | + | + |
| 15 | 13 | 49 | 441 | 451 | 12 | 460 | 47 | ++ | + |
| 15 | 13 | 49 | 441 | 453 | 14 | 462 | 47 | ++ | + |
| 11 | 9 | 43 | 468 | 476 | 10 | 486 | 101 | +++ | + |
| 11 | 9 | 43 | 468 | 478 | 12 | 487 | 101 | ++++ | + |
| 11 | 9 | 43 | 468 | 480 | 14 | 489 | 101 | ++++ | |
| 13 | 11 | 44 | 468 | 476 | 10 | 486 | 101 | +++ | + |
| 13 | 11 | 44 | 468 | 478 | 12 | 487 | 101 | ++++ | + |
| 13 | 11 | 44 | 468 | 480 | 14 | 489 | 101 | +++ | + |
| 15 | 13 | 49 | 468 | 476 | 10 | 486 | 101 | +++ | + |
| 15 | 13 | 49 | 468 | 478 | 12 | 487 | 101 | +++ | |
| 15 | 13 | 49 | 468 | 480 | 14 | 489 | 101 | +++ | + |
| 11 | 9 | 43 | 495 | 503 | 10 | 513 | 46 | +++ | + |
| 11 | 9 | 43 | 495 | 505 | 12 | 514 | 46 | +++ | + |
| 11 | 9 | 43 | 495 | 508 | 15 | 516 | 46 | +++ | + |
| 13 | 11 | 44 | 495 | 503 | 10 | 513 | 46 | +++ | + |
| 13 | 11 | 44 | 495 | 505 | 12 | 514 | 46 | ++++ | + |
| 13 | 11 | 44 | 495 | 508 | 15 | 516 | 46 | ++++ | + |
| 15 | 13 | 49 | 495 | 503 | 10 | 513 | 46 | ++ | + |
| 15 | 13 | 49 | 495 | 505 | 12 | 514 | 46 | +++ | + |
| 15 | 13 | 49 | 495 | 508 | 15 | 516 | 46 | +++ | + |
| 11 | 9 | 43 | 522 | 529 | 9 | 547 | 70 | + | + |
| 11 | 9 | 43 | 522 | 531 | 11 | 550 | 70 | + | + |
| 11 | 9 | 43 | 522 | 533 | 13 | 554 | 70 | + | + |
| 13 | 11 | 44 | 522 | 529 | 9 | 547 | 70 | + | + |
| 13 | 11 | 44 | 522 | 531 | 11 | 550 | 70 | + | + |
| 13 | 11 | 44 | 522 | 533 | 13 | 554 | 70 | + | + |
| 15 | 13 | 49 | 522 | 529 | 9 | 547 | 70 | + | + |
| 15 | 13 | 49 | 522 | 531 | 11 | 550 | 70 | + | + |
| 15 | 13 | 49 | 522 | 533 | 13 | 554 | 70 | + | + |
| 11 | 9 | 43 | 566 | 573 | 9 | 584 | 31 | + | + |
| 11 | 9 | 43 | 566 | 575 | 11 | 585 | 31 | + | + |
| 11 | 9 | 43 | 566 | 577 | 13 | 587 | 31 | + | + |
| 13 | 11 | 44 | 566 | 573 | 9 | 584 | 31 | + | + |
| 13 | 11 | 44 | 566 | 575 | 11 | 585 | 31 | + | + |
| 13 | 11 | 44 | 566 | 577 | 13 | 587 | 31 | + | + |
| 15 | 13 | 49 | 566 | 573 | 9 | 584 | 31 | + | + |
| 15 | 13 | 49 | 566 | 575 | 11 | 585 | 31 | + | + |
| 15 | 13 | 49 | 566 | 577 | 13 | 587 | 31 | + | + |

TABLE 31B
Dual prime editing efficiency in HEK293T cells. All 5′ PEgRNAs have a spacer sequence
according to SEQ ID NO: 61. The 5′ PEgRNAs contain an RTT sequence according to
SEQ ID NO: 27. The 3′ PEgRNAs contain an RTT sequence according to SEQ ID NO:
107.

| | | 5′ | 3′ | | | 3′ | | | |
|---|---|---|---|---|---|---|---|---|---|
| 5′ PBS | 5′ | PEgRNA | Spacer | 3′ PBS | 3′ | PEgRNA | nick-to- | | |
| sequence | PBS | SEQ | SEQ ID | sequence | PBS | SEQ | nick | % | % |
| number | length | ID NO | NO | number | length | ID NO | distance | Editing | Indels |
| 67 | 8 | 79 | 177 | 184 | 9 | 217 | 75 | + | + |
| 67 | 8 | 79 | 177 | 186 | 11 | 220 | 75 | ++ | + |
| 67 | 8 | 79 | 177 | 188 | 13 | 221 | 75 | ++ | + |
| 69 | 10 | 80 | 177 | 184 | 9 | 217 | 75 | ++ | + |
| 69 | 10 | 80 | 177 | 186 | 11 | 220 | 75 | ++ | + |
| 69 | 10 | 80 | 177 | 188 | 13 | 221 | 75 | ++ | + |
| 71 | 12 | 82 | 177 | 184 | 9 | 217 | 75 | + | + |
| 71 | 12 | 82 | 177 | 186 | 11 | 220 | 75 | ++ | + |
| 71 | 12 | 82 | 177 | 188 | 13 | 221 | 75 | + | + |
| 67 | 8 | 79 | 233 | 239 | 8 | 251 | 65 | + | + |
| 67 | 8 | 79 | 233 | 241 | 10 | 252 | 65 | + | + |
| 67 | 8 | 79 | 233 | 243 | 12 | 254 | 65 | + | + |
| 69 | 10 | 80 | 233 | 239 | 8 | 251 | 65 | + | + |
| 69 | 10 | 80 | 233 | 243 | 12 | 254 | 65 | + | + |
| 71 | 12 | 82 | 233 | 239 | 8 | 251 | 65 | + | + |
| 71 | 12 | 82 | 233 | 241 | 10 | 252 | 65 | + | + |
| 71 | 12 | 82 | 233 | 243 | 12 | 254 | 65 | + | + |
| 67 | 8 | 79 | 260 | 267 | 9 | 278 | 33 | ++ | + |
| 67 | 8 | 79 | 260 | 269 | 11 | 279 | 33 | ++ | + |
| 67 | 8 | 79 | 260 | 271 | 13 | 281 | 33 | ++ | + |
| 69 | 10 | 80 | 260 | 267 | 9 | 278 | 33 | +++ | + |
| 69 | 10 | 80 | 260 | 269 | 11 | 279 | 33 | ++ | + |
| 69 | 10 | 80 | 260 | 271 | 13 | 281 | 33 | +++ | + |
| 71 | 12 | 82 | 260 | 267 | 9 | 278 | 33 | ++ | + |
| 71 | 12 | 82 | 260 | 269 | 11 | 279 | 33 | ++ | + |
| 71 | 12 | 82 | 260 | 271 | 13 | 281 | 33 | ++ | + |
| 67 | 8 | 79 | 287 | 293 | 8 | 305 | 70 | ++ | + |
| 67 | 8 | 79 | 287 | 295 | 10 | 306 | 70 | ++ | + |
| 67 | 8 | 79 | 287 | 297 | 12 | 308 | 70 | ++ | + |
| 69 | 10 | 80 | 287 | 293 | 8 | 305 | 70 | ++ | + |
| 69 | 10 | 80 | 287 | 295 | 10 | 306 | 70 | +++ | + |
| 69 | 10 | 80 | 287 | 297 | 12 | 308 | 70 | +++ | + |
| 71 | 12 | 82 | 287 | 293 | 8 | 305 | 70 | ++ | + |
| 71 | 12 | 82 | 287 | 295 | 10 | 306 | 70 | ++ | + |
| 71 | 12 | 82 | 287 | 297 | 12 | 308 | 70 | ++ | + |
| 67 | 8 | 79 | 314 | 321 | 9 | 332 | 39 | ++ | + |
| 67 | 8 | 79 | 314 | 323 | 11 | 333 | 39 | +++ | + |
| 67 | 8 | 79 | 314 | 325 | 13 | 335 | 39 | ++ | + |
| 69 | 10 | 80 | 314 | 321 | 9 | 332 | 39 | +++ | + |
| 69 | 10 | 80 | 314 | 323 | 11 | 333 | 39 | ++ | + |
| 69 | 10 | 80 | 314 | 325 | 13 | 335 | 39 | ++ | + |
| 71 | 12 | 82 | 314 | 321 | 9 | 332 | 39 | ++ | + |
| 71 | 12 | 82 | 314 | 323 | 11 | 333 | 39 | ++ | + |
| 71 | 12 | 82 | 314 | 325 | 13 | 335 | 39 | ++ | + |
| 67 | 8 | 79 | 341 | 350 | 11 | 359 | 121 | + | ++ |
| 67 | 8 | 79 | 341 | 353 | 14 | 360 | 121 | + | ++ |
| 67 | 8 | 79 | 341 | 355 | 16 | 362 | 121 | ++ | + |
| 69 | 10 | 80 | 341 | 350 | 11 | 359 | 121 | + | ++ |
| 69 | 10 | 80 | 341 | 353 | 14 | 360 | 121 | + | ++ |
| 69 | 10 | 80 | 341 | 355 | 16 | 362 | 121 | + | +++ |
| 71 | 12 | 82 | 341 | 350 | 11 | 359 | 121 | + | + |
| 71 | 12 | 82 | 341 | 353 | 14 | 360 | 121 | + | ++ |
| 71 | 12 | 82 | 341 | 355 | 16 | 362 | 121 | + | ++ |
| 67 | 8 | 79 | 368 | 374 | 8 | 397 | 64 | + | + |
| 67 | 8 | 79 | 368 | 376 | 10 | 400 | 64 | + | + |
| 67 | 8 | 79 | 368 | 378 | 12 | 404 | 64 | + | + |
| 69 | 10 | 80 | 368 | 374 | 8 | 397 | 64 | ++ | + |
| 69 | 10 | 80 | 368 | 376 | 10 | 400 | 64 | + | + |
| 69 | 10 | 80 | 368 | 378 | 12 | 404 | 64 | + | + |
| 71 | 12 | 82 | 368 | 374 | 8 | 397 | 64 | + | + |
| 71 | 12 | 82 | 368 | 376 | 10 | 400 | 64 | + | + |
| 71 | 12 | 82 | 368 | 378 | 12 | 404 | 64 | + | + |
| 67 | 8 | 79 | 414 | 421 | 9 | 432 | 27 | + | + |
| 67 | 8 | 79 | 414 | 424 | 12 | 433 | 27 | ++ | + |
| 67 | 8 | 79 | 414 | 426 | 14 | 435 | 27 | + | + |
| 69 | 10 | 80 | 414 | 421 | 9 | 432 | 27 | ++ | + |
| 69 | 10 | 80 | 414 | 424 | 12 | 433 | 27 | + | + |
| 69 | 10 | 80 | 414 | 426 | 14 | 435 | 27 | + | + |
| 71 | 12 | 82 | 414 | 421 | 9 | 432 | 27 | + | + |
| 71 | 12 | 82 | 414 | 424 | 12 | 433 | 27 | + | + |
| 71 | 12 | 82 | 414 | 426 | 14 | 435 | 27 | + | + |
| 67 | 8 | 79 | 441 | 449 | 10 | 459 | 48 | + | + |
| 67 | 8 | 79 | 441 | 451 | 12 | 460 | 48 | + | + |
| 67 | 8 | 79 | 441 | 453 | 14 | 462 | 48 | + | + |
| 69 | 10 | 80 | 441 | 449 | 10 | 459 | 48 | + | + |
| 69 | 10 | 80 | 441 | 451 | 12 | 460 | 48 | + | + |
| 69 | 10 | 80 | 441 | 453 | 14 | 462 | 48 | ++ | + |
| 71 | 12 | 82 | 441 | 449 | 10 | 459 | 48 | + | + |
| 71 | 12 | 82 | 441 | 451 | 12 | 460 | 48 | + | + |
| 71 | 12 | 82 | 441 | 453 | 14 | 462 | 48 | + | + |
| 67 | 8 | 79 | 468 | 476 | 10 | 486 | 102 | ++ | + |
| 67 | 8 | 79 | 468 | 478 | 12 | 487 | 102 | ++ | + |
| 67 | 8 | 79 | 468 | 480 | 14 | 489 | 102 | ++ | + |
| 69 | 10 | 80 | 468 | 476 | 10 | 486 | 102 | +++ | + |
| 69 | 10 | 80 | 468 | 478 | 12 | 487 | 102 | +++ | + |
| 69 | 10 | 80 | 468 | 480 | 14 | 489 | 102 | ++ | + |
| 71 | 12 | 82 | 468 | 476 | 10 | 486 | 102 | ++ | + |
| 71 | 12 | 82 | 468 | 478 | 12 | 487 | 102 | ++ | + |
| 71 | 12 | 82 | 468 | 480 | 14 | 489 | 102 | ++ | + |
| 67 | 8 | 79 | 495 | 503 | 10 | 513 | 47 | ++ | + |
| 67 | 8 | 79 | 495 | 505 | 12 | 514 | 47 | ++ | + |
| 67 | 8 | 79 | 495 | 508 | 15 | 516 | 47 | ++ | + |
| 69 | 10 | 80 | 495 | 503 | 10 | 513 | 47 | +++ | + |
| 69 | 10 | 80 | 495 | 505 | 12 | 514 | 47 | ++ | + |
| 69 | 10 | 80 | 495 | 508 | 15 | 516 | 47 | + | + |
| 71 | 12 | 82 | 495 | 503 | 10 | 513 | 47 | + | + |
| 71 | 12 | 82 | 495 | 505 | 12 | 514 | 47 | ++ | + |
| 71 | 12 | 82 | 495 | 508 | 15 | 516 | 47 | ++ | + |
| 67 | 8 | 79 | 522 | 529 | 9 | 547 | 71 | ++ | + |
| 67 | 8 | 79 | 522 | 531 | 11 | 550 | 71 | ++ | + |
| 67 | 8 | 79 | 522 | 533 | 13 | 554 | 71 | + | + |
| 69 | 10 | 80 | 522 | 529 | 9 | 547 | 71 | ++ | + |
| 69 | 10 | 80 | 522 | 531 | 11 | 550 | 71 | ++ | + |
| 69 | 10 | 80 | 522 | 533 | 13 | 554 | 71 | ++ | + |
| 71 | 12 | 82 | 522 | 529 | 9 | 547 | 71 | + | + |
| 71 | 12 | 82 | 522 | 531 | 11 | 550 | 71 | ++ | + |
| 71 | 12 | 82 | 522 | 533 | 13 | 554 | 71 | + | + |
| 67 | 8 | 79 | 566 | 573 | 9 | 584 | 32 | + | + |
| 67 | 8 | 79 | 566 | 575 | 11 | 585 | 32 | + | + |
| 67 | 8 | 79 | 566 | 577 | 13 | 587 | 32 | + | + |
| 69 | 10 | 80 | 566 | 573 | 9 | 584 | 32 | + | + |
| 69 | 10 | 80 | 566 | 575 | 11 | 585 | 32 | + | + |
| 69 | 10 | 80 | 566 | 577 | 13 | 587 | 32 | +++ | + |
| 71 | 12 | 82 | 566 | 573 | 9 | 584 | 32 | + | + |
| 71 | 12 | 82 | 566 | 575 | 11 | 585 | 32 | + | + |
| 71 | 12 | 82 | 566 | 577 | 13 | 587 | 32 | + | + |

TABLE 31C
Dual prime editing efficiency in HEK293T cells. All 5′ PEgRNAs have a spacer sequence
according to SEQ ID NO: 88. The 5′ PEgRNAs contain an RTT sequence according to
SEQ ID NO: 27. The 3′ PEgRNAs contain an RTT sequence according to SEQ ID NO: 107.

| | | 5′ | 3′ | | | 3′ | | | |
|---|---|---|---|---|---|---|---|---|---|
| 5′ PBS | 5′ | PEgRNA | Spacer | 3′ PBS | 3′ | PEgRNA | nick-to- | | |
| sequence | PBS | SEQ | SEQ ID | sequence | PBS | SEQ | nick | % | % |
| number | length | ID NO | NO | number | length | ID NO | distance | Editing | Indels |
| 94 | 8 | 122 | 177 | 184 | 9 | 217 | 83 | +++ | + |
| 94 | 8 | 122 | 177 | 186 | 11 | 220 | 83 | ++++ | + |
| 94 | 8 | 122 | 177 | 188 | 13 | 221 | 83 | +++ | + |
| 96 | 10 | 126 | 177 | 184 | 9 | 217 | 83 | ++ | + |
| 96 | 10 | 126 | 177 | 186 | 11 | 220 | 83 | +++ | + |
| 96 | 10 | 126 | 177 | 188 | 13 | 221 | 83 | +++ | + |
| 99 | 13 | 134 | 177 | 184 | 9 | 217 | 83 | ++ | + |
| 99 | 13 | 134 | 177 | 186 | 11 | 220 | 83 | +++ | + |
| 99 | 13 | 134 | 177 | 188 | 13 | 221 | 83 | +++ | + |
| 94 | 8 | 122 | 233 | 239 | 8 | 251 | 73 | +++ | + |
| 94 | 8 | 122 | 233 | 241 | 10 | 252 | 73 | +++ | + |
| 94 | 8 | 122 | 233 | 243 | 12 | 254 | 73 | ++ | + |
| 96 | 10 | 126 | 233 | 239 | 8 | 251 | 73 | ++ | + |
| 96 | 10 | 126 | 233 | 241 | 10 | 252 | 73 | ++ | + |
| 96 | 10 | 126 | 233 | 243 | 12 | 254 | 73 | ++ | + |
| 99 | 13 | 134 | 233 | 239 | 8 | 251 | 73 | ++ | + |
| 99 | 13 | 134 | 233 | 241 | 10 | 252 | 73 | ++ | + |
| 99 | 13 | 134 | 233 | 243 | 12 | 254 | 73 | ++ | + |
| 94 | 8 | 122 | 260 | 267 | 9 | 278 | 41 | ++++ | + |
| 94 | 8 | 122 | 260 | 269 | 11 | 279 | 41 | ++++ | + |
| 94 | 8 | 122 | 260 | 271 | 13 | 281 | 41 | ++++ | + |
| 96 | 10 | 126 | 260 | 267 | 9 | 278 | 41 | ++++ | + |
| 96 | 10 | 126 | 260 | 269 | 11 | 279 | 41 | ++++ | + |
| 96 | 10 | 126 | 260 | 271 | 13 | 281 | 41 | +++ | + |
| 99 | 13 | 134 | 260 | 267 | 9 | 278 | 41 | +++ | + |
| 99 | 13 | 134 | 260 | 269 | 11 | 279 | 41 | +++ | + |
| 99 | 13 | 134 | 260 | 271 | 13 | 281 | 41 | +++ | + |
| 94 | 8 | 122 | 287 | 293 | 8 | 305 | 78 | ++++ | + |
| 94 | 8 | 122 | 287 | 295 | 10 | 306 | 78 | ++++ | + |
| 94 | 8 | 122 | 287 | 297 | 12 | 308 | 78 | ++++ | + |
| 96 | 10 | 126 | 287 | 293 | 8 | 305 | 78 | +++ | + |
| 96 | 10 | 126 | 287 | 295 | 10 | 306 | 78 | ++++ | + |
| 96 | 10 | 126 | 287 | 297 | 12 | 308 | 78 | ++++ | + |
| 99 | 13 | 134 | 287 | 293 | 8 | 305 | 78 | +++ | + |
| 99 | 13 | 134 | 287 | 295 | 10 | 306 | 78 | ++++ | + |
| 99 | 13 | 134 | 287 | 297 | 12 | 308 | 78 | +++ | + |
| 94 | 8 | 122 | 314 | 321 | 9 | 332 | 47 | ++++ | + |
| 94 | 8 | 122 | 314 | 323 | 11 | 333 | 47 | ++++ | + |
| 94 | 8 | 122 | 314 | 325 | 13 | 335 | 47 | ++++ | + |
| 96 | 10 | 126 | 314 | 321 | 9 | 332 | 47 | ++++ | + |
| 96 | 10 | 126 | 314 | 323 | 11 | 333 | 47 | ++++ | + |
| 96 | 10 | 126 | 314 | 325 | 13 | 335 | 47 | +++ | + |
| 99 | 13 | 134 | 314 | 321 | 9 | 332 | 47 | ++++ | + |
| 99 | 13 | 134 | 314 | 323 | 11 | 333 | 47 | ++++ | + |
| 99 | 13 | 134 | 314 | 325 | 13 | 335 | 47 | +++ | + |
| 94 | 8 | 122 | 341 | 350 | 11 | 359 | 129 | + | ++ |
| 94 | 8 | 122 | 341 | 353 | 14 | 360 | 129 | + | ++++ |
| 94 | 8 | 122 | 341 | 355 | 16 | 362 | 129 | + | ++++ |
| 96 | 10 | 126 | 341 | 350 | 11 | 359 | 129 | + | ++ |
| 96 | 10 | 126 | 341 | 353 | 14 | 360 | 129 | + | +++ |
| 96 | 10 | 126 | 341 | 355 | 16 | 362 | 129 | + | +++ |
| 99 | 13 | 134 | 341 | 350 | 11 | 359 | 129 | + | ++ |
| 99 | 13 | 134 | 341 | 353 | 14 | 360 | 129 | + | +++ |
| 99 | 13 | 134 | 341 | 355 | 16 | 362 | 129 | + | ++++ |
| 94 | 8 | 122 | 368 | 374 | 8 | 397 | 72 | +++ | + |
| 94 | 8 | 122 | 368 | 376 | 10 | 400 | 72 | +++ | ++ |
| 94 | 8 | 122 | 368 | 378 | 12 | 404 | 72 | + | ++ |
| 96 | 10 | 126 | 368 | 374 | 8 | 397 | 72 | ++ | + |
| 96 | 10 | 126 | 368 | 376 | 10 | 400 | 72 | ++ | + |
| 96 | 10 | 126 | 368 | 378 | 12 | 404 | 72 | + | ++ |
| 99 | 13 | 134 | 368 | 374 | 8 | 397 | 72 | ++ | + |
| 99 | 13 | 134 | 368 | 376 | 10 | 400 | 72 | ++ | + |
| 99 | 13 | 134 | 368 | 378 | 12 | 404 | 72 | + | ++ |
| 94 | 8 | 122 | 414 | 421 | 9 | 432 | 35 | +++ | + |
| 94 | 8 | 122 | 414 | 424 | 12 | 433 | 35 | +++ | + |
| 94 | 8 | 122 | 414 | 426 | 14 | 435 | 35 | +++ | ++ |
| 96 | 10 | 126 | 414 | 421 | 9 | 432 | 35 | +++ | + |
| 96 | 10 | 126 | 414 | 424 | 12 | 433 | 35 | +++ | + |
| 96 | 10 | 126 | 414 | 426 | 14 | 435 | 35 | ++ | + |
| 99 | 13 | 134 | 414 | 421 | 9 | 432 | 35 | +++ | + |
| 99 | 13 | 134 | 414 | 424 | 12 | 433 | 35 | +++ | + |
| 99 | 13 | 134 | 414 | 426 | 14 | 435 | 35 | ++ | + |
| 94 | 8 | 122 | 441 | 449 | 10 | 459 | 56 | ++ | + |
| 94 | 8 | 122 | 441 | 451 | 12 | 460 | 56 | ++ | + |
| 94 | 8 | 122 | 441 | 453 | 14 | 462 | 56 | ++ | + |
| 96 | 10 | 126 | 441 | 449 | 10 | 459 | 56 | ++ | + |
| 96 | 10 | 126 | 441 | 451 | 12 | 460 | 56 | ++ | + |
| 96 | 10 | 126 | 441 | 453 | 14 | 462 | 56 | ++ | |
| 99 | 13 | 134 | 441 | 449 | 10 | 459 | 56 | ++ | + |
| 99 | 13 | 134 | 441 | 451 | 12 | 460 | 56 | ++ | + |
| 99 | 13 | 134 | 441 | 453 | 14 | 462 | 56 | ++ | + |
| 94 | 8 | 122 | 468 | 476 | 10 | 486 | 110 | ++++ | + |
| 94 | 8 | 122 | 468 | 478 | 12 | 487 | 110 | ++++ | + |
| 94 | 8 | 122 | 468 | 480 | 14 | 489 | 110 | ++++ | + |
| 96 | 10 | 126 | 468 | 476 | 10 | 486 | 110 | ++++ | + |
| 96 | 10 | 126 | 468 | 478 | 12 | 487 | 110 | ++++ | + |
| 96 | 10 | 126 | 468 | 480 | 14 | 489 | 110 | ++++ | + |
| 99 | 13 | 134 | 468 | 476 | 10 | 486 | 110 | ++++ | + |
| 99 | 13 | 134 | 468 | 478 | 12 | 487 | 110 | ++++ | + |
| 99 | 13 | 134 | 468 | 480 | 14 | 489 | 110 | ++++ | + |
| 94 | 8 | 122 | 495 | 503 | 10 | 513 | 55 | +++ | + |
| 94 | 8 | 122 | 495 | 505 | 12 | 514 | 55 | ++++ | + |
| 94 | 8 | 122 | 495 | 508 | 15 | 516 | 55 | ++++ | + |
| 96 | 10 | 126 | 495 | 503 | 10 | 513 | 55 | +++ | + |
| 96 | 10 | 126 | 495 | 505 | 12 | 514 | 55 | ++++ | + |
| 96 | 10 | 126 | 495 | 508 | 15 | 516 | 55 | ++++ | + |
| 99 | 13 | 134 | 495 | 503 | 10 | 513 | 55 | +++ | + |
| 99 | 13 | 134 | 495 | 505 | 12 | 514 | 55 | +++ | + |
| 99 | 13 | 134 | 495 | 508 | 15 | 516 | 55 | +++ | + |
| 94 | 8 | 122 | 522 | 529 | 9 | 547 | 79 | +++ | + |
| 94 | 8 | 122 | 522 | 531 | 11 | 550 | 79 | ++++ | + |
| 94 | 8 | 122 | 522 | 533 | 13 | 554 | 79 | +++ | + |
| 96 | 10 | 126 | 522 | 529 | 9 | 547 | 79 | ++ | + |
| 96 | 10 | 126 | 522 | 531 | 11 | 550 | 79 | ++++ | + |
| 96 | 10 | 126 | 522 | 533 | 13 | 554 | 79 | +++ | + |
| 99 | 13 | 134 | 522 | 529 | 9 | 547 | 79 | ++ | + |
| 99 | 13 | 134 | 522 | 531 | 11 | 550 | 79 | +++ | + |
| 99 | 13 | 134 | 522 | 533 | 13 | 554 | 79 | +++ | + |
| 94 | 8 | 122 | 566 | 573 | 9 | 584 | 40 | + | + |
| 94 | 8 | 122 | 566 | 575 | 11 | 585 | 40 | ++ | + |
| 94 | 8 | 122 | 566 | 577 | 13 | 587 | 40 | + | + |
| 96 | 10 | 126 | 566 | 573 | 9 | 584 | 40 | + | + |
| 96 | 10 | 126 | 566 | 575 | 11 | 585 | 40 | + | + |
| 96 | 10 | 126 | 566 | 577 | 13 | 587 | 40 | + | + |
| 99 | 13 | 134 | 566 | 573 | 9 | 584 | 40 | + | + |
| 99 | 13 | 134 | 566 | 575 | 11 | 585 | 40 | + | + |
| 99 | 13 | 134 | 566 | 577 | 13 | 587 | 40 | + | + |

TABLE 31D
Dual prime editing efficiency in HEK293T cells. All 5′ PEgRNAs have a spacer sequence
according to SEQ ID NO: 150. The 5′ PEgRNAs contain an RTT sequence according to
SEQ ID NO: 27. The 3′ PEgRNAs contain an RTT sequence according to SEQ ID NO: 107

| | | 5′ | 3′ | | | 3′ | | | |
|---|---|---|---|---|---|---|---|---|---|
| 5′ PBS | 5′ | PEgRNA | Spacer | 3′ PBS | 3′ | PEgRNA | nick-to- | | |
| sequence | PBS | SEQ | SEQ ID | sequence | PBS | SEQ | nick | % | % |
| number | length | ID NO | NO | number | length | ID NO | distance | Editing | Indels |
| 156 | 8 | 168 | 177 | 184 | 9 | 217 | 84 | ++ | + |
| 156 | 8 | 168 | 177 | 186 | 11 | 220 | 84 | ++ | + |
| 156 | 8 | 168 | 177 | 188 | 13 | 221 | 84 | ++ | + |
| 158 | 10 | 169 | 177 | 184 | 9 | 217 | 84 | + | + |
| 158 | 10 | 169 | 177 | 186 | 11 | 220 | 84 | ++ | + |
| 158 | 10 | 169 | 177 | 188 | 13 | 221 | 84 | ++ | + |
| 160 | 12 | 171 | 177 | 184 | 9 | 217 | 84 | + | + |
| 160 | 12 | 171 | 177 | 186 | 11 | 220 | 84 | ++ | + |
| 160 | 12 | 171 | 177 | 188 | 13 | 221 | 84 | + | + |
| 156 | 8 | 168 | 233 | 239 | 8 | 251 | 74 | ++ | + |
| 156 | 8 | 168 | 233 | 241 | 10 | 252 | 74 | ++ | + |
| 156 | 8 | 168 | 233 | 243 | 12 | 254 | 74 | + | + |
| 158 | 10 | 169 | 233 | 239 | 8 | 251 | 74 | + | + |
| 158 | 10 | 169 | 233 | 241 | 10 | 252 | 74 | + | + |
| 158 | 10 | 169 | 233 | 243 | 12 | 254 | 74 | + | + |
| 160 | 12 | 171 | 233 | 239 | 8 | 251 | 74 | + | + |
| 160 | 12 | 171 | 233 | 241 | 10 | 252 | 74 | + | + |
| 160 | 12 | 171 | 233 | 243 | 12 | 254 | 74 | + | + |
| 156 | 8 | 168 | 260 | 267 | 9 | 278 | 42 | ++ | + |
| 156 | 8 | 168 | 260 | 269 | 11 | 279 | 42 | +++ | + |
| 156 | 8 | 168 | 260 | 271 | 13 | 281 | 42 | ++ | + |
| 158 | 10 | 169 | 260 | 267 | 9 | 278 | 42 | ++ | + |
| 158 | 10 | 169 | 260 | 269 | 11 | 279 | 42 | ++ | + |
| 158 | 10 | 169 | 260 | 271 | 13 | 281 | 42 | ++ | + |
| 160 | 12 | 171 | 260 | 267 | 9 | 278 | 42 | ++ | + |
| 160 | 12 | 171 | 260 | 269 | 11 | 279 | 42 | ++ | + |
| 160 | 12 | 171 | 260 | 271 | 13 | 281 | 42 | ++ | + |
| 156 | 8 | 168 | 287 | 293 | 8 | 305 | 79 | +++ | + |
| 156 | 8 | 168 | 287 | 295 | 10 | 306 | 79 | +++ | + |
| 156 | 8 | 168 | 287 | 297 | 12 | 308 | 79 | ++ | + |
| 158 | 10 | 169 | 287 | 293 | 8 | 305 | 79 | ++ | + |
| 158 | 10 | 169 | 287 | 295 | 10 | 306 | 79 | ++ | + |
| 158 | 10 | 169 | 287 | 297 | 12 | 308 | 79 | ++ | + |
| 160 | 12 | 171 | 287 | 293 | 8 | 305 | 79 | ++ | + |
| 160 | 12 | 171 | 287 | 295 | 10 | 306 | 79 | ++ | + |
| 160 | 12 | 171 | 287 | 297 | 12 | 308 | 79 | ++ | + |
| 156 | 8 | 168 | 314 | 321 | 9 | 332 | 48 | +++ | + |
| 156 | 8 | 168 | 314 | 323 | 11 | 333 | 48 | +++ | |
| 156 | 8 | 168 | 314 | 325 | 13 | 335 | 48 | +++ | + |
| 158 | 10 | 169 | 314 | 321 | 9 | 332 | 48 | +++ | + |
| 158 | 10 | 169 | 314 | 323 | 11 | 333 | 48 | +++ | + |
| 158 | 10 | 169 | 314 | 325 | 13 | 335 | 48 | ++ | + |
| 160 | 12 | 171 | 314 | 321 | 9 | 332 | 48 | ++ | + |
| 160 | 12 | 171 | 314 | 323 | 11 | 333 | 48 | ++ | + |
| 160 | 12 | 171 | 314 | 325 | 13 | 335 | 48 | ++ | + |
| 156 | 8 | 168 | 341 | 350 | 11 | 359 | 130 | + | + |
| 156 | 8 | 168 | 341 | 353 | 14 | 360 | 130 | + | ++ |
| 156 | 8 | 168 | 341 | 355 | 16 | 362 | 130 | + | ++ |
| 158 | 10 | 169 | 341 | 350 | 11 | 359 | 130 | + | + |
| 158 | 10 | 169 | 341 | 353 | 14 | 360 | 130 | + | ++ |
| 158 | 10 | 169 | 341 | 355 | 16 | 362 | 130 | + | ++ |
| 160 | 12 | 171 | 341 | 350 | 11 | 359 | 130 | + | + |
| 160 | 12 | 171 | 341 | 353 | 14 | 360 | 130 | + | + |
| 160 | 12 | 171 | 341 | 355 | 16 | 362 | 130 | + | + |
| 156 | 8 | 168 | 368 | 374 | 8 | 397 | 73 | ++ | + |
| 156 | 8 | 168 | 368 | 376 | 10 | 400 | 73 | ++ | + |
| 156 | 8 | 168 | 368 | 378 | 12 | 404 | 73 | + | + |
| 158 | 10 | 169 | 368 | 374 | 8 | 397 | 73 | + | + |
| 158 | 10 | 169 | 368 | 376 | 10 | 400 | 73 | + | + |
| 158 | 10 | 169 | 368 | 378 | 12 | 404 | 73 | + | + |
| 160 | 12 | 171 | 368 | 374 | 8 | 397 | 73 | + | + |
| 160 | 12 | 171 | 368 | 376 | 10 | 400 | 73 | + | + |
| 160 | 12 | 171 | 368 | 378 | 12 | 404 | 73 | + | + |
| 156 | 8 | 168 | 414 | 421 | 9 | 432 | 36 | ++ | + |
| 156 | 8 | 168 | 414 | 424 | 12 | 433 | 36 | ++ | + |
| 156 | 8 | 168 | 414 | 426 | 14 | 435 | 36 | ++ | |
| 158 | 10 | 169 | 414 | 421 | 9 | 432 | 36 | ++ | + |
| 158 | 10 | 169 | 414 | 424 | 12 | 433 | 36 | ++ | + |
| 158 | 10 | 169 | 414 | 426 | 14 | 435 | 36 | + | + |
| 160 | 12 | 171 | 414 | 421 | 9 | 432 | 36 | ++ | |
| 160 | 12 | 171 | 414 | 424 | 12 | 433 | 36 | ++ | + |
| 160 | 12 | 171 | 414 | 426 | 14 | 435 | 36 | + | + |
| 156 | 8 | 168 | 441 | 449 | 10 | 459 | 57 | + | + |
| 156 | 8 | 168 | 441 | 451 | 12 | 460 | 57 | + | + |
| 156 | 8 | 168 | 441 | 453 | 14 | 462 | 57 | + | + |
| 158 | 10 | 169 | 441 | 449 | 10 | 459 | 57 | + | + |
| 158 | 10 | 169 | 441 | 451 | 12 | 460 | 57 | + | + |
| 158 | 10 | 169 | 441 | 453 | 14 | 462 | 57 | + | + |
| 160 | 12 | 171 | 441 | 449 | 10 | 459 | 57 | + | + |
| 160 | 12 | 171 | 441 | 451 | 12 | 460 | 57 | + | + |
| 160 | 12 | 171 | 441 | 453 | 14 | 462 | 57 | + | + |
| 156 | 8 | 168 | 468 | 476 | 10 | 486 | 111 | +++ | + |
| 156 | 8 | 168 | 468 | 478 | 12 | 487 | 111 | +++ | + |
| 156 | 8 | 168 | 468 | 480 | 14 | 489 | 111 | +++ | + |
| 158 | 10 | 169 | 468 | 476 | 10 | 486 | 111 | ++ | + |
| 158 | 10 | 169 | 468 | 478 | 12 | 487 | 111 | +++ | + |
| 158 | 10 | 169 | 468 | 480 | 14 | 489 | 111 | ++ | + |
| 160 | 12 | 171 | 468 | 476 | 10 | 486 | 111 | ++ | + |
| 160 | 12 | 171 | 468 | 478 | 12 | 487 | 111 | ++ | + |
| 160 | 12 | 171 | 468 | 480 | 14 | 489 | 111 | ++ | + |
| 156 | 8 | 168 | 495 | 503 | 10 | 513 | 56 | ++ | + |
| 156 | 8 | 168 | 495 | 505 | 12 | 514 | 56 | ++ | + |
| 156 | 8 | 168 | 495 | 508 | 15 | 516 | 56 | +++ | + |
| 158 | 10 | 169 | 495 | 503 | 10 | 513 | 56 | + | + |
| 158 | 10 | 169 | 495 | 505 | 12 | 514 | 56 | ++ | + |
| 158 | 10 | 169 | 495 | 508 | 15 | 516 | 56 | ++ | + |
| 160 | 12 | 171 | 495 | 503 | 10 | 513 | 56 | + | + |
| 160 | 12 | 171 | 495 | 505 | 12 | 514 | 56 | ++ | + |
| 160 | 12 | 171 | 495 | 508 | 15 | 516 | 56 | ++ | + |
| 156 | 8 | 168 | 522 | 529 | 9 | 547 | 80 | + | + |
| 156 | 8 | 168 | 522 | 531 | 11 | 550 | 80 | ++ | + |
| 156 | 8 | 168 | 522 | 533 | 13 | 554 | 80 | ++ | + |
| 158 | 10 | 169 | 522 | 529 | 9 | 547 | 80 | + | + |
| 158 | 10 | 169 | 522 | 531 | 11 | 550 | 80 | ++ | + |
| 158 | 10 | 169 | 522 | 533 | 13 | 554 | 80 | + | + |
| 160 | 12 | 171 | 522 | 529 | 9 | 547 | 80 | + | + |
| 160 | 12 | 171 | 522 | 531 | 11 | 550 | 80 | ++ | + |
| 160 | 12 | 171 | 522 | 533 | 13 | 554 | 80 | + | + |
| 156 | 8 | 168 | 566 | 573 | 9 | 584 | 41 | + | + |
| 156 | 8 | 168 | 566 | 575 | 11 | 585 | 41 | + | + |
| 156 | 8 | 168 | 566 | 577 | 13 | 587 | 41 | + | + |
| 158 | 10 | 169 | 566 | 573 | 9 | 584 | 41 | + | + |
| 158 | 10 | 169 | 566 | 575 | 11 | 585 | 41 | + | + |
| 158 | 10 | 169 | 566 | 577 | 13 | 587 | 41 | + | + |
| 160 | 12 | 171 | 566 | 573 | 9 | 584 | 41 | + | + |
| 160 | 12 | 171 | 566 | 575 | 11 | 585 | 41 | + | + |
| 160 | 12 | 171 | 566 | 577 | 13 | 587 | 41 | + | + |

### Example 3—Dual Prime Editing of the TRAC Gene in HEK 293T Cells

Based on results of the first screen in Example 2, 5′ PEgRNAs containing the spacer sequence of SEQ ID NO: 4 or SEQ ID NO: 88 were further tested in pairs with 3′ PEgRNAs containing the spacer sequence of SEQ ID NO: 368, 522, or 177. For each 5′ spacer and 3′ spacer, additional PBS lengths and RTT lengths were examined compared to PERNAs tested in Example 2. Like Example 2, the RTT sequences were designed to replace the endogenous IND sequence in TRAC with the 38 bp attB sequence recognizable by Bxb1. However, in this Example, for each spacer-PBS combination, two RTT sequences were tested: one corresponding to the full length 38nt attB sequence (or reverse complement thereof), the other corresponding to a 30nt sequence at the 3′ end of the 38nt sequence. Accordingly, the 5′ PEgRNAs contained an RTT sequence according to SEQ ID NO: 24 or SEQ ID NO: 27, and the 3′ PEgRNAs contained an RTT sequence according to SEQ ID NO: 106 or SEQ ID NO: 107. In this experiment, PEgRNA pairs having the same length of RTTs were tested, so the RTT pairs are either 100% complementary to each other throughout their entire length, or share 22 bp complementarity between each other. All of the PERNAs contained a gRNA core sequence according to SEQ ID NO: 590.

The PEgRNAs were assembled from U6 expression plasmids, and HEK293T cells were transfected with PEgRNA pairs and plasmid encoding a prime editor fusion protein as described in Example 1. 72 hours after transfection, genomic DNA was extracted by aspirating media, adding 100 μL of QuickExtract DNA Extraction solution (Lucigen, #QR9050) and incubating according to manufacturer's protocol for high-throughput sequencing. 192 PERNA pairs were successfully tested, with highest editing efficiency reaching above 60% (Tables 32A-32B). Two technical replicates were included for each PEgRNA pairs. Sequence numbers of the PEgRNAs and PEgRNA components, as well as editing efficiency and indel frequency are summarized in Tables 32A-32B. As transcribed, the PEgRNAs in Tables 32A and 32B will contain, from 5′ to 3′, the indicated PEgRNA sequence and a variable number of 3′ Us (e.g., up to 4 Us). The editing efficiency and indel frequency scales in Tables 32A and 32B are as follows. Editing efficiency: 11%-24%: +; 24%-37%: ++; 37%-50%: +++; 50%-63%: ++++. Indel frequency: 0.5%-4%: +; 4%-8%: ++; 7%-12%: +++; 12%-16%: ++++.

TABLE 32A
Dual prime editing efficiency in HEK293T cells. All 5′ PEgRNAs have a spacer sequence according to SEQ ID NO: 4.

| 5′ | | | | | 3′ | 3′ | | | | | nick- | | | | |
|---|---|---|---|---|---|---|---|---|---|---|---|---|---|---|---|
| RTT | | | | 5′ | Spacer | RTT | | | | 3′ | to- | | | | |
| SEQ | 5′ | 5′ PBS | 5′ | PEgRNA | SEQ | SEQ | 3′ | 3′ PBS | 3′ | PEgRNA | nick | % | % | % | % |
| ID | RTT | sequence | PBS | SEQ | ID | ID | RTT | sequence | PBS | SEQ | dis- | edit_ | edit_ | indel_ | indel_ |
| NO | length | number | length | ID NO | NO | NO | length | number | length | ID NO | tance | rep1 | rep2 | rep1 | rep2 |
| 24 | 30 | 13 | 11 | 32 | 177 | 106 | 30 | 184 | 9 | 203 | 74 | +++ | ++++ | ++ | ++ |
| 27 | 38 | 13 | 11 | 44 | 177 | 107 | 38 | 184 | 9 | 222 | 74 | ++++ | ++++ | + | + |
| 24 | 30 | 15 | 13 | 34 | 177 | 106 | 30 | 184 | 9 | 203 | 74 | +++ | ++++ | + | ++ |
| 27 | 38 | 15 | 13 | 49 | 177 | 107 | 38 | 184 | 9 | 222 | 74 | ++++ | ++++ | + | + |
| 24 | 30 | 17 | 15 | 38 | 177 | 106 | 30 | 184 | 9 | 203 | 74 | +++ | +++ | + | ++ |
| 27 | 38 | 17 | 15 | 52 | 177 | 107 | 38 | 184 | 9 | 222 | 74 | +++ | +++ | + | + |
| 24 | 30 | 19 | 17 | 42 | 177 | 106 | 30 | 184 | 9 | 203 | 74 | ++ | ++ | + | + |
| 27 | 38 | 19 | 17 | 54 | 177 | 107 | 38 | 184 | 9 | 222 | 74 | ++ | ++ | + | + |
| 24 | 30 | 13 | 11 | 32 | 177 | 106 | 30 | 186 | 11 | 212 | 74 | +++ | +++ | ++ | ++ |
| 27 | 38 | 13 | 11 | 44 | 177 | 107 | 38 | 186 | 11 | 224 | 74 | ++++ | ++++ | + | + |
| 24 | 30 | 15 | 13 | 34 | 177 | 106 | 30 | 186 | 11 | 212 | 74 | +++ | +++ | ++ | ++ |
| 27 | 38 | 15 | 13 | 49 | 177 | 107 | 38 | 186 | 11 | 224 | 74 | ++++ | ++++ | + | + |
| 24 | 30 | 17 | 15 | 38 | 177 | 106 | 30 | 186 | 11 | 212 | 74 | ++ | ++ | ++ | + |
| 27 | 38 | 17 | 15 | 52 | 177 | 107 | 38 | 186 | 11 | 224 | 74 | +++ | +++ | + | + |
| 24 | 30 | 19 | 17 | 42 | 177 | 106 | 30 | 186 | 11 | 212 | 74 | ++ | ++ | + | + |
| 27 | 38 | 19 | 17 | 54 | 177 | 107 | 38 | 186 | 11 | 224 | 74 | + | ++ | + | + |
| 24 | 30 | 13 | 11 | 32 | 177 | 106 | 30 | 188 | 13 | 215 | 74 | +++ | +++ | ++ | ++ |
| 27 | 38 | 13 | 11 | 44 | 177 | 107 | 38 | 188 | 13 | 226 | 74 | ++++ | ++++ | + | + |
| 24 | 30 | 15 | 13 | 34 | 177 | 106 | 30 | 188 | 13 | 215 | 74 | +++ | +++ | ++ | ++ |
| 27 | 38 | 15 | 13 | 49 | 177 | 107 | 38 | 188 | 13 | 226 | 74 | ++++ | ++++ | + | + |
| 24 | 30 | 17 | 15 | 38 | 177 | 106 | 30 | 188 | 13 | 215 | 74 | ++ | ++ | + | ++ |
| 27 | 38 | 17 | 15 | 52 | 177 | 107 | 38 | 188 | 13 | 226 | 74 | +++ | +++ | + | + |
| 24 | 30 | 19 | 17 | 42 | 177 | 106 | 30 | 188 | 13 | 215 | 74 | + | + | + | + |
| 27 | 38 | 19 | 17 | 54 | 177 | 107 | 38 | 188 | 13 | 226 | 74 | + | ++ | + | + |
| 24 | 30 | 13 | 11 | 32 | 177 | 106 | 30 | 190 | 15 | 219 | 74 | +++ | +++ | + | ++ |
| 27 | 38 | 13 | 11 | 44 | 177 | 107 | 38 | 190 | 15 | 228 | 74 | ++++ | ++++ | + | + |
| 24 | 30 | 15 | 13 | 34 | 177 | 106 | 30 | 190 | 15 | 219 | 74 | +++ | +++ | + | ++ |
| 27 | 38 | 15 | 13 | 49 | 177 | 107 | 38 | 190 | 15 | 228 | 74 | ++++ | ++++ | + | + |
| 24 | 30 | 17 | 15 | 38 | 177 | 106 | 30 | 190 | 15 | 219 | 74 | ++ | ++ | + | + |
| 27 | 38 | 17 | 15 | 52 | 177 | 107 | 38 | 190 | 15 | 228 | 74 | +++ | +++ | + | + |
| 24 | 30 | 19 | 17 | 42 | 177 | 106 | 30 | 190 | 15 | 219 | 74 | ++ | ++ | + | + |
| 27 | 38 | 19 | 17 | 54 | 177 | 107 | 38 | 190 | 15 | 228 | 74 | + | + | + | + |
| 24 | 30 | 13 | 11 | 32 | 368 | 106 | 30 | 373 | 7 | 390 | 63 | +++ | +++ | ++ | ++ |
| 27 | 38 | 13 | 11 | 44 | 368 | 107 | 38 | 373 | 7 | 401 | 63 | ++++ | ++++ | + | + |
| 24 | 30 | 15 | 13 | 34 | 368 | 106 | 30 | 373 | 7 | 390 | 63 | +++ | +++ | ++ | ++ |
| 27 | 38 | 15 | 13 | 49 | 368 | 107 | 38 | 373 | 7 | 401 | 63 | ++++ | ++++ | + | + |
| 24 | 30 | 17 | 15 | 38 | 368 | 106 | 30 | 373 | 7 | 390 | 63 | +++ | ++ | ++ | ++ |
| 27 | 38 | 17 | 15 | 52 | 368 | 107 | 38 | 373 | 7 | 401 | 63 | +++ | +++ | + | + |
| 24 | 30 | 19 | 17 | 42 | 368 | 106 | 30 | 373 | 7 | 390 | 63 | ++ | ++ | + | + |
| 27 | 38 | 19 | 17 | 54 | 368 | 107 | 38 | 373 | 7 | 401 | 63 | ++ | ++ | + | + |
| 24 | 30 | 13 | 11 | 32 | 368 | 106 | 30 | 375 | 9 | 392 | 63 | +++ | +++ | ++ | ++ |
| 27 | 38 | 13 | 11 | 44 | 368 | 107 | 38 | 375 | 9 | 405 | 63 | ++++ | ++++ | + | + |
| 24 | 30 | 15 | 13 | 34 | 368 | 106 | 30 | 375 | 9 | 392 | 63 | +++ | +++ | ++ | ++ |
| 27 | 38 | 15 | 13 | 49 | 368 | 107 | 38 | 375 | 9 | 405 | 63 | ++++ | ++++ | + | + |
| 24 | 30 | 17 | 15 | 38 | 368 | 106 | 30 | 375 | 9 | 392 | 63 | ++ | ++ | ++ | ++ |
| 27 | 38 | 17 | 15 | 52 | 368 | 107 | 38 | 375 | 9 | 405 | 63 | +++ | +++ | + | + |
| 24 | 30 | 19 | 17 | 42 | 368 | 106 | 30 | 375 | 9 | 392 | 63 | ++ | ++ | + | + |
| 27 | 38 | 19 | 17 | 54 | 368 | 107 | 38 | 375 | 9 | 405 | 63 | ++ | ++ | + | + |
| 24 | 30 | 13 | 11 | 32 | 368 | 106 | 30 | 377 | 11 | 395 | 63 | +++ | +++ | ++ | ++ |
| 27 | 38 | 13 | 11 | 44 | 368 | 107 | 38 | 377 | 1 | 408 | 63 | ++++ | ++++ | + | + |
| 24 | 30 | 15 | 13 | 34 | 368 | 106 | 30 | 377 | 1 | 395 | 63 | +++ | +++ | ++ | ++ |
| 27 | 38 | 15 | 13 | 49 | 368 | 107 | 38 | 377 | 11 | 408 | 63 | ++++ | ++++ | + | + |
| 24 | 30 | 17 | 15 | 38 | 368 | 106 | 30 | 377 | 11 | 395 | 63 | ++ | ++ | ++ | + |
| 27 | 38 | 17 | 15 | 52 | 368 | 107 | 38 | 377 | 11 | 408 | 63 | +++ | +++ | + | + |
| 24 | 30 | 19 | 17 | 42 | 368 | 106 | 30 | 377 | 11 | 395 | 63 | ++ | ++ | + | + |
| 27 | 38 | 19 | 17 | 54 | 368 | 107 | 38 | 377 | 11 | 408 | 63 | + | ++ | + | + |
| 24 | 30 | 13 | 11 | 32 | 368 | 106 | 30 | 379 | 13 | 398 | 63 | ++ | ++ | ++++ | ++++ |
| 27 | 38 | 13 | 11 | 44 | 368 | 107 | 38 | 379 | 13 | 410 | 63 | ++++ | ++++ | + | + |
| 24 | 30 | 15 | 13 | 34 | 368 | 106 | 30 | 379 | 13 | 398 | 63 | ++ | ++ | ++++ | +++ |
| 27 | 38 | 15 | 13 | 49 | 368 | 107 | 38 | 379 | 13 | 410 | 63 | ++++ | ++++ | + | + |
| 24 | 30 | 17 | 15 | 38 | 368 | 106 | 30 | 379 | 13 | 398 | 63 | ++ | + | +++ | +++ |
| 27 | 38 | 17 | 15 | 52 | 368 | 107 | 38 | 379 | 13 | 410 | 63 | +++ | +++ | + | + |
| 24 | 30 | 19 | 17 | 42 | 368 | 106 | 30 | 379 | 13 | 398 | 63 | + | + | +++ | ++ |
| 27 | 38 | 19 | 17 | 54 | 368 | 107 | 38 | 379 | 13 | 410 | 63 | + | + | + | + |
| 24 | 30 | 13 | 11 | 32 | 522 | 106 | 30 | 531 | 11 | 543 | 70 | +++ | +++ | ++ | ++ |
| 27 | 38 | 13 | 11 | 44 | 522 | 107 | 38 | 531 | 11 | 556 | 70 | ++++ | ++++ | + | + |
| 24 | 30 | 15 | 13 | 34 | 522 | 106 | 30 | 531 | 11 | 543 | 70 | +++ | +++ | + | ++ |
| 27 | 38 | 15 | 13 | 49 | 522 | 107 | 38 | 531 | 11 | 556 | 70 | ++++ | ++++ | + | + |
| 24 | 30 | 17 | 15 | 38 | 522 | 106 | 30 | 531 | 11 | 543 | 70 | +++ | ++ | + | + |
| 27 | 38 | 17 | 15 | 52 | 522 | 107 | 38 | 531 | 11 | 556 | 70 | +++ | +++ | + | + |
| 24 | 30 | 19 | 17 | 42 | 522 | 106 | 30 | 531 | 11 | 543 | 70 | ++ | ++ | + | + |
| 27 | 38 | 19 | 17 | 54 | 522 | 107 | 38 | 531 | 1 | 556 | 70 | + | ++ | + | + |
| 24 | 30 | 13 | 11 | 32 | 522 | 106 | 30 | 533 | 13 | 545 | 70 | ++++ | +++ | ++ | ++ |
| 27 | 38 | 13 | 11 | 44 | 522 | 107 | 38 | 533 | 13 | 558 | 70 | ++++ | ++++ | + | + |
| 24 | 30 | 15 | 13 | 34 | 522 | 106 | 30 | 533 | 13 | 545 | 70 | +++ | +++ | ++ | ++ |
| 27 | 38 | 15 | 13 | 49 | 522 | 107 | 38 | 533 | 13 | 558 | 70 | ++++ | ++++ | + | + |
| 24 | 30 | 17 | 15 | 38 | 522 | 106 | 30 | 533 | 13 | 545 | 70 | +++ | ++ | ++ | + |
| 27 | 38 | 17 | 15 | 52 | 522 | 107 | 38 | 533 | 13 | 558 | 70 | +++ | +++ | + | + |
| 24 | 30 | 19 | 17 | 42 | 522 | 106 | 30 | 533 | 13 | 545 | 70 | ++ | ++ | + | + |
| 27 | 38 | 19 | 17 | 54 | 522 | 107 | 38 | 533 | 13 | 558 | 70 | ++ | ++ | + | + |
| 24 | 30 | 13 | 11 | 32 | 522 | 106 | 30 | 535 | 15 | 549 | 70 | +++ | +++ | + | ++ |
| 27 | 38 | 13 | 11 | 44 | 522 | 107 | 38 | 535 | 15 | 561 | 70 | ++++ | ++++ | + | + |
| 24 | 30 | 15 | 13 | 34 | 522 | 106 | 30 | 535 | 15 | 549 | 70 | +++ | +++ | + | ++ |
| 27 | 38 | 15 | 13 | 49 | 522 | 107 | 38 | 535 | 15 | 561 | 70 | ++++ | +++ | + | + |
| 24 | 30 | 17 | 15 | 38 | 522 | 106 | 30 | 535 | 15 | 549 | 70 | ++ | ++ | + | + |
| 27 | 38 | 17 | 15 | 52 | 522 | 107 | 38 | 535 | 15 | 561 | 70 | +++ | +++ | + | + |
| 24 | 30 | 19 | 17 | 42 | 522 | 106 | 30 | 535 | 15 | 549 | 70 | ++ | ++ | + | + |
| 27 | 38 | 19 | 17 | 54 | 522 | 107 | 38 | 535 | 15 | 561 | 70 | + | + | + | + |
| 24 | 30 | 13 | 11 | 32 | 522 | 106 | 30 | 537 | 17 | 552 | 70 | +++ | +++ | + | ++ |
| 27 | 38 | 13 | 11 | 44 | 522 | 107 | 38 | 537 | 17 | 562 | 70 | +++ | +++ | + | + |
| 24 | 30 | 15 | 13 | 34 | 522 | 106 | 30 | 537 | 17 | 552 | 70 | +++ | +++ | + | ++ |
| 27 | 38 | 15 | 13 | 49 | 522 | 107 | 38 | 537 | 17 | 562 | 70 | +++ | +++ | + | + |
| 24 | 30 | 17 | 15 | 38 | 522 | 106 | 30 | 537 | 17 | 552 | 70 | ++ | ++ | + | + |
| 27 | 38 | 17 | 15 | 52 | 522 | 107 | 38 | 537 | 17 | 562 | 70 | ++ | ++ | + | + |
| 24 | 30 | 19 | 17 | 42 | 522 | 106 | 30 | 537 | 17 | 552 | 70 | + | + | + | + |
| 27 | 38 | 19 | 17 | 54 | 522 | 107 | 38 | 537 | 17 | 562 | 70 | + | + | + | + |

TABLE 32B
Dual prime editing efficiency in HEK293T cells. All 5′ PEgRNAs have a spacer sequence according to SEQ ID NO: 88.

| 5′ | | | | | 3′ | 3′ | | | | | nick- | | | | |
|---|---|---|---|---|---|---|---|---|---|---|---|---|---|---|---|
| RTT | | | | 5′ | Spacer | RTT | | | | 3′ | to- | | | | |
| SEQ | 5′ | 5′ PBS | 5′ | PEgRNA | SEQ | SEQ | 3′ | 3′ PBS | 3′ | PEgRNA | nick | % | % | % | % |
| ID | RTT | sequence | PBS | SEQ | ID | ID | RTT | sequence | PBS | SEQ | dis- | edit_ | edit_ | indel_ | indel_ |
| NO | length | number | length | ID NO | NO | NO | length | number | length | ID NO | tance | rep1 | rep2 | rep1 | rep2 |
| 24 | 30 | 97 | 11 | 111 | 177 | 106 | 30 | 184 | 9 | 203 | 83 | +++ | ++++ | ++ | ++ |
| 27 | 38 | 97 | 11 | 129 | 177 | 107 | 38 | 184 | 9 | 222 | 83 | ++++ | ++++ | + | + |
| 24 | 30 | 99 | 13 | 114 | 177 | 106 | 30 | 184 | 9 | 203 | 83 | +++ | +++ | + | + |
| 27 | 38 | 99 | 13 | 134 | 177 | 107 | 38 | 184 | 9 | 222 | 83 | ++++ | ++++ | + | + |
| 24 | 30 | 101 | 15 | 120 | 177 | 106 | 30 | 184 | 9 | 203 | 83 | +++ | +++ | + | ++ |
| 27 | 38 | 101 | 15 | 140 | 177 | 107 | 38 | 184 | 9 | 222 | 83 | ++++ | ++++ | + | + |
| 24 | 30 | 103 | 17 | 125 | 177 | 106 | 30 | 184 | 9 | 203 | 83 | ++ | ++ | + | + |
| 27 | 38 | 103 | 17 | 143 | 177 | 107 | 38 | 184 | 9 | 222 | 83 | +++ | +++ | + | + |
| 24 | 30 | 97 | 11 | 111 | 177 | 106 | 30 | 186 | 11 | 212 | 83 | +++ | ++++ | ++ | ++ |
| 27 | 38 | 97 | 11 | 129 | 177 | 107 | 38 | 186 | 11 | 224 | 83 | ++++ | ++++ | + | + |
| 24 | 30 | 99 | 13 | 114 | 177 | 106 | 30 | 186 | 11 | 212 | 83 | +++ | +++ | + | + |
| 27 | 38 | 99 | 13 | 134 | 177 | 107 | 38 | 186 | 11 | 224 | 83 | +++ | ++++ | + | + |
| 24 | 30 | 101 | 15 | 120 | 177 | 106 | 30 | 186 | 11 | 212 | 83 | +++ | +++ | + | + |
| 27 | 38 | 101 | 15 | 140 | 177 | 107 | 38 | 186 | 11 | 224 | 83 | ++++ | ++++ | + | + |
| 24 | 30 | 103 | 17 | 125 | 177 | 106 | 30 | 186 | 11 | 212 | 83 | ++ | ++ | + | + |
| 27 | 38 | 103 | 17 | 143 | 177 | 107 | 38 | 186 | 11 | 224 | 83 | +++ | +++ | + | + |
| 24 | 30 | 97 | 11 | 111 | 177 | 106 | 30 | 188 | 13 | 215 | 83 | +++ | +++ | ++ | ++ |
| 27 | 38 | 97 | 11 | 129 | 177 | 107 | 38 | 188 | 13 | 226 | 83 | ++++ | ++++ | + | + |
| 24 | 30 | 99 | 13 | 114 | 177 | 106 | 30 | 188 | 13 | 215 | 83 | ++ | +++ | + | + |
| 27 | 38 | 99 | 13 | 134 | 177 | 107 | 38 | 188 | 13 | 226 | 83 | +++ | ++++ | + | + |
| 24 | 30 | 101 | 15 | 120 | 177 | 106 | 30 | 188 | 13 | 215 | 83 | ++ | +++ | + | + |
| 27 | 38 | 101 | 15 | 140 | 177 | 107 | 38 | 188 | 13 | 226 | 83 | +++ | ++++ | + | + |
| 24 | 30 | 103 | 17 | 125 | 177 | 106 | 30 | 188 | 13 | 215 | 83 | + | ++ | + | + |
| 27 | 38 | 103 | 17 | 143 | 177 | 107 | 38 | 188 | 13 | 226 | 83 | +++ | +++ | + | + |
| 24 | 30 | 97 | 11 | 111 | 177 | 106 | 30 | 190 | 15 | 219 | 83 | +++ | +++ | + | + |
| 27 | 38 | 97 | 11 | 129 | 177 | 107 | 38 | 190 | 15 | 228 | 83 | ++++ | ++++ | + | + |
| 24 | 30 | 99 | 13 | 114 | 177 | 106 | 30 | 190 | 15 | 219 | 83 | +++ | +++ | + | + |
| 27 | 38 | 99 | 13 | 134 | 177 | 107 | 38 | 190 | 15 | 228 | 83 | +++ | +++ | + | + |
| 24 | 30 | 101 | 15 | 120 | 177 | 106 | 30 | 190 | 15 | 219 | 83 | +++ | +++ | + | + |
| 27 | 38 | 101 | 15 | 140 | 177 | 107 | 38 | 190 | 15 | 228 | 83 | +++ | +++ | + | + |
| 24 | 30 | 103 | 17 | 125 | 177 | 106 | 30 | 190 | 15 | 219 | 83 | ++ | ++ | + | + |
| 27 | 38 | 103 | 17 | 143 | 177 | 107 | 38 | 190 | 15 | 228 | 83 | +++ | ++ | + | + |
| 24 | 30 | 97 | 11 | 111 | 368 | 106 | 30 | 373 | 7 | 390 | 72 | ++++ | +++ | ++ | ++ |
| 27 | 38 | 97 | 11 | 129 | 368 | 107 | 38 | 373 | 7 | 401 | 72 | ++++ | ++++ | + | + |
| 24 | 30 | 99 | 13 | 114 | 368 | 106 | 30 | 373 | 7 | 390 | 72 | +++ | +++ | ++ | + |
| 27 | 38 | 99 | 13 | 134 | 368 | 107 | 38 | 373 | 7 | 401 | 72 | +++ | ++++ | + | + |
| 24 | 30 | 101 | 15 | 120 | 368 | 106 | 30 | 373 | 7 | 390 | 72 | +++ | ++ | ++ | + |
| 27 | 38 | 101 | 15 | 140 | 368 | 107 | 38 | 373 | 7 | 401 | 72 | ++++ | ++++ | + | + |
| 24 | 30 | 103 | 17 | 125 | 368 | 106 | 30 | 373 | 7 | 390 | 72 | ++ | ++ | + | + |
| 27 | 38 | 103 | 17 | 143 | 368 | 107 | 38 | 373 | 7 | 401 | 72 | +++ | +++ | + | + |
| 24 | 30 | 97 | 11 | 111 | 368 | 106 | 30 | 375 | 9 | 392 | 72 | +++ | +++ | ++ | ++ |
| 27 | 38 | 97 | 11 | 129 | 368 | 107 | 38 | 375 | 9 | 405 | 72 | ++++ | ++++ | + | + |
| 24 | 30 | 99 | 13 | 114 | 368 | 106 | 30 | 375 | 9 | 392 | 72 | +++ | +++ | ++ | ++ |
| 27 | 38 | 99 | 13 | 134 | 368 | 107 | 38 | 375 | 9 | 405 | 72 | ++++ | ++++ | + | + |
| 24 | 30 | 101 | 15 | 120 | 368 | 106 | 30 | 375 | 9 | 392 | 72 | +++ | ++ | ++ | ++ |
| 27 | 38 | 101 | 15 | 140 | 368 | 107 | 38 | 375 | 9 | 405 | 72 | +++ | +++ | + | + |
| 24 | 30 | 103 | 17 | 125 | 368 | 106 | 30 | 375 | 9 | 392 | 72 | ++ | ++ | + | + |
| 27 | 38 | 103 | 17 | 143 | 368 | 107 | 38 | 375 | 9 | 405 | 72 | +++ | +++ | + | + |
| 24 | 30 | 97 | 11 | 111 | 368 | 106 | 30 | 377 | 11 | 395 | 72 | +++ | +++ | ++ | ++ |
| 27 | 38 | 97 | 11 | 129 | 368 | 107 | 38 | 377 | 11 | 408 | 72 | ++++ | ++++ | + | + |
| 24 | 30 | 99 | 13 | 114 | 368 | 106 | 30 | 377 | 11 | 395 | 72 | +++ | ++ | ++ | + |
| 27 | 38 | 99 | 13 | 134 | 368 | 107 | 38 | 377 | 11 | 408 | 72 | +++ | ++++ | + | + |
| 24 | 30 | 101 | 15 | 120 | 368 | 106 | 30 | 377 | 11 | 395 | 72 | +++ | ++ | ++ | + |
| 27 | 38 | 101 | 15 | 140 | 368 | 107 | 38 | 377 | 11 | 408 | 72 | ++++ | ++++ | + | + |
| 24 | 30 | 103 | 17 | 125 | 368 | 106 | 30 | 377 | 11 | 395 | 72 | ++ | + | + | + |
| 27 | 38 | 103 | 17 | 143 | 368 | 107 | 38 | 377 | 11 | 408 | 72 | +++ | +++ | + | + |
| 24 | 30 | 97 | 11 | 111 | 368 | 106 | 30 | 379 | 13 | 398 | 72 | +++ | ++ | ++++ | ++++ |
| 27 | 38 | 97 | 11 | 129 | 368 | 107 | 38 | 379 | 13 | 410 | 72 | ++++ | ++++ | + | + |
| 24 | 30 | 99 | 13 | 114 | 368 | 106 | 30 | 379 | 13 | 398 | 72 | ++ | + | +++ | +++ |
| 27 | 38 | 99 | 13 | 134 | 368 | 107 | 38 | 379 | 13 | 410 | 72 | +++ | +++ | + | + |
| 24 | 30 | 101 | 15 | 120 | 368 | 106 | 30 | 379 | 13 | 398 | 72 | ++ | ++ | +++ | +++ |
| 27 | 38 | 101 | 15 | 140 | 368 | 107 | 38 | 379 | 13 | 410 | 72 | +++ | ++++ | + | + |
| 24 | 30 | 103 | 17 | 125 | 368 | 106 | 30 | 379 | 13 | 398 | 72 | + | + | ++ | ++ |
| 27 | 38 | 103 | 17 | 143 | 368 | 107 | 38 | 379 | 13 | 410 | 72 | ++ | +++ | + | + |
| 24 | 30 | 97 | 11 | 111 | 522 | 106 | 30 | 531 | 11 | 543 | 79 | ++++ | +++ | ++ | ++ |
| 27 | 38 | 97 | 11 | 129 | 522 | 107 | 38 | 531 | 11 | 556 | 79 | ++++ | ++++ | + | + |
| 24 | 30 | 99 | 13 | 114 | 522 | 106 | 30 | 531 | 11 | 543 | 79 | +++ | +++ | + | + |
| 27 | 38 | 99 | 13 | 134 | 522 | 107 | 38 | 531 | 11 | 556 | 79 | ++++ | ++++ | + | + |
| 24 | 30 | 101 | 15 | 120 | 522 | 106 | 30 | 531 | 11 | 543 | 79 | +++ | +++ | + | + |
| 27 | 38 | 101 | 15 | 140 | 522 | 107 | 38 | 531 | 11 | 556 | 79 | ++++ | ++++ | + | + |
| 24 | 30 | 103 | 17 | 125 | 522 | 106 | 30 | 531 | 11 | 543 | 79 | ++ | ++ | + | + |
| 27 | 38 | 103 | 17 | 143 | 522 | 107 | 38 | 531 | 11 | 556 | 79 | +++ | +++ | + | + |
| 24 | 30 | 97 | 11 | 111 | 522 | 106 | 30 | 533 | 13 | 545 | 79 | ++++ | ++++ | ++ | ++ |
| 27 | 38 | 97 | 11 | 129 | 522 | 107 | 38 | 533 | 13 | 558 | 79 | ++++ | ++++ | + | + |
| 24 | 30 | 99 | 13 | 114 | 522 | 106 | 30 | 533 | 13 | 545 | 79 | +++ | +++ | ++ | + |
| 27 | 38 | 99 | 13 | 134 | 522 | 107 | 38 | 533 | 13 | 558 | 79 | ++++ | ++++ | + | + |
| 24 | 30 | 101 | 15 | 120 | 522 | 106 | 30 | 533 | 13 | 545 | 79 | +++ | +++ | + | + |
| 27 | 38 | 101 | 15 | 140 | 522 | 107 | 38 | 533 | 13 | 558 | 79 | +++ | ++++ | + | + |
| 24 | 30 | 103 | 17 | 125 | 522 | 106 | 30 | 533 | 13 | 545 | 79 | ++ | ++ | + | + |
| 27 | 38 | 103 | 17 | 143 | 522 | 107 | 38 | 533 | 13 | 558 | 79 | +++ | +++ | + | + |
| 24 | 30 | 97 | 11 | 111 | 522 | 106 | 30 | 535 | 15 | 549 | 79 | +++ | +++ | + | ++ |
| 27 | 38 | 97 | 11 | 129 | 522 | 107 | 38 | 535 | 15 | 561 | 79 | ++++ | ++++ | + | + |
| 24 | 30 | 99 | 13 | 114 | 522 | 106 | 30 | 535 | 15 | 549 | 79 | +++ | +++ | + | + |
| 27 | 38 | 99 | 13 | 134 | 522 | 107 | 38 | 535 | 15 | 561 | 79 | +++ | +++ | + | + |
| 24 | 30 | 101 | 15 | 120 | 522 | 106 | 30 | 535 | 15 | 549 | 79 | +++ | +++ | + | ++ |
| 27 | 38 | 101 | 15 | 140 | 522 | 107 | 38 | 535 | 15 | 561 | 79 | +++ | +++ | + | + |
| 24 | 30 | 103 | 17 | 125 | 522 | 106 | 30 | 535 | 15 | 549 | 79 | ++ | ++ | + | + |
| 27 | 38 | 103 | 17 | 143 | 522 | 107 | 38 | 535 | 15 | 561 | 79 | ++ | ++ | + | + |
| 24 | 30 | 97 | 11 | 111 | 522 | 106 | 30 | 537 | 17 | 552 | 79 | +++ | +++ | + | + |
| 27 | 38 | 97 | 11 | 129 | 522 | 107 | 38 | 537 | 17 | 562 | 79 | +++ | +++ | + | + |
| 24 | 30 | 99 | 13 | 114 | 522 | 106 | 30 | 537 | 17 | 552 | 79 | ++ | +++ | + | + |
| 27 | 38 | 99 | 13 | 134 | 522 | 107 | 38 | 537 | 17 | 562 | 79 | ++ | ++ | + | + |
| 24 | 30 | 101 | 15 | 120 | 522 | 106 | 30 | 537 | 17 | 552 | 79 | ++ | +++ | + | + |
| 27 | 38 | 101 | 15 | 140 | 522 | 107 | 38 | 537 | 17 | 562 | 79 | +++ | +++ | + | + |
| 24 | 30 | 103 | 17 | 125 | 522 | 106 | 30 | 537 | 17 | 552 | 79 | + | ++ | + | + |
| 27 | 38 | 103 | 17 | 143 | 522 | 107 | 38 | 537 | 17 | 562 | 79 | + | ++ | + | + |

### Example 4—Dual Prime Editing of the TRAC Gene in HEK 293T Cells

To test dual prime editing efficiency with alternative RTTs, PEgRNA pairs were designed to replace the IND in TRAC with a 38 bp attB sequence into the TRAC locus in a reversed orientation compared to the insert demonstrated in Examples 2 and 3. 5′ PEgRNAs containing the spacer sequence according to SEQ ID NO: 88 and 3′ PEgRNAs containing the spacer sequence according to SEQ ID NO: 522 or 177 were further tested with the altered RTT sequences. For each 5′ spacer-PBS and 3′ spacer-PBS combination, two sets of RTT pairs were tested: a first set containing a 5′ RTT and a 3′ RTT, and a second by switching the 5′ RTT and 3′ RTT of the first set. This experiment thus tested PEgRNAs designed to replace the TRAC IND with a 38 bp attB sequence, where the 38 bp attB insert is in both orientations. For each pair of PEgRNAs, RTTs of equal length were used. For example, for a 5′ spacer according to SEQ ID NO: 88 combined with a 11nt PBS and a 3′ spacer according to SEQ ID NO: 522 combined with a 13nt PBS, two pairs of PEgRNAs were tested: the first pair contained a 5′ RTT according to SEQ ID NO: 24 and a 3′ RTT according to SEQ ID NO: 106, and the second pair contained a 5′ RTT according to SEQ ID NO: 106 and a 3′ RTT according to SEQ ID NO: 24. The 5′ PERNAs contained PBSs of 11 or 13nt, and all 3′ PERNAs contained 13nt PBSs. All of the PEgRNAs contained a gRNA core sequence according to SEQ ID NO: 590.

The PEgRNAs were assembled from U6 expression plasmids, and HEK293T cells were transfected with PEgRNA pairs and plasmid encoding a prime editor fusion protein as described in Example 1. 72 hours after transfection, genomic DNA was extracted by aspirating media, adding 100 μL of QuickExtract DNA Extraction solution (Lucigen, #QR9050) and incubating according to manufacturer's protocol for high-throughput sequencing. 192 PEgRNA pairs were successfully tested, with highest editing efficiency reaching above 60% (Table 33). Two technical replicates were included for each PEgRNA pair.

Successful editing was observed with PEgRNA pairs containing RTTs that encode the attB insert sequence in both orientations. Sequence numbers of the PEgRNAs and PEgRNA components, as well as editing efficiency and indels are summarized in Table 33. As transcribed, the PEgRNAs in Table 33 will contain, from 5′ to 3′, the indicated PEgRNA sequence and a variable number of 3′ Us (e.g., up to 4 Us). The editing efficiency and indel frequency scales in Table 33 are as follows. Editing efficiency: 29%-39%: +; 39%-48%: ++; 48%-57%: +++; 57%-67%: ++++. Indel frequency: 1.6%-2.4%: +; 2.4%-4%: ++; 4%-6%: +++; 6%-8%: ++++.

TABLE 33
Dual prime editing efficiency in HEK293T cells. All 5′ PEgRNAs have a spacer sequence according to SEQ ID NO: 88

| 5′ | | | | | 3′ | 3′ | | | | | nick- | | | | |
|---|---|---|---|---|---|---|---|---|---|---|---|---|---|---|---|
| RTT | | | | 5′ | Spacer | RTT | | | | 3′ | to- | | | | |
| SEQ | 5′ | 5′ PBS | 5′ | PEgRNA | SEQ | SEQ | 3′ | 3′ PBS | 3′ | PEgRNA | nick | % | % | % | % |
| ID | RTT | sequence | PBS | SEQ | ID | ID | RTT | sequence | PBS | SEQ | dis- | edit_ | edit_ | indel_ | indel_ |
| NO | length | number | length | ID NO | NO | NO | length | number | length | ID NO | tance | rep1 | rep2 | rep1 | rep2 |
| 24 | 30 | 97 | 11 | 111 | 522 | 106 | 30 | 533 | 13 | 541 | 79 | ++++ | ++++ | +++ | +++ |
| 106 | 30 | 97 | 11 | 112 | 522 | 24 | 30 | 533 | 13 | 542 | 79 | ++ | + | ++++ | ++++ |
| 24 | 30 | 97 | 11 | 111 | 177 | 106 | 30 | 188 | 13 | 210 | 83 | +++ | +++ | ++++ | ++++ |
| 106 | 30 | 97 | 11 | 112 | 177 | 24 | 30 | 188 | 13 | 211 | 83 | +++ | +++ | ++++ | ++++ |
| 27 | 38 | 97 | 11 | 129 | 522 | 107 | 38 | 533 | 13 | 554 | 79 | ++++ | ++++ | ++ | ++ |
| 107 | 38 | 97 | 11 | 130 | 522 | 27 | 38 | 533 | 13 | 555 | 79 | ++ | ++ | ++ | ++ |
| 27 | 38 | 97 | 11 | 129 | 177 | 107 | 38 | 188 | 13 | 221 | 83 | ++++ | ++++ | ++ | ++ |
| 107 | 38 | 97 | 11 | 130 | 177 | 27 | 38 | 188 | 13 | 591 | 83 | ++++ | ++++ | ++ | ++ |
| 24 | 30 | 99 | 13 | 114 | 522 | 106 | 30 | 533 | 13 | 541 | 79 | +++ | ++ | +++ | +++ |
| 106 | 30 | 99 | 13 | 115 | 522 | 24 | 30 | 533 | 13 | 542 | 79 | + | + | +++ | +++ |
| 24 | 30 | 99 | 13 | 114 | 177 | 106 | 30 | 188 | 13 | 210 | 83 | ++ | ++ | +++ | ++++ |
| 106 | 30 | 99 | 13 | 115 | 177 | 24 | 30 | 188 | 13 | 211 | 83 | ++ | ++ | ++++ | +++ |
| 27 | 38 | 99 | 13 | 134 | 522 | 107 | 38 | 533 | 13 | 554 | 79 | ++++ | +++ | + | + |
| 107 | 38 | 99 | 13 | 135 | 522 | 27 | 38 | 533 | 13 | 555 | 79 | + | + | + | + |
| 27 | 38 | 99 | 13 | 134 | 177 | 107 | 38 | 188 | 13 | 221 | 83 | ++++ | ++++ | + | + |
| 107 | 38 | 99 | 13 | 135 | 177 | 27 | 38 | 188 | 13 | 591 | 83 | ++ | ++ | + | + |

### Example 5—Dual Prime Editing for Introducing a Pa01 Recombinase Recognition Site in the TRAC Gene in HEK 293T Cells

To test dual prime editing efficiency with additional RTTs, PEgRNA pairs were designed to replace the IND with a 39 bp attB sequence or a 33 bp attB sequence, both of which are substrates of recombinase Pa01. The 33 bp and the 39 bp sequences, and the corresponding RTT sequences encoding the inserts, are referred to as Pa01_1 and Pa01_2, respectively. RTT pairs having full complementarity or partial complementarity to each other were designed: for Pa01_1, a pair of 33nt RTTs are fully complementary to each other, and a second pair of 27nt RTTs share 21 bp of complementarity. For Pa01_2, a pair of 39nt RTTs are fully complementary to each other, and a second pair of 30nt RTTs share 21 bp of complementarity. The 5′ PEgRNAs in this experiment have a spacer sequence according to SEQ ID NO: 4 or 88, and the 3′ PEgRNAs have a sequence according to SEQ ID NO: 177. All PEgNAs in this experiment have a PBS of 13 nucleotides in length.

The PEgRNAs were assembled from U6 expression plasmids, and HEK293T cells were transfected with PEgRNA pairs and plasmid encoding a prime editor fusion protein as described in Example 1. 72 hours after transfection, genomic DNA was extracted by aspirating media, adding 100 μL of QuickExtract DNA Extraction solution (Lucigen, #QR9050) and incubating according to manufacturer's protocol for high-throughput sequencing.

7 PEgRNA pairs were successfully tested, with two technical replicates included for each pair of PEgRNAs. At least 10% and up to above 50% editing efficiency was observed (Table 34). In addition, comparable editing efficiencies were observed in PEgRNA pairs having fully complementary and partially complementary RTTs.

As transcribed, the PEgRNAs in Table 34 will contain, from 5′ to 3′, the indicated PEgRNA sequence and a variable number of 3′ Us (e.g., up to 4 Us). The editing efficiency and indel frequency scales in Table 34 are as follows. Editing efficiency: 9%-20%: +; 20%-31%: ++; 31%-42%: +++; 42%-54%: ++++. Indel frequency: 0.4%-1%: +; 1%-1.5%: ++; 1.5%-2.1%: +++; 2.1%-2.7%: ++++. The PEgRNA pairs in rows 2-5 (italicized) contained Pa01_2 RTTs, and the other PEgRNA pairs contained Pa01_1 RTTs.

TABLE 34
Dual prime editing efficiency in HEK293T cells.

| 5′ | 5′ | | | 3′ | 3′ | | | | | | |
|---|---|---|---|---|---|---|---|---|---|---|---|
| Spacer | RTT | | 5′ | Spacer | RTT | 3′ | 3′ | | | | |
| SEQ | SEQ | 5′ PBS | PEgRN | SEQ | SEQ | PBS | pegRNA | % | % | % | % |
| ID | ID | sequence | A SEQ | ID | ID | sequence | SEQ ID | edit_ | edit_ | indel_ | indel_ |
| NO | NO | number | ID NO | NO | NO | number | NO | rep1 | rep2 | rep1 | rep2 |
| 88 | 25 | 99 | 592 | 177 | 197 | 188 | 595 | + | + | + | + |
| 88 | 28 | 99 | 593 | 177 | 199 | 188 | 597 | + | + | + | + |
| 4 | 28 | 15 | 50 | 177 | 199 | 188 | 597 | +++ | +++ | + | + |
| 4 | 25 | 15 | 33 | 177 | 197 | 188 | 595 | ++++ | ++++ | ++++ | ++++ |
| 88 | 22 | 99 | 594 | 177 | 195 | 188 | 599 | + | + | ++ | ++ |
| 4 | 22 | 15 | 31 | 177 | 195 | 188 | 599 | ++++ | ++++ | ++++ | ++++ |
| 4 | 26 | 15 | 39 | 177 | 198 | 188 | 601 | ++++ | ++++ | +++ | +++ |

### Example 6—Dual Prime Editing for Introducing a Bxb1 Recombinase Recognition in T Cells

The ability of dual prime editing to introduce recombinase recognition site in human T cells was examined. Based on editing efficiency in HEK293T cells, 5′ PEgRNAs having a spacer sequence according to SEQ ID NO: 4 or 88 and 3′ PEgRNAs having a spacer sequence according to SEQ ID NO: 177 or 368 were tested in this experiment. The PEgRNAs contain RTT sequences for replacement of the IND with the 38 bp Bxb1-attB sequence, with the 5′ RTT and 3′ RTT pairs either fully complementary to each other or sharing 20 base pairs of complementarity.

8 PegRNA pairs were tested in human T cells. The cells were electroporated with polynucleotide encoding a prime editor fusion protein and PEgRNA pairs, and genomic DNA was extracted as described in Example 1. Sequence analysis was performed with amplicon sequencing using primers designed to target edited sequences. Except for the PEgRNA pair according to SEQ ID NO: 109 and SEQ ID NO: 388, two technical replicates were included for each pair. The results are summarized in Table 35.

All PEgRNAs used in Table 35 experimentally contained a 3′ UUUU end adaptation, and further contained chemical modifications 3′ mN*mN*mN*N and 5′mN*mN*mN*N modifications, where m indicates that the nucleotide contains a 2′-O-Me modification and a * indicates a phosphorothioate bond. The editing efficiency and indel frequency scales in Table 35 are as follows. Editing efficiency: 12%-23%: +; 23%-35%: ++; 35%-47%: +++; 47%-58%: ++++. Indel frequency: 2%-6%: +; 6%-11%: ++; 11%-15%: +++; 15%-20%: ++++.

TABLE 35
Dual prime editing efficiency in human T cells.

| 5′ | 5′ | | | | 5′ | 3′ | 3′ | | | | 3′ | nick- | | | | |
|---|---|---|---|---|---|---|---|---|---|---|---|---|---|---|---|---|
| spacer | RTT | | 5′ | | PERNA | spacer | RTT | | 3′ | | PEgRNA | to- | % | % | % | % |
| SEQ | SEQ | 5′ | PBS | 5′ | SEQ | SEQ | SEQ | 3′ | PBS | 3′ | SEQ | nick | edit_ | edit_ | indel_ | indel_ |
| ID | ID | RTT | sequence | PBS | ID | ID | ID | RTT | sequence | PBS | ID | dis- | rep1 | rep2 | rep1 | rep2 |
| NO | NO | length | number | length | NO | NO | NO | length | number | length | NO | tance | | | | |
| 4 | 27 | 38 | 13 | 11 | 44 | 177 | 107 | 38 | 188 | 13 | 221 | 74 | +++ | +++ | ++ | + |
| 4 | 27 | 38 | 13 | 11 | 44 | 368 | 107 | 38 | 376 | 10 | 400 | 63 | + | + | + | + |
| 88 | 27 | 38 | 96 | 10 | 126 | 177 | 107 | 38 | 188 | 13 | 221 | 83 | ++++ | ++++ | ++ | ++ |
| 88 | 27 | 38 | 96 | 10 | 126 | 368 | 107 | 38 | 376 | 10 | 400 | 72 | + | + | + | + |
| 4 | 23 | 29 | 13 | 11 | 30 | 177 | 196 | 29 | 188 | 13 | 207 | 74 | ++++ | ++++ | +++ | +++ |
| 4 | 23 | 29 | 13 | 11 | 30 | 368 | 196 | 29 | 376 | 10 | 388 | 63 | ++ | ++ | ++ | +++ |
| 88 | 23 | 29 | 96 | 10 | 109 | 177 | 196 | 29 | 188 | 13 | 207 | 83 | ++++ | ++++ | +++ | ++++ |
| 88 | 23 | 29 | 96 | 10 | 109 | 368 | 196 | 29 | 376 | 10 | 388 | 72 | | ++ | | +++ |

### Example 7—Dual Prime Editing for Disruption of TRAC Expression and Integration of CD19 CAR

An exemplary pair of PEgRNAs (5′ PEgRNA: SEQ ID NO: 126; 3′ PERNA: SEQ ID NO: 221) from Example 6 where successful dual prime editing was observed were examined for dual prime editing mediated donor integration in the presence of a recombinase. Human T cells were electroporated with the PEgRNA pair, mRNA encoding a prime editor fusion protein, mRNA encoding the recombinase Bxb1, and a DNA donor sequence encoding a 3.5 kb cargo of CD19 CAR, RQR8 epitope marker, and GFP separated by 2A self-cleaving peptide sequences.

The T cells were electroporated with the PEgRNA pair, mRNA encoding a prime editor fusion protein, Bxb1 mRNA, and DNA donor as described in Example 1. The experiment included a non-transfection control where the cells were only electroporated with electroporation buffer, and a no-Bxb1 control where the T cells were only contacted with the prime editor mRNA, PEgRNAs and the DNA donor but not the Bxb1 mRNA. Three different doses were tested for both the DNA donor and Bxb1 mRNA. For prime editing efficiency, ddPCR primers were designed to target the unedited endogenous TRAC sequence, attB sequence encoded by the RTTs at TRAC, and attL sequence expected to result from Bxb1 mediated integration at TRAC. Accordingly, ddPCR quantification of the unedited TRAC sequence, attB sequence, and attL sequence would reflect the abundance of cells containing unedited, dual prime edited (but no Bxb1 mediated integration), and successful recombination at the TRAC gene. After electroporation, genomic DNA was extracted for ddPCR analysis. For ddPCR, editing efficiency was assessed using copy number/μL of the target sequences (TRAC, attB, attL) compared to copy number/μL of a reference gene RPP30 (Biorad).

CD3 protein loss and transgene expression after electroporation were evaluated via flow cytometry detection of CD3, GFP, and CD19-CAR. The following antibodies, proteins, and dyes were used: phycoerythrin (PE)-conjugated anti-CD3 (clone SK7; BD Biosciences), Atto 647N-conjugated recombinant human CD19 (BioTechne), SYTOX Blue dead cell stain (ThermoFisher).

The efficiency of dual prime editing, CD3 protein loss, and CD19 CAR integration are summarized in Table 36.

TABLE 36
Dual prime editing mediated donor integration in TRAC in human T cells. All PEgRNAs used
experimentally contained a 3′ UUUU end adaptation, and further contained chemical
modifications 3′ mN*mN*mN*N and 5′mN*mN*mN*N modifications, where m indicates that
the nucleotide contains a 2′-O-Me modification and a * indicates a phosphorothioate bond.

| 5′ | 3′ | | | % CD3 | | % CD19- | % unedited | | |
|---|---|---|---|---|---|---|---|---|---|
| PEgRNA | PERNA | Bxb1 | DNA | protein | % GFP | CAR | TRAC | % attB | % attL |
| SEQ ID | SEQ ID | mRNA | donor | loss | integration | integration | (ddPCR) | (ddPCR) | (ddPCR) |
| non- | non- | 0 | 0 | 0.00 | 0.01 | 0.69 | 102.14 | 0.04 | 0.07 |
| electro- | electro- | | | | | | | | |
| poration | poration | | | | | | | | |
| control | control | | | | | | | | |
| 126 | 221 | 0 (no-Bxb1 | dose 3 | 89.47 | 1.08 | 0.65 | 8.40 | 84.51 | 0.09 |
| | | control) | | | | | | | |
| 126 | 221 | dose 1 | dose 1 | 92.23 | 38.00 | 36.70 | 7.28 | 52.55 | 27.92 |
| 126 | 221 | dose 1 | dose 2 | 93.23 | 61.10 | 58.70 | 4.35 | 39.41 | 51.10 |
| 126 | 221 | dose 1 | dose 3 | 92.39 | 60.10 | 58.60 | 5.84 | 35.27 | 55.38 |
| 126 | 221 | dose 2 | dose 1 | 91.25 | 35.10 | 34.40 | 6.77 | 56.47 | 31.39 |
| 126 | 221 | dose 2 | dose 2 | 90.99 | 36.40 | 35.00 | 6.02 | 40.64 | 46.06 |
| 126 | 221 | dose 2 | dose 3 | 92.22 | 53.50 | 51.80 | 12.78 | 38.37 | 44.32 |
| 126 | 221 | dose 3 | dose 1 | 78.11 | 47.80 | 46.70 | 7.30 | 54.00 | 32.19 |
| 126 | 221 | dose 3 | dose 2 | 92.27 | 49.70 | 48.10 | 5.18 | 41.16 | 43.52 |
| 126 | 221 | dose 3 | dose 3 | 45.38 | 24.80 | 24.80 | 33.29 | 25.15 | 34.38 |

### Example 8—Multiplexed Prime Editing in Human T Cells

The ability of multiplexed editing using dual prime editing and single PEgRNA editing was examined. A pair of dual prime editing PEgRNAs were designed to insert a 38 bp attB sequence at target gene CCR5 or AAVS1, respectively. A single PEgRNA and a nicking guide RNA (ngRNA) were also designed to convert a C to T in the VEGFA gene. Human T cells were cultured and electroporated with polynucleotide encoding a prime editor fusion protein and chemically synthesized PEgRNAs, and editing efficiency was examined as described in Example 1. For single-plex editing, the T cells were electroporated with PEgRNAs (or PEgRNA and ngRNA for VEGFA) that target one gene. For multiplex editing, the T cells were electroporated with PEgRNAs and ngRNA that target all three genes.

Sequences of the PEgRNAs and ngRNA, as well as the editing efficiency, are provided in Table 37. All PEgRNAs and ngRNA used experimentally in Table 37 contained a 3′ UUUU end adaptation, and further contained chemical modifications 3′ mN*mN*mN*N and 5′mN*mN*mN*N modifications, where m indicates that the nucleotide contains a 2′-O-Me modification and a * indicates a phosphorothioate bond. The editing efficiency and indel frequency scales in Table 37 are as follows. Editing efficiency: 23%-35%: +; 35%-46%: ++; 46%-58%: +++; 58%-69%: ++++. Indel frequency: 8%-9%: +; 9%-10%: ++; 10%-11%: +++; 11%-12%: ++++.

TABLE 37
Multiplexed and single-plexed prime editing in human T cells. For VEGFA, the PEgRNA and ngRNA
used are shown.

| | | | | | | | % | % | % | % |
|---|---|---|---|---|---|---|---|---|---|---|
| | | | | | | | indels | indels | editing | editing |
| Gene | 5′ | | | 3′ | | | single- | multi- | single- | multi- |
| target | Spacer | 5′ RTT | 5′ PBS | Spacer | 3′ RTT | 3′ PBS | plex | xple | plex | plex |
| CCR5 | AAGTG | ATGAT | CCCAA | GTATG | GGCTT | GCTCTC | ++++ | ++ | +++ | +++ |
| | TGATC | CCTGA | GTGAT | GAAAA | GTCGA | ATT | | | | |
| | ACTTG | CGACG | C (SEQ | TGAGA | CGACG | (SEQ ID | | | | |
| | GGTGG | GAGAC | ID NO: | GCTGC | GCGGT | NO: | | | | |
| | (SEQ ID | CGCCG | 1222) | (SEQ ID | CTCCGT | 1224) | | | | |
| | NO: | TCGTCG | | NO: | CGTCA | | | | | |
| | 1221) | ACAAG | | 1223) | GGATC | | | | | |
| | | CC (SEQ | | | AT (SEQ | | | | | |
| | | ID NO: | | | ID NO: | | | | | |
| | | 27 | | | 107) | | | | | |
| AAVS1 | GACGT | ATGAT | GGCAG | GCGAC | GGCTT | CCACTT | + | + | ++++ | ++++ |
| | CACGG | CCTGA | CGCC | TCCTGG | GTCGA | CCAGG | | | | |
| | CGCTG | CGACG | (SEQ ID | AAGTG | CGACG | (SEQ ID | | | | |
| | CCCCA | GAGAC | NO: | GCCA | GCGGT | NO: | | | | |
| | (SEQ ID | CGCCG | 1226) | (SEQ ID | CTCCGT | 1228) | | | | |
| | NO: | TCGTCG | | NO: | CGTCA | | | | | |
| | 1225) | ACAAG | | 1227) | GGATC | | | | | |
| | | CC (SEQ | | | AT (SEQ | | | | | |
| | | ID NO: | | | ID NO: | | | | | |
| | | 27 | | | 107) | | | | | |
| VEGFA | GATGT | AATGT | TCTGGC | GATGT | NA | NA | + | +++ | + | + |
| | CTGCA | GCCAT | CTGCA | ACAGA | | | | | | |
| | GGCCA | CTGGA | GA (SEQ | GAGCC | | | | | | |
| | GATGA | GCACT | ID NO: | CAGGG | | | | | | |
| | (SEQ ID | CA (SEQ | 1231) | C (SEQ | | | | | | |
| | NO: | ID NO: | | ID NO: | | | | | | |
| | 1229) | 1230) | | 1232) | | | | | | |

### Example 9—Dual Prime Editing of the TRAC Gene in Human T Cells

5′ PEgRNAs containing the spacer sequence of SEQ ID NO: 4 or SEQ ID NO: 88 were tested in pairs with 3′ PEgRNAs containing the spacer sequence of SEQ ID NOs: 368, 522, or 177 for editing of the TRAC gene in human T cells. The RTT sequences were designed to replace the endogenous IND sequence in TRAC with an attB sequence. The 5′ PEgRNAs contained an RTT sequence according to SEQ ID NO: 27, and the 3′ PEgRNAs contained an RTT sequence according to SEQ ID NOs: 27 or 107. For 3′ TRAC PEgRNA with SEQ ID NO: 1127 and 5′ TRAC PERNA SEQ ID NO: 136, both PERNAs included RTT sequence according to SEQ ID NO: 27. Likewise, for 3′ TRAC PERNA with SEQ ID NO: 1128 and 5′ TRAC PERNA SEQ ID NO: 136, both PERNAs included RTT sequence according to SEQ ID NO: 27. All of the PEgRNAs contained a gRNA core sequence according to SEQ ID NO: 590. All PEgRNAs were chemically synthesized and contained 3′ UUUU end adaptation, and further contained chemical modifications 3′ mN*mN*mN*N and 5′mN*mN*mN*N modifications, where m indicates that the nucleotide contains a 2′-O-Me modification and a * indicates a phosphorothioate bond.mRNA encoding a prime editor having a Cas9 H840A nickase and a MMLV-RT pentamutant (D200N,T330P,L603W,T306K,W313F) was generated via in vitro transcription.

T cells were cultured in ImmunoCult XF T cell Expansion medium (StemCell Technologies) containing 1% L-glutamine, 1% Penicillin/Streptomycin (Lonza), and 100 IU/mL IL-2 (PeproTech). T cells were activated with DynaBeads Human T-Activator CD3/CD28 (ThermoFisher) at a 1:1 bead: cell ratio for 48 hours prior to electroporation.

After 48 hours of stimulation, DynaBeads were magnetically removed, and T cells were washed once with PBS prior to resuspension in electroporation buffer. T cells were resuspended in MaxCyte electroporation buffer containing Prime Editor mRNA, and TRAC 5′ pegRNA, TRAC 3′ pegRNA and electroporated using MaxCyte. Following electroporation, T cells were transferred to an empty well-plate and allowed to recover at 37° C., 5% CO₂ for 20 minutes. Subsequently, cells were suspended in basal medium (without cytokines) and incubated at 37° C., 5% CO₂ for 20 minutes before being suspended in complete T cell medium. Genomic DNA was extracted using Lucigen QuickExtraction Solution according to manufacturer's protocol for sequence analysis.

All experiments were performed as n=2 biological replicates using CD3+ T cells from two healthy human donors. Unedited T cells and mock-edited cells (electroporation only) were included as negative controls. Dual Prime editing efficiency of attB integration and indels frequency were assessed by amplicon sequencing using the primers listed in Table 38 for amplification of the TRAC locus. The primers bear Illumina forward and reverse adapters, allowing amplicons generating in a first round of PCR (PCR1) to serve as templates for a second round of PCR (PCR2) with unique Illumina barcoding primer pairs. PCR2 products were pooled by common amplicons, purified by gel electrophoresis, and quantified by Qubit (ThermoFisher). Libraries were sequenced on an Illumina MiSeq instrument using Illumina MiSeq control software. Reads were demultiplexed by the Illumina MiSeq control software and analyzed using CRISPResso 2.

CD3 protein expression was evaluated via flow cytometry detection of CD3, using phycoerythrin (PE)-conjugated anti-CD3 (clone SK7; BD Biosciences). For dual Prime edited cells, CD3 protein loss was assessed by normalizing to average CD3 expression of mock (electroporation only) controls. Gating schema of the follow cytometry excluded dead cells to avoid auto-fluorescent signals. In addition, SYTOX Blue dead cell stain (ThermoFisher) was used to detect cell death. No appreciable cell death or appreciable difference in cell death between edited and negative controls were detected in the flow cytometry.

TABLE 38
Primers for detecting TRAC editing

| Primer | |
|---|---|
| name | Sequence |
| TRAC | ACACTCTTTCCCTACACGACGCTCTTCCGATCTNNNNATG |
| Forward | AGACCGTGACTTGCCAG (SEQ ID NO: 1233) |
| TRAC | TGGAGTTCAGACGTGTGCTCTTCCGATCTTGCTGTTGTTG |
| Reverse | AAGGCGTTT (SEQID NO: 1234) |

Sequence numbers of the PEgRNAs and PEgRNA components, as well as indel frequency, percent attB insertion, and percent CD3 protein loss are summarized in Table 39. “-”: not applicable.

TABLE 39
Dual prime editing efficiency of TRAC in human T cells

| 5′ TRAC | 5′ spacer | 5′ PBS | 5′ RTT | 3′ TRAC | 3′ spacer | 3′ PBS | 3′ RTT | TRAC | TRAC | CD3 |
|---|---|---|---|---|---|---|---|---|---|---|
| PEgRNA | SEQ | SEQ | SEQ | PEgRNA | SEQ | SEQ | SEQ | attB | indels | protein |
| SEQ ID | ID | ID | ID | SEQ ID | ID | ID | ID | % | % | loss % |
| 136 | 88 | 96 | 27 | 226 | 177 | 188 | 107 | 58.22 | 20.47 | 77.40 |
| 136 | 88 | 96 | 27 | 403 | 368 | 374 | 107 | 61.63 | 16.36 | 76.36 |
| 136 | 88 | 96 | 27 | 558 | 522 | 533 | 107 | 59.17 | 13.64 | 68.07 |
| 136 | 88 | 96 | 27 | 224 | 177 | 186 | 107 | 71.89 | 13.86 | 85.09 |
| 136 | 88 | 96 | 27 | 1127 | 177 | 184 | 27 | 1.63 | 81.89 | 90.22 |
| 136 | 88 | 96 | 27 | 1128 | 368 | 373 | 27 | 11.26 | 69.78 | 92.85 |
| 136 | 88 | 96 | 27 | 556 | 522 | 531 | 107 | 71.91 | 15.60 | 88.61 |
| 51 | 4 | 14 | 27 | 226 | 177 | 188 | 107 | 57.75 | 14.78 | 69.67 |
| 51 | 4 | 14 | 27 | 403 | 368 | 374 | 107 | 58.90 | 13.24 | 72.14 |
| 51 | 4 | 14 | 27 | 558 | 522 | 533 | 107 | 63.79 | 9.20 | 70.69 |
| 51 | 4 | 14 | 27 | 224 | 177 | 186 | 107 | 63.26 | 10.76 | 75.35 |
| 51 | 4 | 14 | 27 | 1127 | 177 | 184 | 27 | 1.73 | 76.29 | 89.55 |
| 51 | 4 | 14 | 27 | 1128 | 368 | 373 | 27 | 11.29 | 53.55 | 86.63 |
| 51 | 4 | 14 | 27 | 556 | 522 | 531 | 107 | 66.08 | 10.06 | 76.76 |
| 141 | 88 | 98 | 27 | 226 | 177 | 188 | 107 | 48.88 | 19.34 | 63.36 |
| 141 | 88 | 98 | 27 | 403 | 368 | 374 | 107 | 62.18 | 19.83 | 83.18 |
| 141 | 88 | 98 | 27 | 558 | 522 | 533 | 107 | 56.42 | 17.83 | 73.60 |
| 141 | 88 | 98 | 27 | 224 | 177 | 186 | 107 | 63.37 | 18.94 | 82.28 |
| 141 | 88 | 98 | 27 | 1127 | 177 | 184 | 27 | 1.64 | 65.59 | 76.41 |
| 141 | 88 | 98 | 27 | 1128 | 368 | 373 | 27 | 6.51 | 63.51 | 86.49 |
| 141 | 88 | 98 | 27 | 556 | 522 | 531 | 107 | 64.86 | 18.95 | 84.97 |
| 53 | 4 | 15 | 27 | 226 | 177 | 188 | 107 | 38.29 | 12.01 | 43.05 |
| 53 | 4 | 15 | 27 | 403 | 368 | 374 | 107 | 55.15 | 17.65 | 74.60 |
| 53 | 4 | 15 | 27 | 558 | 522 | 533 | 107 | 51.22 | 10.36 | 62.05 |
| 53 | 4 | 15 | 27 | 224 | 177 | 186 | 107 | 56.84 | 13.08 | 74.05 |
| 53 | 4 | 15 | 27 | 1127 | 177 | 184 | 27 | 1.36 | 36.22 | 48.26 |
| 53 | 4 | 15 | 27 | 1128 | 368 | 373 | 27 | 4.35 | 38.51 | 55.68 |
| 53 | 4 | 15 | 27 | 556 | 522 | 531 | 107 | 61.09 | 13.00 | 76.46 |
| Unedited | — | — | — | Unedited | — | — | — | 0 | 0.025 | — |
| Unedited | — | — | — | Unedited | — | — | — | 0.01 | 0.01 | — |
| Unedited | — | — | — | Unedited | — | — | — | 0 | 0.05 | — |
| Unedited | — | — | — | Unedited | — | — | — | 0.01 | 0.07 | — |
| Unedited | — | — | — | Unedited | — | — | — | 0.01 | 0.05 | — |
| Mock | — | — | — | Mock | — | — | — | 0 | 0.02 | — |
| Mock | — | — | — | Mock | — | — | — | 0 | 0 | — |
| Mock | — | — | — | Mock | — | — | — | 0.02 | 0.02 | — |
| Mock | — | — | — | Mock | — | — | — | 0.01 | 0.05 | — |
| Mock | — | — | — | Mock | — | — | — | 0 | 0.05 | — |

### Example 10—Dual Prime Editing of the TRAC Gene in Human T Cells

5′ PEgRNAs containing the spacer sequence of SEQ ID NO: 4 or SEQ ID NO: 88 were tested in pairs with 3′ PEgRNAs containing the spacer sequence of SEQ ID NOs: 177, 368, or 522 for editing of the TRAC gene in human T cells. The RTT sequences were designed to replace the endogenous IND sequence in TRAC with an attB sequence. The 5′ PEgRNAs contained an RTT sequence according to SEQ ID NO: 24, and the 3′ PEgRNAs contained an RTT sequence according to SEQ ID NO: 24 or 196. For a few PEgRNA “pairs”, both the 5′ PEgRNA and the 3′ PEgRNA contained the same RTT sequence (in other words, while the 5′ PEgRNA spacer targets the upstream sequence and the 3′ PEgRNA spacer targets the downstream sequence of an editing target sequence, reverse complementarity between the 5′ RTT and the 3′ RTT is not expected): for 3′ TRAC PEgRNA with SEQ ID NOs: 1135-1138, or 546 and 5′ TRAC PEgRNA SEQ ID NO: 118, both PEgRNAs included RTT sequence according to SEQ ID NO: 24. Likewise, for 3′ TRAC PEgRNA with SEQ ID NO: 1138 and 5′ TRAC PEgRNA SEQ ID NO: 36, both PEgRNAs included RTT sequence according to SEQ ID NO: 24. All of the PEgRNAs contained a gRNA core sequence according to SEQ ID NO: 590. All PEgRNAs were chemically synthesized and contained 3′ UUUU end adaptation, and further contained chemical modifications 3′ mN*mN*mN*N and 5′mN*mN*mN*N modifications, where m indicates that the nucleotide contains a 2′-O-Me modification and a * indicates a phosphorothioate bond. mRNA encoding a prime editor having a Cas9 H840A nickase and a MMLV-RT pentamutant (D200N,T330P,L603W,T306K,W313F) was generated via in vitro transcription. For PEgRNA pairs having 5′ PEgRNA as set forth in SEQ ID NO: 1132 and 3′ PEgRNAs as set forth in SEQ ID NO: 1139, 1140, or 1141, the length of the reverse complementarity (the “OD” length) between the 5′ RTT and the 3′ RTT of these PERNA pairs was 20 base pairs (bp), and the length of insertion (the “RD” length) was 38 bp. For all other PEgRNA pairs where the 5′ RTT and 3′ RTT are reverse complements to each other at the 5′ ends, the length of the reverse complementarity (the “OD” length) between the 5′ RTT and the 3′ RTT of these PERNA pairs was 21 base pairs (bp), and the length of insertion (the “RD” length) was 38 bp.

T cells were cultured, activated and electroporated, and genomic DNA was extracted as described in Example 9.

All experiments were performed as n=2 biological replicates using CD3+ T cells from two healthy human donors. Unedited T cells and mock-edited cells (electroporation only) were included as negative controls. Dual Prime editing efficiency of attB integration and indels frequency were assessed by amplicon sequencing using the primers listed in Table 38 for amplification of the TRAC locus. The primers bear Illumina forward and reverse adapters, allowing amplicons generating in a first round of PCR (PCR1) to serve as templates for a second round of PCR (PCR2) with unique Illumina barcoding primer pairs. PCR2 products were pooled by common amplicons, purified by gel electrophoresis, and quantified by Qubit (ThermoFisher). Libraries were sequenced on an Illumina MiSeq instrument using Illumina MiSeq control software. Reads were demultiplexed by the Illumina MiSeq control software and analyzed using CRISPResso 2.

CD3 protein expression was evaluated via flow cytometry detection of CD3, using phycoerythrin (PE)-conjugated anti-CD3 (clone SK7; BD Biosciences). For dual Prime edited cells, CD3 protein loss was assessed by normalizing to average CD3 expression of mock (electroporation only) controls. Gating schema of the follow cytometry excluded dead cells to avoid auto-fluorescent signals. In addition, SYTOX Blue dead cell stain (ThermoFisher) was used to detect cell death. No appreciable cell death or appreciable difference in cell death between edited and negative controls were detected in the flow cytometry. The forward and reverse primers used to detect TRAC editing are shown in Table 38.

Sequence numbers of the PEgRNAs and PERNA components, as well as indel frequency, percent attB insertion, and percent CD3 protein loss are summarized in Table 40. “-”: not applicable.

TABLE 40
Dual prime editing efficiency of TRAC in human T cells

| 5′ TRAC | 5′ spacer | 5′ PBS | 5′ RTT | 3′ TRAC | 3′ spacer | 3′ PBS | 3′ RTT | TRAC | TRAC | CD3 |
|---|---|---|---|---|---|---|---|---|---|---|
| PEgRNA | SEQ | SEQ | SEQ | PEgRNA | SEQ | SEQ | SEQ | attB | indels | protein |
| SEQ ID | ID | ID | ID | SEQ ID | ID | ID | ID | % | % | loss % |
| Unedited | — | — | — | Unedited | — | — | — | 0.01 | 0.00 | 0.00 |
| Mock | — | — | — | Mock | — | — | — | 0.00 | 0.01 | 0.71 |
| 118 | 88 | 97 | 24 | 1135 | 522 | 531 | 24 | 27.07 | 32.18 | 56.97 |
| 118 | 88 | 97 | 24 | 546 | 522 | 533 | 24 | 16.45 | 24.78 | 34.40 |
| 118 | 88 | 97 | 24 | 1136 | 177 | 186 | 24 | 22.87 | 60.37 | 89.19 |
| 118 | 88 | 97 | 24 | 1137 | 368 | 373 | 24 | 49.20 | 32.88 | 91.58 |
| 118 | 88 | 97 | 24 | 1138 | 368 | 375 | 24 | 48.85 | 32.05 | 92.43 |
| 118 | 88 | 97 | 24 | 1139 | 177 | 188 | 196 | 77.83 | 13.73 | 94.85 |
| 118 | 88 | 97 | 24 | 1140 | 368 | 374 | 196 | 79.11 | 11.08 | 95.51 |
| 118 | 88 | 97 | 24 | 1141 | 177 | 186 | 196 | 79.60 | 13.12 | 95.95 |
| 118 | 88 | 97 | 24 | 1142 | 522 | 533 | 196 | 81.05 | 11.87 | 96.66 |
| 123 | 88 | 99 | 24 | 1139 | 177 | 188 | 196 | 58.49 | 30.37 | 93.72 |
| 123 | 88 | 99 | 24 | 1140 | 368 | 374 | 196 | 55.39 | 29.49 | 89.11 |
| 123 | 88 | 99 | 24 | 1141 | 177 | 186 | 196 | 55.15 | 28.76 | 87.09 |
| 36 | 4 | 13 | 24 | 1138 | 368 | 375 | 24 | 6.51 | 68.27 | 84.37 |
| 36 | 4 | 13 | 24 | 1139 | 177 | 188 | 196 | 61.71 | 25.76 | 89.55 |
| 36 | 4 | 13 | 24 | 1140 | 368 | 374 | 196 | 59.70 | 25.36 | 90.66 |
| 36 | 4 | 13 | 24 | 1141 | 177 | 186 | 196 | 62.61 | 26.54 | 92.88 |
| 1132 | 88 | 96 | 23 | 1139 | 177 | 188 | 196 | 61.61 | 26.67 | 91.39 |
| 1132 | 88 | 96 | 23 | 1140 | 368 | 374 | 196 | 64.06 | 25.52 | 92.72 |
| 1132 | 88 | 96 | 23 | 1141 | 177 | 186 | 196 | 64.45 | 26.19 | 93.35 |

### Example 11—Multiplexed Prime Editing of TRAC and B2M in Human T Cells and Integration of CD19 CAR

The ability of multiplexed editing using dual prime editing of a TRAC locus and single PEgRNA editing of B2M was examined. 5′ PEgRNAs having the sequences of SEQ ID NO:s 136 and 51 and 3′ PEgRNAs having the sequences of SEQ ID NOs: 224 and 558 were tested for targeted integration of a 38 bp attB sequence GGCTTGTCGACGACGGCGGTCTCCGTCGTCAGGATCAT (SEQ ID NO: 1187) at the TRAC locus. The prime editor mRNA was assembled as described in Example 9. To test attB-attP-Bxb1 mediated integration of donor sequence, mRNA encoding a wildtype Bxb1 protein was generated via in vitro transcription. A DNA Nanoplasmid harboring the coding sequence of a CD19-CAR and a GFP reporter was used. The PEgRNA pairs were designed to target the exon1/intron 1 junction of the TRAC gene to utilize the endogenous TRAC promoter. Accordingly, the Nanoplasmid contained a corresponding attP sequence GGTTTGTCTGGTCAACCACCGCGGTCTCAGTGGTGTACGGTACAAACC (SEQ ID NO: 1188) and a sequence encoding a CD19 CAR connected to a sequence encoding GFP reporter by a T2A sequence. To test multiplexed Prime Editing of T cells, a single PERNA according to SEQ ID NO: 1090 (spacer SEQ ID NO: 1060, PBS SEQ ID NO: 1071, RTT SEQ ID NO: 1080, gRNA core SEQ ID NO: 590) and a nicking guide RNA (ngRNA) according to SEQ ID NO: 1130 (spacer SEQ ID NO: 1126, gRNA core SEQ ID NO: 590) were also designed to introduce a c.51delC edit in the B2M gene to knock out B2M expression.

All PERNAs and ngRNA were chemically synthesized and contained 3′ UUUU end adaptation, and further contained chemical modifications 3′ mN*mN*mN*N and 5′mN*mN*mN*N modifications, where m indicates that the nucleotide contains a 2′-O-Me modification and a * indicates a phosphorothioate bond.

Human T cells were cultured and electroporated with the Prime Editor mRNA, and TRAC 5′ PEgRNA, TRAC 3′ PEgRNA, B2M PEgRNA, and B2M ngRNA as described in Example 9. For recombinase mediated donor integration, recombinase mRNA and plasmid DNA cargo were also electroporated using Maxcyte as described in Example 9.

Following electroporation, T cells were transferred and genomic DNA was extracted as described in Example 9.

### Assessment of Prime Editing by Amplicon Sequencing

Prime Editing efficiency and indels frequency were assessed by amplicon sequencing of using the primers listed in Table 38 and 41 for TRAC and B2M amplification, respectively. These primers bear Illumina forward and reverse adapters, allowing amplicons generating in a first round of PCR (PCR1) to serve as templates for a second round of PCR (PCR2) with unique Illumina barcoding primer pairs. PCR2 products were pooled by common amplicons, purified by gel electrophoresis, and quantified by Qubit (ThermoFisher). Libraries were sequenced on an Illumina MiSeq instrument using Illumna MiSeq control software. Reads were demultiplexed by the Illumina MiSeq control software and analyzed using CRISPResso 2.

TABLE 41
Primers for detecting B2M editing

| Primer | |
|---|---|
| name | Sequence |
| B2M | ACACTCTTTCCCTACACGACGCTCTTCCGATCTNNNNTGG |
| Forward | GCACGCGTTTAATATAAG (SEQ ID NO: 1235) |
| B2M | TGGAGTTCAGACGTGTGCTCTTCCGATCTAGATCCAGCCC |
| Reverse | TGGACTAGC (SEQ ID NO: 1236) |

### Assessment of Protein Loss and Cargo Expression by Flow Cytometry

CD3 protein loss, B2M protein loss, and transgene expression was evaluated via flow cytometry detection of B2M, CD3, GFP, and CD19-CAR. The following antibodies andproteins were used: PE-conjugated anti-B2M (clone 2M2; BioLegend), BV605-conjugated anti-CD3 (clone SK7; BD Biosciences), Atto 647N-conjugated recombinant human CD19 (BioTechne). GFP signals were assessed based on flow cytometry fluorescence without antibody staining. SYTOX Blue dead cell stain (ThermoFisher). CD3 protein loss and B2M protein loss in edited cells were normalized to mock control cells and assessed as described in Example 9. Cell death was also assessed as described in Example 9. No appreciable cell death or appreciable difference in cell death between edited and mock control cells were detected in the flow cytometry.

Assessment of Cargo Integration by ddPCR

Targeted integration of DNA cargo at the TRAC locus after contacting with the Bxb1 mRNA and Nanoplasmid donor was quantified using ddPCR. Three sets of primers and probes (Table 42) were designed to detect unedited TRAC gene (ddPCR WT %), attB integration only (ddPCR attB %), and attL sequence as the result of Bxb1 mediated cargo integration (ddPCR attL %). Each sample was run as a duplexed assay consisting of an internal reference primer+probe set and an experimental primer+probe set. Reactions were set up in duplicate using the ddPCR Supermix for Probes (Bio-Rad) with 50 ng genomic DNA per assay. Droplets were generated and analyzed using the QX200 Droplet Digital PCR system (Bio-Rad).

TABLE 42
ddPCR Primers

| Primer/probe name | SEQ ID NO | Sequence |
|---|---|---|
| TRAC_ddPCR_universal_Forward | 1237 | CTTGCCAGCCCCACAGAG |
| TRAC_ddPCR_WT_Reverse | 1238 | CTGTGGGACAAGAGGATCAG |
| TRAC_ddPCR_attB_Reverse | 1239 | ATGATCCTGACGACGGAGA |
| TRAC_ddPCR_attL_Reverse | 1240 | TGTACCGTACACCACTGAGA |
| TRAC_ddPCR_universal_Probe | 1241 | TTGTCCATCACTGGCATCTGGACTCCA |
| (FAM) | | |
| TRAC_ddPCR_control_Forward | 1242 | CTCTTTATGGCCCTTGTCACT |
| TRAC_ddPCR_control_Reverse | 1243 | CCATCACACATAGACCCTGC |
| TRAC_ddPCR_control_Probe | 1244 | CATTGGACTCTAGAATGAAGCCAGGCA |
| (Cy5) | | |
| Internal reference_Forward | 1245 | CCCGATATTCCTCAGGTACT |
| Internal reference_Reverse | 1246 | GGATGAAACCCAGACACATAG |
| Internal reference_Probe | 1247 | CGTCATCCAGCAGAGAATGGAAAG |
| (TAM) | | |

Two separate experiments were performed, each with 2 biological replicates using CD3+ T cells from two healthy human donors. Sequence numbers of the TRAC-editing PEgRNAs, the efficiency of multiplexed editing of TRAC and B2M, CD3 protein loss, and CD19 CAR expression and GFP reporter expression are summarized in Tables 43 and 44. For B2M editing, both experiments used a B2M PERNA according to SEQ ID NO: 1090 and a ngRNA according to SEQ ID NO: 1130. n/a: not measured. “-”: not applicable.

TABLE 43
Experiment 1: Multiplexed prime editing and cargo expression in human T cells.

| | 5′ | 3′ | | | | | | | | | | | |
|---|---|---|---|---|---|---|---|---|---|---|---|---|---|
| | TRAC | TRAC | | | | | | | | | | | |
| | PERNA | PERNA | B2M | B2M | B2M | TRAC | TRAC | CD3 | | | ddPCR | ddPCR | ddPCR |
| Editing | SEQ | SEQ | delC | indels | protein | attB | indels | protein | GFP | CAR+ | WT | attB | attL |
| components | ID NO | ID NO | % | % | loss % | % | % | loss % | % | % | % | % | % |
| TRAC 5′ pegRNA, | 136 | 224 | 80.36 | 2.97 | 83.50 | n/a | n/a | 70.34 | 58.80 | 58.10 | 8.05 | 16.11 | 55.87 |
| TRAC 3′ pegRNA, | | | | | | | | | | | | | |
| B2M pegRNA, | | | | | | | | | | | | | |
| B2M ngRNA, | | | | | | | | | | | | | |
| CD19CAR-GFP | | | | | | | | | | | | | |
| cargo | | | | | | | | | | | | | |
| TRAC 5′ pegRNA, | 51 | 558 | 82.81 | 3.02 | 85.32 | n/a | n/a | 71.76 | 56.50 | 52.10 | 15.10 | 15.96 | 52.28 |
| TRAC 3′ pegRNA, | | | | | | | | | | | | | |
| B2M pegRNA, | | | | | | | | | | | | | |
| B2M ngRNA, | | | | | | | | | | | | | |
| CD19CAR-GFP | | | | | | | | | | | | | |
| cargo | | | | | | | | | | | | | |
| Mock | n/a | n/a | 0.01 | 0.00 | — | 0.00 | 0.00 | — | 0.96 | 0.10 | 98.98 | 1.11 | 0.95 |
| (electroporation | | | | | | | | | | | | | |
| only) | | | | | | | | | | | | | |
| B2M pegRNA, | n/a | n/a | 85.13 | 3.03 | 88.50 | 0.00 | 0.00 | 0.51 | 0.26 | 0.80 | 97.18 | 1.26 | 1.06 |
| B2M ngRNA, | 136 | 224 | 0.01 | 0.00 | 0.00 | 69.51 | 13.96 | 71.25 | 0.67 | 0.15 | 12.64 | 86.34 | 1.20 |
| TRAC 5′ pegRNA, | | | | | | | | | | | | | |
| TRAC 3′ pegRNA | | | | | | | | | | | | | |
| TRAC 5′ pegRNA, | 136 | 224 | 79.70 | 2.81 | 83.50 | 63.79 | 9.20 | 72.79 | 0.80 | 0.22 | 12.87 | 83.17 | 0.99 |
| TRAC 3′ pegRNA | | | | | | | | | | | | | |
| B2M pegRNA, | | | | | | | | | | | | | |
| B2M ngRNA | | | | | | | | | | | | | |
| CD19CAR-GFP | 136 | 224 | 0.20 | 0.00 | 0.41 | n/a | n/a | 72.88 | 56.50 | 52.10 | 16.83 | 30.69 | 52.48 |
| cargo only | | | | | | | | | | | | | |

TABLE 44
Experiment 2: Multiplexed prime editing in human T cells.

| | 5′ TRAC | 3′ TRAC | | | | | | | | | | | |
|---|---|---|---|---|---|---|---|---|---|---|---|---|---|
| | PEgRNA | PEgRNA | B2M | B2M | B2M | TRAC | TRAC | CD3 | | | | | |
| | SEQ ID | SEQ ID | delC | indels | protein | attB | indels | protein | GFP | CAR+ | ddPCR | ddPCR | ddPCR |
| Editing components | NO | NO | % | % | loss % | % | % | loss % | % | % | WT % | attB % | attL % |
| TRAC 5′ pegRNA, TRAC 3′ | 136 | 224 | 85.98 | 3.31 | 91.40 | 71.62 | 16.91 | 88.80 | n/a | n/a | N/A | N/A | N/A |
| pegRNA, B2M pegRNA, | | | | | | | | | | | | | |
| B2M ngRNA | | | | | | | | | | | | | |

### EMBODIMENTS

1. Embodiment 1. A prime editing system comprising (A) a first prime editing guide RNA (PEgRNA) or one or more polynucleotides encoding the first PEgRNA and (B) a second PEgRNA or one or more polynucleotides encoding the second PEgRNA,
   wherein the first PEgRNA comprises:
      (i) a first spacer that is complementary to a first search target sequence on a first strand of a TRAC gene,
      (ii) a first gRNA core capable of binding to a Cas9 protein; and
      (iii) a first extension arm comprising a first editing template and a first primer binding site (PBS),
   wherein the first spacer comprises at its 3′ end nucleotides 4-20 of a sequence selected from the group consisting of SEQ ID NOs: 4, 61, 88, and 150 and wherein the first PBS comprises at its 5′ end a sequence that is the reverse complement of nucleotides 13-17 of the selected sequence;
   wherein the second PERNA comprises:
      (i) a second spacer that is complementary to a second search target sequence on a second strand of the TRAC gene complementary to the first strand,
      (ii) a second gRNA core capable of binding to a Cas9 protein; and
      (iii) a second extension arm comprising a second editing template and a second PBS,
   wherein the second spacer comprises at its 3′ end nucleotides 4-20 of a sequence selected from the group consisting of SEQ ID NOs: 177, 233, 260, 287, 314, 341, 368, 414, 441, 468, 495, 522, and 566, and wherein the second PBS comprises at its 5′ end a sequence that is the reverse complement of nucleotides 13-17 of the selected sequence;
   and wherein
   (a) the first editing template comprises a region of complementarity to the second editing template;
   (b) the first editing template comprises nucleotides 8-17 of the selected sequence for the second spacer, and the second editing template comprises nucleotides 8-17 of the selected sequence for the first spacer; or
   (c) the first editing template comprises nucleotides 8-17 of the selected sequence for the second spacer and a region of complementarity to the second editing template, and the second editing template comprises nucleotides 8-17 of the selected sequence for the first spacer and a region of complementarity to the first editing template.
2. Embodiment 2. The prime editing system of embodiment 1, wherein the selected sequence for the first spacer is SEQ ID NO: 4 or 88.
3. Embodiment 3. The prime editing system of embodiment 2, wherein the selected sequence for the first spacer is SEQ ID NO: 88.
4. Embodiment 4. The prime editing system of any one of embodiments 1-2, wherein the selected sequence for the second spacer is SEQ ID NO: 177, 368, or 522.
5. Embodiment 5. The prime editing system of embodiment 4, wherein the selected sequence for the second spacer is SEQ ID NO: 177.
6. Embodiment 6. The prime editing system of any one of embodiments 1-5, wherein the first spacer and/or the second spacer is from 16 to 22 nucleotides in length.
7. Embodiment 7. The prime editing system of any one of embodiments 1-6, wherein the first spacer and/or the second spacer is 20 nucleotides in length and comprises the selected sequence.
8. Embodiment 8. The prime editing system of any one of embodiments 1-7, wherein the first PBS is 8-17 nucleotides in length and comprises at its 5′ end a sequence that is the reverse complement of nucleotides 10-17, 9-17, 8-17, 7-17, 6-17, 5-17, 4-17, 3-17, 2-17, or 1-17 of the selected sequence for the first spacer.
9. Embodiment 9. The prime editing system of embodiment 8, wherein the first PBS is 8-13 nucleotides in length.
10. Embodiment 10. The prime editing system of embodiment 9, wherein the first PBS is 10, 11, or 12 nucleotides in length.
11. Embodiment 11. The prime editing system of any one of embodiments 1-10, wherein the second PBS is 7-17 nucleotides in length and comprises at its 5′ end a sequence that is the reverse complement of nucleotides 11-17, 10-17, 9-17, 8-17, 7-17, 6-17, 5-17, 4-17, 3-17, 2-17, or 1-17 of the selected sequence for the second spacer.
12. Embodiment 12. The prime editing system of embodiment 11, wherein the second PBS is 8-13 nucleotides in length.
13. Embodiment 13. The prime editing system of embodiment 11, wherein the second PBS is 11, 12, or 13 nucleotides in length.
14. Embodiment 14. The prime editing system of any one of embodiment 1-13, wherein the first gRNA core and the second gRNA core comprise the same sequence.
15. Embodiment 15. The prime editing system of embodiment 14, wherein the first gRNA core, the second gRNA core, or both comprise SEQ ID NO: 590.
16. Embodiment 16. The prime editing system of any one of embodiments 1-15, wherein the first spacer, the first gRNA core, the first editing template, and the first PBS form a contiguous sequence in a single molecule.
17. Embodiment 17. The prime editing system of embodiment 16, wherein the first PEgRNA comprises from 5′ to 3′ the first spacer, the first gRNA core, the first editing template, and the first PBS.
18. Embodiment 18. The prime editing system of any one of embodiments 1-17, wherein the second spacer, the second gRNA core, the second editing template, and the second PBS form a contiguous sequence in a single molecule.
19. Embodiment 19. The prime editing system of embodiment 18, wherein the second pegRNA comprises from 5′ to 3′ the second spacer, the second gRNA core, the second editing template, and the second PBS.
20. Embodiment 20. The prime editing system of any one of embodiments 1-19, wherein the first editing template comprises a region of complementarity to the second editing template.
21. Embodiment 21. The prime editing system of embodiment 20, wherein the first editing template and the second editing template each encodes all or a fragment of a recombinase recognition sequence (RSS) or the reverse complement thereof, wherein the first editing template encodes at least a 5′ portion of the RSS or the reverse complement thereof, wherein the second editing template encodes at least a 3′ portion of the RSS or the reverse complement thereof, and wherein at least 10 nucleotides at the 5′ end of the first and the second editing templates have perfect reverse complementarity to each other.
22. Embodiment 22. The prime editing system of embodiment 21, wherein at least 15, 20, 25, or 30 nucleotides at the 5′ ends of the first and the second editing templates have perfect reverse complementarity to each other, optionally wherein at least 20, 21, 22, 23, 24, 25, 26, or 27 nucleotides at the 5′ ends of the first and the second editing templates have prefect reverse complementarity to each other.
23. Embodiment 23. The prime editing system of embodiment 21 or 22, wherein the first editing template encodes the RSS.
24. Embodiment 24. The prime editing system of any one of embodiments 21-23, wherein the second editing template encodes the RSS.
25. Embodiment 25. The prime editing system of any one of embodiments 21-24, wherein the RSS is an attB sequence recognized by a Bxb1 recombinase.
26. Embodiment 26. The prime editing system of any one of embodiments 21-24, wherein the RSS is an attP sequence recognized by a Bxb1 recombinase.
27. Embodiment 27. The prime editing system of any one of embodiments 21-24, wherein the RSS is an attB sequence recognized by a Pa01 recombinase.
28. Embodiment 28. prime editing system of any one of embodiments 21-24, wherein the RSS is an attP sequence recognized by a Pa01 recombinase.
29. Embodiment 29. The prime editing system of any one of embodiments 20-25, wherein the first editing template comprises an RTT #1 from Table 6 and the second editing template comprises an RTT #2 from the same RTT Pair in Table 6, or wherein the first editing template comprises an RTT #2 from Table 6 and the second editing template comprises an RTT #1 from the same RTT Pair in Table 6.
30. Embodiment 30. The prime editing system of any one of embodiments 20-25, wherein the first editing template comprises SEQ ID NO: 24 and the second editing template comprises SEQ ID NO: 196.
31. Embodiment 31. The prime editing system of any one of embodiments 20-25, wherein the first editing template comprises SEQ ID NO: 23 and the second editing template comprises SEQ ID NO: 196.
32. Embodiment 32. The prime editing system of any one of embodiments 20-25, wherein the first editing template comprises SEQ ID NO: 24 and the second editing template comprises SEQ ID NO: 106.
33. Embodiment 33. The prime editing system of any one of embodiments 20-25, wherein the first editing template comprises SEQ ID NO: 27 and the second editing template comprises SEQ ID NO: 107.
34. Embodiment 34. The prime editing system of any one of embodiments 20-25, wherein the first editing template comprises SEQ ID NO: 106 and the second editing template comprises SEQ ID NO: 24.
35. Embodiment 35. The prime editing system of any one of embodiments 20-25, wherein the first editing template comprises SEQ ID NO: 107 and the second editing template comprises SEQ ID NO: 27.
36. Embodiment 36. The prime editing system of any one of embodiments 20-25, wherein the first editing template comprises SEQ ID NO: 25 and the second editing template comprises SEQ ID NO: 197.
37. Embodiment 37. The prime editing system of any one of embodiments 20-25, wherein the first editing template comprises SEQ ID NO: 28 and the second editing template comprises SEQ ID NO: 199.
38. Embodiment 38. The prime editing system of any one of embodiments 20-25, wherein the first editing template comprises SEQ ID NO: 22 and the second editing template comprises SEQ ID NO: 195.
39. Embodiment 39. The prime editing system of any one of embodiments 20-25, wherein the first editing template comprises SEQ ID NO: 26 and the second editing template comprises SEQ ID NO: 198.
40. Embodiment 40. The prime editing system of any one of embodiments 20-25, wherein the first editing template comprises a 5′ fragment of an RTT listed in Table 6 and wherein the second editing template comprises a full length or 5′ fragment of the corresponding RTT pair and wherein at least 10 nucleotides at the 5′ ends of the first and the second editing templates have perfect reverse complementarity to each other.
41. Embodiment 41. The prime editing system of any one of embodiments 20-25, wherein the second editing template comprises a 5′ fragment of an RTT listed in Table 6 and wherein the first editing template comprises a full length or 5′ fragment of the corresponding RTT pair and wherein at least 10 nucleotides at the 5′ ends of the first and the second editing templates have perfect reverse complementarity to each other.
42. Embodiment 42. The prime editing system of embodiment 40 or 41, wherein at least 15, 20, 25, or 30 nucleotides at the 5′ ends of the first and the second editing templates have perfect reverse complementarity to each other. optionally wherein at least 20, 21, 22, 23, 24, 25, 26, or 27 nucleotides at the 5′ ends of the first and the second editing templates have prefect reverse complementarity to each other.
43. Embodiment 43. The prime editing system of embodiment 40 or 41, wherein the length of the region of complementarity of the first editing template is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% of the length of the first editing template, optionally wherein the length of the region of complementarity of the first editing template is at least 52%, at least 53%, or at least 55% of the length of the first editing template.
44. Embodiment 44. The prime editing system of embodiment 40 or 41, wherein the length of the region of complementarity of the second editing template is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% of the length of the second editing template, optionally wherein the length of the region of complementarity of the second editing template is at least 52%, at least 53%, or at least 55% of the length of the second editing template.
45. Embodiment 45. The prime editing system of any one of embodiments 20-44, wherein:
   (a) the first spacer comprises SEQ ID NO: 4, and the first PBS comprises SEQ ID NO: 13, or the first spacer comprises SEQ ID NO: 88, and the first PBS comprises SEQ ID NO: 96, or the first spacer comprises SEQ ID NO: 88, and the first PBS comprises SEQ ID NO: 157, or the first spacer comprises SEQ ID NO: 88, and the first PBS comprises SEQ ID NO: 99, or the first spacer comprises SEQ ID NO: 88, and the first PBS comprises SEQ ID NO: 97; and
   (b) the second spacer comprises SEQ ID NO: 177, and the second PBS comprises SEQ ID NO: 188, or
   the second spacer comprises SEQ ID NO: 368, and the second PBS comprises SEQ ID NO: 376.
46. Embodiment 46. The prime editing system of any one of embodiments 20-44, wherein:
   (a) the first spacer comprises SEQ ID NO: 4, and the first PBS has the sequence according to SEQ ID NO: 14 or SEQ ID NO: 15; or
   the first spacer comprises SEQ ID NO: 88, and the first PBS has the sequence according to SEQ ID NO: 96 or SEQ ID NO: 98;
   and
   (b) the second spacer comprises SEQ ID NO: 177, and the second PBS has the sequence according to SEQ ID NO: 186 or SEQ ID NO: 188,
   the second spacer comprises SEQ ID NO: 368, and the second PBS has the sequence according to SEQ ID NO: 373 or SEQ ID NO: 374; or
   the second spacer comprises SEQ ID NO: 522, and the second PBS has the sequence according to SEQ ID NO: 531 or 533.
47. Embodiment 47. The prime editing system of embodiment 46, wherein the first spacer comprises SEQ ID NO: 88, and the first PBS has the sequence according to SEQ ID NO: 96, and wherein the second spacer comprises SEQ ID NO: 177, and the second PBS has the sequence according to SEQ ID NO: 186.
48. Embodiment 48. The prime editing system of embodiment 46 or 47, wherein the first editing template comprises SEQ ID NO: 27 and the second editing template comprises SEQ ID NO: 107.
49. Embodiment 49. The prime editing system of embodiment 46 or 47, wherein the first editing template comprises SEQ ID NO: 107 and the second editing template comprises SEQ ID NO: 27.
50. Embodiment 50. The prime editing system of embodiment 20, wherein the first PEgRNA comprises SEQ ID NO: 118 and the second PEgRNA comprises SEQ ID NO: 1135.
51. Embodiment 51. The prime editing system of embodiment 20, wherein the first PEgRNA comprises SEQ ID NO: 118 and the second PEgRNA comprises SEQ ID NO: 546.
52. Embodiment 52. The prime editing system of embodiment 20, wherein the first PEgRNA comprises SEQ ID NO: 118 and the second PEgRNA comprises SEQ ID NO: 1136.
53. Embodiment 53. The prime editing system of embodiment 20, wherein the first PEgRNA comprises SEQ ID NO: 118 and the second PEgRNA comprises SEQ ID NO: 1137.
54. Embodiment 54. The prime editing system of embodiment 20, wherein the first PEgRNA comprises SEQ ID NO: 118 and the second PEgRNA comprises SEQ ID NO: 1138.
55. Embodiment 55. The prime editing system of embodiment 20, wherein the first PEgRNA comprises SEQ ID NO: 118 and the second PEgRNA comprises SEQ ID NO: 1139.
56. Embodiment 56. The prime editing system of embodiment 20, wherein the first PEgRNA comprises SEQ ID NO: 118 and the second PEgRNA comprises SEQ ID NO: 1140.
57. Embodiment 57. The prime editing system of embodiment 20, wherein the first PEgRNA comprises SEQ ID NO: 118 and the second PEgRNA comprises SEQ ID NO: 1141.
58. Embodiment 58. The prime editing system of embodiment 20, wherein the first PEgRNA comprises SEQ ID NO: 118 and the second PEgRNA comprises SEQ ID NO: 1142.
59. Embodiment 59. The prime editing system of embodiment 20, wherein the first PEgRNA comprises SEQ ID NO: 123 and the second PEgRNA comprises SEQ ID NO: 1143.
60. Embodiment 60. The prime editing system of embodiment 20, wherein the first PEgRNA comprises SEQ ID NO: 123 and the second PEgRNA comprises SEQ ID NO: 1144.
61. Embodiment 61. The prime editing system of embodiment 20, wherein the first PEgRNA comprises SEQ ID NO: 123 and the second PEgRNA comprises SEQ ID NO: 1145.
62. Embodiment 62. The prime editing system of embodiment 20, wherein the first PERNA comprises SEQ ID NO: 36 and the second PEgRNA comprises SEQ ID NO: 1146.
63. Embodiment 63. The prime editing system of embodiment 20, wherein the first PEgRNA comprises SEQ ID NO: 36 and the second PEgRNA comprises SEQ ID NO: 1147.
64. Embodiment 64. The prime editing system of embodiment 20, wherein the first PEgRNA comprises SEQ ID NO: 36 and the second PEgRNA comprises SEQ ID NO: 1148.
65. Embodiment 65. The prime editing system of embodiment 20, wherein the first PEgRNA comprises SEQ ID NO: 36 and the second PEgRNA comprises SEQ ID NO: 1149.
66. Embodiment 66. The prime editing system of embodiment 20, wherein the first PEgRNA comprises SEQ ID NO: 1132 and the second PEgRNA comprises SEQ ID NO: 1150.
67. Embodiment 67. The prime editing system of embodiment 20, wherein the first PEgRNA comprises SEQ ID NO: 1133 and the second PEgRNA comprises SEQ ID NO: 1151.
68. Embodiment 68. The prime editing system of embodiment 20, wherein the first PEgRNA comprises SEQ ID NO: 1134 and the second PEgRNA comprises SEQ ID NO: 1152.
69. Embodiment 69. The prime editing system of embodiment 20, wherein the first PEgRNA comprises SEQ ID NO: 136 and the second PEgRNA comprises SEQ ID NO: 224.
70. Embodiment 70. The prime editing system of embodiment 20, wherein the first PEgRNA comprises SEQ ID NO: 51 and the second PEgRNA comprises SEQ ID NO: 556.
71. Embodiment 71. The prime editing system of embodiment 20, wherein the first PEgRNA comprises SEQ ID NO: 126 and the second PERNA comprises SEQ ID NO: 220.
72. Embodiment 72. The prime editing system of embodiment 20, wherein the first PEgRNA comprises SEQ ID NO: 44 and the second PEgRNA comprises SEQ ID NO: 550.
73. Embodiment 73. The prime editing system of embodiment 20, wherein the first PEgRNA comprises SEQ ID NO: 111 and the second PEgRNA comprises SEQ ID NO: 1251.
74. Embodiment 74. The prime editing system of embodiment 20, wherein the first PEgRNA comprises a sequence selected from the group consisting of SEQ ID NOs: 30, 31, 32, 33, 34, 36, 38, 39, 43, 44, 49, 50, 51, 52, 53, 79, 80, 82, 109, 111, 112, 114, 115, 118, 120, 122, 123, 125, 126, 129, 130, 134, 135, 136, 140, 141, 143, 168, 169, 171, 592, 593, 594, and 1132; and wherein the second PEgRNA comprises a sequence selected from the group consisting of SEQ ID NOs: 203, 207, 210, 211, 212, 215, 217, 219, 220, 221, 222, 224, 226, 228, 251, 252, 254, 278, 279, 281, 305, 306, 308, 332, 333, 335, 359, 360, 362, 388, 390, 392, 395, 398, 397, 400, 401, 403, 404, 405, 408, 410, 432, 433, 435, 459, 460, 462, 486, 487, 489, 513, 514, 516, 541, 542, 543, 545, 546, 547, 549, 550, 552, 554, 555, 556, 558, 561, 562, 584, 585, 587, 591, 595, 597, 599, 601, 1127, 1128, 1135, 1136, 1137, 1138, 1139, 1140, and 1141.
75. Embodiment 75. The prime editing system of embodiment 20, wherein the first PEgRNA comprises a sequence selected from the group consisting of SEQ ID NOs: 30, 44, 109, and 126; and wherein the second PEgRNA comprises a sequence selected from the group consisting of SEQ ID NOs: 207, 221, 388, and 400.
76. Embodiment 76. The prime editing system of embodiment 20, wherein the first PERNA comprises a sequence selected from the group consisting of SEQ ID NOs: 118, 136, 141, 51, and 53; and wherein the second PEgRNA comprises a sequence selected from the group consisting of SEQ ID NOs: 1140, 226, 403, 558, 556, and 224.
77. Embodiment 77. The prime editing system of embodiment 76, wherein the first PEgRNA comprises SEQ ID NO: 136, and wherein the second PEgRNA comprises SEQ ID NO: 224.
78. Embodiment 78. The prime editing system of any one of embodiments 1-77, wherein the first PEgRNA and/or the second PEgRNA further comprises a 3′ motif, optionally wherein the 3′ motif is connected to the 3′ end of the first PBS or the second PBS via a linker.
79. Embodiment 79. The prime editing system of any one of embodiments 1-78, wherein the first PEgRNA and/or the second PERNA further comprises 5′mN*mN*mN* and 3′ mN*mN*mN*N modifications, where m indicates that the nucleotide contains a 2′-O-Me modification and a * indicates the presence of a phosphorothioate bond.
80. Embodiment 80. The prime editing system of any one of embodiments 1-79, further comprising a prime editor or one or more polynucleotides encoding the prime editor, wherein the prime editor comprises (a) a Cas9 nickase having a nuclease inactivating mutation in a HNH domain and (b) a reverse transcriptase.
81. Embodiment 81. The prime editing system of embodiment 80, wherein the Cas9 nickase comprises an amino acid sequence having at least 80%, 85%, 90%, 95%, 96%, 97%, 98%, 99%, or 100% identity to SEQ ID NO: 1007.
82. Embodiment 82. The prime editing system of embodiment 80 or 81, wherein the reverse transcriptase comprises an amino acid sequence having at least 80%, 85%, 90%, 95%, 96%, 97%, 98%, 99%, or 100% identity to SEQ ID NO: 1003.
83. Embodiment 83. The prime editing system of any one of embodiments 80-82, wherein the prime editor is a fusion protein.
84. Embodiment 84. The prime editing system of embodiment 83, wherein the fusion protein comprises SEQ ID NO: 1033.
85. Embodiment 85. The prime editing system of any one of embodiments 79-84, wherein the one or more polynucleotides encoding the prime editor comprise (a) a first sequence encoding an N-terminal portion of the Cas9 nickase and an intein-N and (b) a second sequence encoding an intein-C, a C-terminal portion of the Cas9 nickase, and the reverse transcriptase.
86. Embodiment 86. The prime editing system of any one of embodiments 21-85, further comprising a recombinase that recognizes the one or more RSSs or one or more polynucleotides encoding the recombinase.
87. Embodiment 87. The prime editing system of embodiment 86, wherein the recombinase is Bxb1 or Pa01.
88. Embodiment 88. The prime editing system of embodiment 86, wherein the recombinase is a Bxb1 comprising an amino acid sequence having at least 80%, 85%, 90%, 95%, 96%, 97%, 98%, 99%, or 100% identity to SEQ ID NO: 1131.
89. Embodiment 89. The prime editing system of any one of embodiment 81, 82, or 88, wherein the sequence identities are determined by Needleman-Wunsch alignment of two protein sequences with Gap Costs set to Existence: 11 Extension: 1 where percent identity is calculated by dividing the number of identities by the length of the alignment.
90. Embodiment 90. The prime editing system of embodiment 86 or 88, wherein the recombinase is fused or linked to the prime editor.
91. Embodiment 91. The prime editing system of any one of embodiments 86-90, further comprising a DNA polynucleotide that comprises (a) a donor sequence and (b) a second RSS recognized by the recombinase.
92. Embodiment 92. The prime editing system of embodiment 91, wherein (i) the RSS comprises a Bxb1 attB sequence provided in Table 5, and the second RSS comprises a corresponding attP sequence provided in Table 5, or (ii) the RSS comprises a Bxb1 attP sequence provided in Table 5, and the second RSS comprises a corresponding attB sequence provided in Table 5.
93. Embodiment 93. The prime editing system of embodiment 91, wherein the RSS sequence comprises SEQ ID NO: 1187 and the second RSS comprises SEQ ID NO: 1188.
94. Embodiment 94. The prime editing system of embodiment 91, wherein the donor sequence comprises an open reading frame that encodes a polypeptide.
95. Embodiment 95. The prime editing system of embodiment 94, wherein the donor sequence encodes a chimeric antigen receptor (CAR).
96. Embodiment 96. The prime editing system of embodiment 94, wherein the donor sequence encodes a CD19 CAR.
97. Embodiment 97. The prime editing system of embodiment 94, wherein the donor sequence comprises a splice acceptor sequence.
98. Embodiment 98. The prime editing system of any one of embodiments 80-85, comprising one or more vectors that comprises the one or more polynucleotides encoding the first PEgRNA, the one or more polynucleotides encoding the second PEgRNA, and the one or more polynucleotides encoding the prime editor.
99. Embodiment 99. The prime editing system of any one of embodiment 80-85, comprising one or more vectors that comprises the one or more polynucleotides encoding the first PERNA, the one or more polynucleotides encoding the second PEgRNA, the one or more polynucleotides encoding the prime editor, the one or more polynucleotides encoding the recombinase, and the donor sequence.
100. Embodiment 100. The prime editing system of embodiment 99, wherein the one or more vectors are AAV vectors.
101. Embodiment 101. The prime editing system of any one of embodiments 80-99, wherein the one or more polynucleotides encoding the prime editor and/or the one or more polynucleotides encoding the recombinase are mRNA.
102. Embodiment 102. The prime editing system of any one of embodiments 1-101, wherein the system further comprises a third PEgRNA or a nucleic acid encoding the third PEgRNA, wherein the third PEgRNA comprises:
   (i) a third spacer that is complementary to a search target sequence on a first strand of a β2-microglobulin (B2M) gene;
   (ii) a third gRNA core capable of binding to a Cas9 protein; and
   (iii) a third extension arm comprising:
      (a) a third editing template that comprises a region of complementarity to an editing target sequence on a second strand of the B2M gene, and
      (b) a third primer binding site (PBS) that comprises a sequence that is a reverse complement of a portion of the third spacer,
      wherein the first and second strand of the B2M gene are complementary to each other, and wherein the third editing template encodes one or more nucleotide changes compared to the editing target sequence.
103. Embodiment 103. The prime editing system of embodiment 102, wherein the third spacer comprises at its 3′ end SEQ ID NO: 1063.
104. Embodiment 104. The prime editing system of embodiment 103, wherein the third PBS comprises at its 5′ end a sequence that is a reverse complement of nucleotides 10-14 of SEQ ID NO: 1063.
105. Embodiment 105. The prime editing system of any one of embodiment 102-104, wherein the editing template encodes one or more in-frame stop codons or the complement thereof in the B2M gene. 106. Embodiment 106. The prime editing system of embodiment 105, wherein the one or more in-frame stop codons comprise a nonsense mutation in the B2M gene.
107. Embodiment 107. The prime editing system of embodiment 105, wherein the one or more in-frame stop codons comprise an insertion in the B2M gene.
108. Embodiment 108. The prime editing system of embodiment 105, wherein the editing template encodes an insertion of two in-frame stop codons in the B2M gene.
109. Embodiment 109. The prime editing system of embodiment 108, wherein the insertion is TAATAA or TTATTA.
110. Embodiment 110. The prime editing system of any one of embodiments 102-104, wherein the editing template encodes a frameshift mutation in the B2M gene.
111. Embodiment 111. The prime editing system of embodiment 110, wherein the frameshift mutation is an insertion.
12. Embodiment 112. The prime editing system of embodiment 111, wherein the insertion is c.50insG or the complement thereof.
113. Embodiment 113. The prime editing system of embodiment 110, wherein the frameshift mutation is a deletion.
114. Embodiment 114. The prime editing system of embodiment 113, wherein the deletion is c.51delC or the complement thereof.
115. Embodiment 115. The prime editing system of any one of embodiments 1-101, wherein the system further comprises a third PEgRNA or a nucleic acid encoding the third PEgRNA, wherein the third PEgRNA comprises:
a. a third spacer comprising at its 3′ end SEQ ID NO: 1063;
b. a third gRNA core capable of binding to a Cas9 protein; and
c. a third extension arm comprising:
   i. a third editing template comprising at its 3′ end: (A) nucleotides 13-24 of SEQ ID NO: 1079, (B) nucleotides 12-20 of SEQ ID NO: 1085, or (C) nucleotides 7-17 of SEQ ID NO: 1089, and
   ii. a third primer binding site (PBS) comprising at its 5′ end a sequence that is a reverse complement of nucleotides 10-14 of SEQ ID NO: 1063.
116. Embodiment 116. The prime editing system of any one of embodiments 102-115, wherein the third spacer is from 17-22 nucleotides in length.
117. Embodiment 117. The prime editing system of embodiment 116, wherein the third spacer comprises at its 3′ end any one of SEQ ID NOs: 1060-1063.
118. Embodiment 118. The prime editing system of embodiments 115-117, wherein the third editing template comprises at its 3′ end nucleotides 13-24 of SEQ ID NO: 1077.
119. Embodiment 119. The prime editing system of embodiment 118, wherein the third editing template comprises at its 3′ end SEQ ID NO: 1077.
120. Embodiment 120. The prime editing system of embodiment 119, wherein the third editing template comprises at its 3′ end any one of SEQ ID NOs: 1078 or 1079.
121. Embodiment 121. The prime editing system of any one of embodiments 115-117, wherein the third editing template comprises at its 3′ end nucleotides 12-20 of SEQ ID NO: 1085.
122. Embodiment 122. The prime editing system of embodiment 121, wherein the third editing template comprises at its 3′ end SEQ ID NO: 1081.
123. Embodiment 123. The prime editing system of embodiment 122, wherein the third editing template comprises at its 3′ end any one of SEQ ID Nos: 1080, 1082-1085.
124. Embodiment 124. The PEgRNA of any one of embodiments 115-117, wherein the third editing template comprises at its 3′ end nucleotides 7-17 of SEQ ID NO: 1089.
125. Embodiment 125. The prime editing system of embodiment 124, wherein the third editing template comprises at its 3′ end any one of SEQ ID NOs: 1087-1089.
126. Embodiment 126. The prime editing system of any one of embodiments 102-125, wherein the third editing template has a length of 24 nucleotides or less.
127. Embodiment 127. The prime editing system of any one of embodiments 102-125, wherein the third editing template has a length of 20 nucleotides or less.
128. Embodiment 128. The prime editing system of embodiment 127, wherein the third editing template has a length of 10 to 20 nucleotides.
129. Embodiment 129. The prime editing system of embodiment 127, wherein the third editing template has a length of 12 to 20 nucleotides.
130. Embodiment 130. The prime editing system of embodiment 127, wherein the third editing template has a length of 11 to 17 nucleotides.
131. Embodiment 131. The prime editing system of embodiment 126, wherein the third editing template is 16 to 24 nucleotides in length.
132. Embodiment 132. The prime editing system of any one of embodiments 102-131, wherein the third PBS comprises at its 3′ end a sequence set forth in any one of sequence numbers 1064-1076.
133. Embodiment 133. The prime editing system of any one of embodiments 102-132, wherein the third PBS has a length of 17 nucleotides or less.
134. Embodiment 134. The prime editing system of embodiment 133, wherein the third PBS is 8 to 15 nucleotides in length.
135. Embodiment 135. The prime editing system of embodiment 133, wherein the third PBS is 8 to 14 nucleotides in length.
136. Embodiment 136. The prime editing system of embodiment 133, wherein the third PBS is 12 nucleotides in length.
137. Embodiment 137. The prime editing system of any one of embodiments 102-136, wherein the third spacer, the third gRNA core, the third editing template, and the third PBS form a contiguous sequence in a single molecule.
138. Embodiment 138. The prime editing system of embodiment 137, wherein the single molecule comprises from 5′ to 3′, the third spacer, the third gRNA core, the third editing template and the third PBS.
139. Embodiment 139. The prime editing system of any one of embodiments 102-138, wherein the third PEgRNA comprises a sequence selected from any one SEQ ID NOs: 1090-1120.
140. Embodiment 140. The prime editing system of any one of embodiments 102-139, wherein the system further comprises a nick guide RNA (ngRNA), or a nucleic acid encoding the ngRNA, wherein the ngRNA comprises: (i) a ngRNA spacer that is complementary to a ngRNA target sequence on the second strand of the B2M gene; and (ii) an ngRNA core capable of binding a Cas9 protein.
141. Embodiment 141. The prime editing system of embodiment 140, wherein the ngRNA spacer comprises at its 3′ end a sequence corresponding to nucleotides 4-20, 3-20, 2-20, or 1-20 of any one of SEQ ID NOs: 1121-1126.
142. Embodiment 142. The prime editing system of embodiment 140, wherein ngRNA spacer comprises at its 3′ end any one of SEQ ID NOs: 1121-1126.
143. Embodiment 143. The prime editing system of embodiment 140, wherein the ngRNA spacer comprises at its 3′ end SEQ ID NO: 1126.
144. Embodiment 144. The prime editing system of embodiment 140, wherein the one or more nucleotide encoded by the editing template is c.51delC or the complement thereof, and wherein the ngRNA spacer comprises at its 3′ end sequence corresponding to nucleotides 4-20, 3-20, 2-20, or 1-20 of SEQ ID NO: 1124.
145. Embodiment 145. The prime editing system of embodiment 140, wherein the one or more nucleotide encoded by the editing template is c.50insG or the complement thereof, and wherein the ngRNA spacer comprises at its 3′ end sequence corresponding to nucleotides 4-20, 3-20, 2-20, or 1-20 of SEQ ID NO: 1125 or 1126.
146. Embodiment 146. The prime editing system of embodiment 140, wherein the ngRNA comprises SEQ ID NO: 1129.
147. Embodiment 147. An LNP comprising the prime editing system of any one of embodiments 1-146.
148. Embodiment 148. A pharmaceutical composition comprising the prime editing system of any one of embodiment 1-146 or the LNP of embodiment 147 and a pharmaceutically acceptable excipient.
149. Embodiment 149. A method of editing a TRAC gene, the method comprising contacting the TRAC gene with (a) the prime editing system of any one of embodiments 1-79 and a prime editor comprising a Cas9 nickase having a nuclease inactivation mutation in a HNH domain and a reverse transcriptase or (b) the prime editing system of any one of embodiments 80-85.
150. Embodiment 150. The method of embodiment 149, further comprising contacting the TRAC gene with a recombinase or one or more polynucleotides encoding the recombinase and a DNA polynucleotide comprising (a) a donor sequence and (b) one or more recombinase recognition sequences recognized by the recombinase.
151. Embodiment 151. A method of inserting a donor sequence into a TRAC gene, the method comprising contacting the TRAC gene with the prime editing system of any one of embodiments 1-101 or the LNP of embodiment 147.
152. Embodiment 152. A method of making a modified cell, the method comprising contacting a cell with the prime editing system of any one of embodiments 1-146 or the LNP of embodiment 147.
153. Embodiment 153. The method of embodiment 151, wherein the TRAC gene is in a cell. 154. Embodiment 154. The method of embodiment 152 or 153, wherein the cell is a mammalian cell. 155. Embodiment 155. The method of embodiment 152 or 153, wherein the cell is a human cell.
156. Embodiment 156. The method of any one of embodiments 153-155, wherein the cell is an immune cell, optionally wherein the cell is a T cell.
157. Embodiment 157. The method of any one of embodiments 153-156, wherein the cell is in a subject.
158. Embodiment 158. The method of any one of embodiments 153-156, wherein the cell is from a subject.
159. Embodiment 159. The method of embodiment 157 or 158, wherein the subject is a human.
160. Embodiment 160. The method of any one of embodiments 149-159, further comprising editing a B2M gene.
161. Embodiment 161. The method of embodiment 160, wherein the editing comprises contacting the B2M gene with the prime editing system of any one of embodiments 102-146 and a prime editor comprising a Cas9 nickase having a nuclease inactivation mutation in a HNH domain and a reverse transcriptase.
162. Embodiment 162. A cell generated by the method of any one of embodiments 149-161.
163. Embodiment 163. A T cell comprising an edited TRAC gene that comprises GGCTTGTCGACGACGGCGGTCTCAGTGGTGTACGGTACAAACC (SEQ ID NO: 1046) and/or GGTTTGTCTGGTCAACCACCGCGGTCTCCGTCGTCAGGATCAT (SEQ ID NO: 1047) relative to a wildtype TRAC gene.
164. Embodiment 164. The T cell of embodiment 163, wherein the edited TRAC gene comprises an insert sequence comprising, from 5′ to 3′, GGCTTGTCGACGACGGCGGTCTCAGTGGTGTACGGTACAAACC (SEQ ID NO: 1046), a donor sequence, and GGTTTGTCTGGTCAACCACCGCGGTCTCCGTCGTCAGGATCAT (SEQ ID NO: 1047).
165. Embodiment 165. The T cell of embodiment 163, wherein the edited TRAC gene comprises an insert sequence comprising, from 5′ to 3′, GGTTTGTCTGGTCAACCACCGCGGTCTCCGTCGTCAGGATCAT (SEQ ID NO: 1047), a donor sequence, and GGCTTGTCGACGACGGCGGTCTCAGTGGTGTACGGTACAAACC (SEQ ID NO: 1046).
166. Embodiment 166. The T cell of embodiment 164 or 165, wherein the donor sequence encodes a chimeric antigen receptor (CAR), optionally wherein the donor encodes a CD19 CAR.
167. Embodiment 167. The T cell of embodiment 164 or 165, wherein the insert sequence is between a first chromosome location and a second chromosome location, wherein the first chromosome location is selected from the group consisting of human chromosome 14 positions 22547458, 22547457, 22547449, and 22547448, and wherein the second chromosome location is selected from the group consisting of human chromosome 14 positions 22547533, 22547523, 22547491, 22547528, 22547497, 22547579, 22547522, 22547485, 22547506, 22547560, 22547505, 22547529, and 22547490.
168. Embodiment 168. The T cell of any one of embodiments 164-167, wherein the insert sequence is between human chromosome 14 positions 22547458 and 22547533.
169. Embodiment 169. The T cell of any one of embodiments 164-167, wherein the insert sequence is between human chromosome 14 positions 22547458 and 22547522.
170. Embodiment 170. The T cell of any one of embodiments 164-167, wherein the insert sequence is between human chromosome 14 positions 22547458 and 22547529.
171. Embodiment 171. The T cell of any one of embodiments 164-167, wherein the insert sequence is between human chromosome 14 positions 22547449 and 22547533.
172. Embodiment 172. The T cell of any one of embodiments 164-167, wherein the insert sequence is between human chromosome 14 positions 22547449 and 22547522.
173. Embodiment 173. The T cell of any one of embodiments 164-167, wherein the insert sequence is between human chromosome 14 positions 22547449 and 22547529.
174. Embodiment 174. The T cell of any one of embodiments 163-173, wherein the T cell further comprises a premature stop codon relative to a wildtype B2M gene.
175. Embodiment 175. The T cell of any one of embodiments 163-173, wherein the T cell further comprises a B2M gene comprising a c.51delC edit relative to a wildtype B2M gene.
176. Embodiment 176. The T cell of any one of embodiments 163-173, wherein the T cell further comprises a B2M gene comprising a c.50insG edit relative to a wildtype B2M gene.
177. Embodiment 177. The T cell of any one of embodiments 163-173, wherein the T cell further comprises a B2M gene comprising a c.54insTAATAA edit relative to a wildtype B2M gene.
178. Embodiment 178. The T cell of any one of embodiments 167-177, wherein the human chromosome locations and coding sequence locations are as set forth in Genome Reference Consortium Human Build 38 (GrCh38).
179. Embodiment 179. A prime editing system comprising (a) a prime editing guide RNA (PEgRNA) or one or more polynucleotides encoding the PEgRNA, wherein the PEgRNA comprises: (i) a spacer that is complementary to a search target sequence on a first strand of a target gene, (ii) a gRNA core capable of binding to a Cas9 nickase, and (iii) an extension arm comprising a primer binding site (PBS) that comprises a region of complementarity to a second strand of the target gene and an editing template encoding a recombinase recognition sequence (RSS) recognized by a Pa01 recombinase; (b) a prime editor comprising the Cas9 nickase capable of nicking the second strand of the target gene at a nick site and a reverse transcriptase, or one or more polynucleotides encoding the prime editor; and (c) a Pa01 recombinase or one or more polynucleotides encoding the Pa01 recombinase.
180. Embodiment 180. The prime editing system of embodiment 179, wherein the PBS comprises a region of complementarity to a region upstream of the nick site.
181. Embodiment 181. The prime editing system of any one of embodiments 179-180, wherein the editing template comprises a region of complementarity to a region downstream of the nick site. 182. Embodiment 182. The prime editing system of any one of embodiments 179-181, wherein the Cas9 nickase comprises a nuclease inactivation mutation in a HNH domain.
183. Embodiment 183. A prime editing system comprising (A) a first prime editing guide RNA (PEgRNA) or one or more polynucleotides encoding the first PEgRNA, (B) a second PEgRNA or one or more polynucleotides encoding the second PEgRNA, (C) a prime editor comprising a Cas9 nickase having a nuclease inactivation mutation in a HNH domain and a reverse transcriptase, or one or more polynucleotides encoding the prime editor, and (D) a Pa01 recombinase or one or more polynucleotides encoding the Pa01 recombinase, wherein the first PEgRNA comprises: (i) a first spacer that is complementary to a first search target sequence on a first strand of a target gene, (ii) a first gRNA core capable of binding to the Cas9 nickase; and (iii) a first extension arm comprising a first editing template and a first primer binding site (PBS), wherein the second PEgRNA comprises: (i) a second spacer that is complementary to a second search target sequence on a second strand of the target gene complementary to the first strand, (ii) a second gRNA core capable of binding to the Cas9 nickase; and (iii) a second extension arm comprising a second editing template and a second PBS, wherein the first editing template comprises a region of complementarity to the second editing template, wherein the first editing template and the second editing template each encodes all or a fragment of a recombinase recognition sequence (RSS) or the reverse complement thereof, wherein the first editing template encodes at least a 5′ portion of the RSS or the reverse complement thereof, wherein the second editing template encodes at least a 3′ portion of the RSS or the reverse complement thereof, wherein at least 10 nucleotides at the 5′ end of the first and the second editing templates have perfect reverse complementarity to each other, and wherein the RSS is recognized by the Pa01 recombinase.
184. Embodiment 184. The prime editing system of embodiment 183, wherein at least 15, 20, 25, or 30 nucleotides at the 5′ end of the first and second editing templates have perfect reverse complementarity to each other.
185. Embodiment 185. The prime editing system of any one of embodiments 183-184, wherein the first editing template encodes the RSS.
186. Embodiment 186. The prime editing system of any one of embodiments 183-185, wherein the second editing template encodes the RSS.
187. Embodiment 187. The prime editing system of any one of embodiments 183-186, wherein the first editing template comprises a 5′ fragment of an RTT listed in RTT pair 2 or 3 of Table 6 and wherein the second editing template comprises a full length or 5′ fragment of the corresponding RTT pair and wherein at least 10 nucleotides at the 5′ end of the first and second editing templates have perfect reverse complementarity to each other.
188. Embodiment 188. The prime editing system of any one of embodiments 183-187, wherein the second editing template comprises a 5′ fragment of an RTT listed in RTT pair 2 or 3 of Table 6 and wherein the first editing template comprises a full length or 5′ fragment of the corresponding RTT pair and wherein at least 10 nucleotides at the 5′ end of the first and second editing templates have perfect reverse complementarity to each other.
189. Embodiment 189. The prime editing system of any one of embodiments 183-188, wherein the first PBS comprises a region of complementarity to a region upstream of a nick site in the second strand, and wherein the second PBS comprises a region of complementarity to a region upstream of a nick site in the first strand.
190. Embodiment 190. The prime editing system of any one of embodiments 183-189, wherein the prime editing system further comprises a DNA polynucleotide that comprises (i) a second RSS recognized by the Pa01 recombinase, and (ii) a donor sequence.
191. Embodiment 191. The prime editing system of any one of embodiments 183-190, wherein the target gene is TRAC.
192. Embodiment 192. A method for integrating a donor sequence in a target gene, the method comprising contacting the target gene with the prime editing system of any one of the embodiments 183-191.

1. A prime editing system comprising: (A) a first prime editing guide RNA (PEgRNA) or one or more polynucleotides encoding the first PEgRNA and (B) a second PEgRNA or one or more polynucleotides encoding the second PEgRNA,
wherein the first PEgRNA comprises from 5′ to 3′ in a contiguous sequence in a single molecule:
(i) a first spacer that is complementary to a first search target sequence on a first strand of a TRAC gene,
(ii) a first gRNA core capable of binding to a Cas9 protein; and
(iii) a first extension arm comprising:
(a) a first editing template encoding a recombinase recognition site (RRS) or its reverse complement, and
(b) a first primer binding site (PBS),
wherein the first spacer is SEQ ID NO: 61 and the first PBS is SEQ ID NO: 71, or wherein the first spacer is SEQ ID NO: 4 and the first PBS is SEQ ID NO: 15; and
wherein the second PEgRNA comprises, from 5′ to 3′ in a contiguous sequence in a single molecule:
(i) a second spacer that is complementary to a second search target sequence on a second strand of the TRAC gene complementary to the first strand,
(ii) a second gRNA core capable of binding to a Cas9 protein; and
(iii) a second extension arm comprising:
(a) a second editing template that is completely complementary to the first editing template, and
(b) a second PBS,
wherein the second spacer is SEQ ID NO: 177, and wherein the second PBS is SEQ ID NO: 186, or wherein the second spacer is SEQ ID NO: 522 and the second PBS is SEQ ID NO: 531.

2. The prime editing system of claim 1, wherein first spacer is SEQ ID NO: 61 and the first PBS is SEQ ID NO: 71, and wherein the second spacer is SEQ ID NO: 177 and the second PBS is SEQ ID NO: 186.

3. The prime editing system of claim 1, wherein the first spacer is SEQ ID NO: 4 and the first PBS is SEQ ID NO: 15, and wherein the second spacer is SEQ ID NO: 522 and the second PBS is SEQ ID NO: 531.

4.-14. (canceled)

15. The prime editing system of claim 1, wherein the first gRNA core, the second gRNA core, or both comprise SEQ ID NO: 590.

16.-22. (canceled)

23. The prime editing system of claim 1, wherein the first editing template encodes the RRS.

24. (canceled)

25. The prime editing system of claim 1, wherein the RRS is an attB sequence recognized by a Bxb1 recombinase.

26. The prime editing system of claim 1, wherein the RRS is an attP sequence recognized by a Bxb1 recombinase.

27.-58. (canceled)

59. The prime editing system of claim 1, wherein the first PEgRNA and/or the second PEgRNA further comprises a 3′ motif, wherein the 3′ motif is UUUU.

60. The prime editing system of claim 1, wherein the first PEgRNA and/or the second PEgRNA further comprises 5′mN*mN*mN* and 3′ mN*mN*mN*N modifications, where m indicates that the nucleotide contains a 2′-O-Me modification and a * indicates the presence of a phosphorothioate bond.

61. The prime editing system of claim 1, further comprising a prime editor or one or more polynucleotides encoding the prime editor, wherein the prime editor comprises (a) a Cas9 nickase having a nuclease inactivating mutation in a HNH domain and (b) a reverse transcriptase.

62.-63. (canceled)

64. The prime editing system of claim 61, wherein the prime editor is a fusion protein.

65.-66. (canceled)

67. The prime editing system of claim 61, further comprising a recombinase that recognizes the RRS or one or more polynucleotides encoding the recombinase.

68.-70. (canceled)

71. The prime editing system of claim 67, further comprising a DNA polynucleotide that comprises (a) a donor sequence and (b) a second RRS recognized by the recombinase.

72. The prime editing system of claim 71, wherein (i) the RRS comprises a Bxb1 attB sequence provided in Table 5, and the second RRS comprises a corresponding attP sequence provided in Table 5.

73. (canceled)

74. The prime editing system of claim 72, wherein the donor sequence comprises an open reading frame that encodes a polypeptide.

75. The prime editing system of claim 74, wherein the donor sequence encodes a chimeric antigen receptor (CAR).

76.-86. (canceled)

87. An in vitro or ex vivo method of making a modified cell, the method comprising contacting a cell with the prime editing system of claim 61.

88. (canceled)

89. The method of claim 87, wherein the cell is a mammalian cell.

90. The method of claim 87, wherein the cell is a human cell.

91. The method of claim 87, wherein the cell is an immune cell, optionally wherein the cell is a T cell.

92.-107. (canceled)

108. The prime editing system of claim 71, wherein the RRS comprises a Bxb1 attP sequence provided in Table 5, and the second RRS comprises a corresponding attB sequence provided in Table 5.
